(19) United States
(12) Patent Application Publication
(10) Pub. No.: US 2009/0253583 A1
(43) Pub. Date: Oct. 8, 2009

(54) Hematological Cancer Profiling System

(75) Inventor: Thillainathan Yoganathan, Richmond (CA)

Correspondence Address: MORRISON & FOERSTER LLP, 12531 HIGH BLUFF DRIVE, SUITE 100, SAN DIEGO, CA, 92130-2040, US

(73) Assignee: MED BIOGENE INC., Vancouver, BC (CA)

(21) Appl. No.: 11/576,143

(22) PCT Filed: Sep. 27, 2005
(86) PCT No.: PCT/CA05/01464

§ 371 Date: Jul. 21, 2008

Related U.S. Application Data
(60) Provisional application No. 60/613,980, filed on Sep. 27, 2004.

(51) Int. Cl. C40B 40/06; C40B 30/04; C40B 50/00; C40B 30/00; C07H 21/00

(52) U.S. Cl. 506/9; 506/16; 506/23; 506/7; 536/24.31

(57) ABSTRACT

A system for profiling lymphoma is provided that is based on the identification of sets of genes, which are characterized in that changes in expression of each gene within a set of genes can be correlated to one or more features of a specific lymphoma. The lymphoma profiling system provides for sets of “lymphoma profiling” genes and further provides for combinations of polynucleotide probes derived from one or more of the lymphoma profiling sets. These combinations of polynucleotide probes can be provided in solution or as an array. The combination of probes and the arrays can be used for disease prognosis, diagnosis, staging or grading, treatment management, monitoring of disease progression, predicting disease outcome or complications, and the like.

### FIELD OF THE INVENTION

The present invention relates to the field of cancer diagnosis and profiling and, in particular, to tools for diagnosing and profiling hematological cancers.

### BACKGROUND OF THE INVENTION

Hematological cancers are cancers of the blood and lymphatic system. These cancers usually affect the white blood cells (disease and infection-fighting cells) rather than the red blood cells (oxygen-carrying cells), and can occur in the marrow where all blood cells are made, or in the lymph nodes and other lymph tissues that the white blood cells flow through. Common hematological cancers are leukemia, lymphoma, and myeloma.

Lymphoma is a type of cancer affecting cells in the lymph system, and is most commonly caused by mutations in the genetic material of a B-cell or T-cell lymphocyte. Lymphocytes with these mutations lose their ability to control their own multiplication and are, therefore, able to overtake healthy tissue and form tumors. The type of mutation and the stage of development at which it occurs determine what class or type of lymphoma will arise. Since lymphocytes undergo several stages of hematopoietic differentiation during development from stem cell to mature B- or T-cell, many classes of lymphoma have been identified (Staudt L M. N Engl J Med. 2003; 348(18):1777-85. [Erratum: N Engl J Med. 2003; 348(25):2588.]) Correct identification of lymphoma types and/or sub-types is critical since different types of lymphomas have varied prognoses, and treatments for the various types may be entirely different (Soukup J, Krskova L, Mrhalova M, et al. Cas Lek Cesk. 2003; 142(7):417-2). Furthermore, recent studies have shown that within a single disease classification, the response to treatment between individuals can vary. For example, a group of patients were diagnosed with diffuse large cell B-cell lymphoma (DLBCL) by pathologists and treated. Some of these patients responded well to the treatment and were completely cured, but others did not and succumbed to the disease within a year (Alizadeh A A, Eisen M B, Davis R E et al. Nature. 2000; 403(6769):503-11).

In broadest terms, lymphomas can be classified as Hodgkin's disease or lymphoma (HD or HL) and non-Hodgkin's lymphoma (NHL). NHL can be further classified according to the type of lymphocyte affected, i.e. B-cell lymphomas or T-cell lymphomas.

The World Health Organization (WHO) has grouped non-Hodgkin's lymphomas into classes based on a combination of morphology, immunology, genetic features, and clinical features. According to this classification system, mature (peripheral) B-cell lymphomas have been classified into the following types: B-cell chronic lymphocytic leukemia/small lymphocytic lymphoma (CLL/SLL), B-cell prolymphocytic leukemia, lymphoplamacytic lymphoma, splenic marginal zone B-cell lymphoma, nodal marginal zone B-cell lymphoma, hairy cell leukemia, plasma cell myeloma/plasmacytoma, follicular lymphoma (FL), mantle cell lymphoma (MCL), Burkitt's lymphoma, and DLBCL. Hodgkin's lymphoma is also of B-cell origin.

DLBCL is an aggressive form of lymphoma that has a mortality rate of 50-60%. The WHO has sub-classified DLBCL into broad categories, thus making an accurate diagnosis difficult (Alizadeh A A, Eisen M B, Davis R E et al. Nature. 2000; 403(6769):503-11). More recently, however, molecular profiling of hundreds of DLBCL patient samples has resulted in the identification of the following sub-types of DLBCL based on gene expression patterns: a sub-type derived from differentiated activated peripheral blood B-cells (ABC), a second sub-type derived from the undifferentiated germinal centers of lymph nodes (GC), a third sub-type called mediastinal large B-cell lymphoma (MLBCL), and a fourth sub-type that remains largely heterogeneous (Rosenwald A, Wright G, Chan W C et al. N Engl J Med. 2002, 346(25):1937-47; Rosenwald A, Wright G, Leroy K. et al. J Exp Med. 2003, 198(6):851-62; Wright G, Tan B, Rosenwald A, et al. Proc Natl Acad Sci USA. 2003, 100(17):9991-6; Savage K J, Monti S, Kutok J L, et al. Blood. 2003, 102(12):3871-9).

T-cell lymphomas have been classified into the following types: lymphoblastic lymphoma, anaplastic large cell lymphoma (ALCL), subcutaneous T-cell lymphoma, mycosis fungoids/Sézary's syndrome, peripheral T-cell lymphomas, angioimmunoblastic lymphoma, angiocentric lymphoma (nasal T-cell lymphoma), intestinal T-cell lymphoma, and adult T-cell lymphoma/leukemia.

Despite efforts of the WHO and other organizations to classify lymphomas, these cancers are difficult to classify since there is no single marker that clinicians can consider to classify all of the various types of lymphoma (Harris N L, Jaffe E S, Diebold J et al. Ann Oncol. 2000; 11 Suppl 1:3-10). In most cases, physicians must employ a variety of techniques to clearly identify a patient's disease. These techniques include gross and microscopic morphological examination, detection of characteristic chromosomal rearrangements, and detection of aberrant gene expression. The complexity and subjectivity involved in interpreting the results obtained using these techniques add further challenges to clinicians and pathologists trying to diagnose and treat a patient with lymphoma.

Leukemia is a cancer of the white blood cells that starts in the bone marrow and spreads to the blood, lymph nodes, and other organs. Both children and adults can develop leukemia, which is a complex disease with many different types and sub-types. The treatment given and the outlook for patients with leukemia varies greatly according to the exact type and other individual factors. Leukemias are classified into types based on the kind of blood cell they involve, either lymphoid or myeloid, as well as the speed of disease progression, either acute or chronic. Acute lymphocytic leukemia (ALL) is the most common form of leukemia among children, often striking during infancy. Acute myelogenous leukemia (AML) occurs in both adults and children, while cases of chronic lymphocytic leukemia (CLL) and chronic myelogenous leukemia (CML) are seen mainly in adults. Many leukemia subtypes exist within the various types. Subtypes are often defined by a specific mutation they share. Common subtypes include; MLL, T-ALL (T-cell acute lymphoblastic leukemia), BCR-ABL, TEL-AML1, E2A-PBX1, ALL with t(4;11). As for lymphoma, accurately distinguishing between the different types and subtypes of leukemia is critical for making correct diagnoses and for choosing the most beneficial treatment protocol. Current methods used to diagnose leukemia and distinguish types and subtypes of leukemia involve examination of a patient's medical history, a physical examination, complete blood counts, a bone marrow examination, cytogenetic studies, molecular diagnostics and immunophenotyping, followed by, in some cases assigning the patient to a specific risk group. This process can be difficult and expensive.

The advent of DNA array technology has allowed high-throughput analysis of gene expression in both lymphoma and leukemia. For example, “molecular profiling” of lymphomas has facilitated the identification of predictive factors or biomarkers that may potentially be used to characterize lymphomas. The “Lymphochip,” for example, has been used to identify sub-types of one specific type of lymphoma, DLBCL, and contains a total of 17,856 cDNA clones, the majority of which are derived from a germinal centre B-cell library, as well as cDNA clones derived from DLBCL, FL, MCL, and CLL libraries (Alizadeh A A, Eisen M B, Davis R E et al. Nature. 2000; 403(6769):503-11)). An oligonucleotide array has also been described, which was used to analyze the expression of 6,817 genes in diagnostic tumor specimens from DLBCL patients (Shipp M A, Ross K N, Tamayo P et al. Nat Med. 2002 January; 8(1):68-74). This array was used to predict outcome (cured vs. fatal) in this specific type of lymphoma and to identify potential therapeutic targets.

Other publications have described the use of oligonucleotide microarrays to accurately distinguish subtypes of leukemia. A commercially available microarray containing probes for 6817 genes was used to classify leukemia and identify a set of genes as class predictors (Golub T R, Slonim D K, Tamayo P, Huard C, Gaasenbeek M, Mesirov J P, Coller H, Loh M L, Downing J R, Caligiuri M A, Bloomfield C D and E S Lander. Science 1999 Oct. 15; 286(5439): 531-537). Others have used a commercially available microarray (Affymetrix U133A) to distinguish various subtypes of pediatric ALL with 96% accuracy (Ross M E, Zhou X, Song G, Shurtleff S A, Girtman K, Williams W K, Liu H C, Mahfouz R, Raimondi S C, Lenny N, Patel A and J R Downing. Blood 2003 Oct. 15; 102(8): 2951-2959).

In addition, U.S. Patent Application No. 20020110820 describes fourteen collections of 1000 genes, each representing a different cancer, including lymphoma and leukemia. Methods of using these collections to identify a tumor, predict the likelihood of tumor development, diagnose a tumor, or identify a compound for use in treating cancer are also described. The patent application further describes an oligonucleotide array containing a plurality of oligonucleotide probes specific for the genes in these collections.

U.S. Patent Application No. 20030175761 describes a group of 120 genes whose expression patterns allow differentiation between benign lymph node tissue, FL, MCL, and SLL. This patent application further describes nucleic acid arrays containing probes for these genes. U.S. Patent Application No. 20030219760 describes methods for diagnosing biological states or conditions based on ratios of gene expression data from tissue samples, such as cancer tissue samples. The application describes a method based on focused microarray-based profiling that permits confirmation of the presence of malignant pleural mesothelioma. The application also indicates that the method is applicable to a variety of other cancers, including lymphomas and leukemias, and lists sets of genes that were selected based on analysis of gene expression data presented in the prior art. The listed genes include genes that are differentially expressed in different sub-types of DLBCL, that are over-expressed in DLBCL and FL, and that are over-expressed in DLBCL of good and poor outcome.

U.S. Patent Application No. 20040018513 describes methods and compositions useful for diagnosing and choosing treatment for leukemia patients. These methods are based on analysis of gene expression using HG_U95Av2 Affymetrix™ oligonucleotide arrays, and relate to patients that can be assigned to a leukemia risk group selected from T-ALL, E2A-PBX1, TEL-AML1, BCR-ABL, MLL, Hyperdiploid >50, and a risk group called “Novel,” which is distinguishable from the others in the list based on expression profiling. The methods can be used to assign a subject affected by leukemia to a leukemia risk group, predict increased risk of relapse, predict increased risk of developing secondary acute myeloid leukemia, determine prognosis, choose therapy, and monitor disease state. The application also describes arrays having capture probes for the differentially-expressed genes described therein.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a hematological cancer profiling system. In accordance with one aspect of the present invention, there is provided a system for profiling a hematological cancer comprising at least ten polynucleotide probes, each of said probes being between about 15 and about 500 nucleotides in length and comprising a sequence corresponding to, or complementary to, an mRNA transcribed from a gene selected from the group of genes set forth in Table 1, wherein the level of expression of said gene is indicative of one or more features of said hematological cancer.

In accordance with another aspect of the present invention, there is provided a use of the system according to the present invention for the preparation of a nucleic acid array.

In accordance with another aspect of the present invention, there is provided a method of profiling a hematological cancer in a subject comprising: (a) providing one or more gene sets, each gene set comprising at least five genes selected from the genes set forth in Table 1, wherein the expression level of each gene in said one or more gene sets is indicative of a feature of a heinatological cancer; (b) determining the expression level of each gene in said one or more gene sets in a test sample obtained from said subject to provide an expression pattern profile, and (c) comparing said expression pattern profile with a reference expression pattern profile.

In accordance with another aspect of the present invention, there is provided a nucleic acid array comprising at least ten polynucleotide probes immobilized on a solid support, each of said probes being between about 15 and about 500 nucleotides in length and comprising a sequence corresponding to, or complementary to, an mRNA transcribed from a gene selected from the group of genes set forth in Table 1, wherein the level of expression of said gene is indicative of one or more features of said hematological cancer.

In accordance with another aspect of the present invention, there is provided a polynucleotide probe between about 15 and about 500 nucleotides in length and comprising a sequence corresponding to, or complementary to, an mRNA transcribed from a gene selected from the group of genes set forth in Table 1, wherein said probe comprises at least 15 consecutive nucleotides of a sequence as set forth in any one of SEQ ID NOs: 1-4530.

In accordance with another aspect of the present invention, there is provided a set of genes having an expression pattern representative of one or more features of a hematological cancer and comprising at least ten genes selected from: (a) at least ten genes selected from the genes set forth in Table 32; (b) at least ten genes selected from the genes set forth in Table 33; (c) at least ten genes selected from the genes set forth in Table 34; (d) at least ten genes selected from the genes set forth in Table 35; (e) at least ten genes selected from the genes set forth in Table 36; (f) at least ten genes selected from the genes set forth in Table 37; (g) at least ten genes selected from the genes set forth in Table 38; (h) at least ten genes selected from the genes set forth in Table 39; (i) at least ten genes selected from the genes set forth in Table 40, and (j) at least ten genes selected from the genes set forth in Table 41.

In accordance with another aspect of the present invention, there is provided a library of genes for profiling a hematological cancer, comprising the genes as set forth in Table 1.

In accordance with another aspect of the present invention, there is provided a computer-readable medium comprising one or more digitally-encoded expression pattern profiles representative of a set of genes according to any one of claims 38-41, each of said one or more expression pattern profiles being associated with one or more values wherein each of said one or more values is correlated with one of said one or more features of a hematological cancer.

### BRIEF DESCRIPTION OF FIGURES

FIG. 1 depicts a hierarchical clustering image of DLBCL signature genes in DLBCL samples versus control.

FIG. 2 depicts a hierarchical clustering image of FL signature genes in FL samples versus control.

FIG. 3 depicts a hierarchical clustering image of HL signature genes in HL samples versus control.

FIG. 4 depicts a hierarchical clustering image of MCL signature genes in MCL samples versus control.

FIG. 5 depicts a hierarchical clustering image of MZL signature genes in MZL samples versus control.

FIG. 6 depicts a hierarchical clustering image of SLL signature genes in SLL samples versus control.

FIG. 7 depicts a hierarchical clustering image of TCL signature genes in TCL samples versus control.

FIG. 8 depicts a hierarchical clustering image of lymphoma signature genes in 23 lymphoma samples versus control.

FIG. 9 depicts a hierarchical clustering image of leukemia signature genes in 4 leukemia samples versus control, and in 3 lymphoma samples.

FIG. 10 depicts a hierarchical clustering image of CLL signature genes in CLL samples versus control.

FIG. 11 depicts a hierarchical clustering image of AML signature genes in AML samples versus control.

FIG. 12 depicts a hierarchical clustering image of T-ALL signature genes in T-All samples versus control.

### DETAILED DESCRIPTION OF THE INVENTION

The present invention provides for a system for profiling hematological cancers. This system is based on the identification of a pool, or library, of genes that are characterized in that changes in expression of each of the genes can be correlated to one or more features of a hematological cancer. The library provided by the present invention can be used as a resource from which sets of “hematological cancer profiling” genes can be selected, each set representing a specific hematological cancer, for example, lymphoma or leukemia, or a type or sub-type of lymphoma or leukemia. The level of expression of each gene in a hematological cancer profiling set is indicative of one or more features of the hematological cancer represented by that set of genes. A combination of polynucleotide probes (a “hematological cancer profiling combination”) that comprises probes derived from the sequences of the genes of one or more hematological cancer profiling sets can then be prepared in order to profile one or more hematological cancers of interest. The system of the present invention thus provides the user with the flexibility of assessing the type(s) and/or feature(s) of the hematological cancer(s) that are of specific interest by selecting an appropriate hematological cancer profiling combination. In accordance with the present invention, the hematological cancer is selected from the group of: lymphoma and leukemia. Non-limiting examples of features of these cancers that can be assessed with system of the present invention include presence/absence, type, subtype, stage, progression, grade, aggressivity, outcome, survival and drug-responsiveness of hematological cancers, and the like.

The system of the present invention thus provides for sets of “hematological cancer profiling” genes selected from the library of genes. The system further provides for combinations of polynucleotide probes (“hematological cancer profiling combinations”) derived from the sequences of the genes of one or more hematological cancer profiling sets. A hematological cancer profiling combination thus comprises a plurality of probes that represent one or more hematological cancer profiling sets.

As indicated above, the system of the present invention allows for hematological cancer profiling combinations to be selected that are tailored to assess type(s) and/or feature(s) of the hematological cancer(s) of interest. The combination of probes thus may be tailored as desired such that it represents a single feature of a hematological cancer, multiple features of a hematological cancer, a single feature of multiple hematological cancers or multiple features of multiple hematological cancers.

The system provides for combinations of probes in solution, for example, for use in standard solution hybridization techniques or for use in quantitative PCR applications, as well as combinations of probes in an immobilised format, for example, as an array. The system can be used to analyse the expression pattern of genes belonging to one or more hematological cancer profiling (HCP) sets in a blood or biopsy sample from a patient having, suspected of having, or suspected of being at risk of developing, a hematological cancer. The resulting information allows the determination of one or more features of the hematological cancer such as those described above, and is, therefore, useful in disease prognosis, diagnosis, staging or grading, treatment management, monitoring of disease progression, predicting disease outcome or complications, and the like. The system can thus be used to profile a hematological cancer selected from the group of lymphoma and leukemia.

### DEFINITIONS

Unless defined otherwise, all technical and scientific terms used herein have the same meaning as commonly understood by one of ordinary skill in the art to which this invention belongs.

As used herein, the term “about” refers to a +/−10% variation from the nominal value. It is to be understood that such a variation is always included in any given value provided herein, whether or not it is specifically referred to.

The term “feature of a hematological cancer,” as used herein, refers to a characteristic of a hematological cancer. Such characteristics include fundamental aspects such as presence/absence of the disease in a subject and type of hematological cancer that are useful in diagnosis, as well as characteristics such as subtype, stage, progression, grade, aggressivity, drug-responsiveness, and the like, which are useful for disease management and patient care.

The term “gene,” as used herein, refers to a segment of nucleic acid that encodes an individual protein or RNA (also referred to as a “coding sequence” or “coding region”) together with associated regulatory regions such as promoters, operators, terminators and the like, that may be located upstream or downstream of the coding sequence.

The term “target gene,” as used herein, refers to a gene, the expression of which is to be detected using a polynucleotide probe of a hematological cancer profiling combination. In the context of the present invention, the target gene is a member of a hematological cancer profiling set.

The term “target mRNA,” as used herein, refers to an mRNA transcribed from a target gene.

The terms “oligonucleotide” and “polynucleotide” as used interchangeably in the present application refer to a polymer of greater than one nucleotide in length of ribonucleic acid (RNA), deoxyribonucleic acid (DNA), hybrid RNA/DNA, modified RNA or DNA, or RNA or DNA mimetics. The polynucleotides may be single- or double-stranded. The terms include polynucleotides composed of naturally-occurring nucleobases, sugars and covalent internucleoside (backbone) linkages as well as polynucleotides having non-naturally-occurring portions which function similarly. Such modified or substituted polynucleotides are well-known in the art and for the purposes of the present invention, are referred to as “analogues.”

The terms “probe” and “polynucleotide probe,” as used herein, refer to a polynucleotide that is capable of hybridizing to a target gene or target mRNA and includes polynucleotides in solution as well as those that are immobilized to a solid substrate, e.g. in an array.

By “gene expression pattern” or “expression pattern” is meant the level of gene expression of one or more target genes in a test sample, for example, genes of a hematological cancer profiling set as assessed by methods described herein. The “level of gene expression” refers to an absolute or relative amount of the transcription product of the target gene(s). Typically, the level of expression is measured relative to a reference sample and can be increased (up-regulated), decreased (down-regulated) or unchanged relative to the reference sample. The gene expression pattern can be measured at a single time point or over a period of time.

By “altered gene expression” is meant an increase or decrease in gene expression, as described below.

By “decrease in gene expression” is meant a lowering of the level of expression of a gene relative to a reference sample. Typically, the decrease is at least 10% relative to the reference. In one embodiment, a decrease in gene expression refers to a decrease in expression of the gene by at least 25%. In other embodiments, a decrease in gene expression refers to a decrease in expression of the gene by at least 30%, 40%, 50%, 60%, 70%, 80%, and 90%. Alternatively, a decrease in gene expression is at least 2-fold relative to the reference. In further embodiments, a decrease in gene expression refers to a decrease in expression of the gene by at least 3, 5, 7, or 10-fold relative to the reference.

By “increase in gene expression” is meant a raising of the level of expression of a gene relative to a reference sample. Typically, the increase is at least 10% relative to the reference. In one embodiment, an increase in gene expression refers to a decrease in expression of the gene by at least 25%. In other embodiments, an increase in gene expression refers to an increase in expression of the gene by at least 30%, 40%, 50%, 60%, 70%, 80%, and 90%. Alternatively, an increase in gene expression is at least 2-fold relative to the reference. In further embodiments, an increase in gene expression refers to an increase in expression of the gene by at least 3, 5, 7, or 10-fold relative to the reference.

The term “selectively hybridize,” as used herein, refers to the ability of a polynucleotide probe bind detectably and specifically to a target gene or nucleic acids derived therefrom. A polynucleotide probe selectively hybridizes to a target gene or nucleic acids under hybridization and wash conditions that minimize appreciable amounts of detectable binding to non-specific nucleic acids. High stringency conditions can be used to achieve selective hybridization conditions as known in the art and discussed herein. Typically, hybridization and washing conditions are performed at high stringency according to conventional hybridization procedures. Washing conditions are typically 1-3×SSC, 0.1-1% SDS, 50-70° C. with a change of wash solution after about 5-30 minutes.

The terms “corresponding to” or “corresponds to” indicates that a polynucleotide sequence is identical to all or a portion of a reference polynucleotide sequence. In contradistinction, the term “complementary to” is used herein to indicate that the polynucleotide sequence is identical to all or a portion of the complementary strand of a reference polynucleotide sequence. For illustration, the nucleotide sequence “TATAC” corresponds to a reference sequence “TATAC” and is complementary to a reference sequence “GTATA.”

The following terms are used herein to describe the sequence relationships between two or more polynucleotides: “reference sequence,” “window of comparison,” “sequence identity,” and “percent sequence identity.” A “reference sequence” is a defined sequence used as a basis for a sequence comparison; a reference sequence may be a subset of a larger sequence, for example, as a segment of a full-length cDNA, or gene sequence, or may comprise a complete cDNA, or gene sequence. Generally, a reference polynucleotide sequence is at least 20 nucleotides in length, and often at least 50 nucleotides in length.

A “window of comparison”, as used herein, refers to a conceptual segment of the reference sequence of at least 15 contiguous nucleotide positions over which a candidate sequence may be compared to the reference sequence and wherein the portion of the candidate sequence in the window of comparison may comprise additions or deletions (i.e. gaps) of 20 percent or less as compared to the reference sequence (which does not comprise additions or deletions) for optimal alignment of the two sequences. The present invention contemplates various lengths for the window of comparison, up to and including the full length of either the reference or candidate sequence. Optimal alignment of sequences for aligning a comparison window may be conducted using the local homology algorithm of Smith and Waterman (Adv. Appl. Math. (1981) 2:482), the homology alignment algorithm of Needleman and Wunsch (J. Mol. Biol. (1970) 48:443), the search for similarity method of Pearson and Lipman (Proc. Natl. Acad. Sci. (U.S.A.) (1988) 85:2444), using computerized implementations of these algorithms (such as GAP, BESTFIT, FASTA, and TFASTA in the Wisconsin Genetics Software Package Release 7.0, Genetics Computer Group, 573 Science Dr., Madison, Wis.), using publicly available computer software such as ALIGN or Megalign (DNASTAR), or by inspection. The best alignment (i.e. resulting in the highest percentage of identity over the comparison window) is then selected.

The term “sequence identity” means that two polynucleotide sequences are identical (i.e. on a nucleotide-by-nucleotide basis) over the window of comparison.

The term “percent (%) sequence identity,” as used herein with respect to a reference sequence is defined as the percentage of nucleotides in a candidate sequence that are identical with the nucleotides in the reference polynucleotide sequence over the window of comparison after optimal alignment of the sequences and introducing gaps, if necessary, to achieve the maximum percent sequence identity.

### 1. Hematological Cancer Profiling System

The system of the present invention is based on the identification of genes whose expression level is altered in a subject having a hematological cancer when compared to a reference subject and thus are indicative of a feature of the hematological cancer. For example, if the hematological cancer is lymphoma or leukemia, then depending on the feature in question, the reference subject can be, for example, a disease-free subject, a subject having a different type or subtype of lymphoma or leukemia, a subject undergoing a different therapeutic regimen, a subject with a different stage or grade of lymphoma or leukemia, a subject with an indolent form of disease, etc. Such genes are candidates for inclusion in one or more of the hematological cancer profiling (HCP) sets of the invention. Once genes have been selected for the HCP set(s), probes can be designed that specifically hybridise to the genes within the set. Combinations of the probes can then be formed that comprise probes representing those HCP sets that correlate with the hematological cancer(s) and/or feature(s) of the hematological cancer(s) that are of interest.

### 1.1 Hematological Cancer Profiling (HCP) Sets

An HCP set comprises one or more genes related to a hematological cancer, such as lymphoma or leukemia, i.e. a gene whose expression pattern is indicative of a selected lymphoma or leukemia, or a feature of a selected lymphoma or leukemia. For example, the level of expression of the gene can be indicative of the presence of a particular lymphoma or subtype thereof; the stage, grade, or aggressivity of a lymphoma or subtype thereof; the progression of a lymphoma or subtype thereof (for example, whether the lymphoma is localized, regional, or metastatic); the drug-responsiveness of a lymphoma or subtype thereof (for example, whether the lymphoma is drug-sensitive, drug-resistant or multi-drug resistant); the likelihood of transformation of one type of lymphoma to another (for example, transformation of FL into DLBCL); whether the lymphoma is refractory (i.e. not responsive to treatment); likelihood for the lymphoma to be fatal; mutational status of a lymphoma, and the like. Alternatively, the level of expression of the gene in an HCP set may reflect whether a subject affected by leukemia has a particular sub-type of leukemia, an increased risk of relapse, has an increased risk of developing secondary acute myeloid leukemia, prognosis for the subject with leukemia, selection of appropriate therapy for leukemia, the drug-responsiveness of leukemia or sub-type thereof, or the progression of the leukemia.

As is known in the art, some genes have been identified that correlate with more than one hematological cancer. For example, the expression level of a gene may be indicative of a feature of both lymphoma and leukemia. Moreover, some genes have been identified that correlate with more than one feature of a specific hematological cancer. For example, the expression level of a gene may be correlated to transformation of one type of lymphoma to another as well as being indicative of the subtype of that lymphoma. Such genes are also suitable for inclusion in the HCP sets of the present invention.

Each gene selected for inclusion in a particular HCP set relates to the same hematological cancer or type, or sub-type of a hematological cancer. As indicated above, hematological cancers contemplated by the present invention are selected from lymphoma or leukemia. Thus, for example, all of the genes of an HCP set may relate to lymphoma in general. Alternatively, exemplary types of lymphomas for which an HCP set can be formed include, but are not limited to, small lymphocytic lymphoma (SLL), B-cell prolymphocytic leukemia, lymphoplamacytic lymphoma, splenic marginal zone B-cell lymphoma, nodal marginal zone B-cell lymphoma, hairy cell leukemia, plasma cell myeloma/plasmacytoma, follicular lymphoma (FL), mantle cell lymphoma (MCL), Burkitt's lymphoma, diffuse large cell B-cell lymphoma (DLBCL), Hodgkin's lymphoma, lymphoblastic lymphoma, anaplastic large cell lymphoma (ALCL), cutaneous T-cell lymphoma, mycosis fungoids/Sézary's syndrome, peripheral T-cell lymphomas, angioimmunoblastic lymphoma, angiocentric lymphoma (nasal T-cell lymphoma), intestinal T-cell lymphoma, and adult T-cell lymphoma/leukemia. In one embodiment of the invention, genes selected for inclusion in an HCP set relate to a type of lymphoma selected from the group of: DLBCL, FL, CLL/SLL, MCL, HL, and ALCL.

Likewise, the present invention further contemplates that all of the genes of an HCP set may relate to leukemia in general. Alternatively, all of the genes of an HCP set may relate to a sub-type of leukemia. Exemplary sub-types of leukemias for which an HCP set can be designed include, but are not limited to B-cell chronic lymphocytic leukemia (CLL), chronic myelogenous leukemia (CML), acute lymphocytic leukemia (ALL), acute myelogenous leukemia (AML), T-ALL, MLL, BCR-ABL, TEL-AML1, E2A-PBX1, ALL with t(4;11). In one embodiment of the invention, genes selected for inclusion in an HCP set relate to a leukemia selected from the group of CLL, AML, and T-ALL.

With respect to CLL, a worker skilled in the art would understand that this sub-type of hematological cancer may be considered as a sub-type of lymphoma. Alternatively, it may be considered as a sub-type of leukemia.

Appropriate candidate genes for inclusion in the HCP sets can be selected from genes known in the art to be markers for a particular feature of a hematological cancer. Such genes can be identified from publicly available databases using a variety of “data mining” approaches known in the art. Alternatively, candidate genes can be identified by screening a nucleic acid library derived from a hematological cancer exhibiting a specific feature and selecting for genes whose expression level is modulated in this library when compared to a reference nucleic acid library. Methods of creating nucleic acid libraries are well known in the art (see, for example, Ausubel et al., (1997 & updates) Current Protocols in Molecular Biology, Wiley & Sons, New York).

In one embodiment of the present invention, candidate genes for inclusion in the HCP sets are identified by data mining. As is known in the art, publication and sequence databases can be mined using a variety of search strategies. For example, currently available scientific and medical publication databases such as Medline, Current Contents, OMIM (online Mendelian inheritance in man), various Biological and Chemical Abstracts, Journal indexes, and the like can be searched using term or key-word searches, or by author, title, or other relevant search parameters. Many such databases are publicly available, and strategies and procedures for identifying publications and their contents, for example, genes, other nucleotide sequences, descriptions, indications, expression pattern, etc, are well known to those skilled in the art. Numerous databases are available through the internet for free or by subscription, see, for example, the National Center Biotechnology Information (NCBI), Infotrieve, Thomson ISI, and Science Magazine (published by the AAAS) websites. Additional or alternative publication or citation databases are also available that provide identical or similar types of information, any of which can be employed in the context of the invention. These databases can be searched for publications describing altered gene expression between features of hematological cancers such as types of hematological cancers, for example types of lymphoma or leukemia, or subtypes of a specific hematological cancer, such as subtypes of lymphoma or leukemia.

For example, genes can initially be selected by consulting publications to identify genes that have been shown to be indicative of features of hematological cancers. The methods used to determine the expression level of these genes can include a variety of methods including PCR, Northern blots and microarray studies. Points can be awarded to potential candidate genes based on the number of independent researchers finding modulations within the hematological cancer as well as the number of different methods used to determine the expression level of these genes in the hematological cancer. The genes can then be ranked according to the number of points awarded, and those with the highest number of points may be selected as candidate genes. The number of candidate genes selected can vary depending on the number of features to be analyzed.

Gene sequences for genes of interest can be obtained from a variety of publicly available and proprietary sequence databases (including Genbank, dbEST, UniGene, and TIGR and SAGE databases) including sequences corresponding to expressed nucleotide sequences, such as expressed sequence tags (ESTs). For example, the Genbank™ website located at the NCBI website among others, can be readily accessed and searched via the internet. These and other sequence and clone database resources are currently available; however, a number of additional or alternative databases comprising gene sequences, EST sequences, clone repositories, PCR primer sequences, and the like corresponding to individual nucleotide sequences are also known and are suitable for the purposes of the invention.

While the above discussion relates primarily to “genomics” approaches, one skilled in the art will understand that numerous, analogous “proteomics” approaches are also suitable for selection of candidate genes for the HCP sets. For example, a differentially expressed protein product can, for example, be identified using Western analysis, two-dimensional gel analysis, chromatographic separation, mass spectrometric detection, protein-fusion reporter constructs, colorimetric assays, binding to a protein array, or by characterization of polysomal mRNA. The protein is further characterized and the nucleotide sequence encoding the protein is identified using standard techniques, e.g. by screening a cDNA library using a probe based on protein sequence information. Genes identified in this manner can also be included in the HCP set.

Representative, non-limiting examples of candidate genes for inclusion in HCP sets in accordance with the present invention are shown in Table 1.

TABLE 1
Candidate genes whose expression pattern is indicative of one or more
feature of a hematological cancer selected from the group of lymphoma
and leukemia

| GenBank | |
|---|---|
| Accession | Symbol |
| AA594161 | MYH11 |
| AA766908 | MME |
| AB014540 | SWAP70 |
| AF196185 | PARD3 |
| AI597616 | MRPL33 |
| AI634809 | ARID5B |
| AI672553 | AKAP12 |
| AI809213 | RGS13 |
| AK022231 | STAT1 |
| AK022293 | CTSD |
| AK055652 | C3orf6 |
| AL080130 | FLJ14001 |
| AL833316 | MIR |
| AW291384 | STS-1 |
| BE897089 | ZNF91 |
| BG993697 | SMYD3 |
| BI769730 | HLA-DRB1 |
| BQ632574 | RASA1 |
| BX504817 | SFRS7 |
| H53164 | IRF8 |
| H57732 | TGFBR2 |
| L29376 | 3.8-1 |
| M80899 | AHNAK |
| NM_000038 | APC |
| NM_000043 | FAS |
| NM_000061 | BTK |
| NM_000075 | CDK4 |
| NM_000109 | DMD |
| NM_000189 | HK2 |
| NM_000237 | LPL |
| NM_000269 | NME1 |
| NM_000271 | NPC1 |
| NM_000295 | SERPINA1 |
| NM_000313 | PROS1 |
| NM_000397 | CYBB |
| NM_000405 | GM2A |
| NM_000424 | KRT5 |
| NM_000483 | APOC2 |
| NM_000576 | IL1B |
| NM_000579 | CCR5 |
| NM_000584 | IL8 |
| NM_000585 | IL15 |
| NM_000600 | IL6 |
| NM_000626 | CD79B |
| NM_000627 | LTBP1 |
| NM_000633 | BCL2 |
| NM_000655 | SELL |
| NM_000713 | BLVRB |
| NM_000785 | CYP27B1 |
| NM_000788 | DCK |
| NM_000853 | GSTT1 |
| NM_000877 | IL1R1 |
| NM_000917 | P4HA1 |
| NM_000927 | ABCB1 |
| NM_001067 | TOP2A |
| NM_001068 | TOP2B |
| NM_001110 | ADAM10 |
| NM_001154 | ANXA5 |
| NM_001166 | BIRC2 |
| NM_001196 | BID |
| NM_001230 | CASP10 |
| NM_001238 | CCNE1 |
| NM_001242 | TNFRSF7 |
| NM_001254 | CDC6 |
| NM_001350 | DAXX |
| NM_001425 | EMP3 |
| NM_001552 | IGFBP4 |
| NM_001558 | IL10RA |
| NM_001618 | PARP1 |
| NM_001621 | AHR |
| NM_001647 | APOD |
| NM_001663 | ARF6 |
| NM_001706 | BCL6 |
| NM_001760 | CCND3 |
| NM_001762 | CCT6A |
| NM_001770 | CD19 |
| NM_001771 | CD22 |
| NM_001779 | CD58 |
| NM_001781 | CD69 |
| NM_001782 | CD72 |
| NM_001826 | CKS1B |
| NM_001827 | CKS2 |
| NM_001831 | CLU |
| NM_001852 | COL9A2 |
| NM_001853 | COL9A3 |
| NM_001877 | CR2 |
| NM_001888 | CRYM |
| NM_001894 | CSNK1E |
| NM_001951 | E2F5 |
| NM_001967 | EIF4A2 |
| NM_002002 | FCER2 |
| NM_002051 | GATA3 |
| NM_002095 | GTF2E2 |
| NM_002101 | GYPC |
| NM_002105 | H2AFX |
| NM_002120 | HLA-DOB |
| NM_002122 | HLA-DQA1 |
| NM_002155 | HSPA6 |
| NM_002189 | IL15RA |
| NM_002211 | ITGB1 |
| NM_002217 | ITIH3 |
| NM_002250 | KCNN4 |
| NM_002305 | LGALS1 |
| NM_002350 | LYN |
| NM_002358 | MAD2L1 |
| NM_002426 | MMP12 |
| NM_002467 | MYC |
| NM_002526 | NT5E |
| NM_002567 | PBP |
| NM_002646 | PIK3C2B |
| NM_002661 | PLCG2 |
| NM_002692 | POLE2 |
| NM_002738 | PRKCB1 |
| NM_002752 | MAPK9 |
| NM_002832 | PTPN7 |
| NM_002838 | PTPRC |
| NM_002891 | RASGRF1 |
| NM_002892 | ARID4A |
| NM_002943 | RORA |
| NM_002966 | S100A10 |
| NM_002967 | SAFB |
| NM_003037 | SLAMF1 |
| NM_003088 | FSCN1 |
| NM_003177 | SYK |
| NM_003217 | TEGT |
| NM_003226 | TFF3 |
| NM_003243 | TGFBR3 |
| NM_003254 | TIMP1 |
| NM_003290 | TPM4 |
| NM_003299 | TRA1 |
| NM_003332 | TYROBP |
| NM_003362 | UNG |
| NM_003451 | ZNF177 |
| NM_003648 | DGKD |
| NM_003656 | CAMK1 |
| NM_003810 | TNFSF10 |
| NM_003875 | GMPS |
| NM_003959 | HIP1R |
| NM_004049 | BCL2A1 |
| NM_004073 | PLK3 |
| NM_004091 | E2F2 |
| NM_004117 | FKBP5 |
| NM_004126 | GNG11 |
| NM_004184 | WARS |
| NM_004271 | LY86 |
| NM_004289 | NFE2L3 |
| NM_004322 | BAD |
| NM_004354 | CCNG2 |
| NM_004356 | CD81 |
| NM_004358 | CDC25B |
| NM_004422 | DVL2 |
| NM_004445 | EPHB6 |
| NM_004510 | SP110 |
| NM_004529 | MLLT3 |
| NM_004556 | NFKBIE |
| NM_004577 | PSPH |
| NM_004619 | TRAF5 |
| NM_004635 | MAPKAPK3 |
| NM_004737 | LARGE |
| NM_004844 | SH3BP5 |
| NM_004851 | NAPSA |
| NM_004951 | EBI2 |
| NM_005013 | NUCB2 |
| NM_005077 | TLE1 |
| NM_005127 | CLECSF2 |
| NM_005211 | CSF1R |
| NM_005213 | CSTA |
| NM_005214 | CTLA4 |
| NM_005271 | GLUD1 |
| NM_005292 | GPR18 |
| NM_005347 | HSPA5 |
| NM_005348 | HSPCA |
| NM_005415 | SLC20A1 |
| NM_005449 | TOSO |
| NM_005461 | MAFB |
| NM_005531 | IFI16 |
| NM_005582 | LY64 |
| NM_005601 | NKG7 |
| NM_005606 | LGMN |
| NM_005610 | RBBP4 |
| NM_005623 | CCL8 |
| NM_005638 | SYBL1 |
| NM_005652 | TERF2 |
| NM_005658 | TRAF1 |
| NM_005856 | RAMP3 |
| NM_005935 | MLLT2 |
| NM_005950 | MT1G |
| NM_005951 | MT1H |
| NM_006006 | ZBTB16 |
| NM_006014 | DXS9879E |
| NM_006115 | PRAME |
| NM_006162 | NFATC1 |
| NM_006164 | NFE2L2 |
| NM_006195 | PBX3 |
| NM_006218 | PIK3CA |
| NM_006254 | PRKCD |
| NM_006257 | PRKCQ |
| NM_006274 | CCL19 |
| NM_006317 | BASP1 |
| NM_006332 | IFI30 |
| NM_006475 | POSTN |
| NM_006498 | LGALS2 |
| NM_006558 | KHDRBS3 |
| NM_006620 | HBS1L |
| NM_006644 | HSPH1 |
| NM_006734 | HIVEP2 |
| NM_006761 | YWHAE |
| NM_006763 | BTG2 |
| NM_006841 | SLC38A3 |
| NM_006889 | CD86 |
| NM_006892 | DNMT3B |
| NM_006986 | MAGED1 |
| NM_007311 | BZRP |
| NM_007360 | KLRK1 |
| NM_007361 | NID2 |
| NM_012203 | GRHPR |
| NM_012448 | STAT5B |
| NM_012452 | TNFRSF13B |
| NM_013230 | CD24 |
| NM_014246 | CELSR1 |
| NM_014257 | CD209L |
| NM_014338 | PISD |
| NM_014456 | PDCD4 |
| NM_014686 | KIAA0355 |
| NM_014762 | DHCR24 |
| NM_015187 | KIAA0746 |
| NM_015641 | TES |
| NM_015866 | PRDM2 |
| NM_016076 | PNAS-4 |
| NM_016081 | KIAA0992 |
| NM_016187 | BIN2 |
| NM_016336 | UBE2J1 |
| NM_016628 | WAC |
| NM_017530 | LOC55565 |
| NM_017784 | OSBPL10 |
| NM_017935 | BANK1 |
| NM_017955 | CDCA4 |
| NM_018442 | IQWD1 |
| NM_018842 | BAIAP2L1 |
| NM_020151 | STARD7 |
| NM_020199 | C5orf15 |
| NM_020529 | NFKBIA |
| NM_020992 | PDLIM1 |
| NM_021813 | BACH2 |
| NM_021822 | APOBEC3G |
| NM_021950 | MS4A1 |
| NM_021966 | TCL1A |
| NM_022552 | DNMT3A |
| NM_023037 | 13CDNA73 |
| NM_024708 | ASB7 |
| NM_025113 | C13orf18 |
| NM_025216 | WNT10A |
| NM_030666 | SERPINB1 |
| NM_030764 | SPAP1 |
| NM_030775 | WNT5B |
| NM_031305 | ARHGAP24 |
| NM_031942 | CDCA7 |
| NM_032991 | CASP3 |
| NM_033208 | TIGD7 |
| NM_033274 | ADAM19 |
| NM_057735 | CCNE2 |
| NM_078467 | CDKN1A |
| NM_079421 | CDKN2D |
| NM_080593 | HIST1H2BK |
| NM_138340 | ABHD3 |
| NM_138379 | TIMD4 |
| NM_139207 | NAP1L1 |
| NM_139276 | STAT3 |
| NM_144646 | IGJ |
| NM_145804 | ABTB2 |
| NM_152785 | GCET2 |
| NM_172373 | ELF1 |
| NM_173624 | FLJ40504 |
| NM_175870 | LOC90925 |
| NM_177968 | PPM1B |
| NM_181443 | BTBD3 |
| NM_181838 | UBE2D2 |
| NM_182746 | MCM4 |
| NM_182776 | MCM7 |
| NM_198833 | SERPINB8 |
| NM_201593 | CACNB2 |
| R99527 | MGC39372 |
| T66903 | LOC54103 |
| U37028 | ITGAD |
| W56129 | SFRS9 |
| X58529 | IGHM |
| AA188785 | LOC286025 |
| AA347638 | BCR |
| AA601122 | SECTM1 |
| AA642467 | SLC2A14 |
| AA682722 | LARP |
| AA757235 | LOC399933 |
| AA765313 | FZD3 |
| AB002310 | HUWE1 |
| AB002333 | ZNF518 |
| AB024402 | ING1 |
| AB028952 | SYNPO |
| AB032991 | NDFIP2 |
| AF008915 | EVI5 |
| AF108138 | C15orf20 |
| AF116668 | LMO2 |
| AF143327 | FLJ32810 |
| AF200348 | D2S448 |
| AF245480 | MONDOA |
| AI075159 | SPTA1 |
| BU948323 | TCF4 |
| AI523104 | LILRB1 |
| AI675029 | C7 |
| AI694012 | PSMA5 |
| AI732248 | COL4A1 |
| AJ007509 | HNRPUL1 |
| AK022224 | ACTN1 |
| AK023928 | PSCD1 |
| AK024580 | NRP1 |
| AK025005 | MARCH1 |
| AK091853 | COL3A1 |
| AK126650 | CAPZB |
| AK127124 | ARHGEF4 |
| AK127132 | DRG1 |
| AK129940 | GNAI1 |
| AL049313 | CLIC5 |
| AL049327 | CBLB |
| AL049449 | GAB1 |
| AL050391 | CASP4 |
| AL137527 | LRCH3 |
| AL353942 | SEPT11 |
| AL833915 | TDRD9 |
| AL834278 | USP30 |
| AU252485 | MAP7 |
| AV650179 | PRG1 |
| AV686223 | KIAA1407 |
| AW014009 | MAP2K3 |
| AW190768 | SLC13A3 |
| AW292143 | GFAP |
| AW439398 | CCND1 |
| AW452039 | HRMT1L3 |
| AW615076 | LOC492304 |
| AW664012 | ALOX5 |
| AW967479 | CD38 |
| AW970654 | PHTF2 |
| BC028066 | NALP1 |
| BC034289 | LOC87769 |
| BC042366 | PCDH9 |
| BC045532 | LSM8 |
| BC050383 | TMPO |
| BC050602 | HIST1H2AC |
| BC058855 | VEGF |
| BE147267 | ECE1 |
| BE549606 | BZW1 |
| BE798965 | LAMC1 |
| BE927772 | SFRS3 |
| BF055235 | RIPK1 |
| BF513638 | WNT3 |
| BF848289 | CSDA |
| BG682263 | HDAC3 |
| BG683220 | PMS2 |
| BG747999 | MT1L |
| BI493513 | PHF8 |
| BI559738 | GPR56 |
| BM709325 | BCL7A |
| BM711190 | MED28 |
| BM727177 | GOLT1B |
| BQ009245 | GPM6B |
| BQ021469 | AGPS |
| BQ215285 | LEF1 |
| BQ898221 | ITGA6 |
| BU929213 | SESN3 |
| BU954396 | RPL11 |
| BX095432 | CNTN5 |
| BX640908 | EVI1 |
| BX648339 | USP53 |
| BX648365 | SEPT7 |
| BX649193 | TKT |
| C05886 | ETV6 |
| D31762 | TRAM2 |
| D86967 | EDEM1 |
| H41942 | NPEPPS |
| M60028 | HLA-DQB1 |
| M77810 | GATA2 |
| N26272 | MSI2 |
| N28431 | COL6A1 |
| N41620 | WWP1 |
| N53163 | COL27A1 |
| N54946 | EWSR1 |
| N70000 | UBE3B |
| NM_000016 | ACADM |
| NM_000022 | ADA |
| NM_000024 | ADRB2 |
| NM_000033 | ABCD1 |
| NM_000062 | SERPING1 |
| NM_000089 | COL1A2 |
| NM_000099 | CST3 |
| NM_000110 | DPYD |
| NM_000118 | ENG |
| NM_000120 | EPHX1 |
| NM_000147 | FUCA1 |
| NM_000169 | GLA |
| NM_000182 | HADHA |
| NM_000194 | HPRT1 |
| NM_000214 | JAG1 |
| NM_000234 | LIG1 |
| NM_000239 | LYZ |
| NM_000246 | MHC2TA |
| NM_000249 | MLH1 |
| NM_000251 | MSH2 |
| NM_000277 | PAH |
| NM_000305 | PON2 |
| NM_000314 | PTEN |
| NM_000341 | SLC3A1 |
| NM_000361 | THBD |
| NM_000382 | ALDH3A2 |
| NM_000395 | CSF2RB |
| NM_000417 | IL2RA |
| NM_000418 | IL4R |
| NM_000435 | NOTCH3 |
| NM_000448 | RAG1 |
| NM_000484 | APP |
| NM_000485 | APRT |
| NM_000536 | RAG2 |
| NM_000545 | TCF1 |
| NM_000546 | TP53 |
| NM_000561 | GSTM1 |
| NM_000574 | DAF |
| NM_000578 | SLC11A1 |
| NM_000594 | TNF |
| NM_000598 | IGFBP3 |
| NM_000601 | HGF |
| NM_000604 | FGFR1 |
| NM_000610 | CD44 |
| NM_000628 | IL10RB |
| NM_000629 | IFNAR1 |
| NM_000640 | IL13RA2 |
| NM_000660 | TGFB1 |
| NM_000700 | ANXA1 |
| NM_000701 | ATP1A1 |
| NM_000717 | CA4 |
| NM_00729 | CCK |
| NM_000732 | CD3D |
| NM_000773 | CYP2E1 |
| NM_000778 | CYP4N11 |
| NM_000791 | DHFR |
| NM_000801 | FKBP1A |
| NM_000852 | GSTP1 |
| NM_000856 | GUCY1A3 |
| NM_000867 | HTR2B |
| NM_000878 | IL2RB |
| NM_000885 | ITGA4 |
| NM_000887 | ITGAX |
| NM_000905 | NPY |
| NM_000906 | NPR1 |
| NM_000953 | PTGDR |
| NM_000960 | PTGDR |
| NM_000964 | RARA |
| NM_000967 | RPL3 |
| NM_000968 | RPL4 |
| NM_000969 | RPL5 |
| NM_000970 | RPL6 |
| NM_000972 | RPL7A |
| NM_001001 | CD36 |
| 547 | |
| NM_001005 | RPS3 |
| NM_001009 | RPS5 |
| NM_001015 | RPS11 |
| NM_001019 | RPS15A |
| NM_001069 | TUBB2 |
| NM_001079 | ZAP70 |
| NM_001092 | ABR |
| NM_001130 | AES |
| NM_001150 | ANPEP |
| NM_001165 | BIRC3 |
| NM_001175 | ARHGDIB |
| NM_001178 | ARNTL |
| NM_001207 | BTF3 |
| NM_001211 | BUB1B |
| NM_001219 | CALU |
| NM_001226 | CASP6 |
| NM_001228 | CASP8 |
| NM_001229 | CASP9 |
| NM_001237 | CCNA2 |
| NM_001239 | CCNH |
| NM_001255 | CDC20 |
| NM_001256 | CDC27 |
| NM_001265 | CDX2 |
| NM_001266 | CES1 |
| NM_001273 | CHD4 |
| NM_001274 | CHEK1 |
| NM_001280 | CIRBP |
| NM_001295 | CCR1 |
| NM_001312 | CRIP2 |
| NM_001313 | CRMP1 |
| NM_001337 | CX3CR1 |
| NM_001344 | DAD1 |
| NM_001345 | DGKA |
| NM_001398 | ECH1 |
| NM_001404 | EEF1G |
| NM_001428 | ENO1 |
| NM_001436 | FBL |
| NM_001441 | FAAH |
| NM_001456 | FLNA |
| NM_001459 | FLT3LG |
| NM_001462 | FPRL1 |
| NM_001489 | NR6A1 |
| NM_001513 | GSTZ1 |
| NM_001515 | GTF2H2 |
| NM_001540 | HSPB1 |
| NM_001560 | IL13RA1 |
| NM_001607 | ACAA1 |
| NM_001630 | ANXA8 |
| NM_001640 | APEH |
| NM_001654 | ARAF |
| NM_001664 | RHOA |
| NM_001700 | AZU1 |
| NM_001712 | CEACAM1 |
| NM_001715 | BLK |
| NM_001716 | BLR1 |
| NM_001728 | BSG |
| NM_001734 | C1S |
| NM_001743 | CALM2 |
| NM_001747 | CAPG |
| NM_001752 | CAT |
| NM_001753 | CAV1 |
| NM_001759 | CCND2 |
| NM_001767 | CD2 |
| NM_001772 | CD33 |
| NM_001774 | CD37 |
| NM_001776 | ENTPD1 |
| NM_001783 | CD79A |
| NM_001786 | CDC2 |
| NM_001793 | CDH3 |
| NM_001794 | CDH4 |
| NM_001795 | CDH5 |
| NM_001798 | CDK2 |
| NM_001830 | CLCN4 |
| NM_001838 | CCR7 |
| NM_001839 | CNN3 |
| NM_001897 | CSPG4 |
| NM_001901 | CTGF |
| NM_001905 | CTPS |
| NM_001908 | CTSB |
| NM_001916 | CYC1 |
| NM_001923 | DDB1 |
| NM_001928 | DF |
| NM_001939 | DRP2 |
| NM_001941 | DSC3 |
| NM_001952 | E2F6 |
| NM_001964 | EGR1 |
| NM_001993 | F3 |
| NM_001999 | FBN2 |
| NM_002015 | FOXO1A |
| NM_002017 | FLI1 |
| NM_002023 | FMOD |
| NM_002033 | FUT4 |
| NM_002046 | GAPD |
| NM_002049 | GATA1 |
| NM_002064 | GLRX |
| NM_002065 | GLUL |
| NM_002067 | GNA11 |
| NM_002068 | GNA15 |
| NM_002076 | GNS |
| NM_002086 | GRB2 |
| NM_002090 | CXCL3 |
| NM_002094 | GSPT1 |
| NM_002102 | GYPE |
| NM_002107 | H3F3A |
| NM_002108 | HAL |
| NM_002110 | HCK |
| NM_002118 | HLA-DMB |
| NM_002121 | HLA-DPB1 |
| NM_002128 | HMGB1 |
| NM_002129 | HMGB2 |
| NM_002135 | NR4A1 |
| NM_002141 | HOXA4 |
| NM_002145 | HOXB2 |
| NM_002162 | ICAM3 |
| NM_002166 | ID2 |
| NM_002167 | ID3 |
| NM_002186 | IL9R |
| NM_002193 | INHBB |
| NM_002198 | IRF1 |
| NM_002199 | IRF2 |
| NM_002222 | ITPR1 |
| NM_002224 | ITPR3 |
| NM_002228 | JUN |
| NM_002229 | JUNB |
| NM_002230 | JUP |
| NM_002248 | KCNN1 |
| NM_002276 | KRT19 |
| NM_002295 | LAMR1 |
| NM_002312 | LIG4 |
| NM_002341 | LTB |
| NM_002349 | LY75 |
| NM_002351 | SH2D1A |
| NM_002353 | TACSTD2 |
| NM_002356 | MARCKS |
| NM_002379 | MATN1 |
| NM_002388 | MCM3 |
| NM_002390 | ADAM11 |
| NM_002392 | MDM2 |
| NM_002393 | MDM4 |
| NM_002398 | MEIS1 |
| NM_002412 | MGMT |
| NM_002414 | CD99 |
| NM_002415 | MIF |
| NM_002417 | MKI67 |
| NM_002419 | MAP3K11 |
| NM_002421 | MMP1 |
| NM_002422 | MMP3 |
| NM_002436 | MPP1 |
| NM_002441 | MSH5 |
| NM_002444 | MSN |
| NM_002447 | MST1R |
| NM_002460 | IRF4 |
| NM_002462 | MX1 |
| NM_002466 | MYBL2 |
| NM_002475 | MLC1SA |
| NM_002483 | CEACAM6 |
| NM_002512 | NME2 |
| NM_002514 | NOV |
| NM_002527 | NTF3 |
| NM_002530 | NTRK3 |
| NM_002572 | PAFAH1B2 |
| NM_002576 | PAK1 |
| NM_002585 | PBX1 |
| NM_002592 | PCNA |
| NM_002600 | PDE4B |
| NM_002608 | PDGFB |
| NM_002609 | PDGFRB |
| NM_002616 | PER1 |
| NM_002619 | PF4 |
| NM_002621 | PFC |
| NM_002648 | PIM1 |
| NM_002658 | PLAU |
| NM_002659 | PLAUR |
| NM_002687 | PNN |
| NM_002704 | PPBP |
| NM_002729 | HHEX |
| NM_002730 | PRKACA |
| NM_002744 | PRKCZ |
| NM_002753 | MAPK10 |
| NM_002758 | MAP2K6 |
| NM_002791 | PSMA6 |
| NM_002800 | PSMB9 |
| NM_002814 | PSMD10 |
| NM_002827 | PTPN1 |
| NM_002831 | PTPN6 |
| NM_002835 | PTPN12 |
| NM_002844 | PTPRK |
| NM_002856 | PVRL2 |
| NM_002868 | RAB5B |
| NM_002870 | RAB13 |
| NM_002880 | RAF1 |
| NM_002887 | RARS |
| NM_002893 | RBBP7 |
| NM_002901 | RCN1 |
| NM_002908 | REL |
| NM_002913 | RFC1 |
| NM_002934 | RNASE2 |
| NM_002935 | RNASE3 |
| NM_002936 | RNASEH1 |
| NM_002953 | RPS6KA1 |
| NM_002960 | S100A3 |
| NM_002961 | S100A4 |
| NM_002971 | SATB1 |
| NM_002972 | SBF1 |
| NM_002975 | CLEC11A |
| NM_002983 | CCL3 |
| NM_002984 | CCL4 |
| NM_002985 | CCL5 |
| NM_003002 | SDHD |
| NM_003005 | SELP |
| NM_003006 | SELPLG |
| NM_003009 | SEPW1 |
| NM_003025 | SH3GL1 |
| NM_003038 | SLC1A4 |
| NM_003039 | SLC2A5 |
| NM_003058 | SLC22A2 |
| NM_003072 | SMARCA4 |
| NM_003073 | SMARCB1 |
| NM_003100 | SNX2 |
| NM_003113 | SP100 |
| NM_003118 | SPARC |
| NM_003120 | SPI1 |
| NM_003121 | SPIB |
| NM_003123 | SPN |
| NM_003127 | SPTAN1 |
| NM_003128 | SPTBN1 |
| NM_003133 | SRP9 |
| NM_003159 | CDKL5 |
| NM_003189 | TAL1 |
| NM_003200 | TCF3 |
| NM_003222 | TFAP2C |
| NM_003225 | TFF1 |
| NM_003234 | TFRC |
| NM_003262 | TLOC1 |
| NM_003288 | TPD52L2 |
| NM_003302 | TRIP6 |
| NM_003311 | PHLDA2 |
| NM_003315 | DNAJC7 |
| NM_003318 | TTK |
| NM_003352 | SUMO1 |
| NM_003361 | UMOD |
| NM_003383 | VLDLR |
| NM_003390 | WEE1 |
| NM_003395 | WNT9A |
| NM_003403 | YY1 |
| NM_003415 | ZNF268 |
| NM_003425 | ZNF45 |
| NM_003427 | ZNF76 |
| NM_003453 | ZNF198 |
| NM_003467 | CXCR4 |
| NM_003480 | MEAP5 |
| NM_003549 | HYAL3 |
| NM_003582 | DYRK3 |
| NM_003627 | SLC43A1 |
| NM_003636 | KCNAB2 |
| NM_003645 | SLC27A2 |
| NM_003692 | TMEFF1 |
| NM_003701 | TNESF11 |
| NM_003707 | RUVBL1 |
| NM_003739 | AKR1C3 |
| NM_003746 | DNCL1 |
| NM_003753 | EIF3S7 |
| NM_003801 | GPAA1 |
| NM_003805 | CRADD |
| NM_003821 | RIPK2 |
| NM_003824 | FADD |
| NM_003842 | TNFRSF10B |
| NM_003852 | TIF1 |
| NM_003874 | CD84 |
| NM_003877 | SOCS2 |
| NM_003879 | CFLAR |
| NM_003884 | PCAF |
| NM_003900 | SQSTM1 |
| NM_003902 | FUBP1 |
| NM_003914 | CCNA1 |
| NM_003954 | MAP3K14 |
| NM_003955 | SOCS3 |
| NM_003974 | DOK2 |
| NM_003998 | NFKB1 |
| NM_004039 | ANXA2 |
| NM_004047 | ATP6V0B |
| NM_004064 | CDKN1B |
| NM_004067 | CHN2 |
| NM_004077 | CS |
| NM_004083 | DDIT3 |
| NM_004088 | DNTT |
| NM_004093 | EFNB2 |
| NM_004099 | STOM |
| NM_004104 | FASN |
| NM_004111 | FEN1 |
| NM_004119 | FLT3 |
| NM_004177 | STX3A |
| NM_004208 | PDCD8 |
| NM_004221 | NK4 |
| NM_004235 | KLF4 |
| NM_004310 | RHOH |
| NM_004313 | ARRB2 |
| NM_004320 | ATP2A1 |
| NM_004324 | BAX |
| NM_004335 | BST2 |
| NM_004336 | BUB1 |
| NM_004343 | CALR |
| NM_004355 | CD74 |
| NM_004374 | COX6C |
| NM_004380 | CREBBP |
| NM_004385 | CSPG2 |
| NM_004413 | DPEP1 |
| NM_004425 | ECM1 |
| NM_004430 | EGR3 |
| NM_004448 | ERBB2 |
| NM_004449 | ERG |
| NM_004454 | ETV5 |
| NM_004475 | FLOT2 |
| NM_004494 | HDGF |
| NM_004501 | HNRPU |
| NM_004508 | IDI1 |
| NM_004513 | IL16 |
| NM_004526 | MCM2 |
| NM_004530 | MMP2 |
| NM_004557 | NOTCH4 |
| NM_004583 | RAB5C |
| NM_004615 | TM4SF2 |
| NM_004624 | VIPR1 |
| NM_004633 | IL1R2 |
| NM_004642 | CDK2AP1 |
| NM_004688 | NMI |
| NM_004689 | MTA1 |
| NM_004706 | ARHGEF1 |
| NM_004725 | BUB3 |
| NM_004815 | PARG1 |
| NM_004827 | ABCG2 |
| NM_004867 | ITM2A |
| NM_004924 | ACTN4 |
| NM_004936 | CDKN2B |
| NM_004938 | DAPK1 |
| NM_004941 | DHX8 |
| NM_004972 | JAK2 |
| NM_004994 | MMP9 |
| NM_004997 | MYBPH |
| NM_005012 | ROR1 |
| NM_005080 | XBP1 |
| NM_005084 | PLA2G7 |
| NM_005085 | NUP214 |
| NM_005098 | MSC |
| NM_005104 | BRD2 |
| NM_005123 | NR1H4 |
| NM_005157 | ABL1 |
| NM_005161 | AGTRL1 |
| NM_005178 | BCL3 |
| NM_005187 | CBFA2T3 |
| NM_005192 | CDKN3 |
| NM_005197 | CHES1 |
| NM_005215 | DCC |
| NM_005225 | E2F1 |
| NM_005230 | ELK3 |
| NM_005239 | ETS2 |
| NM_005245 | FAT |
| NM_005246 | FER |
| NM_005248 | FGR |
| NM_005252 | FOS |
| NM_005263 | GFI1 |
| NM_005317 | GZMM |
| NM_005318 | H1F0 |
| NM_005334 | HCFC1 |
| NM_005335 | HCLS1 |
| NM_005339 | HIP2 |
| NM_005340 | HINT1 |
| NM_005356 | LCK |
| NM_005375 | MYB |
| NM_005385 | NKTR |
| NM_005424 | TIE1 |
| NM_005427 | TP73 |
| NM_005439 | MLF2 |
| NM_005494 | DNAJB6 |
| NM_005504 | BCAT1 |
| NM_005521 | TLX1 |
| NM_005529 | HSPG2 |
| NM_005533 | IFI35 |
| NM_005556 | KRT7 |
| NM_005563 | STMN1 |
| NM_005566 | LDHA |
| NM_005572 | LMNA |
| NM_005583 | LYL1 |
| NM_005589 | ALDH6A1 |
| NM_005608 | PTPRCAP |
| NM_005621 | S100A12 |
| NM_005651 | TDO2 |
| NM_005654 | NR2F1 |
| NM_005671 | D8S2298E |
| NM_005687 | FARSLB |
| NM_005730 | CTDSP2 |
| NM_005731 | ARPC2 |
| NM_005738 | ARL4A |
| NM_005761 | PLXNC1 |
| NM_005767 | P2RY5 |
| NM_005781 | TNK2 |
| NM_005794 | DHRS2 |
| NM_005808 | CTDSPL |
| NM_005809 | PRDX2 |
| NM_005826 | HNRPR |
| NM_005835 | SLC17A2 |
| NM_005845 | ABCC4 |
| NM_005870 | SAP18 |
| NM_005895 | GOLGA3 |
| NM_005900 | SMAD1 |
| NM_005902 | SMAD3 |
| NM_005907 | MAN1A1 |
| NM_005911 | MAT2A |
| NM_005923 | MAP3K5 |
| NM_005933 | MLL |
| NM_005936 | MLLT4 |
| NM_005944 | CD200 |
| NM_005954 | MT3 |
| NM_005956 | MTHFD1 |
| NM_005957 | MTHFR |
| NM_005965 | MYLK |
| NM_006005 | WFS1 |
| NM_006017 | PROM1 |
| NM_006038 | SPATA2 |
| NM_006060 | ZNFN1A1 |
| NM_006079 | CITED2 |
| NM_006082 | K-APHA-1 |
| NM_006084 | ISGF3G |
| NM_006098 | GNB2L1 |
| NM_006101 | KNTC2 |
| NM_006113 | VAV3 |
| NM_006120 | HLA-DMA |
| NM_006142 | SFN |
| NM_006152 | LRMP |
| NM_006184 | NUCB1 |
| NM_006185 | NUMA1 |
| NM_006187 | OAS3 |
| NM_006190 | ORC2L |
| NM_006191 | PA2G4 |
| NM_006196 | PCBP1 |
| NM_006209 | ENPP2 |
| NM_006216 | SERPINE2 |
| NM_006230 | POLD2 |
| NM_006231 | POLE |
| NM_006235 | POU2AF1 |
| NM_006255 | PRKCH |
| NM_006263 | PSME1 |
| NM_006268 | DPF2 |
| NM_006273 | CCL7 |
| NM_006286 | TFDP2 |
| NM_006299 | ZNF193 |
| NM_006301 | MAP3K12 |
| NM_006302 | GCS1 |
| NM_006325 | RAN |
| NM_006329 | FBLN5 |
| NM_006343 | MERTK |
| NM_006354 | TADA3L |
| NM_006355 | TRIM38 |
| NM_006362 | NXF1 |
| NM_006427 | SIVA |
| NM_006472 | TXNIP |
| NM_006495 | EVI2B |
| NM_006509 | RELB |
| NM_006522 | WNT6 |
| NM_006534 | NCOA3 |
| NM_006559 | KHDRBS1 |
| NM_006565 | CTCF |
| NM_006579 | EBP |
| NM_006591 | POLD3 |
| NM_006602 | TCFL5 |
| NM_006676 | USP20 |
| NM_006739 | MCM5 |
| NM_006747 | SIPA1 |
| NM_006748 | SLA |
| NM_006759 | UGP2 |
| NM_006762 | LAPTM5 |
| NM_006769 | LMO4 |
| NM_006803 | AP3M2 |
| NM_006820 | IFI44L |
| NM_006825 | CKAP4 |
| NM_006845 | KIF2C |
| NM_006857 | RY1 |
| NM_006875 | PIM2 |
| NM_006904 | PRKDC |
| NM_006910 | RBBP6 |
| NM_006931 | SLC2A3 |
| NM_006932 | SMTN |
| NM_006937 | SUMO2 |
| NM_006938 | SNRPD1 |
| NM_006963 | ZNF22 |
| NM_006981 | NR4A3 |
| NM_006994 | BTN3A3 |
| NM_007022 | CYB561D2 |
| NM_007063 | TBC1D8 |
| NM_007065 | CDC37 |
| NM_007079 | PTP4A3 |
| NM_007111 | TFDP1 |
| NM_007117 | TRH |
| NM_012092 | ICOS |
| NM_012120 | CD2AP |
| NM_012207 | HNRPH3 |
| NM_012223 | MYO1B |
| NM_012267 | HSPBP1 |
| NM_012317 | LDOC1 |
| NM_012323 | MAFF |
| NM_012334 | MYO10 |
| NM_012384 | GMEB2 |
| NM_012417 | PITPNC1 |
| NM_012423 | RPL13A |
| NM_013282 | UHRF1 |
| NM_013314 | BLNK |
| NM_013416 | NCF4 |
| NM_014005 | PCDHA6 |
| NM_014207 | CD5 |
| NM_014232 | VAMP2 |
| NM_014244 | ADAMTS2 |
| NM_014298 | QPRT |
| NM_014345 | ZNF318 |
| NM_014365 | HSPB8 |
| NM_014575 | SCHIP1 |
| NM_014595 | NT5C |
| NM_014614 | PSME4 |
| NM_014624 | S100A6 |
| NM_014667 | VGLL4 |
| NM_014707 | HDAC9 |
| NM_014724 | ZNF305 |
| NM_014734 | KIAA0247 |
| NM_014735 | PHF16 |
| NM_014780 | CUL7 |
| NM_014792 | KIAA0125 |
| NM_014824 | FCHSD2 |
| NM_014890 | DOC1 |
| NM_014899 | RHOBTB3 |
| NM_014914 | CENTG2 |
| NM_015050 | KIAA0082 |
| NM_015069 | ZNE423 |
| NM_015099 | CAMTA2 |
| NM_015147 | KIAA0582 |
| NM_015149 | RGL1 |
| NM_015158 | ANKRD15 |
| NM_015166 | MLC1 |
| NM_015196 | KIAA0922 |
| NM_015210 | KIAA0802 |
| NM_015261 | KIAA0056 |
| NM_015335 | THRAP2 |
| NM_015436 | RCHY1 |
| NM_015458 | MTMR9 |
| NM_015474 | SAMHD1 |
| NM_015528 | RNF167 |
| NM_015568 | PPP1R16B |
| NM_015570 | AUTS2 |
| NM_015636 | ELF2B4 |
| NM_015670 | SENP3 |
| NM_015710 | GLTSCR2 |
| NM_015833 | ADARB1 |
| NM_015855 | WIT-1 |
| NM_015869 | PPARG |
| NM_015965 | GRIM19 |
| NM_016041 | DERL2 |
| NM_016091 | EIF3S6IP |
| NM_016184 | CLECSF6 |
| NM_016195 | MPHOSPH1 |
| NM_016221 | DCTN4 |
| NM_016308 | UMP-CMPK |
| NM_016343 | CENPF |
| NM_016570 | PTX1 |
| NM_016734 | PAX5 |
| NM_017437 | CPSF2 |
| NM_017448 | LDHC |
| NM_017617 | NOTCH1 |
| NM_017771 | PXK |
| NM_017787 | C10orf26 |
| NM_017794 | KIAA1797 |
| NM_018136 | ASPM |
| NM_018209 | ARFGAP1 |
| NM_018248 | FLJ10858 |
| NM_018462 | C3orf10 |
| NM_018664 | SNFT |
| NM_018951 | HOXA10 |
| NM_019028 | ZDHHC13 |
| NM_019071 | ING3 |
| NM_019102 | HOXA5 |
| NM_019111 | HLA-DRA |
| NM_019841 | TRPV5 |
| NM_019846 | CCL28 |
| NM_019857 | CTPS2 |
| NM_020310 | MNT |
| NM_020368 | SAS10 |
| NM_020371 | AVEN |
| NM_020631 | KIAA0720 |
| NM_020657 | ZNF304 |
| NM_020841 | OSBPL8 |
| NM_020843 | ZNF291 |
| NM_020892 | DTX2 |
| NM_020944 | GBA2 |
| NM_020956 | PRX |
| NM_020998 | MST1 |
| NM_021019 | MYL6 |
| NM_021025 | TLX3 |
| NM_021038 | MBNL1 |
| NM_021071 | DO |
| NM_021103 | TMSB10 |
| NM_021111 | RECK |
| NM_021114 | SPINK2 |
| NM_021603 | FXYD2 |
| NM_021643 | TRIB2 |
| NM_021814 | ELOVL5 |
| NM_021906 | USP9X |
| NM_021949 | ATP2B3 |
| NM_021960 | MCL1 |
| NM_022067 | C14orf133 |
| NM_022127 | SLC28A3 |
| NM_022161 | BIRC7 |
| NM_022334 | ITGB1BP1 |
| NM_022366 | TFB2M |
| NM_022436 | ABCG5 |
| NM_022438 | MAL |
| NM_022469 | GREM2 |
| NM_022549 | FEZ1 |
| NM_022716 | PRRX1 |
| NM_024006 | VKORC1 |
| NM_024099 | MGC2477 |
| NM_024319 | C1orf35 |
| NM_024408 | NOTCH2 |
| NM_024424 | WT1 |
| NM_024713 | C15orf29 |
| NM_024728 | C7orf10 |
| NM_025263 | PRR3 |
| NM_030660 | ATXN3 |
| NM_030674 | SLC38A1 |
| NM_030915 | LBH |
| NM_030926 | ITM2C |
| NM_030935 | THG-1 |
| NM_031243 | HNRPA2B1 |
| NM_031423 | CDCA1 |
| NM_031966 | CCNB1 |
| NM_032121 | DKFZp564K142 |
| NM_033238 | PML |
| NM_033512 | TSPYL5 |
| NM_033554 | HLA-DPA1 |
| NM_033642 | FGF13 |
| NM_057168 | WNT16 |
| NM_058195 | CDKN2A |
| NM_080284 | ABCA6 |
| NM_080700 | TREX2 |
| NM_130439 | MXI1 |
| NM_133259 | LRPPRC |
| NM_133378 | TTN |
| NM_138714 | NFAT5 |
| NM_144578 | C14orf32 |
| NM_144628 | TBC1D20 |
| NM_145698 | ACBD5 |
| NM_145867 | LTC4S |
| NM_147180 | PPP3R2 |
| NM_152739 | HOXA9 |
| NM_152788 | EB-1 |
| NM_152827 | SNX3 |
| NM_152890 | COL24A1 |
| NM_156038 | CSF3R |
| NM_172020 | POM121 |
| NM_173156 | C1orf16 |
| NM_173216 | SIAT1 |
| NM_173609 | C15orf21 |
| NM_173852 | KRTCAP2 |
| NM_175739 | SERPINA9 |
| NM_175744 | RHOC |
| NM_178586 | PPP2R5C |
| NM_181311 | TAZ |
| NM_181336 | LEMD2 |
| NM_181339 | IL24 |
| NM_181430 | FOXK2 |
| NM_181493 | ITPA |
| NM_181702 | GEM |
| NM_182729 | TXNRD1 |
| NM_182810 | ATF4 |
| NM_183395 | CIAS1 |
| NM_198232 | RNASE1 |
| NM_198400 | NEDD4 |
| NM_199335 | FYB |
| NM_199423 | WWP2 |
| NM_201592 | GPM6A |
| NM_201632 | TCF7 |
| NM_201998 | SF1 |
| NM_212474 | FN1 |
| R14777 | CYFIP2 |
| R56397 | PIK3C3 |
| R59027 | GRP58 |
| R72151 | GNL3 |
| R79128 | MAP3K1 |
| R86893 | C6orf110 |
| T07281 | NRIP1 |
| T28925 | ITGAL |
| U60115 | FHL1 |
| U79271 | AKT3 |
| W04885 | MYL4 |
| X66087 | MYBL1 |
| AK024272 | |
| AK025231 | IGLC2 |
| AL080190 | |
| BU618233 | |
| NM_001353.4 | AKR1C1 |

Accordingly, one embodiment of the present invention provides for a library of candidate genes suitable for profiling hematological cancers. In a further embodiment, the library of candidate genes comprises the genes set forth in Table 1. The library provides a resource from which genes appropriate for inclusion in a HCP sets can be selected.

Once candidate genes have been identified, an HCP set is formed by selecting those genes relating to the hematological cancer of interest that are indicative of features of the hematological cancer that are to be investigated. If more than one hematological cancer is to be investigated, or more than one sub-type of a hematological cancer is to be investigated, then different HCP sets can be formed containing genes that are indicative of the feature(s) of interest. For example if lymphoma and leukemia are to be investigated, then different HCP sets can be created, each one relating to a sub-type of lymphoma or a sub-type of leukemia and containing genes that are indicative of the feature(s) of interest.

It will be readily apparent that some genes are suitable for inclusion in more than one HCP set. For example, a gene that allows two lymphomas to be distinguished, such as DLBCL and FL, would be suitable for inclusion in an HCP set relating to DLBCL as well as an HCP set relating to FL. Alternatively, a gene that allows two leukemias to be distinguished, for example CLL and AML, would be suitable for inclusion in an HCP set relating to CLL as well as an HCP set relating to AML. Examples of genes that may be included in more than one HCP set are: AKR1C1, MAL (T-cell differentiation protein), and TIMP1.

The HCP set can comprise between one and about 2000 genes, depending on the number of features of the lymphoma the set is intended to represent. When the set comprises more than one gene, each gene of the set can relate to a different feature of the hematological cancer, or multiple genes within the HCP set can relate to the same feature. Thus, the HCP set can comprise genes that are indicative of one feature of a hematological cancer, genes that are indicative of more than one feature of a hematological cancer, or combinations thereof.

In one embodiment, the HCP set comprises at least 5 genes. In another embodiment, the HCP set comprises at least 10 genes. In a further embodiment, the HCP set comprises at least 15 genes. In other embodiments, the HCP set comprises at least 20, at least 25, at least 30, at least 35, and at least 40 genes. As indicated above, the HCP set typically comprises less than 2000 genes. In one embodiment of the present invention, therefore, the HCP set comprises between about 5 and about 2000 genes. In another embodiment, the HCP set comprises between about 5 and about 1500 genes. In a further embodiment, the HCP set comprises between about 5 and about 1000 genes. In other embodiments, the HCP set comprises between about 5 and about 750 genes, between about 5 and about 500 genes, between about 5 and about 400 genes, and between about 5 and about 300 genes. In other embodiments, the HCP set comprises between about 10 and about 1500 genes, between about 15 and about 1000 genes, between about 20 and about 750 genes, between about 25 and about 500 genes, between about 30 and about 400 genes, between about 30 and about 300 genes, and between about 30 and about 250 genes.

As indicated above, the HCP set is representative of at least one feature of a hematological cancer. In one embodiment of the present invention, the HCP set is representative of two or more features of a specific hematological cancer. In another embodiment, the HCP set is representative of between one and 20 features of a specific hematological cancer. In a further embodiment, the HCP set is representative of between 2 and 20 features of a specific hematological cancer. In yet another embodiment, the HCP set is representative of between 3 and 20 features of a specific hematological cancer. In other embodiments, the HCP set is representative of between one and 18, between one and 16, between one and 14 features, and between one and 12 features of a specific hematological cancer.

In one embodiment of the present invention, genes for inclusion in the HCP sets are selected from the genes set forth in Table 1. Representative, non-limiting examples of HCP sets are provided in Tables 2-19 below. Additional HCP sets representing other hematological cancers, types or sub-types of hematological cancers, or one or more features thereof, can be readily formed by the skilled worker having reference to the genes set forth in Table 1.

The present invention contemplates that these HCP sets can be used as the basis for forming expanded HCP sets that include additional genes to those listed for each set in the Tables below as well as reduced HCP sets from which some, or most, of the genes have been removed. One skilled in the art will also appreciate that combinations of the genes listed in Tables 2-19 below can be used to form additional HCP sets, the genes being selected based on the hematological cancer and features thereof to be investigated. Thus, HCP sets can be formed by combining one or more genes selected from one of the HCP sets provided in Tables 2-19 with one or more genes selected from at least one of the other HCP sets provided in Tables 2-19. All such sets are considered to be within the scope of the invention.

TABLE 2
An HCP set specific for lymphoma, according to one embodiment of the
invention:

| Accession | Symbol |
|---|---|
| AA594161.1 | MYH11 |
| AA766908.1 | MME |
| AB014540.1 | SWAP70 |
| AF196185.1 | PARD3 |
| AI597616.1 | MRPL33 |
| AI634809.1 | ARID5B |
| AI672553.1 | AKAP12 |
| AI809213.1 | RGS13 |
| AK022231.1 | STAT1 |
| AK022293.1 | CTSD |
| AK055652.1 | C3orf6 |
| AL080130.1 | FLJ14001 |
| AL833316.1 | MIR |
| AW271958.1 | TYMS |
| AW291384.1 | STS-1 |
| BE897089.1 | ZNF91 |
| BG993697.1 | SMYD3 |
| BI769730.1 | HLA-DRB1 |
| BQ632574.1 | RASA1 |
| BX504817.1 | SFRS7 |
| H53164.1 | IRF8 |
| H57732.1 | TGFBR2 |
| L29376.1 | 3.8-1 |
| M80899.1 | MGC5395 |
| NM_000038.2 | APC |
| NM_000043.3 | FAS |
| NM_000061.1 | BTK |
| NM_000075.2 | CDK4 |
| NM_000109.2 | DMD |
| NM_000189.4 | HK2 |
| NM_000237.1 | LPL |
| NM_000269.2 | NME1 |
| NM_000271.1 | NPC1 |
| NM_000295.3 | SERPINA1 |
| NM_000313.1 | PROS1 |
| NM_000397.2 | CYBB |
| NM_000405.3 | GM2A |
| NM_000424.2 | KRT5 |
| NM_000483.3 | APOC2 |
| NM_000576.2 | IL1B |
| NM_000579.1 | CCR5 |
| NM_000584.2 | IL8 |
| NM_000585.2 | IL15 |
| NM_000600.1 | IL6 |
| NM_000626.1 | CD79B |
| NM_000627.2 | LTBP1 |
| NM_000633.1 | BCL2 |
| NM_000655.2 | SELL |
| NM_000713.1 | BLVRB |
| NM_000785.2 | CYP27B1 |
| NM_000788.1 | DCK |
| NM_000853.1 | GSTT1 |
| NM_000873.2 | ICAM2 |
| NM_000877.2 | IL1R1 |
| NM_000917.1 | P4HA1 |
| NM_000927.3 | ABCB1 |
| NM_001067.2 | TOP2A |
| NM_001068.2 | TOP2B |
| NM_001110.1 | ADAM10 |
| NM_001154.2 | ANXA5 |
| NM_001166.3 | BIRC2 |
| NM_001196.2 | BID |
| NM_001197.3 | BIK |
| NM_001230.3 | CASP10 |
| NM_001238.1 | CCNE1 |
| NM_001242.3 | TNFRSF7 |
| NM_001254.2 | CDC6 |
| NM_001350.2 | DAXX |
| NM_001353.4 | AKR1C1 |
| NM_001425.1 | EMP3 |
| NM_001552.1 | IGFBP4 |
| NM_001558.2 | IL10RA |
| NM_001618.2 | PARP1 |
| NM_001621.2 | AHR |
| NM_001647.1 | APOD |
| NM_001663.2 | ARF6 |
| NM_001706.2 | BCL6 |
| NM_001760.2 | CCND3 |
| NM_001762.2 | CCT6A |
| NM_001770.3 | CD19 |
| NM_001771.1 | CD22 |
| NM_001779.1 | CD58 |
| NM_001781.1 | CD69 |
| NM_001782.1 | CD72 |
| NM_001826.1 | CKS1B |
| NM_001827.1 | CKS2 |
| NM_001831.2 | CLU |
| NM_001852.3 | COL9A2 |
| NM_001853.2 | COL9A3 |
| NM_001877.2 | CR2 |
| NM_001888.1 | CRYM |
| NM_001894.4 | CSNK1E |
| NM_001951.2 | E2F5 |
| NM_001967.2 | EIF4A2 |
| NM_002002.3 | FCER2 |
| NM_002051.2 | GATA3 |
| NM_002095.3 | GTF2E2 |
| NM_002101.3 | GYPC |
| NM_002105.1 | H2AFX |
| NM_002120.2 | HLA-DOB |
| NM_002122.2 | HLA-DQA1 |
| NM_002155.3 | HSPA6 |
| NM_002189.2 | IL15RA |
| NM_002211.2 | ITGB1 |
| NM_002217.1 | ITIH3 |
| NM_002250.2 | KCNN4 |
| NM_002305.2 | LGALS1 |
| NM_002306.1 | LGALS3 |
| NM_002350.1 | LYN |
| NM_002358.2 | MAD2L1 |
| NM_002426.1 | MMP12 |
| NM_002467.2 | MYC |
| NM_002526.1 | NT5E |
| NM_002567.2 | PBP |
| NM_002646.2 | PIK3C2B |
| NM_002661.1 | PLCG2 |
| NM_002692.2 | POLE2 |
| NM_002738.5 | PRKCB1 |
| NM_002752.3 | MAPK9 |
| NM_002832.2 | PTPN7 |
| NM_002838.2 | PTPRC |
| NM_002891.3 | RASGRF1 |
| NM_002892.2 | ARID4A |
| NM_002943.2 | RORA |
| NM_002966.1 | S100A10 |
| NM_002967.2 | SAFB |
| NM_003037.1 | SLAMF1 |
| NM_003088.2 | FSCN1 |
| NM_003177.3 | SYK |
| NM_003217.1 | TEGT |
| NM_003226.2 | TFF3 |
| NM_003243.1 | TGFBR3 |
| NM_003254.1 | TIMP1 |
| NM_003290.1 | TPM4 |
| NM_003299.1 | TRA1 |
| NM_003332.2 | TYROBP |
| NM_003362.2 | UNG |
| NM_003451.1 | ZNF177 |
| NM_003648.2 | DGKD |
| NM_003656.3 | CAMK1 |
| NM_003810.2 | TNFSF10 |
| NM_003875.1 | GMPS |
| NM_003915.2 | RBM12 |
| NM_003959.1 | HIP1R |
| NM_004049.2 | BCL2A1 |
| NM_004073.2 | PLK3 |
| NM_004091.2 | E2F2 |
| NM_004117.2 | FKBP5 |
| NM_004125.2 | GNG10 |
| NM_004126.2 | GNG11 |
| NM_004184.3 | WARS |
| NM_004271.1 | LY86 |
| NM_004289.5 | NFE2L3 |
| NM_004322.2 | BAD |
| NM_004354.1 | CCNG2 |
| NM_004356.2 | CD81 |
| NM_004358.3 | CDC25B |
| NM_004422.2 | DVL2 |
| NM_004445.1 | EPHB6 |
| NM_004460.2 | FAP |
| NM_004510.2 | SP110 |
| NM_004529.1 | MLLT3 |
| NM_004556.1 | NFKBIE |
| NM_004577.3 | PSPH |
| NM_004619.2 | TRAF5 |
| NM_004635.3 | MAPKAPK3 |
| NM_004737.2 | LARGE |
| NM_004844.1 | SH3BP5 |
| NM_004851.1 | NAPSA |
| NM_004951.2 | EBI2 |
| NM_005013.1 | NUCB2 |
| NM_005077.3 | TLE1 |
| NM_005127.2 | CLECSF2 |
| NM_005211.2 | CSF1R |
| NM_005213.2 | CSTA |
| NM_005214.2 | CTLA4 |
| NM_005271.1 | GLUD1 |
| NM_005292.2 | GPR18 |
| NM_005347.2 | HSPA5 |
| NM_005348.2 | HSPCA |
| NM_005415.3 | SLC20A1 |
| NM_005449.3 | TOSO |
| NM_005461.3 | MAFB |
| NM_005531.1 | IFI16 |
| NM_005582.1 | LY64 |
| NM_005601.3 | NKG7 |
| NM_005606.3 | LGMN |
| NM_005610.1 | RBBP4 |
| NM_005623.2 | CCL8 |
| NM_005638.3 | SYBL1 |
| NM_005652.2 | TERF2 |
| NM_005658.2 | TRAF1 |
| NM_005856.1 | RAMP3 |
| NM_005935.1 | MLLT2 |
| NM_005950.1 | MT1G |
| NM_005951.1 | MT1H |
| NM_006006.3 | ZBTB16 |
| NM_006014.2 | DXS9879E |
| NM_006115.3 | PRAME |
| NM_006162.3 | NFATC1 |
| NM_006164.2 | NFE2L2 |
| NM_006195.2 | PBX3 |
| NM_006218.1 | PIK3CA |
| NM_006254.3 | PRKCD |
| NM_006257.2 | PRKCQ |
| NM_006274.2 | CCL19 |
| NM_006317.3 | BASP1 |
| NM_006332.3 | IFI30 |
| NM_006475.1 | POSTN |
| NM_006498.1 | LGALS2 |
| NM_006558.1 | KHDRBS3 |
| NM_006620.2 | HBS1L |
| NM_006644.2 | HSPH1 |
| NM_006734.2 | HIVEP2 |
| NM_006761.3 | YWHAE |
| NM_006763.2 | BTG2 |
| NM_006841.3 | SLC38A3 |
| NM_006889.2 | CD86 |
| NM_006892.3 | DNMT3B |
| NM_006986.2 | MAGED1 |
| NM_007311.2 | BZRP |
| NM_007360.1 | KLRK1 |
| NM_007361.2 | NID2 |
| NM_012203.1 | GRHPR |
| NM_012448.3 | STAT5B |
| NM_012452.2 | TNFRSF13B |
| NM_013230.1 | CD24 |
| NM_014246.1 | CELSR1 |
| NM_014257.3 | CD209L |
| NM_014338.3 | PISD |
| NM_014456.3 | PDCD4 |
| NM_014686.2 | KIAA0355 |
| NM_014762.1 | DHCR24 |
| NM_015187.1 | KIAA0746 |
| NM_015641.2 | TES |
| NM_015866.2 | PRDM2 |
| NM_016076.2 | PNAS-4 |
| NM_016081.2 | KIAA0992 |
| NM_016187.1 | BIN2 |
| NM_016336.2 | UBE2J1 |
| NM_016628.2 | WAC |
| NM_017530.1 | LOC55565 |
| NM_017784.3 | OSBPL10 |
| NM_017935.2 | BANK1 |
| NM_017955.2 | CDCA4 |
| NM_018442.1 | IQWD1 |
| NM_018842.3 | BAIAP2L1 |
| NM_020151.2 | STARD7 |
| NM_020199.1 | C5orf15 |
| NM_020529.1 | NFKBIA |
| NM_020992.2 | PDLIM1 |
| NM_021813.1 | BACH2 |
| NM_021822.1 | APOBEC3G |
| NM_021950.2 | MS4A1 |
| NM_021966.1 | TCL1A |
| NM_022552.3 | DNMT3A |
| NM_023037.1 | 13CDNA73 |
| NM_024708.2 | ASB7 |
| NM_025113.1 | C13orf18 |
| NM_025216.2 | WNT10A |
| NM_030666.2 | SERPINB1 |
| NM_030764.2 | SPAP1 |
| NM_030775.2 | WNT5B |
| NM_031305.1 | ARHGAP24 |
| NM_031942.3 | CDCA7 |
| NM_032991.1 | CASP3 |
| NM_033208.2 | TIGD7 |
| NM_033274.1 | ADAM19 |
| NM_057735.1 | CCNE2 |
| NM_078467.1 | CDKN1A |
| NM_079421.2 | CDKN2D |
| NM_080593.1 | HIST1H2BK |
| NM_080881.1 | DBN1 |
| NM_138340.3 | ABHD3 |
| NM_138379.1 | TIMD4 |
| NM_139207.1 | NAP1L1 |
| NM_139276.2 | STAT3 |
| NM_144646.2 | IGJ |
| NM_145804.1 | ABTB2 |
| NM_152785.2 | GCET2 |
| NM_172373.2 | ELF1 |
| NM_173624.1 | FLJ40504 |
| NM_175870.3 | LOC90925 |
| NM_177968.1 | PPM1B |
| NM_181443.1 | BTBD3 |
| NM_181838.1 | UBE2D2 |
| NM_182746.1 | MCM4 |
| NM_182776.1 | MCM7 |
| NM_198833.1 | SERPINB8 |
| NM_201593.1 | CACNB2 |
| R99527.1 | MGC39372 |
| T66903.1 | LOC54103 |
| U37028.1 | ITGAD |
| U90916.1 | SORL1 |
| W56129.1 | SFRS9 |
| X58529.1 | IGHM |

TABLE 3
An HCP set specific for leukemia, according to one embodiment of the
invention

| GenBank ™ | | |
|---|---|---|
| Accession No. | Name | Symbol |
| W04885.1 | Myosin, light polypeptide 4, alkali; atrial, embryonic | MYL4 |
| NM_013230.1 | CD24 antigen (small cell lung carcinoma cluster 4 antigen) | CD24 |
| NM_173624.1 | Hypothetical protein FLJ40504 | FLJ40504 |
| NM_002276.3 | Keratin 19 | KRT19 |
| NM_002102.2 | Glycophorin E | GYPE |
| BC058855.1 | Vascular endothelial growth factor | VEGF |
| NM_001353.4 | Aldo-keto reductase family 1, member C2 (dihydrodiol | AKR1C1 |
| | dehydrogenase 2; bile acid binding protein; 3-alpha | |
| | hydroxysteroid dehydrogenase, type III) | |
| NM_002358.2 | MAD2 mitotic arrest deficient-like 1 (yeast) | MAD2L1 |
| NM_018136.2 | Asp (abnormal spindle)-like, microcephaly associated | ASPM |
| | (Drosophila) | |
| NM_005192.2 | Cyclin-dependent kinase inhibitor 3 (CDK2-associated dual | CDKN3 |
| | specificity phosphatase) | |
| NM_001168.1 | Baculoviral IAP repeat-containing 5 (survivin) | BIRC5 |
| NM_006845.2 | Kinesin family member 2C | KIF2C |
| NM_031966.2 | Cyclin B1 | CCNB1 |
| NM_001255.1 | CDC20 cell division cycle 20 homolog (S. cerevisiae) | CDC20 |
| NM_001237.2 | Cyclin A2 | CCNA2 |
| NM_001067.2 | Topoisomerase (DNA) II alpha 170 kDa | TOP2A |
| NM_003234.1 | Transferrin receptor (p90, CD71) | TFRC |
| NM_006101.1 | Kinetochore associated 2 | KNTC2 |
| NM_003038.2 | Solute carrier family 1 (glutamate/neutral amino acid | SLC1A4 |
| | transporter), member 4 | |
| NM_018842.3 | BAI1-associated protein 2-like 1 | BAIAP2L1 |
| NM_006115.3 | Preferentially expressed antigen in melanoma | PRAME |
| NM_031942.3 | Cell division cycle associated 7 | CDCA7 |
| NM_031423.2 | Cell division cycle associated 1 | CDCA1 |
| NM_001786.2 | Cell division cycle 2, G1 to S and G2 to M | CDC2 |
| NM_001254.2 | CDC6 cell division cycle 6 homolog (S. cerevisiae) | CDC6 |
| NM_002692.2 | Polymerase (DNA directed), epsilon 2 (p59 subunit) | POLE2 |
| NM_005563.3 | Stathmin 1/oncoprotein 18 | STMN1 |
| NM_001274.2 | CHK1 checkpoint homolog (S. pombe) | CHEK1 |
| NM_004577.3 | Phosphoserine phosphatase | PSPH |
| NM_005951.1 | Metallothionein 1H | MT1H |
| NM_005950.1 | Metallothionein 1G | MT1G |
| NM_022438.1 | Mal, T-cell differentiation protein | MAL |
| NM_002051.2 | GATA binding protein 3 | GATA3 |
| NM_006748.1 | Src-like-adaptor | SLA |
| NM_005077.3 | Transducin-like enhancer of split 1 (E(sp1) homolog, | TLE1 |
| | Drosophila) | |
| BE927772.1 | Splicing factor, arginine/serine-rich 3 | SFRS3 |
| NM_004615.2 | Transmembrane 4 superfamily member 2 | TM4SF2 |
| NM_000536.1 | Recombination activating gene 2 | RAG2 |
| NM_004088.2 | Deoxynucleotidyltransferase, terminal | DNTT |
| NM_000700.1 | Annexin A1 | ANXA1 |
| NM_201632.1 | Transcription factor 7 (T-cell specific, HMG-box) | TCF7 |
| NM_000732.2 | CD3D antigen, delta polypeptide (TiT3 complex) | CD3D |
| NM_004221.3 | Interleukin 32 | NK4 |
| NM_005601.3 | Natural killer cell group 7 sequence | NKG7 |
| NM_002838.2 | Protein tyrosine phosphatase, receptor type, C | PTPRC |
| NM_003467.1 | Chemokine (C—X—C motif) receptor 4 | CXCR4 |
| NM_001767.2 | CD2 antigen (p50), sheep red blood cell receptor | CD2 |
| NM_005356.2 | Lymphocyte-specific protein tyrosine kinase | LCK |
| NM_006734.2 | Human immunodeficiency virus type I enhancer binding protein 2 | HIVEP2 |
| NM_002412.2 | O-6-methylguanine-DNA methyltransferase | MGMT |
| NM_001079.3 | Zeta-chain (TCR) associated protein kinase 70 kDa | ZAP70 |
| NM_005608.1 | Protein tyrosine phosphatase, receptor type, C-associated | PTPRCAP |
| | protein | |
| NM_002831.3 | Protein tyrosine phosphatase, non-receptor type 6 | PTPN6 |
| T28925.1 | Integrin, alpha L (antigen CD11A (p180), lymphocyte function- | ITGAL |
| | associated antigen 1; alpha polypeptide) | |
| NM_005531.1 | Interferon, gamma-inducible protein 16 | IFI16 |
| NM_012151.2 | Coagulation factor VIII-associated (intronic transcript) 1 | F8A1 |
| NM_000109.2 | Dystrophin (muscular dystrophy, Duchenne and Becker types) | DMD |
| AI672553.1 | A kinase (PRKA) anchor protein (gravin) 12 | AKAP12 |
| BU929213.1 | Sestrin 3 | SESN3 |
| AL049313.1 | Chloride intracellular channel 5 | CLIC5 |
| NM_000856.1 | Guanylate cyclase 1, soluble, alpha 3 | GUCY1A3 |
| NM_005965.3 | Myosin, light polypeptide kinase | MYLK |
| NM_000237.1 | Lipoprotein lipase | LPL |
| AI307786.1 | Transcription factor 4 | TCF4 |
| NM_000089.3 | Collagen, type I, alpha 2 | COL1A2 |
| NM_001839.2 | Calponin 3, acidic | CNN3 |
| NM_006209.2 | Ectonucleotide pyrophosphatase/phosphodiesterase 2 | ENPP2 |
| | (autotaxin) | |
| NM_000062.1 | Serine (or cysteine) proteinase inhibitor, clade G (C1 inhibitor), | SERPING1 |
| | member 1, (angioedema, hereditary) | |
| NM_002658.1 | Plasminogen activator, urokinase | PLAU |
| NM_212474.1 | Fibronectin 1 | FN1 |
| NM_001901.1 | Connective tissue growth factor | CTGF |
| NM_003302.1 | Thyroid hormone receptor interactor 6 | TRIP6 |
| NM_000305.1 | Paraoxonase 2 | PON2 |
| AK055652.1 | Chromosome 3 open reading frame 6 | C3orf6 |
| NM_003821.4 | Receptor-interacting serine-threonine kinase 2 | RIPK2 |
| BC050602.1 | histone 1, H2ac | HIST1H2AC |
| NM_002870.2 | RAB13, member RAS oncogene family | RAB13 |
| NM_007311.2 | Benzodiazapine receptor (peripheral) | BZRP |
| NM_030666.2 | Serine (or cysteine) proteinase inhibitor, clade B (ovalbumin), | SERPINB1 |
| | member 1 | |
| NM_003254.1 | Tissue inhibitor of metalloproteinase 1 (erythroid potentiating | TIMP1 |
| | activity, collagenase inhibitor) | |
| NM_198232.1 | Ribonuclease, RNase A family, 1 (pancreatic) | RNASE1 |
| NM_015149.2 | Ral guanine nucleotide dissociation stimulator-like 1 | RGL1 |
| NM_025113.1 | Chromosome 13 open reading frame 18 | C13orf18 |
| NM_021966.1 | T-cell leukemia/lymphoma 1A | TCL1A |
| NM_002002.3 | Fc fragment of IgE, low affinity II, receptor for (CD23A) | FCER2 |
| NM_002661.1 | Phospholipase C, gamma 2 (phosphatidylinositol-specific) | PLCG2 |
| AK022231.1 | Signal transducer and activator of transcription 1, 91 kDa | STAT1 |
| NM_006317.3 | Brain abundant, membrane attached signal protein 1 | BASP1 |
| NM_006120.2 | Major histocompatibility complex, class II, DM alpha | HLA-DMA |
| NM_001774.1 | CD37 antigen | CD37 |
| NM_144646.2 | Immunoglobulin J polypeptide, linker protein for | IGJ |
| | immunoglobulin alpha and mu polypeptides | |
| NM_003121.1 | Spi-B transcription factor (Spi-1/PU.1 related) | SPIB |
| NM_021950.2 | Membrane-spanning 4-domains, subfamily A, member 1 | MS4A1 |
| NM_001877.2 | Complement component (3d/Epstein Barr virus) receptor 2 | CR2 |
| NM_005658.2 | TNF receptor-associated factor 1 | TRAF1 |
| NM_004513.3 | Interleukin 16 (lymphocyte chemoattractant factor) | IL16 |
| NM_002341.1 | Lymphotoxin beta (TNF superfamily, member 3) | LTB |
| AI523104.1 | Leukocyte immunoglobulin-like receptor, subfamily B (with | LILRB1 |
| | TM and ITIM domains), member 1 | |
| NM_013416.2 | Neutrophil cytosolic factor 4, 40 kDa | NCF4 |
| NM_004355.1 | CD74 antigen (invariant polypeptide of major | CD74 |
| | histocompatibility complex, class II antigen-associated) | |
| NM_005213.2 | Cystatin A (stefin A) | CSTA |
| BC042366.1 | protocadherin 9 | PCDH9 |
| NM_001337.2 | Chemokine (C—X3—C motif) receptor 1 | CX3CR1 |
| NM_002619.1 | Platelet factor 4 (chemokine (C—X—C motif) ligand 4) | PF4 |
| NM_002704.2 | Pro-platelet basic protein (chemokine (C—X—C motif) ligand 7) | PPBP |
| NM_015570.1 | Autism susceptibility candidate 2 | AUTS2 |
| U90916.1 | Sortilin-related receptor, L(DLR class) A repeats-containing | SORL1 |
| NM_004049.2 | BCL2-related protein A1 | BCL2A1 |
| NM_015474.2 | SAM domain and HD domain 1 | SAMHD1 |
| NM_004951.2 | Epstein-Barr virus induced gene 2 (lymphocyte-specific G | EBI2 |
| | protein-coupled receptor) | |
| NM_003037.1 | Signaling lymphocytic activation molecule family member 1 | SLAMF1 |
| NM_156038.2 | Colony stimulating factor 3 receptor (granulocyte) | CSF3R |
| NM_005248.1 | Gardner-Rasheed feline sarcoma viral (v-fgr) oncogene | FGR |
| | homolog | |
| NM_016184.2 | C-type lectin domain family 4, member A | CLECSF6 |
| NM_001776.3 | Ectonucleoside triphosphate diphosphohydrolase 1 | ENTPD1 |
| NM_004851.1 | Napsin A aspartic peptidase | NAPSA |
| NM_000484.2 | Amyloid beta (A4) precursor protein (protease nexin-II, | APP |
| | Alzheimer disease) | |
| NM_000626.1 | CD79B antigen (immunoglobulin-associated beta) | CD79B |
| NM_001242.3 | Tumor necrosis factor receptor superfamily, member 7 | TNFRSF7 |
| NM_006820.1 | Interferon-induced protein 44-like | IFI44L |
| NM_021822.1 | Apolipoprotein B mRNA editing enzyme, catalytic polypeptide- | APOBEC3G |
| | like 3F | |
| NM_000061.1 | Bruton agammaglobulinemia tyrosine kinase | BTK |
| NM_002985.2 | Chemokine (C-C motif) ligand 5 | CCL5 |
| NM_001558.2 | Interleukin 10 receptor, alpha | IL10RA |
| NM_006254.3 | Protein kinase C, delta | PRKCD |
| NM_002306.1 | Lectin, galactoside-binding, soluble, 3 (galectin 3) | LGALS3 |
| NM_000610.3 | CD44 antigen (homing function and Indian blood group system) | CD44 |
| NM_004271.1 | Lymphocyte antigen 86 | LY86 |
| AB014540.1 | SWAP-70 protein | SWAP70 |
| NM_001165.3 | Baculoviral IAP repeat-containing 3 | BIRC3 |
| NM_006889.2 | CD86 antigen (CD28 antigen ligand 2, B7-2 antigen) | CD86 |
| NM_002110.2 | Hemopoietic cell kinase | HCK |
| NM_003810.2 | Tumor necrosis factor (ligand) superfamily, member 10 | TNFSF10 |
| T66903.1 | Hypothetical protein LOC54103 | LOC54103 |
| NM_005335.3 | Hematopoietic cell-specific Lyn substrate 1 | HCLS1 |
| NM_005767.3 | Purinergic receptor P2Y, G-protein coupled, 5 | P2RY5 |
| NM_007360.1 | Killer cell lectin-like receptor subfamily K, member 1 | KLRK1 |
| NM_006498.1 | Lectin, galactoside-binding, soluble, 2 (galectin 2) | LGALS2 |
| NM_000118.1 | Endoglin (Osler-Rendu-Weber syndrome 1) | ENG |
| AL050391.1 | Caspase 4, apoptosis-related cysteine protease | CASP4 |
| NM_005211.2 | Colony stimulating factor 1 receptor, formerly McDonough | CSF1R |
| | feline sarcoma viral (v-fms) oncogene homolog | |
| NM_030764.2 | SH2 domain containing phosphatase anchor protein 1 | SPAP1 |
| NM_001001547.1 | CD36 antigen (collagen type I receptor, thrombospondin | CD36 |
| | receptor) | |
| NM_005621.1 | S100 calcium binding protein A12 (calgranulin C) | S100A12 |
| NM_000295.3 | Serine (or cysteine) proteinase inhibitor, clade A (alpha-1 | SERPINA1 |
| | antiproteinase, antitrypsin), member 1 | |
| NM_005084.2 | Phospholipase A2, group VII (platelet-activating factor | PLA2G7 |
| | acetylhydrolase, plasma) | |
| NM_021038.3 | Muscleblind-like (Drosophila) | MBNL1 |
| NM_001553.1 | Insulin-like growth factor binding protein 7 | IGFBP7 |
| NM_006332.3 | Interferon, gamma-inducible protein 30 | IFI30 |
| NM_002305.2 | Lectin, galactoside-binding, soluble, 1 (galectin 1) | LGALS1 |
| NM_002961.2 | S100 calcium binding protein A4 (calcium protein, calvasculin, | S100A4 |
| | metastasin, murine placental homolog) | |
| NM_006472.1 | Thioredoxin interacting protein | TXNIP |
| NM_014624.2 | S100 calcium binding protein A6 (calcyclin) | S100A6 |
| NM_003332.2 | TYRO protein tyrosine kinase binding protein | TYROBP |
| NM_033554.2 | Major histocompatibility complex, class II, DP alpha 1 | HLA-DPA1 |
| NM_002121.4 | Major histocompatibility complex, class II, DP beta 1 | HLA-DPB1 |
| NM_019111.2 | Major histocompatibility complex, class II, DR alpha | HLA-DRA |

TABLE 4
An HCP set specific for ALCL, according to one embodiment of the
invention

| Feature | Accession | Symbol | Name | Notes | Level |
|---|---|---|---|---|---|
| ALCL | NM_001831 | CLU | Clusterin | Diagnostic | Up in |
| Signature | | | | Marker | ALCL |
| ALK+ | NM_000295 | SERPINA1 | serine (or cysteine) | Genes up | Up in |
| vs. ALK− | | | proteinase inhibitor, clade A | regulated in | ALK+ |
| (ALCL) | | | (aHCPha-1 antiproteinase, | ALK+ ALCL | ALCL vs |
| | | | antitrypsin), member 1 | | ALK− |
| ALK+ | NM_022743 | SMYD3 | SET and MYND domain | Genes up | Up in |
| vs. ALK− | | | containing 3 | regulated in | ALK+ |
| (ALCL) | | | | ALK+ ALCL | ALCL vs |
| | | | | | ALK− |
| ALK+ | NM_003217 | TEGT | testis enhanced gene | Genes up | Up in |
| vs. ALK− | | | transcript (BAX inhibitor 1) | regulated in | ALK+ |
| (ALCL) | | | | ALK+ ALCL | ALCL vs |
| | | | | | ALK− |
| ALK+ | NM_003352 | UBL1 | ubiquitin-like 1 (sentrin) | Genes up | Up in |
| vs. ALK− | | | | regulated in | ALK+ |
| (ALCL) | | | | ALK+ ALCL | ALCL vs |
| | | | | | ALK− |
| ALK+ | NM_004762 | PSCD1 | pleckstrin homology, Sec7 | Genes up | Up in |
| vs. ALK− | | | and coiled-coil domains | regulated in | ALK+ |
| (ALCL) | | | 1(cytohesin 1) | ALK+ ALCL | ALCL vs |
| | | | | | ALK− |
| ALK+ | NM_002444 | MSN | moesin | Genes up | Up in |
| vs. ALK− | | | | regulated in | ALK+ |
| (ALCL) | | | | ALK+ ALCL | ALCL vs |
| | | | | | ALK− |
| ALK+ | NM_006325 | RAN | RAN, member RAS | Genes up | Up in |
| vs. ALK− | | | oncogene family | regulated in | ALK+ |
| (ALCL) | | | | ALK+ ALCL | ALCL vs |
| | | | | | ALK− |
| ALK+ | NM_006579 | EBP | emopamil binding protein | Genes up | Up in |
| vs. ALK− | | | (sterol isomerase) | regulated in | ALK+ |
| (ALCL) | | | | ALK+ ALCL | ALCL vs |
| | | | | | ALK− |
| ALK+ | NM_032263 | DKFZp434B227 | hypothetical protein | Genes up | Up in |
| vs. ALK− | | | DKFZp434B227 | regulated in | ALK+ |
| (ALCL) | | | | ALK+ ALCL | ALCL vs |
| | | | | | ALK− |
| ALK+ | NM_014670 | BZW1 | basic leucine zipper and W2 | Genes up | Up in |
| vs. ALK− | | | domains 1 | regulated in | ALK+ |
| (ALCL) | | | | ALK+ ALCL | ALCL vs |
| | | | | | ALK− |
| ALK+ | NM_000877 | IL1R1 | interleukin 1 receptor, type I | Genes up | Up in |
| vs. ALK− | | | | regulated in | ALK+ |
| (ALCL) | | | | ALK+ ALCL | ALCL vs |
| | | | | | ALK− |
| ALK+ | NM_018209 | ARFGAP1 | ADP-ribosylation factor | Genes up | Up in |
| vs. ALK− | | | GTPase activating protein 1 | regulated in | ALK+ |
| (ALCL) | | | | ALK+ ALCL | ALCL vs |
| | | | | | ALK− |
| ALK+ | NM_001923 | DDB1 | damage-specific DNA | Genes up | Up in |
| vs. ALK− | | | binding protein 1, 127 kDa | regulated in | ALK+ |
| (ALCL) | | | | ALK+ ALCL | ALCL vs |
| | | | | | ALK− |
| ALK+ | NM_001618 | ADPRT | ADP-ribosyltransferase | Genes up | Up in |
| vs. ALK− | | | (NAD+; poly (ADP-ribose) | regulated in | ALK+ |
| (ALCL) | | | polymerase) | ALK+ ALCL | ALCL vs |
| | | | | | ALK− |
| ALK+ | NM_021960 | MCL1 | myeloid cell leukemia | Genes up | Up in |
| vs. ALK− | | | sequence 1 (BCL2-related) | regulated in | ALK+ |
| (ALCL) | | | | ALK+ ALCL | ALCL vs |
| | | | | | ALK− |
| ALK+ | NM_001165 | BIRC3 | baculoviral IAP repeat- | Genes up | Up in |
| vs. ALK− | | | containing 3 | regulated in | ALK+ |
| (ALCL) | | | | ALK+ ALCL | ALCL vs |
| | | | | | ALK− |
| ALK+ | NM_018462 | MDS027 | uncharacterized | Genes up | Up in |
| vs. ALK− | | | hematopoietic | regulated in | ALK+ |
| (ALCL) | | | stem/progenitor cells protein | ALK+ ALCL | ALCL vs |
| | | | MDS027 | | ALK− |
| ALK+ | AB032991 | NDFIP2 | Nedd4 family interacting | Genes up | Up in |
| vs. ALK− | | | protein 2 | regulated in | ALK+ |
| (ALCL) | | | | ALK+ ALCL | ALCL vs |
| | | | | | ALK− |
| ALK+ | NM_000417 | IL2RA | interleukin 2 receptor, | Genes up | Up in |
| vs. ALK− | | | aHCPha | regulated in | ALK+ |
| (ALCL) | | | | ALK+ ALCL | ALCL vs |
| | | | | | ALK− |
| ALK+ | NM_031966 | CCNB1 | cyclin B1 | Genes up | Up in |
| vs. ALK− | | | | regulated in | ALK+ |
| (ALCL) | | | | ALK+ ALCL | ALCL vs |
| | | | | | ALK− |
| ALK+ | NM_030674 | SLC38A1 | solute carrier family 38, | Genes up | Up in |
| vs. ALK− | | | member 1 | regulated in | ALK+ |
| (ALCL) | | | | ALK+ ALCL | ALCL vs |
| | | | | | ALK− |
| ALK+ | NM_022366 | TFB2M | transcription factor B2, | Genes up | Up in |
| vs. ALK− | | | mitochondrial | regulated in | ALK+ |
| (ALCL) | | | | ALK+ ALCL | ALCL vs |
| | | | | | ALK− |
| HL vs. | NM_002228 | JUN | v-jun sarcoma virus 17 | Differentially | Up only in |
| ALCL | | | oncogene homolog (avian) | regulated | HL |
| | | | | between ALCL | |
| | | | | and HL | |
| HL vs. | NM_002961 | S100A4 | S100 calcium binding | Differentially | Up only in |
| ALCL | | | protein A4 (calcium protein, | regulated | HL |
| | | | calvasculin, metastasin, | between ALCL | |
| | | | murine placental homolog) | and HL | |
| HL vs. | NM_005178 | BCL3 | B-cell CLL/lymphoma 3 | Diagnostic | Up only in |
| ALCL | | | | Marker | ALCL |
| HL vs. | NM_176783 | PSME1 | proteasome (prosome, | Differentially | Up only in |
| ALCL | | | macropain) activator subunit | regulated | ALCL |
| | | | 1 (PA28 aHCPha) | between ALCL | |
| | | | | and HL | |
| HL vs. | NM_000485 | APRT | adenine | Differentially | Up in Both |
| ALCL | | | phosphoribosyltransferase | regulated | HL and |
| | | | | between ALCL | ALCL |
| | | | | and HL | |
| HL vs. | NM_001728 | BSG | basigin (OK blood group) | Differentially | Up only in |
| ALCL | | | | regulated | ALCL |
| | | | | between ALCL | |
| | | | | and HL | |
| HL vs. | NM_006763 | BTG2 | BTG family member 2 | Differentially | Up in Both |
| ALCL | | | | regulated | HL and |
| | | | | between ALCL | ALCL |
| | | | | and HL | |
| HL vs. | NM_001743 | CALM2 | calmodulin 2 (phosphorylase | Differentially | Up in Both |
| ALCL | | | kinase, delta) | regulated | HL and |
| | | | | between ALCL | ALCL |
| | | | | and HL | |
| HL vs. | NM_002086 | GRB2 | growth factor receptor-bound | Differentially | Up only in |
| ALCL | | | protein 2 | regulated | HL |
| | | | | between ALCL | |
| | | | | and HL | |
| HL vs. | NM_005340 | HINT1 | histidine triad nucleotide | Differentially | Up only in |
| ALCL | | | binding protein 1 | regulated | ALCL |
| | | | | between ALCL | |
| | | | | and HL | |
| HL vs. | NM_006191 | PA2G4 | proliferation-associated 2G4, | Differentially | Up in Both |
| ALCL | | | 38 kDa | regulated | HL and |
| | | | | between ALCL | ALCL |
| | | | | and HL | |
| HL vs. | NM_030666 | SERPINB1 | seine (or cysteine) | Differentially | Up in Both |
| ALCL | | | proteinase inhibitor, clade B | regulated | HL and |
| | | | (ovalbumin), member 1 | between ALCL | ALCL |
| | | | | and HL | |
| HL vs. | NM_003955 | SOCS3 | suppressor of cytokine | Differentially | Up only in |
| ALCL | | | signaling 3 | regulated | ALCL |
| | | | | between ALCL | |
| | | | | and HL | |
| HL vs. | NM_003254 | TIMP1 | tissue inhibitor of | Genes unique | Disagreement: |
| ALCL | | | metalloproteinase 1 | to | Up in |
| | | | (erythroid potentiating | Reed/Sternberg | HL; Up |
| | | | activity, collagenase | cells; | only in |
| | | | inhibitor) | Differentially | ALCL; Up |
| | | | | regulated | in Fatal; |
| | | | | between ALCL | Down in |
| | | | | and HL; Genes | Cured |
| | | | | with prognostic | |
| | | | | value | |
| HL vs. | NM_033306 | CASP4 | caspase 4, apoptosis-related | Differentially | Up only in |
| ALCL | | | cysteine protease | regulated | ALCL |
| | | | | between ALCL | |
| | | | | and HL | |
| Signature; | NM_003254 | TIMP1 | tissue inhibitor of | Genes unique | Disagreement: |
| HL vs. | | | metalloproteinase 1 | to | Up in |
| ALCL; | | | (erythroid potentiating | Reed/Sternberg | HL; Up |
| Survival | | | activity, collagenase | cells; | only in |
| | | | inhibitor) | Differentially | ALCL; Up |
| | | | | regulated | in Fatal; |
| | | | | between ALCL | Down in |
| | | | | and HL; Genes | Cured |
| | | | | with prognostic | |
| | | | | value | |

TABLE 5
An HCP set specific for CLL/SLL, according to one embodiment of the invention

| Feature | Accession | Symbol | Name | Notes | Level |
|---|---|---|---|---|---|
| CLL/SLL | NM_020944 | GBA2 | glucosidase, beta (bile | | down in |
| vs MCL | | | acid) 2 | | MCL vs |
| | | | | | SLL (−5.1) |
| CLL/SLL | NM_003222 | TFAP2C | transcription factor AP- | | down in |
| | | | 2 gamma (activating | | CLL/SLL |
| | | | enhancer binding | | (−7.2) |
| | | | protein 2 gamma) | | |
| CLL/SLL | NM_019846 | CCL28 | chemokine (C-C motif) | | up in FL |
| vs FL | | | ligand 28 | | vs |
| | | | | | CLL/SLL |
| | | | | | (5.2) |
| CLL/SLL | NM_004827 | ABCG2 | ATP-binding cassette, | | down in |
| | | | sub-family G | | CLL/SLL |
| | | | (WHITE), member 2 | | (−7.2) |
| CLL/SLL | NM_001830 | CLCN4 | chloride channel 4 | | down in |
| vs MCL | | | | | MCL vs |
| | | | | | SLL (−4.9) |
| CLL vs. | NM_000633 | BCL2 | B-cell CLL/lymphoma 2 | CLL signature vs. | High in |
| DLBCL | | | | DLBCL | CLL |
| CLL/SLL | NM_001237 | CCNA2 | cyclin A2 | | Down in |
| | | | | | CLL/SLL |
| | | | | | (−5) |
| CLL vs. | NM_053056 | CCND1 | cyclin D1 | MCL, CLL | High in |
| MCL | | | (BCL1/PRAD1: | Signature&CLL | CLL |
| | | | parathyroid | Vs. MCL | |
| | | | adenomatosis 1) | Distinguisher | |
| MCL vs | NM_003651 | CSDA | cold shock domain | | down in |
| CLL/SLL | | | protein A | | MCL vs |
| | | | | | SLL (−4.4) |
| CLL vs. | NM_002390 | ADAM11 | a disintegrin and | Resting B-cell | High in |
| DLBCL | | | metalloproteinase | signature | CLL |
| | | | domain 11 | | |
| CLL vs. | NM_000698 | ALOX5 | arachidonate 5- | Resting B-cell | High in |
| DLBCL | | | lipoxygenase | signature | CLL |
| CLL vs. | NM_001706 | BCL6 | B-cell CLL/lymphoma | High in G-center | Low in |
| DLBCL | | | 6 (zinc finger protein | type DLBCL | CLL; |
| | | | 51) | | High in |
| | | | | | G-center |
| | | | | | DLBCL |
| CLL vs. | NM_001197 | BIK | BCL2-interacting killer | High in G-center | Low in |
| DLBCL | | | (apoptosis-inducing) | type DLBCL | CLL; |
| | | | | | High in |
| | | | | | G-center |
| | | | | | DLBCL |
| CLL vs. | NM_016187 | BIN2 | bridging integrator 2 | Resting B-cell | High in |
| DLBCL | | | | signature | CLL |
| CLL vs. | NM_014207 | CD5 | CD5 antigen (p56-62) | Hs.58685 | High in |
| DLBCL | | | | | CLL |
| CLL vs. | NM_001775 | CD38 | CD38 antigen (p45) | Hs.174944; | Low in |
| DLBCL | | | | Prognostically | CLL; |
| | | | | significant | High in |
| | | | | | G-center |
| | | | | | DLBCL |
| CLL/SLL | NM_002984 | CCL4 | chemokine (C-C motif) | | down in |
| | | | ligand 4 | | SLL (−8.7) |
| MCL vs | NM_002466 | MYBL2 | v-myb myeloblastosis | | up in |
| CLL/SLL | | | viral oncogene | | MCL vs |
| | | | homolog (avian)-like 2 | | SLL (5.1) |
| CLL/SLL | NM_025113 | C13orf18 | chromosome 13 open | | up in |
| | | | reading frame 18 | | SLL (4.8) |
| FL vs | NM_025113 | C13orf18 | chromosome 13 open | | down in |
| CLL/SLL | | | reading frame 18 | | FL vs |
| | | | | | SLL (−5.3) |
| MCL vs | NM_032873 | KIAA1959 | nm23-phosphorylated | | down in |
| CLL/SLL | | | unknown substrate | | MCL vs |
| | | | | | SLL (−5.3) |
| CLL/SLL | NM_000633 | BCL2 | B-cell CLL/lymphoma 2 | | Up in |
| | | | | | SLL (4.4) |
| MCL vs | NM_000418 | IL4R | interlekin 4 receptor | | down in |
| CLL/SLL | | | | | MCL vs |
| | | | | | SLL (−7.1) |
| CLL/SLL | NM_004630 | SF1 | splicing factor 1 | | down in |
| | | | | | CLL/SLL |
| | | | | | (−7) |
| CLL/SLL | AF509494 | KIAA1407 | KIAA1407 protein | | up in |
| | | | | | SLL (4.1) |
| CLL/SLL | NM_006565 | CTCF | CCCTC-binding factor | | down in |
| | | | (zinc finger protein) | | SLL (−4.1); |
| | | | | | up |
| | | | | | in FL vs |
| | | | | | SLL |
| | | | | | (10.2) |
| CLL/SLL | NM_000483 | APOC2 | apolipoprotein C-II | | up in |
| vs MCL | | | | | MCL vs |
| | | | | | SLL (5.6) |
| CLL/SLL | AB018263 | KIAA0720 | putative NFkB | | Up in |
| vs MCL | | | activating protein | | MCL vs |
| | | | | | SLL (4.3) |
| CLL/SLL | NM_003915 | CPNE1 | copine I | | up in |
| | | | | | SLL |
| | | | | | (7.1); up |
| | | | | | in FL vs |
| | | | | | SLL (−4.5) |
| CLL/SLL | NM_005248 | FGR | Gardner-Rasheed feline | | down in |
| | | | sarcoma viral (v-fgr) | | FL vs |
| | | | oncogene homolog | | SLL (−5.8); |
| | | | | | down in |
| | | | | | MCL vs |
| | | | | | SLL (−4.5) |
| CLL/SLL | NM_003480 | MFAP5 | microfibrillar | | up in |
| vs MCL | | | associated protein 5 | | MCL vs |
| | | | | | CLL/SLL |
| | | | | | (4.2) |
| CLL vs. | NM_005248 | FGR | Gardner-Rasheed feline | Resting B-cell | High in |
| DLBCL | | | sarcoma viral (v-fgr) | signature | CLL |
| | | | oncogene homolog | | |
| MCL vs | NM_023037 | 13CDNA73 | hypothetical protein | | MCL vs |
| CLL/SLL | | | CG003 | | SLL (−7.6) |
| Mutational | NM_002466 | MYBL2 | v-myb myeloblastosis | Ig mutation | Low in |
| Status | | | viral oncogene | indicator: Low in | Mutated |
| (CLL) | | | homolog (avian)-like 2 | mut | |
| CLL vs. | NM_005214 | CTLA4 | cytotoxic T- | CLL signature vs. | High in |
| DLBCL | | | lymphocyte-associated | DLBCL | CLL |
| | | | protein 4 | | |
| CLL vs. | NM_000075 | CDK4 | cyclin-dependent | MCL, CLL | High in |
| MCL | | | kinase 4 | Signature&CLL | CLL |
| | | | | Vs. MCL | |
| | | | | Distinguisher | |
| CLL vs. | NM_004064 | CDKN1B | cyclin-dependent | MCL, CLL | High in |
| MCL | | | kinase inhibitor 1B | Signature&CLL | CLL |
| | | | (p27, Kip1) | Vs. MCL | |
| | | | | Distinguisher | |
| CLL vs. | NM_004454 | ETV5 | ets variant gene 5 (ets- | RT-PCR analysis | High in |
| MCL | | | related molecule) | shows up | CLL |
| | | | | regulation in CLL | |
| CLL vs. | NM_002467 | MYC | v-myc | RT-PCR analysis | High in |
| MCL | | | myelocytomatosis viral | shows up | CLL |
| | | | oncogene homolog | regulation in CLL | |
| | | | (avian) | | |
| Mutational | NM_005100 | AKAP12 | A kinase (PRKA) | Ig mutation | Low in |
| Status | | | anchor protein (gravin) | indicator: Low in | Mutated |
| (CLL) | | | 12 | mut | |
| CLL vs. | NM_032663 | USP30 | ubiquitin specific | CLL signature vs. | High in |
| DLBCL | | | protease 30 | DLBCL | CLL |
| Mutational | NM_001826 | CKS1B | CDC28 protein kinase | Ig mutation | Low in |
| Status | | | regulatory subunit 1B | indicator: Low in | Mutated |
| (CLL) | | | | mut | |
| CLL vs. | NM_006472 | TXNIP | thioredoxin interacting | Resting B-cell | High in |
| DLBCL | | | protein | signature | CLL |
| Mutational | NM_006187 | OAS3 | 2′-5′-oligoadenylate | Ig mutation | Low in |
| Status | | | synthetase 3, 100 kDa | indicator: Low in | Mutated |
| (CLL) | | | | mut | |
| CLL/SLL | NM_002933 | RNASE1 | ribonuclease, RNase A | | down in |
| vs MCL | | | family, 1 (pancreatic) | | MCL vs |
| | | | | | SLL (−5.4) |
| CLL/SLL | NM_006820 | C1orf29 | chromosome 1 open | | down in |
| vs FL | | | reading frame 29 | | FL vs |
| | | | | | MCL (−4.3) |
| CLL/SLL | BC046632 | FLJ21195 | protein related to DAN | | up in FL |
| vs FL | | | and cerberus | | vs SLL |
| | | | | | (4.1) |
| CLL/SLL | NM_024713 | FLJ22557 | hypothetical protein | | up in |
| | | | FLJ22557 | | SLL |
| | | | | | (11.9) |
| CLL/SLL | NM_031305 | ARHGAP24 | Rho GTPase activating | | up in |
| | | | protein 24 | | SLL (4.4) |
| Mutational | NM_017935 | BANK1 | B-cell scaffold protein | Ig mutation | High in |
| Status | | | with ankyrin repeats 1 | indicator: high in | Mutated |
| (CLL) | | | | mut | |
| CLL vs. | XM_034274 | MYBL1 | v-myb myeloblastosis | High in G-center | Low in |
| DLBCL | | | viral oncogene | type DLBCL | CLL; |
| | | | homolog (avian)-like 1 | | High in |
| | | | | | G-center |
| | | | | | DLBCL |
| CLL/SLL | NM_014686 | KIAA0355 | KIAA0355 | | down in |
| vs FL | | | | | FL vs |
| | | | | | SLL (−4.5 ) |
| CLL vs. | NM_058176 | HDAC9 | histone deacetylase 9 | CLL signature vs. | High in |
| DLBCL | | | | DLBCL | CLL |
| CLL vs. | NM_006850 | IL24 | interleukin 24 | Resting B-cell | High in |
| DLBCL | | | | signature | CLL |
| CLL vs. | NM_000417 | IL2RA | interleukin 2 receptor, | CLL signature vs. | High in |
| DLBCL | | | aHCPha | DLBCL | CLL |
| CLL vs. | NM_000418 | IL4R | interleukin 4 receptor | Resting B-cell | High in |
| DLBCL | | | | signature | CLL |
| CLL vs. | NM_005574 | LMO2 | LIM domain only 2 | High in G-center | Low in |
| DLBCL | | | (rhombotin-like 1) | type DLBCL | CLL; |
| | | | | | High in |
| | | | | | G-center |
| | | | | | DLBCL |
| CLL vs. | NM_001760 | CCND3 | cyclin D3 | CLL Vs. MCL | High in |
| MCL | | | | Distinguisher | CLL |
| CLL vs. | NM_007289 | MME | membrane metallo- | High in G-center | Low in |
| DLBCL | | | endopeptidase (neutral | type DLBCL | CLL; |
| | | | endopeptidase, | | High in |
| | | | enkephalinase, | | G-center |
| | | | CALLA, CD10) | | DLBCL |
| CLL vs. | NM_006495 | EVI2B | ecotropic viral | Resting B-cell | High in |
| DLBCL | | | integration site 2B | signature | CLL |
| CLL vs. | NM_002738 | PRKCB1 | protein kinase C, beta 1 | Resting B-cell | High in |
| DLBCL | | | | signature | CLL |
| CLL vs. | NM_002835 | PTPN12 | protein tyrosine | CLL signature vs. | High in |
| DLBCL | | | phosphatase, non- | DLBCL | CLL |
| | | | receptor type 12 | | |
| CLL vs. | NM_002927 | RGS13 | regulator of G-protein | High in G-center | Low in |
| DLBCL | | | signalling 13 | type DLBCL | CLL; |
| | | | | | High in |
| | | | | | G-center |
| | | | | | DLBCL |
| CLL vs. | NM_003005 | SEHCP | selectin P (granule | CLL signature vs. | High in |
| DLBCL | | | membrane protein | DLBCL | CLL |
| | | | 140 kDa, antigen | | |
| | | | CD62) | | |
| CLL vs. | NM_005449 | TOSO | regulator of Fas- | Resting B-cell | High in |
| DLBCL | | | induced apoptosis | signature | CLL |
| CLL vs. | NM_133378 | TTN | titin | CLL signature vs. | High in |
| DLBCL | | | | DLBCL | CLL |
| CLL vs. | XM_042066 | MAP3K1 | mitogen-activated | Resting B-cell | High in |
| DLBCL | | | protein kinase kinase | signature | CLL |
| | | | kinase 1 | | |

TABLE 6
An HCP set specific for DLBCL, according to one embodiment of the
invention

| Feature | Accession | Symbol | Name | Notes | Level |
|---|---|---|---|---|---|
| CLL vs. | XM_042066 | MAP3K1 | mitogen-activated | Resting B-cell | High in CLL |
| DLBCL | | | protein kinase kinase | signature | |
| | | | kinase 1 | | |
| DLBCL Vs. | NM_006472 | TXNIP | thioredoxin interacting | Genes | Down in |
| FL | | | protein | specific to | DLBCL, Up in |
| | | | | either | FL |
| | | | | DLBCL or | |
| | | | | FL | |
| CLL vs. | NM_003005 | SEHCP | selectin P (granule | CLL | High in CLL |
| DLBCL | | | membrane protein | signature vs. | |
| | | | 140 kDa, antigen CD62) | DLBCL | |
| CLL vs. | NM_002927 | RGS13 | regulator of G-protein | High in G- | Low in CLL; |
| DLBCL | | | signalling 13 | center type | High in G- |
| | | | | DLBCL | center DLBCL |
| CLL vs. | NM_002835 | PTPN12 | protein tyrosine | CLL | High in CLL |
| DLBCL | | | phosphatase, non- | signature vs. | |
| | | | receptor type 12 | DLBCL | |
| CLL vs. | NM_002738 | PRKCB1 | protein kinase C, beta 1 | Resting B-cell | High in CLL |
| DLBCL | | | | signature | |
| CLL vs. | NM_133378 | TTN | titin | CLL | High in CLL |
| DLBCL | | | | signature vs. | |
| | | | | DLBCL | |
| CLL vs. | NM_007289 | MME | membrane metallo- | High in G- | Low in CLL; |
| DLBCL | | | endopeptidase (neutral | center type | High in G- |
| | | | endopeptidase, | DLBCL | center DLBCL |
| | | | enkephalinase, CALLA, | | |
| | | | CD10) | | |
| CLL vs. | NM_006472 | TXNIP | thioredoxin interacting | Resting B-cell | High in CLL |
| DLBCL | | | protein | signature | |
| CLL vs. | NM_005574 | LMO2 | LIM domain only 2 | High in G- | Low in CLL; |
| DLBCL | | | (rhombotin-like 1) | center type | High in G- |
| | | | | DLBCL | center DLBCL |
| CLL vs. | NM_000418 | IL4R | interleukin 4 receptor | Resting B-cell | High in CLL |
| DLBCL | | | | signature | |
| CLL vs. | NM_000417 | IL2RA | interleukin 2 receptor, | CLL | High in CLL |
| DLBCL | | | aHCPha | signature vs. | |
| | | | | DLBCL | |
| CLL vs. | NM_006850 | IL24 | interleukin 24 | Resting B-cell | High in CLL |
| DLBCL | | | | signature | |
| CLL vs. | NM_058176 | HDAC9 | histone deacetylase 9 | CLL | High in CLL |
| DLBCL | | | | signature vs. | |
| | | | | DLBCL | |
| CLL vs. | NM_005248 | FGR | Gardner-Rasheed feline | Resting B-cell | High in CLL |
| DLBCL | | | sarcoma viral (v-fgr) | signature | |
| | | | oncogene homolog | | |
| CLL vs. | XM_034274 | MYBL1 | v-myb myeloblastosis | High in G- | Low in CLL; |
| DLBCL | | | viral oncogene homolog | center type | High in G- |
| | | | (avian)-like 1 | DLBCL | center DLBCL |
| DLBCL Vs. | NM_006152 | LRMP | lymphoid-restricted | Shipp and | High in |
| FL; BCL6 | | | membrane protein | Alizadeh | DLBCL; ? |
| Translocation | | | | Common | |
| Partners | | | | Genes; | |
| | | | | Akasaka: | |
| | | | | Translocation | |
| | | | | partner of | |
| | | | | BCL6 but exp | |
| | | | | data not | |
| | | | | shown | |
| CLL vs. | NM_000633 | BCL2 | B-cell CLL/lymphoma 2 | CLL | High in CLL |
| DLBCL | | | | signature vs. | |
| | | | | DLBCL | |
| DLBCL Vs. | NM_005582 | LY64 | lymphocyte antigen 64 | Genes | Down in |
| FL | | | homolog, radioprotective | specific to | DLBCL, Up in |
| | | | 105 kDa (mouse) | either | FL |
| | | | | DLBCL or | |
| | | | | FL | |
| DLBCL Vs. | NM_006851 | GLIPR1 | GLI pathogenesis-related | Genes | Down in |
| FL | | | 1 (glioma) | specific to | DLBCL, Up in |
| | | | | either | FL |
| | | | | DLBCL or | |
| | | | | FL | |
| DLBCL Vs. | NM_001891 | CSN2 | casein beta | Genes | Down in |
| FL | | | | specific to | DLBCL, Up in |
| | | | | either | FL |
| | | | | DLBCL or | |
| | | | | FL | |
| DLBCL Vs. | NM_001781 | CD69 | CD69 antigen (p60, | Genes | Down in |
| FL | | | early T-cell activation | specific to | DLBCL, Up in |
| | | | antigen) | either | FL |
| | | | | DLBCL or | |
| | | | | FL | |
| CLL vs. | NM_005449 | TOSO | regulator of Fas-induced | Resting B-cell | High in CLL |
| DLBCL | | | apoptosis | signature | |
| DLBCL Vs. | NM_012203 | GRHPR | glyoxylate | Expression in | Up in |
| FL; BCL6 | | | reductase/hydroxypyruvate | FL to DLBCL | transformation |
| Translocation | | | reductase | transformation; | from FL to |
| Partners | | | | Translocation | DLBCL |
| | | | | partner of | |
| | | | | BCL6 but exp | |
| | | | | data not | |
| | | | | shown | |
| CLL vs. | NM_014207 | CD5 | CD5 antigen (p56-62) | Hs.58685 | High in CLL |
| DLBCL | | | | | |
| DLBCL Vs. | NM_004310 | ARHH | ras homolog gene | Genes | Down in |
| FL; BCL6 | | | family, member H | specific to | DLBCL, Up in |
| Translocation | | | | either | FL; ? |
| Partners | | | | DLBCL or | |
| | | | | FL; Shipp and | |
| | | | | Alizadeh | |
| | | | | Common | |
| | | | | Genes; | |
| | | | | Akasaka: | |
| | | | | Translocation | |
| | | | | partner of | |
| | | | | BCL6 but exp | |
| | | | | data not | |
| | | | | shown | |
| DLBCL Vs. | NM_002266 | KPNA2 | karyopherin aHCPha 2 | Genes | Up in |
| FL | | | (RAG cohort 1, importin | specific to | transformation |
| | | | aHCPha 1) | either | from FL to |
| | | | | DLBCL or | DLBCL |
| | | | | FL; | |
| | | | | Expression in | |
| | | | | FL to DLBCL | |
| | | | | transformation | |
| DLBCL Vs. | NM_007146 | ZNF161 | zinc finger protein 161 | Expression in | Up in FL |
| FL | | | | Fl vs. GC | |
| | | | | DLBCL | |
| DLBCL Vs. | NM_002228 | JUN | v-jun sarcoma virus 17 | Expression in | Up in FL |
| FL | | | oncogene homolog | Fl vs. GC | |
| | | | (avian) | DLBCL | |
| FL vs. | NM_000633 | BCL2 | B-cell CLL/lymphoma 2 | Genes | High in FL; |
| Burkitts; | | | | significantly | Up in FL |
| DLBCL Vs. | | | | deregulated; | |
| FL | | | | Genes up | |
| | | | | regulated in | |
| | | | | FL; | |
| | | | | Prognostically | |
| | | | | significant | |
| CLL vs. | NM_032663 | USP30 | ubiquitin specific | CLL | High in CLL |
| DLBCL | | | protease 30 | signature vs. | |
| | | | | DLBCL | |
| DLBCL Vs. | NM_002046 | GAPD | glyceraldehyde-3- | Genes | Up in |
| FL | | | phosphate | Correlated | transformation |
| | | | dehydrogenase | with Survival; | from FL to |
| | | | | Expression in | DLBCL |
| | | | | FL to DLBCL | |
| | | | | transformation | |
| DLBCL Vs. | NM_001197 | BIK | BCL2-interacting killer | Expression in | Down in FL |
| FL | | | (apoptosis-inducing) | Fl vs. GC | |
| | | | | DLBCL | |
| DLBCL Vs. | NM_002168 | IDH2 | isocitrate dehydrogenase | Genes | Up in DLBCL; |
| FL | | | 2 (NADP+), | specific to | Down in FL; |
| | | | mitochondrial | either | Up in |
| | | | | DLBCL or | transformation |
| | | | | FL; | from FL to |
| | | | | Expression in | DLBCL |
| | | | | FL to DLBCL | |
| | | | | transformation | |
| DLBCL Vs. | NM_004044 | ATIC | 5-aminoimidazole-4- | Genes | Up in DLBCL; |
| FL | | | carboxamide | specific to | Down in FL; |
| | | | ribonucleotide | either | Up in |
| | | | formyltransferase/IMP | DLBCL or | transformation |
| | | | cyclohydrolase | FL; | from FL to |
| | | | | Expression in | DLBCL |
| | | | | FL to DLBCL | |
| | | | | transformation | |
| DLBCL Vs. | NM_003403 | YY1 | YY1 transcription factor | Expression in | Down in FL |
| FL | | | | Fl vs. GC | |
| | | | | DLBCL | |
| DLBCL Vs. | NM_002910 | RENBP | renin binding protein | Expression in | Down in FL |
| FL | | | | Fl vs. GC | |
| | | | | DLBCL | |
| DLBCL Vs. | NM_000852 | GSTP1 | glutathione S-transferase | Expression in | Down in FL |
| FL | | | pi | Fl vs. GC | |
| | | | | DLBCL | |
| CLL vs. | NM_006495 | EVI2B | ecotropic viral | Resting B-cell | High in CLL |
| DLBCL | | | integration site 2B | signature | |
| DLBCL Vs. | NM_033306 | CASP4 | caspase 4, apoptosis- | Expression in | Down in FL |
| FL | | | related cysteine protease | Fl vs. GC | |
| | | | | DLBCL | |
| DLBCL Vs. | NM_006636 | MTHFD2 | methylene | Genes | Up in DLBCL; |
| FL | | | tetrahydrofolate | specific to | Down in FL; |
| | | | dehydrogenase (NAD+ | either | Up in |
| | | | dependent), | DLBCL or | transformation |
| | | | methenyltetrahydrofolate | FL; | from FL to |
| | | | cyclohydrolase | Expression in | DLBCL |
| | | | | FL to DLBCL | |
| | | | | transformation | |
| FL vs. | NM_002086 | GRB2 | growth factor receptor- | Genes | High in FL; |
| Burkitts; | | | bound protein 2 | significantly | Up in FL |
| DLBCL Vs. | | | | deregulated | |
| FL | | | | | |
| DLBCL Vs. | NM_058197 | CDKN2A | cyclin-dependent kinase | Expression in | Up in FL |
| FL; | | | inhibitor 2A (melanoma, | Fl vs. GC | |
| Deletions | | | p16, inhibits CDK4) | DLBCL | |
| associated | | | | | |
| with FL | | | | | |
| DLBCL Vs. | NM_002574 | PRDX1 | peroxiredoxin 1 | Genes | Up in DLBCL; |
| FL | | | | specific to | Down in FL |
| | | | | either | |
| | | | | DLBCL or | |
| | | | | FL | |
| DLBCL Vs. | NM_031966 | CCNB1 | cyclin B1 | Genes | Up in DLBCL; |
| FL | | | | specific to | Down in FL |
| | | | | either | |
| | | | | DLBCL or | |
| | | | | FL | |
| DLBCL Vs. | NM_002467 | MYC | v-myc myelocytomatosis | Signature | Uncertain in |
| FL | | | viral oncogene homolog | genes | Common |
| | | | (avian) | common to | Genes; Down |
| | | | | both Shipp | in |
| | | | | and Alizadeh | transformation |
| | | | | | from FL to |
| | | | | | DLBCL; Up in |
| | | | | | FL |
| CLL vs. | NM_001775 | CD38 | CD38 antigen (p45) | Hs.174944; | Low in CLL; |
| DLBCL | | | | Prognostically | High in G- |
| | | | | significant | center DLBCL |
| DLBCL Vs. | NM_004907 | ETR101 | immediate early protein | Expression in | Down in FL |
| FL | | | | Fl vs. GC | |
| | | | | DLBCL | |
| DLBCL Vs. | NM_000418 | IL4R | interleukin 4 receptor | Expression in | Up in FL |
| FL | | | | Fl vs. GC | |
| | | | | DLBCL | |
| DLBCL Vs. | NM_001344 | DAD1 | defender against cell | Expression in | Down in FL |
| FL | | | death 1 | Fl vs. GC | |
| | | | | DLBCL | |
| CLL vs. | NM_016187 | BIN2 | bridging integrator 2 | Resting B-cell | High in CLL |
| DLBCL | | | | signature | |
| CLL vs. | NM_001197 | BIK | BCL2-interacting killer | High in G- | Low in CLL; |
| DLBCL | | | (apoptosis-inducing) | center type | High in G- |
| | | | | DLBCL | center DLBCL |
| CLL vs. | NM_001706 | BCL6 | B-cell CLL/lymphoma 6 | High in G- | Low in CLL; |
| DLBCL | | | (zinc finger protein 51) | center type | High in G- |
| | | | | DLBCL | center DLBCL |
| CLL vs. | NM_000698 | ALOX5 | arachidonate 5- | Resting B-cell | High in CLL |
| DLBCL | | | lipoxygenase | signature | |
| CLL vs. | NM_002390 | ADAM11 | a disintegrin and | Resting B-cell | High in CLL |
| DLBCL | | | metalloproteinase | signature | |
| | | | domain 11 | | |
| DLBCL Vs. | NM_000884 | IMPDH2 | IMP (inosine | Genes | Up in DLBCL; |
| FL | | | monophosphate) | specific to | Down in FL; |
| | | | dehydrogenase 2 | either | Up in |
| | | | | DLBCL or | transformation |
| | | | | FL; | from FL to |
| | | | | Expression in | DLBCL |
| | | | | FL tp DLBCL | |
| | | | | transformation | |
| DLBCL Vs. | NM_002511 | NMBR | neuromedin B receptor | Expression in | Up in FL |
| FL | | | | Fl vs. GC | |
| | | | | DLBCL | |
| DLBCL Vs. | NM_005566 | LDHA | lactate dehydrogenase A | Genes | Up in DLBCL; |
| FL | | | | specific to | Down in FL; |
| | | | | either | Up in |
| | | | | DLBCL or | transformation |
| | | | | FL; | from FL to |
| | | | | Expression in | DLBCL |
| | | | | FL to DLBCL | |
| | | | | transformation | |
| DLBCL Vs. | NM_002166 | ID2 | inhibitor of DNA | Expression in | Up in FL |
| FL | | | binding 2, dominant | Fl vs. GC | |
| | | | negative helix-loop-helix | DLBCL | |
| | | | protein | | |
| DLBCL Vs. | NM_005526 | HSF1 | heat shock transcription | Expression in | Up in FL |
| FL | | | factor 1 | Fl vs. GC | |
| | | | | DLBCL | |
| DLBCL Vs. | NM_001901 | CTGF | connective tissue growth | Expression in | Up in FL |
| FL | | | factor | Fl vs. GC | |
| | | | | DLBCL | |
| DLBCL Vs. | NM_000365 | TPI1 | triosephosphate | Genes | Up in DLBCL; |
| FL | | | isomerase 1 | specific to | Down in FL; |
| | | | | either | Up in |
| | | | | DLBCL or | transformation |
| | | | | FL; | from FL to |
| | | | | Expression in | DLBCL |
| | | | | FL to DLBCL | |
| | | | | transformation | |
| DLBCL Vs. | NM_000269 | NME1 | non-metastatic cells 1, | Genes | Up in DLBCL; |
| FL | | | protein (NM23A) | specific to | Down in FL; |
| | | | expressed in | either | Up in |
| | | | | DLBCL or | transformation |
| | | | | FL; | from FL to |
| | | | | Expression in | DLBCL |
| | | | | FL to DLBCL | |
| | | | | transformation | |
| CLL vs. | NM_005214 | CTLA4 | cytotoxic T-lymphocyte- | CLL | High in CLL |
| DLBCL | | | associated protein 4 | signature vs. | |
| | | | | DLBCL | |
| DLBCL Vs. | NM_003070 | SMARCA2 | SWI/SNF related, matrix | Expression in | Up in FL |
| FL | | | associated, actin | Fl vs. GC | |
| | | | dependent regulator of | DLBCL | |
| | | | chromatin, subfamily a, | | |
| | | | member 2 | | |
| ABC VS | NM_002648 | PIM1 | pim-1 oncogene | Wright | Low in GC |
| GC | | | | predictor; | DLBCL; Up in |
| DLBCL; | | | | Expression in | Transformation |
| DLBCL Vs. | | | | FL to DLBCL | |
| FL | | | | transformation | |
| DLBCL Vs. | NM_021643 | TRB2 | tribbles homolog 2 | Genes | Down in |
| FL | | | | specific to | DLBCL, Up in |
| | | | | either | FL |
| | | | | DLBCL or | |
| | | | | FL | |
| Survival; | NM_021603 | FXYD2 | FXYD domain | Genes | Up in cured; |
| DLBCL Vs. | | | containing ion transport | Correlated | Down in fatal; |
| FL | | | regulator 2 | with Survival; | Up in |
| | | | | Expression in | Transformed |
| | | | | FL to DLBCL | DLBCL |
| | | | | transformation | |
| DLBCL Vs. | NM_004619 | TRAF5 | TNF receptor-associated | Genes | Down in |
| FL; MLBCL | | | factor 5 | specific to | DLBCL, Up in |
| vs. DLBCL | | | | either | FL; Low in |
| | | | | DLBCL or | MLBCL |
| | | | | FL | |
| DLBCL Vs. | NM_005998 | CCT3 | chaperonin containing | Genes | Up in DLBCL; |
| FL | | | TCP1, subunit 3 | specific to | Down in FL; |
| | | | (gamma) | either | Up in |
| | | | | DLBCL or | Transformation |
| | | | | FL; | |
| | | | | Expression in | |
| | | | | FL to DLBCL | |
| | | | | transformation | |
| DLBCL Vs. | NM_000034 | ALDOA | aldolase A, fructose- | Genes | Up in DLBCL; |
| FL | | | bisphosphate | specific to | Down in FL; |
| | | | | either | Up in |
| | | | | DLBCL or | Transformation |
| | | | | FL; | |
| | | | | Expression in | |
| | | | | FL to DLBCL | |
| | | | | transformation | |
| DLBCL vs. | NM_019841 | TRPV5 | transient receptor | Strong | Down in |
| MCL; | | | potential cation channel, | Classifier | DLBCL; Up in |
| CD5+/− | | | subfamily V, member 5 | Separating | MCL; Down |
| DLBCL | | | | MCLs and | in CD5+; Up |
| | | | | DLBCLs and | in CD5− |
| | | | | CD5+ from | |
| | | | | CD5− | |
| | | | | DLBCLs | |
| DLBCL Vs. | NM_002162 | ICAM3 | intercellular adhesion | Expression in | Down in |
| FL; DLBCL | | | molecule 3 | FL to DLBCL | Transformed |
| vs. MCL | | | | transformation; | DLBCL; |
| | | | | Strong | Down in |
| | | | | Classifier | DLBCL; Up in |
| | | | | Separating | MCL |
| | | | | MCLs and | |
| | | | | DLBCLs | |
| DLBCL Vs. | NM_001901 | CTGF | connective tissue growth | Expression in | Down in |
| FL | | | factor | FL to DLBCL | Transformed |
| | | | | transformation | DLBCL |
| ABC VS | NM_004630 | SF1 | splicing factor 1 | Signature | Uncertain in |
| GC DLBCL | | | | genes | Common |
| | | | | common to | Genes |
| | | | | both Shipp | |
| | | | | and Alizadeh | |
| ABC VS | NM_000418 | IL4R | interleukin 4 receptor | Signature | Uncertain in |
| GC DLBCL | | | | genes | Common |
| | | | | common to | Genes |
| | | | | both Shipp | |
| | | | | and Alizadeh | |
| ABC VS | NM_001882 | CRHBP | corticotropin releasing | Signature | Uncertain in |
| GC DLBCL | | | hormone binding protein | genes | Common |
| | | | | common to | Genes |
| | | | | both Shipp | |
| | | | | and Alizadeh | |
| ABC VS | NM_001775 | CD38 | CD38 antigen (p45) | Signature | Uncertain in |
| GC DLBCL | | | | genes | Common |
| | | | | common to | Genes |
| | | | | both Shipp | |
| | | | | and Alizadeh | |
| ABC VS | NM_003656 | CAMK1 | calcium/calmodulin- | Signature | Uncertain in |
| GC DLBCL | | | dependent protein kinase I | genes | Common |
| | | | | common to | Genes |
| | | | | both Shipp | |
| | | | | and Alizadeh | |
| Survival; | NM_002046 | GAPD | glyceraldehyde-3- | Genes | Down in |
| DLBCL Vs. | | | phosphate | Correlated | Cured; Up in |
| FL | | | dehydrogenase | with Survival; | Fatal; Up in |
| | | | | Expression in | Transformed |
| | | | | FL to DLBCL | DLBCL |
| | | | | transformation | |
| DLBCL Vs. | NM_000598 | IGFBP3 | insulin-like growth | Expression in | Down in |
| FL | | | factor binding protein 3 | FL to DLBCL | Transformed |
| | | | | transformation | DLBCL |
| MLBCL vs. | NM_002447 | MST1R | macrophage stimulating | MLBCL vs | High in |
| DLBCL | | | 1 receptor (c-met-related | DLBCL | MLBCL |
| | | | tyrosine kinase) | signature | |
| DLBCL vs. | NM_156039 | CSF3R | colony stimulating factor | Strong | Down in |
| MCL | | | 3 receptor (granulocyte) | Classifier | DLBCL; Up in |
| | | | | Separating | MCL |
| | | | | MCLs and | |
| | | | | DLBCLs | |
| DLBCL vs. | NM_181430 | ILF1 | interleukin enhancer | Strong | Down in |
| MCL | | | binding factor 1 | Classifier | DLBCL; Up in |
| | | | | Separating | MCL |
| | | | | MCLs and | |
| | | | | DLBCLs | |
| DLBCL vs. | NM_004475 | FLOT2 | flotillin 2 | Strong | Down in |
| MCL | | | | Classifier | DLBCL; Up in |
| | | | | Separating | MCL |
| | | | | MCLs and | |
| | | | | DLBCLs | |
| DLBCL vs. | NM_004941 | DHX8 | DEAH (Asp-Glu-Ala- | Strong | Down in |
| MCL | | | His) box polypeptide 8 | Classifier | DLBCL; Up in |
| | | | | Separating | MCL |
| | | | | MCLs and | |
| | | | | DLBCLs | |
| DLBCL vs. | NM_053056 | CCND1 | cyclin D1 (PRAD1: | Strong | Down in |
| MCL | | | parathyroid | Classifier | DLBCL; Up in |
| | | | adenomatosis 1) | Separating | MCL |
| | | | | MCLs and | |
| | | | | DLBCLs | |
| x (DLBCL) | NM_002738 | PRKCB1 | protein kinase C, beta 1 | Genes | Down in |
| | | | | Correlated | Cured; Up in |
| | | | | with Survival; | Fatal; Low in |
| | | | | MLBCL vs. | MLBCL |
| | | | | DLBCL | |
| Survival | NM_058197 | CDKN2A | cyclin-dependent kinase | Genes | Down in |
| (DLBCL) | | | inhibitor 2A (melanoma, | Correlated | Cured; Up in |
| | | | p16, inhibits CDK4) | with Survival | Fatal |
| ABC VS | BC011857 | IGHM | immunoglobulin heavy | Wright | Low in GC |
| GC | | | constant mu | predictor; | DLBCL; Low |
| DLBCL; | | | | Expression in | in MLBCL; |
| MLBCL vs. | | | | FL to DLBCL | Down in |
| DLBCL; | | | | transformation | Transformed |
| DLBCL Vs. | | | | | DLBCL |
| FL | | | | | |
| MLBCL vs. | NM_004972 | JAK2 | Janus kinase 2 (a protein | MLBCL vs | High in |
| DLBCL | | | tyrosine kinase) | DLBCL | MLBCL |
| | | | | signature | |
| MLBCL vs. | NM_006889 | CD86 | CD86 antigen (CD28 | MLBCL vs | High in |
| DLBCL | | | antigen ligand 2, B7-2 | DLBCL | MLBCL |
| | | | antigen) | signature | |
| MLBCL vs. | NM_001715 | BLK | B lymphoid tyrosine | MLBCL vs | Low in |
| DLBCL | | | kinase | DLBCL | MLBCL |
| | | | | signature | |
| DLBCL Vs. | NM_012203 | GRHPR | glyoxylate | Expression in | Up in |
| FL | | | reductase/hydroxypyruvate | FL to DLBCL | Transformation |
| | | | reductase | transformation | |
| DLBCL Vs. | NM_003070 | SMARCA 2 | SWI/SNF related, matrix | Expression in | Down in |
| FL | | | associated, actin | FL to DLBCL | Transformed |
| | | | dependent regulator of | transformation | DLBCL |
| | | | chromatin, subfamily a, | | |
| | | | member 2 | | |
| DLBCL Vs. | NM_004907 | ETR101 | immediate early protein | Expression in | Down in |
| FL | | | | FL to DLBCL | Transformed |
| | | | | transformation | DLBCL |
| DLBCL vs. | NM_134269 | SMTN | smoothelin | Strong | Down in |
| MCL | | | | Classifier | DLBCL; Up in |
| | | | | Separating | MCL |
| | | | | MCLs and | |
| | | | | DLBCLs | |
| DLBCL Vs. | NM_000405 | GM2A | GM2 ganglioside | Genes | Up in DLBCL; |
| FL | | | activator protein | specific to | Down in FL |
| | | | | either | |
| | | | | DLBCL or | |
| | | | | FL | |
| ABC VS | NM_002460 | IRF4 | interferon regulatory | Wright | Low in GC |
| GC | | | factor 4, MUM1 | predictor; | DLBCL; |
| DLBCL; | | | | Signature | Uncertain in |
| DLBCL Vs. | | | | genes | Common |
| FL | | | | common to | Genesin |
| | | | | both Shipp | Common |
| | | | | and Alizadeh; | Genes; Up in |
| | | | | Expression in | Transformed |
| | | | | FL to DLBCL | DLBCL |
| | | | | transformation | |
| ABC VS | NM_002467 | MYC | v-myc myelocytomatosis | Signature | Uncertain in |
| GC | | | viral oncogene homolog | genes | Common |
| DLBCL; | | | (avian) | common to | Genes; Down |
| DLBCL Vs. | | | | both Shipp | in Cured; Up |
| FL | | | | and Alizadeh; | in Fatal; Down |
| | | | | Rosenwald | in |
| | | | | Survival | Transformation; |
| | | | | Predictor | Up in |
| | | | | Genes | Transformed |
| | | | | | DLBCL, de |
| | | | | | Vos |
| DLBCL Vs. | NM_006325 | RAN | RAN, member RAS | Expression in | Up in |
| FL | | | oncogene family | FL to DLBCL | transformation |
| | | | | transformation | from FL to |
| | | | | | DLBCL |
| DLBCL Vs. | NM_002129 | HMGB2 | high-mobility group box 2 | Expression in | Up in |
| FL | | | | FL to DLBCL | transformation |
| | | | | transformation | from FL to |
| | | | | | DLBCL |
| DLBCL Vs. | NM_005340 | HINT1 | histidine triad nucleotide | Expression in | Up in |
| FL | | | binding protein 1 | FL to DLBCL | transformation |
| | | | | transformation | from FL to |
| | | | | | DLBCL |
| DLBCL Vs. | NM_006472 | TXNIP | thioredoxin interacting | Genes | Down in |
| FL | | | protein | specific to | DLBCL, Up in |
| | | | | either | FL; Down in |
| | | | | DLBCL or | Transformed |
| | | | | FL; | DLBCL |
| | | | | Expression in | |
| | | | | FL to DLBCL | |
| | | | | transformation | |
| DLBCL Vs. | NM_001783 | CD79A | CD79A antigen | Expression in | Up in |
| FL | | | (immunoglobulin- | FL to DLBCL | transformation |
| | | | associated aHCPha) | transformation | from FL to |
| | | | | | DLBCL |
| ABC VS | NM_001706 | BCL6 | B-cell CLL/lymphoma 6 | Wright | High in GC |
| GC DLBCL | | | (zinc finger protein 51) | predictor; | DLBCL; |
| | | | | Signature | Uncertain in |
| | | | | genes | Common |
| | | | | common to | Genesin |
| | | | | both Shipp | Common |
| | | | | and Alizadeh; | Genes; Up in |
| | | | | Rosenwald | cured; Down |
| | | | | Survival | in fatal |
| | | | | Predictor | |
| | | | | Genes | |
| DLBCL Vs. | NM_002648 | PIM1 | pim-1 oncogene | Wright | Low in GC |
| FL | | | | predictor; | DLBCL; Up in |
| | | | | Expression in | transformation |
| | | | | FL to DLBCL | from FL to |
| | | | | transformation | DLBCL |
| DLBCL Vs. | NM_021960 | Mcl-1 | myeloid cell leukemia | Genes up | High in FL |
| FL | | | sequence 1 (BCL2- | regulated in | |
| | | | related) (MCL1) | FL | |
| DLBCL Vs. | NM_002838 | PTPRC | protein tyrosine | Expression in | Down in |
| FL | | | phosphatase, receptor | FL to DLBCL | transformation |
| | | | type, C | transformation | from FL to |
| | | | | | DLBCL |
| DLBCL Vs. | NM_152866 | MS4A1 | membrane-spanning 4- | Expression in | Down in |
| FL | | | domains, subfamily A, | FL to DLBCL | transformation |
| | | | member 1 | transformation | from FL to |
| | | | | | DLBCL |
| DLBCL Vs. | NM_006495 | EVI2B | ecotropic viral | Expression in | Down in |
| FL | | | integration site 2B | FL to DLBCL | transformation |
| | | | | transformation | from FL to |
| | | | | | DLBCL |
| DLBCL Vs. | NM_001647 | APOD | apolipoprotein D | Expression in | Down in |
| FL | | | | FL to DLBCL | transformation |
| | | | | transformation | from FL to |
| | | | | | DLBCL |
| DLBCL Vs. | NM_000626 | CD79B | CD79B antigen | Expression in | Up in |
| FL | | | (immunoglobulin- | FL to DLBCL | transformation |
| | | | associated beta) | transformation | from FL to |
| | | | | | DLBCL |
| ABC VS | NM_001092 | ABR | active BCR-related gene | Signature | Uncertain in |
| GC | | | | genes | Common |
| DLBCL; | | | | common to | Genes; Down |
| DLBCL vs. | | | | both Shipp | in DLBCL; Up |
| MCL | | | | and Alizadeh; | in MCL |
| | | | | Strong | |
| | | | | Classifier | |
| | | | | Separating | |
| | | | | MCLs and | |
| | | | | DLBCLs | |
| DLBCL vs. | NM_000277 | PAH | phenylalanine | Strong | Down in |
| MCL | | | hydroxylase | Classifier | DLBCL; Up in |
| | | | | Separating | MCL |
| | | | | MCLs and | |
| | | | | DLBCLs | |
| ABC VS | NM_000698 | ALOX5 | arachidonate 5- | Signature | Uncertain in |
| GC | | | lipoxygenase | genes | Common |
| DLBCL; | | | | common to | Genes; Down |
| DLBCL Vs. | | | | both Shipp | in |
| FL | | | | and Alizadeh; | Transformed |
| | | | | Expression in | DLBCL |
| | | | | FL to DLBCL | |
| | | | | transformation | |
| ABC VS | XM_034274 | MYBL1 | v-myb myeloblastosis | Wright | High in GC |
| GC DLBCL | | | viral oncogene homolog | predictor; | DLBCL; |
| | | | (avian)-like 1 | Signature | Uncertain in |
| | | | | genes | Common |
| | | | | common to | Genesin |
| | | | | both Shipp | Common |
| | | | | and Alizadeh | Genes |
| ABC VS | NM_006152 | LRMP | lymphoid-restricted | Wright | High in GC |
| GC DLBCL | | | membrane protein | predictor; | DLBCL; |
| | | | | Signature | Uncertain in |
| | | | | genes | Common |
| | | | | common to | Genesin |
| | | | | both Shipp | Common |
| | | | | and Alizadeh | Genes |
| ABC VS | NM_005574 | LMO2 | LIM domain only 2 | Wright | High in GC |
| GC DLBCL | | | (rhombotin-like 1) | predictor; | DLBCL; |
| | | | | Signature | Uncertain in |
| | | | | genes | Common |
| | | | | common to | Genesin |
| | | | | both Shipp | Common |
| | | | | and Alizadeh | Genes |
| ABC VS | NM_001987 | ETV6 | ets variant gene 6 (TEL | Wright | Low in GC |
| GC DLBCL | | | oncogene) | predictor; | DLBCL; |
| | | | | Signature | Uncertain in |
| | | | | genes | Common |
| | | | | common to | Genesin |
| | | | | both Shipp | Common |
| | | | | and Alizadeh | Genes |
| ABC VS | NM_001242 | TNTFRSF7 | tumor necrosis factor | Signature | Uncertain in |
| GC | | | receptor superfamily, | genes | Common |
| DLBCL; | | | member 7 | common to | Genes; Low in |
| MLBCL vs. | | | | both Shipp | MLBCL; |
| DLBCL; | | | | and Alizadeh; | Down in |
| DLBCL Vs. | | | | MLBCL vs. | Transformed |
| FL | | | | DLBCL; | DLBCL |
| | | | | Expression in | |
| | | | | FL to DLBCL | |
| | | | | transformation | |
| ABC VS | NM_002166 | ID2 | inhibitor of DNA | Signature | Uncertain in |
| GC DLBCL | | | binding 2, dominant | genes | Common |
| | | | negative helix-loop-helix | common to | Genes; Up in |
| | | | protein | both Shipp | CD5+; Down |
| | | | | and Alizadeh; | in CD5− |
| | | | | Strong | |
| | | | | Classifier | |
| | | | | Genes | |
| | | | | Separating | |
| | | | | CD5+ from | |
| | | | | CD5− | |
| | | | | DLBCLs | |
| ABC VS | NM_000633 | BCL2 | B-cell CLL/lymphoma 2 | Signature | Uncertain in |
| GC | | | | genes | Common |
| DLBCL; | | | | common to | Genes; Down |
| DLBCL Vs. | | | | both Shipp | in |
| FL | | | | and Alizadeh | Transformed |
| | | | | | DLBCL |
| MLBCL vs. | NM_003254 | TIMP1 | tissue inhibitor of | MLBCL vs | High in |
| DLBCL | | | metalloproteinase 1 | DLBCL | MLBCL |
| | | | (erythroid potentiating | signature | |
| | | | activity, collagenase | | |
| | | | inhibitor) | | |
| DLBCL Vs. | NM_006184 | NUCB1 | nucleobindin 1 | Expression in | Up in |
| FL | | | | FL to DLBCL | Transformed |
| | | | | transformation | DLBCL |
| ABC VS | NM_007289 | MME | membrane metallo- | Wright | High in GC |
| GC | | | endopeptidase (neutral | predictor; | DLBCL; |
| DLBCL; | | | endopeptidase, | Signature | Uncertain in |
| MLBCL vs. | | | enkephalinase, CALLA, | genes | Common |
| DLBCL | | | CD10) | common to | Genes; Up in |
| | | | | both Shipp | cured; Down |
| | | | | and Alizadeh; | in fatal; Low |
| | | | | MLBCL vs. | in MLBCL |
| | | | | DLBCL | |
| | | | | signature | |
| ABC VS | NM_004513 | IL16 | interleukin 16 | Wright | Low in GC |
| GC | | | (lymphocyte | predictor; | DLBCL; |
| DLBCL; | | | chemoattractant factor) | Signature | Uncertain in |
| MLBCL vs. | | | | genes | Common |
| DLBCL | | | | common to | Genes; Low in |
| | | | | both Shipp | MLBCL |
| | | | | and Alizadeh; | |
| | | | | MLBCL vs. | |
| | | | | DLBCL | |
| | | | | signature | |
| ABC VS | NM_001877 | CR2 | complement component | Signature | Uncertain in |
| GC | | | (3d/Epstein Barr virus) | genes | Common |
| DLBCL; | | | receptor 2 | common to | Genes; Down |
| DLBCL Vs. | | | | both Shipp | in |
| FL | | | | and Alizadeh; | Transformed |
| | | | | Expression in | DLBCL |
| | | | | FL to DLBCL | |
| | | | | transformation | |
| DLBCL Vs. | NM_001831 | CLU | clusterin (complement | Genes | Down in |
| FL | | | lysis inhibitor, SP-40,40, | specific to | DLBCL, Up in |
| | | | sulfated glycoprotein 2, | either | FL; Down in |
| | | | testosterone-repressed | DLBCL or | Transformed |
| | | | prostate message 2, | FL; | DLBCL |
| | | | apolipoprotein J) | Expression in | |
| | | | | FL to DLBCL | |
| | | | | transformation | |
| ABC VS | NM_001923 | DDB1 | damage-specific DNA | Wright | Low in GC |
| GC DLBCL | | | binding protein 1, | predictor; | DLBCL; |
| | | | 127 kDa | Signature | Uncertain in |
| | | | | genes | Common |
| | | | | common to | Genesin |
| | | | | both Shipp | Common |
| | | | | and Alizadeh | Genes |

TABLE 7
An HCP set specific for FL, according to one embodiment of the invention

| Feature | Accession | Symbol | Name | Notes | Level |
|---|---|---|---|---|---|
| FL | BC011857 | IGHM | immunoglobulin heavy | | Down in FL (−6.6) |
| | | | constant mu | | |
| FL; MCL | NM_000424 | KRT5 | keratin 5 (epidermolysis | | down in FL (−5.3); |
| | | | bullosa simplex, | | down in |
| | | | Dowling- | | MCL (−4) |
| | | | Meara/Kobner/Weber- | | |
| | | | Cockayne types) | | |
| FL vs MCL | AB037771 | USP53 | ubiquitin specific | | up in FL vs |
| | | | protease 53 | | MCL (4.2) |
| FL vs | NM_025113 | C13orf18 | chromosome 13 open | | down in FL vs |
| CLL/SLL | | | reading frame 18 | | SLL (−5.3) |
| FL vs MCL | NM_000418 | IL4R | interleukin 4 receptor | | up in FL vs |
| | | | | | MCL (5.4) |
| FL vs MCL | NM_001781 | CD69 | CD69 antigen (p60, | | down in FL vs |
| | | | early T-cell activation | | MCL (−4.1) |
| | | | antigen) | | |
| FL | NM_002984 | CCL4 | chemokine (C-C motif) | | down in FL (−5.6) |
| | | | ligand 4 | | |
| FL vs MCL | NM_000405 | GM2A | GM2 ganglioside | | down in FL vs |
| | | | activator | | MCL (−4.4) |
| FL vs MCL | NM_002228 | JUN | v-jun sarcoma virus 17 | | down in FL vs |
| | | | oncogene homolog | | MCL (−4.6) |
| | | | (avian) | | |
| FL | NM_006850 | IL24 | interleukin 24 | | down in FL (−8.1) |
| FL vs MCL | NM_006475 | POSTN | periostin, osteoblast | | Up in FL vs |
| | | | specific factor | | MCL (4) |
| FL vs SLL | NM_031942 | CDCA7 | cell division cycle | | Up in FL vs |
| | | | associated 7 | | SLL (8.8) |
| FL vs MCL | NM_001640 | APEH | N-acylaminoacyl-peptide | | down in MCL |
| | | | hydrolase | | (−6.5); up in |
| | | | | | FL vs MCL |
| | | | | | (6) |
| FL vs MCL | NM_018842 | LOC55971 | insulin receptor tyrosine | | down in FL vs |
| | | | kinase substrate | | MCL (−8.4) |
| FL | NM_147180 | PPP3R2 | protein phosphatase 3 | | down in FL (−4.4) |
| | | | (formerly 2B), | | |
| | | | regulatory subunit B, | | |
| | | | 19 kDa, beta isoform | | |
| | | | (calcineurin B, type II) | | |
| FL | NM_002276 | KRT19 | keratin 19 | | down in FL (−5) |
| FL | NM_001675 | ATF4 | activating transcription | | up in FL (4.4) |
| | | | factor 4 (tax-responsive | | |
| | | | enhancer element B67) | | |
| FL vs MCL | NM_021813 | BACH2 | BTB and CNC | | up in FL vs |
| | | | homology 1, basic | | MCL (5.5) |
| | | | leucine zipper | | |
| | | | transcription factor 2 | | |
| FL | NM_006565 | CTCF | CCCTC-binding factor | | up in FL vs |
| | | | (zinc finger protein) | | MCL (9.3); up |
| | | | | | in FL vs SLL |
| | | | | | (10.2) |
| FL | NM_005537 | ING1 | inhibitor of growth | | up in FL (4.5) |
| | | | family, member 1 | | |
| FL vs MCL | NM_006164 | NFE2L2 | nuclear factor (erythroid- | | up in FL vs |
| | | | derived 2)-like 2 | | MCL (4.1) |
| FL | NM_004630 | SF1 | splicing factor 1 | | down in FL (−4.2) |
| CLL/SLL vs | NM_019846 | CCL28 | chemokine (C-C motif) | | up in FL vs |
| FL | | | ligand 28 | | CLL/SLL |
| | | | | | (5.2) |
| FL vs MCL | NM_006889 | CD86 | CD86 antigen (CD28 | | up in FL vs |
| | | | antigen ligand 2, B7-2 | | MCL (7.9) |
| | | | antigen) | | |
| FL | NM_032738 | FREB | Fc receptor homolog | | up in FL (4.8) |
| | | | expressed in B cells | | |
| FL | BC019046 | IGHG1 | immunoglobulin heavy | | down in FL (−6.6) |
| | | | constant gamma 1 (G1m | | |
| | | | marker) | | |
| FL | NM_002189 | IL15RA | interleukin 15 receptor, | | down in FL (−4.1) |
| | | | aHCPha | | |
| FL | NM_004827 | ABCG2 | ATP-binding cassette, | | down in FL (−8.4) |
| | | | sub-family G (WHITE), | | |
| | | | member 2 | | |
| FLL | NM_001647 | APOD | apolipoprotein D | | down in FL vs |
| | | | | | MCL (−8.3); |
| | | | | | down in FL vs |
| | | | | | CLL/SLL (−5.8) |
| DLBCL Vs. | NM_002467 | MYC | v-myc myelocytomatosis | Signature | Uncertain in |
| FL | | | viral oncogene homolog | genes | Common |
| | | | (avain) | common to | Genes; Down |
| | | | | both Shipp | in |
| | | | | and Alizadeh | transformation |
| | | | | | from FL to |
| | | | | | DLBCL; Up in |
| | | | | | FL |
| DLBCL Vs. | NM_031966 | CCNB1 | cyclin B1 | Genes | Up in DLBCL; |
| FL | | | | specific to | Down in FL |
| | | | | either | |
| | | | | DLBCL or | |
| | | | | FL | |
| DLBCL Vs. | NM_002574 | PRDX1 | peroxiredoxin 1 | Genes | Up in DLBCL; |
| FL | | | | specific to | Down in FL |
| | | | | either | |
| | | | | DLBCL or | |
| | | | | FL | |
| DLBCL Vs. | NM_058197 | CDKN2A | cyclin-dependent kinase | Expression in | Up in FL |
| FL; | | | inhibitor 2A (melanoma, | Fl vs. GC | |
| Deletions | | | p16, inhibits CDK4) | DLBCL | |
| associated | | | | | |
| with FL | | | | | |
| FL vs. | NM_002086 | GRB2 | growth factor receptor- | Genes | High in FL; |
| Burkitts; | | | bound protein 2 | significantly | Up in FL |
| DLBCL Vs. | | | | deregulated | |
| FL | | | | | |
| DLBCL Vs. | NM_001197 | BIK | BCL2-interacting killer | Expression in | Down in FL |
| FL | | | (apoptosis-inducing) | Fl vs. GC | |
| | | | | DLBCL | |
| DLBCL Vs. | NM_033306 | CASP4 | caspase 4, apoptosis- | Expression in | Down in FL |
| FL | | | related cysteine protease | Fl vs. GC | |
| | | | | DLBCL | |
| DLBCL Vs. | NM_004907 | ETR101 | immediate early protein | Expression in | Down in FL |
| FL | | | | Fl vs. GC | |
| | | | | DLBCL | |
| DLBCL Vs. | NM_000852 | GSTP1 | glutathione S-transferase | Expression in | Down in FL |
| FL | | | pi | Fl vs. GC | |
| | | | | DLBCL | |
| DLBCL Vs. | NM_002910 | RENBP | renin binding protein | Expression in | Down in FL |
| FL | | | | Fl vs. GC | |
| | | | | DLBCL | |
| DLBCL Vs. | NM_003403 | YY1 | YY1 transcription factor | Expression in | Down in FL |
| FL | | | | Fl vs. GC | |
| | | | | DLBCL | |
| DLBCL Vs. | NM_004044 | ATIC | 5-aminoimidazole-4- | Genes | Up in DLBCL; |
| FL | | | carboxamide | specific to | Down in FL; |
| | | | ribonucleotide | either | Up in |
| | | | formyltransferase/IMP | DLBCL or | transformation |
| | | | cyclohydrolase | FL; | from FL to |
| | | | | Expression in | DLBCL |
| | | | | FL to DLBCL | |
| | | | | transformation | |
| DLBCL Vs. | NM_002168 | IDH2 | isocitrate dehydrogenase | Genes | Up in DLBCL; |
| FL | | | 2 (NADP+), | specific to | Down in FL; |
| | | | mitochondrial | either | Up in |
| | | | | DLBCL or | transformation |
| | | | | FL; | from FL to |
| | | | | Expression in | DLBCL |
| | | | | FL to DLBCL | |
| | | | | transformation | |
| DLBCL Vs. | NM_000884 | IMPDH2 | IMP (inosine | Genes | Up in DLBCL; |
| FL | | | monophosphate) | specific to | Down in FL; |
| | | | dehydrogenase 2 | either | Up in |
| | | | | DLBCL or | transformation |
| | | | | FL; | from FL to |
| | | | | Expression in | DLBCL |
| | | | | FL to DLBCL | |
| | | | | transformation | |
| DLBCL Vs. | NM_005566 | LDHA | lactate dehydrogenase A | Genes | Up in DLBCL; |
| FL | | | | specific to | Down in FL; |
| | | | | either | Up in |
| | | | | DLBCL or | transformation |
| | | | | FL; | from FL to |
| | | | | Expression in | DLBCL |
| | | | | FL to DLBCL | |
| | | | | transformation | |
| DLBCL Vs. | NM_006636 | MTHFD2 | methylene | Genes | Up in DLBCL; |
| FL | | | tetrahydrofolate | specific to | Down in FL; |
| | | | dehydrogenase (NAD+ | either | Up in |
| | | | dependent), | DLBCL or | transformation |
| | | | methenyltetrahydrofolate | FL; | from FL to |
| | | | cyclohydrolase | Expression in | DLBCL |
| | | | | FL to DLBCL | |
| | | | | transformation | |
| DLBCL Vs. | NM_000269 | NME1 | non-metastatic cells 1, | Genes | Up in DLBCL; |
| FL | | | protein (NM23A) | specific to | Down in FL; |
| | | | expressed in | either | Up in |
| | | | | DLBCL or | transformation |
| | | | | FL; | from FL to |
| | | | | Expression in | DLBCL |
| | | | | FL to DLBCL | |
| | | | | transformation | |
| DLBCL Vs. | NM_000365 | TPI1 | triosephosphate | Genes | Up in DLBCL; |
| FL | | | isomerase 1 | specific to | Down in FL; |
| | | | | either | Up in |
| | | | | DLBCL or | transformation |
| | | | | FL; | from FL to |
| | | | | Expression in | DLBCL |
| | | | | FL to DLBCL | |
| | | | | transformation | |
| DLBCL Vs. | NM_001901 | CTGF | connective tissue growth | Expression in | Up in FL |
| FL | | | factor | Fl vs. GC | |
| | | | | DLBCL | |
| DLBCL Vs. | NM_005526 | HSF1 | heat shock transcription | Expression in | Up in FL |
| FL | | | factor 1 | Fl vs. GC | |
| | | | | DLBCL | |
| DLBCL Vs. | NM_002166 | ID2 | inhibitor of DNA | Expression in | Up in FL |
| FL | | | binding 2, dominant | Fl vs. GC | |
| | | | negative helix-loop-helix | DLBCL | |
| | | | protein | | |
| DLBCL Vs. | NM_000418 | IL4R | interleukin 4 receptor | Expression in | Up in FL |
| FL | | | | Fl vs. GC | |
| | | | | DLBCL | |
| DLBCL Vs. | NM_002511 | NMBR | neuromedin B receptor | Expression in | Up in FL |
| FL | | | | Fl vs. GC | |
| | | | | DLBCL | |
| DLBCL Vs. | NM_003070 | SMARCA2 | SWI/SNF related, matrix | Expression in | Up in FL |
| FL | | | associated, actin | Fl vs. GC | |
| | | | dependent regulator of | DLBCL | |
| | | | chromatin, subfamily a, | | |
| | | | member 2 | | |
| FL vs. | NM_000633 | BCL2 | B-cell CLL/lymphoma 2 | Genes | High in FL; |
| Burkitts; | | | | significantly | Up in FL |
| DLBCL Vs. | | | | deregulated; | |
| FL | | | | Genes up | |
| | | | | regulated in | |
| | | | | FL; | |
| | | | | Prognostically | |
| | | | | significant | |
| DLBCL Vs. | NM_002228 | JUN | v-jun sarcoma virus 17 | Expression in | Up in FL |
| FL | | | oncogene homolog | Fl vs. GC | |
| | | | (avian) | DLBCL | |
| DLBCL Vs. | NM_007146 | ZNF161 | zinc finger protein 161 | Expression in | Up in FL |
| FL | | | | Fl vs. GC | |
| | | | | DLBCL | |
| DLBCL Vs. | NM_002266 | KPNA2 | karyopherin aHCPha 2 | Genes | Up in |
| FL | | | (RAG cohort 1, importin | specific to | transformation |
| | | | aHCPha 1) | either | from FL to |
| | | | | DLBCL or | DLBCL |
| | | | | FL; | |
| | | | | Expression in | |
| | | | | FL to DLBCL | |
| | | | | transformation | |
| DLBCL Vs. | NM_004310 | ARHH | ras homolog gene | Genes | Down in |
| FL; BCL6 | | | family, member H | specific to | DLBCL, Up in |
| Translocation | | | | either | FL; ? |
| Partners | | | | DLBCL or | |
| | | | | FL; Shipp and | |
| | | | | Alizadeh | |
| | | | | Common | |
| | | | | Genes; | |
| | | | | Akasaka: | |
| | | | | Translocation | |
| | | | | partner of | |
| | | | | BCL6 but exp | |
| | | | | data not | |
| | | | | shown | |
| DLBCL Vs. | NM_006152 | LRMP | lymphoid-restricted | Shipp and | High in |
| FL; BCL6 | | | membrane protein | Alizadeh | DLBCL; ? |
| Translocation | | | | Common | |
| Partners | | | | Genes; | |
| | | | | Akasaka: | |
| | | | | Translocation | |
| | | | | partner of | |
| | | | | BCL6 but exp | |
| | | | | data not | |
| | | | | shown | |
| DLBCL Vs. | NM_012203 | GRHPR | glyoxylate | Expression in | Up in |
| FL; BCL6 | | | reductase/hydroxypyruvate | FL to DLBCL | transformation |
| Translocation | | | reductase | transformation; | from FL to |
| Partners | | | | Translocation | DLBCL |
| | | | | partner of | |
| | | | | BCL6 but exp | |
| | | | | data not | |
| | | | | shown | |
| DLBCL Vs. | NM_002046 | GAPD | glyceraldehyde-3- | Genes | Up in |
| FL | | | phosphate | Correlated | transformation |
| | | | dehydrogenase | with Survival; | from FL to |
| | | | | Expression in | DLBCL |
| | | | | FL to DLBCL | |
| | | | | transformation | |
| DLBCL Vs. | NM_001781 | CD69 | CD69 antigen (p60, | Genes | Down in |
| FL | | | early T-cell activation | specific to | DLBCL, Up in |
| | | | antigen) | either | FL |
| | | | | DLBCL or | |
| | | | | FL | |
| DLBCL Vs. | NM_001891 | CSN2 | casein beta | Genes | Down in |
| FL | | | | specific to | DLBCL, Up in |
| | | | | either | FL |
| | | | | DLBCL or | |
| | | | | FL | |
| FL | NM_022829 | SLC13A3 | solute carrier family 13 | | up in FL (4.3) |
| | | | (sodium-dependent | | |
| | | | dicarboxylate | | |
| | | | transporter), member 3 | | |
| DLBCL Vs. | NM_006851 | GLIPR1 | GLI pathogenesis-related | Genes | Down in |
| FL | | | 1 (glioma) | specific to | DLBCL, Up in |
| | | | | either | FL |
| | | | | DLBCL or | |
| | | | | FL | |
| DLBCL Vs. | NM_005582 | LY64 | lymphocyte antigen 64 | Genes | Down in |
| FL | | | homolog, radioprotective | specific to | DLBCL, Up in |
| | | | 105 kDa (mouse) | either | FL |
| | | | | DLBCL or | |
| | | | | FL | |
| DLBCL Vs. | NM_021643 | TRB2 | tribbles homolog 2 | Genes | Down in |
| FL | | | | specific to | DLBCL, Up in |
| | | | | either | FL |
| | | | | DLBCL or | |
| | | | | FL | |
| DLBCL Vs. | NM_006472 | TXNIP | thioredoxin interacting | Genes | Down in |
| FL | | | protein | specific to | DLBCL, Up in |
| | | | | either | FL |
| | | | | DLBCL or | |
| | | | | FL | |
| DLBCL Vs. | NM_001344 | DAD1 | defender against cell | Expression in | Down in FL |
| FL | | | death 1 | Fl vs. GC | |
| | | | | DLBCL | |
| DLBCL Vs. | NM_001647 | APOD | apolipoprotein D | Expression in | Down in |
| FL | | | | FL to DLBCL | transformation |
| | | | | transformation | from FL to |
| | | | | | DLBCL |
| DLBCL Vs. | NM_006495 | EVI2B | ecotropic viral | Expression in | Down in |
| FL | | | integration site 2B | FL to DLBCL | transformation |
| | | | | transformation | from FL to |
| | | | | | DLBCL |
| DLBCL Vs. | NM_152866 | MS4A1 | membrane-spanning 4- | Expression in | Down in |
| FL | | | domains, subfamily A, | FL to DLBCL | transformation |
| | | | member 1 | transformation | from FL to |
| | | | | | DLBCL |
| DLBCL Vs. | NM_002838 | PTPRC | protein tyrosine | Expression in | Down in |
| FL | | | phosphatase, receptor | FL to DLBCL | transformation |
| | | | type, C | transformation | from FL to |
| | | | | | DLBCL |
| DLBCL Vs. | NM_021960 | Mcl-1 | myeloid cell leukemia | Genes up | High in FL |
| FL | | | sequence 1 (BCL2- | regulated in | |
| | | | related) (MCL1) | FL | |
| DLBCL Vs. | NM_002648 | PIM1 | pim-1 oncogene | Wright | Low in GC |
| FL | | | | predictor; | DLBCL; Up in |
| | | | | Expression in | transformation |
| | | | | FL to DLBCL | from FL to |
| | | | | transformation | DLBCL |
| DLBCL Vs. | NM_000405 | GM2A | GM2 ganglioside | Genes | Up in DLBCL; |
| FL | | | activator protein | specific to | Down in FL |
| | | | | either | |
| | | | | DLBCL or | |
| | | | | FL | |
| DLBCL Vs. | NM_001783 | CD79A | CD79A antigen | Expression in | Up in |
| FL | | | (immunoglobulin- | FL to DLBCL | transformation |
| | | | associated aHCPha) | transformation | from FL to |
| | | | | | DLBCL |
| DLBCL Vs. | NM_000626 | CD79B | CD79B antigen | Expression in | Up in |
| FL | | | (immunoglobulin- | FL to DLBCL | transformation |
| | | | associated beta) | transformation | from FL to |
| | | | | | DLBCL |
| CLL/SL vs | NM_006820 | C1orf29 | chromosome 1 open | | down in FL vs |
| FL | | | reading frame 29 | | MCL (−4.3) |
| DLBCL Vs. | NM_005340 | HINT1 | histidine triad nucleotide | Expression in | Up in |
| FL | | | binding protein 1 | FL to DLBCL | transformation |
| | | | | transformation | from FL to |
| | | | | | DLBCL |
| DLBCL Vs. | NM_002129 | HMGB2 | high-mobility group box 2 | Expression in | Up in |
| FL | | | | FL to DLBCL | transformation |
| | | | | transformation | from FL to |
| | | | | | DLBCL |
| DLBCL Vs. | NM_006325 | RAN | RAN, member RAS | Expression in | Up in |
| FL | | | oncogene family | FL to DLBCL | transformation |
| | | | | transformation | from FL to |
| | | | | | DLBCL |
| FL vs. | NM_000277 | PAH | phenylalanine | Genes | High in FL |
| Burkitts | | | hydroxylase | significantly | |
| | | | | deregulated | |
| FL vs. | NM_133480 | TADA3L | transcriptional adaptor 3 | Genes | High in FL |
| Burkitts | | | (NGG1 homolog, yeast)- | significantly | |
| | | | like | deregulated | |
| FL vs. | NM_003656 | CAMK1 | calcium/calmodulin- | Genes | High in FL |
| Burkitts | | | dependent protein kinase I | significantly | |
| | | | | deregulated | |
| FL vs. | NM_005248 | FGR | Gardner-Rasheed feline | Genes | Low in FL |
| Burkitts | | | sarcoma viral (v-fgr) | significantly | |
| | | | oncogene homolog | deregulated | |
| FL vs. | NM_002447 | MST1R | macrophage stimulating | Genes | Low in FL |
| Burkitts | | | 1 receptor (c-met-related | significantly | |
| | | | tyrosine kinase) | deregulated | |
| FL vs. | NM_003254 | TIMP1 | tissue inhibitor of | Genes | Low in FL |
| Burkitts | | | metalloproteinase 1 | significantly | |
| | | | (erythroid potentiating | deregulated | |
| | | | activity, collagenase | | |
| | | | inhibitor) | | |
| ABC VS | NM_002467 | MYC | v-myc myelocytomatosis | Signature | Uncertain in |
| GC | | | viral oncogene homolog | genes | Common |
| DLBCL; | | | (avian) | common to | Genes; Down |
| DLBCL Vs. | | | | both Shipp | in Cured; Up |
| FL | | | | and Alizadeh; | in Fatal; Down |
| | | | | Rosenwald | in |
| | | | | Survival | Transformation; |
| | | | | Predictor | Up in |
| | | | | Genes | Transformed |
| | | | | | DLBCL, de |
| | | | | | Vos |
| ABC VS | NM_002460 | IRF4 | interferon regulatory | Wright | Low in GC |
| GC | | | factor 4, MUM1 | predictor; | DLBCL; |
| DLBCL; | | | | Signature | Uncertain in |
| DLBCL Vs. | | | | genes | Common |
| FL | | | | common to | Genesin |
| | | | | both Shipp | Common |
| | | | | and Alizadeh; | Genes; Up in |
| | | | | Expression in | Transformed |
| | | | | FL to DLBCL | DLBCL |
| | | | | transformation | |
| ABC VS | NM_001242 | TNFRSF7 | tumor necrosis factor | Signature | Uncertain in |
| GC | | | receptor superfamily, | genes | Common |
| DLBCL; | | | member 7 | common to | Genes; Low in |
| MLBCL vs. | | | | both Shipp | MLBCL; |
| DLBCL; | | | | and Alizadeh; | Down in |
| DLBCL Vs. | | | | MLBCL vs. | Transformed |
| FL | | | | DLBCL; | DLBCL |
| | | | | Expression in | |
| | | | | FL to DLBCL | |
| | | | | transformation | |
| ABC VS | NM_000633 | BCL2 | B-cell CLL/lymphoma 2 | Signature | Uncertain in |
| GC | | | | genes | Common |
| DLBCL; | | | | common to | Genes; Down |
| DLBCL Vs. | | | | both Shipp | in |
| FL | | | | and Alizadeh | Transformed |
| | | | | | DLBCL |
| ABC VS | NM_001877 | CR2 | complement component | Signature | Uncertain in |
| GC | | | (3d/Epstein Barr virus) | genes | Common |
| DLBCL; | | | receptor 2 | common to | Genes; Down |
| DLBCL Vs. | | | | both Shipp | in |
| FL | | | | and Alizadeh; | Transformed |
| | | | | Expression in | DLBCL |
| | | | | FL to DLBCL | |
| | | | | transformation | |
| DLBCL Vs. | NM_006184 | NUCB1 | nucleobindin 1 | Expression in | Up in |
| FL | | | | FL to DLBCL | Transformed |
| | | | | transformation | DLBCL |
| ABC VS | NM_000698 | ALOX5 | arachidonate 5- | Signature | Uncertain in |
| GC | | | lipoxygenase | genes | Common |
| DLBCL; | | | | common to | Genes; Down |
| DLBCL Vs. | | | | both Shipp | in |
| FL | | | | and Alizadeh; | Transformed |
| | | | | Expression in | DLBCL |
| | | | | FL to DLBCL | |
| | | | | transformation | |
| ABC VS | BC011857 | IGHM | immunoglobulin heavy | Wright | Low in GC |
| GC | | | constant mu | predictor; | DLBCL; Low |
| DLBCL; | | | | Expression in | in MLBCL; |
| MLBCL vs. | | | | FL to DLBCL | Down in |
| DLBCL; | | | | transformation | Transformed |
| DLBCL Vs. | | | | | DLBCL |
| FL | | | | | |
| DLBCL Vs. | NM_001831 | CLU | clusterin (complement | Genes | Down in |
| FL | | | lysis inhibitor, SP-40,40, | specific to | DLBCL, Up in |
| | | | sulfated glycoprotein 2, | either | FL; Down in |
| | | | testosterone-repressed | DLBCL or | Transformed |
| | | | prostate message 2, | FL; | DLBCL |
| | | | apolipoprotein J) | Expression in | |
| | | | | FL to DLBCL | |
| | | | | transformation | |
| DLBCL Vs. | NM_006472 | TXNIP | thioredoxin interacting | Genes | Down in |
| FL | | | protein | specific to | DLBCL, Up in |
| | | | | either | FL; Down in |
| | | | | DLBCL or | Transformed |
| | | | | FL; | DLBCL |
| | | | | Expression in | |
| | | | | FL to DLBCL | |
| | | | | transformation | |
| Survival; | NM_002046 | GAPD | glyceraldehyde-3- | Genes | Down in |
| DLBCL Vs. | | | phosphate | Correlated | Cured; Up in |
| FL | | | dehydrogenase | with Survival; | Fatal; Up in |
| | | | | Expression in | Transformed |
| | | | | FL to DLBCL | DLBCL |
| | | | | transformation | |
| ABC VS | NM_002648 | PIM1 | pim-1 oncogene | Wright | Low in GC |
| GC | | | | predictor; | DLBCL; Up in |
| DLBCL; | | | | Expression in | Transformation |
| DLBCL Vs. | | | | FL to DLBCL | |
| FL | | | | transformation | |
| DLBCL Vs. | NM_002162 | ICAM3 | intercellular adhesion | Expression in | Down in |
| FL; DLBCL | | | molecule 3 | FL to DLBCL | Transformed |
| vs. MCL | | | | transformation; | DLBCL; |
| | | | | Strong | Down in |
| | | | | Classifier | DLBCL; Up in |
| | | | | Separating | MCL |
| | | | | MCLs and | |
| | | | | DLBCLs | |
| DLBCL Vs. | NM_000598 | IGFBP3 | insulin-like growth | Expression in | Down in |
| FL | | | factor binding protein 3 | FL to DLBCL | Transformed |
| | | | | transformation | DLBCL |
| DLBCL Vs. | NM_000034 | ALDOA | aldolase A, fructose- | Genes | Up in DLBCL; |
| FL | | | bisphosphate | specific to | Down in FL; |
| | | | | either | Up in |
| | | | | DLBCL or | Transformation |
| | | | | FL; | |
| | | | | Expression in | |
| | | | | FL to DLBCL | |
| | | | | transformation | |
| DLBCL Vs. | NM_005998 | CCT3 | chaperonin containing | Genes | Up in DLBCL; |
| FL | | | TCP1, subunit 3 | specific to | Down in FL; |
| | | | (gamma) | either | Up in |
| | | | | DLBCL or | Transformation |
| | | | | FL; | |
| | | | | Expression in | |
| | | | | FL to DLBCL | |
| | | | | transformation | |
| FL vs MCL | NM_024728 | C7orf10 | chromosome 7 open | | up in FL vs |
| | | | reading frame 10 | | MCL (4.5) |
| CLL/SLL vs | BC046632 | FLJ21195 | protein related to DAN | | up in FL vs |
| FL | | | and cerberus | | SLL (4.1) |
| DLBCL Vs. | NM_004619 | TRAF5 | TNF receptor-associated | Genes | Down in |
| FL; MLBCL | | | factor 5 | specific to | DLBCL, Up in |
| vs. DLBCL | | | | either | FL; Low in |
| | | | | DLBCL or | MLBCL |
| | | | | FL | |
| Survival; | NM_021603 | FXYD2 | FXYD domain | Genes | Up in cured; |
| DLBCL Vs. | | | containing ion transport | Correlated | Down in fatal; |
| FL | | | regulator 2 | with Survival; | Up in |
| | | | | Expression in | Transformed |
| | | | | FL to DLBCL | DLBCL |
| | | | | transformation | |
| DLBCL Vs. | NM_001901 | CTGF | connective tissue growth | Expression in | Down in |
| FL | | | factor | FL to DLBCL | Transformed |
| | | | | transformation | DLBCL |
| FL | NM_024713 | FLJ22557 | hypothetical protein | | up in FL (6.5) |
| | | | FLJ22557 | | |
| DLBCL Vs. | NM_004907 | ETR101 | immediate early protein | Expression in | Down in |
| FL | | | | FL to DLBCL | Transformed |
| | | | | transformation | DLBCL |
| DLBCL Vs. | NM_003070 | SMARCA2 | SWI/SNF related, matrix | Expression in | Down in |
| FL | | | associated, actin | FL to DLBCL | Transformed |
| | | | dependent regulator of | transformation | DLBCL |
| | | | chromatin, subfamily a, | | |
| | | | member 2 | | |
| DLBCL Vs. | NM_012203 | GRHPR | glyoxylate | Expression in | Up in |
| FL | | | reductase/hydroxypyruvate | FL to DLBCL | Transformation |
| | | | reductase | transformation | |
| CLL/SLL vs | NM_014686 | KIAA0355 | KIAA0355 | | down in FL vs |
| FL | | | | | SLL (−4.5) |
| FL | AB029034 | PHF8 | PHD finger protein 8 | | down in FL (−10.8) |
| FL | BC001077 | LOC87769 | hypothetical protein | | down in FL (−4.9) |
| | | | BC004360 | | |
| FL | NM_024319 | C1orf35 | chromosome 1 open | | down in FL (−4.4); |
| | | | reading frame 35 | | down in |
| | | | | | FL vs SLL (−5) |
| FL vs MCL | NM_015670 | SENP3 | SUMO1/sentrin/SMT3 | | up in FL vs |
| | | | specific protease 3 | | MCL |

TABLE 8
An HCP set specific for HL, according to one embodiment of the invention

| Feature | Accession | Symbol | Name | Notes | Level |
|---|---|---|---|---|---|
| Survival | NM_001618 | ADPRT | ADP-ribosyltransferase | Genes with | Down in Fatal; |
| (HL) | | | (NAD+; poly (ADP- | prognostic | Up in Cured |
| | | | ribose) polymerase) | value | |
| Survival | NM_000038 | APC | adenomatosis polyposis | Genes with | Down in Fatal; |
| (HL) | | | coli | prognostic | Up in Cured |
| | | | | value | |
| Survival | NM_001226 | CASP6 | caspase 6, apoptosis- | Genes with | Down in Fatal; |
| (HL) | | | related cysteine protease | prognostic | Up in Cured |
| | | | | value | |
| Survival | NM_001795 | CDH5 | cadherin 5, type 2, VE- | Genes with | Up in Fatal; |
| (HL) | | | cadherin (vascular | prognostic | Down in Cured |
| | | | epithelium) | value | |
| Survival | NM_058197 | CDKN2A | cyclin-dependent kinase | Genes with | Down in Fatal; |
| (HL) | | | inhibitor 2A (melanoma, | prognostic | Up in Cured |
| | | | p16, inhibits CDK4) | value | |
| Survival | NM_005215 | DCC | deleted in colorectal | Genes with | Down in Fatal; |
| (HL) | | | carcinoma | prognostic | Up in Cured |
| | | | | value | |
| Survival | NM_004119 | FLT3 | fms-related tyrosine | Genes with | Down in Fatal; |
| (HL) | | | kinase 3 | prognostic | Up in Cured |
| | | | | value | |
| HL vs. | NM_002086 | GRB2 | growth factor receptor- | Differentially | Up only in HL |
| ALCL | | | bound protein 2 | regulated | |
| | | | | between | |
| | | | | ALCL and HL | |
| HL vs. | NM_176783 | PSME1 | proteasome (prosome, | Differentially | Up only in |
| ALCL | | | macropain) activator | regulated | ALCL |
| | | | subunit 1 (PA28 | between | |
| | | | aHCPha) | ALCL and HL | |
| HL vs. | NM_002961 | S100A4 | S100 calcium binding | Differentially | Up only in HL |
| ALCL | | | protein A4 (calcium | regulated | |
| | | | protein, calvasculin, | between | |
| | | | metastasin, murine | ALCL and HL | |
| | | | placental homolog) | | |
| HL | NM_000698 | ALOX5 | arachidonate 5- | Genes unique | Down in HL |
| Signature | | | lipoxygenase | to | |
| | | | | Reed/Sternberg | |
| | | | | cells | |
| HL | NM_001715 | BLK | B lymphoid tyrosine | Genes unique | Down in HL |
| Signature | | | kinase | to | |
| | | | | Reed/Sternberg | |
| | | | | cells | |
| HL | NM_002987 | CCL17 | chemokine (C-C motif) | Genes unique | Up in HL |
| Signature | | | ligand 17 | to | |
| | | | | Reed/Sternberg | |
| | | | | cells | |
| HL | NM_001783 | CD79A | CD79A antigen | Genes unique | Down in HL |
| Signature | | | (immunoglobulin- | to | |
| | | | associated aHCPha) | Reed/Sternberg | |
| | | | | cells | |
| HL | NM_000626 | CD79B | CD79B antigen | Genes unique | Down in HL |
| Signature | | | (immunoglobulin- | to | |
| | | | associated beta) | Reed/Sternberg | |
| | | | | cells | |
| HL | NM_182908 | DHRS2 | dehydrogenase/reductase | Genes unique | Up in HL |
| Signature | | | (SDR family) member 2 | to | |
| | | | | Reed/Sternberg | |
| | | | | cells | |
| HL | NM_004434 | EML1 | echinoderm microtubule | Genes unique | Up in HL |
| Signature | | | associated protein like 1 | to | |
| | | | | Reed/Sternberg | |
| | | | | cells | |
| HL | NM_006495 | EVI2B | ecotropic viral | Genes unique | Down in HL |
| Signature | | | integration site 2B | to | |
| | | | | Reed/Sternberg | |
| | | | | cells | |
| HL | NM_013451 | FER1L3 | fer-1-like 3, myoferlin | Genes unique | Up in HL |
| Signature | | | (C. elegans) | to | |
| | | | | Reed/Sternberg | |
| | | | | cells | |
| HL | NM_002048 | GAS1 | growth arrest-specific 1 | Genes unique | Up in HL |
| Signature | | | | to | |
| | | | | Reed/Sternberg | |
| | | | | cells | |
| HL | BM722299 | H2AFB | H2A histone family, | Genes unique | Up in HL |
| Signature | | | member B | to | |
| | | | | Reed/Sternberg | |
| | | | | cells | |
| HL | NM_002167 | ID3 | inhibitor of DNA | Genes unique | Down in HL |
| Signature | | | binding 3, dominant | to | |
| | | | negative helix-loop-helix | Reed/Sternberg | |
| | | | protein | cells | |
| HL | BC011857 | IGHM | Immunoglobulin heavy | Genes unique | Down in HL |
| Signature | | | constant mu | to | |
| | | | | Reed/Sternberg | |
| | | | | cells | |
| HL | NM_005933 | MLL | myeloid/lymphoid or | Genes unique | Down in HL |
| Signature | | | mixed-lineage leukemia | to | |
| | | | (trithorax homolog, | Reed/Sternberg | |
| | | | Drosophila) | cells | |
| HL | NM_152866 | MS4A1 | membrane-spanning 4- | Genes unique | Down in HL |
| Signature | | | domains, subfamily A, | to | |
| | | | member 1 | Reed/Sternberg | |
| | | | | cells | |
| HL | AB033114 | MTSG1 | mitochondrial tumor | Genes unique | Up in HL |
| Signature | | | suppressor gene 1 | to | |
| | | | | Reed/Sternberg | |
| | | | | cells | |
| HL | NM_004289 | NFE2L3 | nuclear factor (erythroid- | Genes unique | Up in HL |
| Signature | | | derived 2)-like 3 | to | |
| | | | | Reed/Sternberg | |
| | | | | cells | |
| HL | NM_020992 | PDLIM1 | PDZ and LIM domain 1 | Genes unique | Down in HL |
| Signature | | | (elfin) | to | |
| | | | | Reed/Sternberg | |
| | | | | cells | |
| HL | NM_002661 | PLCG2 | phospholipase C, gamma | Genes unique | Down in HL |
| Signature | | | 2 (phosphatidylinositol- | to | |
| | | | specific) | Reed/Sternberg | |
| | | | | cells | |
| HL | NM_006115 | PRAME | preferentially expressed | Genes unique | Up in HL |
| Signature | | | antigen in melanoma | to | |
| | | | | Reed/Sternberg | |
| | | | | cells | |
| HL | NM_002838 | PTPRC | protein tyrosine | Genes unique | Down in HL |
| Signature | | | phosphatase, receptor | to | |
| | | | type, C | Reed/Sternberg | |
| | | | | cells | |
| HL | NM_015055 | SWAP70 | SWAP-70 protein | Genes unique | Down in HL |
| Signature | | | | to | |
| | | | | Reed/Sternberg | |
| | | | | cells | |
| HL | NM_004665 | VNN2 | vanin 2 | Genes unique | Down in HL |
| Signature | | | | to | |
| | | | | Reed/Sternberg | |
| | | | | cells | |
| Signature; | NM_003254 | TIMP1 | tissue inhibitor of | Genes unique | Disagreement: Up |
| HL vs. | | | metalloproteinase 1 | to | in HL; Up |
| ALCL; | | | (erythroid potentiating | Reed/Sternberg | only in ALCL; |
| Survival | | | activity, collagenase | cells; | Up in Fatal; |
| | | | inhibitor) | Differentially | Down in Cured |
| | | | | regulated | |
| | | | | between | |
| | | | | ALCL and | |
| | | | | HL; Genes | |
| | | | | with | |
| | | | | prognostic | |
| | | | | value | |
| Survival | NM_024408 | NOTCH2 | Notch homolog 2 | Genes with | Down in Fatal; |
| (HL) | | | (Drosophila) | prognostic | Up in Cured |
| | | | | value | |
| Survival | NM_000314 | PTEN | phosphatase and tensin | Genes with | Down in Fatal; |
| (HL) | | | homolog (mutated in | prognostic | Up in Cured |
| | | | multiple advanced | value | |
| | | | cancers 1) | | |
| Survival | NM_003005 | SEHCP | selectin P (granule | Genes with | Up in Fatal; |
| (HL) | | | membrane protein | prognostic | Down in Cured |
| | | | 140 kDa, antigen CD62) | value | |
| Survival | NM_003810 | TNFSF10 | tumor necrosis factor | Genes with | Down in Fatal; |
| (HL) | | | (ligand) superfamily, | prognostic | Up in Cured |
| | | | member 10 | value | |
| HL vs. | NM_005178 | BCL3 | B-cell CLL/lymphoma 3 | Diagnostic | Up only in |
| ALCL | | | | Marker | ALCL |
| HL vs. | NM_000485 | APRT | adenine | Differentially | Up in Both HL |
| ALCL | | | phosphoribosyltransferase | regulated | and ALCL |
| | | | | between | |
| | | | | ALCL and HL | |
| HL vs. | NM_001728 | BSG | basigin (OK blood | Differentially | Up only in |
| ALCL | | | group) | regulated | ALCL |
| | | | | between | |
| | | | | ALCL and HL | |
| HL vs. | NM_006763 | BTG2 | BTG family, member 2 | Differentially | Up in Both HL |
| ALCL | | | | regulated | and ALCL |
| | | | | between | |
| | | | | ALCL and HL | |
| HL vs. | NM_001743 | CALM2 | calmodulin 2 | Differentially | Up in Both HL |
| ALCL | | | (phosphorylase kinase, | regulated | and ALCL |
| | | | delta) | between | |
| | | | | ALCL and HL | |
| HL vs. | NM_033306 | CASP4 | caspase 4, apoptosis- | Differentially | Up only in |
| ALCL | | | related cysteine protease | regulated | ALCL |
| | | | | between | |
| | | | | ALCL and HL | |
| HL vs. | NM_005340 | HINT1 | histidine triad nucleotide | Differentially | Up only in |
| ALCL | | | binding protein 1 | regulated | ALCL |
| | | | | between | |
| | | | | ALCL and HL | |
| HL vs. | NM_002228 | JUN | v-jun sarcoma virus 17 | Differentially | Up only in HL |
| ALCL | | | oncogene homolog | regulated | |
| | | | (avian) | between | |
| | | | | ALCL and HL | |
| HL vs. | NM_006191 | PA2G4 | proliferation-associated | Differentially | Up in Both HL |
| ALCL | | | 2G4, 38 kDa | regulated | and ALCL |
| | | | | between | |
| | | | | ALCL and HL | |
| HL vs. | NM_030666 | SERPINB1 | serine (or cysteine) | Differentially | Up in Both HL |
| ALCL | | | proteinase inhibitor, | regulated | and ALCL |
| | | | clade B (ovalbumin), | between | |
| | | | member 1 | ALCL and HL | |
| HL vs. | NM_003955 | SOCS3 | suppressor of cytokine | Differentially | Up only in |
| ALCL | | | signaling 3 | regulated | ALCL |
| | | | | between | |
| | | | | ALCL and HL | |
| HL vs. | NM_003254 | TIMP1 | tissue inhibitor of | Genes unique | Disagreement: Up |
| ALCL | | | metalloproteinase 1 | to | in HL; Up |
| | | | (erythroid potentiating | Reed/Sternberg | only in ALCL; |
| | | | activity, collagenase | cells; | Up in Fatal; |
| | | | inhibitor) | Differentially | Down in Cured |
| | | | | regulated | |
| | | | | between | |
| | | | | ALCL and | |
| | | | | HL; Genes | |
| | | | | with | |
| | | | | prognostic | |
| | | | | value | |

TABLE 9
An HCP set specific for MCL, according to one embodiment of the invention

| Feature | Accession | Symbol | Name | Notes | Level |
|---|---|---|---|---|---|
| MCL vs. | NM_001675 | ATF4 | activating transcription | High in | Up in MCL- |
| MCL-BV | | | factor 4 (tax-responsive | Aggressive | BV vs. MCL |
| | | | enhancer element B67) | Blast Variant | |
| MCL vs. | NM_001826 | CKS1B | CDC28 protein kinase | High in | Up in MCL- |
| MCL-BV | | | regulatory subunit 1B | Aggressive | BV vs. MCL |
| | | | | Blast Variant; | |
| | | | | RT-Confirmed | |
| MCL vs. | NM_001831 | CLU | clusterin (complement | RT-Confirmed | Up in MCL- |
| MCL-BV | | | lysis inhibitor, SP- | | BV vs. MCL |
| | | | 40,40, sulfated | | |
| | | | glycoprotein 2, | | |
| | | | testosterone-repressed | | |
| | | | prostate message 2, | | |
| | | | apolipoprotein J) | | |
| MCL vs. | NM_001344 | DAD1 | defender against cell | RT-Confirmed | Up in MCL- |
| MCL-BV | | | death 1 | | BV vs. MCL |
| MCL vs. | NM_005243 | EWSR1 | Ewing sarcoma | RT-Confirmed | Up in MCL- |
| MCL-BV | | | breakpoint region 1 | | BV vs. MCL |
| MCL vs. | NM_006769 | LMO4 | LIM domain only 4 | High in | Up in MCL- |
| MCL-BV | | | | Aggressive | BV vs. MCL |
| | | | | Blast Variant; | |
| | | | | RT-Confirmed | |
| MCL vs. | NM_005582 | LY64 | lymphocyte antigen 64 | High in | Up in MCL- |
| MCL-BV | | | homolog, | Aggressive | BV vs. MCL |
| | | | radioprotective 105 kDa | Blast Variant; | |
| | | | (mouse) | RT-Confirmed | |
| MCL vs. | NM_004689 | MTA1 | metastasis associated 1 | RT-Confirmed | Up in MCL- |
| MCL-BV | | | | | BV vs. MCL |
| MCL vs. | NM_002466 | MYBL2 | v-myb myeloblastosis | RT-Confirmed | Up in MCL- |
| MCL-BV | | | viral oncogene | | BV vs. MCL |
| | | | homolog (avian)-like 2 | | |
| MCL vs. | NM_002648 | PIM1 | pim-1 oncogene | High in | Up in MCL- |
| MCL-BV | | | | Aggressive | BV vs. MCL |
| | | | | Blast Variant; | |
| | | | | RT-Confirmed | |
| MCL vs. | NM_003403 | YY1 | YY1 transcription | High in | Up in MCL- |
| MCL-BV | | | factor | Aggressive | BV vs. MCL |
| | | | | Blast Variant; | |
| | | | | RT-Confirmed | |
| MCL | NM_053056 | CCND1 | cyclin D1 (PRAD1: | RT-PCR | Up in MCL; |
| Signature | | | parathyroid | Comparison of | Down in |
| | | | adenomatosis 1) | CLL and MCL | DLBCL; Up |
| | | | | | in MCL; up |
| | | | | | in MCL |
| MCL | NM_000633 | BCL2 | B-cell CLL/lymphoma 2 | Gene | High in MCL |
| Signature | | | | Expression | and DLBCL |
| | | | | Analysis in | cell lines; |
| | | | | MCL cell lines; | Down in |
| | | | | RT-PCR | MCL & |
| | | | | Comparison of | MZL, Up in |
| | | | | CLL, MCL & | CLL |
| | | | | MZL | |
| MCL | NM_002162 | ICAM3 | intercellular adhesion | Strong | Down in |
| Signature | | | molecule 3 | Classifier | DLBCL; Up |
| | | | | Separating | in MCL |
| | | | | MCLs and | |
| | | | | DLBCLs | |
| Survival; | NM_002592 | PCNA | proliferating cell | Proliferation | High in Short |
| MCL vs. | | | nuclear antigen | Signature | Survival; Up |
| MCL-BV | | | | | in MCL-BV |
| | | | | | vs. MCL |
| MCL | NM_000698 | ALOX5 | arachidonate 5- | | up in MCL |
| | | | lipoxygenase | | (5.8) |
| MCL vs. | NM_005104 | BRD2 | bromodomain | High in | Up in MCL- |
| MCL-BV | | | containing 2 | Aggressive | BV vs. MCL |
| | | | | Blast Variant | |
| MCL vs. | NM_001728 | BSG | basigin (OK blood | High in | Up in MCL- |
| MCL-BV | | | group) | Aggressive | BV vs. MCL |
| | | | | Blast Variant | |
| MCL vs. | NM_001469 | G22P1 | thyroid autoantigen | High in | Up in MCL- |
| MCL-BV | | | 70 kDa (Ku antigen) | Aggressive | BV vs. MCL |
| | | | | Blast Variant | |
| MCL vs. | NM_031844 | HNRPU | heterogeneous nuclear | High in | Up in MCL- |
| MCL-BV | | | ribonucleoprotein U | Aggressive | BV vs. MCL |
| | | | (scaffold attachment | Blast Variant | |
| | | | factor A) | | |
| MCL vs. | NM_002460 | IRF4 | interferon regulatory | High in | Up in MCL- |
| MCL-BV | | | factor 4 | Aggressive | BV vs. MCL |
| | | | | Blast Variant | |
| MCL vs. | NM_006152 | LRMP | lymphoid-restricted | High in | Up in MCL- |
| MCL-BV | | | membrane protein | Aggressive | BV vs. MCL |
| | | | | Blast Variant | |
| MCL vs. | NM_006819 | STIP1 | stress-induced- | High in | Up in MCL- |
| MCL-BV | | | phosphoprotein 1 | Aggressive | BV vs. MCL |
| | | | (Hsp70/Hsp90- | Blast Variant | |
| | | | organizing protein) | | |
| MCL | NM_001092 | ABR | active BCR-related | Signature genes | Down in |
| Signature | | | gene | common to both | DLBCL; Up |
| | | | | Shipp and | in MCL |
| | | | | Alizadeh; | |
| | | | | Strong | |
| | | | | Classifier | |
| | | | | Separating | |
| | | | | MCLs and | |
| | | | | DLBCLs | |
| MCL | NM_156039 | CSF3R | colony stimulating | Strong | Down in |
| Signature | | | factor 3 receptor | Classifier | DLBCL; Up |
| | | | (granulocyte) | Separating | in MCL |
| | | | | MCLs and | |
| | | | | DLBCLs | |
| MCL | NM_004941 | DHX8 | DEAH (Asp-Glu-Ala- | Strong | Down in |
| Signature | | | His) box polypeptide 8 | Classifier | DLBCL; Up |
| | | | | Separating | in MCL |
| | | | | MCLs and | |
| | | | | DLBCLs | |
| MCL | BC025340 | MGC39372 | hypothetical protein | | up in MCL |
| | | | MGC39372 | | (8.4); down |
| | | | | | in FL vs |
| | | | | | MCL (−13.6); |
| | | | | | up in MCL vs |
| | | | | | SLL (6.3) |
| MCL | NM_004513 | IL16 | interleukin 16 | | up in FL vs |
| | | | (lymphocyte | | MCL (4.9); |
| | | | chemoattractant factor) | | down in |
| | | | | | MCL vs SLL |
| | | | | | (−7.8) |
| MCL vs | NM_002466 | MYBL2 | v-myb myeloblastosis | | up in MCL vs |
| CLL/SLL | | | viral oncogene | | SLL (5.1) |
| | | | homolog (avian)-like 2 | | |
| Signature; | NM_000075 | CDK4 | cyclin-dependent | RT-PCR | Up in MCL; |
| MCL vs. | | | kinase 4 | Comparison of | Up in MCL- |
| MCL-BV | | | | CLL and MCL; | BV vs. MCL |
| | | | | RT confirmed | |
| CLL/SLL | AB018263 | KIAA0720 | putative NFkB | | Up in MCL |
| vs MCL | | | activating protein | | vs SLL (4.3) |
| FL; MCL | NM_000424 | KRT5 | keratin 5 | | down in FL (−5.3); |
| | | | (epidermolysis bullosa | | down in |
| | | | simplex, Dowling- | | MCL (−4) |
| | | | Meara/Kobner/Weber- | | |
| | | | Cockayne types) | | |
| FL vs | AB037771 | USP53 | ubiquitin specific | | up in FL vs |
| MCL | | | protease 53 | | MCL (4.2) |
| MCL vs | NM_032873 | KIAA1959 | nm23-phosphorylated | | down in |
| CLL/SLL | | | unknown substrate | | MCL vs SLL |
| | | | | | (−5.3) |
| FL vs | NM_000418 | IL4R | interleukin 4 receptor | | up in FL vs |
| MCL | | | | | MCL (5.4) |
| MCL vs | NM_000418 | IL4R | interleukin 4 receptor | | down in |
| CLL/SLL | | | | | MCL vs SLL |
| | | | | | (−7.1) |
| MCL vs | NM_023037 | 13CDNA73 | hypothetical protein | | MCL vs SLL |
| CLL/SLL | | | CG003 | | (−7.6) |
| MCL | AF509494 | KIAA1407 | KIAA1407 protein | | up in MCL |
| | | | | | (10) |
| FL vs | NM_001781 | CD69 | CD69 antigen (p60, | | down in FL |
| MCL | | | early T-cell activation | | vs MCL (−4.1) |
| | | | antigen) | | |
| MCL vs | NM_030926 | ITM2C | integral membrane | | up in MCL vs |
| SLL | | | protein 2C | | SLL |
| MCL | AF331856 | DNMT3A | DNA (cytosine-5-)- | | down in FL |
| | | | methyltransferase 3 | | vs MCL (−8); |
| | | | aHCPha | | up in MCL vs |
| | | | | | SLL (5.2) |
| FL vs | NM_000405 | GM2A | GM2 ganglioside | | down in FL |
| MCL | | | activator | | vs MCL (−4.4) |
| CLL/SLL | NM_000483 | APOC2 | apolipoprotein C-II | | up in MCL vs |
| vs MCL | | | | | SLL (5.6) |
| FL vs | NM_002228 | JUN | v-jun sarcoma virus 17 | | down in FL |
| MCL | | | oncogene homolog | | vs MCL (−4.6) |
| | | | (avian) | | |
| FL vs | NM_006889 | CD86 | CD86 antigen (CD28 | | up in FL vs |
| MCL | | | antigen ligand 2, B7-2 | | MCL (7.9) |
| | | | antigen) | | |
| CLL/SLL | NM_003480 | MFAP5 | microfibrillar | | up in MCL vs |
| vs MCL | | | associated protein 5 | | CLL/SLL |
| | | | | | (4.2) |
| MCL | NM_181430 | ILF1 | interleukin enhancer | Strong | Down in |
| Signature | | | binding factor 1 | Classifier | DLBCL; Up |
| | | | | Separating | in MCL |
| | | | | MCLs and | |
| | | | | DLBCLs | |
| MCL | NM_003222 | TFAP2C | transcription factor AP- | | down in |
| | | | 2 gamma (activating | | MCL (−8.2) |
| | | | enhancer binding | | |
| | | | protein 2 gamma) | | |
| FL vs | NM_006164 | NFE2L2 | nuclear factor | | up in FL vs |
| MCL | | | (erythroid-derived 2)- | | MCL (4.1) |
| | | | like 2 | | |
| MCL | NM_004235 | KLF4 | Kruppel-like factor 4 | | up in MCL |
| | | | (gut) | | (5) |
| MCL vs | NM_003651 | CSDA | cold shock domain | | down in |
| CLL/SLL | | | protein A | | MCL vs SLL |
| | | | | | (−4.4) |
| MCL | NM_001888 | CRYM | crystallin, mu | | up in MCL |
| | | | | | (8.5); down |
| | | | | | in FL vs |
| | | | | | MCL (−7.3); |
| | | | | | up in MCL vs |
| | | | | | SLL (9.3) |
| MCL | NM_001675 | ATF4 | activating transcription | | up in MCL |
| | | | factor 4 (tax-responsive | | (4.3) |
| | | | enhancer element B67) | | |
| MCL | NM_003804 | RIPK1 | receptor (TNFRSF)- | | down in |
| | | | interacting serine- | | MCL (−4.2) |
| | | | threonine kinase 1 | | |
| MCL | NM_004924 | ACTN4 | actinin, aHCPha 4 | | up in MCL |
| | | | | | (4.5) |
| FL vs | NM_018842 | LOC55971 | insulin receptor | | down in FL |
| MCL | | | tyrosine kinase | | vs MCL (−8.4) |
| | | | substrate | | |
| MCL | NM_002483 | CEACAM6 | carcinoembryonic | | down in |
| | | | antigen-related cell | | MCL (−4.4); |
| | | | adhesion molecule 6 | | up in FL vs |
| | | | (non-specific cross | | MCL (4.7) |
| | | | reacting antigen) | | |
| FL vs | NM_001640 | APEH | N-acylaminoacyl- | | down in |
| MCL | | | peptide hydrolase | | MCL (−6.5); |
| | | | | | up in FL vs |
| | | | | | MCL (6) |
| FL vs | NM_006475 | POSTN | periostin, osteoblast | | Up in FL vs |
| MCL | | | specific factor | | MCL (4) |
| MCL | NM_000700 | ANXA1 | annexin A1 | | down in |
| | | | | | MCL (−4.1) |
| FL vs | NM_021813 | BACH2 | BTB and CNC | | up in FL vs |
| MCL | | | homology 1, basic | | MCL (5.5) |
| | | | leucine zipper | | |
| | | | transcription factor 2 | | |
| DLBCL | NM_053056 | CCND1 | cyclin D1 (PRAD1: | Strong | Down in |
| vs. MCL | | | parathyroid | Classifier | DLBCL; Up |
| | | | adenomatosis 1) | Separating | in MCL |
| | | | | MCLs and | |
| | | | | DLBCLs | |
| CLL vs. | NM_002467 | MYC | v-myc | RT-PCR | High in CLL |
| MCL | | | myelocytomatosis viral | analysis shows | |
| | | | oncogene homolog | up regulation in | |
| | | | (avian) | CLL | |
| CLL/SLL | NM_002933 | RNASE1 | ribonuclease, RNase A | | down in |
| vs MCL | | | family, 1 (pancreatic) | | MCL vs SLL |
| | | | | | (−5.4) |
| MCL | NM_022829 | SLC13A3 | solute carrier family 13 | | up in MCL |
| | | | (sodium-dependent | | (4.9) |
| | | | dicarboxylate | | |
| | | | transporter), member 3 | | |
| MCL | NM_003339 | UBE2D2 | ubiquitin-conjugating | | up in MCL |
| | | | enzyme E2D 2 | | (4.4) |
| | | | (UBC4/5 homolog, | | |
| | | | yeast) | | |
| ABC VS | NM_001092 | ABR | active BCR-related | Signature genes | Uncertain in |
| GC | | | gene | common to both | Common |
| DLBCL; | | | | Shipp and | Genes; Down |
| DLBCL | | | | Alizadeh; | in DLBCL; |
| vs. MCL | | | | Strong | Up in MCL |
| | | | | Classifier | |
| | | | | Separating | |
| | | | | MCLs and | |
| | | | | DLBCLs | |
| DBFCL | NM_002162 | ICAM3 | intercellular adhesion | Expression in | Down in |
| Vs. FL; | | | molecule 3 | FL to DLBCL | Transformed |
| DLBCL | | | | transformation; | DLBCL; |
| vs. MCL | | | | Strong | Down in |
| | | | | Classifier | DLBCL; Up |
| | | | | Separating | in MCL |
| | | | | MCLs and | |
| | | | | DLBCLs | |
| FL vs | NM_024728 | C7orf10 | chromosome 7 open | | up in FL vs |
| MCL | | | reading frame 10 | | MCL (4.5) |
| MCL | NM_025263 | PRR3 | proline rich 3 | | up in MCL |
| | | | | | (4.3) |
| DLBCL | NM_019841 | TRPV5 | transient receptor | Strong | Down in |
| vs. MCL; | | | potential cation | Classifier | DLBCL; Up |
| CD5+/− | | | channel, subfamily V, | Separating | in MCL; |
| DLBCL | | | member 5 | MCLs and | Down in |
| | | | | DLBCLs and | CD5+; Up in |
| | | | | CD5+ from | CD5− |
| | | | | CD5− DLBCLs | |
| MCL | NM_024713 | FLJ22557 | hypothetical protein | | up in MCL |
| | | | FLJ22557 | | (15.7) |
| MCL | AK092000 | SLC3A1 | solute carrier family 3 | | up in MCL |
| | | | (cystine, dibasic and | | (4.3) |
| | | | neutral amino acid | | |
| | | | transporters, activator | | |
| | | | of cystine, dibasic and | | |
| | | | neutral amino acid | | |
| | | | transport), member 1 | | |
| MCL | NM_022436 | ABCG5 | ATP-binding cassette, | | down in |
| | | | sub-family G | | MCL (−4.1) |
| | | | (WHITE), member 5 | | |
| | | | (sterolin 1) | | |
| MCL | NM_004475 | FLOT2 | flotilline 2 | Strong | Down in |
| Signature | | | | Classifier | DLBCL; Up |
| | | | | Separating | in MCL |
| | | | | MCLs and | |
| | | | | DLBCLs | |
| DLBCL | NM_134269 | SMTN | smoothelin | Strong | Down in |
| vs. MCL | | | | Classifier | DLBCL; Up |
| | | | | Separating | in MCL |
| | | | | MCLs and | |
| | | | | DLBCLs | |
| CLL vs. | NM_000075 | CDK4 | cyclin-dependent | MCL, CLL | High in CLL |
| MCL | | | kinase 4 | Signature&CLL | |
| | | | | Vs. MCL | |
| | | | | Distinguisher | |
| DLBCL | NM_004941 | DHX8 | DEAH (Asp-Glu-Ala- | Strong | Down in |
| vs. MCL | | | His) box polypeptide 8 | Classifier | DLBCL; Up |
| | | | | Separating | in MCL |
| | | | | MCLs and | |
| | | | | DLBCLs | |
| DLBCL | NM_004475 | FLOT2 | flotillin 2 | Strong | Down in |
| vs. MCL | | | | Classifier | DLBCL; Up |
| | | | | Separating | in MCL |
| | | | | MCLs and | |
| | | | | DLBCLs | |
| DLBCL | NM_181430 | ILF1 | interleukin enhancer | Strong | Down in |
| vs. MCL | | | binding factor 1 | Classifier | DLBCL; Up |
| | | | | Separating | in MCL |
| | | | | MCLs and | |
| | | | | DLBCLs | |
| DLBCL | NM_156039 | CSF3R | colony stimulating | Strong | Down in |
| vs. MCL | | | factor 3 receptor | Classifier | DLBCL; Up |
| | | | (granulocyte) | Separating | in MCL |
| | | | | MCLs and | |
| | | | | DLBCLs | |
| DLBCL | NM_000277 | PAH | phenylalanine | Strong | Down in |
| vs. MCL | | | hydroxylase | Classifier | DLBCL; Up |
| | | | | Separating | in MCL |
| | | | | MCLs and | |
| | | | | DLBCLs | |
| MCL | BC022095 | C6orf110 | chromosome 6 open | | up in MCL |
| | | | reading frame 110 | | (4.8) |
| MCL | NM_031305 | ARHGAP24 | Rho GTPase activating | | up in MCL |
| | | | protein 24 | | (5.6) |
| MCL | AB029034 | PHF8 | PHD finger protein 8 | | down in |
| | | | | | MCL (−5.8) |
| MCL | NM_198243 | ASB7 | ankyrin repeat and | | down in |
| | | | SOCS box-containing 7 | | MCL (−6) |
| MCL | NM_016570 | PTX1 | PTX1 protein | | up in FL vs |
| | | | | | MCL (5.8); |
| | | | | | down in |
| | | | | | MCL vs SLL |
| | | | | | (−8.8) |
| MCL | BC001077 | LOC87769 | hypothetical protein | | down in |
| | | | BC004360 | | MCL (−4.9) |
| MCL | NM_138379 | LOC91937 | hypothetical protein | | down in |
| | | | BC008988 | | MCL (−4.1); |
| | | | | | down in |
| | | | | | MCL vs SLL |
| | | | | | (−4.3) |
| FL vs | NM_015670 | SENP3 | SUMO1/sentrin/SMT3 | | up in FL vs |
| MCL | | | specific protease 3 | | MCL |
| MCL | BX647445 | GTF2H2 | general transcription | | up in MCL |
| | | | factor IIH, polypeptide | | (5.9) |
| | | | 2, 44 kDa | | |
| MCL | NM_030956 | TLR10 | toll-like receptor 10 | Molecular | Up in MCL |
| Signature | | | | Diagnosis | vs. |
| | | | | | CLL/DLBCL |
| MCL | NM_000277 | PAH | phenylalanine | Strong | Down in |
| Signature | | | hydroxylase | Classifier | DLBCL; Up |
| | | | | Separating | in MCL |
| | | | | MCLs and | |
| | | | | DLBCLs | |
| MCL | NM_134269 | SMTN | smoothelin | Strong | Down in |
| Signature | | | | Classifier | DLBCL; Up |
| | | | | Separating | in MCL |
| | | | | MCLs and | |
| | | | | DLBCLs | |
| MCL | NM_019841 | TRPV5 | transient receptor | Strong | Down in |
| Signature | | | potential cation | Classifier | DLBCL; Up |
| | | | channel, subfamily V, | Separating | in MCL; |
| | | | member 5 | MCLs and | Down in |
| | | | | DLBCLs and | CD5+; Up in |
| | | | | CD5+ from | CD5− |
| | | | | CD5− DLBCLs | |
| MCL | NM_001760 | CCND3 | cyclin D3 | RT-PCR | Up in MCL |
| Signature | | | | Comparison of | vs. CLL |
| | | | | CLL and MCL | |
| MCL | NM_004064 | CDKN1B | cyclin-dependent | RT-PCR | Down in |
| Signature | | | kinase inhibitor 1B | Comparison of | MCL vs. |
| | | | (p27, Kip1) | CLL and MCL | CLL |
| MCL | NM_005225 | E2F1 | E2F transcription | RT-PCR | Up in MCL |
| Signature | | | factor 1 | Comparison of | vs. CLL |
| | | | | CLL and MCL | |
| MCL | NM_003810 | TNFSF10 | tumor necrosis factor | RT-PCR | Up in MCL |
| Signature | | | (ligand) superfamily, | Comparison of | vs. CLL |
| | | | member 10 | CLL and MCL | |
| MCL | NM_013995 | LAMP2 | lysosomal-associated | Gene | High in MCL |
| Signature | | | membrane protein 2 | Expression | and DLBCL |
| | | | | Analysis in | cell lines |
| | | | | MCL cell lines | |
| MCL | NM_000698 | ALOX5 | arachidonate 5- | Molecular | Up in MCL |
| Signature | | | lipoxygenase | Diagnosis | vs. |
| | | | | | CLL/DLBCL |
| MCL | NM_017935 | BANK1 | B-cell scaffold protein | Molecular | Up in MCL |
| Signature | | | with ankyrin repeats 1 | Diagnosis | vs. |
| | | | | | CLL/DLBCL |
| MCL | NM_004126 | GNG11 | guanine nucleotide | Molecular | Up in MCL |
| Signature | | | binding protein (G | Diagnosis | vs. |
| | | | protein), gamma 11 | | CLL/DLBCL |
| MCL | NM_000598 | IGFBP3 | insulin-like growth | Molecular | Up in MCL |
| Signature | | | factor binding protein 3 | Diagnosis | vs. |
| | | | | | CLL/DLBCL |
| MCL | NM_007360 | KLRK1 | killer cell lectin-like | Molecular | Up in MCL |
| Signature | | | receptor subfamily K, | Diagnosis | vs. |
| | | | member 1 | | CLL/DLBCL |
| CLL vs. | NM_004454 | ETV5 | ets variant gene 5 (ets- | RT-PCR | High in CLL |
| MCL | | | related molecule) | analysis shows | |
| | | | | up regulation in | |
| | | | | CLL | |
| MCL | NM_001647 | APOD | apolipoprotein D | | up in MCL |
| | | | | | (5.5); down |
| | | | | | in FL vs |
| | | | | | MCL (−8.3) |
| MCL | NM_015436 | RCHY1 | ring finger and CHY | | up in MCL |
| | | | zinc finger domain | | (4.3) |
| | | | containing 1 | | |
| CLL vs. | NM_001760 | CCND3 | cyclin D3 | CLL Vs. MCL | High in CLL |
| MCL | | | | Distinguisher | |
| MCL | NM_017784 | OSBPL10 | oxysterol binding | | up in MCL |
| | | | protein-like 10 | | (4.7) |
| CLL/SLL | NM_020944 | GBA2 | glucosidase, beta (bile | | down in |
| vs MCL | | | acid) 2 | | MCL vs SLL |
| | | | | | (−5.1) |
| CLL vs. | NM_053056 | CCND1 | cyclin D1 | MCL, CLL | High in CLL |
| MCL | | | (BCL1/PRAD1: | Signature&CLL | |
| | | | parathyroid | Vs. MCL | |
| | | | adenomatosis 1) | Distinguisher | |
| MCL | NM_004225 | MFHAS1 | malignant fibrous | Molecular | Up in MCL |
| Signature | | | histiocytoma amplified | Diagnosis | vs. |
| | | | sequence 1 | | CLL/DLBCL |
| MCL | NM_014257 | CD209L | CD209 antigen-like | | down in |
| | | | | | MCL (−5.1); |
| | | | | | down in |
| | | | | | MCL vs SLL |
| | | | | | (−4.8) |
| MCL | NM_152866 | MS4A1 | membrane-spanning 4- | Molecular | Up in MCL |
| Signature | | | domains, subfamily A, | Diagnosis | vs. |
| | | | member 1 | | CLL/DLBCL |
| MCL | NM_004827 | ABCG2 | ATP-binding cassette, | | down in |
| | | | sub-family G | | MCL (−4.6) |
| | | | (WHITE), member 2 | | |
| Survival | NM_006325 | RAN | RAN, member RAS | Proliferation | High in Short |
| (MCL) | | | oncogene family | Signature | Survival |
| Survival | NM_002129 | HMGB2 | high-mobility group | Proliferation | High in Short |
| (MCL) | | | box 2 | Signature | Survival |
| MCL | NM_003254 | TIMP1 | tissue inhibitor of | RT-PCR | Up in MCL, |
| Signature | | | metalloproteinase 1 | Comparison of | Down in |
| | | | (erythroid potentiating | CLL, MCL & | MZL and |
| | | | activity, collagenase | MZL | CLL |
| | | | inhibitor) | | |
| MCL | NM_024408 | NOTCH2 | Notch homolog 2 | RT-PCR | Down in |
| Signature | | | (Drosophila) | Comparison of | MCL & CLL, |
| | | | | CLL, MCL & | Up in MZL |
| | | | | MZL | |
| CLL vs. | NM_004064 | CDKN1B | cyclin-dependent | MCL, CLL | High in CLL |
| MCL | | | kinase inhibitor 1B | Signature&CLL | |
| | | | (p27, Kip1) | Vs. MCL | |
| | | | | Distinguisher | |
| CLL/SLL | NM_001830 | CLCN4 | chloride channel 4 | | down in |
| vs MCL | | | | | MCL vs SLL |
| | | | | | (−4.9) |

TABLE 10
An HCP set specific for DLBCL, according to one embodiment
of the invention

| DLBCL | |
|---|---|
| Signature | |
| Accession | Symbol |
| NM_000785.2 | CYP27B1 |
| NM_005652.2 | TERF2 |
| NM_006317.3 | BASP1 |
| NM_057735.1 | CCNE2 |
| NM_145804.1 | ABTB2 |
| NM_182776.1 | MCM7 |
| NM_001067.2 | TOP2A |
| NM_002426.1 | MMP12 |
| NM_000075.2 | CDK4 |
| NM_000877.2 | IL1R1 |
| NM_002661.1 | PLCG2 |
| NM_005658.2 | TRAF1 |
| NM_001951.2 | E2F5 |
| NM_002358.2 | MAD2L1 |
| NM_021950.2 | MS4A1 |
| NM_000269.2 | NME1 |
| NM_001826.1 | CKS1B |
| NM_175870.3 | LOC90925 |
| NM_182746.1 | MCM4 |
| NM_004460.2 | FAP |
| NM_006892.3 | DNMT3B |
| NM_012203.1 | GRHPR |
| AI809213.1 | RGS13 |
| NM_001197.3 | BIK |
| NM_001618.2 | PARP1 |
| NM_001771.1 | CD22 |
| NM_002692.2 | POLE2 |
| NM_031942.3 | CDCA7 |
| NM_015641.2 | TES |
| NM_014456.3 | PDCD4 |
| NM_181443.1 | BTBD3 |
| H57732.1 | TGFBR2 |
| NM_004445.1 | EPHB6 |
| NM_003243.1 | TGFBR3 |
| NM_004951.2 | EBI2 |
| NM_005935.1 | MLLT2 |
| NM_000633.1 | BCL2 |
| NM_000927.3 | ABCB1 |
| NM_002350.1 | LYN |
| NM_001558.2 | IL10RA |
| NM_005213.2 | CSTA |
| NM_005127.2 | CLECSF2 |
| W56129.1 | SFRS9 |
| NM_004358.3 | CDC25B |
| NM_004126.2 | GNG11 |
| BX504817.1 | SFRS7 |
| AI597616.1 | MRPL33 |
| NM_006257.2 | PRKCQ |
| NM_000873.2 | ICAM2 |
| NM_002943.2 | RORA |
| NM_001230.3 | CASP10 |

TABLE 11
An HCP set specific for FL, according to one embodiment
of the invention

| FL Signature | |
|---|---|
| Accession | Symbol |
| NM_004619.2 | TRAF5 |
| NM_015187.1 | KIAA0746 |
| NM_006317.3 | BASP1 |
| NM_152785.2 | GCET2 |
| NM_006558.1 | KHDRBS3 |
| NM_017784.3 | OSBPL10 |
| NM_145804.1 | ABTB2 |
| NM_006475.1 | POSTN |
| NM_002002.3 | FCER2 |
| NM_000877.2 | IL1R1 |
| NM_001770.3 | CD19 |
| NM_004356.2 | CD81 |
| NM_005658.2 | TRAF1 |
| NM_006986.2 | MAGED1 |
| NM_001951.2 | E2F5 |
| NM_021950.2 | MS4A1 |
| NM_175870.3 | LOC90925 |
| NM_182746.1 | MCM4 |
| NM_002646.2 | PIK3C2B |
| NM_144646.2 | IGJ |
| NM_001242.3 | TNFRSF7 |
| H53164.1 | IRF8 |
| NM_001877.2 | CR2 |
| NM_005292.2 | GPR18 |
| NM_005214.2 | CTLA4 |
| AI809213.1 | RGS13 |
| AI634809.1 | ARID5B |
| NM_013230.1 | CD24 |
| NM_000237.1 | LPL |
| NM_138379.1 | TIMD4 |
| NM_001197.3 | BIK |
| NM_001831.2 | CLU |
| NM_002101.3 | GYPC |
| NM_001618.2 | PARP1 |
| NM_001771.1 | CD22 |
| NM_016336.2 | UBE2J1 |
| NM_030764.2 | SPAP1 |
| NM_031942.3 | CDCA7 |
| NM_006274.2 | CCL19 |
| NM_016187.1 | BIN2 |
| NM_000271.1 | NPC1 |
| NM_138340.3 | ABHD3 |
| NM_003332.2 | TYROBP |
| NM_006254.3 | PRKCD |
| NM_003243.1 | TGFBR3 |
| NM_002350.1 | LYN |
| NM_000917.1 | P4HA1 |
| NM_005077.3 | TLE1 |
| NM_004184.3 | WARS |
| NM_005601.3 | NKG7 |
| NM_032991.1 | CASP3 |
| NM_003810.2 | TNFSF10 |
| NM_001425.1 | EMP3 |
| NM_000397.2 | CYBB |
| NM_002155.3 | HSPA6 |
| NM_005610.1 | RBBP4 |
| NM_005638.3 | SYBL1 |
| NM_001110.1 | ADAM10 |
| NM_001350.2 | DAXX |
| NM_000295.3 | SERPINA1 |
| BX504817.1 | SFRS7 |
| AI597616.1 | MRPL33 |
| NM_000585.2 | IL15 |
| NM_002305.2 | LGALS1 |
| NM_007311.2 | BZRP |
| NM_006332.3 | IFI30 |
| NM_007360.1 | KLRK1 |
| NM_024708.2 | ASB7 |
| NM_033208.2 | TIGD7 |
| NM_000189.4 | HK2 |

TABLE 12
An HCP set specific for HL, according to one embodiment
of the invention

| HL Signature | |
|---|---|
| Accession | Symbol |
| NM_006274.2 | CCL19 |
| NM_000785.2 | CYP27B1 |
| NM_014762.1 | DHCR24 |
| NM_005606.3 | LGMN |
| NM_003226.2 | TFF3 |
| NM_002426.1 | MMP12 |
| NM_000877.2 | IL1R1 |
| NM_004356.2 | CD81 |
| NM_000483.3 | APOC2 |
| NM_005658.2 | TRAF1 |
| NM_003037.1 | SLAMF1 |
| NM_006115.3 | PRAME |
| NM_000853.1 | GSTT1 |
| NM_002646.2 | PIK3C2B |
| NM_016081.2 | KIAA0992 |
| NM_005214.2 | CTLA4 |
| AK022231.1 | STAT1 |
| NM_173624.1 | FLJ40504 |
| NM_001353.4 | AKR1C1 |
| NM_020529.1 | NFKBIA |
| NM_014338.3 | PISD |
| NM_003243.1 | TGFBR3 |
| NM_004951.2 | EBI2 |
| NM_002350.1 | LYN |
| NM_079421.2 | CDKN2D |
| U90916.1 | SORL1 |
| NM_005213.2 | CSTA |
| NM_002155.3 | HSPA6 |
| NM_004126.2 | GNG11 |
| NM_023037.1 | 13CDNA73 |
| AI597616.1 | MRPL33 |
| NM_007360.1 | KLRK1 |
| AW291384.1 | STS-1 |

TABLE 13
An HCP set specific for MCL, according to one embodiment
of the invention

| MCL | |
|---|---|
| Signature | |
| Accession | Symbol |
| NM_004851.1 | NAPSA |
| NM_004619.2 | TRAF5 |
| NM_015187.1 | KIAA0746 |
| NM_002250.2 | KCNN4 |
| NM_012452.2 | TNFRSF13B |
| NM_006317.3 | BASP1 |
| NM_017530.1 | LOC55565 |
| NM_017784.3 | OSBPL10 |
| R99527.1 | MGC39372 |
| NM_182776.1 | MCM7 |
| NM_003177.3 | SYK |
| L29376.1 | 3.8-1 |
| NM_001770.3 | CD19 |
| NM_002661.1 | PLCG2 |
| NM_201593.1 | CACNB2 |
| NM_001951.2 | E2F5 |
| NM_006162.3 | NFATC1 |
| NM_020992.2 | PDLIM1 |
| NM_002120.2 | HLA-DOB |
| NM_021950.2 | MS4A1 |
| NM_003362.2 | UNG |
| NM_175870.3 | LOC90925 |
| NM_000405.3 | GM2A |
| NM_002095.3 | GTF2E2 |
| NM_002646.2 | PIK3C2B |
| NM_001242.3 | TNFRSF7 |
| NM_014686.2 | KIAA0355 |
| AA594161.1 | MYH11 |
| NM_033274.1 | ADAM19 |
| NM_000626.1 | CD79B |
| NM_022552.3 | DNMT3A |
| NM_001877.2 | CR2 |
| U37028.1 | ITGAD |
| NM_014246.1 | CELSR1 |
| AA766908.1 | MME |
| BQ632574.1 | RASA1 |
| NM_013230.1 | CD24 |
| NM_003451.1 | ZNF177 |
| NM_000237.1 | LPL |
| NM_001618.2 | PARP1 |
| NM_001771.1 | CD22 |
| NM_004556.1 | NFKBIE |
| NM_030764.2 | SPAP1 |
| NM_031305.1 | ARHGAP24 |
| NM_017935.2 | BANK1 |
| NM_025113.1 | C13orf18 |
| AK055652.1 | C3orf6 |
| BG993697.1 | SMYD3 |
| NM_016187.1 | BIN2 |
| NM_138340.3 | ABHD3 |
| NM_003332.2 | TYROBP |
| NM_014338.3 | PISD |
| NM_012448.3 | STAT5B |
| NM_198833.1 | SERPINB8 |
| NM_001760.2 | CCND3 |
| NM_004184.3 | WARS |
| NM_005601.3 | NKG7 |
| NM_006889.2 | CD86 |
| NM_003037.1 | SLAMF1 |
| NM_003810.2 | TNFSF10 |
| U90916.1 | SORL1 |
| NM_005213.2 | CSTA |
| NM_002051.2 | GATA3 |
| NM_000043.3 | FAS |
| NM_005013.1 | NUCB2 |
| NM_001779.1 | CD58 |
| NM_030666.2 | SERPINB1 |
| NM_006620.2 | HBS1L |
| NM_001621.2 | AHR |
| NM_004635.3 | MAPKAPK3 |
| NM_023037.1 | 13CDNA73 |
| NM_000295.3 | SERPINA1 |
| AI597616.1 | MRPL33 |
| NM_002966.1 | S100A10 |
| NM_000576.2 | IL1B |
| NM_006498.1 | LGALS2 |
| NM_006257.2 | PRKCQ |
| NM_003656.3 | CAMK1 |
| NM_004529.1 | MLLT3 |
| NM_006332.3 | IFI30 |

TABLE 14
An HCP set specific for MZL, according to one embodiment
of the invention

| MZL | |
|---|---|
| Signature | |
| Accession | Symbol |
| NM_004619.2 | TRAF5 |
| NM_006274.2 | CCL19 |
| NM_003959.1 | HIP1R |
| NM_014257.3 | CD209L |
| NM_015187.1 | KIAA0746 |
| NM_001894.4 | CSNK1E |
| NM_002250.2 | KCNN4 |
| NM_012452.2 | TNFRSF13B |
| NM_006317.3 | BASP1 |
| NM_006558.1 | KHDRBS3 |
| NM_017530.1 | LOC55565 |
| NM_017784.3 | OSBPL10 |
| NM_145804.1 | ABTB2 |
| NM_014762.1 | DHCR24 |
| NM_182776.1 | MCM7 |
| NM_004445.1 | EPHB6 |
| NM_001888.1 | CRYM |
| NM_003226.2 | TFF3 |
| NM_003177.3 | SYK |
| NM_001853.2 | COL9A3 |
| NM_000075.2 | CDK4 |
| NM_002002.3 | FCER2 |
| NM_001770.3 | CD19 |
| NM_000483.3 | APOC2 |
| NM_000109.2 | DMD |
| NM_006986.2 | MAGED1 |
| NM_003088.2 | FSCN1 |
| NM_001951.2 | E2F5 |
| NM_006841.3 | SLC38A3 |
| NM_006761.3 | YWHAE |
| NM_004073.2 | PLK3 |
| NM_006162.3 | NFATC1 |
| NM_002120.2 | HLA-DOB |
| NM_002738.5 | PRKCB1 |
| NM_021950.2 | MS4A1 |
| NM_000269.2 | NME1 |
| NM_175870.3 | LOC90925 |
| NM_000405.3 | GM2A |
| NM_002217.1 | ITIH3 |
| NM_000853.1 | GSTT1 |
| NM_021966.1 | TCL1A |
| NM_002646.2 | PIK3C2B |
| NM_144646.2 | IGJ |
| NM_001242.3 | TNFRSF7 |
| NM_030775.2 | WNT5B |
| NM_000626.1 | CD79B |
| NM_001877.2 | CR2 |
| NM_004422.2 | DVL2 |
| U37028.1 | ITGAD |
| NM_006892.3 | DNMT3B |
| AI809213.1 | RGS13 |
| AI634809.1 | ARID5B |
| AA766908.1 | MME |
| BQ632574.1 | RASA1 |
| NM_173624.1 | FLJ40504 |
| NM_013230.1 | CD24 |
| NM_001647.1 | APOD |
| AK022293.1 | CTSD |
| NM_080881.1 | DBN1 |
| NM_001782.1 | CD72 |
| NM_000237.1 | LPL |
| NM_004844.1 | SH3BP5 |
| NM_001197.3 | BIK |
| NM_006763.2 | BTG2 |
| NM_001353.4 | AKR1C1 |
| NM_000424.2 | KRT5 |
| NM_001771.1 | CD22 |
| NM_002832.2 | PTPN7 |
| NM_002891.3 | RASGRF1 |
| AW271958.1 | TYMS |
| NM_025216.2 | WNT10A |
| NM_030764.2 | SPAP1 |
| NM_031942.3 | CDCA7 |
| X58529.1 | IGHM |
| NM_001852.3 | COL9A2 |
| NM_018842.3 | BAIAP2L1 |
| NM_025113.1 | C13orf18 |
| NM_002122.2 | HLA-DQA1 |
| AK055652.1 | C3orf6 |
| NM_000271.1 | NPC1 |
| NM_138340.3 | ABHD3 |
| NM_003332.2 | TYROBP |
| NM_020199.1 | C5orf15 |
| NM_004117.2 | FKBP5 |
| NM_004289.5 | NFE2L3 |
| NM_177968.1 | PPM1B |
| NM_015641.2 | TES |
| NM_080593.1 | HIST1H2BK |
| NM_015866.2 | PRDM2 |
| T66903.1 | LOC54103 |
| NM_001154.2 | ANXA5 |
| NM_002467.2 | MYC |
| NM_005935.1 | MLLT2 |
| NM_001762.2 | CCT6A |
| NM_000313.1 | PROS1 |
| NM_000927.3 | ABCB1 |
| NM_002350.1 | LYN |
| NM_005271.1 | GLUD1 |
| NM_000917.1 | P4HA1 |
| NM_000655.2 | SELL |
| NM_000788.1 | DCK |
| NM_000038.2 | APC |
| M80899.1 | MGC5395 |
| NM_172373.2 | ELF1 |
| NM_001760.2 | CCND3 |
| NM_001663.2 | ARF6 |
| NM_004184.3 | WARS |
| NM_005461.3 | MAFB |
| NM_005601.3 | NKG7 |
| NM_006889.2 | CD86 |
| NM_003875.1 | GMPS |
| NM_032991.1 | CASP3 |
| NM_003810.2 | TNFSF10 |
| NM_004354.1 | CCNG2 |
| NM_003915.2 | RBM12 |
| U90916.1 | SORL1 |
| NM_000397.2 | CYBB |
| NM_005348.2 | HSPCA |
| NM_002526.1 | NT5E |
| NM_001068.2 | TOP2B |
| NM_005610.1 | RBBP4 |
| NM_005013.1 | NUCB2 |
| NM_005638.3 | SYBL1 |
| NM_005127.2 | CLECSF2 |
| NM_002838.2 | PTPRC |
| NM_006006.3 | ZBTB16 |
| NM_006218.1 | PIK3CA |
| NM_002306.1 | LGALS3 |
| NM_139276.2 | STAT3 |
| NM_000627.2 | LTBP1 |
| NM_030666.2 | SERPINB1 |
| NM_002967.2 | SAFB |
| NM_003217.1 | TEGT |
| NM_018442.1 | IQWD1 |
| NM_004125.2 | GNG10 |
| NM_181838.1 | UBE2D2 |
| W56129.1 | SFRS9 |
| NM_002211.2 | ITGB1 |
| NM_004126.2 | GNG11 |
| NM_000584.2 | IL8 |
| NM_006195.2 | PBX3 |
| NM_023037.1 | 13CDNA73 |
| NM_000295.3 | SERPINA1 |
| NM_004049.2 | BCL2A1 |
| NM_002966.1 | S100A10 |
| NM_000585.2 | IL15 |
| NM_003656.3 | CAMK1 |
| NM_000579.1 | CCR5 |
| NM_001967.2 | EIF4A2 |
| NM_002752.3 | MAPK9 |
| NM_002892.2 | ARID4A |
| NM_002943.2 | RORA |
| NM_006164.2 | NFE2L2 |
| NM_006644.2 | HSPH1 |
| NM_007360.1 | KLRK1 |
| NM_016628.2 | WAC |
| NM_021822.1 | APOBEC3G |
| NM_024708.2 | ASB7 |
| NM_000713.1 | BLVRB |

TABLE 15
An HCP set specific for SLL, according to one embodiment
of the invention

| SLL | |
|---|---|
| Signature | |
| Accession | Symbol |
| NM_004619.2 | TRAF5 |
| NM_006274.2 | CCL19 |
| NM_014257.3 | CD209L |
| NM_015187.1 | KIAA0746 |
| NM_001894.4 | CSNK1E |
| NM_002250.2 | KCNN4 |
| NM_017530.1 | LOC55565 |
| NM_017784.3 | OSBPL10 |
| NM_006734.2 | HIVEP2 |
| NM_003226.2 | TFF3 |
| NM_003177.3 | SYK |
| L29376.1 | 3.8-1 |
| NM_001853.2 | COL9A3 |
| NM_000075.2 | CDK4 |
| NM_002002.3 | FCER2 |
| NM_001770.3 | CD19 |
| NM_000109.2 | DMD |
| NM_201593.1 | CACNB2 |
| NM_001951.2 | E2F5 |
| NM_006841.3 | SLC38A3 |
| NM_004073.2 | PLK3 |
| NM_006162.3 | NFATC1 |
| NM_002120.2 | HLA-DOB |
| NM_002738.5 | PRKCB1 |
| NM_021950.2 | MS4A1 |
| NM_003362.2 | UNG |
| NM_000269.2 | NME1 |
| NM_021966.1 | TCL1A |
| NM_001242.3 | TNFRSF7 |
| H53164.1 | IRF8 |
| AA594161.1 | MYH11 |
| NM_000626.1 | CD79B |
| NM_001877.2 | CR2 |
| NM_006892.3 | DNMT3B |
| AI634809.1 | ARID5B |
| BQ632574.1 | RASA1 |
| AI672553.1 | AKAP12 |
| NM_002567.2 | PBP |
| NM_173624.1 | FLJ40504 |
| NM_013230.1 | CD24 |
| NM_003451.1 | ZNF177 |
| AK022293.1 | CTSD |
| NM_005449.3 | TOSO |
| NM_001782.1 | CD72 |
| NM_000237.1 | LPL |
| NM_004844.1 | SH3BP5 |
| NM_001197.3 | BIK |
| NM_001353.4 | AKR1C1 |
| NM_001552.1 | IGFBP4 |
| NM_001771.1 | CD22 |
| NM_002692.2 | POLE2 |
| NM_002832.2 | PTPN7 |
| NM_002891.3 | RASGRF1 |
| NM_004556.1 | NFKBIE |
| NM_025216.2 | WNT10A |
| NM_030764.2 | SPAP1 |
| NM_001852.3 | COL9A2 |
| NM_001238.1 | CCNE1 |
| NM_025113.1 | C13orf18 |
| NM_002122.2 | HLA-DQA1 |
| AK055652.1 | C3orf6 |
| AL080130.1 | FLJ14001 |
| NM_000271.1 | NPC1 |
| NM_138340.3 | ABHD3 |
| NM_003332.2 | TYROBP |
| NM_004117.2 | FKBP5 |
| NM_001196.2 | BID |
| NM_015641.2 | TES |
| NM_012448.3 | STAT5B |
| NM_181443.1 | BTBD3 |
| NM_006254.3 | PRKCD |
| H57732.1 | TGFBR2 |
| NM_001154.2 | ANXA5 |
| NM_002467.2 | MYC |
| NM_004951.2 | EBI2 |
| NM_005935.1 | MLLT2 |
| NM_005415.3 | SLC20A1 |
| NM_000927.3 | ABCB1 |
| NM_000917.1 | P4HA1 |
| NM_004184.3 | WARS |
| NM_005601.3 | NKG7 |
| NM_006889.2 | CD86 |
| NM_032991.1 | CASP3 |
| NM_003810.2 | TNFSF10 |
| NM_001166.3 | BIRC2 |
| NM_004354.1 | CCNG2 |
| NM_003915.2 | RBM12 |
| U90916.1 | SORL1 |
| NM_005211.2 | CSF1R |
| NM_000397.2 | CYBB |
| NM_005213.2 | CSTA |
| NM_002051.2 | GATA3 |
| NM_001068.2 | TOP2B |
| NM_005013.1 | NUCB2 |
| NM_005638.3 | SYBL1 |
| NM_002838.2 | PTPRC |
| NM_006006.3 | ZBTB16 |
| NM_001110.1 | ADAM10 |
| NM_002306.1 | LGALS3 |
| NM_000627.2 | LTBP1 |
| NM_030666.2 | SERPINB1 |
| NM_002967.2 | SAFB |
| NM_003217.1 | TEGT |
| NM_018442.1 | IQWD1 |
| W56129.1 | SFRS9 |
| NM_002211.2 | ITGB1 |
| NM_000584.2 | IL8 |
| NM_000295.3 | SERPINA1 |
| NM_004049.2 | BCL2A1 |
| NM_002966.1 | S100A10 |
| NM_003299.1 | TRA1 |
| NM_000576.2 | IL1B |
| NM_006498.1 | LGALS2 |
| NM_000585.2 | IL15 |
| NM_001831.2 | CLU |
| NM_003656.3 | CAMK1 |
| NM_003254.1 | TIMP1 |
| NM_002305.2 | LGALS1 |
| NM_000579.1 | CCR5 |
| NM_001967.2 | EIF4A2 |
| NM_002943.2 | RORA |
| NM_004529.1 | MLLT3 |
| NM_006164.2 | NFE2L2 |
| NM_006332.3 | IFI30 |
| NM_006644.2 | HSPH1 |
| BI769730.1 | HLA-DRB1 |
| NM_005347.2 | HSPA5 |
| NM_000189.4 | HK2 |

TABLE 16
An HCP set specific for TCL, according to one embodiment
of the invention

| TCL | |
|---|---|
| Signature | |
| Accession No. | Symbol |
| NM_006274.2 | CCL19 |
| NM_005856.1 | RAMP3 |
| NM_004577.3 | PSPH |
| NM_004737.2 | LARGE |
| AF196185.1 | PARD3 |
| NM_017955.2 | CDCA4 |
| NM_014762.1 | DHCR24 |
| NM_006475.1 | POSTN |
| NM_004091.2 | E2F2 |
| NM_016076.2 | PNAS-4 |
| NM_078467.1 | CDKN1A |
| NM_002189.2 | IL15RA |
| NM_006841.3 | SLC38A3 |
| NM_002358.2 | MAD2L1 |
| NM_001254.2 | CDC6 |
| NM_000600.1 | IL6 |
| NM_002105.1 | H2AFX |
| NM_000269.2 | NME1 |
| NM_001826.1 | CKS1B |
| NM_001827.1 | CKS2 |
| NM_182746.1 | MCM4 |
| NM_000853.1 | GSTT1 |
| NM_007361.2 | NID2 |
| NM_004460.2 | FAP |
| NM_016081.2 | KIAA0992 |
| NM_003290.1 | TPM4 |
| NM_030775.2 | WNT5B |
| NM_004322.2 | BAD |
| NM_005623.2 | CCL8 |
| NM_006892.3 | DNMT3B |
| NM_005950.1 | MT1G |
| NM_080881.1 | DBN1 |
| NM_005951.1 | MT1H |
| NM_003254.1 | TIMP1 |
| NM_001353.4 | AKR1C1 |
| NM_002692.2 | POLE2 |
| NM_006014.2 | DXS9879E |
| NM_016336.2 | UBE2J1 |
| NM_002122.2 | HLA-DQA1 |
| NM_000075.2 | CDK4 |
| NM_004851.1 | NAPSA |
| AB014540.1 | SWAP70 |
| NM_015641.2 | TES |
| NM_015866.2 | PRDM2 |
| NM_014456.3 | PDCD4 |
| T66903.1 | LOC54103 |
| NM_139207.1 | NAP1L1 |
| H57732.1 | TGFBR2 |
| NM_003648.2 | DGKD |
| NM_005582.1 | LY64 |
| NM_003243.1 | TGFBR3 |
| NM_004951.2 | EBI2 |
| NM_005935.1 | MLLT2 |
| NM_003177.3 | SYK |
| NM_002002.3 | FCER2 |
| NM_002350.1 | LYN |
| NM_000655.2 | SELL |
| NM_005531.1 | IFI16 |
| NM_005077.3 | TLE1 |
| NM_003810.2 | TNFSF10 |
| NM_004354.1 | CCNG2 |
| NM_006162.3 | NFATC1 |
| U90916.1 | SORL1 |
| NM_000397.2 | CYBB |
| NM_021950.2 | MS4A1 |
| NM_175870.3 | LOC90925 |
| NM_005013.1 | NUCB2 |
| NM_021966.1 | TCL1A |
| NM_005127.2 | CLECSF2 |
| NM_002838.2 | PTPRC |
| NM_001781.1 | CD69 |
| NM_000627.2 | LTBP1 |
| NM_030666.2 | SERPINB1 |
| NM_001706.2 | BCL6 |
| NM_002967.2 | SAFB |
| NM_003217.1 | TEGT |
| NM_000061.1 | BTK |
| W56129.1 | SFRS9 |
| NM_004271.1 | LY86 |
| NM_002211.2 | ITGB1 |
| NM_000626.1 | CD79B |
| NM_023037.1 | 13CDNA73 |
| NM_004510.2 | SP110 |
| AL833316.1 | MIR |
| NM_001967.2 | EIF4A2 |
| NM_006164.2 | NFE2L2 |
| NM_007360.1 | KLRK1 |
| NM_020151.2 | STARD7 |
| NM_021813.1 | BACH2 |
| NM_030764.2 | SPAP1 |
| BE897089.1 | ZNF91 |
| NM_017935.2 | BANK1 |

TABLE 17
An HCP set specific for CLL, according to one embodiment of the
invention

| | GenBank ™ |
|---|---|
| Symbol | Accession No. |
| BAIAP2L1 | NM_018842.3 |
| C3orf6 | AK055652.1 |
| CD74 | NM_004355.1 |
| CDCA7 | NM_031942.3 |
| CDKN3 | NM_005192.2 |
| CLIC5 | NM_016184.2 |
| CNN3 | AL049313.1 |
| COL1A2 | NM_001839.2 |
| CX3CR1 | NM_000089.3 |
| ENPP2 | NM_001337.2 |
| GATA3 | NM_006209.2 |
| HCK | NM_002051.2 |
| IGFBP7 | NM_002110.2 |
| ITGAL | NM_001553.1 |
| LGALS2 | T28925.1 |
| LGALS3 | NM_006498.1 |
| LOC54103 | NM_002306.1 |
| MT1H | AI523104.1 |
| NK4 | T66903.1 |
| NKG7 | NM_005951.1 |
| PLA2G7 | NM_004221.3 |
| PLAU | NM_005601.3 |
| PON2 | NM_005084.2 |
| PPBP | NM_002658.1 |
| PRKCD | NM_000305.1 |
| SPAP1 | NM_002704.2 |
| SPIB | NM_006254.3 |
| STAT1 | NM_030764.2 |
| TCF4 | NM_003121.1 |
| TLE1 | AK022231.1 |
| TNFSF10 | AI307786.1 |
| TRIP6 | NM_005077.3 |
| TYROBP | NM_003810.2 |
| CLECSF6 | NM_003302.1 |
| LILRB1 | NM_003332.2 |
| ANXA1 | NM_000700.1 |
| BCL2A1 | NM_000484.2 |
| CCNA2 | NM_004049.2 |
| CD24 | NM_001237.2 |
| CDC2 | NM_013230.1 |
| CSF3R | NM_001786.2 |
| ENTPD1 | NM_156038.2 |
| FN1 | NM_001776.3 |
| APP | NM_212474.1 |
| STMN1 | NM_005531.1 |
| IFI16 | NM_001558.2 |
| IL10RA | NM_007360.1 |
| KLRK1 | NM_000237.1 |
| LPL | NM_002341.1 |
| LTB | NM_002358.2 |
| MAD2L1 | NM_021038.3 |
| MBNL1 | NM_002619.1 |
| PF4 | NM_002692.2 |
| POLE2 | NM_006115.3 |
| PRAME | NM_004577.3 |
| PSPH | NM_005608.1 |
| PTPRCAP | NM_015149.2 |
| RGL1 | NM_003821.4 |
| RIPK2 | NM_005621.1 |
| S100A12 | NM_006748.1 |
| SLA | U90916.1 |
| SORL1 | NM_005563.3 |
| SWAP70 | AB014540.1 |
| TFRC | NM_003234.1 |
| TRAF1 | NM_005658.2 |
| ZAP70 | NM_001079.3 |

TABLE 18
An HCP set specific for AML, according to one embodiment of the
invention

| | GenBank ™ |
|---|---|
| Symbol | Accession No. |
| BIRC5 | NM_001168.1 |
| BZRP | NM_007311.2 |
| C13orf18 | NM_025113.1 |
| C3orf6 | AK055652.1 |
| CDC20 | NM_001255.1 |
| CDC6 | NM_001254.2 |
| CDCA7 | NM_031942.3 |
| CDKN3 | NM_005192.2 |
| CHEK1 | NM_001274.2 |
| CLIC5 | AL049313.1 |
| CSF1R | NM_005211.2 |
| DMD | NM_000109.2 |
| FGR | NM_005248.1 |
| HCK | NM_002110.2 |
| IL16 | NM_004513.3 |
| KNTC2 | NM_006101.1 |
| LGALS1 | NM_002305.2 |
| LGALS3 | NM_002306.1 |
| LILRB1 | AI523104.1 |
| NKG7 | NM_005601.3 |
| PCDH9 | BC042366.1 |
| PLA2G7 | NM_005084.2 |
| PLCG2 | NM_002661.1 |
| PON2 | NM_000305.1 |
| PPBP | NM_002704.2 |
| PRKCD | NM_006254.3 |
| PTPN6 | NM_002831.3 |
| PTPRC | NM_002838.2 |
| RAB13 | NM_002870.2 |
| RAG2 | NM_000536.1 |
| S100A6 | NM_014624.2 |
| SERPINA1 | NM_000295.3 |
| SFRS3 | BE927772.1 |
| SPAP1 | NM_030764.2 |
| SPIB | NM_003121.1 |
| TCF4 | AI307786.1 |
| TCF7 | NM_201632.1 |
| TIMP1 | NM_003254.1 |
| TLE1 | NM_005077.3 |
| AKAP12 | AI672553.1 |
| ANXA1 | NM_000700.1 |
| APOBEC3G | NM_021822.1 |
| ASPM | NM_018136.2 |
| AUTS2 | NM_015570.1 |
| BASP1 | NM_006317.3 |
| BCL2A1 | NM_004049.2 |
| BIRC3 | NM_001165.3 |
| BTK | NM_000061.1 |
| CASP4 | AL050391.1 |
| CCL5 | NM_002985.2 |
| CCNA2 | NM_001237.2 |
| CD2 | NM_001767.2 |
| CD24 | NM_013230.1 |
| CD36 | NM_001001547.1 |
| CD3D | NM_000732.2 |
| CD44 | NM_000610.3 |
| CD79B | NM_000626.1 |
| CD86 | NM_006889.2 |
| CDC2 | NM_001786.2 |
| CDCA1 | NM_031423.2 |
| CR2 | NM_001877.2 |
| CSF3R | NM_156038.2 |
| CSTA | NM_005213.2 |
| CTGF | NM_001901.1 |
| CX3CR1 | NM_001337.2 |
| CXCR4 | NM_003467.1 |
| DNTT | NM_004088.2 |
| EBI2 | NM_004951.2 |
| ENPP2 | NM_006209.2 |
| ENTPD1 | NM_001776.3 |
| FCER2 | NM_002002.3 |
| FLJ40504 | NM_173624.1 |
| GUCY1A3 | NM_000856.1 |
| HCLS1 | NM_005335.3 |
| HIST1H2AC | BC050602.1 |
| HIVEP2 | NM_006734.2 |
| HLA- | NM_006120.2 |
| DMA | |
| HLA- | NM_033554.2 |
| DPA1 | |
| HLA- | NM_002121.4 |
| DPB1 | |
| HLA- | NM_019111.2 |
| DRA | |
| IFI30 | NM_006332.3 |
| IFI44L | NM_006820.1 |
| IGJ | NM_144646.2 |
| IL10RA | NM_001558.2 |
| KIF2C | NM_006845.2 |
| KLRK1 | NM_007360.1 |
| KRT19 | NM_002276.3 |
| LCK | NM_005356.2 |
| LTB | NM_002341.1 |
| LY86 | NM_004271.1 |
| MAD2L1 | NM_002358.2 |
| MAL | NM_022438.1 |
| MBNL1 | NM_021038.3 |
| MGMT | NM_002412.2 |
| MS4A1 | NM_021950.2 |
| MT1G | NM_005950.1 |
| MYL4 | W04885.1 |
| NAPSA | NM_004851.1 |
| NCF4 | NM_013416.2 |
| P2RY5 | NM_005767.3 |
| PF4 | NM_002619.1 |
| PLAU | NM_002658.1 |
| POLE2 | NM_002692.2 |
| PRAME | NM_006115.3 |
| PSPH | NM_004577.3 |
| PTPRCAP | NM_005608.1 |
| RGL1 | NM_015149.2 |
| RIPK2 | NM_003821.4 |
| RNASE1 | NM_198232.1 |
| S100A12 | NM_005621.1 |
| S100A4 | NM_002961.2 |
| SAMHD1 | NM_015474.2 |
| SERPINB1 | NM_030666.2 |
| SERPING 1 | NM_000062.1 |
| SESN3 | BU929213.1 |
| SLA | NM_006748.1 |
| SLAMF1 | NM_003037.1 |
| SLC1A4 | NM_003038.2 |
| SORL1 | U90916.1 |
| STMN1 | NM_005563.3 |
| SWAP70 | AB014540.1 |
| TCL1A | NM_021966.1 |
| TFRC | NM_003234.1 |
| TOP2A | NM_001067.2 |
| TRIP6 | NM_003302.1 |
| TXNIP | NM_006472.1 |
| TYROBP | NM_003332.2 |
| VEGF | BC058855.1 |
| ZAP70 | NM_001079.3 |

TABLE 19
An HCP set specific for T-ALL, according to one embodiment of the
invention

| | GenBank ™ |
|---|---|
| Symbol | Accession No. |
| BIRC5 | NM_002102.2 |
| C13orf18 | NM_001168.1 |
| C3orf6 | NM_001255.1 |
| CD74 | NM_006101.1 |
| CDC20 | NM_001254.2 |
| CDC6 | NM_001274.2 |
| CHEK1 | NM_000536.1 |
| CLIC5 | NM_004088.2 |
| CSF1R | NM_201632.1 |
| DNTT | NM_005601.3 |
| FGR | NM_002838.2 |
| GYPE | AL049313.1 |
| IL16 | AI307786.1 |
| KNTC2 | NM_000305.1 |
| LILRB1 | AK055652.1 |
| NKG7 | NM_002870.2 |
| PCDH9 | NM_025113.1 |
| PLCG2 | NM_002661.1 |
| PON2 | NM_003121.1 |
| PTPRC | NM_004513.3 |
| RAB13 | AI523104.1 |
| RAG2 | NM_004355.1 |
| SERPINA1 | BC042366.1 |
| SPIB | NM_005248.1 |
| TCF4 | NM_001242.3 |
| TCF7 | NM_005211.2 |
| TNFRSF7 | NM_000295.3 |
| AKAP12 | W04885.1 |
| ANXA1 | NM_013230.1 |
| APP | NM_173624.1 |
| ASPM | BC058855.1 |
| AUTS2 | NM_002358.2 |
| BASP1 | NM_018136.2 |
| BCL2A1 | NM_006845.2 |
| BIRC3 | NM_031966.2 |
| BTK | NM_001237.2 |
| CASP4 | NM_001067.2 |
| CCL5 | NM_003234.1 |
| CCNA2 | NM_001786.2 |
| CCNB1 | NM_002692.2 |
| CD2 | NM_004577.3 |
| CD24 | NM_022438.1 |
| CD36 | NM_006748.1 |
| CD37 | NM_004615.2 |
| CD3D | NM_000700.1 |
| CD44 | NM_000732.2 |
| CD86 | NM_003467.1 |
| CDC2 | NM_001767.2 |
| CNN3 | NM_005356.2 |
| CSF3R | NM_006734.2 |
| CSTA | NM_001079.3 |
| CTGF | NM_005608.1 |
| CXCR4 | NM_005531.1 |
| EBI2 | NM_012151.2 |
| ENG | AI672553.1 |
| ENPP2 | BU929213.1 |
| ENTPD1 | NM_005965.3 |
| F8A1 | NM_000237.1 |
| FCER2 | NM_001839.2 |
| FLJ40504 | NM_006209.2 |
| FN1 | NM_000062.1 |
| HCK | NM_212474.1 |
| HCLS1 | NM_001901.1 |
| HIST1H2AC | NM_003302.1 |
| HIVEP2 | NM_003821.4 |
| HLA-DMA | BC050602.1 |
| HLA-DPA1 | NM_015149.2 |
| HLA-DPB1 | NM_021966.1 |
| HLA-DRA | NM_002002.3 |
| IFI16 | NM_006317.3 |
| IFI30 | NM_006120.2 |
| IFI44L | NM_001774.1 |
| IGJ | NM_144646.2 |
| IL10RA | NM_021950.2 |
| KIF2C | NM_002341.1 |
| KLRK1 | NM_013416.2 |
| LCK | NM_005213.2 |
| LPL | NM_015570.1 |
| LTB | U90916.1 |
| LY86 | NM_004049.2 |
| MAD2L1 | NM_015474.2 |
| MAL | NM_004951.2 |
| MBNL1 | NM_003037.1 |
| MS4A1 | NM_156038.2 |
| MYL4 | NM_001776.3 |
| MYLK | NM_004851.1 |
| NAPSA | NM_000484.2 |
| NCF4 | NM_006820.1 |
| POLE2 | NM_000061.1 |
| PSPH | NM_002985.2 |
| PTPRCAP | NM_001558.2 |
| RGL1 | NM_000610.3 |
| RIPK2 | NM_004271.1 |
| S100A12 | AB014540.1 |
| S100A4 | NM_001165.3 |
| SAMHD1 | NM_006889.2 |
| SERPING1 | NM_002110.2 |
| SESN3 | NM_005335.3 |
| SLA | NM_007360.1 |
| SLAMF1 | NM_000118.1 |
| SORL1 | AL050391.1 |
| SWAP70 | NM_001001547.1 |
| TCL1A | NM_005621.1 |
| TFRC | NM_021038.3 |
| TM4SF2 | NM_006332.3 |
| TOP2A | NM_002961.2 |
| TRIP6 | NM_006472.1 |
| TXNTP | NM_003332.2 |
| TYROBP | NM_033554.2 |
| VEGF | NM_002121.4 |
| ZAP70 | NM_019111.2 |

### 1.2 Polynucleotide Probes

The system of the present invention provides for combinations of polynucleotide probes (hematological cancer profiling (HCP) combinations) that are capable of detecting the genes of one or more HCP set. Each polynucleotide probe of the HCP combination comprises a nucleotide sequence derived from the nucleotide sequence of a gene within an HCP set (the target gene). The nucleotide sequence of the polynucleotide probe is designed such that it corresponds to, or is complementary to, a region that is unique to the target gene, or mRNA transcribed from the gene.

The polynucleotide probe can specifically hybridize under either stringent or lowered stringency hybridization conditions to a region of the target gene, to a mRNA transcribed from the target gene, or to a nucleic acid sequence (such as a cDNA) derived therefrom. If splice variants of a gene are known, the probe may be designed such that it hybridises to only a single splice variant (for example, comprising a sequence complementary to a region of the mRNA unique to that splice variant), or it may be designed such that it hybridises to all splice variants (for example, comprising a sequence complementary to a region of the mRNA common to all splice variants). When splice-variant specific probes are used, several different probes may be designed, each one specific for a different splice-variant.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the polynucleotide probe is complementary to a region of a target mRNA derived from a target gene in the HCP set. Computer programs can also be employed to select probe sequences that will not cross hybridize or will not hybridize non-specifically.

One skilled in the art will understand that the nucleotide sequence of the polynucleotide probe need not be identical to its target sequence in order to specifically hybridise thereto. The polynucleotide probes of the present invention, therefore, comprise a nucleotide sequence that is at least about 75% identical to a region of the target gene or mRNA. In another embodiment, the nucleotide sequence of the polynucleotide probe is at least about 90% identical a region of the target gene or mRNA. In a further embodiment, the nucleotide sequence of the polynucleotide probe is at least about 95% identical to a region of the target gene or mRNA. Methods of determining sequence identity are known in the art and can be determined, for example, by using the BLASTN program of the University of Wisconsin Computer Group (GCG) software or provided on the NCBI website. The nucleotide sequence of the polynucleotide probes of the present invention may exhibit variability by differing (e.g. by nucleotide substitution, including transition or transversion) at one, two, three, four or more nucleotides from the sequence of the target gene.

Other criteria known in the art may be employed in the design of the polynucleotide probes of the present invention. For example, the probes can be designed to have <50% G content and/or between about 25% and about 70% G+C content. Strategies to optimize probe hybridization to the target nucleic acid sequence can also be included in the process of probe selection. Hybridization under particular pH, salt, and temperature conditions can be optimized by taking into account melting temperatures and by using empirical rules that correlate with desired hybridization behaviours. Computer models may be used for predicting the intensity and concentration-dependence of probe hybridization.

As is known in the art, in order to represent a unique sequence in the human genome, a probe should be at least 15 nucleotides in length. Accordingly, the polynucleotide probes of the present invention range in length from about 15 nucleotides to the full length of the target gene or target mRNA. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides in length. Larger polynucleotide probes, for example, of about 525, 550, 575, 600, 625, 650, 675, or 700 nucleotides in length are also contemplated by the present invention. In further embodiments, the polynucleotide probes are between about 15 nucleotides and about 100 nucleotides, about 20 nucleotides and about 100 nucleotides, about 25 nucleotides and about 100 nucleotides, and about 25 nucleotides and about 75 nucleotides in length.

In one embodiment of the invention, each of the polynucleotide probes in an HCP combination comprises a sequence corresponding to or complementary to, the sequence of an mRNA transcribed from one of the genes listed in Table 1. Representative examples of suitable probe sequences include probes comprising all or a portion of one of the sequences as set forth in any one of SEQ ID NOs: 1-4530 (Tables 20-23, below). In one embodiment, the probes comprise at least 15 consecutive nucleotides of one of the sequences as set forth in SEQ ID NOs:1-4530 (Tables 20-23).

TABLE 20
30mer polynucleotide probes, according to one embodiment

| | | | |
|---|---|---|---|
| | GenBank Accession | | |
| SEQ ID NO: | No. for target gene | Symbol | 30mer |

| | | | |
|---|---|---|---|
| 1 | AA594161 | MYH11 | tcgagacgcagacttcaatggaaccaaggc |
| 2 | AA766908 | MME | caaagttttatcaagtttgggtctgtgctg |
| 3 | AB014540 | SWAP70 | acctgttaaagggccaacagaactcttggt |
| 4 | AF196185 | PARD3 | tggtgttaccagtgtacgcgcagtgacctt |
| 5 | AI597616 | MRPL33 | ttcagaaaccctgtagcgtgtaataaaaga |
| 6 | AI634809 | ARID5B | ctacttgcttccttttagcttctgccttgc |
| 7 | AI672553 | AKAP12 | catggcatgaaataagtcaggttctttaca |
| 8 | AI809213 | RGS13 | gaaacccacaaaatcagaaacacagtacaa |
| 9 | AK022231 | STAT1 | agatggcgagaacctaagtttcagttgatt |
| 10 | AK022293 | CTSD | gtttgtatccaactccgggcaatgtggggc |
| 11 | AK055652 | C3orf6 | atgaccctaataggtctgtttagtaacagt |
| 12 | AL080130 | FLJ14001 | ccacctgaattgagcatttaggaatttatg |
| 13 | AL833316 | MIR | tgctatacactgggatgtctaattgcagca |
| 14 | AW291384 | STS-1 | gggctacaatccattgttcttagtccaata |
| 15 | BE897089 | ZNF91 | catttatatcatcctcaaccctaaatggac |
| 16 | BG993697 | SMYD3 | aagaaatgcctataaagacagacttgagtg |
| 17 | BI769730 | HLA-DRB1 | gtacgaggagcatacgccacacttatgatt |
| 18 | BQ632574 | RASA1 | aacttctgactacttgttgtatctgctgga |
| 19 | BX504817 | SFRS7 | tgttgcacttggttagctttaattgttctg |
| 20 | H53164 | IRF8 | gaacctcccagtgaaagggcagccttcatt |
| 21 | H57732 | TGFBR2 | tagacagagtacaaggtgtaattcagacag |
| 22 | L29376 | 3.8-1 | atatgtataaaaacagtagtttgggggcct |
| 23 | M80899 | ARNAK | gtgtcggtgccaaaattggagggagattta |
| 24 | NM_000038 | APC | agatcagagggtgactgatgatacatgcat |
| 25 | NM_000043 | FAS | tactaactgctctcagagaaagtagctttg |
| 26 | NM_000061 | BTK | gtgggggtggaccgaatttggcaagaatga |
| 27 | NM_000075 | CDK4 | acactcgtgttcatgttggtgcgatcgggg |
| 28 | NM_000109 | DMD | aacaaggggttactttacatcctactaaga |
| 29 | NM_000189 | HK2 | cggggtgtgatgaatagcgaatcatctcaa |
| 30 | NM_000237 | LPL | caagaaagaaacgtacttaactgtgtgaag |
| 31 | NM_000269 | NME1 | cccgtcttaccgctagttccctgcatagca |
| 32 | NM_000271 | NPC1 | aaagaaagcatctcgtaggtgtgtctactg |
| 33 | NM_000295 | SERPINA1 | cccaaactaatctaggaatcactgtctaac |
| 34 | NM_000313 | PROS1 | cattcatagccagatgtgtatcagatgttt |
| 35 | NM_000397 | CYBB | caactaagttatgaaagtagagagatctgc |
| 36 | NM_000405 | GM2A | actatagaatcccatacacaacggaatcac |
| 37 | NM_000424 | KRT5 | tatccttttctggagagtagtctagaccaa |
| 38 | NM_000483 | APOC2 | cgaagcgtacggagcagtaagacacataag |
| 39 | NM_000576 | IL1B | attcgctcccacattctgatgagcaaccgc |
| 40 | NM_000579 | CCR5 | gagagtgcaacagtagcataggaccctacc |
| 41 | NM_000584 | IL8 | cagccaaaactccacagtcaatattagtaa |
| 42 | NM_000585 | IL15 | ggctctcattgacttccttactaagcatag |
| 43 | NM_000600 | IL6 | cagtacacccgcccgttcttactgctaaaa |
| 44 | NM_000626 | CD79B | cgtggaataaacggtgtgtcctgagaaacc |
| 45 | NM_000627 | LTBP1 | gatgatccccaatatatctaccattgtatg |
| 46 | NM_000633 | BCL2 | caccattagctataatggcactttgtttgt |
| 47 | NM_000655 | SELL | gtttccttttatgagacccattcctatttc |
| 48 | NM_000713 | BLVRB | agagagagtagcgtgcggactagcaacgca |
| 49 | NM_000785 | CYP27B1 | tctgccctagcctggtttacggtttcttat |
| 50 | NM_000788 | DCK | gtggaattaaaggaatccctagaataagga |
| 51 | NM_000853 | GSTT1 | gtggtgcactcctataaaaattgaagctgc |
| 52 | NM_000877 | IL1R1 | agcatgcatcacggatcaatagactgtact |
| 53 | NM_000917 | P4HA1 | tacgttgtcagaattggaatgacaaacagg |
| 54 | NM_000927 | ABCB1 | actttcatgtgactggagtcatcttgtcca |
| 55 | NM_001067 | TOP2A | cagcaatgagctattagattcattttggga |
| 56 | NM_001068 | TOP2B | atctttggtgaccctctcggttctgcttct |
| 57 | NM_001110 | ADAM10 | tttcttacgtacactgtacttctgtgtgca |
| 58 | NM_001154 | ANXA5 | cctggatgttaaacagatcaatctcactcc |
| 59 | NM_001166 | BIRC2 | tctcttatttctccccctagtttgtgagaa |
| 60 | NM_001196 | BID | ctgcctgctgtaagaccatcctctatggtt |
| 61 | NM_001230 | CASP10 | agtcctgtggttaacgccttcatttataga |
| 62 | NM_001238 | CCNE1 | acattatcagttgacagtgtacaatgcctt |
| 63 | NM_001242 | TNFRSF7 | gagtgttgtggccgtgaggacttttctgtc |
| 64 | NM_001254 | CDC6 | cataaggagactataactctacagattgtg |
| 65 | NM_001350 | DAXX | agggatggactaagctaatccccttttggt |
| 66 | NM_001425 | EMP3 | atagatcttgttcccccccgcacctttccc |
| 67 | NM_001552 | IGFBP4 | gaaggagacttaggaacctaccagttggcc |
| 68 | NM_001558 | IL10RA | atgtgccttgaacacaaagctctgtcaata |
| 69 | NM_001618 | PARP1 | gaaggaacgctaacaatttctcatacttag |
| 70 | NM_001621 | AHR | gatttagaccagaggtctagattaatactc |
| 71 | NM_001647 | APOD | taacagggtagggcatggttacatgtttag |
| 72 | NM_001663 | ARF6 | attattgaatgttgcaggtgagatgtggtt |
| 73 | NM_001706 | BCL6 | gcgtcatgcttgtgttataactactccgga |
| 74 | NM_001760 | CCND3 | ggaccatcatcctactgtaataaagatgat |
| 75 | NM_001762 | CCT6A | cacccaaattacgtttccacgagattattt |
| 76 | NM_001770 | CD19 | atctgaagacctcgagcagatgatgccaac |
| 77 | NM_001771 | CD22 | agacgtacgtatcagcggccccttgactct |
| 78 | NM_001779 | CD58 | gaataggagggtaattaagactcctggtct |
| 79 | NM_001781 | CD69 | tgtagacaggtccttttcgatggtacatat |
| 80 | NM_001782 | CD72 | ccccccaagaacttcccatggtagaatggg |
| 81 | NM_001826 | CKS1B | gatgaaacccctgcacactattgtcagtaa |
| 82 | NM_001827 | CKS2 | cataaaataatcgttccccggattctctca |
| 83 | NM_001831 | CLU | ggtcagaggtatttgcttaagtgatcataa |
| 84 | NM_001852 | COL9A2 | cttctctttctcgcataactccccacccct |
| 85 | NM_001853 | COL9A3 | cagattaatgactggctacagagtaacaaa |
| 86 | NM_001877 | CR2 | cactcagtgattgcaatttgcacaagtttt |
| 87 | NM_001888 | CRYM | aaagcaaagctaggtagccatttcttctgt |
| 88 | NM_001894 | CSNK1E | ctgggttttggattaacccatccttcctag |
| 89 | NM_001951 | E2F5 | aaaatgtgccaatgcctgtacattaacaga |
| 90 | NM_001967 | EIF4A2 | gcctgtaaccttacagtgtattcctaataa |
| 91 | NM_002002 | FCER2 | tgagtatccccagctcaggtggtgagtcct |
| 92 | NM_002051 | GATA3 | gatcactgcctttaatacagtctgttggaa |
| 93 | NM_002095 | GTF2E2 | gtttgcccatagtcttgaatgcaatatttg |
| 94 | NM_002101 | GYPC | actgagcatacccagtgggccaacaagcac |
| 95 | NM_002105 | H2AFX | gttccacccaagccaatttcagacccttcg |
| 96 | NM_002120 | HLA-DOB | agacaaatgctcaggtagtcactgtttcct |
| 97 | NM_002122 | HLA-DQA1 | agccacccggctacctaattcctcagtaac |
| 98 | NM_002155 | HSPA6 | gtcagggctatgctatgggccttctagact |
| 99 | NM_002189 | IL15RA | cagcgtttgggcttctaaccaccctgtctc |
| 100 | NM_002211 | ITGB1 | cactagtcacattcttgttttaagtgcctt |
| 101 | NM_002217 | ITIH3 | ggatggatggcccggattttatggcatctg |
| 102 | NM_002250 | KCNN4 | ttacaagtgcaggcgactggaggcaggact |
| 103 | NM_002305 | LGALS1 | ggagacttcaagattaagtgcgtggccttt |
| 104 | NM_002350 | LYN | tcaagcggttctgttcactaaatttttctc |
| 105 | NM_002358 | MAD2L1 | tgtcgacccacaatcactcacggcgcgtaa |
| 106 | NM_002426 | MMP12 | gtccattcttgcttgactctactattaagt |
| 107 | NM_002467 | MYC | ctcaaattggactttgggcataaaagaact |
| 108 | NM_002526 | NT5E | ggccagtaaaatagggtaaatcctattaga |
| 109 | NM_002567 | PBP | tgacgagaagagctagtaatcacctgcaca |
| 110 | NM_002646 | PIK3C2B | tgctcctgatttaaagagatctgtgtactg |
| 111 | NM_002661 | PLCG2 | aactggcatgagttggggtaatttcctatt |
| 112 | NM_002692 | POLE2 | tcactacgacaaataccgaatgcctctgca |
| 113 | NM_002738 | PRKCB1 | agttactgtgaactattgtctcttggagga |
| 114 | NM_002752 | MAPK9 | ctgcttctgaagttatctcttaatcctctt |
| 115 | NM_002832 | PTPN7 | acgactgtctatgccaatgaggctcggtgg |
| 116 | NM_002838 | PTPRC | gcatgctcgattattccctgtacaatattt |
| 117 | NM_002891 | RASGRF1 | tgaatttctgcaccgtttcagacttcgccc |
| 118 | NM_002892 | ARID4A | tgcccaggattgaaggtgtaaatgggacaa |
| 119 | NM_002943 | RORA | gtcatggtattcttcattactgtgcagttg |
| 120 | NM_002966 | S100A10 | gtccacttaccacccactttgtgtttcctt |
| 121 | NM_002967 | SAFB | gttctgttaggagttaccttaaactgtgta |
| 122 | NM_003037 | SLAMF1 | cctaggttcagatcttaacttggccactta |
| 123 | NM_003088 | FSCN1 | ctaaacgcccatgatagtagcttcaaactg |
| 124 | NM_003177 | SYK | cccaaggatgccttagcatgtgactcctga |
| 125 | NM_003217 | TEGT | cctgttaactctccataaatttggtgattc |
| 126 | NM_003226 | TFF3 | ggtggccacacataagctcatcagcataga |
| 127 | NM_003243 | TGFBR3 | aaagggcactcatttggattgccttacttc |
| 128 | NM_003254 | TIMP1 | gatctcataacgctggtataaggtggtctg |
| 129 | NM_003290 | TPM4 | cttggttatcctcttccaatgtgaggacac |
| 130 | NM_003299 | TRA1 | ttgttttggatgccccctaatccccttctc |
| 131 | NM_003332 | TYROBP | cccaatactctcctaaaataaacatgaagc |
| 132 | NM_003362 | UNG | gagttggctctacgcgaggtttgttaataa |
| 133 | NM_003451 | ZNF177 | ttaaaccactcttgtgggaagttcctcttt |
| 134 | NM_003648 | DGKD | ggtggggcttaccccctcgtatttataatc |
| 135 | NM_003656 | CAMK1 | tggcccccactctcatagatgtcatccagg |
| 136 | NM_003810 | TNFSF10 | actaaaagatcgcagtttgcctggtgcagt |
| 137 | NM_003875 | GMPS | caacacctggcaatgagatccctgtagagg |
| 138 | NM_003959 | HIP1R | aggaatgtcgcccattactcctcaggcctg |
| 139 | NM_004049 | BCL2A1 | gttgcggagttcataatgaataacacagga |
| 140 | NM_004073 | PLK3 | ccagctagggggcgttatttatggaccact |
| 141 | NM_004091 | E2F2 | ttatgagcaccatgtaagcctccttgtatt |
| 142 | NM_004117 | FKBP5 | aggctgcatatggattgccaagtcagcata |
| 143 | NM_004126 | GNG11 | tcacccaagagccataatacacctattata |
| 144 | NM_004184 | WARS | caggcctcatttcatcacgcagcatgtgca |
| 145 | NM_004271 | LY86 | gagatcaccgacatcatgacacagacacac |
| 146 | NM_004289 | NFE2L3 | aagatcacacttgtgggcaatctgggggag |
| 147 | NM_004322 | BAD | gagtccacaaactcgtcactcatcctccgg |
| 148 | NM_004354 | CCNG2 | cagtaatagataagtgagacagattgcttg |
| 149 | NM_004356 | CD81 | acaacatcctgactccgtcatttaataaag |
| 150 | NM_004358 | CDC25B | gggagcgttttgttgagcatgacagcctgc |
| 151 | NM_004422 | DVL2 | atccattgggccccttctaaccccagaatg |
| 152 | NM_004445 | EPHB6 | tgagaatgacgatacccgtgactcagccct |
| 153 | NM_004510 | SP110 | gttcagatcagataaagcttcttcgggctg |
| 154 | NM_004529 | MLLT3 | agaaggatttcttgtattggtgaaagacgg |
| 155 | NM_004556 | NFKBIE | ggtcacccctatgctggcatgaagagcatg |
| 156 | NM_004577 | PSPH | tcttagaaatacggattcggcagccttcag |
| 157 | NM_004619 | TRAF5 | agccttacgggagcacatgcgtttggtttt |
| 158 | NM_004635 | MAPKAPK3 | atgtcagataggacaacactgctgggtttt |
| 159 | NM_004737 | LARGE | agttgcaatgtgaccagcgaaagctgcatt |
| 160 | NM_004844 | SH3BP5 | tttccctaactccagttattctgctgctgc |
| 161 | NM_004851 | NAPSA | cgcggaggtcctgctacccagtaaaaatcc |
| 162 | NM_004951 | EBI2 | tttggaataaagagcaggatgctgcaaagt |
| 163 | NM_005013 | NUCB2 | aatatcatcaggtcatacagcagatggaac |
| 164 | NM_005077 | TLE1 | ttccttgcagttcacctcactttccgtcct |
| 165 | NM_005127 | CLECSF2 | cctcaaaactttactagggccaataaactt |
| 166 | NM_005211 | CSF1R | agtcacgccgtgggatgtctctgtccacat |
| 167 | NM_005213 | CSTA | caggtatcattttggctgattgcttctttg |
| 168 | NM_005214 | CTLA4 | ggcccaattcttacaaacatgtggttaatg |
| 169 | NM_005271 | GLUD1 | acagtagtgtccccatgaagtgctagataa |
| 170 | NM_005292 | GPR18 | cactaactactctggcgtcaatggatattc |
| 171 | NM_005347 | HSPA5 | ccacctagtctgtaactctttctgaggagc |
| 172 | NM_005348 | HSPCA | taaaagcctacctaagcatatcgtaaagct |
| 173 | NM_005415 | SLC20A1 | ccatgttcatttgtctacctcttaactgaa |
| 174 | NM_005449 | TOSO | gaacgtctacttatctcaagttctatgcta |
| 175 | NM_005461 | MAFB | gttgaaatcaaacctgccgcaacagaaatt |
| 176 | NM_005531 | IFI16 | caccatatatactagctgttaatcctatgg |
| 177 | NM_005582 | LY64 | catcagcccccacaatttgtcagagctgaa |
| 178 | NM_005601 | NKG7 | agctaccaccctcgtatttatcatacacac |
| 179 | NM_005606 | LGMN | ttttattgaagagggccgcaagccccaaat |
| 180 | NM_005610 | RBBP4 | tctctagcccactttatatcctgccaccag |
| 181 | NM_005623 | CCL8 | ctttggcaatcagtgctcctgtaagtcaaa |
| 182 | NM_005638 | SYBL1 | tgacatagatcttaggcccacttgaactct |
| 183 | NM_005652 | TERF2 | tgttcgtgtcttgcactggaattcaacatt |
| 184 | NM_005658 | TRAF1 | tggtaaccctgggcctattgaaagcaggat |
| 185 | NM_005856 | RAMP3 | tcatgatgctgtgcccgctatgggctgtgt |
| 186 | NM_005935 | MLLT2 | tgattttgtacatagggtagggaagcagtg |
| 187 | NM_005950 | MT1G | cacataccagcaccactactcacactacag |
| 188 | NM_005951 | MT1H | gcgaggaacacgcacacgttctgtgcaaac |
| 189 | NM_006006 | ZBTB16 | agagaagacgtacctctacctgtgctatgt |
| 190 | NM_006014 | DXS9879E | atatacgcgaatgaggcgccctatacccgc |
| 191 | NM_006115 | PRAME | tgttgacttgaggagttaatgtgatctttg |
| 192 | NM_006162 | NFATC1 | gtgcagcctttctcaggggactgtcattga |
| 193 | NM_006164 | NFE2L2 | tttgcctgtcacgtttgagtgttcctgggc |
| 194 | NM_006195 | PBX3 | acttactacctctgaacaatactcacgctg |
| 195 | NM_006218 | PIK3CA | cagggtttgatagcacttaaactagttcat |
| 196 | NM_006254 | PRKCD | tggaccagtctgcattcgctggcttctcct |
| 197 | NM_006257 | PRXCQ | aagatcaatggcccatgcatgctgtttgca |
| 198 | NM_006274 | CCL19 | gccgcagcagttaacctatgaccgtgcaga |
| 199 | NM_006317 | BASP1 | ttctcatgacgagttaatgcatgtccgtgg |
| 200 | NM_006332 | IFI30 | aaattccaccccatgatcaagaatcctgct |
| 201 | NM_006475 | POSTN | acataaaatgcacgcaagccattatctctc |
| 202 | NM_006498 | LGALS2 | aactggcttacgctgttggcacatttcctt |
| 203 | NM_006558 | KHDRBS3 | gattgacttacatactacagcacatgtact |
| 204 | NM_006620 | HBS1L | tattgaggaattctcatagctctgccttat |
| 205 | NM_006644 | HSPH1 | gtctgtagtctttatgatcctaaaagggaa |
| 206 | NM_006734 | HIVEP2 | acagagggtcgcacttggactctgagggtt |
| 207 | NM_006761 | YWHAE | ggtttggggagggacttcgttgtaatggtt |
| 208 | NM_006763 | BTG2 | ggaagaatgtacagcttatggacaaatgta |
| 209 | NM_006841 | SLC38A3 | tgaggtgacacagcctgtaggaacatacac |
| 210 | NM_006889 | CD86 | gccttgattagactcctagcacctggctag |
| 211 | NM_006892 | DNMT3B | cggatgcctagagtttaccttatgtttaat |
| 212 | NM_006986 | MAGED1 | tagagagccacatcctgttgactgaaagtg |
| 213 | NM_007311 | BZRP | ttctgcatgcttagagcatgttcttggaac |
| 214 | NM_007360 | KLRK1 | atttgtacttgcttgagtcccatgactgtt |
| 215 | NM_007361 | NID2 | tcctgtagtgcctctatagaagtacccaca |
| 216 | NM_012203 | GRHPR | ggacacatttgcgccaaaagtatggtaatt |
| 217 | NM_012448 | STAT5B | tcctatgcagagttacagtcacaaagttgt |
| 218 | NM_012452 | TNFRSF13B | cagtgcagctgtaggtcgtcatcacctaac |
| 219 | NM_013230 | CD24 | aatgaacactcttgctttattccagaatgc |
| 220 | NM_014246 | CELSR1 | agacctcttgatgttttcccactggtccct |
| 221 | NM_014257 | CD209L | tgcatttagggggcgggcttggtatgttgt |
| 222 | NM_014338 | PISD | ttgattcagggtctgtcttggaggcagagg |
| 223 | NM_014456 | PDCD4 | gttaagtaaaaggtggctggaacatctatt |
| 224 | NM_014686 | KIAA0355 | agactgtgtgaagccgtttgtgtggtctcc |
| 225 | NM_014762 | DHCR24 | gttttggatggagttaaacttgatgccagt |
| 226 | NM_015187 | KIAA0746 | tgcaatattgtgggtccaaactactctttg |
| 227 | NM_015641 | TES | gtccatggatatatcaaatgtcttcacttg |
| 228 | NM_015866 | PRDM2 | ccttcaacgctcccagacggtcaggaaaac |
| 229 | NM_016076 | PNAS-4 | gatatagcattgggctatcatgttacaaca |
| 230 | NM_016081 | KIAA0992 | tgctgggtttgggattaactagcattattt |
| 231 | NM_016187 | BIN2 | tctgtctttgacttacgacccattttgcaa |
| 232 | NM_016336 | UBE2J1 | agttgccttcagaactagtttgagctgctc |
| 233 | NM_016628 | WAC | gatttcttgtagatgtatccttcacgttgt |
| 234 | NM_017530 | LOC55565 | gtgggctctacgtcgacagaatgagccttt |
| 235 | NM_017784 | OSBPL10 | ttttagcctttacacatgaggtcaaaggag |
| 236 | NM_017935 | BANK1 | ccagtcattattgttacatcatgtttgcag |
| 237 | NM_017955 | CDCA4 | atcctcatggaccctagtttaaagggtatg |
| 238 | NM_018442 | IQWD1 | attttccctaaccttcaaatgtgggagctt |
| 239 | NM_018842 | BAIAP2L1 | ttctctgtttcacgtagttgggttgacaag |
| 240 | NM_020151 | STARD7 | gtctgtacagttactcatgtcattgtaatg |
| 241 | NM_020199 | C5orf15 | tcttgggcggacagtgtactttcctaatag |
| 242 | NM_020529 | NFKBIA | tacacttgtatgtggccgtccacggcagca |
| 243 | NM_020992 | PDLIM1 | aacccaatcaaatataacgtgtctgacact |
| 244 | NM_021813 | BACH2 | gcattgctatgaactaatggaattggaact |
| 245 | NM_021822 | APOBEC3G | caatttgaatcggttttgtaggtagaggaa |
| 246 | NM_021950 | MS4A1 | ctctaaatagccaacacccatctgtttttt |
| 247 | NM_021966 | TCL1A | actctgctcgccttagcactaccactcctg |
| 248 | NM_022552 | DNMT3A | tctcactttagccaattcgagggctccttg |
| 249 | NM_023037 | 13CDNA73 | tgaaaagggaattgtaaatggactgcaatg |
| 250 | NM_024708 | ASB7 | ttttgtaccctggtggtcgagtcttccctt |
| 251 | NM_025113 | C13orf18 | gtgaactgtaactgacaagatgaattactc |
| 252 | NM_025216 | WNT10A | gcgagacacaacacttgtcctcttggaggt |
| 253 | NM_030666 | SERPINB1 | aagacaagcttgcctcgcctgacttttctg |
| 254 | NM_030764 | SPAP1 | ctgaacctgcaatatcaccaaagtatgcat |
| 255 | NM_030775 | WNT5B | tctgccccaggtgtacggtttctctctgac |
| 256 | NM_031305 | ARHGAP24 | tgggttcaaatggtatacaatttgccagct |
| 257 | NM_031942 | CDCA7 | gaccttgaatcgataagtgtaaatacagct |
| 258 | NM_032991 | CASP3 | gctcaaaaaatgagctcgcatttgtcaatg |
| 259 | NM_033208 | TIGD7 | cagagattccacttcacactgaaagagatg |
| 260 | NM_033274 | ADAM19 | ttatttgggagattgttcacggaaaacaga |
| 261 | NM_057735 | CCNE2 | cttgctgtacataggctagctatttctaaa |
| 262 | NM_078467 | CDKN1A | ttctccacctagactgtaaacctctcgagg |
| 263 | NM_079421 | CDKN2D | tccagtctcttgtacagcgttttaaaagaa |
| 264 | NM_080593 | HIST1H2BK | tgttccgatagtgtctcgcgggcgaaacca |
| 265 | NM_138340 | ABHD3 | ccagtattgtgtctaaattctgtggatgat |
| 266 | NM_138379 | TIMD4 | tgactattaacggcgggcgaaacgaaaata |
| 267 | NM_139207 | NAP1L1 | agttttaagggacgtcagtgtttatgccat |
| 268 | NM_139276 | STAT3 | cccgtgggttgcttacctacctataaggtg |
| 269 | NM_144646 | IGJ | taagtcattgctgactgcatagctcttttt |
| 270 | NM_145804 | ABTB2 | ctgacacctgtcatatttatggaccagaca |
| 271 | NM_152785 | GCET2 | ggactaagacactacccatataattcccag |
| 272 | NM_172373 | ELF1 | cttgggctaatctggctgagtagtcagtta |
| 273 | NM_173624 | FLJ40504 | caaatctcatgaccttgccaaaatcttggc |
| 274 | NM_175870 | LOC90925 | cccgcggagtaacctggcgacatatttaga |
| 275 | NM_177968 | PPM1B | ctgtactgatagtacttcccagtatgatat |
| 276 | NM_181443 | BTBD3 | cagctaattcaagacactgtagtcacttgt |
| 277 | NM_181838 | UBE2D2 | tttcctaattgtaaactaggccaacctgaa |
| 278 | NM_182746 | MCM4 | acctcattggtaaagggctagagcctttct |
| 279 | NM_182776 | MCM7 | ctgcacttactccttttgctaataaaagtg |
| 280 | NM_198833 | SERPINB8 | ctcctgtcactcagtacaattattttgaga |
| 281 | NM_201593 | CACNB2 | caaaggtaactaggaaagcattcatctgct |
| 282 | R99527 | MGC39372 | tcgtctctgtagtgatgacttcctgtcctc |
| 283 | T66903 | LOC54103 | atttgcagcagggtttgtctttgtgaattc |
| 284 | U37028 | ITGAD | agaagaagacgaggtctacaatgccattcc |
| 285 | W56129 | SFRS9 | actgtgactgagtcggcccattctgtttag |
| 286 | X58529 | IGHM | tgggagtggtcgattatgtactactttgac |
| 287 | AA188785 | LOC286025 | aatgcagtatagttaaaatggcttcccaca |
| 288 | AA347638 | BCR | gaagggaacgtgaccggattccctcactgt |
| 289 | AA601122 | SECTM1 | aataaggttccgtccgcgtgctgggtcaga |
| 290 | AA642467 | SLC2A14 | gttctggtcaatcatgttgaaagcctcctt |
| 291 | AA682722 | LARP | tcatagaaggctctggtggtacctctgggc |
| 292 | AA757235 | LOC399933 | ccaactctgtggcataacctcaaagccata |
| 293 | AA765313 | FZD3 | ttgggttacttcagatgcctaacaccttcc |
| 294 | AB002310 | HUWE1 | gctatccaggagtgctctgaaggctttggg |
| 295 | AB002333 | ZNF518 | gctttgtgctccagatattctagttggaac |
| 296 | AB0244O2 | ING1 | cccaccatctgtttgaagattatatgaagt |
| 297 | AB028952 | SYNPO | agccttgagaaaccggcagttcccaagcca |
| 298 | AB032991 | NDFIP2 | tgtgaaattaggttggggttgcaacatctt |
| 299 | AF008915 | EVI5 | agtttttacatcacatccattgatctgcct |
| 300 | AF108138 | C15orf20 | acagagaacggaacttaggtgtgtctctac |
| 301 | AF116668 | LMO2 | tgactttgacgcttgcgaactagagatgtg |
| 302 | AF143327 | FLJ32810 | gctacagtggatctattcaaagcttaactt |
| 303 | AF200348 | D2S448 | gctgtaacaggagtggaatgtacatattta |
| 304 | AF245480 | MONDOA | cttcaaaatgctgacgctttggagagtaag |
| 305 | AO075159 | SPTA1 | agctattttgtatgcaactagtcactgctg |
| 306 | BU948323 | TCF4 | agcacaaaaatttagtctgctggctgactg |
| 307 | AI523104 | LILRB1 | tttcctataagaaatttagggcagggcacg |
| 308 | AI675029 | C7 | ctctgtattgttctgaccctggtaaatata |
| 309 | AI694012 | PSMA5 | tagtaaaaagtaataattaggcccaggcac |
| 310 | AI732248 | COL4A1 | gctagtattgccacagacgcattaaaagaa |
| 311 | AJ007509 | HNRPUL1 | cttccccttgactcatatgttttaacatga |
| 312 | AK022224 | ACTN1 | gtttgctagctcacttgtccatgaaaatat |
| 313 | AK023928 | PSCD1 | tggatgcattttgtggtgggccccctagag |
| 314 | AK024580 | NRP1 | gtttcaaaatgaacagcgaagccttaactt |
| 315 | AK025005 | MARCH1 | ctagtataccctgtcaatacaactttcatc |
| 316 | AK091853 | COL3A1 | actttgtaagcttgtatgtggttgttgatc |
| 317 | AK126650 | CAPZB | acatttccatagagaccgtgtggtttttgt |
| 318 | AK127124 | ARHGEF4 | ttagggaggagtgcctactacccaaagaaa |
| 319 | AK127132 | DRG1 | agaaacaggatgccccagtaaacatgattt |
| 320 | AK129940 | GNAI1 | tggtaagtagtttccaagttacgtgttgtc |
| 321 | AL049313 | CLIC5 | tttcctacaatctgactcaactgccaggtg |
| 322 | AL049327 | CBLB | ttgcttagatgatgacgttgtggattcatt |
| 323 | AL049449 | GAB1 | gatcaggtttagacaatgagctttggttgt |
| 324 | AL050391 | CASP4 | gcaccttcattagtacagcttgcatattta |
| 325 | AL137527 | LRCH3 | ttgctttcaccctgcggtattgttttccct |
| 326 | AL353942 | SEPT11 | atatcactagtgccaagacataaagcgggg |
| 327 | AL833915 | TDRD9 | gtgtctgggtgttacagtctgtgcccactg |
| 328 | AL834278 | USP30 | atagctgtgcaaagccttttaacctgactt |
| 329 | AU252485 | MAP7 | tggttgaactgctaggttgaatgagagact |
| 330 | AV650179 | PRG1 | gaaaaagctcccacagtatgtggcgtctga |
| 331 | AV686223 | KIAA1407 | cactcagtttgtcttctagtactcagctca |
| 332 | AW014009 | MAP2K3 | cgagccatttgtcccaagtgccaaagaagc |
| 333 | AW190768 | SLC13A3 | ctggggattccacagagttgacctctctga |
| 334 | AW292143 | GFAP | atgtacccagtattctaggtagtgccctat |
| 335 | AW439398 | CCND1 | cagcaccaacatgtaaccggcatgtttcca |
| 336 | AW452039 | HRMT1L3 | ccaaatgtaactcccacattgagtttatct |
| 337 | AW615076 | LOC4923O4 | aaggttcctcaattttgcgaaaggggcaac |
| 338 | AW664012 | ALOX5 | taagacttagaataaggaggtggggaatgt |
| 339 | AW967479 | CD38 | agaaacaacgcaaccgacgacgcacaacgc |
| 340 | AW970654 | PHTF2 | aactgttgtttctttgccacatgtgttgta |
| 341 | BC028066 | NALP1 | agcgctctaaccctgcatattatttacttg |
| 342 | BC034289 | LOC87769 | tggaataaatgacgacgtaaacgtagctgc |
| 343 | BC042366 | PCDH9 | ggggtgtttgatgactttgacagaataaat |
| 344 | BC045532 | LSM8 | catttccctgcttaccatagggattttaaa |
| 345 | BC050383 | TMPO | ccagcacacaattttgatttcaattagcct |
| 346 | BC050602 | HIST1H2AC | catgtgtatgaattgttggtgctctaaaga |
| 347 | BC058855 | VEGF | gcagtttttcgagatattccgtagtacata |
| 348 | BE147267 | ECE1 | ccatgtctatctcaggggcccgttacctct |
| 349 | BE549606 | BZW1 | ttaacaagttaatcaggcgctacaaatacc |
| 350 | BE798965 | LAMC1 | aattccaactgtggaccttttatatgtgcc |
| 351 | BE927772 | SFRS3 | ggtacattgtagagccgaactttgagttac |
| 352 | BF055235 | RIPK1 | ttactacttggccaccacgcagccttggct |
| 353 | BF513638 | WNT3 | acctgaggcttatctttgcacatgttaaag |
| 354 | BF848289 | CSDA | tgtggaagggagtcgttacgctgcagatcg |
| 355 | BG682263 | HDAC3 | atgaacgggtagacaaggactgagattgcc |
| 356 | BG683220 | PMS2 | cactgctggatgttgaaggtaacttaataa |
| 357 | BG747999 | MT1L | gagaatacaacaacgatgcggcacgaggaa |
| 358 | B1493513 | PHF8 | cctccctttcctatagagataaaagtgatt |
| 359 | B1559738 | GPR56 | atcagctgttgtaatcacctagcaaactgg |
| 360 | BM709325 | BCL7A | ccatcagtagatatggtttgtacaatgtac |
| 361 | BM711190 | MED28 | cacccatgagaattagacattattctcctt |
| 362 | BM727177 | GOLT1B | gtcttacctctcaataaaagggtacttttc |
| 363 | BQ009245 | GPM6B | gtggaaatagttgtggatgtactaagagta |
| 364 | BQ021469 | AGPS | ttagtgacccactgaccgtatttgaacaaa |
| 365 | BQ215285 | LEF1 | ctttgctagctaacctgactccgggtgagg |
| 366 | BQ898221 | ITGA6 | tttcgtaacacagcattgtatatgtgaagc |
| 367 | BU929213 | SESN3 | tgactaaagctgacctaaatggatcagttt |
| 368 | BU954396 | RPL11 | gggatcatccttcctggcaaataaattccc |
| 369 | BX095432 | CNTN5 | ttgatcgttggtgggttgctagcctttcag |
| 370 | BX640908 | EVI1 | tcagtgcattactatgggaggagcaactaa |
| 371 | BX648339 | USP53 | ccaactacttttcatgaagagtgctttgaa |
| 372 | BX648365 | SEPT7 | accaacctgtttctacctatatgcagtctc |
| 373 | BX649193 | TKT | ctgagaggaggggtaaatatatgttttgag |
| 374 | C05886 | ETV6 | cctgcaaaccagaacgactctagaatttcc |
| 375 | D31762 | TRAM2 | gacagtaaatataataccgctgtttccttg |
| 376 | D86967 | EDEM1 | attaccttagccacagggtggctgagcagg |
| 377 | H41942 | NPEPPS | tggtgtggcttaacggtatttcagaaggtc |
| 378 | M60028 | HLA-DQB1 | acacttttgatattagcccccgcgggaccc |
| 379 | M77810 | GATA2 | cctctctgaaatagccgaactccaggctgg |
| 380 | N26272 | MSI2 | aggataccagaccactgtaatcaataagaa |
| 381 | N28431 | COL6A1 | gatgtgttcgacgttttatcaaaggccccc |
| 382 | N41620 | WWP1 | caggtgcttgaacttgtcagtttgttttaa |
| 383 | N53163 | COL27A1 | ctggaagttggacctgcgtgcttcctctga |
| 384 | N54946 | EWSR1 | gcagagatcggccctactagatgcagagac |
| 385 | N70000 | UBE3B | cttatggattgagatgtgatcaaaggcttt |
| 386 | NM_000016 | ACADM | aaagacttattgtagcccgtgaacacattg |
| 387 | NM_000022 | ADA | atatccacgtcacggatgcttccatgcccg |
| 388 | NM_000024 | ADRB2 | tcccgagcaaaggtctaaagtttacagtaa |
| 389 | NM_000033 | ABCD1 | ttccgtgggatgccataaaagccgcccagt |
| 390 | NM_000062 | SERPING1 | catggccctgccatgctctccaaaccactt |
| 391 | NM_000089 | COL1A2 | ctgtattgagttgtatcgtgtggtgtattt |
| 392 | NM_000099 | CST3 | ctcaaataggccagaacaactcactaatta |
| 393 | NM_000110 | DPYD | gaacaaagctcaaagtatgcaatgcttcat |
| 394 | NM_000118 | ENG | gaacttgcctaactaactggcaggggagac |
| 395 | NM_000120 | EPHX1 | ccaactccgtgtggtaagcaacatggcttt |
| 396 | NM_000147 | FUCA1 | gccaaaattcgctatgtttacagtgatact |
| 397 | NM_000169 | GLA | aggaagctagggttctatgaatggacttca |
| 398 | NM_000182 | HADHA | accacggtgtaccgggatattaaacctctt |
| 399 | NM_000194 | HPRT1 | ttaccagtgaatctttgtcagcagttccct |
| 400 | NM_000214 | JAG1 | gattcccttgttattcaacagcaagtgtga |
| 401 | NM_000234 | LIG1 | tggacggaccccagggttattattgccttt |
| 402 | NM_000239 | LYZ | atagatcttaggcaaaataccagctgatga |
| 403 | NM_000246 | MHC2TA | aatcgtgacaacacttgtgttatgaagcat |
| 404 | NM_000249 | MLH1 | gttggtagcacttaagacttatacttgcct |
| 405 | NM_000251 | MSH2 | agcatgtaatagagtgtgctaaacagaaag |
| 406 | NM_000277 | PAH | gcataatggtaaatcttttggggtcatctt |
| 407 | NM_000305 | PON2 | tttattccagtaaggaacggcccttttagt |
| 408 | NM_000314 | PTEN | atgatgcacttattcaatagctgtcagccg |
| 409 | NM_000341 | SLC3A1 | cttgaatgtaaccgctttaagaaaggttct |
| 410 | NM_000361 | THBD | tcattaaatggggctcacaaaccatgcaaa |
| 411 | NM_000382 | ALDH3A2 | caatcctttgatcataaattctccccaact |
| 412 | NM_000395 | CSF2RB | cataagtggttaagcaagtattctcgttac |
| 413 | NM_000417 | IL2RA | acactggtagaacgtaaccacggaaaagag |
| 414 | NM_000418 | IL4R | ggttgagtaatgctcgtctgtgtgttttag |
| 415 | NM_000435 | NOTCH3 | agccttatggcagaatagaggtatttttag |
| 416 | NM_000448 | RAG1 | ataagaggcaggatatataagcgccagtga |
| 417 | NM_000484 | APP | gaatgattggcttgagatagattggtggaa |
| 418 | NM_000485 | APRT | tggatcccagggaaatatcagccttgggca |
| 419 | NM_000536 | RAG2 | ttggattatgcccatgaacaagttagtttc |
| 420 | NM_000545 | TCF1 | cttcagtgtaccgcgtctaccctgggattc |
| 421 | NM_000546 | TP53 | tgacaacctcttggtcgaccttagtaccta |
| 422 | NM_000561 | GSTM1 | tgagacgcacacacattctaacctcccgcc |
| 423 | NM_000574 | DAF | ctgaatcgagatgtccatagtcaaatttgt |
| 424 | NM_000578 | SLC11A1 | tgcggagcctgtaataattgagggacaggc |
| 425 | NM_000594 | TNF | ccctactattcagtggcgagaaataaagtt |
| 426 | NM_000598 | IGFBP3 | acacacgctgcatagagctctccttgaaaa |
| 427 | NM_000601 | HGF | catgcaagtgtaataacatatctcctgaag |
| 428 | NM_000604 | FGFR1 | ttttaaaagggtcgttaccagagatttacc |
| 429 | NM_000610 | CD44 | cctgggctaagtcatttaaactgggtcttt |
| 430 | NM_000628 | IL10RB | ccctggacttagccaccagagagctacatt |
| 431 | NM_000629 | IFNAR1 | ctttcaggattagttcgttccaaggaggct |
| 432 | NM_000640 | IL13RA2 | actcaacagtttccagtcatggccaaatgt |
| 433 | NM_000660 | TGFB1 | cattaatgaactcattcagtcaccatagca |
| 434 | NM_000700 | ANXA1 | atcaggattatggtttcccgttctgaaatt |
| 435 | NM_000701 | ATP1A1 | gtaaaaaaggaacacccggaaagactgaaa |
| 436 | NM_000717 | CA4 | gggactttaggcatgattaaaatatggaca |
| 437 | NM_000729 | CCK | tgagaggcagctgcgtttctgcaacctatg |
| 438 | NM_000732 | CD3D | ctgagagggacattactgatgggagtgagg |
| 439 | NM_000773 | CYP2E1 | gttacaaactctgtgtcattccccgctcat |
| 440 | NM_000778 | CYP4A11 | aacctggttttttcccgtgtgaggaatgcc |
| 441 | NM_000791 | DHFR | acccatgaaggtaactaacggaaggaaaaa |
| 442 | NM_000801 | FKBP1A | agagcgagccgaacacaaaggagacgaaca |
| 443 | NM_000852 | GSTP1 | actcacacgtatggaatacccctgcacgcg |
| 444 | NM_000856 | GUCY1A3 | gagttttttgtacgattcctgcaacttttc |
| 445 | NM_000867 | HTR2B | tagttatgtatagcagaactggcagttgtc |
| 446 | NM_000878 | IL2RB | ttaatgatccgtgtgtaccatctttgtgat |
| 447 | NM_000885 | ITGA4 | cgagtggaccattatcactttaaagccctt |
| 448 | NM_000887 | ITGAX | cgatttcccaggctgaattgggagtgagat |
| 449 | NM_000905 | NPY | cgaaaacgcctgacacgaagagcgagacaa |
| 450 | NM_000906 | NPR1 | gaaaagagactaggtgaagagagggcaggg |
| 451 | NM_000953 | PTGDR | ggcatacaaggagtatgatttaacagtatg |
| 452 | NM_000960 | PTGIR | tgatgacgtaggacatgtgcttggtacaaa |
| 453 | NM_000964 | RARA | gctaatgaagattactgacctgcgaagcat |
| 454 | NM_000967 | RPL3 | ggtggtcttgatgccttaggcctctcttgt |
| 455 | NM_000968 | RPL4 | tcttgcgtggtgctcgaagggctctttgga |
| 456 | NM_000970 | RPL6 | aagagagtggtgccgtccgacaaaacacgg |
| 457 | NM_000972 | RPL7A | gccaaacagaattaacgcaactaataacct |
| 458 | NM_001001547 | CD36 | cctgcatataccaatagcattacctatgac |
| 459 | NM_001005 | RPS3 | aaggagagcaggtagagcgaagcagacgag |
| 460 | NM_001009 | RPS5 | catagagactaactagcggggagaggagca |
| 461 | NM_001015 | RPS11 | catatattcatgtgtcatcgttcaggaaca |
| 462 | NM_001019 | RPS15A | gcactatgttgaactattaaacttaccagc |
| 463 | NM_001069 | TUBB2 | gttcttggagtcgaacatctgctgggtgag |
| 464 | NM_001079 | ZAP70 | tcaggtggtcaggcgtagatcaccagaata |
| 465 | NM_001092 | ABR | attgatgtaaaccacgtgagaggaaaatgt |
| 466 | NM_001130 | AES | gctgtatcaagggtgggcttagctgtgcct |
| 467 | NM_001150 | ANPEP | tccctaaagaccctaaatctgaggaatcaa |
| 468 | NM_001165 | BIRC3 | cattgtactaataccgggaacatgaagcca |
| 469 | NM_001175 | ARHGDIB | cccaactgatatgttcatagaatgacagtt |
| 470 | NM_001178 | ARNTL | gcactccagctaacaaaagattgacaataa |
| 471 | NM_001207 | BTF3 | cattctttgcagctaattaagccgaagaag |
| 472 | NM_001211 | BUB1B | agatgcactaccattgctgttctacttttt |
| 473 | NM_001219 | CALU | aatgcgaacaggtacctatctgtttctaaa |
| 474 | NM_001226 | CASP6 | atcgttagggtgaagcattatggtctaatg |
| 475 | NM_001228 | CASP8 | gtgaactatgaagtaagcaacaaggatgac |
| 476 | NM_001229 | CASP9 | agcaaaccggggcctactagagtctgaaca |
| 477 | NM_001237 | CCNA2 | gttgtggccaagtacaaattatggtatcta |
| 478 | NM_001239 | CCNH | tctgcataatcaaaggggtagcacaaataa |
| 479 | NM_001255 | CDC20 | aagcgaagagcaacgcacgagagaaacgac |
| 480 | NM_001256 | CDC27 | ctccatacagtgtactcttattcttggcaa |
| 481 | NM_001265 | CDX2 | gtctgtgtaacatccaagccagagttttta |
| 482 | NM_001266 | CES1 | aagactggggtcttttgcgaaagggattgc |
| 483 | NM_001273 | CHD4 | ataccacctccaccgctgagcagtgacctt |
| 484 | NM_001274 | CHEK1 | gcaaactgcaaatagtagttcctgaagtgt |
| 485 | NM_001280 | CIRBP | cctgtgaaagtcggattcgaataaagggcc |
| 486 | NM_001295 | CCR1 | aacaatgatggacttcaattgtactcactg |
| 487 | NM_001312 | CRIP2 | tgtgctgtcaataaacggtttgaggattgc |
| 488 | NM_001313 | CRMP1 | tggcacttgtaactcaccgtgctgtcttct |
| 489 | NM_001337 | CX3CR1 | cttcaagatcatgtaccccaatttacttgc |
| 490 | NM_001344 | DAD1 | agatacgaacaacgcaaccgcacgcagcca |
| 491 | NM_001345 | DGKA | ccctggatgcagacgccctgtacaatcaag |
| 492 | NM_001398 | ECH1 | gggcgatgacagttgtttctatgccttctg |
| 493 | NM_001404 | EEF1G | ccaccaattcatgcgctatcaaatgtatta |
| 494 | NM_001428 | ENO1 | agagtcatgtgagcctcgtgtcatctccgg |
| 495 | NM_001436 | FBL | cagcatactaaccagacagcgacacgcaga |
| 496 | NM_001441 | FAAH | acataggccaaggcccaactaacagtcaag |
| 497 | NM_001456 | ELNA | agggagccatttggtggcgctgcttgtctt |
| 498 | NM_001459 | FLT3LG | aagccctggatcactcgccagaacttctcc |
| 499 | NM_001462 | FPRL1 | ctgaaaaggaattgagaagtaattcctctg |
| 500 | NM_001489 | NR6A1 | gacattaggtcagccactagatgtatttgt |
| 501 | NM_001513 | GSTZ1 | cccgcgctgaaatttggcgtgaattaaact |
| 502 | NM_001515 | GTF2H2 | gttatggatgtcagggggaattgaaagacc |
| 503 | NM_001540 | HSPB1 | cccttgccgttacttacgaaacacccaaaa |
| 504 | NM_001560 | IL13RA1 | ttttggtagtcttcaaccagggattgtttc |
| 505 | NM_001607 | ACAA1 | ccgctgccgtctttgaataccctgggaact |
| 506 | NM_001630 | ANXA8 | ccaaggacactgtgttataagaggcgtggg |
| 507 | NM_001640 | APEH | tctggctacgcacacacttgggcagctgaa |
| 508 | NM_001654 | ARAF | ccatgtggattttgggggtcccttttgtgt |
| 509 | NM_001664 | RHOA | ctcataccacttgtttctgccgtgctcatg |
| 510 | NM_001700 | AZU1 | gctgtaataaagaagccgatctctcctctg |
| 511 | NM_001712 | CEACAM1 | acacggactgtgcatactttccctcatcca |
| 512 | NM_001715 | BLK | tagtatttgggcatggcttacacctgtcat |
| 513 | NM_001716 | BLR1 | aagctgggtaatcgatgggggagtctgaag |
| 514 | NM_001728 | BSG | aatgctgtctggttgcgccatttttgtgct |
| 515 | NM_001734 | C1S | tggacaaatcgccagagatgtggtaagtga |
| 516 | NM_001743 | CALM2 | gttgttgttgaagtgtggagttgtaactct |
| 517 | NM_001747 | CAPG | aagacggcagaacgagtaagaacagacagg |
| 518 | NM_001752 | CAT | cccactttctatagcagattgtgtaacaat |
| 519 | NM_001753 | CAV1 | atgcatgtcctgtaaaggttacaagcctgc |
| 520 | NM_001759 | CCND2 | agctgccttttaagaggtcttatctgttca |
| 521 | NM_001767 | CD2 | agttacagggtgatgcttagagagggcttg |
| 522 | NM_001772 | CD33 | cactaaagatcacgtaccatatgattccat |
| 523 | NM_001774 | CD37 | ttcatgacgctctcgatattcctgtgcaga |
| 524 | NM_001776 | ENTPD1 | tctagaaaactccttaccatttgtcttagg |
| 525 | NM_001783 | CD79A | ctctttcctcgcgccgtgccgtttttacct |
| 526 | NM_001786 | CDC2 | cttatcttggctttcgagtctgagtttaaa |
| 527 | NM_001793 | CDH3 | agatgaagggtgaggacaatcgtgtatatg |
| 528 | NM_001794 | CDH4 | cataactgccaggttccgacttctgtccac |
| 529 | NM_001795 | CDH5 | ctgcaagggattgtagataacactgacttg |
| 530 | NM_001798 | CDK2 | ggtcatagtgggtggatttgttgccatgtg |
| 531 | NM_001830 | CLCN4 | tgttggtcttcaggaggggaagtttgctgt |
| 532 | NM_001838 | CCR7 | ttctgctttcgattcgttaagagagcaaca |
| 533 | NM_001839 | CNN3 | ccaatgtatcaatgtagagttgctctgttt |
| 534 | NM_001897 | CSPG4 | aataggataatatattcagggtgcagggtg |
| 535 | NM_001901 | CTGF | ccaaaagttacatgtttgcacctttctagt |
| 536 | NM_001905 | CTPS | cagactcgatgtgtactctaacttaagaaa |
| 537 | NM_001908 | CTSB | gccacaaattcataaccacttatccttcca |
| 538 | NM_001916 | CYC1 | acaacaagacaacgcacgaacaacgagcgg |
| 539 | NM_001923 | DDB1 | atgggtagcctgggtgtaaaacctggagat |
| 540 | NM_001928 | DF | ggagtcgatataaggcgtacggcagaatgc |
| 541 | NM_001939 | DRP2 | tgattcttctgaccctagaaatgttccctt |
| 542 | NM_001941 | DSC3 | ccaagactttactagtgccgataaactttc |
| 543 | NM_001952 | E2F6 | ctcattgggtaagtcatttaccactctgtg |
| 544 | NM_001964 | EGR1 | tattgcccatgggatatgtggtgtatatcc |
| 545 | NM_001993 | F3 | ggtacttaaagcttctatggttgacattgt |
| 546 | NM_001999 | FBN2 | accaaacctcatatgtgaaatggccaaagc |
| 547 | NM_002015 | FOXO1A | cagattccacgttcttgttccgatactctg |
| 548 | NM_002017 | FLI1 | tggaaatgcgtgtaacagtactgcaataat |
| 549 | NM_002023 | FMOD | catgagtggtggctaacctgaccaataaag |
| 550 | NM_002033 | FUT4 | ccttggtatttagtttggtgaatacttgca |
| 551 | NM_002046 | GAPD | cccacaaacacgaaacgggaatccattttt |
| 552 | NM_002049 | GATA1 | gaaggatggtattcagactcgaaaccgcaa |
| 553 | NM_002064 | GLRX | ccctacaccaagagtgtatctgtgaaagag |
| 554 | NM_002065 | GLUL | gtaaacaaactgtgtaactgcccaaagcag |
| 555 | NM_002067 | GNA11 | cctcttcacccatcaacgctgtgctttgcc |
| 556 | NM_002068 | GNA15 | accagaattctaggtgcgagtctcagctgt |
| 557 | NM_002076 | GNS | ccttcaaaaggaccctgttcactgctgcac |
| 558 | NM_002086 | GRB2 | atagcatttggtaggtagtgattaactgtg |
| 559 | NM_002090 | CXCL3 | cttacttctctcctgtcagttggtgctccc |
| 560 | NM_002094 | GSPT1 | agacagcaggttaatacctagatctcattt |
| 561 | NM_002102 | GYPE | ccatgcaaaactgtagatcaataaaacctc |
| 562 | NM_002107 | H3F3A | attttgggtagacgtaatcttcaccctttc |
| 563 | NM_002108 | HAL | ggaatctttggggattaaccaacctccttt |
| 564 | NM_002110 | HCK | aatccacaatctgacattctcaggaagccc |
| 565 | NM_002118 | HLA-DMB | gtgtgccatagaggacagcaactggtgatt |
| 566 | NM_002121 | HLA-DPB1 | caaatctgcctgataggacccatattccca |
| 567 | NM_002128 | HMGB1 | tgtgtacccgaggggtaatgattaaatgat |
| 568 | NM_002129 | HMGB2 | gcacagcaaacttgtaggaattagtatcaa |
| 569 | NM_002135 | NR4A1 | tttttaacacatagcgccgtgctgtaaata |
| 570 | NM_002141 | HOXA4 | gtgcatttagacttatgtggaactgtacaa |
| 571 | NM_002145 | HOXB2 | acacagaaaaattccgtttggtagactcct |
| 572 | NM_002162 | ICAM3 | tccagttctgtgcggcatgagaaaggggct |
| 573 | NM_002166 | ID2 | tgtgtttattgaatggtgattgcctgcttt |
| 574 | NM_002167 | ID3 | aggcacaagaacacgagacaacgcagacac |
| 575 | NM_002186 | LL9R | cacataagtgcatcttgcggggcagtgagt |
| 576 | NM_002193 | INHBB | gaaactggactcgtacgactctttttatat |
| 577 | NM_002198 | IRF1 | tacactaacatttcccccgagctcttggcc |
| 578 | NM_002199 | IRF2 | tatgcttgaatgccacgcggtacaaaggaa |
| 579 | NM_002222 | ITPR1 | gcctagttttgtacatggatctcattttac |
| 580 | NM_002224 | ITPR3 | tgagctgcgcttgcgtggctgtttcatgac |
| 581 | NM_002228 | JUN | gcttaccaaaggatagtgcgatgtttcagg |
| 582 | NM_002229 | JUNB | gaaacacgcacttagtctctaaagagttta |
| 583 | NM_002230 | JUP | tgcagggtcgttgtcaggttatgtgagaaa |
| 584 | NM_002248 | KCNN1 | accaaccgcgttgatggacacccactgtgt |
| 585 | NM_002276 | KRT19 | aacggagagaaggacacgacgacagagcag |
| 586 | NM_002295 | LAMR1 | acataagatgcctccgtgagaggctggtgg |
| 587 | NM_002312 | LIG4 | gatccatgcgatactatgtgtgcattattt |
| 588 | NM_002341 | LTB | agtcaagggagtagccggggtagtggctta |
| 589 | NM_002349 | LY75 | gaaaagcacggctgtatataatggtgatgt |
| 590 | NM_002351 | SH2D1A | agcatttaatatggatgccgtgggagtaca |
| 591 | NM_002353 | TACSTD2 | ggcgttgtggctctaagattaaacgacttt |
| 592 | NM_002356 | MARCKS | cctgcttaaggtgttctaattttctgtgag |
| 593 | NM_002379 | MATN1 | tcactcatcgcagttctccccacggctact |
| 594 | NM_002388 | MCM3 | tctttgcagtagcaccagttaaggtgaata |
| 595 | NM_002390 | ADAM11 | gatcgtctccaattcgaaaacaaccgtcct |
| 596 | NM_002392 | MDM2 | cctactttggtagtggaatagtgaatactt |
| 597 | NM_002393 | MDM4 | ccactaacttctgtaatgtaagattgagtc |
| 598 | NM_002398 | MEIS1 | gcgcatggctgaaatcctaggcgagaagaa |
| 599 | NM_002412 | MGMT | ccgctgtattaaaggaagtggcagtgtcct |
| 600 | NM_002414 | CD99 | ccctgccgccctaacaataggagatagaag |
| 601 | NM_002415 | MIF | tcaccccctttggttcattctcccgaatgt |
| 602 | NM_002417 | MKI67 | ctggttactgtatcccagtgaccagccaca |
| 603 | NM_002419 | MAP3K11 | ttgaccggaggagaaacgtcttcgaggtcg |
| 604 | NM_002421 | MMP1 | ggcaagggataactcttctaacacaaaata |
| 605 | NM_002422 | MMP3 | agagaaggggaagcactcgtgtgcaacaga |
| 606 | NM_002436 | MPP1 | tctataatgccaaaatgacacatctgtgca |
| 607 | NM_002441 | MSH5 | tgtcctcattctcccctatatgacatgcaa |
| 608 | NM_002444 | MSN | atgtcctatattggctaaattgaaacctgg |
| 609 | NM_002447 | MST1R | cccttgaaccaataaaggaacaaatgacta |
| 610 | NM_002460 | IRF4 | gtaagttatgttttacatgccccgtttttg |
| 611 | NM_002462 | MX1 | ttcagccctcagaatcgctccaccttgcag |
| 612 | NM_002466 | MYBL2 | gctcctggtgctaacaacaaagttccactt |
| 613 | NM_002475 | MLC1SA | tgcactatcccacacacgtactcaccacgc |
| 614 | NM_002483 | CEACAM6 | tttccttgttccaatttgacaaaacccact |
| 615 | NM_002512 | NME2 | acagcgcacgacagcacgaagcggaaaaac |
| 616 | NM_002514 | NOV | ttaatgccaccacacgtgttccgcttctct |
| 617 | NM_002527 | NTF3 | aaaacttgttttgtgatccggctctcagga |
| 618 | NM_002530 | NTRK3 | gttgaacatcaaggagatctacaaaatcct |
| 619 | NM_002572 | PAFAH1B2 | atgcccggctcttaggaattttgtctgttt |
| 620 | NM_002576 | PAK1 | tcatgctctgaggtactactgcctctgcag |
| 621 | NM_002585 | PBX1 | tcagcctggttttcgtcatcttccctgccc |
| 622 | NM_002592 | PCNA | aagtgaacacacacgaccaacagcagcgcg |
| 623 | NM_002600 | PDE4B | tcttctgataacacttgttaggcctcttac |
| 624 | NM_002608 | PDGFB | cacgtgaacttgtgcgtgtctctggcagtg |
| 625 | NM_002609 | PDGFRB | ggactcacgttaactcacatttatacagca |
| 626 | NM_002616 | PER1 | gctgtaccctctgccgatggcatttgtgtt |
| 627 | NM_002619 | PF4 | cattcttcagcgtggctatcagttgggcag |
| 628 | NM_002621 | PFC | tcaaccccgtgaggtgagtattaatattac |
| 629 | NM_002648 | PIM1 | ggcattgctgactgtgtatataggataatt |
| 630 | NM_002658 | PLAU | ccttttagcctagttcatccaatcctcact |
| 631 | NM_002659 | PLAUR | cactctcctctggacctaaacctgaaatcc |
| 632 | NM_002687 | PNN | tatctgttggccattactatacatgtgcaa |
| 633 | NM_002704 | PPBP | gaacccattgcaaccaagtcgaagtgatag |
| 634 | NM_002729 | HHEX | aataaagccgtccgtgggacgactgacctc |
| 635 | NM_002730 | PRKACA | tgcgaaggacgagacttcctcttgaacagt |
| 636 | NM_002744 | PRKCZ | gttcatctttcgtcctgtgtctgactttct |
| 637 | NM_002753 | MAPK10 | tatatggggggagtgagctctctcagaccc |
| 638 | NM_002758 | MAP2K6 | atagatcccctgagaggtatctgagaagaa |
| 639 | NM_002791 | PSMA6 | aagacgagacgcaaacgagccaagcagagg |
| 640 | NM_002800 | PSMB9 | tagcatataagccaggcatgtctcccgagg |
| 641 | NM_002814 | PSMD10 | cacattttaagttggatggtgtgctctaaa |
| 642 | NM_002827 | PTPN1 | atgtgacaacaattgctaaagcaaaccttg |
| 643 | NM_002831 | PTPN6 | tctccctgaccctgtatatagcccagccag |
| 644 | NM_002835 | PTPN12 | cagtaagtgccttggaacaatattgaattc |
| 645 | NM_002844 | PTPRK | tttctagattgccagctcatgacatggtgc |
| 646 | NM_002856 | PVRL2 | actggacttctcccgtctctagggctgcat |
| 647 | NM_002868 | RAB5B | acagaatgcgactgctgatttaccgatgta |
| 648 | NM_002870 | RAB13 | aggctagctcggcactacactagggaattt |
| 649 | NM_002880 | RAF1 | tttcttaggggacacgtcctcctttcacag |
| 650 | NM_002887 | RARS | tgtccaaaggatgtaatccttcataggttt |
| 651 | NM_002893 | RBBP7 | acaagtagcagggtattgcctttgattcaa |
| 652 | NM_002901 | RCN1 | gcttataccatggaatgaggacaaggtgat |
| 653 | NM_002908 | REL | gcaattgagtgactcctttccatatgaatt |
| 654 | NM_002913 | RFC1 | aggaactggtagtcttgtttgggtgatgag |
| 655 | NM_002934 | RNASE2 | tgacctgtcctagtaacaaaactcgcaaaa |
| 656 | NM_002935 | RNASE3 | acgggattctccacggtatcctgtggttcc |
| 657 | NM_002936 | RNASEHI | cattagttgtctgtcaaggaaggtgagtgg |
| 658 | NM_002953 | RPS6KA1 | cagctccttttccgttctgttctgctggga |
| 659 | NM_002960 | S100A3 | gcgcagaagtgtgaccgactgaatcataaa |
| 660 | NM_002961 | S100A4 | ctcgcgttctgatgacacgtctactaaaac |
| 661 | NM_002971 | SATB1 | agaaatgttctgactaggagtggttattga |
| 662 | NM_002972 | SBF1 | cagagtctatataaagagagaactaacgcc |
| 663 | NM_002975 | CLEC11A | atgttggcgtacacgttgcaggctgcgtga |
| 664 | NM_002983 | CCL3 | cctcttctatggactggttgttgccaaaca |
| 665 | NM_002984 | CCL4 | gttatgacgaaccatccgcccgccaagctt |
| 666 | NM_002985 | CCL5 | attaccactcaaacgcgaaacacaatcaaa |
| 667 | NM_003002 | SDHD | aacgatagagacagaacgcatgcgcgcagg |
| 668 | NM_003005 | SELP | tcaagctagaaggccacatgtcaccgtgga |
| 669 | NM_003006 | SELPLG | gtactgaagagtgacggacttctgaggctg |
| 670 | NM_003009 | SEPW1 | agcgagacaaacgcgagcataggacaccca |
| 671 | NM_003025 | SH3GL1 | acgtctccagcacacctgaagaactggagg |
| 672 | NM_003038 | SLC1A4 | gctgttctctttctaacatgttgtggtaaa |
| 673 | NM_003039 | SLC2A5 | tattaccggcctcgtcggtgtggtctgtct |
| 674 | NM_003058 | SLC22A2 | tcatgaggtccttccatatttagattgggt |
| 675 | NM_003072 | SMARCA4 | gtgcgtcaccgtccactcctcctactgtat |
| 676 | NM_003073 | SMARCB1 | ttgtcaccaccatcgcatacagcatccggg |
| 677 | NM_003100 | SNX2 | aagtggactgtggcatgacattctgcaata |
| 678 | NM_003113 | SP100 | catagtgctaaaagcagaactgccattcca |
| 679 | NM_003118 | SPARC | ccaaaatcaagagtgagatgtagaaagttg |
| 680 | NM_003120 | SPI1 | gaaggggcggttctcctcgaccttagatga |
| 681 | NM_003121 | SPIB | gttcaaagagagaattatatgctggcgcgg |
| 682 | NM_003123 | SPN | cttcttgacgcttttcataatgtgcaaagt |
| 683 | NM_003127 | SPTAN1 | tgccctcattccgacttcagaaaatcgaag |
| 684 | NM_003128 | SPTBN1 | tctctgtgcctaatgttcctcaatgtggtt |
| 685 | NM_003133 | SRP9 | gcatggtaagttcccttagctatatgaatt |
| 686 | NM_003159 | CDKL5 | gacataccatgagaatgcggcactgacggg |
| 687 | NM_003189 | TAL1 | gtgaggatgtatcgggaagatttctaaata |
| 688 | NM_003200 | TCF3 | gacctgtcctgtatataacagcactgtagc |
| 689 | NM_003222 | TFAP2C | cccaaataattgttgtgtgtatgatacgtg |
| 690 | NM_003225 | TFF1 | cgccactacgcctctactacacgaacacaa |
| 691 | NM_003234 | TFRC | gaatgaaatatcagactagtgacaagctcc |
| 692 | NM_003262 | TLOC1 | tcaaaggccaatgatacttcgcataattca |
| 693 | NM_003288 | TPD52L2 | cccatgcccactctttaggcacaaatgcag |
| 694 | NM_003302 | TRIP6 | tcctttgattgatcactctccctgacatcc |
| 695 | NM_003311 | PHLDA2 | aaatgaaattgcggccattgatctctaacc |
| 696 | NM_003315 | DNAJC7 | aaagggaaccaatgccgaagaccgagggca |
| 697 | NM_003318 | TTK | attttactcttggaatagtgggtggatagc |
| 698 | NM_003352 | SUMO1 | gagaggaaacgagtagggacaaacacgagg |
| 699 | NM_003361 | UMOD | aatgggacttgtgacggtgtacctgaggcc |
| 700 | NM_003383 | VLDLR | aggacaaaacgggttactaagatgaaattg |
| 701 | NM_003390 | WEE1 | ttcttggttgagattggttggttgagactc |
| 702 | NM_003395 | WNT9A | tccgagtctccgtgcgtgcaatgatttctc |
| 703 | NM_003403 | YY1 | gtgctcagttgtagaatgtattgtaccttt |
| 704 | NM_003415 | ZNF268 | tgtatcggaagcatataccttggagggacc |
| 705 | NM_003425 | ZNF45 | tcagtttctggctagcaactagaagttttg |
| 706 | NM_003427 | ZNF76 | ggtggcacatggatgaatgagtatttaata |
| 707 | NM_003453 | ZNF198 | ccttacaaggttatgtagagagataccatc |
| 708 | NM_003467 | CXCR4 | cctacagtgtacagtcttgtattaagttgt |
| 709 | NM_003480 | MFAP5 | tgaccctcctactccacattgcaacatttc |
| 710 | NM_003549 | HYAL3 | atagactgccccgacagtctacaagcctca |
| 711 | NM_003582 | DYRK3 | gtggacagagatatgcccagagatgcatat |
| 712 | NM_003627 | SLC43A1 | tacccctgaggtcccatgtgccatgagtgt |
| 713 | NM_003636 | KCNAB2 | agactcagaccgcgacacagccaccgtatt |
| 714 | NM_003645 | SLC27A2 | gaaatttgcatacccgtaaagggagacttt |
| 715 | NM_003692 | TMEFF1 | cagattggaataaagcctattctaccagtt |
| 716 | NM_003701 | TNFSF11 | caccaggtgcctttcaaatttagaaactaa |
| 717 | NM_003707 | RUVBL1 | tgaaagaacccttccttacctggtgtgttt |
| 718 | NM_003739 | AKR1C3 | acttctcccgccgtattggttgcggacctt |
| 719 | NM_003746 | DNCL1 | aaggagcgaccgaagacaagcgaacgagac |
| 720 | NM_003753 | EIF3S7 | cttagtggaatgtgtgtctaacttgctctc |
| 721 | NM_003801 | GPAA1 | atgttccgcaagctcaaccacctcctggag |
| 722 | NM_003805 | CRADD | tcctcccctgtaatacttagcaggccagtt |
| 723 | NM_003821 | RIPK2 | gtccccatttttaacctcagccttccctac |
| 724 | NM_003824 | FADD | aggcagcgggatctcgtatctttaaaaagc |
| 725 | NM_003842 | TNFRSF10B | ctgccctgtcaaaggtccctatttgaaatg |
| 726 | NM_003852 | TIF1 | gtaggttagatgctattaagaaggcactta |
| 727 | NM_003874 | CD84 | ttggtccttctatcactaaaactcatctca |
| 728 | NM_003877 | SOCS2 | tttctgaatgaccggaagttcttctgatct |
| 729 | NM_003879 | CFLAR | acatggtaaacgctgtccctagtaaaaatg |
| 730 | NM_003884 | PCAF | gctttgtaccggatctgctgaagcatctgt |
| 731 | NM_003900 | SQSTM1 | tggatgggactccatagctccttcccagga |
| 732 | NM_003902 | FUBP1 | agggccaataataagaagtggacaatacag |
| 733 | NM_003914 | CCNA1 | atggaagcacttccagaacttcacctccat |
| 734 | NM_003954 | MAP3K14 | agactccgtgggagaagctcattcccacat |
| 735 | NM_003955 | SOCS3 | gtattctgtgtcaggtattgggctggacag |
| 736 | NM_003974 | DOK2 | cctgcacctttcagatttcttggtggcatt |
| 737 | NM_003998 | NFKB1 | attaaaggtatcacggtcgccacctggcat |
| 738 | NM_004039 | ANXA2 | acacagacggaagggacaaggagcggaaca |
| 739 | NM_004047 | ATP6V0B | ttgctgcgtgttgatttggaggcactgcag |
| 740 | NM_004064 | CDKN1B | cttaaaaggttgcatactgagccaagtata |
| 741 | NM_004067 | CHN2 | tattttacgtggagcatcattgtgtgactg |
| 742 | NM_004077 | CS | taaacccctgggtaactgtcaagtcatgat |
| 743 | NM_004083 | DDIT3 | acatacgcagggggaaggcttggagtagac |
| 744 | NM_004088 | DNTT | gttgtcactggtggctcattcagggaagct |
| 745 | NM_004093 | EFNB2 | ttgctgcatatttgtgccgtaattatgtac |
| 746 | NM_004099 | STOM | cttgggagggacgcatagaaggagctctag |
| 747 | NM_004104 | FASN | taactgtcagtgtacacgtctggaccccgt |
| 748 | NM_004111 | FEN1 | gaaactcgaacttgctatgtaatttgtgtc |
| 749 | NM_004119 | FLT3 | tggagcattgatctgcatccaaggccttct |
| 750 | NM_004177 | STX3A | cttacagtttggaatacaacatgtgaaggt |
| 751 | NM_004208 | PDCD8 | gagtgtgaatgatcaagtcctttgtgaata |
| 752 | NM_004221 | NK4 | aaggacagaaccacgacacgacacagagaa |
| 753 | NM_004235 | KLF4 | tttttatgcagacagtctgttatgcactgt |
| 754 | NM_004310 | RHOH | gaaatctagccagtgtgtagacaatgttga |
| 755 | NM_004313 | ARRB2 | taaaacagagatattaggagctccatcact |
| 756 | NM_004320 | ATP2A1 | tccccaccccgatagtgacacatcttcagg |
| 757 | NM_004324 | BAX | acgcaaggactagcggagacaagacagagg |
| 758 | NM_004335 | BST2 | tgtttgtaaaaagggccagacaaaggggca |
| 759 | NM_004336 | BUB1 | gctcttagaatgtaagcgttcacgaaaata |
| 760 | NM_004343 | CALR | ctggttcggatttggggtggattttggttt |
| 761 | NM_004355 | CD74 | acagggagaagggataaccctacacccaga |
| 762 | NM_004374 | COX6C | ggctattcagagtggtcagggagcttagag |
| 763 | NM_004380 | CREBBP | ctttaagatcccctgtaagaatgtttaagg |
| 764 | NM_004385 | CSPG2 | aaggactccaatgtcgaactcttctttgct |
| 765 | NM_004413 | DPEP1 | caagtatccagacctgatcgctgagctgct |
| 766 | NM_004425 | ECM1 | ctgaacactcattacactaaacacctcttg |
| 767 | NM_004430 | EGR3 | gacaatcagccggcctataaatctccttat |
| 768 | NM_004448 | ERBB2 | ttcttgtatcccttttacagtcaaagtcca |
| 769 | NM_004449 | ERG | tcatggatagctgcaatttctcactttaca |
| 770 | NM_004454 | ETV5 | catcctgccaagctgcgttatattctgtac |
| 771 | NM_004475 | FLOT2 | gttggggacagaaaagtgtgtagaccttct |
| 772 | NM_004494 | HDGF | gtcaccagatctgatttgtaacccactgag |
| 773 | NM_004501 | HNRPU | gtttctgacaataactgggaaggttttttc |
| 774 | NM_004508 | IDI1 | tcaacccccaactgaggaccactgtcctac |
| 775 | NM_004513 | IL16 | catgaaactccaccttgcggggataaagag |
| 776 | NM_004526 | MCM2 | cactctcttatttgtgcattcggtttggtt |
| 777 | NM_004530 | MMP2 | agcatgtccctaccgagtctcttctccact |
| 778 | NM_004557 | NOTCH4 | ctgctatcgctatttaagaaccctaaatct |
| 779 | NM_004583 | RAB5C | tgtgcctgtctacctgcgcgtttgctgtac |
| 780 | NM_004615 | TM4SF2 | cccacagtatattggttattggtcctgaag |
| 781 | NM_004624 | VIPR1 | tgcaacaggcttgtgcaacaataaatgttg |
| 782 | NM_004633 | IL1R2 | gaaaagcagatggtctgactgtgctatggc |
| 783 | NM_004642 | CDK2AP1 | gatctcagttctgcgtttattgtaagttga |
| 784 | NM_004688 | NMI | acaacaattgagacacaagcggtccctctc |
| 785 | NM_004689 | MTA1 | ggcttgcttctatgggctttgcaccaccct |
| 786 | NM_004706 | ARHGEF1 | catcactgagactgccggatccctgaaagt |
| 787 | NM_004725 | BUB3 | ccctagtaagcccagttgctgtatctgaac |
| 788 | NM_004815 | PARG1 | tagttctatgtgatgtaaaggtctagggag |
| 789 | NM_004827 | ABCG2 | gaataggtaggttagtagcccttcagtctt |
| 790 | NM_004867 | ITM2A | gagtttaggttgtatgaattctacaaccct |
| 791 | NM_004924 | ACTN4 | atgaagacatagccgattctctgcccgggc |
| 792 | NM_004936 | CDKN2B | cctgttaagttcgtatctaaacctaagaca |
| 793 | NM_004938 | DAPK1 | ggtttcataacttcctgtacttgaagtcta |
| 794 | NM_004941 | DHX8 | accagcaggtggtctatatccatccttcca |
| 795 | NM_004972 | JAK2 | gctcattaagaagtgcagcaggttaagaat |
| 796 | NM_004994 | MMP9 | ctacgtgacctatgacatcctgcagtgccc |
| 797 | NM_004997 | MYBPH | accaggcctacagtcaaactccagagatgc |
| 798 | NM_005012 | ROR1 | catggtggagcgtgacgattgtcccagcta |
| 799 | NM_005080 | XBP1 | ctagtgtagcttctgaaaggtgctttctcc |
| 800 | NM_005084 | PLA2G7 | catgttacagaactcttcaggaatagagaa |
| 801 | NM_005085 | NUP214 | tccctcccactattaaacagtctgtttccg |
| 802 | NM_005098 | MSC | aagtgatcctgcgatagggaacgcgccctt |
| 803 | NM_005104 | BRD2 | ctccccatttggtcccctggactgtctttg |
| 804 | NM_005123 | NR1H4 | cacaactgtaaatattgggctagatagaac |
| 805 | NM_005157 | ABL1 | ttctaagtggcgtgtgcatagcgtcctgcc |
| 806 | NM_005161 | AGTRL1 | ctcaaagcatccagtgaacactggaagagg |
| 807 | NM_005178 | BCL3 | gtaacgggcacggatcacgatgtaaattat |
| 808 | NM_005187 | CBFA2T3 | catgatggtaataaacgagatgtttgccaa |
| 809 | MM_005192 | CDKN3 | aacttctctccgccccggttttctttcccc |
| 810 | NM_005197 | CHES1 | ttcagcaaacaagtgctagcttcactgacc |
| 811 | NM_005215 | DCC | gcataccaattacccataaacagcacacct |
| 812 | NM_005225 | E2F1 | aggcctgggtgatttatttattgggaaagt |
| 813 | NM_005230 | ELK3 | gggcaatatgaaagcatatatcacgttttg |
| 814 | NM_005239 | ETS2 | ggggttcagcctaacagttatggaaactac |
| 815 | NM_005245 | FAT | ttattggaaagtgtaacggggaccttctgc |
| 816 | NM_005246 | FER | gtcctccagcagagtaacattattgttctc |
| 817 | NM_005248 | FGR | acaccacctagggcaacctacttgttttac |
| 818 | NM_005252 | FOS | attgttgaggtggtctgaatgttctgacat |
| 819 | NM_005263 | GFI1 | ttgaccctccatgtgtaccatagagggggg |
| 820 | NM_005317 | GZMM | acagggagggaccaataaatcataatgaag |
| 821 | NM_005318 | H1F0 | gggctagtacttagtttcacacccgggagc |
| 822 | NM_005334 | HCFC1 | taccatagtgcgatgtcgttttgtgctatt |
| 823 | NM_005335 | HCLS1 | acccgggaaagtacgtctagattgtgtggt |
| 824 | NM_005339 | HIP2 | gaaggttggggtatttgtcttgagaattaa |
| 825 | NM_005340 | HINT1 | aggcaatgattaagttaggcaatttccagt |
| 826 | NM_005356 | LCK | caggactttatctaatacctctgtgtgctc |
| 827 | NM_005375 | MYB | atgacggtgtacttactgccttgtagcaaa |
| 828 | NM_005385 | NKTR | cctctgctgcagcgttcaacttgtgtgttt |
| 829 | NM_005424 | TIE1 | aacatgccctgttcagctactcccactccc |
| 830 | NM_005427 | TP73 | tttttaacaggattggggtgtccaaactgc |
| 831 | NM_005439 | MLF2 | gatcccatcgtgaccgacggtgtttgtgta |
| 832 | NM_005494 | DNAJB6 | aaaactgttggtgaggtagtgtttcagtta |
| 833 | NM_005504 | BCAT1 | gatcatccgcaggctatgttaaaaggattt |
| 834 | NM_005521 | TLX1 | atgggcatctatgggagagtgtcaaccaga |
| 835 | NM_005529 | HSPG2 | gtcctagaagggaccctcctgtggtctttg |
| 836 | NM_005533 | IFI35 | ccgaatcttctgtgttcgttcttctttgct |
| 837 | NM_005556 | KRT7 | gatgaattccactggtggcagtagcagtgg |
| 838 | NM_005563 | STMN1 | ttatttaaagggaccttattttctgcccct |
| 839 | NM_005566 | LDHA | ttttggactcctatcccgttatgtctgggc |
| 840 | NM_005572 | LMNA | agcctgcgtacggctctcatcaactccact |
| 841 | NM_005583 | LYL1 | ttaacccctttggtactctcctgcatctcc |
| 842 | NM_005589 | ALDH6A1 | gcttatctgaaagtagtgcttaaacctgat |
| 843 | NM_005608 | PTPRCAP | tcacgcgtacctctacttatcaactaacgt |
| 844 | NM_005621 | S100A12 | ggagtaagagttaataaacacactcacgaa |
| 845 | NM_005651 | TDO2 | gtgacaagactaagcattaagatgagaaag |
| 846 | NM_005654 | NR2F1 | aggatcagatctgtgagcacgttggcgagg |
| 847 | NM_005671 | D8S2298E | actttcgaatcacaccataccttgattgag |
| 848 | NM_005687 | FARSLB | aagctgggtctcatgtttgagacaagttcc |
| 849 | NM_005730 | CTDSP2 | tcccccttcaagatgccatttggagggtag |
| 850 | NM_005731 | ARPC2 | tcctcgcgcccgtgcaggtgacaaataaca |
| 851 | NM_005738 | ARL4A | ccctttgtagcctaagtacgtaaacaaatt |
| 852 | NM_005761 | PLXNC1 | gtttgcacataggttccactttgggcactg |
| 853 | NM_005767 | P2RY5 | tcactgggacagaactttcaagttccttca |
| 854 | NM_005781 | TNK2 | agcggagaaagggtgccagaccattctctg |
| 855 | NM_005794 | DHRS2 | ggctgatccaattaacatgtggggttcttg |
| 856 | NM_005808 | CTDSPL | caatcaaaaggtttgcaatgatttccttcc |
| 857 | NM_005809 | PRDX2 | atatatatcgcgacatcaccggcccgaatc |
| 858 | NM_005826 | HNRPR | cccaatgctttctgatagtaggagataatt |
| 859 | NM_005835 | SLC17A2 | cagcaccgatcagaactgtaaagctataat |
| 860 | NM_005845 | ABCC4 | gacagtcatataaacgtcctgtttctgttt |
| 861 | NM_005870 | SAP18 | ggatgtgctattgtatgattacgaatagtc |
| 862 | NM_005895 | GOLGA3 | aatgttgtacgtgaagattctaggagggga |
| 863 | NM_005900 | SMAD1 | gctctcgtacattgagtacttttattccaa |
| 864 | NM_005902 | SMAD3 | gtctaccagttgacccgaatgtgcaccatc |
| 865 | NM_005907 | MAN1A1 | caaccccatggattaggctgaaacatatga |
| 866 | NM_005911 | MAT2A | cttaattgccacctctaacagcaccaaatc |
| 867 | NM_005923 | MAP3K5 | gcttgttctaaagctatccattgtgtcata |
| 868 | NM_005933 | MLL | ggccctaggggttccactagtgtctgcttt |
| 869 | NM_005936 | MLLT4 | cccatacacaagtggatttggccatctttg |
| 870 | NM_005944 | CD200 | tgttctaaagatattgttccagctagtgga |
| 871 | NM_005954 | MT3 | tgacgcgtttccgcgtattgtcccctataa |
| 872 | NM_005956 | MTHFD1 | ctgggagttaggaagtataagtaagccaag |
| 873 | NM_005957 | MTHFR | gcaactagccaaggctagttcctttcaatt |
| 874 | NM_005965 | MYLK | aaaggataccacagtgcatgctgtttgttc |
| 875 | NM_006005 | WFS1 | ggtgaatgtgtgaaggtcttgcctgaatcc |
| 876 | NM_006017 | PROM1 | actggtcggacaaaatttaacgttgatgta |
| 877 | NM_006038 | SPATA2 | gtcacagactggaatacgaaatactacagc |
| 878 | NM_006060 | ZNFN1A1 | ggcatttggaaacgggaataaacaaaattg |
| 879 | NM_006079 | CITED2 | ggccaaactgttctggatcaggaaagtcat |
| 880 | NM_006082 | K-ALPHA-1 | gaaggagaggaatactaattatccattcct |
| 881 | NM_006084 | ISGF3G | tcatctgtaagggactaggaaattccaaat |
| 882 | NM_006098 | GNB2L1 | tcggcacacgctagaagtttatggcagagc |
| 883 | NM_006101 | KNTC2 | ggagcagattgctaaagttgatagagaata |
| 884 | NM_006113 | VAV3 | ctgcagatttgtgtagagttctaataccaa |
| 885 | NM_006120 | HLA-DMA | gtgtcccttccccttatgattttactttcc |
| 886 | NM_006142 | SFN | cagtgcaagaccgagattgagggaaagcat |
| 887 | NM_006152 | LRMP | ttatctccccaactaaaatacaatggggaa |
| 888 | NM_006184 | NUCB1 | ttccatgttccggttccatccaaatacact |
| 889 | NM_006185 | NUMA1 | ttaagggccggggatggatcttttctaaat |
| 890 | NM_006187 | OAS3 | cagcatattcataactagagccatatcaca |
| 891 | NM_006190 | ORC2L | cagcttacagtcatcacataccccctgaaa |
| 892 | NM_006191 | PA2G4 | aggtttgtgccccactacagaacagggcta |
| 893 | NM_006196 | PCBP1 | atgatccaactgtgtaatttctggtcagtg |
| 894 | NM_006209 | ENPP2 | acatttagtcctgtactgtatggaaacact |
| 895 | NM_006216 | SERPINE2 | ctcctgtcttgctagacaaggttgctgttg |
| 896 | NM_006230 | POLD2 | tattagtttcctgtaggcgctgtaacaaag |
| 897 | NM_006231 | POLE | agcactacggcatgtcgtacctcctggaga |
| 898 | NM_006235 | POU2AF1 | agaatccatgtagaagtcatggacttttct |
| 899 | NM_006255 | PRKCH | ctcatgtcagtgaccagatttgtggcttat |
| 900 | NM_006263 | PSME1 | gacgctcccttgtcgttcagtatgtcggag |
| 901 | NM_006268 | DPF2 | cctaactcactgtcgctttggagttgaggt |
| 902 | NM_006273 | CCL7 | gtagttgaagtattaatcccaactggctga |
| 903 | NM_006286 | TFDP2 | attcctatgaccaaattgcttgcctacagt |
| 904 | NM_006299 | ZNF193 | tattacccctatatgcatcaaagctgcaac |
| 905 | NM_006301 | MAP3K12 | tactgtaacccctgtgaaaaaccttgaaat |
| 906 | NM_006302 | GCS1 | ttggataccagtaattcaaaccttcctcat |
| 907 | NM_006325 | RAN | tattgcgaattgcgtcgaacctcccctccc |
| 908 | NM_006329 | FBLN5 | ttttacccaattggattggaatgcagaggt |
| 909 | NM_006343 | MERTK | ttttgtttgctgacgactcctcagaaggct |
| 910 | NM_006354 | TADA3L | caggcatctgtgactgaccgattgatttga |
| 911 | NM_006355 | TRIM38 | tccagtctgctaggaccaattaccttgaaa |
| 912 | NM_006362 | NXF1 | tgtacttaaccccaggacctccttactttt |
| 913 | NM_006427 | SIVA | taggactttgacgtttgaatggtggggggt |
| 914 | NM_006472 | TXNIP | ttggtcagtcactctcagccatagcacttt |
| 915 | NM_006495 | EVI2B | gatcaagatcttaatgaatccctgccacct |
| 916 | NM_006509 | RELB | gtggggggtaggttggttgttcagagtctt |
| 917 | NM_006522 | WNT6 | atccgcgcgttggtgcaactgcacaacaac |
| 918 | NM_006534 | NCOA3 | aaccacatgaaatgacttatgggggatggt |
| 919 | NM_006559 | KHDRBS1 | ctacatgtgtaagtctgcctaaataggtag |
| 920 | NM_006565 | CTCF | ttaggacgccaacatgagacctgtaataaa |
| 921 | NM_006579 | EBP | gggttgctctagtcagggatgttacagttg |
| 922 | NM_006591 | POLD3 | aattatgctggggtctcgactaaaactgaa |
| 923 | NM_006602 | TCFL5 | gaatgtattctgatcggatcgtttatggtt |
| 924 | NM_006676 | USP20 | atgtctgtgctgtctagtttgtgttcaaaa |
| 925 | NM_006739 | MCM5 | gtgcttttggcatccgttaataataaagcc |
| 926 | NM_006747 | SIPA1 | tggagaccctaagggaactccaaaatctga |
| 927 | NM_006748 | SLA | ggatgctgggattcttgcctgtatgaatgc |
| 928 | NM_006759 | UGP2 | gattgtgtgaactattcatctggctcttgg |
| 929 | NM_006762 | LAPTM5 | ttcagtcaaagcaggcaacccccttgtggg |
| 930 | NM_006769 | LMO4 | cagagacatttgctctagtatggtgtattt |
| 931 | NM_006803 | AP3M2 | aataactgtcctgtcacatgtgcagcaggc |
| 932 | NM_006820 | IFI44L | gaagcggtgggctaagataggtcctactgc |
| 933 | NM_006825 | CKAP4 | ctctgagctgtattgttctttaatggctgt |
| 934 | NM_006845 | KIF2C | ctaaaccttctacgcctttgggccgagcac |
| 935 | NM_006857 | RY1 | cctcccatgacttgccattataattaaaac |
| 936 | NM_006875 | PIM2 | accggacaccaccagacactaggatgggat |
| 937 | NM_006904 | PRKDC | tgttagggatagtactaagcatttcagttc |
| 938 | NM_006910 | RBBP6 | ctgaattcattacaaccctgttatgtcact |
| 939 | NM_006931 | SLC2A3 | gcagtaaggcagggtcccatttctcactga |
| 940 | NM_006932 | SMTN | cccatgatcatcatgtcgtccacctccacc |
| 941 | NM_006937 | SUMO2 | gcacttatcataagtaaatactccctaacc |
| 942 | NM_006938 | SNRPD1 | cagtgcctcctttattaaggggttctttga |
| 943 | NM_006963 | ZNF22 | tgccaagtgtcagactctaatgagccctca |
| 944 | NM_006981 | NR4A3 | tgccttctgtgggcggaaatcaggaagcca |
| 945 | NM_006994 | BTN3A3 | gagttgacatccctattgactctttcccag |
| 946 | NM_007022 | CYB561D2 | tacctataccgcaagaggatccaaccatga |
| 947 | NM_007063 | TBC1D8 | cactgtttcaatgctggtaattgaaaccat |
| 948 | NM_007065 | CDC37 | ctatttggcaaacagcaatgatcttccaat |
| 949 | NM_007079 | PTP4A3 | caggtgctccggacacccgaaggcaataaa |
| 950 | NM_007111 | TFDP1 | gaatgcaactttctaaacagccccaagcaa |
| 951 | NM_007117 | TRH | atcacagtagcgctcatggctcgtaggttt |
| 952 | NM_012092 | ICOS | tagtttacaagtgagacccgatatgtcatt |
| 953 | NM_012120 | CD2AP | tttttgtttgtgaccactaagcttctgtct |
| 954 | NM_012207 | HNRPH3 | cagcatctggtctactagactttcttacag |
| 955 | NM_012223 | MYO1B | aaggtgattggtacagggtgcctattttag |
| 956 | NM_012267 | HSPBP1 | gaagaccagaagcacgacagacgagacgag |
| 957 | NM_012317 | LDOC1 | acgttcgaggcgggcgccctcattatttgt |
| 958 | NM_012323 | MAFF | tacagcaagcgagttatcgtcttctgtatt |
| 959 | NM_012334 | MYO10 | tgtccctttgtgtacaaccatgcaaaactg |
| 960 | NM_012384 | GMEB2 | gcaagtatctgcctggtactttgatttaaa |
| 961 | NM_012417 | PITPNC1 | ttaaacatgtaacagttgcagattggctca |
| 962 | NM_012423 | RPL13A | ggcatctgttggactttccacctggtcata |
| 963 | NM_013282 | UHRF1 | agcactgcaaggacgacgtgaacagactct |
| 964 | NM_013314 | BLNK | ggtgaggttgagttatcatgctactaatat |
| 965 | NM_013416 | NCF4 | cttcacctcgatgacgaaaacctaaggagg |
| 966 | NM_014005 | PCDHA6 | attgaatgttctggttcaaggctccacctc |
| 967 | NM_014207 | CD5 | ctctttcatctatagtttggccaccataca |
| 968 | NM_014232 | VAMP2 | gtgttctagaccccccatattatcccagtg |
| 969 | NM_014244 | ADAMTS2 | aatattgtccccccctaccttaagaagata |
| 970 | NM_014298 | QPRT | aatggggcacatttggcactagcttgagcc |
| 971 | NM_014345 | ZNF318 | cacaaacccatatgtatcctttcctcaacc |
| 972 | NM_014365 | HSPB8 | ggtattgtgtatatgggcgggacgtgtgtg |
| 973 | NM_014575 | SCHIP1 | ctgtctctcccaacgtgactatgaattccc |
| 974 | NM_014595 | NT5C | cttacctactcccgtctagaaaatgctgaa |
| 975 | NM_014614 | PSME4 | ttatcttgttacaaaaactgagatgtgggg |
| 976 | NM_014624 | S100A6 | gatataggcatcgcacctctatcatcgtca |
| 977 | NM_014667 | VGLL4 | tgtcagtgaacaagggcgtcactctgggag |
| 978 | NM_014707 | HDAC9 | taatgaaggcactgcttatttgtagtcacc |
| 979 | NM_014724 | ZNF305 | acaagaccgtgtaggtgtcccttcagagag |
| 980 | NM_014734 | KIAA0247 | caggatgtcactcaatcagtttgggtttgc |
| 981 | NM_014735 | PHF16 | catgcctttcaattctgagtgggaggaaaa |
| 982 | NM_014780 | CUL7 | taccttccggtagccctagacttggggtca |
| 983 | NM_014792 | KIAA0125 | gacattgagaattggtacctacaacagtct |
| 984 | NM_014824 | FCHSD2 | gatcacttcagtgacaatacccattaatga |
| 985 | NM_014890 | DOC1 | cacacctcttcctcgacaatcacaaattac |
| 986 | NM_014899 | RHOBTB3 | agtataatattgggaccccataccgttagc |
| 987 | NM_014914 | CENTG2 | tgtttagatttccacgttacacaaacacac |
| 988 | NM_015050 | KIAA0082 | gtgtcttgcagaatcttggatcattaaaga |
| 989 | NM_015069 | ZNF423 | ccagttcaccggacagtgattaaaactatg |
| 990 | NM_015099 | CAMTA2 | gtttatacgaattgccattaaacatcgctg |
| 991 | NM_015147 | KIAA0582 | ttgttgtaaatcccaatctttactgccatg |
| 992 | NM_015149 | RGL1 | taatgcttttagctgctcgcatgcttgtct |
| 993 | NM_015158 | ANKRD15 | ggattcctttttaccgtgtcacatttactt |
| 994 | NM_015166 | MLC1 | tgggggcagaccagagagctcaagtttcag |
| 995 | NM_015196 | KIAA0922 | tatggactaccacagcgaataggaatgcaa |
| 996 | NM_015210 | KIAA0802 | ccaacaggagagatctagttttctcaaggt |
| 997 | NM_015261 | KIAA0056 | ctttattccatttgaaagtgtcaagcccat |
| 998 | NM_015335 | THRAP2 | gctggtacagtacactaaaagactttgata |
| 999 | NM_015436 | RCHY1 | tacgaattttttggtgcatagcatctcagc |
| 1000 | NM_015458 | MTMR9 | gtgtgtaatatttgcgtatggtgttcctct |
| 1001 | NM_015474 | SAMHD1 | ctaagcataagtgaaagacacctcccctca |
| 1002 | NM_015528 | RNF167 | ccttacccacacctatccttttgaggggct |
| 1003 | NM_015568 | PPP1R16B | tgaggtaacttccacgtagccccttgccac |
| 1004 | NM_015570 | AUTS2 | atcggcggagccttcttgtgtaatgtaaac |
| 1005 | NM_015636 | EIF2B4 | gtacctgttgttctacgagtcaagagcagt |
| 1006 | NM_015670 | SENP3 | tgagagaatacttgttgatttctgatgtgc |
| 1007 | NM_015710 | GLTSCR2 | agatgccggagactcgcccttcaataaaaa |
| 1008 | NM_015833 | ADARB1 | ttacaactgcatgagcttcctctcgcacaa |
| 1009 | NM_015855 | WIT-1 | gtctgaggttacaattagaactcctgaccc |
| 1010 | NM_015869 | PPARG | taaactacaattgtttggcaccctctcttc |
| 1011 | NM_015965 | GRIM19 | cgcgctgttgccactgttacaggcagaaac |
| 1012 | NM_016041 | DERL2 | acaccatcctccattcgacccattgacttc |
| 1013 | NM_016091 | EIF3S6IP | aactcagttaacaagttaaggaccgaagtg |
| 1014 | NM_016184 | CLECSF6 | ggtctgtacattgactgattcactttttca |
| 1015 | NM_016195 | MPHOSPH1 | ggatgtaataggtcaggtatttggtttact |
| 1016 | NM_016221 | DCTN4 | atgagctaagtgtcatgcatatttgtgaag |
| 1017 | NM_016308 | UMP-CMPK | gaaagaccccaatttaagccttgcttattt |
| 1018 | NM_016343 | CENPF | ctggtgatggattaacatatagcctttgtt |
| 1019 | NM_016570 | PTX1 | gtatcccccccatattgtgatgatgcaatc |
| 1020 | NM_016734 | PAX5 | agaacactcggaggggcgggaatcagaaga |
| 1021 | NM_017437 | CPSF2 | actatgtgtggaacaacccagtaatatcag |
| 1022 | NM_017448 | LDHC | tggagaacagaagatagcaggctgtgtatt |
| 1023 | NM_017617 | NOTCH1 | atgccagtgagggacgtcagacttggctca |
| 1024 | NM_017771 | PXK | cgttcacaacattctcttaagttctaacag |
| 1025 | NM_017787 | C10orf26 | cagacagaagcttgactgggtcttcaatga |
| 1026 | NM_017794 | KIAA1797 | ttagtacccgggccagttgagactgaaaca |
| 1027 | NM_018136 | ASPM | ttcaagacttaagccagattgggttttgag |
| 1028 | NM_018209 | ARFGAP1 | agggagtcccttttgatgggaagtgcagtc |
| 1029 | NM_018248 | FLJ10858 | atgcttttagtctcttgtaactggggagaa |
| 1030 | NM_018462 | C3orf10 | caatgacataacagagcacgcatcagaaga |
| 1031 | NM_018664 | SNFT | acatccaacaggcgccaaactcacagagcc |
| 1032 | NM_018951 | HOXA10 | gcgtggaaaaagacgatgtttatgttctta |
| 1033 | NM_019028 | ZDHHC13 | ttatgtcgatgccctgtagtttgaaagtga |
| 1034 | NM_019071 | ING3 | gaaggggaaagggcgttaaagtgatacatt |
| 1035 | NM_019102 | HOXA5 | gctgtactaacgaccaccaagacgccacca |
| 1036 | NM_019111 | HLA-DRA | ggcagaatataaacgacgcaccttcttccc |
| 1037 | NM_019841 | TRPV5 | agtaagctgtgaggctattctggctcccca |
| 1038 | NM_019846 | CCL28 | ctttcaggtcctgcatactgatgaaactac |
| 1039 | NM_019857 | CTPS2 | cccatgtcagtttctattgcattgagagcc |
| 1040 | NM_020310 | MNT | cgagacatgaatactatccatgtaataagc |
| 1041 | NM_020368 | SAS10 | cctaactataattattgggccagatacttg |
| 1042 | NM_020371 | AVEN | ccttgtttctagcctccttccttgcagtgt |
| 1043 | NM_020631 | KIAA0720 | aagcactgggaagtaatggggtgtggggtg |
| 1044 | NM_020657 | ZNF304 | aatagttgacagatctaactttcctagtgg |
| 1045 | NM_020841 | OSBPL8 | cagatagttataggccactgttttgtaatg |
| 1046 | NM_020843 | ZNF291 | gcttaataatgggcgataatgtcaagtgat |
| 1047 | NM_020892 | DTX2 | gcgaagacctgacgagagactgtaaaggaa |
| 1048 | NM_020944 | GBA2 | ccctgactgcgtgaaatgttcagggaggtc |
| 1049 | NM_020956 | PRX | aactagcactaaccctaagagggccgggag |
| 1050 | NM_020998 | MST1 | cttcttgtcagacataaagccatgtttcct |
| 1051 | NM_021019 | MYL6 | acagagcacaacaaaggacgagcagcgcca |
| 1052 | NM_021025 | TLX3 | gccgggcgcctgtattatactttgtacttt |
| 1053 | NM_021038 | MBNL1 | ctaccattaaggtgaaatcatggatcagat |
| 1054 | NM_021071 | DO | tgatcctatagctattgcatctctctcctt |
| 1055 | NM_021103 | TMSB10 | acttgtacgcctcccgtgagaccactctgt |
| 1056 | NM_021111 | RECK | agtgctgatgtagcatgcttgttgcaattg |
| 1057 | NM_021114 | SPINK2 | ttatcgcgcgtctatatggccccgatcacc |
| 1058 | NM_021603 | FXYD2 | cctctcttcgcttcacttggactgaccctg |
| 1059 | NM_021643 | TRIB2 | tagtgtgtggcatgtatgtgcagactcttg |
| 1060 | NM_021814 | ELOVL5 | cacatacaattacgtaccatatacagtgct |
| 1061 | NM_021906 | USP9X | agtgcaaagcaatgaaacgtccaatggtta |
| 1062 | NM_021949 | ATP2B3 | gaatggcttggtcaactgtgttcagttcat |
| 1063 | NM_021960 | MCL1 | tcaaatgtagtgtgtttccctaactttctg |
| 1064 | NM_022067 | C14orf133 | ctgacatgcctagactcctaataaaggtat |
| 1065 | NM_022127 | SLC28A3 | tgcacatgaatcatgagtcccttctccact |
| 1066 | NM_022161 | BIRC7 | tgcttggcgtgggggatggcttaactgtac |
| 1067 | NM_022334 | ITGB1BP1 | atctgttcataccgcatgacaccgtccact |
| 1068 | NM_022366 | TFB2M | ctggtttgcctttcagtttgttgaaatgta |
| 1069 | NM_022436 | ABCG5 | ggagttacctgatcacatcgagagagtgct |
| 1070 | NM_022438 | MAL | tgatggatcttaggttgcctgaaggcacgt |
| 1071 | NM_022469 | GREM2 | gctccttctggggttatcgtatgtacaaag |
| 1072 | NM_022549 | FEZ1 | taacaacagctcacagccaaatagagcaat |
| 1073 | NM_022716 | PRRX1 | tagtcatctatccgttcttcacttagcagg |
| 1074 | NM_024006 | VKORC1 | ggatttcagttccccgcgtcctcattaaat |
| 1075 | NM_024099 | MGC2477 | gggaagtaccttcattgggttgttctgaaa |
| 1076 | NM_024319 | C1orf35 | agccatgggcctatgagcggtctaactgtg |
| 1077 | NM_024408 | NOTCH2 | gcatattcctgactaaacgtagtaaggaaa |
| 1078 | NM_024424 | WT1 | aaggcaaagacacgtaaatctctgccaaca |
| 1079 | NM_024713 | C15orf29 | tgagttatttaagaggccagttttcaggac |
| 1080 | NM_024728 | C7orf10 | gctctggtattaatgaatctagtgcctttt |
| 1081 | NM_025263 | PRR3 | aatgggtcaggagttgtattggcaagaggg |
| 1082 | NM_030660 | ATXN3 | ttagtttcagtgttccctatccctcattct |
| 1083 | NM_030674 | SLC38A1 | gggtgatttaagtgagtcacaagtcacaaa |
| 1084 | NM_030915 | LBH | acttctatctgttcttttttgaggctcagg |
| 1085 | NM_030926 | ITM2C | gaggagggctgtaacgccttcagtcagtct |
| 1086 | NM_030935 | THG-1 | agacagcgacgatgatagtggctccggaag |
| 1087 | NM_031243 | HNRPA2B1 | tggtctttgtaagagtgtagaagcattcct |
| 1088 | NM_031423 | CDCA1 | gaaaagttgaagcgaatggaagtatcagaa |
| 1089 | NM_031966 | CCNB1 | gtaaaagtctaccaccgaatccctagtccc |
| 1090 | NM_032121 | DKFZp564K142 | tgagggctagacgtgtgctctgaagaatgg |
| 1091 | NM_033238 | PML | attccgtagggtcttgttcccacgaccgga |
| 1092 | NM_033512 | TSPYL5 | ggtaatttgacaccctgttaataacgcaat |
| 1093 | NM_033554 | HLA-DPA1 | atagcacatttccaactaataacaccccct |
| 1094 | NM_033642 | FGF13 | atacccaaatacgcaccgaataaactcttt |
| 1095 | NM_057168 | WNT16 | ggacatcgaagagaatttaacttagcagtt |
| 1096 | NM_058195 | CDKN2A | gttgtatttcgtcttatgtctcgagttcct |
| 1097 | NM_080284 | ABCA6 | tggccttatttttccaatcccagagcccac |
| 1098 | NM_080700 | TREX2 | atcgagcccatgtacttgccgcctgatgac |
| 1099 | NM_130439 | MXI1 | ggtggtgtagattgtatgagtaagaagtat |
| 1100 | NM_133259 | LRPPRC | caagctctgccctaggcttgttactttata |
| 1101 | NM_133378 | TTN | caacactttgttcgctcattttacgctgtc |
| 1102 | NM_138714 | NFAT5 | tgttctctgggggaatttcatttgcatcta |
| 1103 | NM_144578 | C14orf32 | agtttgagatactaggttttatcacctgct |
| 1104 | NM_144628 | TBC1D20 | aacttgtccatgtgccaagagggtggcgca |
| 1105 | NM_145698 | ACBD5 | gataaccatttctatcctgttccaaagtct |
| 1106 | NM_145867 | LTC4S | aggaacagcgggaagtactcgctgcagttc |
| 1107 | NM_147180 | PPP3R2 | ccttgaactgagagcctgtatctggattta |
| 1108 | NM_152739 | HOXA9 | accgccattgggctactgtagatttgtatc |
| 1109 | NM_152788 | EB-1 | tccttcactttagtggtctaatcaacagat |
| 1110 | NM_152827 | SNX3 | actacaccattggtttccaatactgcacac |
| 1111 | NM_152890 | COL24A1 | cacagcaaaccacacttaacctatctataa |
| 1112 | NM_156038 | CSF3R | ccaggcgatctgcatactttaaggaccaga |
| 1113 | NM_172020 | POM121 | gatttctagaactccctctttgcatagtgc |
| 1114 | NM_173156 | C1orf16 | catcctcactgtggagtaatgagggggagg |
| 1115 | NM_173216 | SIAT1 | gctgcctgtgtctatggggttctgtcttct |
| 1116 | NM_173609 | C15orf21 | tcaaccctcactaaactttgccggttcaga |
| 1117 | NM_173852 | KRTCAP2 | tccaccgagtctgtgtcaccacctgcttca |
| 1118 | NM_175739 | SERPINA9 | tgggatgttgctgggttaccatatttccat |
| 1119 | NM_175744 | RHOC | aggggggcatgaataaaggctacaggctcc |
| 1120 | NM_178586 | PPP2R5C | ctttttagtgtggctttagtatggcttcct |
| 1121 | NM_181311 | TAZ | agaaggcaggtgagcaaccagttggctagg |
| 1122 | NM_181336 | LEMD2 | gccccgtggtgaggccatgttctcataatc |
| 1123 | NM_181339 | IL24 | atatcacttccatgacataagtgctattgc |
| 1124 | NM_181430 | FOXK2 | agatcccaggtctctccgtggtatgtttgt |
| 1125 | NM_181493 | ITPA | gtgattcacttattcgccctccctactttg |
| 1126 | NM_181702 | GEM | atatagaattgggaactgatacatgtgtcc |
| 1127 | NM_182729 | TXNRD1 | gtggagtggaatgttctatccccacaagaa |
| 1128 | NM_182810 | ATF4 | tcaggagcgtcaatgtgcttgtacatagag |
| 1129 | NM_183395 | CIAS1 | gctttttgtagctggttgaaaattcaggaa |
| 1130 | NM_198232 | RNASE1 | ctcaaggcgggttaaacggttactcaaaag |
| 1131 | NM_198400 | NEDD4 | tgcgggacagtcagtaggatcattttcata |
| 1132 | NM_199335 | FYB | ataatacaggaacaggtatagatgtagcct |
| 1133 | NM_199423 | WWP2 | taactgaagtgtctgtacggaatgcccttt |
| 1134 | NM_201592 | GPM6A | gaaatgtagaacagagtgcttgcaaaatgt |
| 1135 | NM_201632 | TCF7 | gttttcaccatagcctacgttaacccattt |
| 1136 | NM_201998 | SF1 | aacactgcgctaaagcggggatgttccatt |
| 1137 | NM_212474 | FN1 | ctaatggagatagctttacactttctgctt |
| 1138 | R14777 | CYFIP2 | cataggatccaactggacaacgtgtgggat |
| 1139 | R56397 | PIK3C3 | tattacatggtacctgagttctgcttcctt |
| 1140 | R59027 | GRP58 | attttagtcccagccttgcagcttacacac |
| 1141 | R72151 | GNL3 | cattgtgagtggaagtagttatctggaata |
| 1142 | R79128 | MAP3K1 | ttgagtttgtttgcagttccctcagcttgc |
| 1143 | R86893 | C6orf110 | ctcccacgtctatgttcacatttgtggtcc |
| 1144 | T07281 | NRIP1 | aaagaatgtgctttgattcgaagggtctta |
| 1145 | T28925 | ITGAL | cgtgggacatctagtaggtgcttgacataa |
| 1146 | U60115 | FHL1 | catctaacgtttcagtgtatccttacagaa |
| 1147 | U79271 | AXT3 | atatgccacaacgcaccacgaccttcccag |
| 1148 | W04885 | MYL4 | ggcggtggggtctagattccacatttatgt |
| 1149 | X66087 | MYBL1 | atacttgcatggaaactgactacacataca |
| 1150 | AK024272 | | gaacattccaacagcagaatacatgttctt |
| 1151 | AK025231 | IGLC2 | gatgggatttcacctaattttaatgtggct |
| 1152 | AL080190 | | tctccttcgtgtgttcacctctcccccaga |
| 1153 | BU618233 | | agaggcaactttgcggtccatcacgcaatc |

TABLE 21
50mer polynucleotide probes, according to one embodiment

| | GenBank | | |
|---|---|---|---|
| | Accession No. | | |
| SEQ ID NO: | for Target gene | Symbol | 50mer |
| 1154 | AA594161 | MYH11 | atgaagttgagagcgtcacagggatgcttaacgaggccgaggggaaggcc |
| 1155 | AA766908 | MME | ttttagtccaaataacaagtaccaaagttttatcaagtttgggtctgtgc |
| 1156 | AB014540 | SWAP70 | ttaggagtcaaatagtctctgcatggtggggaggatcatgatggaatatg |
| 1157 | AF196185 | PARD3 | cacgtgcggcggtgtctaagtggtgttaccagtgtacgcgcagtgacctt |
| 1158 | AI597616 | MRPL33 | actgagggcggggatactgattcagaaaccctgtagcgtgtaataaaaga |
| 1159 | AI634809 | ARID5B | aagcttagctggtcatatttctacttgcttccttttagcttctgccttgc |
| 1160 | AI672553 | AKAP12 | gctggactgcattcacacatggcatgaaataagtcaggttctttacaaat |
| 1161 | AI809213 | RGS13 | atgagatttctatcgtacagggcaagcctacattgtggacatattctaga |
| 1162 | AK022231 | STAT1 | ataatatgcacacagtgctttccgtggcactgcatacaatctgaggcctc |
| 1163 | AK022293 | CTSD | gcacccctagctcttgggatgtttgtatccaactccgggcaatgtggggc |
| 1164 | AK055652 | C3orf6 | tgggttctgatgggaatatatacactcatctggactaggccaatggaagc |
| 1165 | AL080130 | FLJ14001 | atttagctgcataaaacgttcggctcatttatctgacatcttagtcacat |
| 1166 | AL833316 | MIR | tttttgggccttaggcgtttcgttcatcctgctaaggggatgaagcaaac |
| 1167 | AW291384 | STS-1 | caaatgcatcaattataccagggatgtatagtaagtcagggaactaatat |
| 1168 | BG993697 | SMYD3 | cctttccatgctacacaagcaagaaatgcctataaagacagacttgagtg |
| 1169 | BI769730 | HLA-DRB1 | gaaatatgcacaatcgcggtgtacgaggagcatacgccacacttatgatt |
| 1170 | BQ632574 | RASA1 | tcccgtgatttagcagcattgcatgagatttgcgtggctcattcagatga |
| 1171 | BX504817 | SFRS7 | catcaagtttattcggtagtttgaccgctagtatgttggaagttatttgg |
| 1172 | H53164 | IRF8 | cagcaccaatcccgcattcagaacctcccagtgaaagggcagccttcatt |
| 1173 | H57732 | TGFBR2 | aatgtgtccaaaaggttggatggcaaggtttgggtgctactgccttggca |
| 1174 | L29376 | 3.8-1 | tatatgtacagtttcacatatgtataaaaacagtagtttgggggcctctt |
| 1175 | M80899 | AHNAK | aaaggccctaaatttaagatgccagacatgcacttcaaggcccccaagat |
| 1176 | NM_000038 | APC | aagcagcctagcacagactaagcattgagcataataggcccacataattt |
| 1177 | NM_000043 | FAS | ttcgtgagctcgtctctgatctcgcgcaagagtgacacacaggtgttcaa |
| 1178 | NM_000061 | BTK | atttccagtccggtggtccccaccggaagtcctgatgtatagcaagttca |
| 1179 | NM_000075 | CDK4 | cagaggtagaacgcatatcgacgtagacttatcgttacaactaattagaa |
| 1180 | NM_000109 | DMD | tcagcctatgctgctccgagtggttggcagtcaaacttcggactccatgg |
| 1181 | NM_000189 | HK2 | atacagtggatctcaatcttcggggtgtgatgaatagcgaatcatctcaa |
| 1182 | NM_000237 | LPL | tcgtgcctataaatagtaggaccaatgttgtgattaacatcatcaggctt |
| 1183 | NM_000269 | NME1 | gtggaaatgcgtgtcacggccccgtcttaccgctagttccctgcatagca |
| 1184 | NM_000271 | NPC1 | ggaatgcacagttgacttgggaagcagtattactagatctggaggcaacc |
| 1185 | NM_000295 | SERPINA1 | tgctccctccgtctctaccaggaatggccttgtcctatggaaggcactgc |
| 1186 | NM_000313 | PROS1 | taatatatcggatacaggccctaagtctatgttccgatcaacaatctcat |
| 1187 | NM_000397 | CYBB | cccttggagccacaaatgtttagaactcttcaacttcggtaatgaggaag |
| 1188 | NM_000405 | GM2A | gtataactgtgagtcttggagccatagtaatgtttctcatgccccccaaa |
| 1189 | NM_000424 | KRT5 | ttatccttttctggagagtagtctagaccaagccaattgcagaaccacat |
| 1190 | NM_000483 | APOC2 | catacctgcccgctgtagatgagaaactcagggacttgtacagcaaaagc |
| 1191 | NM_000576 | IL1B | aagaatctgtacctgtcctgcgtgttgaaagatgataagcccactctaca |
| 1192 | NM_000579 | CCR5 | ccttttgctcttaagttgtggagagtgcaacagtagcataggaccctacc |
| 1193 | NM_000584 | IL8 | ctattaaaacagccaaaactccacagtcaatattagtaatttcttgctgg |
| 1194 | NM_000585 | IL15 | ttatttcacttgagtccggagatgcaagtattcatgatacagtagaaaat |
| 1195 | NM_000600 | IL6 | gagcccagctatgaactccttctccacaagcgccttcggtccagttgcct |
| 1196 | NM_000626 | CD79B | tagtgacgctgcggacaggggaagtgaagtggtctgtaggtgagcaccca |
| 1197 | NM_000627 | LTBP1 | gctgctgtacatcaggcgcgggatggggagataactgcgaaatcttcccc |
| 1198 | NM_000633 | BCL2 | atggcaaatgaccagcagattcaaatctatggtggtttgacctttagaga |
| 1199 | NM_000655 | SELL | gtagcctcgccgtctgtgaattggaccatcctatttaactggcttcagcc |
| 1200 | NM_000713 | BLVRB | aatgacctcagtcccacgacagtgatgtccgagggcgcccggaacattgt |
| 1201 | NM_000785 | CYP27B1 | cttgcccctaggaaggtgaatctgccctagcctggtttacggtttcttat |
| 1202 | NM_000788 | DCK | cttaagtataaaccttatgaactacagtggagctacactcattgaaatgt |
| 1203 | NM_000853 | GSTT1 | aatctcaggatggacgcacctccgatgaattcctctgacattctgccagg |
| 1204 | NM_000877 | IL1R1 | gaaatagccaccgtctacagatggcttagttaagtcatccacagcccaag |
| 1205 | NM_000917 | P4HA1 | tcagcgtcacataaaaagagcatgtaggatgggacatattgggatgtatt |
| 1206 | NM_000927 | ABCB1 | atacatggttttccgatccatgctcagacaggatgtgagttggtttgatg |
| 1207 | NM_001067 | TOP2A | gttatattgataaccatgctcagcaatgagctattagattcattttggga |
| 1208 | NM_001068 | TOP2B | tgtccttaagcacaacagtcataccagcacaacgcaatgcttatgtcct |
| 1209 | NM_001110 | ADAM10 | ggaagacatttcaacctacgaatgaagagggacacttcccttttcagtga |
| 1210 | NM_001154 | ANXA5 | tatagtcccagatgtatctcccttaatcatggaataaagagaggtggcaa |
| 1211 | NM_001166 | BLRC2 | agagtataatggaagatagcacgatcttgtcagattggacaaacagcaac |
| 1212 | NM_001196 | BID | tgctttcctcaaacaggatactgcctgctgtaagaccatcctctatggtt |
| 1213 | NM_001230 | CASP10 | ttattgcctctttcacattgaaacccaggagtggataacactggcttcag |
| 1214 | NM_001238 | CCNE1 | tatggtatacttgctgcttcggccttgtatcatttctcgtcatctgaatt |
| 1215 | NM_001242 | TNFRSF7 | acctgaattcccaccgcagagagtgttgtggccgtgaggacttttctgtc |
| 1216 | NM_001254 | CDC6 | ttttagactcgttgagtttcttgggcactcccaagggcgttggggtcata |
| 1217 | NM_001350 | DAXX | gggataacatttggaggaaggtgggaagcagatgactgaggaagggatgg |
| 1218 | NM_001425 | EMP3 | cagctttgcaccagcgtggcggtgtttactggcggccttgatctatgcca |
| 1219 | NM_001552 | IGFBP4 | ggcttgactggatggaaggagacttaggaacctaccagttggccatgatg |
| 1220 | NM_001558 | IL10RA | actgacttgtctaattcgtagggatgtgaggttctgctgaggaaatgggt |
| 1221 | NM_001618 | PARP1 | ggtgtagacgttcctcttgggaccgggatttcatctggtgtgatagacac |
| 1222 | NM_001621 | AHR | catgaggtatctaaggatttagaccagaggtctagattaatactctattt |
| 1223 | NM_001647 | APOD | tctgcccctagtgtgtgttgcgtgtccgctccacggcgtacatgttgctt |
| 1224 | NM_001663 | ARF6 | tggttagctggttaggaccagtaactggattgcgaccactatgataatat |
| 1225 | NM_001706 | BCL6 | tgtttgtctcatcccattctgcgtcatgcttgtgttataactactccgga |
| 1226 | NM_001760 | CCND3 | cctgttgacacaggtctttcctaaggctgcaaggtttaggctggtggccc |
| 1227 | NM_001762 | CCT6A | agatgatggaaggtgtggtgactaagggccacggttattgggtgaaattt |
| 1228 | NM_001770 | CD19 | acacctgactctgaaatctgaagacctcgagcagatgatgccaacctctg |
| 1229 | NM_001771 | CD22 | ttggaagtgaggcattgcacggggagacgtacgtatcagcggccccttga |
| 1230 | NM_001779 | CD58 | gttcctttcatcacaacaaacgttatggagattatgcgataccaggcact |
| 1231 | NM_001781 | CD69 | gatgtggcaaatctctattaggaaatattctgtaatcttcagacctagaa |
| 1232 | NM_001782 | CD72 | agttgactgatgatacacaacgcactaggacttatgctcaaagctcaaaa |
| 1233 | NM_001826 | CKS1B | gatcatgtcgcacaaacaaatttactattcggacaaatacgacgacgagg |
| 1234 | NM_001827 | CKS2 | cagctaaacactcctgcggtcgtatggtcgacaacaatcacacataaaat |
| 1235 | NM_001831 | CLU | acttaaagcttagcttatgtccaaggtaagtattttagcatggctgtcaa |
| 1236 | NM_001852 | COL9A2 | ctgaaggaatagggcggctttccttccagcgagcatcattcggctgttac |
| 1237 | NM_001853 | COL9A3 | accctcatcgggctgtcgcctgacagcatacctcaaaaggccctagctaa |
| 1238 | NM_001877 | CR2 | cttcttaccttcttgattgtcattaccttatacgtgatatcaaaacacag |
| 1239 | NM_001888 | CRYM | tttatgtttgtagttggaaagcaaagctaggtagccatttcttctgttct |
| 1240 | NM_001894 | CSNK1E | gccagcccgaattcccgttctcctgtgtctactaacaaggacatgggggt |
| 1241 | NM_001951 | E2F5 | gacattccactttcctaggttataggaaagatctgtttatgtagtttgtt |
| 1242 | NM_001967 | EIF4A2 | aatatccttgctgcgtaaactgcacgggtctttaagctccagacgttttc |
| 1243 | NM_002002 | FCER2 | tgtgtgcaacacgtgccctgaaaagtggatcaatttccaacggaagtgct |
| 1244 | NM_002051 | GATA3 | tgaataagccattctgactcatatcccctatttaacagggtctctagtgc |
| 1245 | NM_002095 | GTF2E2 | acagaaaaagcgacgctttaagactcataacgaacacttggctggagtgc |
| 1246 | NM_002101 | GYPC | taactgagcatacccagtgggccaacaagcacgctctaaatacaaaacca |
| 1247 | NM_002105 | H2AFX | gcctacccaccaatccttaacgcgcctcaccggaccttaacattccaccc |
| 1248 | NM_002120 | HLA-DOB | cattgccaagggcattgtccagaaactcccctgagaccttactccttcca |
| 1249 | NM_002122 | HLA-DQA1 | gccacccggctacctaattcctcagtaacctccatctaaaatctccaagg |
| 1250 | NM_002155 | HSPA6 | tatcttatatatctgtattcgcatggtaacatatcttcggccttcctgag |
| 1251 | NM_002189 | IL15RA | cggtacatttgtaactctggtttcaagcgtaaagccggcacgtccagcct |
| 1252 | NM_002211 | ITGB1 | gttatactgtggctatgcaacagctctcacctacgcgagtcttactttga |
| 1253 | NM_002217 | ITIH3 | tgactacattgtccccaacctgttttgagtagacacaccagctcctgttg |
| 1254 | NM_002250 | KCNN4 | taactcaccagcctctgctcttatctttgtaataaatgttaaagccagaa |
| 1255 | NM_002305 | LGALS1 | tggcggcggatggagacttcaagattaagtgcgtggcctttgagtgaagc |
| 1256 | NM_002350 | LYN | tgatttcatgtgcggggatcatctgccgtgcctggatcctgaaatagagg |
| 1257 | NM_002358 | MAD2L1 | ccagaacatgttacaccactcctacgatggcttgcccgattcgatgcccc |
| 1258 | NM_002426 | MMP12 | aaatagttaccttcaaaggccaagagaattctatttgaagcatgctctgt |
| 1259 | NM_002467 | MYC | tcctcaagaggtgccacgtctccacacatcagcacaactacgcagcgcct |
| 1260 | NM_002526 | NT5E | gcagcaaaataatagcctcggttctatgcatatatggattagctataaaa |
| 1261 | NM_002567 | PBP | accccccacgatccaagagaaaaaccgatagcacgtaacccgctgctcag |
| 1262 | NM_002646 | PIK3C2B | gctcattttattatgacctatatgctcctgatttaaagagatctgtgtac |
| 1263 | NM_002661 | PLCG2 | agaagatatgttcagcgatcccaactttcttgctcatgccacttacccca |
| 1264 | NM_002692 | POLE2 | ttccacccactgagccctctagtactactagggcatactatggaaatatt |
| 1265 | NM_002738 | PRKCB1 | aaattgtgagtaagctttgcagttactgtgaactattgtctcttggagga |
| 1266 | NM_002752 | MAPK9 | acgaagctctgcgtcacccatacatcactgtttggtatgaccccgccgaa |
| 1267 | NM_002832 | PTPN7 | ctcaaataataccctgggtatgcaggacccactataccttgcatttgctg |
| 1268 | NM_002838 | PTPRC | cttatgattataaccgtgttgaactctctgagataaacggagatgcaggg |
| 1269 | NM_002891 | RASGRF1 | cccggggacatgtgctagatgatactgtacatattcgtttggtttcactg |
| 1270 | NM_002892 | ARID4A | agaggcagaagtcaaaacggggacgacctcctttaaaatcaaccctctca |
| 1271 | NM_002943 | RORA | acctaataaacacaacttttactaattatctatactactacaa |
| 1272 | NM_002966 | S100A10 | acggatcgctcccatgtttggtccacttaccacccactttgtgtttcctt |
| 1273 | NM_002967 | SAFB | cttggccatcaaataagtaggcatagagtgtgactcgccctctcttggtc |
| 1274 | NM_003037 | SLAMF1 | aggccctccacgttatctaggagatcgctacaagttttatctggagaatc |
| 1275 | NM_003088 | FSCN1 | actttgacatcgagtggcgtgaccggcgcatcacactgagggcgtccaat |
| 1276 | NM_003177 | SYK | ctgcaagaaatgtgttgctagttacccaacattacgccaagatcagtgat |
| 1277 | NM_003217 | TEGT | acttccttcctgttaaacccctgttaactctccataaatttggtgattct |
| 1278 | NM_003226 | TFF3 | tgtatccgaaacacggtgacaccgctggtgccggctaaacccccattgtg |
| 1279 | NM_003243 | TGFBR3 | caggtgagaacatccataatttggggccctgagttttacccagactcaag |
| 1280 | NM_003254 | TIMP1 | atacatcttggtcatcttgatctcataacgctggtataaggtggtctggt |
| 1281 | NM_003290 | TPM4 | ccttatgagaagaggccagaacttggttatcctcttccaatgtgaggaca |
| 1282 | NM_003299 | TRA1 | ggatctaaaaagagcgattacattaagctctatgtgcgccgtgtattcat |
| 1283 | NM_003332 | TYROBP | tgagagaccagaccgctccccaatactctcctaaaataaacatgaagcac |
| 1284 | NM_003362 | UNG | gttcactgatgcagttgtgtcctggctaaatcagaactcgaatggccttg |
| 1285 | NM_003451 | ZNF177 | gactgatgtactgtggcagacagaactagttgctgtctcaatatccattc |
| 1286 | NM_003648 | DGKD | tcttgccaagcgcagtcgcagtggtaaattccgcctcgtgaccaagttta |
| 1287 | NM_003656 | CAMK1 | caaagagtttggcatcattctcgtcatagaagggagggtaaccgcagagc |
| 1288 | NM_003810 | TNFSF10 | aaaagagagaagaggcaccactaaaagatcgcagtttgcctggtgcagtg |
| 1289 | NM_003875 | GMPS | tctggctaggcttatacctcgcatgtgtcacaacgttaacagagttgttt |
| 1290 | NM_003959 | HIP1R | acatccgggagattggagacctgtggggacatttgcatgaccgctacgga |
| 1291 | NM_004049 | BCL2A1 | aatctggctggatgacttttctagaagttacaggaaagatctgtgaaatg |
| 1292 | NM_004073 | PLK3 | ataagcctgagccttagctcccagctagggggcgttatttatggaccact |
| 1293 | NM_004091 | E2F2 | acccaaatactcacaagggtcttatcagacgcccgtcttaaagtccagca |
| 1294 | NM_004117 | FKBP5 | ttcagtccttacgggtaaacagattgagcatggctctctattccctcagc |
| 1295 | NM_004126 | GNG11 | atcacccaagagccataatacacctattataagcgaccagcataataaag |
| 1296 | NM_004184 | WARS | ggccctcacagatgtctaggcaggcctcatttcatcacgcagcatgtgca |
| 1297 | NM_004271 | LY86 | tcagcccgcgcgtggaaccgagacaagtaaacaagacgccagatctctga |
| 1298 | NM_004289 | NFE2L3 | cactgaccatgaatctagttcccatcatgacttagaaggtgctgtaggtg |
| 1299 | NM_004322 | BAD | cctagcacaaaataagggcataagaaatactagttacatcacaaggtcag |
| 1300 | NM_004354 | CCNG2 | tcacacatgcaaattgtcagcttattgagacaacccacttagattcatat |
| 1301 | NM_004356 | CD81 | cgtcgccttcaactgtaatcacaacatcctgactccgtcatttaataaag |
| 1302 | NM_004358 | CDC25B | gagattactctaaggccttcctcctacagacagtagacggaaagcaccaa |
| 1303 | NM_004422 | DVL2 | aggaagctgggagatttgggccatgtagctgcctttgttactctatttat |
| 1304 | NM_004445 | EPHB6 | agcccaggactcggggtgaagtattacatcgacccctccacctacgagga |
| 1305 | NM_004510 | SP110 | tgatggttttagcaatacctaaatccgtgatatcatcagaggttgcaaaa |
| 1306 | NM_004529 | MLLT3 | gaaatcaacgttaccgccatttgatgatattgtggatcccaatgattcag |
| 1307 | NM_004556 | NFKBIE | cagacccttgtccttgctgtggaaatgatgagggatggagggacaagagg |
| 1308 | NM_004577 | PSPH | tgtgatcaggcaacaagtcaaggataacgccaaatggtatatcactgatt |
| 1309 | NM_004619 | TRAF5 | attctgggccggtaccaggttggtattactcatgaacgatatctgctttt |
| 1310 | NM_004635 | MAPKAPK3 | ttgatcctctacaactggtgttgctcccgttatgcggacagaagacagcc |
| 1311 | NM_004737 | LARGE | gttccaacaagcaataccgcatctgtctcaaaaccctcaaggaagagttt |
| 1312 | NM_004844 | SH3BP5 | gctatcattctagggctggatttccctaactccagttattctgctgctgc |
| 1313 | NM_004851 | NAPSA | gcgcagcgggtggtcgcggaggtcctgctacccagtaaaaatccactatt |
| 1314 | NM_004951 | EBI2 | gaggatgctgaaacggcaagtcagtgtatcgatttctagtgctgtgaagt |
| 1315 | NM_005013 | NUCB2 | gcggcaacaagtgatctggaacactatgacaagactcgtcatgaagaatt |
| 1316 | NM_005077 | TLE1 | atgggttgtaggattgaggaacggaatctgccgactcacatgacagccca |
| 1317 | NM_005127 | CLECSF2 | tttgtacctgtctggttaattctgcttacgtgtcaggctacacataaaag |
| 1318 | NM_005211 | CSF1R | gctgactcatcctaactaacagtcacgccgtgggatgtctctgtccacat |
| 1319 | NM_005213 | CSTA | aacactttgggtacatgctgctaaaagcccgtcagctcgtcatccttgtt |
| 1320 | NM_005214 | CTLA4 | caccttatatttacgtatgagacgtttatagccgaaatgatcttttcaag |
| 1321 | NM_005271 | GLUD1 | aacagtagtgtccccatgaagtgctagataatatatgtgtaagagtcagc |
| 1322 | NM_005292 | GPR18 | gtcattagtgtcatgctataccgtaattaccttcgaagcatgcgcagaaa |
| 1323 | NM_005347 | HSPA5 | aaggctctcactaccgtggaccacctagtctgtaactctttctgaggagc |
| 1324 | NM_005348 | HSPCA | agttgacaattctgcatgtactagtcctctagaaataggttaaactgaag |
| 1325 | NM_005415 | SLC20A1 | ctccaagaagcgaattcgaatggacagttacaccagttactgcaatgctg |
| 1326 | NM_005449 | TOSO | tagttgacaggtaagctgtaggcatgtagagcaattgtcccaatgccact |
| 1327 | NM_005461 | MAFB | tttaactttagcatgttgttgatcatggatcatactccccttgtttcttt |
| 1328 | NM_005531 | IFI16 | ctgctttgaattggcaccgaaaagtgggaataccggggagttgagatctg |
| 1329 | NM_005582 | LY64 | ccaaacgggaatatccaatctcgagtttattccagtgcacaatctggaaa |
| 1330 | NM_005601 | NKG7 | cgtccactcccccgctatccaacgtcacatctaagcactatgacatgcac |
| 1331 | NM_005606 | LGMN | ctggcactcccccacgtacgagtatgcgttgagacatttgtacgtgctgg |
| 1332 | NM_005610 | RBBP4 | gcatctctagcccactttatatcctgccaccagtacttcttgtaatccca |
| 1333 | NM_005623 | CCL8 | tgttgtggggtcctcccatggatcatcaaggtgaaacactttggtattct |
| 1334 | NM_005638 | SYBL1 | gcatgttttagggatcaattacctaactgttccttggtctatttatgtat |
| 1335 | NM_005652 | TERF2 | acaggagcatggttcctaataatagcccctgatagtctgctctttctttc |
| 1336 | NM_005658 | TRAF1 | atccctactcaccgagtgttgagcccaaggggggatttgtagaacaagcc |
| 1337 | NM_005856 | RAMP3 | tctcatcccgctgatcgttatacccgtcgttctgactgtcgccatggctg |
| 1338 | NM_005935 | MLLT2 | ttgttttgggtcccacttaggattaacggatgtaaggtattttcctgtgc |
| 1339 | NM_005950 | MT1G | acattatcaactagatactacacataccagcaccactactcacactacag |
| 1340 | NM_005951 | MT1H | gtaaaatccaggattttatattcctacaactccgactcatttgctacatt |
| 1341 | NM_006006 | ZBTB16 | gcctgagaagcatctgggcatctactccgtgttgcccaaccacaaggctg |
| 1342 | NM_006014 | DXS9879E | acaacaccagcgcccaatatacgcgaatgaggcgccctatacccgcctct |
| 1343 | NM_006115 | PRAME | tctaagtggggtcatgctgaccgatgtaagtcccgagcccctccaagctc |
| 1344 | NM_006162 | NFATC1 | tgcttattacgtatgattactcacagcgatctattgttccatataaccaa |
| 1345 | NM_006164 | NFE2L2 | ggccagtaaacacctgttcctgagtgatacatctttaaggagccggtaga |
| 1346 | NM_006195 | PBX3 | ttctgtagcttagagtgctcacttactacctctgaacaatactcacgctg |
| 1347 | NM_006218 | PIK3CA | actgtgtgggacttattgaggtggtgcgaaattctcacactattatgcaa |
| 1348 | NM_006254 | PRKCD | tccgtgtggacacgccacattatccccgctggatcaccaaggagtccaag |
| 1349 | NM_006257 | PRKCQ | gacaagtaatcactaacccgttttattctattcctatctgtggatgtgta |
| 1350 | NM_006274 | CCL19 | acctcagccaagatgaagcgccgcagcagttaacctatgaccgtgcagag |
| 1351 | NM_006317 | BASP1 | ggccccggcagctaattccgaccaaaccgtaaccgtgaaagagtgacaag |
| 1352 | NM_006332 | IFI30 | atgcaccgcttgtcaatgtgaccctctactatgaagcactgtgcggtggc |
| 1353 | NM_006475 | POSTN | aagactgcttcagggagacacacccgtgaggaagttgcaagccaacaaaa |
| 1354 | NM_006498 | LGALS2 | ctgtgctcagggtctcaactcgtggtcaagcttatatcctagaatattac |
| 1355 | NM_006558 | KHDRBS3 | cagccaccgaatgcttaagaactcatctaagccagtcctctcattgattg |
| 1356 | NM_006620 | HBSIL | ttagatttaagtctgactgaggcatgttcaaagacactacactgcctctc |
| 1357 | NM_006644 | HSPH1 | gctcaggctaaaaagagtcttgatcaggatccagttgtacgtgctcagga |
| 1358 | NM_006734 | HIVEP2 | gaactaaggctacacagagggtcgcacttggactctgagggttgggtgtg |
| 1359 | NM_006763 | BTG2 | tacaatccaaatttgtcgtagacttgtgcaatatatactgttgtgggttg |
| 1360 | NM_006841 | SLC38A3 | tgcggcatgtgcttattgccgttggcctgctcacttgtatcaacctgctg |
| 1361 | NM_006889 | CD86 | ataaatttgacctgctcatctatacacggttacccagaacctaagaagat |
| 1362 | NM_006892 | DNMT3B | agggctggagtctgcacgggacctattagagtattttccacaatgatgat |
| 1363 | NM_006986 | MAGED1 | tccttctaaacacagctatctagagagccacatcctgttgactgaaagtg |
| 1364 | NM_007311 | BZRP | cctctactgactcctccggtgagggaagctattaaagcagaaggggttgc |
| 1365 | NM_007360 | KLRK1 | cagagaagagattacaccagcggtaacactgccaaccgagactaaaggaa |
| 1366 | NM_007361 | NID2 | ctgatgagtatctcccagaacaacgatctcacctctacgggataactgca |
| 1367 | NM_012203 | GRHPR | gtattactcattagtgcacaagtattacttagtgcacaatatacagtctc |
| 1368 | NM_012448 | STAT5B | agtttcagctccgtgtgtgagatgtgcaggttttttctagggggtagggg |
| 1369 | NM_012452 | TNFRSF13B | actgacacctgtctgggggcgttctacgctggtgattgcccttagctcct |
| 1370 | NM_013230 | CD24 | actccccgaagtcttttgttcgcatggtcacacactgatgcttagatgtt |
| 1371 | NM_014246 | CELSR1 | agtggaacgtgccggaactttttgtccattccctagtaggcctgccacag |
| 1372 | NM_014257 | CD209L | cacggggtaactgcgatcattgcacttggtgttcacagtgggccaggcac |
| 1373 | NM_014338 | PISD | acctgcatcactaccgcaacctcagcgagttcttccggcgcaagctgaag |
| 1374 | NM_014456 | PDCD4 | gaattagcactggatactcctagagcaccacagttggtgggccagtttat |
| 1375 | NM_014686 | KIAA0335 | gggccagtcacatcagggctctccgttggtgacaaggcataattctgctg |
| 1376 | NM_014762 | DHCR24 | attactacaagccgtggttctttaagcatgtggagaactatctgaagaca |
| 1377 | NM_015187 | KIAA0746 | gaggatcctgcgttaatctatgactatgccattgtgctattcaagggtca |
| 1378 | NM_015641 | TES | ccctaataccaaggcgctacgtttattgcctcgtcttattcactgacctt |
| 1379 | NM_015866 | PRDM2 | aaaatgccttccgacgaatgggacagcccaaaaggcttaactttagtgtt |
| 1380 | NM_016076 | PNAS-4 | cttcacttttgctaccttgatatagcattgggctatcatgttacaacatt |
| 1381 | NM_016081 | KIAA0992 | accgcacttatatgcattgctagtatggaatttaagataccatacacagt |
| 1382 | NM_016187 | BIN2 | acacttgcctatacagagacataaccacacacactcagaggatagtgaac |
| 1383 | NM_016336 | UBE2J1 | tacccaacctgtagctaagaatacctccatgagccctcgacagcgccggg |
| 1384 | NM_016628 | WAC | gcaggcatcaagattacgcgaagaagcgcataacatgggaactattcaca |
| 1385 | NM_017530 | LOC55565 | cgccacctcttaatccactcggaggaccagcgatctaactgtgctgtgtg |
| 1386 | NM_017784 | OSBPL10 | caagttcatgggcatgtccgtgggggtctctatgataggggaaggtgtgt |
| 1387 | NM_017935 | BANK1 | ctcacattcagagttccagtcattattgttacatcatgtttgcagaaacc |
| 1388 | NM_017955 | CDCA4 | atgtcattcataaccccttgatctgtagtgtcatgggtgctgccgcaggc |
| 1389 | NM_018442 | IQWD1 | ctggtcaccattagaagagtcaaggatttttaaccgaaaacttgctgatg |
| 1390 | NM_018842 | BAIAP2L1 | ctgtgaaactccgcccgactgtgacgaatgatcgctcggcacccatcatt |
| 1391 | NM_020151 | STARD7 | acaagtgcgaaggagttgcggttgctccatgttctgacttagggcaattt |
| 1392 | NM_020199 | C5orf15 | ctcctgcttgtgtccgccgctctatccagtgttgtatcacggactgattc |
| 1393 | NM_020529 | NFKBIA | catgccgataagtaaacgtcgaacaattgcccttcaaccactccctctaa |
| 1394 | NM_020992 | PDLIM1 | tggacatcagccaaatttgaacccaatcaaatataacgtgtctgacactg |
| 1395 | NM_021813 | BACH2 | tgtgtgtgcaaatcctgggatgcccgtttcacgctctgacataaagacat |
| 1396 | NM_021822 | APOBEC3G | ctactaatccagcgacaatttgaatcggttttgtaggtagaggaataaaa |
| 1397 | NM_021950 | MS4A1 | accacaccccatgagggaagctctaaatagccaacacccatctgtttttt |
| 1398 | NM_021966 | TCL1A | gtttctgttcaccttcgtttactatgcctgtgtcttctccaccacgctgg |
| 1399 | NM_022552 | DNMT3A | atgtacgtcggggacgtccgcagcgtcacacagaagcatatccaggagtg |
| 1400 | NM_023037 | 13CDNA73 | aaaaggcaatcgggcactggaccagtttaccctggcgagctttggagaag |
| 1401 | NM_024708 | ASB7 | aacacacggaactatgaaggacagaccccattggctgtttcaataagtat |
| 1402 | NM_025113 | C13orf18 | gaattccttctcaccagaggtatttgtgctgcctgttgatgtagaaaagg |
| 1403 | NM_025216 | WNT10A | cgagacacaacacttgtcctcttggaggttacattcttgctggggggagg |
| 1404 | NM_030666 | SERPINB1 | ttttctttattcggcataattcctcaggtagcatcctattcttggggaga |
| 1405 | NM_030764 | SPAP1 | tgatgccggcaaatattactgtagagctgacaacggccatgtgcctatcc |
| 1406 | NM_030775 | WNT5B | gctgtgtataagatggcagacgtagcctgcaaatgccacggcgtctcggg |
| 1407 | NM_031305 | ARHGAP24 | gttacaaaaggcataatcggaaatagagactacatacttgagtttatggg |
| 1408 | NM_031942 | CDCA7 | caatgtttgatgcataattggaccttgaatcgataagtgtaaatacagct |
| 1409 | NM_032991 | CASP3 | tttcttgttgctcaaaaaatgagctcgcatttgtcaatgacagtttcttt |
| 1410 | NM_033208 | TIGD7 | aacagaaacgctcagccggtgttccggtaagaggcgtggagcttcaggct |
| 1411 | NM_033274 | ADAM19 | acccccaacgcgtcctctcacaccgtccactgtgcgtttgtatgtgtctg |
| 1412 | NM_057735 | CCNE2 | catgtaggttgcttggtaataacctttttgtatatcacaatttgggtgaa |
| 1413 | NM_078467 | CDKN1A | ctgaagtgcttagtgtacttggagtattggggtctgaccccaaacacctt |
| 1414 | NM_079421 | CDKN2D | gcttgttttccagtctcttgtacagcgttttaaaagaaatggattctatt |
| 1415 | NM_080593 | HIST1H2BK | ggggcgattctcttgttgttccgatagtgtctcgcgggcgaaaccaccag |
| 1416 | NM_138340 | ABHD3 | ggaattccagtattgtgtctaaattctgtggatgatgttttctcacccag |
| 1417 | NM_138379 | TIMD4 | acgaagacggcctttttaccctctaacaacgcagtagcatgttagattga |
| 1418 | NM_139207 | NAP1L1 | aggtagaagtagaggcctaccttgaattaagggttatactcagtttttaa |
| 1419 | NM_139276 | STAT3 | gtgatatccagtggcacttgtaatggcgtcttcattcagttcatgcaggg |
| 1420 | NM_144646 | IGJ | gaataagcctaacacagtagaaattgctgtatggtgtagataaaacaatc |
| 1421 | NM_145804 | ABTB2 | ttgttcaatcactgatgggcaaaggggagatgtagctaactggtggccct |
| 1422 | NM_152785 | GCET2 | ctgatggccttgtacagagaacccgatgattgatgagggtatagcacagc |
| 1423 | NM_172373 | ELF1 | taaaccaccacgaccagattccccagccactacgccaaatatatctgtga |
| 1424 | NM_173624 | FLJ40504 | aaaaaattggccgtcaaaacctgagtcctgtcctcttgccctcctccccg |
| 1425 | NM_175870 | LOC90925 | ctgtcataaacattggatttcccgcggagtaacctggcgacatatttaga |
| 1426 | NM_177968 | PPM1B | atctgtattgaactttcggccctagaaaccagtggagttatttcaccaca |
| 1427 | NM_181443 | BTBD3 | gcaaattggtagaccttataagaaggcacttgtttgtaagccagagaaga |
| 1428 | NM_181838 | UBE2D2 | aaaacacagaggcattgctatttggtaagttaagcttctgtgattgtaat |
| 1429 | NM_182746 | MCM4 | tacattgcctacgcgcacagcaccatcatgccgcggctaagtgaggaagc |
| 1430 | NM_182776 | MCM7 | atatttgccaccgtccgtgaactggtctcagggggccgaagtgtccggtt |
| 1431 | NM_198833 | SERPINB8 | catctatgctttgtagtatgactcctgtcactcagtacaattattttgag |
| 1432 | NM_201593 | CACNB2 | acagttcacactgattgtgacacattcttagtagctagtgtctgttctag |
| 1433 | R99527 | MGC39372 | gggtcaaaccatacatcgcttcgtctctgtagtgatgacttcctgtcctc |
| 1434 | T66903 | LOC54103 | gtccagtgtctccctttgcataacctgctgcattgcattgacagtggagt |
| 1435 | U37028 | ITGAD | gctgagcatcgataccgtgtgaataacctcagccagcgagatctggccat |
| 1436 | W56129 | SFRS9 | tactatggagatcaacagttactgtgactgagtcggcccattctgtttag |
| 1437 | X58529 | IGHM | gagagggtttattacgattttgggagtggtcgattatgtactactttgac |
| 1438 | AA188785 | LOC286025 | gggaaggaggactccggtcttaactcccacgctaactttagctcaagtgg |
| 1439 | AA347638 | BCR | ttccatggagacgcagatggctcgttcggaacaccacctggatacggctg |
| 1440 | AA601122 | SECTM1 | gcgtttgtgtctcgggttaaaataaggttccgtccgcgtgctgggtcaga |
| 1441 | AA642467 | SLC2A14 | tggtcttggctttggtccacttgctgaacattttggcttcaggaagcagt |
| 1442 | AA682722 | LARP | agaatacctcggcaaattccgacgtcttgaagacttccgagtagatcccc |
| 1443 | AA757235 | LOC399933 | cttttggagtagtgtattctatgggtccagtccctttcatgcagccatta |
| 1444 | AA765313 | FZD3 | gatagctgtaactcacagttaacatgctttcagtcaagtacagattgtgt |
| 1445 | AB002310 | HUWE1 | gttaaaccagatcctacggcagtccacgacccaccttgctgatgggcctt |
| 1446 | AB002333 | ZNF518 | taaagaaagccatacatagaatgcttcaagctatcttgctatgcacatta |
| 1447 | AB024402 | ING1 | agaaatgtctcgctgatgcgggagatcgacgcgaaataccaagagatcct |
| 1448 | AB028952 | SYNPO | ggtttgggacttaccggggtgatgttagatctggaacccccaagtgaggc |
| 1449 | AB032991 | NDFIP2 | cactcgacaggttgacaacctagacagctcccccggacttgccttacttt |
| 1450 | AF008915 | EVI5 | aggtcattccacatcagtttgatggtgtcccagacaagctaatccaagca |
| 1451 | AF108138 | C15orf20 | gcacccatattttaagctgctttttcaggggataaatagtgtttggggac |
| 1452 | AF116668 | LMO2 | ttaggggatggtagacctttattgggtatcaagacatagcatccaagtgg |
| 1453 | AF143327 | FLJ32810 | attttaggactctaccgaataggaggagtgaactccaaagttcaaaaact |
| 1454 | AF200348 | D2S448 | cgctgctcgaagcagctgtatagcctgtgactctccgtgtgtcagctcct |
| 1455 | AF245480 | MONDOA | tgccgtatctgctgcagacatgattgtttcttgttctagaggttttcttg |
| 1456 | AI075159 | SPTA1 | gacaaagcttggagactatgccaacctaaaacaattctaccgagaccttg |
| 1457 | BU948323 | TCF4 | gtttatacaactgtcagtccacccagtggtgcaactggttctgattcagt |
| 1458 | AI675029 | C7 | tgatgctgagagcagcaaatgtgtctgccgagaagcatcggagtgcgagg |
| 1459 | AI694012 | PSMA5 | tgttagtgttaggctattaacaatagttaggtgaaaggtaatgagggcat |
| 1460 | AI732248 | COL4A1 | acgcttacagcttttggctcgccaccatagagaggagcgagatgttcaag |
| 1461 | AJ007509 | HNRPUL1 | tcatggataagatgcgggtgatgggcctacgccggcagcggaactatgct |
| 1462 | AK022224 | ACTN1 | tgtttcccgctgtgtgtatagcgtcatgccctatgcattcccacagactc |
| 1463 | AK023928 | PSCD1 | cataagttgcttgaaagtcgagaatggcagacatagggttgtggtgttgc |
| 1464 | AK024580 | NRP1 | atcatagacgagacagtccattcatcttagttaaattggattgagaatgc |
| 1465 | AK025005 | MARCH1 | attgtctgcagtagttaaatggcacattgtactccacgattttccaaatg |
| 1466 | AK091853 | COL3A1 | gggaaacatgcataagtgccaatcctttgaatgttccacggaaacactgg |
| 1467 | AK126650 | CAPZB | ctctgtttcatgctaaccagacacgccgtgcactcgttagattcctttct |
| 1468 | AK127124 | ARHGEF4 | aaaagcaaagatgccggaagccccaaaaagcccaccctagtgagtctgcc |
| 1469 | AK127132 | DRG1 | caacccagtcctccttaccttgcacctattaacgttggttcaacatgaag |
| 1470 | AK129940 | GNAI1 | agtctttcagtacacgttactggtaagtagtttccaagttacgtgttgtc |
| 1471 | AL049313 | CLIC5 | agtctcactacttctgacactctggttattgggcactaagggccagactg |
| 1472 | AL049327 | CBLB | cactctagttcaaatccacattctagttgaatccctgcaatgtaacttga |
| 1473 | AL049449 | GAB1 | cagtgatgtgctaatacactgatcaggtttagacaatgagctttggttgt |
| 1474 | AL050391 | CASP4 | taatcaacttcaaggagcaccttcattagtacagcttgcatatttaacat |
| 1475 | AL137527 | LRCH3 | aaacatattgagtaccggttgaaagtgtctctaccttgtgatctcggagc |
| 1476 | AL353942 | SEPT11 | tgtgctttcgcctttgcatgtaagtacggtagtaagaaacctttgagatc |
| 1477 | AL833915 | TDRD9 | cccgagcacgacatggagcttgcgtttgacgttcaattcagcgtggagga |
| 1478 | AL834278 | USP30 | agcttgaactgtacggtccgtcctgcactcacccgatgcagaccttgact |
| 1479 | AU252485 | MAP7 | gacacggaccttcatggaagtccttgctctgcgtggcatctgtcagcttt |
| 1480 | AV650179 | PRG1 | ggccccttgccgattttctatacccccccacaaatccctctttttacaat |
| 1481 | AV686223 | KIAA1407 | caagtctggtgaacgaatgagccagattagtgtaatatttaggacaggaa |
| 1482 | AW014009 | MAP2K3 | tctagattagtctccaccgccgtccaggacccacttgcagcatggagtcg |
| 1483 | AW190768 | SLC13A3 | gaagttctagaaaccgcagctgacatctcgacccaagagatttaggattc |
| 1484 | AW292143 | GFAP | aactgaggcacgagcaaagtgaagacactggctcatattcctgcagcctg |
| 1485 | AW439398 | CCND1 | aacacggctcacgcttacctcaaccatcctggctgcggcgtctgtctgaa |
| 1486 | AW452039 | HRMT1L3 | gatcctcatggtctgccacgtaatcattttcttagacgtttgctccacca |
| 1487 | AW615076 | LOC492304 | acgcagactgagcatagacagagcgcagacaatcacaaagcagaaaagca |
| 1488 | AW664012 | ALOX5 | ctgatttttaagacttagaataaggaggtggggaatgtgaaatatctcat |
| 1489 | AW967479 | CD38 | gagaaccgcgacgagcaccacataagccgtaggaccgcatgaaagaagag |
| 1490 | AW970654 | PHTF2 | atgagaacttatcatgttggtgtgttaggtaaatcgggcaaatatgatag |
| 1491 | BC028066 | NALP1 | accttggcaagatatttacccaccggccatctctgtttactcatgaatgt |
| 1492 | BC034289 | LOC87769 | caacttaatggcagactgtgatcgctaactgcatctggcacaaaaacatg |
| 1493 | BC042366 | PCDH9 | cttctcatatgatagcttatgatgccggaattctgtaaacaataaagatg |
| 1494 | BC045532 | LSM8 | tagtcacattagtacataggtgtcctttgcaactgctgtcaactctacat |
| 1495 | BC050383 | TMPO | caaaagacaagttgaagagtgagttggtcgccaacaatgtgacgctgccg |
| 1496 | BC050602 | HIST1H2AC | tccctatgagaacatacagagcttcctgtacacagttgcatgtactgcat |
| 1497 | BC058855 | VEGF | ttaaacgaacgtacttgcagatgtgacaagccgaggcggtgagccgggca |
| 1498 | BE147267 | ECE1 | tgatggctatagcagaatgaggtcgggtcaggaccctcaaacaccatctg |
| 1499 | BE549606 | BZW1 | ttcaagactgtattattcaaggcttaactgaaaccggtactgatttggaa |
| 1500 | BE798965 | LAMC1 | tttcattaagtggctacatcctaacatatgcattggtcaaggttgcagca |
| 1501 | BE927772 | SFRS3 | ggtacctaagtcttgtcatggacttacaggtttgcatgggttccaaatac |
| 1502 | BF055235 | RIPK1 | caggccaattccaagtcatatgcctagtctgcataatatcccagtgcctg |
| 1503 | BF513638 | WNT3 | ctagggtctgataggaacagacctgaggcttatctttgcacatgttaaag |
| 1504 | BG682263 | HDAC3 | ctatagtgaatacttcgagtactttgccccagacttcacacttcatccag |
| 1505 | BG683220 | PMS2 | tgttcagctgccccctagtgactccgtgtgtgaagagtacggtttgagct |
| 1506 | BG747999 | MT1L | aaattccgataaagccgaggttaaccggcattaaagaacgcgggaaaaag |
| 1507 | BI493513 | PHF8 | gtgttagtaatcaaggtttagaacaccatgagatagttacccctgatctc |
| 1508 | BI559738 | GPR56 | gattacatcttctctatggcaagcgtgacttcttgctgagtgacaaagcc |
| 1509 | BM709325 | BCL7A | tatgtaacgatataagctatcggagggtgaaccgatcaggaacgctttt |
| 1510 | BM711190 | MED28 | ctttgggagggtattttgctctcgttagttttgtgggcagcctcagattc |
| 1511 | BM727177 | GOLT1B | cactgtatctctggaagtgatgcatgaattcgattggattgtgtcatttt |
| 1512 | BQ009245 | GPM6B | ctccttaatagggacctgtgactccttaataaggacctgtgacatgccca |
| 1513 | BQ021469 | AGPS | agttcagtactgtaggaggatgggtatctactcgcgcatcaggcatgaag |
| 1514 | BQ215285 | LEF1 | ggcgtaggagtaacctcgcactttgctagctaacctgactccgggtgagg |
| 1515 | BQ898221 | ITGA6 | attagaattagagcatgggagggtcatcactatgacctaaattatttact |
| 1516 | BU929213 | SESN3 | gctgtagatatagtctaaggtagttaaccatataagccttttcaactctt |
| 1517 | BU954396 | RPL11 | ttagccattgctgcaatctctgctgttgcctcctgttctgaaaaaattaa |
| 1518 | BX095432 | CNTN5 | gtgtaaagataaatgatctgcttattgatagtgtcatcgaatgagcatga |
| 1519 | BX640908 | EVI1 | gtagcatggctctttcatataggactatttacaagactgctgagcagaat |
| 1520 | BX648339 | USP53 | taatggttctactaatttggactcacctgttatcgatggaaatggtacag |
| 1521 | BX648365 | SEPT7 | atggagattaagatgccttgaattgtctagggtgttctgtacttagaaag |
| 1522 | BX649193 | TKT | atggtggcgtccctgaggtagtgtagggtggaagctatggtgcagcttgg |
| 1523 | C05886 | ETV6 | gcgtgactcatttcaacactaagtactaggggtgttgtcaggagacaaat |
| 1524 | D31762 | TRAM2 | catggatgctttaattggaagtgggttgccgacccccttgtgcctagaaa |
| 1525 | D86967 | EDEM1 | ggtgaaaaggtagggctgagtgattaccttagccacagggtggctgagca |
| 1526 | 1141942 | NPEPPS | aatggttaaacacaaaaggctgatagctggtatcacatagttggagtcag |
| 1527 | M60028 | HLA-DQB1 | cattcgaatactaaaatagtgggagtggtctttttcgtcatccctgcggc |
| 1528 | M77810 | GATA2 | tctcctctggcttccctctctgaaatagccgaactccaggctgggctgag |
| 1529 | N26272 | MSI2 | atttactaacatatgataggtattaagccactagggtcatgcctgacccc |
| 1530 | N28431 | COL6A1 | caccaaggaccgcctgggccggacgaatgcgagattttggacatcatcat |
| 1531 | N41620 | WWP1 | tgttcagaaaggtattaagggcttaggccaaatcttactttgagtatgtt |
| 1532 | N53163 | COL27A1 | catcagggaagcagtaccgcctggaagttggacctgcgtgcttcctctga |
| 1533 | N54946 | EWSR1 | ttcagtgtcttgtacaggtaaggcactcaatgttgttaacatgccctttt |
| 1534 | N70000 | UBE3B | tcaggtaaggcctcgccaagcctctatgcaccccacaaagtttctgcctc |
| 1535 | NM_000016 | ACADM | atgataaaactggtgaatatccagtccccctaattagaagagcctgggaa |
| 1536 | NM_000022 | ADA | gccttcgacgagcccaaagtagaactgcatgtccacctagacggatccat |
| 1537 | NM_000024 | ADRB2 | ttcccccactcctcttatttgctcacacggggtattttaggcagggattt |
| 1538 | NM_000033 | ABCD1 | tccctattcgtagccatctccgtggccaatgtgactaccgtgccagcagc |
| 1539 | NM_000062 | SERPING1 | gacagaggacccagatcttcaggtttctgcgatgcagcaccagacagtgc |
| 1540 | NM_000089 | COL1A2 | catgagtgtgatccacattgttaggtgctgacctagacagagatgaactg |
| 1541 | NM_000099 | CST3 | gaaaccctcaaataggccagaacaactcactaattaaattcatcccctcc |
| 1542 | NM_000110 | DPYD | cattaatattggtggattgcagcaatttgctactgaggtattcaaagcaa |
| 1543 | NM_000118 | ENG | aagtatgatcagcaatgaggcggtggtcaatatcctgtcgagctcatcac |
| 1544 | NM_000120 | EPHX1 | tattcctacatggttcgtgggggccactttgcggcctttgaggagccgga |
| 1545 | NM_000147 | FUCA1 | attttcagagcaatctaaaagcgccaaaattcgctatgtttacagtgata |
| 1546 | NM_000169 | GLA | ccggcaggagattggtggacctcgctcttataccatcgcagttgcttccc |
| 1547 | NM_000182 | HADHA | atttatcagagtaacgagaagacaaactccggcactgggtttgctccctg |
| 1548 | NM_000194 | HPRT1 | ttattcctcatggactaattatggacaggactgaacgtcttgctcgagat |
| 1549 | NM_000214 | JAG1 | gccccatctaggagagccttgattcccttgttattcaacagcaagtgtga |
| 1550 | NM_000234 | LIG1 | aagcagacgttctgcgaggttcccgacctggaccgaattatccccgtgct |
| 1551 | NM_000239 | LYZ | aaatgggagagtggttacaacacacgagctacaaactacaatgctggaga |
| 1552 | NM_000246 | MHC2TA | gtcagagcctcgagttattaacactaacgatttgttaatgatagctactg |
| 1553 | NM_000249 | MLH1 | caaagtgtaccaacataagtgttggtagcacttaagacttatacttgcct |
| 1554 | NM_000251 | MSH2 | cctctacctacgatggatttgggttagcatgggctatatcagaatacatt |
| 1555 | NM_000277 | PAH | accagacggtcgcaggcttagtccaattgcagagaactcgcttcccaggc |
| 1556 | NM_000305 | PON2 | agctgctcataggcactttataccacagagccttgtattgtgaactctaa |
| 1557 | NM_000341 | SLC3A1 | tgctgcttggtgaacaatcattaattcttcgatatttctgtagcttgaat |
| 1558 | NM_000361 | THBD | acaacaacaccagctatagcaggtgggcacggctcgacctcaatggggct |
| 1559 | NM_000382 | ALDH3A2 | gacagatggtggatggtctggacaagaagcgagtaagccactgcggttgg |
| 1560 | NM_000395 | CSF2RB | cccttgagaccccacgtcatgtagagaagttaacggcccaagtggtgggc |
| 1561 | NM_000417 | IL2RA | ataatctttgggtaacttcaaaacggggcagccaaacccatgaggcaatg |
| 1562 | NM_000418 | IL4R | gccagacccccaaaatcgtgaactttgtctccgtgggacccacatacatg |
| 1563 | NM_000435 | NOTCH3 | agtacacgacccaggtatgtaccaagtaggcacccttgggcgcacccact |
| 1564 | NM_000448 | RAG1 | acttatgagttggtttttgcaattgagtttccctctgggttgcattgagg |
| 1565 | NM_000484 | APP | cttccacataggaaagactttgacccaatgaggaattagtcattgtcctt |
| 1566 | NM_000485 | APRT | tgcttcttgttccttctgcgagctggtattgagcgcctgccacgagccag |
| 1567 | NM_000536 | RAG2 | atataccttcaagcaaccctttggattatgcccatgaacaagttagtttc |
| 1568 | NM_000545 | TCF1 | aacctggccttcagtgtaccgcgtctaccctgggattcaggaaaaggcct |
| 1569 | NM_000546 | TP53 | gggcagctggttaggtagagggagttgtcaagtcttgctggcccagccaa |
| 1570 | NM_000561 | GSTM1 | taagaagaactgtgcggcacagcgcccgctactacagctcgtcacacggt |
| 1571 | NM_000574 | DAF | aagtggaaccacttcaggtactacccgtcttctatctgggcacacgtgtt |
| 1572 | NM_000578 | SLC11A1 | cctcaggatttgcggagcctgtaataattgagggacaggcggggcgccat |
| 1573 | NM_000594 | TNF | gtgtctgtaatcgccctactattcagtggcgagaaataaagtttgcttag |
| 1574 | NM_000598 | IGFBP3 | gactttgtgacttaggcggctgtgttgcctatgtagagaacacgcttcac |
| 1575 | NM_000601 | HGF | ttgatcaactatgatggcctattacgagtggcacatctctatataatggg |
| 1576 | NM_000604 | FGFR1 | ggtggtgccagtggcttattaattccgatactagtttgctttgctgacca |
| 1577 | NM_000610 | CD44 | ttgtagtccctcacttggatatacctctgttttcacgatagaaataaggg |
| 1578 | NM_000628 | IL10RB | cactaaaataagatcatgttttaattgtgagaaacagggccgagcacagt |
| 1579 | NM_000629 | IFNAR1 | gatgttacagttcctaatttgaaaccactgactgtatattgtgtgaaagc |
| 1580 | NM_000640 | IL13RA2 | tatcaagagacatggtattgactcaacagtttccagtcatggccaaatgt |
| 1581 | NM_000660 | TGFB1 | ggataacacactgcaagtggacatcaacgggttcactaccggccgccgag |
| 1582 | NM_000700 | ANXA1 | gaactcgccataaggcattgatcaggattatggtttcccgttctgaaatt |
| 1583 | NM_000701 | ATP1A1 | agaacggcttcctcccaattcacctgttgggcctccgagtggactgggat |
| 1584 | NM_000717 | CA4 | agaaggtcgtctggactgtgttccgggagcccattcagcttcacagagaa |
| 1585 | NM_000729 | CCK | aaaggcactgccaaccccatttcacagaccgcaaaccgaggcgcgaggaa |
| 1586 | NM_000732 | CD3D | gtaaatgtggttgcattgtcaatagggacgctaaagttcaggccaccttt |
| 1587 | NM_000773 | CYP2E1 | atggacggtatcaccgtgactgtggccgacctgttctttgcggggacaga |
| 1588 | NM_000791 | DHFR | tcaggggttacctccatgtgccctttttagggaagctgttggagaaaata |
| 1589 | NM_000801 | FKBP1A | caccggacggagaacatgatgaacgacgcacatggcatagcaacgacgaa |
| 1590 | NM_000852 | GSTP1 | tggtgccggggcgacaaggtcgcaactataatctaaccgccttgctgctc |
| 159 | NM_000856 | GUCY1A3 | attagaagattttacaggacgagggctctacctctcagacatcccaattc |
| 1592 | NM_000867 | HTR2B | aactctcagaaaacgctccagtaagatctacttccggaatccaatggcag |
| 1593 | NM_000878 | IL2RB | tatctatacatccgtatcttttaatgatccgtgtgtaccatctttgtgat |
| 1594 | NM_000885 | ITGA4 | actgtagcaatacctttaaaatatgaggttaagctgactgttcatgggtt |
| 1595 | NM_000887 | ITGAX | ccaaaatgtcgtgcaccgattgttccatgcctcatatggggcccgtaggg |
| 1596 | NM_000905 | NPY | ttaaccttggtcagcactcggcacacccctgcgcggtgcacataacagca |
| 1597 | NM_000906 | NPR1 | tgaaacgtgtgaaccgtaaacgcattgagctgac&cgaaaagtcctgttt |
| 1598 | NM_000953 | PTGDR | acctccagccagaagtcccttccaaatataagagtactcatgtttattta |
| 1599 | NM_000960 | PTGIR | gtgggtgatgacgtaggacatgtgcttggtacaaaaagggcctgagacat |
| 1600 | NM_000964 | RARA | gggcaaatacactacgaacaacagctcagaacaacgtgtctctctggaca |
| 1601 | NM_000967 | RPL3 | tgataactgcaaaggggagcttggaattatgttagtcagtccctgtgaat |
| 1602 | NM_000968 | RPL4 | gggcttttcaagattctgctaagatctgtattaatcatcttgtgcatggg |
| 1603 | NM_000969 | RPL5 | ttctgccacatcacgcagagctagtgatcgagtttgcacaattacaccaa |
| 1604 | NM_000970 | RPL6 | gtgaaccctccacggtggaaaaaatgccgcccttagtcgggtggggaaaa |
| 1605 | NM_000972 | RPL7A | gctggcttaacctatgagaagttctatctgacgatcagcttggaacagcc |
| 1606 | NM_001001547 | CD36 | gtatgcacgtacaaatttcttaacctgttatcaatgtctgagctacataa |
| 1607 | NM_001005 | RPS3 | gcgtatcagaggtagcggagaatgaagagcgacaagtgggacgagctgta |
| 1608 | NM_001009 | RPS5 | aggaagacgagacgactagaagcacacaataagacgacaaccagacacaa |
| 1609 | NM_001015 | RPS11 | tagcccatatattcatgtgtcatcgttcaggaacaagtcagtgacaaact |
| 1610 | NM_001019 | RPS15A | cgcacatttgcatactatatgcttgttacagtgatccccacagtaactca |
| 1611 | NM_001069 | TUBB2 | caatactttaattgtaagaggagttccacatcattacatcaacagtgtga |
| 1612 | NM_001079 | ZAP70 | agtaacggtgccacgtggataccaatgcacacgcccctagagtccaccct |
| 1613 | NM_001092 | ABR | tctgctgatacacaggtaggatgggacccttcatgaatatctgactttaa |
| 1614 | NM_001130 | AES | aatgctgtatcaagggtgggcttagctgtgcctttccaataaagatgtga |
| 1615 | NM_001150 | ANPEP | tggtgaaggacagccagtatgagatggacagcgagttcgagggggagttg |
| 1616 | NM_001165 | BIRC3 | ccttgatgagaagttcctaccactgtgcaatgaataacgaaaatgccaga |
| 1617 | NM_001175 | ARHGDIB | tctcccccaactgatatgttcatagaatgacagtttgtagatagaagatc |
| 1618 | NM_001178 | ARNTL | gcaaaactctctcattgaaagcgttcacatagttcctaatgtttctttat |
| 1619 | NM_001207 | BTF3 | tattctttggttctgctcccccactattgaccaatgtatgagatgggaag |
| 1620 | NM_001211 | BUB1B | tgtattgtttggcaccaatatataaactgcttcacccttcaggatcttct |
| 1621 | NM_001219 | CALU | tgagaagaattatcgtattccacgtttttagccctcaggttaccaagata |
| 1622 | NM_001226 | CASP6 | gatagagacaatcttacccgcaggttttcagatctaggatttgaagtgaa |
| 1623 | NM_001228 | CASP8 | ggaggagttgtgtggggtaatgacaatctcggactctccaagagaacagg |
| 1624 | NM_001229 | CASP9 | accagtatcgctcatagatcagcaaaccggggcctactagagtctgaaca |
| 1625 | NM_001237 | CCNA2 | gactggatataccctggaaagtcttaagccttgtctcatggaccttcacc |
| 1626 | NM_001239 | CCNH | ttgaactaaatatccagcagatacaacaagtagtcagaagttcaaagatg |
| 1627 | NM_001255 | CDC20 | cgcaaagaggcaagcgcaagacgacaggaaacgaagcgagagacgagaag |
| 1628 | NM_001256 | CDC27 | tcccataaacaaatgtaatgggcatattgggactcgtatgtaggaatcaa |
| 1629 | NM_001265 | CDX2 | ttaaagccttctggatccatggggggagaagtgatatggtgaagggaagt |
| 1630 | NM_001266 | CES1 | ggaggagcaaagactggggtcttttgcgaaagggattgcaggttcagaag |
| 1631 | NM_001273 | CHD4 | aggatgcttttactacccagtggcttgtaagagacctgcgaggcaaatca |
| 1632 | NM_001274 | CHEK1 | atattggttgacttccggctttctaagggtgatggattggagttcaagag |
| 1633 | NM_001280 | CIRBP | gtgcgccccacagtagacgtgcagacgtccctgagaggttcttgaagatg |
| 1634 | NM_001295 | CCR1 | ggaacatagaactcatgacggaagagttgagacctaacgagaaatagaaa |
| 1635 | NM_001312 | CRIP2 | agtgttatttatgctcccttcgtgggtgatggccacgccctcaccatgtc |
| 1636 | NM_001313 | CRMP1 | ataaggatgtctaccaaatgtccgacagccagctctatgaagcctttacc |
| 1637 | NM_001337 | CX3CR1 | agcactcccaagtctacagagtgatagccttccgtaacccaactctcctg |
| 1638 | NM_001344 | DAD1 | gaccggagtaccttgcgtgcagttatgtcggcgtcggtagtgtctgtcat |
| 1639 | NM_001345 | DGKA | gaccgggctgcattgtgtatggtgccacctagagatccacgatgactgcc |
| 1640 | NM_001398 | ECH1 | ctgcagacccaagacctcgtgaagtcggtccaggccacgactgagaacaa |
| 1641 | NM_001404 | EEF1G | attggaccgacataggaccgtccggatgcaactctccccgtacaaccctg |
| 1642 | NM_001428 | ENO1 | aagtgtctagagtcatgtgagcctcgtgtcatctccggggtggccacagg |
| 1643 | NM_001436 | FBL | ctaggaccctcacgaacggacaacacagaagcgcaacgcgcaacacacaa |
| 1644 | NM_001441 | FAAH | gcagcccatgggtatgacataggccaaggcccaactaacagtcaagaaac |
| 1645 | NM_001456 | FLNA | aggcaacctgacggagacctacgttcaggaccgtggcgatggcatgtaca |
| 1646 | NM_001459 | FLT3LG | ccgtgagctgtctgactacctgcttcaagattacccagtcaccgtggcct |
| 1647 | NM_001462 | EPRL1 | tgtaatggtctctgaaaaggaattgagaagtaattcctctgattctgttt |
| 1648 | NM_001489 | NR6A1 | tcacagatgccttctctaagacagctcctgtgtacagcatcgttctttgc |
| 1649 | NM_001513 | GSTZ1 | ctacgagacggtgcccatcaatctcataaaggatgggggccaacagtttt |
| 1650 | NM_001515 | GTF2H2 | tataatggagaaagattttgttatggatgtcagggggaattgaaagacca |
| 1651 | NM_001540 | HSPB1 | tttaccctcgcgcgctgattatccaccctgggcgagggataccgatgtgt |
| 1652 | NM_001560 | IL13RA1 | taccagtcccgacactaactatactctctactattggcacagaagcctgg |
| 1653 | NM_001607 | ACAA1 | atgggagccgctgccgtctttgaataccctgggaactgagtgaggtccca |
| 1654 | NM_001630 | ANXA8 | gaaacatcgtttcaaggagcgagattgacttaaatcttatcaaatgtcac |
| 1655 | NM_001640 | APEH | ctgagatgctggacaaatcgcccatcagatacatccctcaggtgaagaca |
| 1656 | NM_001654 | ARAF | gacgtctatgcctacggggttgtgctctacgagcttatgactggctcact |
| 1657 | NM_001664 | RHOA | cccaccccagagctatgccaacaaaatctgttacggagtaaagccctgaa |
| 1658 | NM_001700 | AZU1 | ggctgagaaacagtagctatcaaacgccggctgtgagccacgtctgtgct |
| 1659 | NM_001712 | CEACAM1 | aggaggattagcttggagttctctatactcaggtacctctttcagggttt |
| 1660 | NM_001715 | BLK | caccttactggggcttagtactccggatgaccgtgcgaggtcactgttac |
| 1661 | NM_001716 | BLR1 | ataagctatagacccgaggaaactcagagtcggaacggagaaaggtggac |
| 1662 | NM_001728 | BSG | cactcccagtgcttgcaagattccaagttctcacctcttaaagaaaaccc |
| 1663 | NM_001734 | C1S | aatttagctatagtctagaaagttagtcgaagttaactagggagggtgtg |
| 1664 | NM_001743 | CALM2 | actattgcaaaacgggtgtattatccaggtactcgtacactatttttttg |
| 1665 | NM_001747 | CAPG | agactgcagaggaatgacgaaagcgacgggccagttgacgacgcgaagag |
| 1666 | NM_001752 | CAT | caggaaagtacccctcctggactttttacatccaggtcatgacatttaat |
| 1667 | NM_001753 | CAV1 | cgctttattggctgagatatgaacatattgttgaaaggtaatttgagaga |
| 1668 | NM_001759 | CCND2 | cagcagtaccgtcaggaccaacgtgacggatccaagtcggaggatgaact |
| 1669 | NM_001767 | CD2 | agccacacatctccataaagatgtaagaggtaattatttcagctgggtgc |
| 1670 | NM_001772 | CD33 | caatgcatatcattcagacataaagatgggccgaagtacagatacatgct |
| 1671 | NM_001774 | CD37 | tcttctgtgtggtgagtggaccgcttaccccactaggtgaagatgtcagc |
| 1672 | NM_001776 | ENTPD1 | attcaacaatctaaccatactgagctgctgattccccagggattgcatat |
| 1673 | NM_001783 | CD79A | ctaggtcgcacagggcatagacgatagtaccattcctggttgaaacacga |
| 1674 | NM_001786 | CDC2 | tctccagaagtattgctggggtcagctcgttactcaactccagttgacat |
| 1675 | NM_001793 | CDH3 | atgacgtggcaccaaccatcatcccgacacccatgtaccgtcctaggcca |
| 1676 | NM_001794 | CDH4 | acttggtgctatgagccctccaagtattaaccttacacagagcccccgct |
| 1677 | NM_001795 | CDH5 | ctgcatgacggagccgagccatgtgtctttacacctcgctgttgtcacat |
| 1678 | NM_001798 | CDK2 | agccttcctacacgttagatttgccgtaccaatctctgaatgccccataa |
| 1679 | NM_001830 | CLCN4 | ttaggtccaccatacgatctgtgtaagtaagattatcccacttgcccttt |
| 1680 | NM_001838 | CCR7 | tgcaaggggcgtgggagtggcctgaagagtcctctgaatgaaccttctgg |
| 1681 | NM_001839 | CNN3 | gtatcaatgtagagttgctctgttttcttcaactgtatttattgctgcat |
| 1682 | NM_001897 | CSPG4 | actggctaggggtgtcaatgcatcagccgtagtgaacgtcactgtgaggg |
| 1683 | NM_001901 | CTGF | tgagagtgtgaccaaaagttacatgtttgcacctttctagttgaaaataa |
| 1684 | NM_001905 | CTPS | ttactttcttaatgtgacctagcaataggcatagctacgtggcactatat |
| 1685 | NM_001908 | CTSB | cttaacaaaggttaccataagccacaaattcataaccacttatccttcca |
| 1686 | NM_001916 | CYC1 | aacacgacagccaacgtgcataagcatgcaacactaccgaggtagccaca |
| 1687 | NM_001923 | DDB1 | gcaaacctacagtatgacgatggcagcggtatgaagcgagaggccactgc |
| 1688 | NM_001928 | DF | agtagtgattagtcgtatttcgcgacacctcacacggtagatgtgatcaa |
| 1689 | NM_001939 | DRP2 | ctcttgatggagccagatccccatcctatagttcatagtcctctcctggt |
| 1690 | NM_001941 | DSC3 | gaatataccattcctattactgtaaaagacagggccggccaagctgcaac |
| 1691 | NM_001952 | E2F6 | ccaaggggttttacttaggacaagttgtaccttgccctctctccagctct |
| 1692 | NM_001964 | EGRL | taaacacattgaatgcgctttattgcccatgggatatgtggtgtatatcc |
| 1693 | NM_001993 | F3 | gattatatattccgcacttaaggattaaccaggtcgtccaagcaaaaaca |
| 1694 | NM_001999 | FBN2 | atgtgctattaagagcacgtatccattattcttctcaacccaagaacctg |
| 1695 | NM_002015 | FOXO1A | acccgagtttagtaacagtgcagattccacgttcttgttccgatactctg |
| 1696 | NM_002017 | FLI1 | gaaagcaaagcactggtacagttatttaacaggcatacacaagcagggaa |
| 1697 | NM_002023 | FMOD | tacccgtatgagacctacgagccttacccctatggggtggatgaagggcc |
| 1698 | NM_002033 | FUT4 | ctcctagttccacgtgctatcattctactaactggcaccctaaggttgaa |
| 1699 | NM_002046 | GAPD | aaaagcccctttggtgtgaaaaacggcccccacaagtttataaaaggggg |
| 1700 | NM_002049 | GATA1 | ccaagaagcgcctgattgtcagtaaacgggcaggtactcagtgcaccaac |
| 1701 | NM_002064 | GLRX | cttctggcgtaaaaacattctcgagttaataatgttaccccttataacag |
| 1702 | NM_002065 | GLUL | aagtcaaataactcctcattgtaaacaaactgtgtaactgcccaaagcag |
| 1703 | NM_002067 | GNA11 | ttattgacgcccagcgcgcctcgcctcttcacccatcaacgctgtgcttt |
| 1704 | NM_002068 | GNA15 | ggagcatggactttggaaccagaattctaggtgcgagtctcagctgtgtg |
| 1705 | NM_002076 | GNS | tcttacagttgatgcaaagtatcaactactttaccctaccttctcccctt |
| 1706 | NM_002086 | GRB2 | ttaatcctttaactctgcggatagcatttggtaggtagtgattaactgtg |
| 1707 | NM_002090 | CXCL3 | gatgttcttgaggtgaattccctgcagtgtctgcaagcactggcagcgca |
| 1708 | NM_002094 | GSPT1 | cttagtgactagttttcgctagacagcaggttaatacctagatctcattt |
| 1709 | NM_002102 | GYPE | gaagtgggatttacccattaccatatattcaccaccaggtcactgacgag |
| 1710 | NM_002107 | H3F3A | gcgaggacggggctttgtagaatgctcgctggtggtagctgttgttcctc |
| 1711 | NM_002108 | HAL | ggtgcgctctgttgtaaggccctggataaaagatcgcttcatggccccgg |
| 1712 | NM_002110 | HCK | caatccacaatctgacattctcaggaagcccccaagttgatatttctatt |
| 1713 | NM_002118 | HLA-DMB | cacatgggagatctagatgtagaaggtacacaagtagtaggataactcac |
| 1714 | NM_002121 | HLA-DPB1 | cagactgcaaatctgcctgataggacccatattcccacagcactaattca |
| 1715 | NM_002128 | HMGB1 | ctgtgcaaaggttgagagctattgctgattagttaccacagttctgatga |
| 1716 | NM_002129 | HMGB2 | ggtagcacagcaaacttgtaggaattagtatcaatagtaaattttgggtt |
| 1717 | NM_002135 | NR4A1 | ttggggccctagtggtggggctacaaggcttgctcctagatctactccaa |
| 1718 | NM_002141 | HOXA4 | tgtcagcgccgttaaccccagttataacggaggggagcctaagcgctctc |
| 1719 | NM_002145 | HOXB2 | tccttccaatgaaatctcaggaataattaaactctagggggactttctta |
| 1720 | NM_002162 | ICAM3 | aagaagctgcgtccgtcgtcactctcggtagcatttagctgaagttgagc |
| 1721 | NM_002166 | ID2 | ttggacctgcagatcgccctggactcgcatcccactattgtcagcctgca |
| 1722 | NM_002167 | ID3 | ctatagaggccgattatcgaagccggtacgtaaagacagcgcaagaagag |
| 1723 | NM_002186 | IL9R | gaacctgtcgtgcaaagctcacgtcaccaactgctgcagttatctcctga |
| 1724 | NM_002193 | INHBB | cacgtgaactatgcaatttaaagggttgacccacactagacgaaactgga |
| 1725 | NM_002198 | IRF1 | taaatgaatctcgttcccaaagaactacccccttttcagctgagccctgg |
| 1726 | NM_002199 | IRF2 | ctgaaacctatcgaataatcgggccgagccgcagagcacaagggaaccag |
| 1727 | NM_002222 | ITPR1 | agggaactttctatgcaatgttcaggataaatcgatactgctggccaatc |
| 1728 | NM_002224 | ITPR3 | attgtgctggtccgcgtgaagaacaagaccgactacacgggccctgagag |
| 1729 | NM_002228 | JUN | ttgcggccccgaaacttgtgcgcgcagcccaaactaacctcacgtgaagt |
| 1730 | NM_002229 | JUNB | tacacgactacaaactcctgaaaccgagcctggcggtcaacctggccgac |
| 1731 | NM_002230 | JUP | gccagtgatagtagttaccttgcagggtcgttgtcaggttatgtgagaaa |
| 1732 | NM_002248 | KCNN1 | aagacccagaccgtcatgtacgaccttgtatcggagctgcacgctcagca |
| 1733 | NM_002276 | KRT19 | acaggaagatcactacaacaatttgtctgctccaaggtctctgaggcagc |
| 1734 | NM_002295 | LAMR1 | gtaggtaggttaacataagatgcctccgtgagaggctggtggtcagccct |
| 1735 | NM_002312 | LIG4 | catattgaaatgtacggaatttgatccatgcgatactatgtgtgcattat |
| 1736 | NM_002341 | LTB | ggcagcatgagtggttaagagtcaagggagtagccggggtagtggcttac |
| 1737 | NM_002349 | LY75 | ttgtaaaacgtgatggatttccactatgggttgggctctcaagtcatgat |
| 1738 | NM_002351 | SH2D1A | tccccgtgtgtaggtagatctggtctttagaggcagatagataggtcagt |
| 1739 | NM_002353 | TACSTD2 | tgttaactgggcctatgtagtagcctcatttaccatcgtttgtattactg |
| 1740 | NM_002356 | MARCKS | taggaacttttcacttatctcatgttagctgtaccagtcagtgattaagt |
| 1741 | NM_002379 | MATN1 | gaagaagttcatcagtcagatcgtggatacgctggacgtgtcagacaagc |
| 1742 | NM_002388 | MCM3 | gctgccaaccctgtctacggcaggtatgaccagtataagactccaatgga |
| 1743 | NM_002390 | ADAM11 | ccacatgtcccagatcgtctccaattcgaaaacaaccgtcctgctgtccc |
| 1744 | NM_002392 | MDM2 | aagttgaatctctcgactcagaagattatagccttagtgaagaaggacaa |
| 1745 | NM_002393 | MDM4 | tagtatgggtttctaatcctaggcttgtacaatggattggagttgagcca |
| 1746 | NM_002398 | MEIS1 | gcgcgcatggctgaaatcctaggcgagaagaaagattcttctgcctgata |
| 1747 | NM_002412 | MGMT | atgtttgagcgacacacacgtgtaacactgcatcggatgcggggcgtgga |
| 1748 | NM_002414 | CD99 | caaagagcccgcagcgatgaaacgagactgaggcggcgaacaaacagaaa |
| 1749 | NM_002415 | MIF | ttttccccgcaccccctaataatcgctcgccttatatgtcccccccgcac |
| 1750 | NM_002417 | MKI67 | tattggctgccatgatagggttctcacagcgtcatccatgatcgtaaggg |
| 1751 | NM_002419 | MAP3K11 | tatcagcatgccactcgacttcaagcaccgcatcaccgtgcaggcctcac |
| 1752 | NM_002421 | MMP1 | cgtgtgacagtaagctaacctttgatgctataactacgattcggggagaa |
| 1753 | NM_002422 | MMP3 | ttttatgttattacagggcattcaaatgggctgctgcttagcttgcacct |
| 1754 | NM_002436 | MPP1 | tctctgcagctatcctaattcagccagtaaggttcagtcttcttgctcag |
| 1755 | NM_002441 | MSH5 | ctcgtggcaaggaggtctcagacttgatccgcagtggaaaacccatcaag |
| 1756 | NM_002444 | MSN | tcctctccagctataacagtagggatgagtacccaaaagctcagccagcc |
| 1757 | NM_002447 | MST1R | gagatgaatgtgcgtccagaacagccgcagttctcacccatgccagggaa |
| 1758 | NM_002460 | IRF4 | catctgttctatgcttcctcgtgccaattatagtttgacagggccttaaa |
| 1759 | NM_002462 | MX1 | tcctgtcggagccctgtctcctctctctgtaataaactcatttctagcag |
| 1760 | NM_002466 | MYBL2 | ttagtggttgtgaatggtgcgtgtgagatctgcatcctggcgtcaggcct |
| 1761 | NM_002475 | MLC1SA | tcatgacacgcgcccaccctatgcgcgttttcacaaactccgaagaacgc |
| 1762 | NM_002483 | CEACAM6 | actcttggtattaccctcctaatagtcatactagtagtcatactccctgg |
| 1763 | NM_002512 | NME2 | ctgaacacgtgcggacaacgacacgcttcgcgcgctgttggatgtaacca |
| 1764 | NM_002514 | NOV | agggagataactgtgtgttcgatggggtcatctaccgcagtggagagaaa |
| 1765 | NM_002527 | NTF3 | aataaactcgtgggctggcggtggatacggatagacacgtcctgtgtgtg |
| 1766 | NM_002530 | NTRK3 | gaggtcattgagtgcattacccaaggtcgtgttttggagcggccccgagt |
| 1767 | NM_002572 | PAFAH1B2 | tcgccgtagacagcgctctggctaccaccgtgaggctacttgaactgtca |
| 1768 | NM_002576 | PAK1 | ccttctcatgctctgaggtactactgcctctgcagcacaaatttctattt |
| 1769 | NM_002585 | PBX1 | ctgcccaagcaaatgcattataatcataagtgatccagtatttcttctct |
| 1770 | NM_002592 | PCNA | gatatcattacactacgggccgaacgatcacgcggataccttggcgctag |
| 1771 | NM_002600 | PDE4B | gttcaggcgttcttctcctagacaactataccgatcgcattcaggtcctt |
| 1772 | NM_002608 | PDGFB | ttccctcgtccgtctgtctcgatgcctgattcggacggccaatggtgctt |
| 1773 | NM_002609 | PDGFRB | agtgggttctcaatacggtaccaaagatataatcacctaggtttacaaat |
| 1774 | NM_002616 | PER1 | atcaggacgcactttccggctccagtgacctgctcgaacttctgctgcaa |
| 1775 | NM_002619 | PF4 | gggaggtggtcttcacacacaggcactgcaggtccccatcttcttcagct |
| 1776 | NM_002621 | PFC | ttcatgtgtcattgactccgaccttcagttacccatgcttttagctgcca |
| 1777 | NM_002648 | PIM1 | ttctgctgaatgccgcgatgggtcaggtaggggggaaacaggttgggatg |
| 1778 | NM_002658 | PLAU | aatttcagtgtggccaaaagactctgaggccccgctttaagattattggg |
| 1779 | NM_002659 | PLAUR | ccatgaatcaatgtctggtagccaccggcactcacgaaccgaaaaaccaa |
| 1780 | NM_002687 | PNN | tccgacagaaagaggtctatatcagagagtagtcgatcaggcaaaagatc |
| 1781 | NM_002704 | PPBP | gaagtgatcgggaaaggaacccattgcaaccaagtcgaagtgatagccac |
| 1782 | NM_002729 | HHEX | taagtaacttgactataaaataaagccgtccgtgggacgactgacctcgt |
| 1783 | NM_002730 | PRKACA | gggcgattcaacctgtgtgctgcgaaggacgagacttcctcttgaacagt |
| 1784 | NM_002744 | PRKCZ | tgaccatctgtggtaaccgagaattccttggaggcagcgtcttcaccgcc |
| 1785 | NM_002753 | MAPK10 | atgcactggggtaccacatggtccatttcatgtgatctattactctgaca |
| 1786 | NM_002758 | MAP2K6 | gttggaaaagaatcatgggtctcaagatcaaatgaattggaatttgcatt |
| 1787 | NM_002791 | PSMA6 | agtgcttctaccaacatgtcccgtggttccagcgccggttttgaccgcca |
| 1788 | NM_002800 | PSMB9 | aatatcagctataaatatcgagaggacttgtctgcacatctcatggtagc |
| 1789 | NM_002814 | PSMD10 | ggattctgtaatgttcctccatacagttaaaacatcctaacttgtttttc |
| 1790 | NM_002827 | PTPN1 | ttgcttacttcccccttctaaacaacaatcccaacccttacgccatggtg |
| 1791 | NM_002831 | PTPN6 | cacaacctgaacctaggagtgccccattcttttgtaatttaaatggctgc |
| 1792 | NM_002835 | PTPN12 | ccacaggaattaagttcagatctaaatgtcggtgatacttcccagaattc |
| 1793 | NM_002844 | PTPRK | agaaccagattgaaatgagaacgtattggtgtttgtacagtgaacatgcc |
| 1794 | NM_002856 | PVRL2 | ctagggttccagactggttggacttgttcgtctggacgacactggagtgg |
| 1795 | NM_002868 | RAB5B | ccaacataacaatcgtggtaacagaatgcgactgctgatttaccgatgta |
| 1796 | NM_002870 | RAB13 | aggttttagggtcctgcaaaaggctagctcggcactacactagggaattt |
| 1797 | NM_002880 | RAF1 | ttgagctgctccaacactctctaccgaagatcaaccggagcgcttccgag |
| 1798 | NM_002887 | RARS | agcagatatgattatctatgttgtggacaatggacaatctgtgcacttcc |
| 1799 | NM_002893 | RBBP7 | gcactttcaagtgagctgttgcgtactgtatcatattgtagctattaggg |
| 1800 | NM_002901 | RCN1 | catgtttgtcggaagccaagctaccaattacggggaagatctcacaaaaa |
| 1801 | NM_002908 | REL | tgtgcagataacagcatgataaatgagtcgggaccatcaaacagtactaa |
| 1802 | NM_002913 | RFC1 | agcagtaggcttatgtacacctcttatagaggttgataggactgcttggg |
| 1803 | NM_002934 | RNASE2 | agtaacaaaactcgcaaaaattgtcaccacagtggaagccaggtgccttt |
| 1804 | NM_002935 | RNASE3 | gacactatagagtgtgtcataaccgagaccggataggggagtagttactt |
| 1805 | NM_002936 | RNASEH1 | tgtctgtcaaggaaggtgagtggtgaagggtttttacatatatacaagaa |
| 1806 | NM_002953 | RPS6KA1 | gaactcattctatccccaatcagctccttttccgttctgttctgctggga |
| 1807 | NM_002960 | S100A3 | ccccgactgagtttcgggaatgtgactacaacaaattcatgagtgttctg |
| 1808 | NM_002961 | S100A4 | atgactgctgtccgcgactactcgcgttctgatgacacgtctactaaaac |
| 1809 | NM_002971 | SATB1 | gaggcgaggtggccttcccaatgcgcgttattcggggttattgaagaata |
| 1810 | NM_002972 | SBF1 | cttccgcatttctccggtcaaccgcatgtatgccatctgccgcagctacc |
| 1811 | NM_002975 | CLEC11A | ctgcgtgctgtgcgactgtttttcgatccgctaggtgtggctgcgactgt |
| 1812 | NM_002983 | CCL3 | tgcatcacgtgagtctgagtttcgttgtgggtatcaccactctctggcca |
| 1813 | NM_002984 | CCL4 | gtatttctccccgtccgggcgctgagtagtgtgggttatggtatgttccc |
| 1814 | NM_002985 | CCL5 | gcagtcgtctttgtcacccgaaagaaccgccaagtgtgtgccaacccaga |
| 1815 | NM_003002 | SDHD | aacgggcaatcgcctagaaacgatagagacagaacgcatgcgcgcaggcg |
| 1816 | NM_003005 | SELP | cacatgtcaattcatctgtgacgagggatattctttgtctggaccagaaa |
| 1817 | NM_003006 | SELPLG | tgcagtatcccaactgcaggtccagtgcaggcaataaatatgtgatggac |
| 1818 | NM_003009 | SEPW1 | caaccacggcagggaaaagcggagtacggaacgaccatcgagcacaacag |
| 1819 | NM_003025 | SH3GL1 | gctagcaatatcgtgtgttgcgcactcgtaaagtgtgttatttcaaccct |
| 1820 | NM_003038 | SLC1A4 | ttccagtctcctgtgaagccaaaagggaccaggaaccgtgcaaaggaaac |
| 1821 | NM_003039 | SLC2A5 | agttcccgccagggttctacacaccgtctgcaagtcagtcaagtggatca |
| 1822 | NM_003058 | SLC22A2 | gaatggggatcacaatggcctatgagatagtctgcctggtcaatgctgag |
| 1823 | NM_003072 | SMARCA4 | tctctcaacgctgtccaactgggcgtacgagtttgacaagtgggccccct |
| 1824 | NM_003073 | SMARCB1 | gatgcatcgctgcactcaccctccgtgctgattccgccttagttctccac |
| 1825 | NM_003100 | SNX2 | tgaatcaaagagtatgtctgctcccgtgatctttgatagatccagggaag |
| 1826 | NM_003113 | SP100 | cagacgtacacccgagtagaagggtttgtgcaggacatgcgtctcatctt |
| 1827 | NM_003118 | SPARC | caccagattggtgtgtcatttttagaccctttactaggcatttatagatg |
| 1828 | NM_003120 | SPI1 | ctacaaagtttggcccctcccaatggtaagagggacaccccccttccttg |
| 1829 | NM_003121 | SPIB | aatgccaatgacctagagacacgagaagtccatgtggaggcacacagcag |
| 1830 | NM_003123 | SPN | ctaggatcccacaccgtgacaggtggaaccataacaacgaactctccaga |
| 1831 | NM_003127 | SPTAN1 | agaaaatggtgcttcactaacccgcttccggtccagtcacaatcatcatg |
| 1832 | NM_003128 | SPTBN1 | agcatgtcacgatgttacaagaacgattccgggagtttgcccgagacacc |
| 1833 | NM_003133 | SRP9 | catgttgaacagcattttagcatggtaagttcccttagctatatgaattt |
| 1834 | NM_003159 | CDKL5 | gtaaaccaagctgcgctcctgacataccatgagaatgcggcactgacggg |
| 1835 | NM_003189 | TAL1 | tggccgtacttgtgatttcgatggtacgtgaccctctgctgaagacttgc |
| 1836 | NM_003200 | TCF3 | cagctggctggatgattgggactttaaaacgaccctctttcaggtggatt |
| 1837 | NM_003222 | TFAP2C | aagtggtatctgacacactctaaaccccgtgttcaaacgggggccttctg |
| 1838 | NM_003225 | TFF1 | cagagwzgtgtacagtggccccccgtgaaagacagaattgtggttttcct |
| 1839 | NM_003234 | TFRC | ggactgacatttagcgtagctaagtgaaaaggtcatagctgagattcctg |
| 1840 | NM_003262 | TLOC1 | tgaattctatccgtgaatcccttagaggtctgtgaattgtaggttaagaa |
| 1841 | NM_003288 | TPD52L2 | ctgcatcctgacccacgtgtttgtagatcctttcagctattggcgccctg |
| 1842 | NM_003302 | TRIP6 | ctttgattgatcactctccctgacatccacctgtatgactttgtcaccaa |
| 1843 | NM_003311 | PHLDA2 | tcttcacgcgcccaccgccgcaatcgatccccccttattaCcttcttacc |
| 1844 | NM_003315 | DNAJC7 | caaatgctaaactctactgtaatcggggtacggttaattccaagcttagg |
| 1845 | NM_003318 | TTK | gtactatatgacttacgggaaaacaccatttcagcagataattaatcaga |
| 1846 | NM_003352 | SUMO1 | gccgatctacagtcattcaccgggggtaggagagtcacacaagtacaacc |
| 1847 | NM_003361 | UMOD | tctgggaccagattccgaagtgggagtgtcatagatcaatcccgtgtcct |
| 1848 | NM_003383 | VLDLR | ttcagctttggatgtggttaccgagtatctgtaacccttgaatttctaga |
| 1849 | NM_003390 | WEE1 | gcatctatgccgatctatgccgatctaagttgagcatcagtatggtgacc |
| 1850 | NM_003395 | WNT9A | ccgccgtccctgtgttatagtacataagaacccttattctcctgtggttc |
| 1851 | NM_003403 | YY1 | agcactaggactctagcctgcatttaggaagacttgccattttgccaagt |
| 1852 | NM_003415 | ZNF268 | cctgtcacttccagcgaggcacacaaaactgaccgtagggatggccacca |
| 1853 | NM_003425 | ZNF45 | atggtttggtcgtaggatcagcagggctcgcacttcggtcagcaggaaaa |
| 1854 | NM_003427 | ZNF76 | agagtccgccacccaaacgaccccggatagcttacctttcggaggtgaag |
| 1855 | NM_003453 | ZNF198 | tacagagttgcttacaatgacggatatgatgagtgaagacgaggggaaaa |
| 1856 | NM_003467 | CXCR4 | ggagtgggttgatttcagcacctacagtgtacagtcttgtattaagttgt |
| 1857 | NM_003480 | MFAP5 | agaatatcttcacgaacttgaccctcctactccacattgcaacatttcca |
| 1858 | NM_003549 | HYAL3 | ggagggaaccttgggtaggtttgagcagggggtatagtgtctgctttgca |
| 1859 | NM_003582 | DYRK3 | atgaaggagaccagttggcctgcatgatggagcttctagggatgccacca |
| 1860 | NM_003627 | SLC43A1 | tcagtgctgtgttcgccttgcttcagcagccacttttcatggcgatggtg |
| 1861 | NM_003636 | KCNAB2 | gcagtttgggagccacaaaaagcgtagcggtgtgatttctagctcagcct |
| 1862 | NM_003645 | SLC27A2 | gaggaaattttgtaggaaatttgcatacccgtaaagggagacttttttaa |
| 1863 | NM_003692 | TMEFF1 | acagactttagtattctctatgtagtgccaagtaggcaaaagctcactca |
| 1864 | NM_003701 | TNFSF11 | actaatggtgtacgtcactaaaaccagcatcaaaatcccaagttctcata |
| 1865 | NM_003707 | RUVBL1 | tgccatgttaaatatccctatctgtcggacacctgtatacatataagttt |
| 1866 | NM_003739 | AIKR1C3 | atttcacacaagacttctcccgccgtattggttgcggaccttgcgcggcc |
| 1867 | NM_003746 | DNCL1 | gataaaacgccgaacggaacacgaactgcgacactgaaagaacccacccg |
| 1868 | NM_003753 | EIF3S7 | tgtccgttgtgagcacgatggcgtcatgactggagccaacggggaagtgt |
| 1869 | NM_003801 | GPAA1 | ttctggaatgtgctcttctggaagtgagatctgcctgtccgggctgggac |
| 1870 | NM_003805 | CRADD | tggtagatctaaataccataaatggtggctagctttataggaaaacccat |
| 1871 | NM_003821 | RIPK2 | ccatatgatatacctcaccgagcacgtatgatctctctaatagaaagtgg |
| 1872 | NM_003824 | FADD | gatttcctgtagtgaatcaggcaccggagtgcaggttcgggggtggaatc |
| 1873 | NM_003842 | TNFRSF10B | acattgtaagatccatctacacagtcgttgtccgacttcacttgatacta |
| 1874 | NM_003852 | TIF1 | gtaggttagatgctattaagaaggcacttaatagtacatcatgtaagatg |
| 1875 | NM_003874 | CD84 | tagactcatctagcaccaactaccattagcactatgttaggagctgcaag |
| 1876 | NM_003877 | SOCS2 | agaggactgaccatattaaagagttagggctactaacctcaaaaagtttc |
| 1877 | NM_003879 | CFLAR | gagcaagcccctaggaatctgcctgataatcgattgcattggcaatgaga |
| 1878 | NM_003884 | PCAF | ggtcataaccccctaaaatccatcatgcaaccttattaatctgtcttggg |
| 1879 | NM_003900 | SQSTM1 | cacatgtgaactttttctaggtggcaggacaaatcgcgcccatttagagg |
| 1880 | NM_003902 | FUBP1 | ttcgtggcactccacaacagatagactatgctcggcaactcatagaagaa |
| 1881 | NM_003914 | CCNA1 | tgagtgagcttcataaagcgtaccttgatataccccatcgacctcagcaa |
| 1882 | NM_003954 | MAP3K14 | cacgtagcattaaatcagctgtgaatcgtcagggggtgtctgctagcctc |
| 1883 | NM_003955 | SOCS3 | tgtcagcccagtaagtattggccagtcaggcgcctcgtggtcagagcaga |
| 1884 | NM_003974 | DOK2 | ctggccctacaggtttctgcggcgctttgggcgggacaaggtaacctttt |
| 1885 | NM_003998 | NFKB1 | cgcaaactcagctttaccgagtctctgaccagtggtgcctcactgctaac |
| 1886 | NM_004039 | ANXA2 | agacagacgagacacgacagaccggaacgcacgaccaccgaacaagcaac |
| 1887 | NM_004047 | ATP6V0B | ttgctgcgtgttgatttggaggcactgcagtccaggCcgagtCctcagtg |
| 1888 | NM_004064 | CDKN1B | agacggggttagcggagcaatgcgcaggaataaggaagcgacctgcaacc |
| 1889 | NM_004067 | CHN2 | cagaagtgacatcctattgctttcaccatgttttatctgttagaagcaaa |
| 1890 | NM_004077 | CS | acctctttttgaacagggaattgattcaagattggacatggtctcctctg |
| 1891 | NM_004083 | DDIT3 | atgatgtgaccctcaatcccacatacgcagggggaaggcttggagtagac |
| 1892 | NM_004088 | DNTT | gtaatgggtaaggttctaataggccatgtttatgactgttgcatagaatt |
| 1893 | NM_004093 | EFNB2 | cagcttgtttaacggcagtgtcattcccctttgcactgtaatgaggaaaa |
| 1894 | NM_004099 | STOM | cttgcctgtacttgtcagaactagaggaaatagccaagactaatgaaaaa |
| 1895 | NM_004104 | FASN | gacattccacggggtcctactggatgcgttcttcaacgagagcagtgctg |
| 1896 | NM_004111 | FEN1 | cagaggagtaagatggtgatgttcacctggcaatcagctgagttgagact |
| 1897 | NM_004119 | FLT3 | aaacctcaagtgctcgcagaagcatcggcaagtcaggcgtcctgtttctc |
| 1898 | NM_004177 | STX3A | gggcattttagcattgattattggactttccgttgggctgaattaagagt |
| 1899 | NM_004208 | PDCD8 | ggcgggtagagcaccatgatcacgctgttgtgagtggaagattggctgga |
| 1900 | NM_004221 | NK4 | caaaaccctcaagaactgacagcattacactgaacaccgacaccaagagc |
| 1901 | NM_004235 | KLF4 | caatggtttattcccaagtatgccttaagcagaacaaatgtgtttttcta |
| 1902 | NM_004310 | RHOH | ttgcttccctttccccacaagaacaaagcactggccgggtaaggtggctc |
| 1903 | NM_004313 | ARRB2 | tattcaccctgggcgcgcgtccactccccggatgttttctgtgcaatcga |
| 1904 | NM_004320 | ATP2A1 | aatcctcaagttcgttgctcggaactacctagagggataactgttccccc |
| 1905 | NM_004324 | BAX | tgccggaactgatcagaaccatcatgggctggacattggacttcctccgg |
| 1906 | NM_004335 | BST2 | ggtagtacttcttgtccgcgattctcacgcttaagacctggttttctctt |
| 1907 | NM_004336 | BUB1 | ttccagaatggcagtgtattagtaggagagctctacagctatggaacatt |
| 1908 | NM_004343 | CALR | gacgggtggacttcccgctggatcgaatccaaacacaagtcagattttgg |
| 1909 | NM_004355 | CD74 | cagtcatggatgaccagcgcgaccttatctccaacaatgagcaactgccc |
| 1910 | NM_004374 | COX6C | ttgttaggtgggtgtccacgagtgactagctgtctttccataggtctggt |
| 1911 | NM_004380 | CREBBP | caagcgctctaattcctataaatacgaaactcgagaagattcaatcactg |
| 1912 | NM_004385 | CSPG2 | gcaagataccgagacatgtgactatggctggcacaaattccaagggcagt |
| 1913 | NM_004413 | DPEP1 | tttcgggacgaggcagagaggatcatgagggactcccctgtcattgatgg |
| 1914 | NM_004425 | ECM1 | cttgtggtaaggttgggttcttgatgccggggggtgtcctttaaccccag |
| 1915 | NM_004430 | EGR3 | cctcgctccttctggtatatgcatgtcactgcatgataattgagttttcc |
| 1916 | NM_004448 | ERBB2 | tgttaggagcctcaaaaccttcttgtatcccttttacagtcaaagtccaa |
| 1917 | NM_004449 | ERG | catttggggactgtgtacaatgagttatggagactcgagggttcatgcag |
| 1918 | NM_004454 | ETV5 | taaggggtgcactttatagctatggaaacatgagattctcctctattgga |
| 1919 | NM_004475 | FLOT2 | aacaacgggaacgttactgactctggtgccttatctcgaagggaccagaa |
| 1920 | NM_004494 | HDGF | gtagctttggaagtttagctctgagaaccgtagatgatttcagttctagg |
| 1921 | NM_004501 | HNRPU | gggtaagctaaatcatagtttctgacaataactgggaaggttttttctta |
| 1922 | NM_004508 | IDI1 | aagcaatccagccgagcttgaggaaagtgacgcccttggagtgaggcgag |
| 1923 | NM_004513 | IL16 | agatgaacataatctaccaacgaaagacgtgattcaattcaacactccct |
| 1924 | NM_004526 | MCM2 | aactaccagcgtatccgaatccaggagagtccaggcaaagtggcggctgg |
| 1925 | NM_004530 | MMP2 | cccctgttcactctacttagcatgtccctaccgagtctcttctccactgg |
| 1926 | NM_004557 | NOTCH4 | atgtaagatgttctttcctatatatggtttccaaagggtgcccctatgat |
| 1927 | NM_004583 | RAB5C | gggcatactctgcgttaaggtggacgttttgaccgagtcgatgcgccgtt |
| 1928 | NM_004615 | TM4SF2 | tgatttaatgtagtactctgcttctgtatccccgaggtgagtcagaaaaa |
| 1929 | NM_004624 | VIPR1 | ccagaagatcccctcaggactgcaacaggcttgtgcaacaataaatgttg |
| 1930 | NM_004633 | IL1R2 | atttctaagtgtgagggggaccactcacttactcgtacacgatgtggccc |
| 1931 | NM_004642 | CDK2AP1 | cccaccaaacctaacaaggacccccgaacactttcatactaagactgtaa |
| 1932 | NM_004688 | NMI | tccaggactcatattggcgactggtaatcgggatataactaaaagataga |
| 1933 | NM_004689 | MTA1 | ttggaatatatggtccaatgaagcacatccctcgcgcccgacccgccctg |
| 1934 | NM_004706 | ARHGEF1 | ttttgcaagaaggagaggaatgggggagaggacgtgagggaccaccccca |
| 1935 | NM_004725 | BUB3 | aacaattgatcccagaagggcaaattgtttgagtcagtaatgagctgaga |
| 1936 | NM_004815 | PARG1 | tatttttgatccggattacatcaaggagttggtgaatgatatcaggaagt |
| 1937 | NM_004827 | ABCG2 | acctccttctgtcatcaactcagatgggtttccaagcgttcattcaaaaa |
| 1938 | NM_004867 | ITM2A | tggtgaaaatcgccttcaatacccctaccgccgtgcaaaaggaggaggcg |
| 1939 | NM_004924 | ACTN4 | aatgaagacatagccgattctctgcccgggccccttgctgatgctcctcc |
| 1940 | NM_004936 | CDKN2B | gctggaacctagatcgccgatgtagatttgtacaggagtctccgttggcc |
| 1941 | NM_004938 | DAPK1 | agcagcatcatgtgcttcgggtgtcacgacgtctactcacaggccagcct |
| 1942 | NM_004941 | DHX8 | gattgccaccaatatcgcagagacatcgctgactattgatggtatctact |
| 1943 | NM_004972 | JAK2 | caataatgtaaatcaacgcccctcctttagggatctagctcttcgagtgg |
| 1944 | NM_004994 | MMP9 | ttctgccaggaccgcttctactggcgcgtgagttcccggagtgagttgaa |
| 1945 | NM_004997 | MYBPH | aataccgcgccctctctgagcaaggcgtctgcaccctagagatccggaaa |
| 1946 | NM_005012 | ROR1 | gagtgtttagaaagtgtaagggctctgcaactaaaaggacctgacctaga |
| 1947 | NM_005080 | XBP1 | cagctgattagtgtctaaggaatgatccaatactgttgcccttttccttg |
| 1948 | NM_005084 | PLA2G7 | caacacatcatgttacagaactcttcaggaatagagaaatacaattagga |
| 1949 | NM_005085 | NUP214 | atccttcggcacgctcgcgagtcagaatgcccccactttcggatcactgt |
| 1950 | NM_005098 | MSC | tccaaaaagagcttcctttcgtgacgagacgcggacgcaggtccaccctc |
| 1951 | NM_005104 | BRD2 | atccacctccatccgcttggaaatggccttcgtcccggcctatgactggt |
| 1952 | NM_005123 | NR1H4 | gttccttcgttcagctgagattttcaataagaaacttccgtctgggcatt |
| 1953 | NM_005157 | ABL1 | ttcatccctctcatatcaacccgagtgtctcttcggaaaacccgccagcc |
| 1954 | NM_005161 | AGTRL1 | gaccgtgaggatgaaattaatagtggggcctttgtgagctagaggctggg |
| 1955 | NM_005178 | BCL3 | tgaacctgcctacacccctataccccatgatgtgccccatggaacacccc |
| 1956 | NM_005187 | CBFA2T3 | gcctgaagctgagcgtgtacgttaatgtgaatgtatatagtctttgcaga |
| 1957 | NM_005192 | CDKN3 | taccgaaaaaccttaatacactgctatggaggacttgcgagatcttgtct |
| 1958 | NM_005197 | CHES1 | acacttttgctatataacctaagtgataaccctcttttagttacctgcca |
| 1959 | NM_005215 | DCC | aattttccgggaactttgcagcataccaattacccataaacagcacacct |
| 1960 | NM_005225 | E2F1 | aaggaactgaggcctgggtgatttatttattgggaaagtgagggagggag |
| 1961 | NM_005230 | ELK3 | ctgtcagcatggaaagtcgggggctttcgcccgggtcctcctagaaattc |
| 1962 | NM_005239 | ETS2 | ttacatttaatagacttacagggataaggcctgtggggggtaatccctgc |
| 1963 | NM_005245 | FAT | ggatacatcagattggatgccaagcgttcctctgccggacatacaagagt |
| 1964 | NM_005246 | FER | tcattagacgctgctgctggtatgttgtatctcgagagtaaaaactgtat |
| 1965 | NM_005248 | FGR | tcccctacccaggcatgaataaacgggaagtgttggaacaggtggagcag |
| 1966 | NM_005252 | FOS | attgttgaggtggtctgaatgttctgacattaacagttttccatgaaaac |
| 1967 | NM_005263 | GFI1 | agactattccctccgtttagagaatgtaccggcgcctagccgagcagaca |
| 1968 | NM_005317 | GZMM | tccaagtgctggacacccgcatgtgtaacaacagccgcttctggaacggc |
| 1969 | NM_005318 | H1F0 | ctacaaggtgggtgagaacgctgactcgcagatcaagttgtccatcaagc |
| 1970 | NM_005334 | HCFC1 | taacccctgtgactcaatattaccatagtgcgatgtcgttttgtgctatt |
| 1971 | NM_005335 | HCLS1 | cttttgtgaggattcgttaggaagaaaagcgctaagttagtgaatgaaac |
| 1972 | NM_005339 | HIP2 | gtgtctgctggaagtctactgccattatagggaaccttgcttgttagctt |
| 1973 | NM_005340 | HINT1 | gttcaaagattagagcgagtacttaatggtaaaataccgtcttatatgca |
| 1974 | NM_005356 | LCK | catatgcaccttgtgtctgtacacgtgtcctgtagttgcgtggactctgc |
| 1975 | NM_005375 | MYB | ttggtcacaaattgactgttacaacaccatttcatagagaccagactgtg |
| 1976 | NM_005385 | NKTR | gtgtacttaatcaccttagtgccagtttaatccagttatgcagaagaaat |
| 1977 | NM_005424 | TIE1 | atgaagtgtacgagctgatgcgtcagtgctggcgggaccgtccctatgag |
| 1978 | NM_005427 | TP73 | cagtcaagccgggggaataatgaggtggtgggcggaacggattccagcat |
| 1979 | NM_005439 | MLF2 | tctcaatttaccacccaaacgcgccaccgacccgatcccatcgtgaccga |
| 1980 | NM_005494 | DNAJB6 | gccagtttcattctgacgagctctatagtagtccggtgtggacctctgcc |
| 1981 | NM_005504 | BCAT1 | taacaaggaataatcccacaatatacctagctacctaatacatggagctg |
| 1982 | NM_005521 | TLX1 | aagggcccccacatttgtgccgacactgttctccttcggtggaagagctc |
| 1983 | NM_005529 | HSPG2 | aggagtctctgccacccactcaggattgggaattgtctttagtgccggct |
| 1984 | NM_005533 | IFI35 | ttcgctctctccgctcctatacctctatctgtcgtgcttaaacctctgct |
| 1985 | NM_005556 | KRT7 | tgtctgcagtggagagagggggagggcacacatgtggaagcttcggaaag |
| 1986 | NM_005563 | STMN1 | cgcccgaagagcctcagtgcttacactaaggagtcgtcttctatgcaata |
| 1987 | NM_005566 | LDHA | cgtccctctttttgttggttaccccattatcgtttgccctagcccccgcc |
| 1988 | NM_005572 | LMNA | agtgcgtgaggagtttaaggagctgaaagcgcgcaataccaagaaggagg |
| 1989 | NM_005583 | LYL1 | cttgaccctaggaagtgcccggcgtaccagtaccttcgccatccttttgt |
| 1990 | NM_005589 | ALDH6A1 | caatgaaaccagatttctcacttgctcttcatacttctattttgaggtaa |
| 1991 | NM_005608 | PTPRCAP | aagaattccaagggagcggacagtggcacgctgtgattgacacccaggcg |
| 1992 | NM_005621 | S100A12 | aaaacccagccttgcccgtggggagtaagagttaataaacacactcacga |
| 1993 | NM_005651 | TDO2 | aatctttcaacatacctgattccccgacactggataccgaagatgaaccc |
| 1994 | NM_005654 | NR2F1 | agcccacccagcagaaatacaatccgagctacaaagcatgggaaaaagag |
| 1995 | NM_005671 | D8S2298E | caaagctcttggaggctttaaagttctttctgttgggtgtgcattacagt |
| 1996 | NM_005687 | FARSLB | tggaccctttttgtgaagattggtctctgtggtgtgattctcttcccagg |
| 1997 | NM_005730 | CTDSP2 | cagagccagcctagtcattatttgctgtcgggtttccagtttcaccgtgt |
| 1998 | NM_005731 | ARPC2 | gtttcttggtaaatccagaatcaggatacaatgtctctttgctatatgac |
| 1999 | NM_005738 | ARL4A | agctaagctggaagggtgcttatccctgcgtagaaacgcctgccaatgct |
| 2000 | NM_005761 | PLXNC1 | ttccggaattcagtactgtggcattaaacgtcgtctttgaaaaaatcccg |
| 2001 | NM_005767 | P2RY5 | acccgccgtttttgttcagtctacccactctcagggtaacaatgcctcag |
| 2002 | NM_005781 | TNK2 | ctacctcagaatgacgaccattacgtcatgcaggaacatcgcaaggtgcc |
| 2003 | NM_005794 | DHRS2 | ctgccagactagcaatttgggggcttactcatgctaggcttgaggaagaa |
| 2004 | NM_005808 | CTDSPL | tgggaatcgaacctagggatagtgctccacttctgacgatggagtgaaga |
| 2005 | NM_005809 | PRDX2 | aacttaccgatcatcgcacgccggagttcgatcacgagaccgacggggaa |
| 2006 | NM_005826 | HNRPR | tgaaactgtgtgacagctatgaaattcgccctggtaaacaccttggagtg |
| 2007 | NM_005835 | SLC17A2 | ttgtccaggaatcttctcagattgatcactgtgcgaaagctcttttcatc |
| 2008 | NM_005845 | ABCC4 | cacttccaatggacagccctcgaccttaactattttcgagacagcactgt |
| 2009 | NM_005870 | SAP18 | tgtacttaagcatcactcaagggtaagtgttctaggaaggttgatttagc |
| 2010 | NM_005895 | GOLGA3 | gtgtgtcacaacaggctagggcacttcacgatgtcactacttgtttttct |
| 2011 | NM_005900 | SMAD1 | ctgctattctgaaattgcctacatgtttcaataccagttatatggagtgc |
| 2012 | NM_005902 | SMAD3 | agacacatcggaagaggcgtgcggctctactacatcggaggggaggtctt |
| 2013 | NM_005907 | MAN1A1 | tcatttgatccgcaagtctagcagcggactaacttatatcgcagagtgga |
| 2014 | NM_005911 | MAT2A | agggagtgttccctatccaatcaatcttgcatgtaacgcaagttcccagt |
| 2015 | NM_005923 | MAP3K5 | gatgtttgccttagacagtatcattcggaaggcggtacagacagccatta |
| 2016 | NM_005933 | MLL | attgtcatctttgccatgcgtaagatctaccgaggagaggaactcactta |
| 2017 | NM_005936 | MLLT4 | cttgagcggcatcgaatagaggcagctatggaccgaaagtctgatagtga |
| 2018 | NM_005944 | CD200 | ggactgtgaccgactttaagcaaaccgtcaacaaaggctattggttttca |
| 2019 | NM_005954 | MT3 | tttttcaccggggcgtggctaccctgtcgtgacgcgtttccgcgtattgt |
| 2020 | NM_005956 | MTHFD1 | aaaggtgtccctacaggcttcattctgcccattcgcgacatccgcgccag |
| 2021 | NM_005957 | MTHFR | ttggagcccgtgcagtacaggcaaaacacgcaagggcatcaggcactggt |
| 2022 | NM_005965 | MYLK | cttatttgtgctgttatagaatatcagctctgaacgtgtggtaaagattt |
| 2023 | NM_006005 | WFS1 | ttcagccactcaggaccctggctttctgctccaaggcactgaacacagtc |
| 2024 | NM_006017 | PROM1 | gagattttcatctattatactttatcaaagattggccatgttccacttgg |
| 2025 | NM_006038 | SPATA2 | cgtaacttaccctatggacaaaagcaggaatgactaacacatcctaggag |
| 2026 | NM_006060 | ZNFN1A1 | tgtcgcttcctagaatccccttctccaaacgattagtctaaattttcaga |
| 2027 | NM_006079 | CITED2 | cccagcagagtgagctgttgactcgatcgaaaccccggcgaaagaaatca |
| 2028 | NM_006082 | K-ALPHA-1 | cttcaattaccctctacaccattattacacattcacacaac |
| 2029 | NM_006084 | ISGF3G | ataattctcagtagttgtccgtgataatcgtgtcctgaaaatcctcgcac |
| 2030 | NM_006098 | GNB2L1 | tgttgaaatgagagcggtttcttacagtctacccggcgttgtggcacatg |
| 2031 | NM_006101 | KNTC2 | actacaccataaaatgctatgagagttttatgagtggtgccgacagcttt |
| 2032 | NM_006113 | VAV3 | cctgttagagaccctcatgggctaggacttttcatctaggatagattcaa |
| 2033 | NM_006120 | HLA-DMA | gcagaccctaatctctcctcaccgacagatggcacttcacagaacagaaa |
| 2034 | NM_006142 | SFN | gtgacgacaagaagcgcatcattgactcagcccggtcagcctaccaggag |
| 2035 | NM_006152 | LRMP | gcaagaggactcatggacgtctctagaacatatcttgtggccatttacca |
| 2036 | NM_006184 | NUCB1 | ttgtggaaccagtgatgcctcaaagacagtgtcccctccacagctgggtg |
| 2037 | NM_006185 | NUMA1 | aagagccagacagcgccaactcatcgttctacagcacgcggtctgctcct |
| 2038 | NM_006187 | OAS3 | atggcgctggtacgtaaatagaccaatgcagttaggtggctctttccaag |
| 2039 | NM_006190 | ORC2L | ttgatcagcctggctggtaccgtctagtactatgcagcggtcctcaagtt |
| 2040 | NM_006191 | PA2G4 | cagagttgctgagttcagtattgctaatgcagtatttttcattgtgcctt |
| 2041 | NM_006196 | PCBP1 | tctcccagtctccgcaagggagagtcatgaccattccgtaccagcccatg |
| 2042 | NM_006209 | ENPP2 | ggcttgtgttgtaatagtgtgggccgctttgtctcaaatccgcagttcta |
| 2043 | NM_006216 | SERPINE2 | taacttgctggccttatgcttacatggtctcatgtaatgtgcacaacaca |
| 2044 | NM_006230 | POLD2 | acattttcagcgttggctgagttacatgtacagtgcttaggccacctggg |
| 2045 | NM_006231 | POLE | agacaaccaggtgatgcacttctaccgctggcttcggtcgccatcctctc |
| 2046 | NM_006235 | POU2AF1 | agcctaaaggtgatgggtcctcgcctaggcttagtgctaccatgtgggtt |
| 2047 | NM_006255 | PRKCH | cccaaagggaatagagattctccaggaatttcctctatgggaccttccca |
| 2048 | NM_006263 | PSME1 | ggctgcgataagtgaagagttgaaccacacaacccgcgttgtgaataatt |
| 2049 | NM_006268 | DPF2 | aaaggagcaacacactgcccctaggcgtgcgtgtggcccagtttctctct |
| 2050 | NM_006273 | CCL7 | tttgggttttcttgtccaggtgcttcataaagtcctggacccacttctgt |
| 2051 | NM_006286 | TFDP2 | gagattgctacacggagcagtccttgacgatcgtgtttctgacgtttctc |
| 2052 | NM_006299 | ZNF193 | tttcagtttcatattacccctatatgcatcaaagctgcaacttcccttgc |
| 2053 | NM_006301 | MAP3K12 | actgctgactggtgagatcccctacaaagacgtagattcctcagccatta |
| 2054 | NM_006302 | GCS1 | tgcctgtagcccatatgctagaggttggtgaccctgacgacttggctttc |
| 2055 | NM_006325 | RAN | gcattcttcttacttccccgtacgctttctgtgctattccgcctccccct |
| 2056 | NM_006329 | FBLN5 | tcctcatcttttcgcaccaccaagcctactaatgcccacgggctctcagg |
| 2057 | NM_00634-3 | MERTK | tgacgttcggaaccaagcagacgttatttacgtcaatacacagttgctgg |
| 2058 | NM_006354 | TADA3L | gctgaaggcacttagtgcccacaaccgcaccaagaagcacgacctgctga |
| 2059 | NM_006355 | TRIM38 | ctatgaggccggagttgtatccttttataacgggaatactggctgccaca |
| 2060 | NM_006362 | NXF1 | gaagcagtcccccctgtaaatagtccttggatattaccgtctggttgtcg |
| 2061 | NM_006427 | SIVA | ccccatgtcagtgccgtcatctagactgattatgcccgcagtgatcctgt |
| 2062 | NM_006472 | TXNIP | cctattgcactgtgttctcctactgcaaatattttcatatgggaggatgg |
| 2063 | NM_006495 | EVI2B | gataatacaatcatttccaccgcttgactcacttaacttgcccctgccac |
| 2064 | NM_006509 | RELB | tctgacccccatggcatcgagagcaaacggcggaagaaaaagccggccat |
| 2065 | NM_006522 | WNT6 | taaaggcctctggatactgggctccccagaactgctggccacaggatggt |
| 2066 | NM_006534 | NCOA3 | ctccaaggaatgcacccacgagccaacatcatgagaccccggacaaacac |
| 2067 | NM_006559 | KHDRBS1 | tgatacatacgcagaacaaagttacgaaggctacgaaggctattacagcc |
| 2068 | NM_006565 | CTCF | tttaagagagccatcagttagctatcagactctaggttgatgcattttgt |
| 2069 | NM_006579 | EBP | ctaagtgaccgccccacctgtggcttatacgtggcgtggcctctttcttc |
| 2070 | NM_006591 | POLD3 | cccaatatgctgcctcaactctgagctgtctgcaaggcttagtaagtatt |
| 2071 | NM_006602 | TCFL5 | caagccatcgtggttggctggaactagacgtgtgatttgaatgcaggagt |
| 2072 | NM_006676 | USP20 | ttctttgccgctgatgagttaaagggtgacaacatgtacagctgtgagcg |
| 2073 | NM_006739 | MCM5 | tgctggccaagcatgtcatcactctgcacgtgagcgcactgacacagaca |
| 2074 | NM_006747 | SIPA1 | cctctcagagaaggtctctcacttggagtccatgctcaggaagctgcagg |
| 2075 | NM_006748 | SLA | tggaactactttaactaatccatagggacttctggtatgctttcctctct |
| 2076 | NM_006759 | UGP2 | aaataaaacacgtgcagatgtaaagggcgggacactcactcaatatgaag |
| 2077 | NM_006762 | LAPTM5 | tgtcgctggccatccattcagtcgattcagtcataggcgaatctgttctg |
| 2078 | NM_006769 | LMO4 | ctgctaaaaggtcagagtaatgcagaatgcgtgccttcatctcagatttg |
| 2079 | NM_006803 | AP3M2 | tcaaagaccatgatggtatcggactagttttcagacactgcctgttgctg |
| 2080 | NM_006820 | IFI44L | ctagtgttcatggaggtagcattgaagatatggttgaaagatgcagccgt |
| 2081 | NM_006825 | CKAP4 | gtcggcaaggagcgtgcatactgcgtttgtgtaattgtttgctgtatctc |
| 2082 | NM_006845 | KIF2C | ctccaaaagaaagtcttcgaagccgctccactcgcatgtccactgtctca |
| 2083 | NM_006857 | RY1 | ttggtaaacattggcttgcctcccatgacttgccattataattaaaactg |
| 2084 | NM_006875 | PIM2 | ctaccaccacacaaacttagttcatatgctcttacttgggcaagggtgct |
| 2085 | NM_006904 | PRKDC | ggatcgactttgggcatgcgtttggatccgctacacagtttctgccagtc |
| 2086 | NM_006910 | RBBP6 | aggctagaacgtcagataaacatgattccactcgtgcttcctcaaataaa |
| 2087 | NM_006931 | SLC2A3 | aacttcatgtcaactttctggctcctcaaacagtaggttggcagtaaggc |
| 2088 | NM_006932 | SMTN | agagcgactgcacataggggaaaacacacttggggtcaggctttttgccc |
| 2089 | NM_006937 | SUMO2 | ttttttgtacgtagctgttacatgtagggcaatctgtctttaagtaggga |
| 2090 | NM_006938 | SNRPD1 | aactgtaaccattgaattgaagaacggaacacaggtccatggaacaatca |
| 2091 | NM_006963 | ZNF22 | tggggcatgactattcgatttgactcaagcttcagtagactcagaagaag |
| 2092 | NM_006981 | NR4A3 | ggattagtgggctgcgtttcaacattccgtgttcgtactcccttttgtat |
| 2093 | NM_006994 | BTN3A3 | acatggctcgtggagagaagtctttggcctatcatgaatggaaaatggcc |
| 2094 | NM_007022 | CYB561D2 | ttcaggctacaaccactagcacggctgacgatggccctttctgcggagac |
| 2095 | NM_007063 | TBC1D8 | ttcaaccaccgagtgatcggggcacaagttgaccagtctgtcttcgagga |
| 2096 | NM_007065 | CDC37 | ttctatttggcaaacagcaatgatcttccaataaaagatttcagatgctc |
| 2097 | NM_007079 | PTP4A3 | ttattagaccccggggcagtcaggtgctccggacacccgaaggcaataaa |
| 2098 | NM_007117 | TRH | ggtgtcccaacgtataaactatcacagtagcgctcatggctcgtaggttt |
| 2099 | NM_012092 | ICOS | ttgtaatttgaatctagtatggtgttctgttttcagctgacttggacaac |
| 2100 | NM_012120 | CD2AP | caggttttacgatgtataatttacctaatagaccaaactaactcatggag |
| 2101 | NM_012207 | HNRPH3 | caacatacaatcttacaacacatcttctactaactttcttaca |
| 2102 | NM_012223 | MYO1B | cgaccattgctgcatattggcatgggacccaggtacgtagagaatacagg |
| 2103 | NM_012267 | HSPBP1 | acacagttgccctaatgcggctggaatccccattgggggtaggtgagcag |
| 2104 | NM_012317 | LDOC1 | gtcttacatgctcgtgaacgagaaccgattctgcaacgacgccatgaagg |
| 2105 | NM_012323 | MAFF | cctccccactgtttatttattgcacggatctaagttattctccccagcca |
| 2106 | NM_012334 | MYO10 | tacagatataataaccatgttgactatgggggagagacgctgcattccag |
| 2107 | NM_012384 | GMEB2 | ccaaaataatggtgagtgcctaggcttcccgagttagctctttgggtggc |
| 2108 | NM_012417 | PITPNC1 | atatccaggagattcattaggtcatcagcgatcaaaggttagcattttat |
| 2109 | NM_013282 | UHRF1 | gggctgtacaaggtcaatgagtacgtcgatgctcgggacacgaacatggg |
| 2110 | NM_013314 | BLNK | taagaagctcatgtggacttgttctattgcctgacctgatgaactgttaa |
| 2111 | NM_013416 | NCF4 | atcctccttttgtcttcacctcgatgacgaaaacctaaggaggacagagg |
| 2112 | NM_014005 | PCDHA6 | cgccctaagtcctccagtctccttagagctagtacttactaagcatttac |
| 2113 | NM_014207 | CD5 | catctatagtttggccaccatacagtggcctcaaagcaaccatggcctac |
| 2114 | NM_014232 | VAMP2 | gaagttcagtatgttatgatggaccaataattctgcccttcgggtttctc |
| 2115 | NM_014244 | ADAMTS2 | accctctccgcccttggggtaatgtactttgtgatattgccacccgcccc |
| 2116 | NM_014298 | QPRT | ccacatggggttctgcggtgataatgagcacatagtgaggggtcagcaaa |
| 2117 | NM_014345 | ZNF318 | attgaactcattccctgttccacaaacccatatgtatcctttcctcaacc |
| 2118 | NM_014365 | HSPB8 | tccctccgcggattctccggggctggttcatcacctccgaatattcctgt |
| 2119 | NM_014575 | SCHIP1 | atgtgacctgctttcgatcataagactgtgttaagaccttctcctcagtg |
| 2120 | NM_014595 | NT5C | tttacggcagacgtacccccgttcaccccacgttactagtaggggaccga |
| 2121 | NM_014614 | PSME4 | acccagcgatgtgttgcagaaattatagctggtttaatcagaggttctaa |
| 2122 | NM_014624 | S100A6 | caccactacgcgcacgcagcgatataggcatcgcacctctatcatcgtca |
| 2123 | NM_014667 | VGLL4 | agtcctagatatacttactgctggtacagttgtactctaagattggtatt |
| 2124 | NM_014707 | HDAC9 | ttcctactgagtaaatcagcaacgaaagacactccaactaatggaaaaaa |
| 2125 | NM_014724 | ZNF305 | ctacctttatattctccaacacaagaccgtgtaggtgtcccttcagagag |
| 2126 | NM_014734 | KIAA0247 | aagtctttccatcatagcaggcgtgaccagggggtatctggggaccaggt |
| 2127 | NM_014735 | PHF16 | gaacaaaatcgtatccgagatacccactagagagcaagaataaccgtttg |
| 2128 | NM_014780 | CUL7 | gggtgaagacggccagaacttggagaagagacggaatcttctgaactgcc |
| 2129 | NM_014792 | KIAA0125 | aggagaaaacacggtaacgtaattagaatagtcagagaaaagtagccaga |
| 2130 | NM_014824 | FCHSD2 | gtatccttagaatccaaatgtctagataagttgaggacacatacctgcat |
| 2131 | NM_014890 | DOC1 | actgcaggataaccgaactcaaggcttaattaacggggcactaaacaaaa |
| 2132 | NM_014899 | RHOBTB3 | atattcactcccggaaatgtcgttgcttagtaatgtaacctggagctttt |
| 2133 | NM_014914 | CENTG2 | agctcggctcaaagtgcgaagttgtttagatttccacgttacacaaacac |
| 2134 | NM_015050 | KIAA0082 | gggtatatcagtgtgtcttgcagaatcttggatcattaaagataaacata |
| 2135 | NM_015069 | ZNF423 | acacgatgagccagcacgcacagtgagggatcgctcaacaggacacctct |
| 2136 | NM_015099 | CAMTA2 | acatgatctcactagccaagcagatcatcgaagccacaccggagcggatt |
| 2137 | NM_015147 | KIAA0582 | ccagttttagaggatgtccttgggaggatcgcaaagcagtctggtgagct |
| 2138 | NM_015149 | RGL1 | cagtttctggcaaccccgtgtatttccgttttccccctaaagaacatatc |
| 2139 | NM_015158 | ANKRD15 | acttgaatctcaaagaagtgcggtctatcggttgtggagattgttctgtt |
| 2140 | NM_015166 | MLC1 | acctggggaacgtgttcccggctgagatggattacttgcgctgtgctgca |
| 2141 | NM_015196 | KIAA0922 | aatactacccggggttcaacccgtttcgcgcctatatgaacctggacata |
| 2142 | NM_015210 | KIAA0802 | aggaacttcggcgagaactggaccgcgctaataaaaactgccgaatcctg |
| 2143 | NM_015261 | KIAA0056 | agttcagctgttgacgccttgcagaggctttgtagagcatctgcagagac |
| 2144 | NM_015335 | THRAP2 | gtgtactgccagtgcaggcgaccactgcctacacagatccacattaaatc |
| 2145 | NM_015436 | RCHY1 | ccgactttgacactgttatctccgtaatagctttaccattaggctaggat |
| 2146 | NM_015458 | MTMR9 | aagagtgctgtaaggtacaggtttccatattgtgaacctgtgtacgcaca |
| 2147 | NM_015474 | SAMHD1 | gacagtacttcagtccaaaatccaactcgcctccgagaagcatccaaaag |
| 2148 | NM_015528 | RNF167 | gttgtatccagcaccggaaacggctccagcggaatcgacttaccaaagag |
| 2149 | NM_015568 | PPP1R16B | atatcgtactgaggtaacttccacgtagccccttgccacgcggcaccggt |
| 2150 | NM_015570 | AUTS2 | acaggtcaaccttgtggagccatcgcgagttagagggtgaaagatggcag |
| 2151 | NM_015636 | EIF2B4 | ctggctaactggcagaaccacgcatccctacggttgttgaatctagtcta |
| 2152 | NM_015670 | SENP3 | tcacctattttgactcgcagcgtaccctaaaccgccgctgccctaagcat |
| 2153 | NM_015710 | GLTSCR2 | ttcacttaaccatacggtcagcctagttagagtatgtgattcagtggctt |
| 2154 | NM_015833 | ADARB1 | cacagtctatggaacgctaatggagtcagcccctaaagctgtttgctttt |
| 2155 | NM_015855 | WIT-1 | ggcccatgaaattgatgaactgagagttgcttccagtcctgagcgcacca |
| 2156 | NM_015869 | PPARG | ttgcttaccaaactggacatcattgatgatctggattcaggcagggtctg |
| 2157 | NM_015965 | GRIM19 | atcgcgctgttgccactgttacaggcagaaaccgaccggaggaccttgca |
| 2158 | NM_016041 | DERL2 | aattactggttggtttgtaaacaccatcctccattcgacccattgacttc |
| 2159 | NM_016091 | EIF3S6IP | ttcattccctggtagacaaatccaacatcaaccgacagttggaggtatac |
| 2160 | NM_016184 | CLECSF6 | ttggattggtatctgtcattgtagggatagataataagctcttcttattc |
| 2161 | NM_016195 | MPHOSPH1 | tcaaagcactcgatttccaaaacctgagttagagattcaatttacacctt |
| 2162 | NM_016221 | DCTN4 | ctgctgttaatgatccagcattggtcacaatgtaccaactgctttctgca |
| 2163 | NM_016308 | UMP-CMPK | caacttgtacaaagttgtatgacagggcatattctttgcttccaagattt |
| 2164 | NM_016343 | CENPF | gacaggaaagactagcccatatatcctgcgaagaacaaccatggcaactc |
| 2165 | NM_016570 | PTX1 | aataatttgctgtcgtttcagacttggatcctataaacctgtcaattctg |
| 2166 | NM_016734 | PAX5 | gcctggggtaattggaggatccaaaccaaaggtcgccacacccaaagtgg |
| 2167 | NM_017437 | CPSF2 | ggaaggatcgatcttatttaactatgtgtggaacaacccagtaatatcag |
| 2168 | NM_017448 | LDHC | gttgtaatctagactctgcccgtttccgttacctaattggagaaaagttg |
| 2169 | NM_017617 | NOTCH1 | tcactgtgggcgggtccaccagtttgaatggtcaatgcgagtggctgtcc |
| 2170 | NM_017771 | PXK | atggaactcttctttggggactattactttgcaaatggggtcagagcctc |
| 2171 | NM_017787 | C10orf26 | cattgatcttacgcatttactgtttaccgaacaaatgtctgactgtgtac |
| 2172 | NM_017794 | KIAA1797 | cctaagagaggagtgcattgctttagtacccgggccagttgagactgaaa |
| 2173 | NM_018136 | ASPM | tacagttatcctacaagcactggtgcgtggttggctagtacgaaaaagat |
| 2174 | NM_018209 | ARFGAP1 | tcagccacttgcagaacaggatgggaccgagatttcagcgagccctcctg |
| 2175 | NM_018248 | FLJ10858 | aaattaatatgacagatggccctcgtaccttaaatcctgacagccctcgc |
| 2176 | NM_018462 | C3orf10 | agagtacattgaagctcgggtgacaaaaggtgagacactcacctagaaca |
| 2177 | NM_018664 | SNFT | agcattgttcaaatgagtacactgtcagtggggtttaagatcatttggtg |
| 2178 | NM_018951 | HOXA10 | gtattgctgctgtgcgtggaaaaagacgatgtttatgttcttatagaata |
| 2179 | NM_019028 | ZDHHC13 | cactgccatgtatgcaactgctgtgtggctcgatatgatcaacactgcct |
| 2180 | NM_019071 | ING3 | tgccctatagaatggttccattatggctgcgttggattgacagaggcacc |
| 2181 | NM_019102 | HOXA5 | tcatagttccgtgagcgagcaattcagggactcggcgagcatgcactccg |
| 2182 | NM_019111 | HLA-DRA | aaagatcgtaacgcgagagccatccgccctatgtgggagatcccacaccc |
| 2183 | NM_019841 | TRPV5 | tgattaacatcgctatcactcttgaccttactcccggttggcctgggggc |
| 2184 | NM_019846 | CCL28 | atacggccataaaactccttattagagagtctacagataaatctacagag |
| 2185 | NM_019857 | CTPS2 | aactggccttaggagatccaaatagggaaccaagattgcagagtgtccca |
| 2186 | NM_020310 | MNT | tctagtgccgaatgactatgtccagattggtgacgatgggtgttgctgtt |
| 2187 | NM_020368 | SAS10 | gcaggtcccacgctcaccgacgaaaatggagatgatttaggattgccacc |
| 2188 | NM_020371 | AVEN | cgactcaacgttgctgccgaactggtccagggtacagttcctttagaggt |
| 2189 | NM_020631 | KIAA0720 | tcaccttcgaggcctacaggttcgggggacactaccttcgtgtcaaagcc |
| 2190 | NM_020657 | ZNF304 | tctagaggggacatccacaagtctcggtgcagttatgatggtgtgggatg |
| 2191 | NM_020841 | OSBPL8 | ggtgacccacctcttaggactttggtcttatccacgtgtatgttgttttc |
| 2192 | NM_020843 | ZNF291 | cctcacaaggtgatccttttaacaatcgagttcaggaccttatcagctac |
| 2193 | NM_020892 | DTX2 | acagggcctgatattagtgaaactacactgggatagcatcagccatttaa |
| 2194 | NM_020944 | GBA2 | tgcaggtttatcgggactattacctcacgggtgatcaaaacttcctgaag |
| 2195 | NM_020956 | PRX | cgaaagtgtcaaagattcggctgccggaaatgcaagtgccgaaggttccc |
| 2196 | NM_020998 | MST1 | tggacatgccaatacacattactgacaggtatgcccacctgacctgcacc |
| 2197 | NM_021019 | MYL6 | accagaacagagcacaacaaaggacgagcagcgccagccgagacgacgta |
| 2198 | NM_021025 | TLX3 | ggcgcctgtattatactttgtacttttgcccaaacgtgtaaataataaaa |
| 2199 | NM_021038 | MBNL1 | gtgtaaccatgattctattatgtattggtacgtctgtagaccaagatata |
| 2200 | NM_021071 | DO | gaatggcactctgtgctatgaggtgcattataggacgaaggatgtccact |
| 2201 | NM_021103 | TMSB10 | tccacatcacgactggggtctcgaagaggacgggggtaggaaatcctcca |
| 2202 | NM_021111 | RECK | tacagataattacctactctggctagaagctaggggtcccagtgaagagc |
| 2203 | NM_021114 | SPINK2 | actatactctcccttatcgcgcgtctatatggccccgatcacccttccgc |
| 2204 | NM_021603 | FXYD2 | ccccaggagcttactacaagtgctagaaatctggacaccaggccaaggaa |
| 2205 | NM_021643 | TRIB2 | cagtagagacaagcgggaaggataattagctgaaagctatgatgatataa |
| 2206 | NM_021814 | ELOVL5 | actgctaaaggacaagtttagtcatacactgcataaccatattttggtca |
| 2207 | NM_021906 | USP9X | aacagaacgtacaattcatgcataaccgaatgcagtacagtatggagtat |
| 2208 | NM_021949 | ATP2B3 | tctctgtcgtagacacaccgataggaccaatggccaatagatattaaaag |
| 2209 | NM_021960 | MCL1 | agcaggtggtaataccatgggtgctgtgacactaacagtcattgagaggt |
| 2210 | NM_022067 | C14orf133 | agaacaatgcccctgtgcagatattacaggagtatgtcaatctggtggaa |
| 2211 | NM_022127 | SLC28A3 | tgtggttttgccaatatcgggtccctaggaatcgtgatcggcggactcac |
| 2212 | NM_022161 | BIRC7 | cccttgcttggcgtgggggatggcttaactgtacctgtttggatgcttct |
| 2213 | NM_022334 | ITGB1BP1 | tttgaggtcacgcggctagtaattggagcctggtttagaaccaagtcagt |
| 2214 | NM_022366 | TFB2M | cgttccaaagattgtgcttataaatggctgtatgatgaaaccctggaaga |
| 2215 | NM_022436 | ABCG5 | agatgtccacacgaggggtcggagttacctgatcacatcgagagagtgct |
| 2216 | NM_022438 | MAL | ggatttattaaaaccaagttgtccctaaggaatcagatgtgggcatctct |
| 2217 | NM_022469 | GREM2 | aaacacgagcacactctcttcgaacccaattgtgggtgtagcaatgaaag |
| 2218 | NM_022549 | FEZ1 | cttgctcgctactttctagggtcttaatagtagtcaagaaaagggatctg |
| 2219 | NM_022716 | PRRX1 | ggctaattctaagcactaaagtctacatctaagctatagatttaagcttg |
| 2220 | NM_024006 | VKORC1 | tgctacgtgctcattagtgttcgtccgacgacgaccgtcatatgtccacc |
| 2221 | NM_024099 | MGC2477 | tacaaaaacaaatgagcccgggatagtggcacaggcatgtagttccaact |
| 2222 | NM_024319 | C1orf35 | aggatcagacggaaagcaggggagtttctcgggtcacccttgaagagagg |
| 2223 | NM_024408 | NOTCH2 | ttctttcacaccgtgtggtccgtgttactggtatacccagtatgttctca |
| 2224 | NM_024424 | WT1 | ttgtttcttcttttaccatccttggcacttttagcggggggagaggaggt |
| 2225 | NM_024713 | C15orf29 | cacgtctgtgatttgagaggtgagttatttaagaggccagttttcaggac |
| 2226 | NM_024728 | C7orf10 | actcttcctcataacctcatccaatacactcaaacaacact |
| 2227 | NM_025263 | PRR3 | agcatgatccccaccgctatggtctatctatgatcaccgtgctttgtgaa |
| 2228 | NM_030660 | ATXN3 | tgagatgtacagagagtttcttgctgggtcaataggatagtatcattttg |
| 2229 | NM_030674 | SLC38A1 | acacttgaatcttggtcgttggtatgtaatccactctctagagtccagtg |
| 2230 | NM_030915 | LBH | ttatttttgctggtccctagaccactttgtatgaccgtttgcagtctgag |
| 2231 | NM_030926 | ITM2C | catgtttttactgatcattcgatatgctaaccgttctcagccctgagcct |
| 2232 | NM_030935 | THG-1 | ggaaagctgtagacatgcggctgcggatggagttgggtgctccagaagag |
| 2233 | NM_031243 | HNRPA2B1 | ttcaattgatgggagagtagttgagccaaaacgtgctgtagcaagagagg |
| 2234 | NM_031423 | CDCA1 | aaagaaaagttgaagcgaatggaagtatcagaagtaccaaataatgttgg |
| 2235 | NM_031966 | CCNB1 | ttcctactgtagggtagcggaaaagttgtcttaaaaggtatggtggggat |
| 2236 | NM_032121 | DKFZp564K142 | gtagaaacattttaggccatgtataagttacactgattttagcactaagc |
| 2237 | NM_033238 | PML | ctgttttcagatgtgattgaggggtgttctgccctgcctccactgtcaca |
| 2238 | NM_033512 | TSPYL5 | tattgaaaatccaagacactatgccaatgcaaccgtgactactttgggag |
| 2239 | NM_033554 | HLA-DPA1 | cttagatcccccctcggagtagcggctcacgcaacacaaagtatagcaca |
| 2240 | NM_033642 | FGF13 | ggaaacctggcacgggccaacttttcccgattatcacgccaagaagttgc |
| 2241 | NM_057168 | WNT16 | atactcaggataagatccttgaatatggaacttagttacaggactcaata |
| 2242 | NM_058195 | CDKN2A | atagatacatttcccgtatacttctgcgactatatctctctgtatttcta |
| 2243 | NM_080284 | ABCA6 | cctgtatcacttggttgggggatcctaattcatctaaggtttggccttat |
| 2244 | NM_080700 | TREX2 | cggctttagttacgatgactacagttcccacgtgcgcaactctgcccttg |
| 2245 | NM_130439 | MXI1 | aaacctatagtgcacattttaactgccccctaaattacccttccctgggt |
| 2246 | NM_133259 | LRPPRC | gagctaggtatagtgaccgtgaactttctaacgcataatattctgtgata |
| 2247 | NM_133378 | TTN | acgctccacccaaagccgaactggatgcccgattacacggtgatctggtt |
| 2248 | NM_138714 | NFAT5 | tagtgtacttcttgtctatcctcagttaatttacctagacaaaaagtgtc |
| 2249 | NM_144578 | C14orf32 | attcttaccattaagttaacaatggacgaagagatgacgctttgcttttt |
| 2250 | NM_144628 | TBC1D20 | ccttggcgtcgttttttgggtctgacttgtctcgtcaactgctggtctct |
| 2251 | NM_145698 | ACBD5 | gttcctgctttgtttaataagaacctcatttaaacttgacagctatggta |
| 2252 | NM_145867 | LTC4S | aactttaatgatagagactaggatgcggaagcgagcgcccctccccgggg |
| 2253 | NM_147180 | PPP3R2 | aattttacatggaggaagtgagcacatgtcatgctagccaagaggacatt |
| 2254 | NM_152739 | HOXA9 | atccccgcacaaagcggctctaaacctcaggccacatcttttccaaggca |
| 2255 | NM_152788 | EB-1 | gcttgtgcaaaaatgcgggctaactgtcagaagtctacagagcaaatgaa |
| 2256 | NM_152827 | SNX3 | agaaactggtataacatgccttcagtatactaacactcatatgctcagtt |
| 2257 | NM_152890 | COL24A1 | acacaaacaagtggcccaggattgcctattggtttcaagggatggaatgg |
| 2258 | NM_156038 | CSF3R | gcctcccaggcgatctgcatactttaaggaccagatcatgctccatccag |
| 2259 | NM_172020 | POM121 | catatatagatttctagaactccctctttgcatagtgccctcctttcttt |
| 2260 | NM_173156 | C1orf16 | ctaaaatcagtgctatctacaagccgaaatttaagcaacaactgtgacac |
| 2261 | NM_173216 | SIAT1 | ggtagagaaggatacagtgtctatcctcaagttgctacggttcagtgaga |
| 2262 | NM_173609 | C15orf21 | cactttgaacgccttagaggcaaaaagagctttgtgagttgtaagaggaa |
| 2263 | NM_173852 | KRTCAP2 | agacagatgacgcttttcgattctgcaccttgtatagctcctggagttgg |
| 2264 | NM_175739 | SERPINA9 | gccttccgcctataccgcaggctggttttggagaccccgagtcagaacat |
| 2265 | NM_175744 | RHOC | tccaggtccgcaagaacaagcgtcggaggggctgtcccattctctgagat |
| 2266 | NM_178586 | PPP2R5C | aatgtcactattccaagtgtctttttagtgtggctttagtatggcttcct |
| 2267 | NM_181311 | TAZ | caccagggaaagtgaacatgagttccgaattcctgcgtttcaagtgggga |
| 2268 | NM_181336 | LEMD2 | tcgtcctgacacgattccaaaggtctgccatactcttcagaggagttcgg |
| 2269 | NM_181339 | IL24 | aggaggtgggatatcacttccatgacataagtgctattgcagagccgtgg |
| 2270 | NM_181430 | FOXK2 | caagttcagacgaagctactctggcatctgcacatttccgtgttacagca |
| 2271 | NM_181493 | ITPA | atccttccttcgtctggctcgtgattcacttattcgccctccctactttg |
| 2272 | NM_181702 | GEM | agatgtggtttcccccattgtagcagggagctagcgtattagccttgtgg |
| 2273 | NM_182729 | TXNRD1 | tgtgcagaggtattcacaacattgtctgtgaccaagcgctctggggcaag |
| 2274 | NM_182810 | ATF4 | ggtcccctagttgaggatagtcaggagcgtcaatgtgcttgtacatagag |
| 2275 | NM_183395 | CIAS1 | ggggttcagataatgcacgtgtttcgaatcccactgtgatatgccaggaa |
| 2276 | NM_198232 | RNASE1 | caattatattaaactcggccaaaccctctgcgggataaaggggttttgtt |
| 2277 | NM_198400 | NEDD4 | tcagtgcatttcacccaacgaaaagcagctacattagactgtgaaacaac |
| 2278 | NM_199335 | FYB | ggtctgtccagctttcacacattgatatgtgaggctacatttgcatgtta |
| 2279 | NM_199423 | WWP2 | gccggccagttaattcattctcagccaatgaaggtttgtctaagctgcct |
| 2280 | NM_201592 | GPM6A | taattttacaagttcttgtagtaggtagggggtactactagggatatctg |
| 2281 | NM_201632 | TCF7 | tctactccgccttcaatctgctcatgcattacccacccccctcgggagca |
| 2282 | NM_201998 | SF1 | cttttttcggtgttagattcaacactgcgctaaagcggggatgttccatt |
| 2283 | NM_212474 | FN1 | gtcaacgaaggcttgaaccaacctacggatgactcgtgctttgaccccta |
| 2284 | R14777 | CYFIP2 | agcagcgtcgctttgacctgttcgacttctgttaccacctgctaaaagtg |
| 2285 | R56397 | PIK3C3 | gtgagcagtaccaagagttccgtaaacagtgttacacggctttcctccac |
| 2286 | R59027 | GRP58 | ctctttttgtggatttgatgaccttatggatattgccaaagtttaaggca |
| 2287 | R72151 | GNL3 | ctgttctctgtataagttatggtatgcatgagctgtgtaaattttgtgaa |
| 2288 | R79128 | MAP3K1 | ctctgtatgtaacaatccatcattcaccttcactactggtagtaacatag |
| 2289 | R86893 | C6orf110 | cccgtgttacaacgtggctcgcctaatgttcctcgatgcagagaggaaga |
| 2290 | T07281 | NRIP1 | gactaataaaagtacaaggcacatatacgtgacagaattggtactcaatc |
| 2291 | T28925 | ITGAL | cgtacttagcagctatctctcagtgaactgtgagggtaaaggctatactt |
| 2292 | U60115 | FHL1 | atgtaatgacaatacatactaacattcttgtaggagtggttagagaagct |
| 2293 | U79271 | AKT3 | tctggtttagacttaatatgccacaacgcaccacgaccttcccagggtga |
| 2294 | W04885 | MYL4 | atcttcttgtcgtgaccccgatcacaaataacaaatgctcttaacactct |
| 2295 | X66087 | MYBL1 | ctggatatacatagtctgcaatcaccaaagtataatatcttgtaaggcta |
| 2296 | AK024272 | | gaggttgcgacaagctactccaatacccctgctgggatagtgtcacagaa |
| 2297 | AK025231 | IGLC2 | caaagctgaaaccaaatctttataacaagggtggttaggtcagcattctt |
| 2298 | AL080190 | | ccatgcttgaaaatgtctgaatggcagtaatgaggataagttttttaatc |
| 2299 | BU618233 | | actcctattattcatcgcaagactcttctatagtaatccagttcaagata |

TABLE 22
60mer polynucleotide probes, according to one embodiment

| | GenBank | | |
|---|---|---|---|
| SEQ | Accession No. | | |
| ID | for Target | | |
| NO: | gene | Symbol | 60mers |
| 2300 | AA594161 | MYH11 | aactgcggctggaagtcaacatgcaggcgctcaagggccagttcgaaagggatctccaag |
| 2301 | AA766908 | MME | acagagcagtaccagcgctctaaaagcacctccttgtcactttattactccagaacaac |
| 2302 | AB014540 | SWAP70 | ggctaagatagatacttgtgaatcaaagatagcacagaaatgaactaagtatatcccatt |
| 2303 | AF196185 | PARD3 | gtttcttaatcacgtgcggcggtgtctaagtggtgttaccagtgtacgcgcagtgacctt |
| 2304 | AI597616 | MRPL33 | taagctcattcttagtcgtagatagcttgggaatctcatatgcagagtatccatattgac |
| 2305 | AI634809 | ARID5B | ttctcttaccatccgatattaagttgggcatgatccaactagtatcactaccttttgaag |
| 2306 | AI672553 | AKAP12 | ctgcattcacacatggcatgaaataagtcaggttctttacaaatggtattttgatagata |
| 2307 | AI809213 | RGS13 | aggacagtatatgagatttctatcgtacagggcaagcctacattgtggacatattctaga |
| 2308 | AK022231 | STAT1 | tcgcactctgtgtatataacctcgacagtcttggcacctaacgtgctgtgcgtagctgct |
| 2309 | AK022293 | CTSD | gccagtaaaggcacccctagctcttgggatgtttgtatccaactccgggcaatgtggggc |
| 2310 | AK055652 | C3orf6 | ataatttagaaacgtgatctcttgagagagagactacgcattgcctgggcactttcagat |
| 2311 | AL080130 | FLJ14001 | gtgagaataaatttagctgcataaaacgttcggctcatttatctgacatcttagtcacat |
| 2312 | AL833316 | MIR | gccctcagtctttttgggccttaggcgtttcgttcatcctgctaaggggatgaagcaaac |
| 2313 | AW291384 | STS-1 | ggctgaagatacccgcttaaggatgctaacatttaccctggtactgccacattttctcta |
| 2314 | BG993697 | SMYD3 | tttcttgaaaacctcctgtagttcctgttgtgtacgggataaaggtgcttaccatggggc |
| 2315 | BI769730 | HLA-DRB1 | cactatcgccgaaatatgcacaatcgcggtgtacgaggagcatacgccacacttatgatt |
| 2316 | BQ632574 | RASA1 | cctccaactaaccagtggtatcacggaaaacttgacagaacgatagcagaagaacgcctc |
| 2317 | BX504817 | SFRS7 | tactggtctgcatcaagtttattcggtagtttgaccgctagtatgttggaagttatttgg |
| 2318 | H53164 | IRF8 | cagcctgagtcagcaccaatcccgcattcagaacctcccagtgaaagggcagccttcatt |
| 2319 | H57732 | TGFBR2 | tttttatatttttcgtcacgttccccctgtagcctggaaagggacggtaacaattaaaga |
| 2320 | L29376 | 3.8-1 | tgtttaaccttatatgtacagtttcacatatgtataaaaacagtagtttgggggcctctt |
| 2321 | M80899 | AHNAK | tgcaaagttgaaaggccctaaatttaagatgccagacatgcacttcaaggcccccaagat |
| 2322 | NM_000038 | APC | ttgccagctccgttcagagtgaaccatgcagtggaatggtaagtggcattataagcccca |
| 2323 | NM_000043 | FAS | accggggcttttcgtgagctcgtctctgatctcgcgcaagagtgacacacaggtgttcaa |
| 2324 | NM_000061 | BTK | tgggggtggaccgaatttggcaagaatgaaatggtgtcataaagatgggaggggagggtg |
| 2325 | NM_000075 | CDK4 | aaatatcagacgtcatattacacatagtatccgccccaaaaccgatgaacacacccagca |
| 2326 | NM_000109 | DMD | gaacatgcattcaacatcgccagatatcaattaggcatagagaaactactcgatcctgaa |
| 2327 | NM_000189 | HK2 | atttcaccaagcgtggactactcttccgaggccgcatctcagagcggctcaagacaaggg |
| 2328 | NM_000237 | LPL | agagagatgatcgtgcctataaatagtaggaccaatgttgtgattaacatcatcaggctt |
| 2329 | NM_000269 | NME1 | cgactaccaccgaacaccttccccgatgtgatcctcaactaaccgacaaaatatacacgg |
| 2330 | NM_000271 | NPC1 | tgactgaactgtgtctaagggtcggtcggtttaccactggacgggtgctgcatcggcaag |
| 2331 | NM_000295 | SERPINA1 | ggttgagtcataccaaatagtgatttcgatagttcaaaatggtgaaattagcaattctac |
| 2332 | NM_000313 | PROS1 | tcaaactgggttattcactgctgcacgtcagtcaactaatgcttatcctgacctaagaag |
| 2333 | NM_000397 | CYBB | attgacatctgagcatactccagtttactaatacagcagggtaactgggccagatgttct |
| 2334 | NM_000405 | GM2A | ccgctttaaggtccataaatatccctaaggaaaaatccaccgtggtacattcagtcctct |
| 2335 | NM_000424 | KRT5 | aagccatgttttatccttttctggagagtagtctagaccaagccaattgcagaaccacat |
| 2336 | NM_000483 | APOC2 | cctgcccgctgtagatgagaaactcagggacttgtacagcaaaagcacagcagccatgag |
| 2337 | NM_000576 | IL1B | gtagcagtgtctgtaaaagagcctagtttttaatagctatggaatcaattcaatttggac |
| 2338 | NM_000579 | CCR5 | aatgtctttccttttgctcttaagttgtggagagtgcaacagtagcataggaccctaccc |
| 2339 | NM_000584 | IL8 | gagttagaactattaaaacagccaaaactccacagtcaatattagtaatttcttgctggt |
| 2340 | NM_000585 | IL15 | agtgatgttgtcttatagtctctatattctattcagtctcggcttgtcttttctagtggt |
| 2341 | NM_000600 | IL6 | actggcagaaaacaacctgaaccttccaaagatggctgaaaaagatggatgcttccaatc |
| 2342 | NM_000626 | CD79B | atgaggacatagtgacgctgcggacaggggaagtgaagtggtctgtaggtgagcacccacg |
| 2343 | NM_000627 | LTBP1 | aatctgtcctggtggaatgggttatacggtttctggcgttcatagacgcaggccaatcca |
| 2344 | NM_000633 | BCL2 | gatattctgcaacactgtacacataaaaaatacggtaaggatactttacatggttaaggt |
| 2345 | NM_000655 | SELL | tatttgcactgtagcctcgccgtctgtgaattggaccatcctatttaactggcttagcc |
| 2346 | NM_000713 | BLVRB | cggataacagcgacaatcgaacgctgaccgcttagaccttcgtgagtagctcggtgtcgt |
| 2347 | NM_000785 | CYP27B1 | ccctaggaaggtgaatctgccctagcctggtttacggtttcttataactctcctttgctc |
| 2348 | NM_000788 | DCK | ctctcacttcgttggtgaccagtttcttaaactgaaagctttaatgttacatagtaaatg |
| 2349 | NM_000853 | GSTT1 | gaagcagaatccaggtatggctggtggggcaagtaaggcgactctacttggctaagcctt |
| 2350 | NM_000877 | IL1R1 | agaatcgattaaattcattaagcagaaacatggggctatccgctggtcaggggactttac |
| 2351 | NM_000917 | P4HA1 | cagacgaatgtcagcgtcacataaaaagagcatgtaggatgggacatattgggatgtatt |
| 2352 | NM_000927 | ABCB1 | agcggctccgatacatggttttccgatccatgctcagacaggatgtgagttggtttgatg |
| 2353 | NM_001067 | TOP2A | cttgcttcaaacggaatgacaagcgagaagtaaaggttgcccaattagctggatcagtgg |
| 2354 | NM_001068 | TOP2B | ccaacaccaactgcaaagtcgactcagagatgtagtctagccatgtgctcaggtacagac |
| 2355 | NM_001110 | ADAM10 | gtacactgtacttctgtgtgcaattgtaaacagaaattgcaatatggatgtttctttgta |
| 2356 | NM_001154 | ANXA5 | ttggtcatgagcatgctagtatgaataaggcaatgtgttaagcactggcatacaaatgca |
| 2357 | NM_001166 | BIRC2 | tggagttcatcagagttatggtgccgaattgtctttggtgcttttcacttgtgttttaaa |
| 2358 | NM_001196 | BID | tcttgctttcctcaaacaggatactgcctgctgtaagaccatcctctatggttgtacttt |
| 2359 | NM_001230 | CASP10 | cctggaactgtattttgaatccttaaaggtgagccctcatagggagatccaaagtcctgt |
| 2360 | NM_001238 | CCNE1 | tgaatttccttatggtatacttgctgcttcggccttgtatcatttctcgtcatctgaatt |
| 2361 | NM_001242 | TNFRSF7 | caggcaaggcacctgaattcccaccgcagagagtgttgtggccgtgaggacttttctgtc |
| 2362 | NM_001254 | CDC6 | cattgtggcattttagactcgttgagtttcttgggcactcccaagggcgttggggtcata |
| 2363 | NM_001350 | DAXX | agaatgttctgggataacatttggaggaaggtgggaagcagatgactgaggaagggatgg |
| 2364 | NM_001425 | EMP3 | gttatagatcttgttcccccccgcacctttcccgccgcgttttggcttcttccctctgtt |
| 2365 | NM_001552 | IGFBP4 | ggcatcttctggcttgactggatggaaggagacttaggaacctaccagttggccatgatg |
| 2366 | NM_001558 | IL10RA | cagaataatgactgacttgtctaattcgtagggatgtgaggttctgctgaggaaatgggt |
| 2367 | NM_001618 | PARP1 | tgggtagtacctgtactaaaccacctcagaaaggattttacagaaacgtgttaaaggttt |
| 2368 | NM_001621 | AHR | ccaagcggcatagagaccgacttaatacagagttggaccgtttggctagcctgctgcctt |
| 2369 | NM_001663 | ARF6 | gaacaataaccattggtgactggagcaggtaattatagcctgcagaaaaaattatctaag |
| 2370 | NM_001706 | BCL6 | tttgggaatgtttgtctcatcccattctgcgtcatgcttgtgttataactactccggaga |
| 2371 | NM_001760 | CCND3 | agggaggcaagccctgttgacacaggtctttcctaaggctgcaaggtttaggctggtggc |
| 2372 | NM_001762 | CCT6A | agaggtaaaaagatgatggaaggtgtggtgactaagggccacggttattgggtgaaattt |
| 2373 | NM_001770 | CD19 | cccaagattcacacctgactctgaaatctgaagacctcgagcagatgatgccaacctctg |
| 2374 | NM_001771 | CD22 | tctcctttggaagtgaggcattgcacggggagacgtacgtatcagcggccccttgactct |
| 2375 | NM_001779 | CD58 | gcattagttttggctgtcatcaacttattatatgactaggtgcttgctttttttgtcagt |
| 2376 | NM_001781 | CD69 | gcaatatgtgatgtggcaaatctctattaggaaatattctgtaatcttcagacctagaat |
| 2377 | NM_001782 | CD72 | aaggattggaagttgactgatgatacacaacgcactaggacttatgctcaaagctcaaaa |
| 2378 | NM_001826 | CKS1B | caaaccgagcgatcatgtcgcacaaacaaatttactattcggacaaatacgacgacgagg |
| 2379 | NM_001827 | CKS2 | atcgcgcgaccagctaaacactcctgcggtcgtatggtcgacaacaatcacacataaaat |
| 2380 | NM_001831 | CLU | gtcagtgtgattcacttaatgatatacattaattagaattatggggtcagaggtatttgc |
| 2381 | NM_001852 | COL9A2 | tgaaggaatagggcggctttccttccagcgagcatcattcggctgttaccaaaacaaaca |
| 2382 | NM_001853 | COL9A3 | ataaaaggttgtgtacaactccacgaggtgaaaaatattcagtaacttgtttacatagca |
| 2383 | NM_001877 | CR2 | cattactgtatatactttgccttccataatcactcagtgattgcaatttgcacaagtttt |
| 2384 | NM_001888 | CRYM | gagatcctcatttatgtttgtagttggaaagcaaagctaggtagccatttcttctgttct |
| 2385 | NM_001894 | CSNK1E | gctatccctccgaattctcaacatacctcaacttctgccgctccctgcggtttgacgaca |
| 2386 | NM_001951 | E2F5 | ttgacttctgacattccactttcctaggttataggaaagatctgtttatgtagtttgttt |
| 2387 | NM_001967 | EIF4A2 | ggactctttacctcgcggcggaagaatatagctgcggagttagttaaacctcgctttccc |
| 2388 | NM_002002 | FCER2 | ccagcggctttgtgtgcaacacgtgccctgaaaagtggatcaatttccaacggaagtgct |
| 2389 | NM_002051 | GATA3 | ttttaacatcgacggtcaaggcaaccacgtcccgccctactacggaaactcggtcagggc |
| 2390 | NM_002095 | GTF2E2 | tttcttcctcgtttgttactttagagcaagtttgcccatagtcttgaatgcaatatttgt |
| 2391 | NM_002101 | GYPC | catcttccagtaactgagcatacccagtgggccaacaagcacgctctaaatacaaaacca |
| 2392 | NM_002105 | H2AFX | ttggtcacgatccccgcctacccaccaatccttaacgcgcctcaccggaccttaacattc |
| 2393 | NM_002120 | HLA-DOB | cctgggtgaggtaaaggacattcatgaggtcaatgttctgggaataactctcttccctga |
| 2394 | NM_002122 | HLA-DQA1 | cctggggtaagccacccggctacctaattcctcagtaacctccatctaaaatctccaagg |
| 2395 | NM_002155 | HSPA6 | gctgtgactgtcagggctatgctatgggccttctagactgtcttctatgatcctgccctt |
| 2396 | NM_002189 | IL15RA | aggtgggagagatgctgagcccagcgaatcctctccattgaaggattcaggaagaagaaa |
| 2397 | NM_002211 | ITGB1 | ccatcttgtttcacactagtcacattcttgttttaagtgcctttagttttaacagttcac |
| 2398 | NM_002217 | ITIH3 | gtgtccacactgactacattgtccccaacctgttttgagtagacacaccagctcctgttg |
| 2399 | NM_002250 | KCNN4 | ggaagaagtgtaactcaccagcctctgctcttatctttgtaataaatgttaaagccagaa |
| 2400 | NM_002305 | LGALS1 | atcaactacatggcggcggatggagacttcaagattaagtgcgtggcctttgagtgaagc |
| 2401 | NM_002350 | LYN | tccaaaaatgcacccaactagctctatgtttacaaatggacataggactcaaagtttcag |
| 2402 | NM_002358 | MAD2L1 | catgttcgtgccagaacatgttacaccactcctacgatggcttgcccgattcgatgcccc |
| 2403 | NM_002426 | MMP12 | ttaagtttgaaaatagttaccttcaaaggccaagagaattctatttgaagcatgctctgt |
| 2404 | NM_002467 | MYC | gccataatgtaaactgcctcaaattggactttgggcataaaagaacttttttatgcttac |
| 2405 | NM_002526 | NT5E | tctgaacagtcactgtaaatcattcttaagcccagatatgagaacttctgctggaaagtg |
| 2406 | NM_002567 | PBP | agggcatttcaaatgacgagaagagctagtaatcacctgcacaaagggcgatggtggcgt |
| 2407 | NM_002646 | PIK3C2B | tatgcctcccgctatggcaaccgaaagaatgcgacgcctggcaagaaccgccggatttct |
| 2408 | NM_002661 | PLCG2 | gtgccctattcacactctgggaagacgctaatctgtgacatcttttcttcaagcctgcca |
| 2409 | NM_002692 | POLE2 | gcctttggatttccacccactgagccctctagtactactagggcatactatggaaatatt |
| 2410 | NM_002738 | PRKCB1 | agagagatccttaacagctagtgcccattagggggctaaacctaaagcctgggtggtgat |
| 2411 | NM_002752 | MAPK9 | atctctgtagacgaagctctgcgtcacccatacatcactgtttggtatgaccccgccgaa |
| 2412 | NM_002832 | PTPN7 | gcttccatggctcaaataataccctgggtatgcaggacccactataccttgcatttgctg |
| 2413 | NM_002838 | PTPRC | gacattcttccttatgattataaccgtgttgaactctctgagataaacggagatgcaggg |
| 2414 | NM_002891 | RASGRF1 | ggcacgcgcaaaggctacctgagcaagcggagttcggacaacacaaaatggcaaacc |
| | | | aag |
| 2415 | NM_002892 | ARID4A | aagttttatatttggtacattactatggatggaatgtcaggtatgatgagtgggtgaagg |
| 2416 | NM_002943 | RORA | ctctagggagacctaataaacacaactgtgttactaattgatgctatactgagctgacaa |
| 2417 | NM_002966 | S100A10 | ctcataccaactatcggcaagaacgcaccgtttccgcacttcatttttaccccgcgcttc |
| 2418 | NM_002967 | SAFB | aatgtgccgtgggttccacgccgtgtgcgcaagttccctgtgtgaaagcacgtctgtctt |
| 2419 | NM_003037 | SLAMF1 | caagctacggaacaggtgggcgcatgatgaactgcccaaagattctccggcagttgggaa |
| 2420 | NM_003088 | FSCN1 | ttgacatcgagtggcgtgaccggcgcatcacactgagggcgtccaatggcaagtttgtga |
| 2421 | NM_003177 | SYK | attgcagagtggcctagagcactctcaccccaagcggccttttccaaatgcccaaggatg |
| 2422 | NM_003217 | TEGT | tactaaaggcacttccttcctgttaaacccctgttaactctccataaatttggtgattct |
| 2423 | NM_003226 | TFF3 | ttcgcctgaccatccacttattctgccacccctgcatggtccccactgcacttaacaggg |
| 2424 | NM_003243 | TGFBR3 | ctgctttgtccaggtgagaacatccataatttggggccctgagttttacccagactcaag |
| 2425 | NM_003254 | TIMP1 | ggaaccctttatacatcttggtcatcttgatctcataacgctggtataaggtggtctggt |
| 2426 | NM_003290 | TPM4 | ggattaaggcccttatgagaagaggccagaacttggttatcctcttccaatgtgaggaca |
| 2427 | NM_003299 | TRA1 | cttgttttggatgccccctaatccccttctcccctgcactgtaaaatgtgggattatggg |
| 2428 | NM_003332 | TYROBP | gtgccatccctgagagaccagaccgctccccaatactctcctaaaataaacatgaagcac |
| 2429 | NM_003362 | UNG | gctgggagcagttcactgatgcagttgtgtcctggctaaatcagaactcgaatggccttg |
| 2430 | NM_003451 | ZNF177 | ctaaatgaaacaacaggggagatacaatattatctggttgcactaatcctgtgctgctta |
| 2431 | NM_003648 | DGKD | tgatgctggatcttgccaagcgcagtcgcagtggtaaattccgcctcgtgaccaagttta |
| 2432 | NM_003656 | CAMK1 | actcacagctgcatgatgaggtagaggtggcccccactctcatagatgtcatccagggct |
| 2433 | NM_003810 | TNFSF10 | tccttaagtcaaaagagagaagaggcaccactaaaagatcgcagtttgcctggtgcagtg |
| 2434 | NM_003875 | GMPS | ctgtggttattcgaacctttattactagtgacttcatgactggtatacctgcaacacctg |
| 2435 | NM_003959 | HIP1R | tttgcccgtcagatttgaacgaatgtgtgtcccttgagcccaaggagagcggcaggaggg |
| 2436 | NM_004049 | BCL2A1 | cctataaggagatttcatattttgttgcggagttcataatgaataacacaggagaatgga |
| 2437 | NM_004073 | PLK3 | cgaatgggttggcttctccaataagttcggctttgggtatcaactgtccagccgccgtgt |
| 2438 | NM_004091 | E2F2 | tttatgagcaccatgtaagcctccttgtattgagataattgggcattaaacattaaactg |
| 2439 | NM_004117 | FKBP5 | gtggttctgagcaaataactacagggtgcccattaccactcaagaagacacttcacgtat |
| 2440 | NM_004126 | GNG11 | cggcagcaatatcacccaagagccataatacacctattataagcgaccagcataataaag |
| 2441 | NM_004184 | WARS | cccctcccaaaaagaaattgataagtaaaagcttcgttatacatttgactaagaaatcac |
| 2442 | NM_004271 | LY86 | gaagcggcaatcagcccgcgcgtggaaccgagacaagtaaacaagacgccagatctct |
| | | | ga |
| 2443 | NM_004289 | NFE2L3 | tggcagccatcctttttaagagtaagttggttacttcaaaaagagcaaacactggggatc |
| 2444 | NM_004322 | BAD | gaggccctgtgcctagcacaaaataagggcataagaaatactagttacatcacaaggtca |
| 2445 | NM_004354 | CCNG2 | ggttgttaaacagtaatagataagtgagacagattgcttgttatttatggtcaaatggtg |
| 2446 | NM_004356 | CD81 | ctttctaacacgtcgccttcaactgtaatcacaacatcctgactccgtcatttaataaag |
| 2447 | NM_004358 | CDC25B | cccactcggtcccagttttgttgccccagaaagggatgttattatccttgggggctccca |
| 2448 | NM_004422 | DVL2 | tcagcctaccagctgctgccatacaggagattgtggccactgtgactctcaccagcagtg |
| 2449 | NM_004445 | EPHB6 | tgcccgggaagtcgatcctgcttatatcaagattgaggaggtcattgggacaggctcttt |
| 2450 | NM_004510 | SP110 | tcccatctttagaagcaatctaccatagttcaccctctggccacagtagttcacatcttt |
| 2451 | NM_004529 | MLLT3 | tattccatcttctccataagtggagaaatgaaaagtaagaacagctcatcttcaaagttt |
| 2452 | NM_004556 | NFKBIE | cttgttttacccatgttgggtcagcctgaaactgccaaccagtaggaagcatggactctc |
| 2453 | NM_004577 | PSPH | agctagccaaaatctgtggcgttgaggacgcggtgtcagaaatgacacggcgagccatgg |
| 2454 | NM_004619 | TRAF5 | gagcaagactgtccttttaagcactatggctgtgctgtaacggataaacggaggaacctg |
| 2455 | NM_004635 | MAPKAPK3 | cttgctgcatttgatcctctacaactggtgttgctcccgttatgcggacagaagacagcc |
| 2456 | NM_004737 | LARGE | tggagaaatgcgagacaatccacgttgctattgtctgcgctggatacaatgccagccggg |
| 2457 | NM_004844 | SH3BP5 | ctttgctatcattctagggctggatttccctaactccagttattctgctgctgccgccat |
| 2458 | NM_004951 | EBI2 | aatgggctttgactggagaatcggagatgccttgtgtaggataactgcgctagtgtttta |
| 2459 | NM_005013 | NUCB2 | gctaatcaaagcggcaacaagtgatctggaacactatgacaagactcgtcatgaagaatt |
| 2460 | NM_005077 | TLE1 | cagaggggccatgggttgtaggattgaggaacggaatctgccgactcacatgacagccca |
| 2461 | NM_005127 | CLECSF2 | tgctatttatactttgagttactcttcaaaagtggtatacctctagtttggagctgtgct |
| 2462 | NM_005211 | CSF1R | actcccccaagctgactcatcctaactaacagtcacgccgtgggatgtctctgtccacat |
| 2463 | NM_005213 | CSTA | gcgggtttggcctcagataagcctccaggtatcattttggctgattgcttctttgctgga |
| 2464 | NM_005214 | CTLA4 | agccaggtgactgaagtctgtgcggcaacctacatgatggggaatgagttgaccttccta |
| 2465 | NM_005271 | GLUD1 | gggccttttgaacagtagtgtccccatgaagtgctagataatatatgtgtaagagtcagc |
| 2466 | NM_005292 | GPR18 | tcaggctcgagtcattagtgtcatgctataccgtaattaccttcgaagcatgcgcagaaa |
| 2467 | NM_005347 | HSPA5 | ccttacctcaaaggctctcactaccgtggaccacctagtctgtaactctttctgaggagc |
| 2468 | NM_005348 | HSPCA | ccctgtagttgacaattctgcatgtactagtcctctagaaataggttaaactgaagcaac |
| 2469 | NM_005415 | SLC20A1 | ttcgggcaagttaaatgggacagccttccatgttcatttgtctacctcttaactgaataa |
| 2470 | NM_005449 | TOSO | cagccccctgtagttgacaggtaagctgtaggcatgtagagcaattgtcccaatgccact |
| 2471 | NM_005461 | MAFB | gtgcactggtaagcagacactgcactgaaccaactgctatgctcagaatgtaccagaaac |
| 2472 | NM_005531 | IFI16 | tgaaactcacctgctttgaattggcaccgaaaagtgggaataccggggagttgagatctg |
| 2473 | NM_005582 | LY64 | gcgacagcattgattctcttagccatcttaagggaatctacctcaatctggctgccaaca |
| 2474 | NM_005601 | NKG7 | ctcgtccactcccccgctatccaacgtcacatctaagcactatgacatgcaccgaccgaa |
| 2475 | NM_005606 | LGMN | actgcttcaactggcactcccccacgtacgagtatgcgttgagacatttgtacgtgctgg |
| 2476 | NM_005610 | RBBP4 | ccattgctctgcatctctagcccactttatatcctgccaccagtacttcttgtaatccca |
| 2477 | NM_005623 | CCL8 | ctgtctgtagtgttgtggggtcctcccatggatcatcaaggtgaaacactttggtattct |
| 2478 | NM_005638 | SYBL1 | ctgaataaaattgatgtgcatgttttagggatcaattacctaactgttccttggtctatt |
| 2479 | NM_005652 | TERF2 | tatgtaggcaggttaatcctccacttctcatgtggttgaaccagtgtgttttttggtaaa |
| 2480 | NM_005658 | TRAF1 | taacctcttgatccctactcaccgagtgttgagcccaaggggggatttgtagaacaagcc |
| 2481 | NM_005856 | RAMP3 | agacgaggttctcatcccgctgatcgttatacccgtcgttctgactgtcgccatggctgg |
| 2482 | NM_005935 | MLLT2 | tgtagtcatatctgagcataagaccttgatgtgtgattcctgatgaccggtttcatttat |
| 2483 | NM_005950 | MT1G | aatacaaagatctcggattatccatcacctcttgtacaattacaccccaagtccctcgtc |
| 2484 | NM_006006 | ZBTB16 | ttgcggctgagaatgcacttactggctcattcagcgggtgccaaagcctttgtctgtgat |
| 2485 | NM_006014 | DXS9879E | tacaacacaacaccagcgcccaatatacgcgaatgaggcgccctatacccgcctctccac |
| 2486 | NM_006115 | PRAME | tgtcctgagtctaagtggggtcatgctgaccgatgtaagtcccgagcccctccaagctct |
| 2487 | NM_006162 | NFATC1 | gtacaattagttgcttattacgtatgattactcacagcgatctattgttccatataacca |
| 2488 | NM_006164 | NFE2L2 | atcctcatttataaacctggggtttgaactgcggtctccacgatcactgtcagctctcct |
| 2489 | NM_006195 | PBX3 | acagctttattacctcatgcgaactcatacaaaccaatagaatttcaacatgttctgtag |
| 2490 | NM_006218 | PIK3CA | ggggatgatttacggcaagatatgctaacacttcaaattattcgtattatggaaaatatc |
| 2491 | NM_006254 | PRKCD | gccttcggagggaaattgtaaatcctgtgtttcattacttgaatgtagttatctattgaa |
| 2492 | NM_006257 | PRKCQ | ctcagccgcagacaagtaatcactaacccgttttattctattcctatctgtggatgtgta |
| 2493 | NM_006317 | BASP1 | ttagcctacctagatttctcatgacgagttaatgcatgtccgtggttgggtgcacctgta |
| 2494 | NM_006332 | IFI30 | aagaagtccaatgcaccgcttgtcaatgtgaccctctactatgaagcactgtgcggtggc |
| 2495 | NM_006475 | POSTN | atactggaaaccatcggaggcaaacagctcagagtcttcgtatatcgtacagctgtctgc |
| 2496 | NM_006498 | LGALS2 | gccacgagggaactggcttacgctgttggcacatttccttccagacggttctccacagat |
| 2497 | NM_006558 | KHDRBS3 | aagcttccatcagccaccgaatgcttaagaactcatctaagccagtcctctcattgattg |
| 2498 | NM_006620 | HBS1L | acactaccaaactgtcagtgaacccgccgttattaaacgattgattagtgtcttaaacaa |
| 2499 | NM_006644 | HSPH1 | gtcatgaatgctcaggctaaaaagagtcttgatcaggatccagttgtacgtgctcaggaa |
| 2500 | NM_006734 | HIVEP2 | tcgatgagaatatgacccaaaggacactggtcaccaacgcagccatgcaagggataggat |
| 2501 | NM_006763 | BTG2 | aaacaggtccctgcctttttagaagcagcctcatggtctcatgcttaatcttgtctctct |
| 2502 | NM_006841 | SLC38A3 | ttcagctggctgcggcatgtgcttattgccgttggcctgctcacttgtatcaacctgctg |
| 2503 | NM_006889 | CD86 | actcctagcacctggctagtttctaacatgttttgtgcagcacagtttttaataaatgct |
| 2504 | NM_006892 | DNMT3B | ggaaccgtgaagcacgaggggaatatcaaatacgtgaacgacgtgaggaacatcacaa |
| | | | ag |
| 2505 | NM_006986 | MAGED1 | ttatgttcacagatattgctatcaatcgcagtagtctttcccctgtgtgaggctgaagcc |
| 2506 | NM_007311 | BZRP | tgtctagcgggctcctcacactcaacatgtcctaaatgctgggtatcgcatgaagcactg |
| 2507 | NM_007360 | KLRK1 | acaggttcacagttaagaaggaattttgcctctgaataaatagaatcttgagtctcatgc |
| 2508 | NM_007361 | NID2 | ggccagtttactgatgagtatctcccagaacaacgatctcacctctacgggataactgca |
| 2509 | NM_012203 | GRHPR | tatatgtacttgtcacattggtgttggacacatttgcgccaaaagtatggtaattctatt |
| 2510 | NM_012448 | STAT5B | tccagcaaccctggttggggggtcagacttgatacactttcaggttgggagtggacccac |
| 2511 | NM_012452 | TNFRSF13B | catcagcccatctccagcgctgtcccaattacagccctagcaagtggagacaacaagaag |
| 2512 | NM_013230 | CD24 | aaaaatagacactccccgaagtcttttgttcgcatggtcacacactgatgcttagatgtt |
| 2513 | NM_014246 | CELSR1 | ataaaaaactctagtccctttaaacgtttgcccctggcgtttcctaagtacgaaaaggtt |
| 2514 | NM_014257 | CD209L | gaaatgagaacacggggtaactgcgatcattgcacttggtgttcacagtgggccaggcac |
| 2515 | NM_014338 | PISD | tgcaggagagacatcattgaattttttcaactgtatcagtagcacagtatttttgtatga |
| 2516 | NM_014456 | PDCD4 | catgtttattatctaatcattccaagttttgcattgatgtctgactgccactcctttctt |
| 2517 | NM_014686 | KIAA0355 | tgtgagactgtgtgaagccgtttgtgtggtctccatgtaggtgctgtgttcccggcaccg |
| 2518 | NM_014762 | DHCR24 | gatgaatggttaacacgagctagttaaacagtgccattgttttgccagtgaagcctccaa |
| 2519 | NM_015187 | KIAA0746 | agtggtttcgactggatatctcttttaacggaggccagatagtagtaaccactagcattg |
| 2520 | NM_015641 | TES | agccacccatttgcttttaatccaaagaacatgtatagtttttgtacccagagactatga |
| 2521 | NM_015866 | PRDM2 | taatgatgcgcttttgtcccgtaaatgttaacactcatgaagcataccccggcctctcag |
| 2522 | NM_016076 | PNAS-4 | agggaattctggttttagtccattggtagcattgtgtgtatgaggagattcaacaccaca |
| 2523 | NM_016081 | KIAA0992 | cagtcgctatgcagcactttcggaccagggactagacatcaaagcagcgttccaacctga |
| 2524 | NM_016187 | BIN2 | atagacacagacacttgcctatacagagacataaccacacacactcagaggatagtgaac |
| 2525 | NM_016336 | UBE2J1 | tcatcaacctacccaacctgtagctaagaatacctccatgagccctcgacagcgccgggc |
| 2526 | NM_016628 | WAC | aactggtatatgtcaggctttcccaatagcttaactgtgaccctatacggatggcttttt |
| 2527 | NM_017530 | LOC55565 | gtcattgggtcgccacctcttaatccactcggaggaccagcgatctaactgtgctgtgtg |
| 2528 | NM_017784 | OSBPL10 | ctactgtgagtgcgaggagaagagactgtgcgtcaacactcatgtatggaccaaaagcaa |
| 2529 | NM_017935 | BANK1 | ttctgaatttctcacattcagagttccagtcattattgttacatcatgtttgcagaaacc |
| 2530 | NM_017955 | CDCA4 | atctgaatccatgtcattcataaccccttgatctgtagtgtcatgggtgctgccgcaggc |
| 2531 | NM_018442 | IQWD1 | tattttaattaactgtcgacgtgctgccacgtctgttgctatttgcccaccaataccata |
| 2532 | NM_018842 | BAIAP2L1 | tcatgcttctctgtttcacgtagttgggttgacaagtttctgcctttaagataaatgagt |
| 2533 | NM_020151 | STARD7 | atgcacaagtgcgaaggagttgcggttgctccatgttctgacttagggcaatttgattct |
| 2534 | NM_020199 | C5orf15 | ggtcttggcgctcctgcttgtgtccgccgctctatccagtgttgtatcacggactgattc |
| 2535 | NM_020529 | NFKBIA | atgaacggtccatgccgataagtaaacgtcgaacaattgcccttcaaccactccctctaa |
| 2536 | NM_020992 | PDLIM1 | gtcccagagccaggttgtgaaggttttccttcactgcggagcccttaggttatgaacatg |
| 2537 | NM_021813 | BACH2 | agaaaagtctcattgtggcagattgagcataaattattcaactgacggtgcaaaaacatt |
| 2538 | NM_021822 | APOBEC3G | agtttcaaacctactaatccagcgacaatttgaatcggttttgtaggtagaggaataaaa |
| 2539 | NM_021950 | MS4A1 | cttagagttaccacaccccatgagggaagctctaaatagccaacacccatctgttttttg |
| 2540 | NM_021966 | TCL1A | tgcctgtagcccactctgctcgccttagcactaccactcctgcccaggaggattccattt |
| 2541 | NM_022552 | DNMT3A | aaccacagaggcggaaataccggagggctttgccttgcgaaaagggttggacatcatctc |
| 2542 | NM_023037 | 13CDNA73 | tgcttgagcaccagacaagcttggtatcttctgaggacggtgcccgagagcaggagaaca |
| 2543 | NM_024708 | ASB7 | aggtaccacaccgcttcagctcgccattatccgagagaggtcaagctgtgtgaaaatcct |
| 2544 | NM_025113 | C13orf18 | tgtggaactccagtagagcctagtaagtacggataatggatcgcctgcttatgatgtgat |
| 2545 | NM_025216 | WNT10A | tgtgagtaagcgagacacaacacttgtcctcttggaggttacattcttgctggggggagg |
| 2546 | NM_030666 | SERPINB1 | agagttacactctcaactccgacctcgcccgcctaggtgtgcaggatctctttaacagta |
| 2547 | NM_030764 | SPAP1 | tggtgtggaaaattacctggtgttgtagcgaagtagccatttccattctaacccagtcct |
| 2548 | NM_030775 | WNT5B | aagtccaaggtcatctctggcccagtgaccacagagagatctgcacctcccggacttcag |
| 2549 | NM_031305 | ARHGAP24 | ttcatatcttttcacctatttccagtccttatcatagttgataaaaacctcagactcatc |
| 2550 | NM_031942 | CDCA7 | taggtaattattgcagattgatgtctctcaatcccatgtattgcgcttatgttacaagtt |
| 2551 | NM_032991 | CASP3 | acgggagatttcttgttgctcaaaaaatgagctcgcatttgtcaatgacagtttcttttt |
| 2552 | NM_033208 | TIGD7 | catattgtccgttttccggtattgagttagggaaagtatgcaggtattcctgtcattcct |
| 2553 | NM_033274 | ADAM19 | agatcgccaactatgttgataagttttaccgatccttgaacatccggattgctctcgtgg |
| 2554 | NM_057735 | CCNE2 | gctttcttgacatgtaggttgcttggtaataacctttttgtatatcacaatttgggtgaa |
| 2555 | NM_078467 | CDKN1A | accctgtactgttctgtgtctttcacagctcctcccacaatgctgaatatacagcaggtg |
| 2556 | NM_079421 | CDKN2D | gttggaagcttgttttccagtctcttgtacagcgttttaaaagaaatggattctatttat |
| 2557 | NM_080593 | HIST1H2BK | gtgtgggccgtaaaaggaagggcaaaagctacactttagaatgcacgaggtccatctttc |
| 2558 | NM_138340 | ABHD3 | caattgatgattattatactgatgccagtccgagtcctagactgaagtcagtaggaattc |
| 2559 | NM_138379 | TIMD4 | cccaaaaaagaggccggagtgagagggagtgactattaacggcgggcgaaacgaaaa |
| | | | taa |
| 2560 | NM_139207 | NAP1L1 | tcctttctgagaagttagtgttaaggtcttggaatgtgaacacattgtttgtagtgctat |
| 2561 | NM_139276 | STAT3 | taatccctgaaacgggcttcaggtcaaacccttaagacatctgaagctgcaacctggcct |
| 2562 | NM_144646 | IGJ | atgcctgctatcctgactaatttaagtcattgctgactgcatagctctttttcttgagag |
| 2563 | NM_145804 | ABTB2 | ctagagctgtttgttcaatcactgatgggcaaaggggagatgtagctaactggtggccct |
| 2564 | NM_152785 | GCET2 | tagtactcttcagcagagttccctgatggccttgtacagagaacccgatgattgatgagg |
| 2565 | NM_172373 | ELF1 | gggctaatctggctgagtagtcagttataaaagcataattgctttatattttggatcatt |
| 2566 | NM_173624 | FLJ40504 | cagctttaacaaaaaattggccgtcaaaacctgagtcctgtcctcttgccctcctccccg |
| 2567 | NM_175870 | LOC90925 | aaaggaactcctgtcataaacattggatttcccgcggagtaacctggcgacatatttaga |
| 2568 | NM_177968 | PPM1B | gcagcgtgatgatacaacgtgttaatggttcattagcagtatctcgtgctctgggggact |
| 2569 | NM_181443 | BTBD3 | atagctgctgtgcaaattggtagaccttataagaaggcacttgtttgtaagccagagaag |
| 2570 | NM_181838 | UBE2D2 | aagagcacagtcttgtttggagagtctacataattactttgcactaacatttgcaggatg |
| 2571 | NM_182746 | MCM4 | cagagtgaatgttacaggcatctatcgagctgtgcctattcgagtcaatccaagagtgag |
| 2572 | NM_182776 | MCM7 | tctggcaggtcaatgcttcccggacacggatcacttttgtctgattccagcctgcttgca |
| 2573 | NM_198833 | SERPINB8 | aaccatacatcatctatgctttgtagtatgactcctgtcactcagtacaattattttgag |
| 2574 | NM_201593 | CACNB2 | ccacaacgagtgcaacaagcagcgcagccgtcataaatccaaggatcgctactgtgaaaa |
| 2575 | R99527 | MGC39372 | tccactgggtcaaaccatacatcgcttcgtctctgtagtgatgacttcctgtcctctgct |
| 2576 | T66903 | LOC54103 | catcctcggggtccagtgtctccctttgcataacctgctgcattgcattgacagtggagt |
| 2577 | U37028 | ITGAD | ctagaagaagacgaggtctacaatgccattcccatcatcatgggcagctctgtgggggct |
| 2578 | W56129 | SFRS9 | gttgctctactatggagatcaacagttactgtgactgagtcggcccattctgtttagaaa |
| 2579 | X58529 | IGHM | atgttaaccctgccgcgtggaatccccccgggaatcgaaattccgaacccaatgcagtcc |
| 2580 | AA188785 | LOC286025 | ttcctccaggtttttcaggggtcacttcacatttctaaagaaagagtgaataggctgggc |
| 2581 | AA347638 | BCR | aacttagagtctaaaagatttctactggatcacttgtcaagatgcgccctctctggggag |
| 2582 | AA601122 | SECTM1 | caagaggggcgcgtttgtgtctcgggttaaaataaggttccgtccgcgtgctgggtcaga |
| 2583 | AA642467 | SLC2A14 | gtcaaacatcacgaagacattggatgtggccccctgtgctgtggtcgcttcttggtcttg |
| 2584 | AA682722 | LARP | acacgtctaccataagtatcgtaggcgctgccttaatgagcggaaacgcttgggcattgg |
| 2585 | AA757235 | LOC399933 | ttggtatgtagaaatatatctgatttgcatactggtcctttatccagcaaccttcttaaa |
| 2586 | AA765313 | FZD3 | ttgcatgactgatagctgtaactcacagttaacatgctttcagtcaagtacagattgtgt |
| 2587 | AB002310 | HUWE1 | accgcactgtgttaaaccagatcctacggcagtccacgacccaccttgctgatgggcctt |
| 2588 | AB002333 | ZNF518 | gccatatcaggaaaataatgtcgagatatgtacttcttgcctttaagttttaacagccat |
| 2589 | AB024402 | ING1 | cgacttgcagagaaatgtctcgctgatgcgggagatcgacgcgaaataccaagagatcct |
| 2590 | AB028952 | SYNPO | ttgagaaaccggcagttcccaagccagcgcctggctgttctctcattgtcactgccctct |
| 2591 | AB032991 | NDFIP2 | gcgggtcgagtgctgtagttcgatttttgcgtgtgtgcgttgcttgcggcctttgtgcct |
| 2592 | AF008915 | EVI5 | gatgctatttatgatccaagtttttacatcacatccattgatctgcctatcccttctttt |
| 2593 | AF108138 | C15orf20 | tgcaaaatgtgcacccatattttaagctgctttttcaggggataaatagtgtttggggac |
| 2594 | AF116668 | LMO2 | aagggatttcttaggggatggtagacctttattgggtatcaagacatagcatccaagtgg |
| 2595 | AF143327 | FLJ32810 | tcaaagcttaacttctgtaggttccaaggagacacccaaagcttcaccaaacccagacct |
| 2596 | AF200348 | D2S448 | gacagagctataaactcaacccgaacacatttgtttgacagccgtcctcgttctccaaat |
| 2597 | AF245480 | MONDOA | ggagatatttctgtaactcattctcttggtgctcacgattgccatggccatagggccaca |
| 2598 | AI075159 | SPTA1 | ttgatgagcggacaaagcttggagactatgccaacctaaaacaattctaccgagaccttg |
| 2599 | BU948323 | TCF4 | cttcgtctgaaaaatgccaatattcaatcatcatgcagcattataacaagccttataagt |
| 2600 | AI675029 | C7 | gttttcagggagtacacaggtagattagtttgaagcattgaccttttatttattccttat |
| 2601 | AI694012 | PSMA5 | agtcaatacctgttagtgttaggctattaacaatagttaggtgaaaggtaatgagggcat |
| 2602 | AI732248 | COL4A1 | tactacgcaaacgcttacagcttttggctcgccaccatagagaggagcgagatgttcaag |
| 2603 | AJ007509 | HNRPUL1 | cttttattccgggcgtcttggtagtttctggtgggattcagtgggtgagagggaagaagg |
| 2604 | AK022224 | ACTN1 | tttctccaatctcatgtccattttggtgtgggagtcggggtagggggtactcttgtcaaa |
| 2605 | AK023928 | PSCD1 | gcacctgggtcagtattagtctatttatcagaggtgtaaataatctatgtatagtttttc |
| 2606 | AK024580 | NRP1 | tcacatttgcatcatagacgagacagtccattcatcttagttaaattggattgagaatgc |
| 2607 | AK025005 | MARCH1 | cttagctgggtcttccactcctaacggaattgagctcgttgatctgggatccaaaggtaa |
| 2608 | AK091853 | COL3A1 | cctcctgggattaatggtagtcctggtggtaaaggcgaaatgggtcccgctggcattcct |
| 2609 | AK126650 | CAPZB | agggacctacacacagaggagtgaacttagggttctagggactatggccgggtcaccggt |
| 2610 | AK127124 | ARHGEF4 | tatccacttgcctggtagaagtagcttgaaggtgagagagataaacaattgtcctcttcc |
| 2611 | AK127132 | DRG1 | ctagcagcttcaacccagtcctccttaccttgcacctattaacgttggttcaacatgaag |
| 2612 | AK129940 | GNAI1 | gaaaaggttagtctttcagtacacgttactggtaagtagtttccaagttacgtgttgtca |
| 2613 | AL049313 | CLIC5 | ggaagaaagtccatgagactttctgactaggaaaccatgtggtttgaacttgaagaaaaa |
| 2614 | AL049327 | CBLB | catgctaattcactctagttcaaatccacattctagttgaatccctgcaatgtaacttga |
| 2615 | AL049449 | GAB1 | ctgtggatttaaacaaattgcgaaaagatgctagttctcaagactgctatgatattccac |
| 2616 | AL050391 | CASP4 | catgatgagggatgggatgaaggcagcaaggggtaaccatcttgaccttctgttttttct |
| 2617 | AL137527 | LRCH3 | ccaactgcgtaaacatattgagtaccggttgaaagtgtctctaccttgtgatctcggagc |
| 2618 | AL353942 | SEPT11 | tctaattggatgtgctttcgcctttgcatgtaagtacggtagtaagaaacctttgagatc |
| 2619 | AL833915 | TDRD9 | agatgaaaacccccatgatggtgaattgaccttcttaggaagagttttagcccaacttcc |
| 2620 | AL834278 | USP30 | gaaagcattcattatgtccggagtgtctttttactcatctgatacaggtaattaaaagaa |
| 2621 | AU252485 | MAP7 | tccaccatgaatctttcgaaatatgttgatcccgtcattagcaagcggctctcctcttca |
| 2622 | AV650179 | PRG1 | tccaacaagatcccccgtctgaggactgacctttttccaaagacgagaatccaggacttg |
| 2623 | AV686223 | KIAA1407 | aaatacatgctgacgtggttccagcgtagtcaggaaagtctggctagaaagatggcccag |
| 2624 | AW014009 | MAIP2K3 | agttgtgtgaataccccaagactcccatgagggagatgccatgagccgcccaaggccttc |
| 2625 | AW190768 | SLC13A3 | cacccccttactctgctataatttctccttaataggacactgtataacttattttcttat |
| 2626 | AW292143 | GFAP | catagtgaccggctgcttttccctaagccaagggcctcttgcggtcccttcttactcaca |
| 2627 | AW439398 | CCND1 | gccaccacggcgttgtacctgtaggactctcattcgggatgattggaatagcttctggaa |
| 2628 | AW452039 | HRMT1L3 | gtttgctccaccagatttaaccaaatgtaactcccacattgagtttatctatattgaaaa |
| 2629 | AW664012 | ALOX5 | gttcacacctgatttttaagacttagaataaggaggtggggaatgtgaaatatctcattt |
| 2630 | AW967479 | CD38 | acaaataagcgagaaccgcgacgagcaccacataagccgtaggaccgcatgaaagaa |
| | | | gag |
| 2631 | AW970654 | PHTF2 | aaacattaccctaatgaggacgcccctaaatcgggtactagttgcagctctcgctgttca |
| 2632 | BC028066 | NALP1 | accaggaaccaccttggcaagatatttacccaccggccatctctgtttactcatgaatgt |
| 2633 | BC034289 | LOC87769 | acttactcttcaacttaatggcagactgtgatcgctaactgcatctggcacaaaaacatg |
| 2634 | BC042366 | PCDH9 | gtgtttaaagagggtcaagtggaggtgcatattccagagaatgctcccgtaggtacctct |
| 2635 | BC045532 | LSM8 | tacttggagatagtcacattagtacataggtgtcctttgcaactgctgtcaactctacat |
| 2636 | BC050383 | TMPO | ggggagataaatcagacttaacatgtatgtaagatcaattcacttaaaagtatggtccaa |
| 2637 | BC050602 | HIST1H2AC | tggtaagtgaactgaaaattccagtcactcttgggctagactcaacgttcttaaaaacta |
| 2638 | BC058855 | VEGF | ttaacatcacgtctttgtctctagtgcagtttttcgagatattccgtagtacatatttat |
| 2639 | BE147267 | ECE1 | agcaggggactgatggctatagcagaatgaggtcgggtcaggaccctcaaacaccatctg |
| 2640 | BE549606 | BZW1 | ttaactgaaaccggtactgatttggaagcagtagctaagtttcttgatgcttctggagca |
| 2641 | BE798965 | LAMC1 | acttacgtacgtggcataacacatcgtgtgagcccatgtatgctggggtagagcaagtag |
| 2642 | BE927772 | SFRS3 | tatccacctggtacctaagtcttgtcatggacttacaggtttgcatgggttccaaatact |
| 2643 | BF055235 | RIPK1 | tgagttctgtttgctccttgtaccaccatccaaatggtgttatcaaatctcttagattcc |
| 2644 | BF513638 | WNT3 | ttttagttctctagggtctgataggaacagacctgaggcttatctttgcacatgttaaag |
| 2645 | BG682263 | HDAC3 | taagagtggcttgggatgctgtgtcccaaggaatttcttttcacctcttggaagggctgg |
| 2646 | BG683220 | PMS2 | gacctctttgataggaatgtttgatagtgatgtcaacaagctaaatgtcagtcagcagcc |
| 2647 | BG747999 | MT1L | ccacggagaatacaacaacgatgcggcacgaggaaaagctggagtcagggaacaacc |
| | | | aga |
| 2648 | BI493513 | PHF8 | ggggttgggagtgttagtaatcaaggtttagaacaccatgagatagttacccctgatctc |
| 2649 | BI559738 | GPR56 | tggttaattctgtccaacaaacacacacgggtagattgctggcctgttgtaggtggtagg |
| 2650 | BM709325 | BCL7A | cccacttctctatgtaacgatataagctatcggagggtggtaccgatcaggaacgctttt |
| 2651 | BM711190 | MED28 | atctcttctgttgccctcgctaggttctccctggtgggttagtgctttgtatctgtctct |
| 2652 | BM727177 | GOLT1B | tttccagttgcactgtatctctggaagtgatgcatgaattcgattggattgtgtcatttt |
| 2653 | BQ009245 | GPM6B | atatgtcacactccttaatagggacctgtgactccttaataaggacctgtgacatgccca |
| 2654 | BQ021469 | AGPS | gattccctggagttcagtactgtaggaggatgggtatctactcgcgcatcaggcatgaag |
| 2655 | BQ215285 | LEF1 | acctaacaaaggcgtaggagtaacctcgcactttgctagctaacctgactccgggtgagg |
| 2656 | BQ898221 | ITGA6 | tcagtattcgggagtaccttggtggatcatcctagtggctattctcgctgggatcttgat |
| 2657 | BU929213 | SESN3 | aatgtaaggcagatgacgtgatctttaagactgctatatatatcagtctcttactctata |
| 2658 | BU954396 | RPL11 | aggctgcattggggccaaacacagaatcagcaaagaggaggccatgcgctggttccag |
| | | | ca |
| 2659 | BX095432 | CNTN5 | ttaagccttcgcagtttccacgtggcattgagcgttcgagtgcacagaactagaagtcaa |
| 2660 | BX640908 | EVI1 | aatccagtgctatccagtccataagccacgtatgacgttatcaaggttgaccagagtggg |
| 2661 | BX648339 | USP53 | gatcatataaacatgaccgagttgtacctcagagtcgagcttctgcacaaataataagtt |
| 2662 | BX648365 | SEPT7 | ctgctaaaatagaatatagcatctttcatatggtaggaaccaacaaggaaactttccttt |
| 2663 | BX649193 | TKT | tgtggcttgttctgcccatgtagactcaggtatatgaggacgttctgagtgggggtttgg |
| 2664 | C05886 | ETV6 | aatccacccggcgtgactcatttcaacactaagtactaggggtgttgtcaggagacaaat |
| 2665 | D31762 | TRAM2 | tcttatcttccatggatgctttaattggaagtgggttgccgacccccttgtgcctagaaa |
| 2666 | D86967 | EDEM1 | ttagccacatggtgagaccgtggtgaaagggggatggaaattgcttggccagtctttgcc |
| 2667 | H41942 | NPEPPS | catttctgggaatggttaaacacaaaaggctgatagctggtatcacatagttggagtcag |
| 2668 | M60028 | HLA-DQB1 | agggggcaaaggggagaacccccacatggtgaagaacttttcccgcgcactttttaaagg |
| 2669 | M77810 | GATA2 | ggctaggacagggtctcggactagggaagtggtttctctgcttaaaaagggtcagggtgg |
| 2670 | N26272 | MSI2 | ttgaatataagattcccaaacagaggagcataacacattcctagtacctaagttccaggt |
| 2671 | N28431 | COL6A1 | cctcagaatagtgatgtgttcgacgttttatcaaaggccccctttctatgttcatgttag |
| 2672 | N41620 | WWP1 | ctgtacagtgaattttccgaacctctcaaagtatgttttccgttcttccacagaaatatg |
| 2673 | N53163 | COL27A1 | tttgccatcagccgggtccagatgaatttcctgcacctgctaagctccgaggtgacccag |
| 2674 | N54946 | EWSR1 | aagcagtctgtcggatcctgtggggatagacgggcatttccctcgttgccccccttttta |
| 2675 | N70000 | UBE3B | ccctcattagcggattccgttccattatcaaacccgagtggatccgaatgttctcaactc |
| 2676 | NM_000016 | ACADM | gatttatgaaggtacttcacaaattcaaagacttattgtagcccgtgaacacattgacaa |
| 2677 | NM_000022 | ADA | cgccttcgacgagcccaaagtagaactgcatgtccacctagacggatccatcaagcctga |
| 2678 | NM_000024 | ADRB2 | ggaagtttacatcctcctaaattggataggctatgtcaattctggtttcaatccccttat |
| 2679 | NM_000033 | ABCD1 | attggattccctattcgtagccatctccgtggccaatgtgactaccgtgccagcagcggg |
| 2680 | NM_000062 | SERPING1 | tcatggagaaattggaattcttcgatttttcttatgaccttaacctgtgtgggctgacag |
| 2681 | NM_000089 | COL1A2 | cttttgccgtataaatagggcagatccgggatttgttattttagcaccacggcagcagga |
| 2682 | NM_000099 | CST3 | ttgaactcaggaaaccctcaaataggccagaacaactcactaattaaattcatcccctcc |
| 2683 | NM_000110 | DPYD | ggctttaaatcctcgaacacaaactcatgcaactctgtgttccacttcggccaagaaatt |
| 2684 | NM_000118 | ENG | catgtccttgatccagacaaagtgtgccgacgacgccatgaccctggtactaaagaaaga |
| 2685 | NM_000120 | EPHX1 | ccttttgagctattgcacacgcctgaaaagtgggtgaggttcaagtacccaaagctcatc |
| 2686 | NM_000147 | FUCA1 | gtcagtgcaaaaacaatgccagagctgtacgaccttgttaacagctataaacctgatctg |
| 2687 | NM_000169 | GLA | ctatgataaaccggcaggagattggtggacctcgctcttataccatcgcagttgcttccc |
| 2688 | NM_000182 | HADHA | gaaaaattgacagcgtatgccatgactattccatttgtcaggcaacaggtttacaaaaaa |
| 2689 | NM_000194 | HPRT1 | gcagtcaattttcacatcaaagacagcatctaagaagttttgttctgtcctggaattatt |
| 2690 | NM_000214 | JAG1 | ggaacaagttgagctatgacttaacatagccaaaatgtgagtggttgaatatgattaaaa |
| 2691 | NM_000234 | LIG1 | ttgctttttagcaaatctgctgtggcaggctgtggattttgagagtcaggggaggggtgt |
| 2692 | NM_000239 | LYZ | ggagtaggaattaagtgaaaggtcacactaccattatttccccttcaaacaaataatatt |
| 2693 | NM_000246 | MHC2TA | tttgtcacacactttggctattagagatcaaccccttcgctcctgtgtcttgcaatggca |
| 2694 | NM_000249 | MLH1 | tggagggactgcctatcttcattcttcgactagccactgaggtgaattgggacgaagaaa |
| 2695 | NM_000251 | MSH2 | tgttgcagagcttgctaatttccctaagcatgtaatagagtgtgctaaacagaaagccct |
| 2696 | NM_000277 | PAH | ctgcacctaatccccataacttccagtatcattttccaattaattatcaagtctgttttg |
| 2697 | NM_000305 | PON2 | gaaagctgctttcgaataaagtgaatacattttgcacaaagtaagcctcacctttgcctt |
| 2698 | NM_000341 | SLC3A1 | ttcgggccttcccgctaaaataagaataaggttaagtaccaattctgccgacaaaggcag |
| 2699 | NM_000361 | THBD | agtgggttacgggagacaacaacaccagctatagcaggtgggcacggctcgacctcaatg |
| 2700 | NM_000382 | ALDH3A2 | tcataaaattggcatcaattttaatgacgctcctggtatggaacctcagatataccctat |
| 2701 | NM_000395 | CSF2RB | acctccagtcccttgagaccccacgtcatgtagagaagttaacggcccaagtggtgggca |
| 2702 | NM_000417 | IL2RA | cgtacgtttcctgagaagtgtctaaaaacaccaaaaagggatccgtacattcaatgttta |
| 2703 | NM_000418 | IL4R | agggaaggcgggaaccttgggttgagtaatgctcgtctgtgtgttttagtttcatcacct |
| 2704 | NM_000435 | NOTCH3 | tgactgggatcccgtgtaccgagtacacgacccaggtatgtaccaagtaggcacccttgg |
| 2705 | NM_000448 | RAG1 | tagggcaaccacttatgagttggtttttgcaattgagtttccctctgggttgcattgagg |
| 2706 | NM_000484 | APP | gccaggcctcatacatactatttactggttctctttctgcgggtgctccaccaagaattc |
| 2707 | NM_000485 | APRT | ccgggtaggtgatgggtaacattccttaaatggtgcatgtcactggcctttcagctggga |
| 2708 | NM_000536 | RAG2 | tcaagcaaccctttggattatgcccatgaacaagttagtttctcatagctttacagatgt |
| 2709 | NM_000546 | TP53 | atctcttgtatatgatgatctggatccaccaagacttgttttatgctcagggtcaatttc |
| 2710 | NM_000561 | GSTM1 | ataagaagaactgtgcggcacagcgcccgctactacagctcgtcacacggtgttgtccga |
| 2711 | NM_000574 | DAF | atattgaagagttctgcaatcgtagctgcgaggtgccaacaaggctaaattctgcatccc |
| 2712 | NM_000578 | SLC11A1 | ccattgtgggctccgacatgcaggaagtcatcggcacggccattgcattcaatctgctct |
| 2713 | NM_000594 | TNF | caggggagttgtgtctgtaatcgccctactattcagtggcgagaaataaagtttgcttag |
| 2714 | NM_000598 | IGFBP3 | acactcagcgggcctaggcacgcgtggctcttgttgccttagctgaaatttctgttgtgc |
| 2715 | NM_000601 | HGF | ctacactggattgatcaactatgatggcctattacgagtggcacatctctatataatggg |
| 2716 | NM_000604 | FGFR1 | gccccagataggtggtgccagtggcttattaattccgatactagtttgctttgctgacca |
| 2717 | NM_000610 | CD44 | gattttgtagccaacattcattcaatactgttatatcagaggagtaggagagaggaaaca |
| 2718 | NM_000628 | IL10RB | ccttttatatcactaaaataagatcatgttttaattgtgagaaacagggccgagcacagt |
| 2719 | NM_000629 | IFNAR1 | tgtctaaaagttaaagcagcactacttacgtcatggaaaattggtgtctatagtccagta |
| 2720 | NM_000640 | IL13RA2 | agactttccatatcaagagacatggtattgactcaacagtttccagtcatggccaaatgt |
| 2721 | NM_000660 | TGFB1 | tgtgacagcagggataacacactgcaagtggacatcaacgggttcactaccggccgccga |
| 2722 | NM_000700 | ANXA1 | gatcctggaaagtaagcgcaaggctactctctaatgctaactcttatgtatgtaagaggt |
| 2723 | NM_000701 | ATP1A1 | ctctaccctggtaggaaagcaccgcagcatgtggggaagcaagacgtcctggaatgaagc |
| 2724 | NM_000717 | CA4 | acctgcgatgagaaggtcgtctggactgtgttccgggagcccattcagcttcacagagaa |
| 2725 | NM_000729 | CCK | agtgcgctctggtcgtctacaccctgaaaccctttgagttcgagtccgcttgctggtgct |
| 2726 | NM_000732 | CD3D | cctcgtaggccacaggggaaaaaggtccatttgtctcaccgggaggctcaatattgcctg |
| 2727 | NM_000773 | CYP2E1 | gcttgtacacaatggacggtatcaccgtgactgtggccgacctgttctttgcggggacag |
| 2728 | NM_000801 | FKBP1A | ccatattgcgcacaaacacccctatcacgaaaattacaccccccaacggcaacttttaca |
| 2729 | NM_000852 | GSTP1 | agcggatgctatggtgccggggcgacaaggtcgcaactataatctaaccgccttgctgct |
| 2730 | NM_000856 | GUCY1A3 | agacgtttagcgggatcatgactatgttgaatatgcagtttgttgtacgagtgaggagat |
| 2731 | NM_000867 | HTR2B | ataagacatttcgggatgcatttggccgatatatcacctgcaattaccgggccacaaagt |
| 2732 | NM_000878 | IL2RB | gtgacatatgtatctatacatccgtatcttttaatgatccgtgtgtaccatctttgtgat |
| 2733 | NM_000885 | ITGA4 | tggctcgccaacgcttcagtgatcaatcccggggcgatttacagatgcaggatcggaaag |
| 2734 | NM_000887 | ITGAX | tcagggcttcatcgtggggctctcagttccgatttcccaggctgaattgggagtgagatg |
| 2735 | NM_000905 | NPY | tcctgcaatgttaaccttggtcagcactcggcacacccctgcgcggtgcacataacagca |
| 2736 | NM_000906 | NPR1 | gtggcgcagaagggttgggggcctgtatgccttgcttctaccatgagcagagacaattaa |
| 2737 | NM_000953 | PTGDR | gcaactgaagcggagactctaaacccagcttgcaggtaagagctttcacctttggtaaaa |
| 2738 | NM_000960 | PTGIR | ctgctgcattgtgggtgatgacgtaggacatgtgcttggtacaaaaagggcctgagacat |
| 2739 | NM_000964 | RARA | ggagcccgtgggtgcacctgttactgttgggctttccactgagatctactggataaagaa |
| 2740 | NM_000967 | RPL3 | tgtagcctactgataactgcaaaggggagcttggaattatgttagtcagtccctgtgaat |
| 2741 | NM_000968 | RPL4 | ttggatctctgggcttttcaagattctgctaagatctgtattaatcatcttgcatggg |
| 2742 | NM_000970 | RPL6 | ggacatatctgtgaaccctccacggtggaaaaaatgccgcccttagtcgggtggggaaaa |
| 2743 | NM_000972 | RPL7A | caaataactggctggcttaacctatgagaagttctatctgacgatcagcttggaacagcc |
| 2744 | NM_001001547 | CD36 | ctgaagagcaaatgaatcctagtacattgaagagtaccgtactctatctggcacttaatt |
| 2745 | NM_001005 | RPS3 | gtgtagtaatgcgtatcagaggtagcggagaatgaagagcgacaagtgggacgagctgta |
| 2746 | NM_001009 | RPS5 | ggtaacaataccgggagcgcaacatacgtacacttctgtaacgcgcactgcacgtgtata |
| 2747 | NM_001015 | RPS11 | aactatttcttagcccatatattcatgtgtcatcgttcaggaacaagtcagtgacaaact |
| 2748 | NM_001019 | RPS15A | attttaatgccgcacatttgcatactatatgcttgttacagtgatccccacagtaactca |
| 2749 | NM_001069 | TUBB2 | cacagacaatactttaattgtaagaggagttccacatcattacatcaacagtgtgaattt |
| 2750 | NM_001079 | ZAP70 | cgataacctcctcatagctgacattgaacttggctgcggcaactttggctcagtgcgcca |
| 2751 | NM_001092 | ABR | ctgatgctccgtgctgttcgggaattgttttatgtacacttgtcaggcagaaaaggtagt |
| 2752 | NM_001130 | AES | gtctgtatatagtcagcttatctcgtgttcaatcgtctgatctctacagagagaagtgga |
| 2753 | NM_001150 | ANPEP | tacaccctgaacccggacttaatccggaagcaggacgccacctctaccatcatcagcatt |
| 2754 | NM_001165 | BIRC3 | cagggacacatttctctgtcttttttgatcagtgtcctatacatcgaaggtgtgcatata |
| 2755 | NM_001175 | ARHGDIB | tgttactttatctcccccaactgatatgttcatagaatgacagtttgtagatagaagatc |
| 2756 | NM_001178 | ARNTL | aagagaatactcgagcgcggagcgattccattgcatttccttccagtagccaggttttga |
| 2757 | NM_001207 | BTF3 | tgtgtcattgtattctttggttctgctcccccactattgaccaatgtatgagatgggaag |
| 2758 | NM_001211 | BUB1B | tcaagatggctgtattgtttggcaccaatatataaactgcttcacccttcaggatcttct |
| 2759 | NM_001219 | CALU | gtgcttctgaggaacaactctaattagtacacttgtgtttgtagatttacactttgtatt |
| 2760 | NM_001226 | CASP6 | cacctgcgcagatagagacaatcttacccgcaggttttcagatctaggatttgaagtgaa |
| 2761 | NM_001228 | CASP8 | ccagacacgtacaaaatccagctatgaatatagagggcttatgattcagattgttatcta |
| 2762 | NM_001229 | CASP9 | ctacacacaaaccagtatcgctcatagatcagcaaaccggggcctactagagtctgaaca |
| 2763 | NM_001237 | CCNA2 | taatacgaaagactggatataccctggaaagtcttaagccttgtctcatggaccttcacc |
| 2764 | NM_001239 | CCNH | aactatacagttgaactaaatatccagcagatacaacaagtagtcagaagttcaaagatg |
| 2765 | NM_001255 | CDC20 | aagcgcaagacgacaggaaacgaagcgagagacgagaagccggtgacgagcagga |
| | | | gaaag |
| 2766 | NM_001256 | CDC27 | ttcgatgatctcccataaacaaatgtaatgggcatattgggactcgtatgtaggaatcaa |
| 2767 | NM_001265 | CDX2 | gccagagtttttaaagccttctggatccatggggggagaagtgatatggtgaagggaagt |
| 2768 | NM_001273 | CHD4 | tagcagtgttatccactacgatgataaggccattgaacggctgctagaccgtaaccagga |
| 2769 | NM_001274 | CHEK1 | cggtgtgcttgggagtggcgattgtgatttacacgacaaaatgccgaggtgctcggtgga |
| 2770 | NM_001280 | CIRBP | gctcaattgaccgtcttttccagacctctttaagtcacactcttaacttagctttctctg |
| 2771 | NM_001295 | CCR1 | acttccactacccaaagacaaaatgtgcccactgtgtgcttttgagtgtattttctttta |
| 2772 | NM_001312 | CRIP2 | gggcagctggttttcttctgagccattagacccaatcgattctgtgttcccaactcggac |
| 2773 | NM_001313 | CRMP1 | ccagtccaacttcagcttatcaggtgcccagatagatgacaacaatcccaggcgcaccgg |
| 2774 | NM_001337 | CX3CR1 | gttccttcaagatcatgtaccccaatttacttgccattactcaattgataaacatttaac |
| 2775 | NM_001344 | DAD1 | taccacaccaaccagacgatcactgcatagtagcacatgacgagtactgcagtcgactcc |
| 2776 | NM_001345 | DGKA | accacagtctgaccgggctgcattgtgtatggtgccacctagagatccacgatgactgcc |
| 2777 | NM_001398 | ECH1 | tgctgcagacccaagacctcgtgaagtcggtccaggccacgactgagaacaaggaactga |
| 2778 | NM_001404 | EEF1G | aacttttagcatcaaacggggcatcttctcacacagtgcacttccgcccaagacagccgg |
| 2779 | NM_001428 | ENO1 | tttccaccaagtgtctagagtcatgtgagcctcgtgtcatctccggggtggccacaggct |
| 2780 | NM_001436 | FBL | acggacctggctagtgacccaaagaccgaagaaaccaagcgaacagccgaagcagat |
| | | | atg |
| 2781 | NM_001441 | FAAH | cccccatgtggcagcccatgggtatgacataggccaaggcccaactaacagtcaagaaac |
| 2782 | NM_001456 | FLNA | gactcaccaagggggatgcagtgcgagatgtggacatcatcgaccaccatgacaacacct |
| 2783 | NM_001462 | FPRL1 | aagagaaacttgtaatggtctctgaaaaggaattgagaagtaattcctctgattctgttt |
| 2784 | NM_001489 | NR6A1 | ggcccttgatggatctaaaactcggaacattactcctcaatgccgtttccccagttgctc |
| 2785 | NM_001513 | GSTZ1 | aaggcatcgactacgagacggtgcccatcaatctcataaaggatgggggccaacagtttt |
| 2786 | NM_001515 | GTF2H2 | cctagaagaatataatggagaaagattttgttatggatgtcagggggaattgaaagacca |
| 2787 | NM_001540 | HSPB1 | tacttttaccctcgcgcgctgattatccaccctgggcgagggataccgatgtgtcccagt |
| 2788 | NM_001560 | IL13RA1 | gtctgcaagtggggtcccagtgtagcaccaatgagagtgagaagcctagcattttggttg |
| 2789 | NM_001607 | ACAA1 | ttgaaagggatactagcctgcagtgggcatctgtcaccgtgtgtgtgcccaagtgggagg |
| 2790 | NM_001630 | ANXA8 | aactacaaattcctgacccaaggacactgtgttataagaggcgtgggctcccctggtggc |
| 2791 | NM_001640 | APEH | ccggaatgtgcctgttcggctcctgctctatcccaaaagcacccacgcattatcagaggt |
| 2792 | NM_001654 | ARAF | tctcaagtctaacaacatcttcctacatgaggggctcacggtgaagatcggtgactttgg |
| 2793 | NM_001664 | RHOA | ggtgggccagacgggttggacatcgttaataatcatagttggcttctaaatactggtagc |
| 2794 | NM_001712 | CEACAM1 | gaggattagcttggagttctctatactcaggtacctctttcagggttttctaaccctgac |
| 2795 | NM_001715 | BLK | cagtcctgaacaccttactggggcttagtactccggatgaccgtgcgaggtcactgttac |
| 2796 | NM_001716 | BLR1 | ctcccttcccataagctatagacccgaggaaactcagagtcggaacggagaaaggtggac |
| 2797 | NM_001728 | BSG | cgccggagtccactcccagtgcttgcaagattccaagttctcacctcttaaagaaaaccc |
| 2798 | NM_001734 | C1S | ttcattaggtccatctgatagtcatgtagggtaggcggaactgataggatttctgtttta |
| 2799 | NM_001743 | CALM2 | tgcaaaacgggtgtattatccaggtactcgtacactatttttttgtactgctggtcctgt |
| 2800 | NM_001747 | CAPG | agcacgcacaaggaacgcaccagatcaaccgacagcagaacgcacgagagccaaga |
| | | | ccat |
| 2801 | NM_001752 | CAT | tagagaatcccactttctatagcagattgtgtaacaattttaatgctatttccccagggg |
| 2802 | NM_001753 | CAV1 | cctcacagttttcatccagccacgggccagcatgtctgggggcaaatacgtagactcgga |
| 2803 | NM_001759 | CCND2 | tatgcgaaaaggtttttaggaagtatggcaaaaatgttgtattggctatgatggtgacat |
| 2804 | NM_001767 | CD2 | cactcaaagccatctgatgctcacatttcatgtagtcagaggaggccatctggtttgaat |
| 2805 | NM_001772 | CD33 | tacattcctgcaatgcatatcattcagacataaagatgggccgaagtacagatacatgct |
| 2806 | NM_001774 | CD37 | ctgctacaacttgtcggcgaccaacgactccacaatcctagataaggtgatcttgcccca |
| 2807 | NM_001776 | ENTPD1 | ctgcccccatattcaacaatctaaccatactgagctgctgattccccagggattgcatat |
| 2808 | NM_001783 | CD79A | ggaaacgacactaggtcgcacagggcatagacgatagtaccattcctggttgaaacacga |
| 2809 | NM_001786 | CDC2 | ttggtcagagtaataactgaaggagttgcttatcttggctttcgagtctgagtttaaaac |
| 2810 | NM_001793 | CDH3 | aggacaccttccgagggagtgtcttagagggagtcctaccaggtacttctgtgatgcaga |
| 2811 | NM_001794 | CDH4 | gggaaggcgcacttggtgctatgagccctccaagtattaaccttacacagagcccccgct |
| 2812 | NM_001795 | CDH5 | tagacaaggacataacaccacgaaacgtgaagttcaaattcaccttgaatactgagaaca |
| 2813 | NM_001798 | CDK2 | ggagcttgttatcgcaaatgctgcactacgaccctaacaagcggatttcggccaaggcag |
| 2814 | NM_001830 | CLCN4 | aagctatgtattaggtccaccatacgatctgtgtaagtaagattatcccacttgcccttt |
| 2815 | NM_001838 | CCR7 | tcaaagccacactctgggctccagagtggggatgacatgcactcagctcttggctccact |
| 2816 | NM_001839 | CNN3 | ttgtgccaatgtatcaatgtagagttgctctgttttcttcaactgtatttattgctgcat |
| 2817 | NM_001897 | CSPG4 | agagtcctggcactggctaggggtgtcaatgcatcagccgtagtgaacgtcactgtgagg |
| 2818 | NM_001901 | CTGF | cagtttatttgttgagagtgtgaccaaaagttacatgtttgcacctttctagttgaaaat |
| 2819 | NM_001905 | CTPS | gaacctcaagtgtgtgttattgagcttggtggaaccgtgggggacatagaaagcatgccc |
| 2820 | NM_001908 | CTSB | aaagatgcttaacaaaggttaccataagccacaaattcataaccacttatccttccagtt |
| 2821 | NM_001916 | CYC1 | cttgaacctcaacacgacagccaacgtgcataagcatgcaacactaccgaggtagccaca |
| 2822 | NM_001923 | DDB1 | tgactgccaagccatgggtagcctgggtgtaaaacctggagatggtggatgatccccacg |
| 2823 | NM_001928 | DF | gacagagcgcagtagtgattagtcgtatttcgcgacacctcacacggtagatgtgatcaa |
| 2824 | NM_001939 | DRP2 | ggactttgccacaaccttaaagaacaaattccgctccaagcattatttcagcaaacaccc |
| 2825 | NM_001941 | DSC3 | ctgtaaccagaagccagttttatctaacggctactgaaacacccactgtgttttgctcac |
| 2826 | NM_001952 | E2F6 | tgttgagtcagtccaaggggttttacttaggacaagttgtaccttgccctctctccagct |
| 2827 | NM_001964 | EGR1 | gtttggcttataaacacattgaatgcgctttattgcccatgggatatgtggtgtatatcc |
| 2828 | NM_001993 | F3 | acatgctttagattatatattccgcacttaaggattaaccaggtcgtccaagcaaaaaca |
| 2829 | NM_001999 | FBN2 | ctgctgttgaacagatcagcctagagagtgtcgacatggacagccccgtcaacatgaagt |
| 2830 | NM_002015 | FOXO1A | gcatttcgctacccgagtttagtaacagtgcagattccacgttcttgttccgatactctg |
| 2831 | NM_002017 | FLI1 | gtcgaagtctacccaatatcccccagtaagagaataaggggcaagaatgactcatgaact |
| 2832 | NM_002023 | FMOD | ctggggagcacttaattcttcccatttcaaaagtaatgttgcctggggcttaacccacc |
| 2833 | NM_002033 | FUT4 | gtaacctcttcaactggacgctctcctaccgggcggactcggacgtctttgtgccttatg |
| 2834 | NM_002046 | GAPD | ccccccacacaaaagcccctttggtgtgaaaaacggcccccacaagtttataaaaggggg |
| 2835 | NM_002049 | GATA1 | caagaagcgcctgattgtcagtaaacgggcaggtactcagtgcaccaactgccagacgac |
| 2836 | NM_002064 | GLRX | cttgaaaactcttctggcgtaaaaacattctcgagttaataatgttaccccttataacag |
| 2837 | NM_002065 | GLUL | agtatgttcaaagtcaaataactcctcattgtaaacaaactgtgtaactgcccaaagcag |
| 2838 | NM_002067 | GNA11 | ttaagttattgacgcccagcgcgcctcgcctcttcacccatcaacgctgtgctttgccca |
| 2839 | NM_002068 | GNA15 | aatctgcagccgctccatggcctcgatcatggcccgcatggacacgaagatgttctggta |
| 2840 | NM_002076 | GNS | tttgacttagaaagtgcccttcaaaaggaccctgttcactgctgcacttttcaatgaatt |
| 2841 | NM_002086 | GRB2 | tcagtgtttaaataaagacctatgtacttaatcctttaactctgcggatagcatttgta |
| 2842 | NM_002090 | CXCL3 | gaaccctcgtaagaaatagtcaaacacattaagtcctttccagctgtccctagaaagctg |
| 2843 | NM_002094 | GSPT1 | ctcccttggcgatctgcaggaacactagtaatgactggaattactccgtgatctttgatg |
| 2844 | NM_002102 | GYPE | catcctgtaacattggcttacctgtatattatatgccacccgtaaccataaaaacaccag |
| 2845 | NM_002107 | H3F3A | tttgagaggtgattgatactgctaacaattttctagtactctagtttgtttcaagaagag |
| 2846 | NM_002108 | HAL | tcggatgctcttggctttaaggatcaatgtcttagccaaaggatacagtggcatttccct |
| 2847 | NM_002110 | HCK | ttgactcttgcaatccacaatctgacattctcaggaagcccccaagttgatatttctatt |
| 2848 | NM_002118 | HLA-DMB | caaggctatatttctgggatgaatataatctgaggaagggagttaaagaccctcctgggg |
| 2849 | NM_002121 | HLA-DPB1 | gttaactattgtataatgtggcctgttatacatgacactcttctgaattgactgtatttc |
| 2850 | NM_002128 | HMGB1 | acccctcttcaatctgaagattagtatggtttggtgttctaacagtatcccctagaagtt |
| 2851 | NM_002129 | HMGB2 | ttgtagctttttgatgggctactcatacagttagattttacagcttctgatgttgaatgt |
| 2852 | NM_002135 | NR4A1 | cggcctttatgttttttgtaagataaaccgtttttaacacatagcgccgtgctgtaaata |
| 2853 | NM_002141 | HOXA4 | tttaagacactgttacagagatactgttgtcaccttctggggcacggtctttggggagag |
| 2854 | NM_002145 | HOXB2 | gcactcggttatcttttaaaattcacacagaaaaattccgtttggtagactccttccaat |
| 2855 | NM_002166 | ID2 | cgactacatcttggacctgcagatcgccctggactcgcatcccactattgtcagcctgca |
| 2856 | NM_002167 | ID3 | acaggaagagagaagaagcgacagccacggaaacaagcaacgccacagagacacga |
| | | | gggc |
| 2857 | NM_002186 | IL9R | cggcctgtgtgaacctgtcgtgcaaagctcacgtcaccaactgctgcagttatctcctga |
| 2858 | NM_002193 | INHBB | tccaagtgccacgtgaactatgcaatttaaagggttgacccacactagacgaaactggac |
| 2859 | NM_002198 | IRF1 | cccctcttattcctctaggcaagcaggacctggcatcatggtggatatggtgcagagaag |
| 2860 | NM_002199 | IRF2 | tctttaggccttgtctgtaaaacgaatggctcaggctaagtgatctctaaatgcctaaat |
| 2861 | NM_002222 | ITPR1 | ataattattattagtgtgggtatggctaatgagttctgattcacccacgaaggttacatt |
| 2862 | NM_002224 | ITPR3 | cttgtacttcattgtgctggtccgcgtgaagaacaagaccgactacacgggccctgagag |
| 2863 | NM_002228 | JUN | aagccgggtagcgcgcgcgagtcgacaagtaagagtgcgggaggcatcttaattaaccct |
| 2864 | NM_002229 | JUNB | aaaatggaacagcccttctaccacgacgactcatacacagctacgggatacggccgggcc |
| 2865 | NM_002230 | JUP | gtaaagtggagccagtgatagtagttaccttgcagggtcgttgtcaggttatgtgagaaa |
| 2866 | NM_002248 | KCNN1 | gaaaaacctgttcccatcaccggcctagcctagaatcctagcctagaagccctctctccc |
| 2867 | NM_002276 | KRT19 | atcacaaccataacccacccacacaaacgatagagactcaataaaacatgtccagctgtc |
| 2868 | NM_002295 | LAMR1 | cagcgcaatggtaggtaggttaacataagatgcctccgtgagaggctggtggtcagccct |
| 2869 | NM_002312 | LIG4 | aacattttgcccgtgaatatgattgctatggtgatagttatttcattgatacagacttga |
| 2870 | NM_002341 | LTB | ggtagatgtataggcagcatgagtggttaagagtcaagggagtagccggggtagtggcttac |
| 2871 | NM_002349 | LY75 | gaagccgtcctgtactgtgccagcaatcacagctttcttgcgactataacatcttttgtg |
| 2872 | NM_002351 | SH2D1A | taagcgaggttccccgtgtgtaggtagatctggtctttagaggcagatagataggtcagt |
| 2873 | NM_002353 | TACSTD2 | gaaggcttaaatgagtttagatgggaaatagcgttgttatcgccttgggtttaaattatt |
| 2874 | NM_002356 | MARCKS | ccaagctgtgataagtggaatggttactgtttatactgtggtatgtttttgattacagca |
| 2875 | NM_002379 | MATN1 | cttgcccttcactcatcgcagttctccccacggctactccaggtcctcttgaaatccgtc |
| 2876 | NM_002388 | MCM3 | ttctctagttcgtcccaaagtcgtccgcagtgtccactactgtcctgctactaagaagac |
| 2877 | NM_002390 | ADAM11 | ggtacggcaaccacatgtcccagatcgtctccaattcgaaaacaaccgtcctgctgtccc |
| 2878 | NM_002392 | MDM2 | gacctactttggtagtggaatagtgaatacttactataatttgacttgaatatgtagctc |
| 2879 | NM_002393 | MDM4 | tctgattagtcaaattattaagtgccatggattactttatgcagcagtcaggtacatagt |
| 2880 | NM_002398 | MEIS1 | ttagagctgcttaaaacaggaagtgatgtataaggtggtggttgttgcatggggacaatg |
| 2881 | NM_002412 | MGMT | agtaggatggatgtttgagcgacacacacgtgtaacactgcatcggatgcggggcgtgga |
| 2882 | NM_002414 | CD99 | caaccaccctagttcctccggtagcttttcagatgctgaccttgcggatggcgtttcagg |
| 2883 | NM_002415 | MIF | cccccgcgaaaacccgcccagcttttattgcccccgttccaaatttctccctatgagtta |
| 2884 | NM_002417 | MKI67 | acttgtactatattggctgccatgatagggttctcacagcgtcatccatgatcgtaaggg |
| 2885 | NM_002419 | MAP3K11 | aagcgcgagatccagggtctcttcgacgagctgcgagccaaggaaaaggaactactga |
| | | | gc |
| 2886 | NM_002421 | MMP1 | accccaaaagcgtgtgacagtaagctaacctttgatgctataactacgattcggggagaa |
| 2887 | NM_002422 | MMP3 | gagaaggggaagcactcgtgtgcaacagacaagtgactgtatctgtgtagactatttgct |
| 2888 | NM_002436 | MPP1 | cgtttttaactgcaactttctataatgccaaaatgacacatctgtgcaatagaatgatgt |
| 2889 | NM_002441 | MSH5 | ccgaaattggggcagtagacgccatcttcacacgaattcatagctgcgaatccatctccc |
| 2890 | NM_002444 | MSN | gaagatcctaccccaattccttgtaggagtataggccggtctaaagtgagctctatgggc |
| 2891 | NM_002447 | MST1R | cacctcgcatgagatgaatgtgcgtccagaacagccgcagttctcacccatgccagggaa |
| 2892 | NM_002460 | IRF4 | gttttacatgccccgtttttgagactgatctcgatgcaggtggatctccttgagatcctg |
| 2893 | NM_002462 | MX1 | tgtgtgtgcgtcctgtcggagccctgtctcctctctctgtaataaactcatttctagcag |
| 2894 | NM_002466 | MYBL2 | cagcctcaccctgtcaggtatcaaagaagacaacagcttgctcaaccagggcttcttgca |
| 2895 | NM_002475 | MLC1SA | cctacacatctcatgacacgcgcccaccctatgcgcgttttcacaaactccgaagaacgc |
| 2896 | NM_002483 | CEACAM6 | ccagtctacttgagttagcataatacagaagtcccctctactttaacttttacaaaaaag |
| 2897 | NM_002512 | NME2 | cttcattccattgacttagaggcaaccaggattgaccattcttttatagagctatttgcc |
| 2898 | NM_002514 | NOV | acggcggtagagggagataactgtgtgttcgatggggtcatctaccgcagtggagagaaa |
| 2899 | NM_002527 | NTF3 | tcactctgtaaaatctgtgtacaccagtattttgcattcagtattgtcaaggccatgact |
| 2900 | NM_002530 | NTRK3 | ggaatatcacttccatacacatagagaactggcgcagtcttcacacgctcaacgccgtgg |
| 2901 | NM_002572 | PAFAH1B2 | atgtgttctcggtcgccgtagacagcgctctggctaccaccgtgaggctacttgaactgt |
| 2902 | NM_002576 | PAK1 | ccattgactaccttctcatgctctgaggtactactgcctctgcagcacaaatttctattt |
| 2903 | NM_002585 | PBX1 | tggcaggatgctactaccccttcatcagtgacctcccctacagaaggccctggcagtgtt |
| 2904 | NM_002592 | PCNA | accgacgaatgacactgacagccgacgaatacccacaacacgctaaacggtagccaca |
| | | | gc |
| 2905 | NM_002600 | PDE4B | tactatcattgtggctttggttcaaaaggaaacactacatttgctcacagatgattcttc |
| 2906 | NM_002608 | PDGFB | tattgtttccctcgtccgtctgtctcgatgcctgattcggacggccaatggtgcttcccc |
| 2907 | NM_002609 | PDGFRB | ggggctttttctttatcaccctcagtcttaatccatccaccagagtctagaaggccagac |
| 2908 | NM_002616 | PER1 | gagtcctccaatcaggacgcactttccggctccagtgacctgctcgaacttctgctgcaa |
| 2909 | NM_002621 | PFC | tttcggaggtaaaagaaagtagctcatgccaacaagtaacattcaccagcatttattgag |
| 2910 | NM_002648 | PIM1 | catcatgagttctgctgaatgccgcgatgggtcaggtaggggggaaacaggttgggatgg |
| 2911 | NM_002658 | PLAU | gtaacagctgaggtctcttgagggagcttagccaatgtgggagcagcggtttggggagca |
| 2912 | NM_002659 | PLAUR | tgactgccagactgtggggaggcactctcctctggacctaaacctgaaatccccctctct |
| 2913 | NM_002687 | PNN | gaattcccggtccgacagaaagaggtctatatcagagagtagtcgatcaggcaaaagatc |
| 2914 | NM_002704 | PPBP | ccaaagtttggaagtgatcgggaaaggaacccattgcaaccaagtcgaagtgatagccac |
| 2915 | NM_002729 | HHEX | agtacctcccaattcaagcagagaaactgacctgactaaagttaatcgcagatgaactag |
| 2916 | NM_002730 | PRKACA | tgaaacagatcgaacacaccctgaatgaaaagcgcatcctgcaagctgtcaactttccgt |
| 2917 | NM_002744 | PRKCZ | acgcccacgtcagggtgaccatctgtggtaaccgagaattccttggaggcagcgtcttca |
| 2918 | NM_002753 | MAPK10 | catgtgatctattactctgacataaacccatctgtaatatattgccagtatataagctgt |
| 2919 | NM_002758 | MAP2K6 | atcttggcagaactttctatatcgcctatagactgatttgcttagatctcttataatttg |
| 2920 | NM_002791 | PSMA6 | acacgcagagagacgtaagcaaaacctaagacgcaaagagcgaagcgactaagaaga |
| | | | agg |
| 2921 | NM_002800 | PSMB9 | gcagttaccatacgagaaatcaaggtcgttgggacggaagtaaccttatctgcttttccc |
| 2922 | NM_002814 | PSMD10 | cttgttttccaggcatcgaataactgttgagattgttctactgttgtcgtatattcttct |
| 2923 | NM_002827 | PTPN1 | taaactcaccttgcttacttcccccttctaaacaacaatcccaacccttacgccatggtg |
| 2924 | NM_002831 | PTPN6 | gtctccccgctggacaatggagacctgattcgggagatctggcattaccagtacctgagc |
| 2925 | NM_002835 | PTPN12 | aatctctaagccacaggaattaagttcagatctaaatgtcggtgatacttcccagaattc |
| 2926 | NM_002844 | PTPRK | gctaaaaaacgcaaagatgccatggggaatacccggcaggagatgactcacatggtgaat |
| 2927 | NM_002856 | PVRL2 | agggttccagactggttggacttgttcgtctggacgacactggagtggaacactgcctcc |
| 2928 | NM_002868 | RAB5B | tcttcctctcccaacataacaatcgtggtaacagaatgcgactgctgatttaccgatgta |
| 2929 | NM_002870 | RAB13 | ttactagatcaggttttagggtcctgcaaaaggctagctcggcactacactagggaattt |
| 2930 | NM_002880 | RAF1 | gatagtggagtcccagcactaccttctttgactatgcgtcgtatgcgagagtctgtttcc |
| 2931 | NM_002887 | RARS | atattgaaggtgaacatgtggcgtatgctgctatgtgaagcagtagctgctgtcatggcc |
| 2932 | NM_002893 | RBBP7 | cccagttggaatactgatagctctaaaaacttagtaaaacctttgttaggattaacagga |
| 2933 | NM_002901 | RCN1 | aaacggagcggagttctggatcatgtcccatccagtccagtgaatccacgacccgcagac |
| 2934 | NM_002908 | REL | gcaaaatgagcaattgagtgactcctttccatatgaattttttcaagtataacttgcaag |
| 2935 | NM_002913 | RFC1 | aaggaagcccaccttactccatactcacttcaagctataaaggcatctagacacagcaca |
| 2936 | NM_002934 | RNASE2 | gacctgtcctagtaacaaaactcgcaaaaattgtcaccacagtggaagccaggtgccttt |
| 2937 | NM_002935 | RNASE3 | tgtgggttgagacactatagagtgtgtcataaccgagaccggataggggagtagttactt |
| 2938 | NM_002936 | RNASEH1 | atgcattagttgtctgtcaaggaaggtgagtggtgaagggtttttacatatatacaagaa |
| 2939 | NM_002953 | RPS6KA1 | ctgtttagcagaactcattctatccccaatcagctccttttccgttctgttctgctggga |
| 2940 | NM_002960 | S100A3 | gccacctggaccccgactgagtttcgggaatgtgactacaacaaattcatgagtgttctg |
| 2941 | NM_002961 | S100A4 | tcgcatacaatcatgtctcacagcgctgatcttagcacattatgtcatgtttccatctca |
| 2942 | NM_002971 | SATB1 | aaaatgtgctttacacagttgcctataaaaagtgctctatgttatccaagcaattcatac |
| 2943 | NM_002972 | SBF1 | aagtctgagcccttccgcatttctccggtcaaccgcatgtatgccatctgccgcagctac |
| 2944 | NM_002975 | CLEC11A | tgctgatctcgtggacatgcttccgctccagctgttagcgacttgagctcgtcttcacag |
| 2945 | NM_002983 | CCL3 | accagttctctgcatcacgtgagtctgagtttcgttgtgggtatcaccactctctggcca |
| 2946 | NM_002984 | CCL4 | cacaaatcaacggttgttatatgtagtttaaaacctaaccagtgtgcggggaatttttct |
| 2947 | NM_002985 | CCL5 | acttcagattctacattaccactcaaacgcgaaacacaatcaaaatacccgagagaccgc |
| 2948 | NM_003002 | SDHD | acaggtgtctaacgggcaatcgcctagaaacgatagagacagaacgcatgcgcgcagg |
| | | | cg |
| 2949 | NM_003005 | SELP | gtgaggctatttcgtgtgagccgctggagagtcctgtccacggaagcatggattgctctc |
| 2950 | NM_003006 | SELPLG | caaggaagatggagctcccccatcccacacgcactgcactgccattgtcttttggttgcc |
| 2951 | NM_003009 | SEPW1 | tcctcctgtcccgtggtttcatcatatctctttgcataccccatgtcttccccagttgtc |
| 2952 | NM_003025 | SH3GL1 | tggaccggtggctagcaatatcgtgtgttgcgcactcgtaaagtgtgttatttcaaccct |
| 2953 | NM_003038 | SLC1A4 | gtcggtagaccaggggttacgtgactggggaaaatctcacatctccttgtctgaaaacat |
| 2954 | NM_003039 | SLC2A5 | gaagacttccccttgacgttgctgtggtctgtaaccgtgtccatgtttccatttggaggg |
| 2955 | NM_003058 | SLC22A2 | tgcttgggaagaatggggatcacaatggcctatgagatagtctgcctggtcaatgctgag |
| 2956 | NM_003072 | SMARCA4 | tcatcatcgtgcctctctcaacgctgtccaactgggcgtacgagtttgacaagtgggccc |
| 2957 | NM_003073 | SMARCB1 | ggaggaccagtcagaccagcgcgtcatcatcaagctgaacatccatgtgggaaacatttc |
| 2958 | NM_003100 | SNX2 | ctcctagaattgaatcaaagagtatgtctgctcccgtgatctttgatagatccagggaag |
| 2959 | NM_003113 | SP100 | tttgaatgagcagacgtacacccgagtagaagggtttgtgcaggacatgcgtctcatctt |
| 2960 | NM_003118 | SPARC | caagtactaggcgtggatatctggataatgaaggagtgtgaattaagaaggagtaccagg |
| 2961 | NM_003120 | SPI1 | ttgggaagttcccaactttcccgttagggtaaaatccgaaattttcccctttttaaaccc |
| 2962 | NM_003121 | SPIB | aggaggaaggaatgccaatgacctagagacacgagaagtccatgtggaggcacacagc |
| | | | ag |
| 2963 | NM_003123 | SPN | cgcttttcataatgtgcaaagtcatcaaagattgaggtggtatgcttgaaagtagcaatt |
| 2964 | NM_003127 | SPTAN1 | tgcaggcctttgccgaccagctcatcgctgccggccattatgccaagggagacatttcta |
| 2965 | NM_003128 | SPTBN1 | caggactatgagcatgtcacgatgttacaagaacgattccgggagtttgcccgagacacc |
| 2966 | NM_003133 | SRP9 | cacatttaaactacctgttaatataagggatttgtagtatcagcttgttgagcaatgact |
| 2967 | NM_003159 | CDKL5 | cccaacccagcaatccgggttctctttcttcgtgagacacgttatgagggaagccctgat |
| 2968 | NM_003189 | TAL1 | gcccagcactttccgtcaacgttgtactgtatgtgatgaattgcgttggtctctgcattt |
| 2969 | NM_003200 | TCF3 | tggcctcgatctactccccggatcactcaagcaataacttctcgtccagcccttctaccc |
| 2970 | NM_003222 | TFAP2C | tctatttaattgaaactctctgttcagaaagcaataactttgtctcgttcctgttgggct |
| 2971 | NM_003225 | TFF1 | agaggaccgccagcgcgcatagagagatcggtgcgcggacagatttgtctcgaacatagt |
| 2972 | NM_003234 | TFRC | gcttgaagatcgttagtatctaacatgtatcccaactcctataattccctatcttttagt |
| 2973 | NM_003262 | TLOC1 | agaggtctgtgaattgtaggttaagaacccctgccctagctaatcagatccaccaatgtt |
| 2974 | NM_003288 | TPD52L2 | ccacaggtcacgctcaggacccgcatctcattccgatcctccttgtgtgggtggtactta |
| 2975 | NM_003302 | TRIP6 | agttcccatcctttgattgatcactctccctgacatccacctgtatgactttgtcaccaa |
| 2976 | NM_003311 | PHLDA2 | taatctttcaccgccccagcgcagacctgggaagcggaaaatcccgccgtattttcccag |
| 2977 | NM_003315 | DNAJC7 | acttacataaaagcctacttgagaagagctcagtgttacatggacacagaacagtatgaa |
| 2978 | NM_003318 | TTK | aatcactggcagattccggagttagcccgaaaagttaatacagagcagaaacataccact |
| 2979 | NM_003352 | SUMO1 | tatgatccaggccgatctacagtcattcaccgggggtaggagagtcacacaagtacaacc |
| 2980 | NM_003361 | UMOD | ttctcaaaatgggacttgtgacggtgtacctgaggcccccatctccttaaagagtgtggc |
| 2981 | NM_003383 | VLDLR | cagtctatggtgccaataaattcactggatcagagcatgccactctagtcaacaacctga |
| 2982 | NM_003390 | WEE1 | gccctactcccacgtatccaacctagtctaccctagaatccactaacgctttattatgtt |
| 2983 | NM_003395 | WNT9A | tgtctgctagctagttggtttctatacaaatacatattcgaatgtcttgctggatgaaga |
| 2984 | NM_003403 | YY1 | agttgctgagtgggttgatctctggtctttccttgacagtgcacgttcgaaggctgtggg |
| 2985 | NM_003415 | ZNF268 | gcatataccttggagggaccatgctatgagggaaagtgtaaatctagaaatgagaaaccc |
| 2986 | NM_003425 | ZNF45 | tttaacttttatggtttggtcgtaggatcagcagggctcgcacttcggtcagcaggaaaa |
| 2987 | NM_003427 | ZNF76 | ttggcaccagggacttcctgacaccacagtcaattaattcctcaggggcctgtggctgga |
| 2988 | NM_003453 | ZNF198 | atgctcttgatacagagttgcttacaatgacggatatgatgagtgaagacgaggggaaaa |
| 2989 | NM_003467 | CXCR4 | agggaactgaacattccagagcgtgtagtgaatcacgtaaagctagaaatgatccccagc |
| 2990 | NM_003480 | MFAP5 | gtcaacaaggagatctgctctcgtcttgtctgtaaggaacacgaagctatgaaagatgag |
| 2991 | NM_003549 | HYAL3 | ttttcctatggagggaaccttgggtaggtttgagcagggggtatagtgtctgctttgcat |
| 2992 | NM_003582 | DYRK3 | ggagcatcttaagaaacaggataaaactggtagtatgaacgttatccacatgctggaaag |
| 2993 | NM_003627 | SLC43A1 | aagggacctggatgacaggcaatcaaggcctgagcaaccaaaaggagtgccccatatg |
| | | | gc |
| 2994 | NM_003636 | KCNAB2 | aaacgagactcagaccgcgacacagccaccgtatttatggaatgacaaaataaataaagc |
| 2995 | NM_003645 | SLC27A2 | gaagattgtgaggaaattttgtaggaaatttgcatacccgtaaagggagacttttttaaa |
| 2996 | NM_003692 | TMEFF1 | cacatggtaattctaagacttgttctttacccatggaatgtaatatttttgcaaagatgg |
| 2997 | NM_003701 | TNFSF11 | agtatcttcaactaatggtgtacgtcactaaaaccagcatcaaaatcccaagttctcata |
| 2998 | NM_003707 | RUVBL1 | aactgttcctgtggttgctttgaaagaacccttccttacctggtgtgttttctataaatc |
| 2999 | NM_003739 | AKR1C3 | tcttctatttcacacaagacttctcccgccgtattggttgcggaccttgcgcggccggag |
| 3000 | NM_003746 | DNCL1 | ggagtgacgcgataaaacgccgaacggaacacgaactgcgacactgaaagaacccac |
| | | | ccg |
| 3001 | NM_003753 | EIF3S7 | tgaccttattgtccgttgtgagcacgatggcgtcatgactggagccaacggggaagtgtc |
| 3002 | NM_003801 | GPAA1 | ggccctaaccctgcgtggcatcaatagcttccgccagtacaagtatgacctggtggcagt |
| 3003 | NM_003805 | CRADD | tcttcctaagtggtagatctaaataccataaatggtggctagctttataggaaaacccat |
| 3004 | NM_003821 | RIPK2 | tttggtttatcaaagtggcgcatgatgtccctctcacagtcacgaagtagcaaatctgca |
| 3005 | NM_003824 | FADD | cgctcttgtcgatttcctgtagtgaatcaggcaccggagtgcaggttcgggggtggaatc |
| 3006 | NM_003842 | TNFRSF10B | tatttgggctacattgtaagatccatctacacagtcgttgtccgacttcacttgatacta |
| 3007 | NM_003852 | TIF1 | caacagaagggtaggttagatgctattaagaaggcacttaatagtacatcatgtaagatg |
| 3008 | NM_003874 | CD84 | aatgaatccatagactcatctagcaccaactaccattagcactatgttaggagctgcaag |
| 3009 | NM_003877 | SOCS2 | gctagaacatttactaatgccaggtgttgcatttgacactttacaagtataacctcactg |
| 3010 | NM_003879 | CFLAR | gacgtcgaggcattacaatcgcgaaaccaagccatagcatgaaacagcgagcttgcagcc |
| 3011 | NM_003884 | PCAF | atgcgtccagggtcataaccccctaaaatccatcatgcaaccttattaatctgtcttggg |
| 3012 | NM_003900 | SQSTM1 | gtccacatgtgaactttttctaggtggcaggacaaatcgcgcccatttagaggatgtggc |
| 3013 | NM_003902 | FUBP1 | agttatttacaattcgtggcactccacaacagatagactatgctcggcaactcatagaag |
| 3014 | NM_003914 | CCNA1 | cacttgccagttgttccggacacatagaaagataacgacgggaagagcggggcccgcttt |
| 3015 | NM_003954 | MAP3K14 | ggtctggaatacctccactcacgaaggattctgcatggggacgtcaaagctgacaacgtg |
| 3016 | NM_003955 | SOCS3 | aacccatgcctcccagctgagcactgggaatgtcagcccagtaagtattggccagtcagg |
| 3017 | NM_003974 | DOK2 | agctgtacgactggccctacaggtttctgcggcgctttgggcgggacaaggtaacctttt |
| 3018 | NM_003998 | NFKB1 | gttgtccctctgctacgttcctattgtcattaaaggtatcacggtcgccacctggcattc |
| 3019 | NM_004039 | ANXA2 | tttatttctcttatcgctccttctaagtacacagacggaagggacaaggagcggaacagg |
| 3020 | NM_004047 | ATP6V0B | tatattaccggctcctccatcattggtggaggagtgaaggcccccaggatcaagaccaag |
| 3021 | NM_004064 | CDKN1B | aagactgatccgtcggacagccagacggggttagcggagcaatgcgcaggaataagga |
| | | | ag |
| 3022 | NM_004067 | CHN2 | gttattaccctgtgtaccttgtccctcattttgctgtgacaccctgaaaaagctgaccac |
| 3023 | NM_004077 | CS | gaaccacactgttgagttggacacactgtaaacccctgggtaactgtcaagtcatgatgg |
| 3024 | NM_004083 | DDIT3 | tgatgtgaccctcaatcccacatacgcagggggaaggcttggagtagacaaaaggaaagg |
| 3025 | NM_004088 | DNTT | caaagcccactttgttcgcagtgtagctgaaatactgtctatctctaataaaaacaggag |
| 3026 | NM_004093 | EFNB2 | gaagagcccgtcgcgctggacagcttacctagtcttgtagcattcggccttggtgaacac |
| 3027 | NM_004099 | STOM | agatgagcgctagccttccaagcatgaagtcggggaccaaattagcctttaactcataaa |
| 3028 | NM_004104 | FASN | gaaatttactgtaactgtcagtgtacacgtctggaccccgtttcatttttacaccaattt |
| 3029 | NM_004111 | FEN1 | tctagttattcagaggagtaagatggtgatgttcacctggcaatcagctgagttgagact |
| 3030 | NM_004119 | FLT3 | tttccttgtgagcaaaagggtcttgataacggatacagcatatccaagttttgcaatcat |
| 3031 | NM_004177 | STX3A | atgatactgatgcggttgagattgctatcgacaacacggcttttatggacgagttctttt |
| 3032 | NM_004208 | PDCD8 | ttgggaaggaggcgggtagagcaccatgatcacgctgttgtgagtggaagattggctgga |
| 3033 | NM_004221 | NK4 | ccgaggagcctaggggagagctttagtgacaaggtcatgagatggttccaggccatgctg |
| 3034 | NM_004235 | KLF4 | caaactgctgcatactttgacaaggaaaatctatatttgtcttccgatcaacatttatga |
| 3035 | NM_004310 | RHOH | gtttttggaattgcttccctttccccacaagaacaaagcactggccgggtaaggtggctc |
| 3036 | NM_004313 | ARRB2 | gaatggggtggtagacttggaagagggatgggctagttggcaggatttcacgtttctgct |
| 3037 | NM_004320 | ATP2A1 | gctcgacgaaatcctcaagttcgttgctcggaactacctagagggataactgttccccct |
| 3038 | NM_004324 | BAX | aactgatcagaaccatcatgggctggacattggacttcctccgggagcggctgttgggct |
| 3039 | NM_004335 | BST2 | tgggagctggggtagtacttcttgtccgcgattctcacgcttaagacctggttttctctt |
| 3040 | NM_004336 | BUB1 | tgcccacttattccagaatggcagtgtattagtaggagagctctacagctatggaacatt |
| 3041 | NM_004343 | CALR | tggacggagacgggtggacttcccgctggatcgaatccaaacacaagtcagattttggca |
| 3042 | NM_004355 | CD74 | ccatagactggaaggtctttgagagctggatgcaccattggctcctgtttgaaatgagca |
| 3043 | NM_004374 | COX6C | tcacagaatttcttcaggttgaattacctagaagtttgtcactgacttgtgttcctgaac |
| 3044 | NM_004380 | CREBBP | ccaagaatacggctctgattgcccccctccaaacacgaggcgtgtgtacatttcttatct |
| 3045 | NM_004385 | CSPG2 | caatctatttaccaggacctgatcgctgcaaaatgaacccgtgccttaacggaggcacct |
| 3046 | NM_004413 | DPEP1 | tgcagacttctttcgggacgaggcagagaggatcatgagggactcccctgtcattgatgg |
| 3047 | NM_004425 | ECM1 | gtgtgccaaacttgtggtaaggttgggttcttgatgccggggggtgtcctttaaccccag |
| 3048 | NM_004430 | EGR3 | gccacagcttgcagcatggtcttgactgaatgtactgttcctgttagcgttacttctcct |
| 3049 | NM_004448 | ERBB2 | agtgttgagggaaaacacatcccccaaagccaacaaagaaatcttagacgaagcatacgt |
| 3050 | NM_004449 | ERG | ggactcaggacatttggggactgtgtacaatgagttatggagactcgagggttcatgcag |
| 3051 | NM_004454 | ETV5 | ttcgcagggataaggggtgcactttatagctatggaaacatgagattctcctctattgga |
| 3052 | NM_004475 | FLOT2 | tcagaggatttccctagagattatgacgttgcagccccgctgcgaggacgtagagacggc |
| 3053 | NM_004494 | HDGF | tcttcctactggtctctctatgcctctctacagtctcgtctcttttaccctggcccctct |
| 3054 | NM_004501 | HNRPU | agtggagggggtaagctaaatcatagtttctgacaataactgggaaggttttttcttaaa |
| 3055 | NM_004508 | IDI1 | tcatccattaagcaatccagccgagcttgaggaaagtgacgcccttggagtgaggcgagc |
| 3056 | NM_004513 | IL16 | ttaagaggcaggctcggatggactatagctttgataccacagccgaagacccttgggtta |
| 3057 | NM_004526 | MCM2 | ccatctatcagaactaccagcgtatccgaatccaggagagtccaggcaaagtggcggctg |
| 3058 | NM_004530 | MMP2 | agtgatggttcccctgttcactctacttagcatgtccctaccgagtctcttctccactgg |
| 3059 | NM_004557 | NOTCH4 | ggcatatgggatgtaagatgttctttcctatatatggtttccaaagggtgcccctatgat |
| 3060 | NM_004583 | RAB5C | gcaccttcctgggcatactctgcgttaaggtggacgttttgaccgagtcgatgcgccgtt |
| 3061 | NM_004615 | TM4SF2 | ctgcttattttaatcacagaagccttaaggggactgatagttgtatgaagggtaggagct |
| 3062 | NM_004624 | VIPR1 | tctgccagaagatcccctcaggactgcaacaggcttgtgcaacaataaatgttggcttgg |
| 3063 | NM_004633 | IL1R2 | gattttaaatgtgttgtccataataccctgagttttcagacactacgcaccacagtcaag |
| 3064 | NM_004642 | CDK2AP1 | gactgtaagtagatctcagttctgcgtttattgtaagttgataaaaacatctggaagaaa |
| 3065 | NM_004688 | NMI | gcacctacatcattgcttctacgcttgacgtggctctgagctcccaatctcctgggacag |
| 3066 | NM_004689 | MTA1 | agatgttggaatatatggtccaatgaagcacatccctcgcgcccgacccgccctggcagt |
| 3067 | NM_004706 | ARHGEF1 | acggcccttcggaaagtgctgtccctgaagcagcttctgtttccggcggaggaagacaat |
| 3068 | NM_004725 | BUB3 | gactgtgccaattccatcggtaccccacgagcatcgcatcacttgccttcagtaatgatg |
| 3069 | NM_004815 | PARG1 | gttccaactctatttttgatccggattacatcaaggagttggtgaatgatatcaggaagt |
| 3070 | NM_004827 | ABCG2 | gtaggttagtagcccttcagtcttaatactttatgatgctatggtttgccattatttaat |
| 3071 | NM_004867 | ITM2A | tgttgagaccaagatctgtcaagagtaagaggcaacagatagagtgtccttggtaataag |
| 3072 | NM_004924 | ACTN4 | tcataatgaagacatagccgattctctgcccgggccccttgctgatgctcctccgggtct |
| 3073 | NM_004936 | CDKN2B | gacaggtttagctgtttcatcagcagcctaatatatactgttaaatttgttaaggatttc |
| 3074 | NM_004938 | DAPK1 | ctatgatgttaaccaacccaacaagcacgggacacctccattactcattgctgctggctg |
| 3075 | NM_004941 | DHX8 | aagtccacagtccgagtgcagaaggccatctgcagtgggttcttccgtaatgctgccaag |
| 3076 | NM_004972 | JAK2 | agaaagcaggtaatcagactggactgtatgtacttcgatgcagtcctaaggactttaata |
| 3077 | NM_004994 | MMP9 | tatttctgccaggaccgcttctactggcgcgtgagttcccggagtgagttgaaccaggtg |
| 3078 | NM_004997 | MYBPH | gcagaaggcagacaaaaagacagggcaatggttcacagtgctggagcgctaccaccca |
| | | | ac |
| 3079 | NM_005012 | ROR1 | taatcatatctgacacttacatagtactccagcttgaatagtactcatgtgcctccgggt |
| 3080 | NM_005080 | XBP1 | ttttaggtcctttagtttgcttctgtaagcaacgggaacacctgctgagggggctctttc |
| 3081 | NM_005084 | PLA2G7 | gacatagattcaaatgcagctattgatcttagcaacaaagcttcattagcattcttacaa |
| 3082 | NM_005085 | NUP214 | ggaacgctcgagtctgcttgctgtgtccaacaaatatggtctggtcttcgctggtggagc |
| 3083 | NM_005098 | MSC | acgtgagatccgcgtcgggacagggttttaagtgacaaagaagggcgagtggcttctctg |
| 3084 | NM_005104 | BRD2 | cgccattacaatccacctccatccgcttggaaatggccttcgtcccggcctatgactggt |
| 3085 | NM_005123 | NR1H4 | ttgaagctatgttccttcgttcagctgagattttcaataagaaacttccgtctgggcatt |
| 3086 | NM_005157 | ABL1 | ctcttgaactgggcgaatgtcttatttaattaccgtgagtgacatagcctcatgttctgt |
| 3087 | NM_005161 | AGTRL1 | ctgccagagagaccgtgaggatgaaattaatagtggggcctttgtgagctagaggctggg |
| 3088 | NM_005178 | BCL3 | aggtgggccgtaacgggcacggatcacgatgtaaattattaagcattttggttggatttc |
| 3089 | NM_005187 | CBFA2T3 | atagtctttgcagaggtccaaatgatattcatgatggtaataaacgagatgtttgccaaa |
| 3090 | NM_005192 | CDKN3 | ttttatataccacgccacccatggggggtcttaacacgtgggggggcacccgataagcag |
| 3091 | NM_005197 | CHES1 | atatacatcaacacttttgctatataacctaagtgataaccctcttttagttacctgcca |
| 3092 | NM_005215 | DCC | gtgataggcttactcatcaaatcatggatctcaaccttgatactatgtattactttcgaa |
| 3093 | NM_005225 | E2F1 | taaaggttttttctgattgaagctttaatggagcgttatttatttatcgaggcctctttg |
| 3094 | NM_005230 | ELK3 | atgttgccaaacgctgccagtatttcatccgcctcacccttctcatctcggtccccgtcc |
| 3095 | NM_005239 | ETS2 | gcctaacagttatggaaactacagtccttataaaccattggcatggtaataaacagatct |
| 3096 | NM_005245 | FAT | tcaattatagatggcaaccaaggaagctcgttcacaattgaccccgtcaggggagaagtc |
| 3097 | NM_005246 | FER | agaaagtttgcaagtaatgttgaaaacgttagcggaagaacttatgcaaacacagcagat |
| 3098 | NM_005248 | FGR | aagggccgaatcccctacccaggcatgaataaacgggaagtgttggaacaggtggagc |
| | | | ag |
| 3099 | NM_005252 | FOS | ctgatcatgcattgttgaggtggtctgaatgttctgacattaacagttttccatgaaaac |
| 3100 | NM_005263 | GFI1 | ccttcccatttccagccctaatctacagacaggaatgcccttcaggtttcttccctcccc |
| 3101 | NM_005318 | H1F0 | cctctggatttttgttcctcctgtacaagaggtgtctttgcttggtttggtggggctgcg |
| 3102 | NM_005334 | HCFC1 | tattgtagagtaacccctgtgactcaatattaccatagtgcgatgtcgttttgtgctatt |
| 3103 | NM_005335 | HCLS1 | ctctgggcttcccagtttgtttacccgggaaagtacgtctagattgtgtggtttgcctca |
| 3104 | NM_005339 | HIP2 | gggtctattctgagccgttaacttcctgtaaggggaaaatgggtgggttaccagaaatac |
| 3105 | NM_005340 | HINT1 | ccatgttcttggaggtcggcaaatgcattggcctcctggttaagcacgttttggggataa |
| 3106 | NM_005356 | LCK | acatgtgacacatatgcaccttgtgtctgtacacgtgtcctgtagttgcgtggactctgc |
| 3107 | NM_005375 | MYB | acccccctcattggtcacaaattgactgttacaacaccatttcatagagaccagactgtg |
| 3108 | NM_005385 | NKTR | aaatgggtcggatagtaagcagtacaatatcatgtgtccctgtaaggaaggttcccaagt |
| 3109 | NM_005424 | TIE1 | atggagcagcctcgaaactgtgacgatgaagtgtacgagctgatgcgtcagtgctggcgg |
| 3110 | NM_005427 | TP73 | gtccaaactgcatcgagtatttcacctcccaagggttacagagcatttaccacctgcaga |
| 3111 | NM_005439 | MLF2 | attcaccgtcagcatatgagccgaatgctgtccggtggctttggatatagccccttcctc |
| 3112 | NM_005494 | DNAJB6 | gcattgtagcacgcgctgtatggttgtgcgttgtagcgtgcgctgtatagttgtgatgcc |
| 3113 | NM_005504 | BCAT1 | gtaatctactgttgggatggtaagaattgatgtaggctgtggtgtaagaatgaattaaaa |
| 3114 | NM_005521 | TLX1 | cacacatggaaacccataggtccacacacaggtggtgttactgtccctcctggtgtcacc |
| 3115 | NM_005529 | HSPG2 | ggtctcaccagctgattctggagaatatgtgtgccgtgtggagaatggatcgggccccaa |
| 3116 | NM_005533 | IFI35 | ctgactttctcgcggcggtagacagcgcctcgcagttgctgtcgacatatttgttcctac |
| 3117 | NM_005556 | KRT7 | actctcaaacaccagcccctctatcggcagcctgtctgaagttctttgaagcccctgtca |
| 3118 | NM_005563 | STMN1 | cacacacacccgcccgaagagcctcagtgcttacactaaggagtcgtcttctatgcaata |
| 3119 | NM_005566 | LDHA | acacaagattaataccatccagcatcaggatatagctgtggattttacaaaccattctta |
| 3120 | NM_005572 | LMNA | gtgcgtgaggagtttaaggagctgaaagcgcgcaataccaagaaggagggtgacctgata |
| 3121 | NM_005583 | LYL1 | catccttttgttaattcttcaggggcccgttcccgactggcgccgttcgtttccgcccat |
| 3122 | NM_005589 | ALDH6A1 | cctagtgtaacaatgaaaccagatttctcacttgctcttcatacttctattttgaggtaa |
| 3123 | NM_005608 | PTPRCAP | tttttaatcccgccaacgcacaatctccggaggaatgcccacgagctctcgcgttggtgg |
| 3124 | NM_005621 | S100A12 | atgctaatcaagatgaacaggtcgactttcaagaattcatatccctggtagccattgcgc |
| 3125 | NM_005651 | TDO2 | gatgctctgatttaattctagaaacaatttgattacctcttgtttgtgacaagactaagc |
| 3126 | NM_005654 | NR2F1 | agacaggagcagcccacccagcagaaatacaatccgagctacaaagcatgggaaaaa |
| | | | gag |
| 3127 | NM_005671 | D8S2298E | gaagaagcttcaaagctcttggaggctttaaagttctttctgttgggtgtgcattacagt |
| 3128 | NM_005687 | FARSLB | aaatacttaattgagctactagtttctctattgtcccagggagatattagacctctcttt |
| 3129 | NM_005730 | CTDSP2 | acttgccccagagaagctactcggccaaacctagccagggtctgttcttgtggaccagag |
| 3130 | NM_005731 | ARPC2 | gtgtacgggagtttcttggtaaatccagaatcaggatacaatgtctctttgctatatgac |
| 3131 | NM_005738 | ARL4A | taggctatttagtcccatagtagttcagatctgcttagatgtgacaggcattgaaggctg |
| 3132 | NM_005761 | PLXNC1 | tgcagccgaaactcatgctgagacgcacggagtccgtcgtcgaaaaactcctcacaaact |
| 3133 | NM_005767 | P2RY5 | gaggaagtgcacccgccgtttttgttcagtctacccactctcagggtaacaatgcctcag |
| 3134 | NM_005781 | TNK2 | gatgcgagcactacctcagaatgacgaccattacgtcatgcaggaacatcgcaaggtgcc |
| 3135 | NM_005794 | DHRS2 | ccagagtgtcaggacatgtgtgacttagcccagattcagactttagtcacaagcaggatc |
| 3136 | NM_005808 | CTDSPL | atatttcatgggaatcgaacctagggatagtgctccacttctgacgatggagtgaagaca |
| 3137 | NM_005809 | PRDX2 | aacttaccgatcatcgcacgccggagttcgatcacgagaccgacggggaactgtcgatct |
| 3138 | NM_005826 | HNRPR | caggaagccgtgaaactgtgtgacagctatgaaattcgccctggtaaacaccttggagtg |
| 3139 | NM_005835 | SLC17A2 | ataacgcagcgtgtgagtctgagcattgcgatcatcgccatggtgaacaccactcagcag |
| 3140 | NM_005845 | ABCC4 | tggttacaaacacttccaatggacagccctcgaccttaactattttcgagacagcactgt |
| 3141 | NM_005870 | SAP18 | gtatgatacctgtacttaagcatcactcaagggtaagtgttctaggaaggttgatttagc |
| 3142 | NM_005895 | GOLGA3 | tccccacaccgtgtgtcacaacaggctagggcacttcacgatgtcactacttgtttttct |
| 3143 | NM_005900 | SMAD1 | actcaaatgggttcacctcataatcctatttcatctgtatcttaaatggccccaggcatc |
| 3144 | NM_005902 | SMAD3 | gctgtctaccagttgacccgaatgtgcaccatccgcatgagcttcgtcaaaggctgggga |
| 3145 | NM_005907 | MAN1A1 | ctatcgagactcatttgatccgcaagtctagcagcggactaacttatatcgcagagtgga |
| 3146 | NM_005911 | MAT2A | tggcagaactacgccgtaatggcactttgccttggttacgccctgattctaaaactcaag |
| 3147 | NM_005923 | MAP3K5 | gaattcggcagcgagtagataatatcgaagtcttgacagcagatattgtcataaatctgt |
| 3148 | NM_005933 | MLL | ctgcttcagcccgattgttttcgccactccattctggaacaaggtttgatatgcacaaaa |
| 3149 | NM_005936 | MLLT4 | agcttaccaacttgagcggcatcgaatagaggcagctatggaccgaaagtctgatagtga |
| 3150 | NM_005944 | CD200 | gggaccacgtctgttaccagcatcctccatatcaaagaccctaagaatcaggtggggaag |
| 3151 | NM_005954 | MT3 | gtttttcaccggggcgtggctaccctgtcgtgacgcgtttccgcgtattgtcccctataa |
| 3152 | NM_005956 | MTHFD1 | agcaaaaaggtgtccctacaggcttcattctgcccattcgcgacatccgcgccagcgttg |
| 3153 | NM_005957 | MTHFR | gtttctccatcagcttatgggatccttgtctttactgacaagaatggaatagaaatgtaa |
| 3154 | NM_005965 | MYLK | aaccaggattcttatttgtgctgttatagaatatcagctctgaacgtgtggtaaagattt |
| 3155 | NM_006005 | WFS1 | tgacatgggtgtgccaggctagactaggaggttccggtgtctggaaaagcactttacaga |
| 3156 | NM_006017 | PROM1 | tcttcacgcagagattttcatctattatactttatcaaagattggccatgttccacttgg |
| 3157 | NM_006038 | SPATA2 | cgtgatgccacgtaacttaccctatggacaaaagcaggaatgactaacacatcctaggag |
| 3158 | NM_006060 | ZNFN1A1 | gattctaatcccaacaagcaagggtctccttcaagattaatgctatcaatcattaaggtc |
| 3159 | NM_006079 | CITED2 | gatgtgggccaaactgttctggatcaggaaagtcatactgttcactttcaagttggctgt |
| 3160 | NM_006082 | K-ALPHA-1 | gccatggtaaatacatggcttgctgcctgttgtaccgtggtgacgtggttcccaaagatg |
| 3161 | NM_006084 | ISGF3G | cctctttgtgataattctcagtagttgtccgtgataatcgtgtcctgaaaatcctcgcac |
| 3162 | NM_006098 | GNB2L1 | gtggaagacagcgtcatggaaggagcacttagccagcgtctctaacgtaaaatggcaaac |
| 3163 | NM_006101 | KNTC2 | tttggaaagttgagtataaacaaaccgacatctgaaagaaaagtctcgctatttggcaaa |
| 3164 | NM_006113 | VAV3 | aacaagggacactcaaactaccagagaaacggaccaatggactgcgaagaactcctaa |
| | | | ac |
| 3165 | NM_006120 | HLA-DMA | cattctttggtgtcccttccccttatgattttactttccacagtgaatggcatttatttt |
| 3166 | NM_006152 | LRMP | tttcctctttttgcctttatctccccaactaaaatacaatggggaagaagtctgcctatg |
| 3167 | NM_006184 | NUCB1 | cccctaccttttgtggaaccagtgatgcctcaaagacagtgtcccctccacagctgggtg |
| 3168 | NM_006185 | NUMA1 | tgggcctggccagtcttcctcttagcctctggatctagaagggaccataagaggagtagg |
| 3169 | NM_006187 | OAS3 | cttaaacagcatggcgctggtacgtaaatagaccaatgcagttaggtggctctttccaag |
| 3170 | NM_006190 | ORC2L | gatttttaccagcagtgtcgggaggcattcctcgtcaatagtgatctgacactccgggcc |
| 3171 | NM_006191 | PA2G4 | caaaattcatcgtcttcaagcccctctttctagccttttctactactctctgcttggtca |
| 3172 | NM_006196 | PCBP1 | ttggatgcatctactcaaaccacccatgaactcaccattccaaataacttaattggctgc |
| 3173 | NM_006209 | ENPP2 | acccagaacaggcttgtgttgtaatagtgtgggccgctttgtctcaaatccgcagttcta |
| 3174 | NM_006216 | SERPINE2 | tggaacagcagtataaggtagtaggtcccggatgagtggtctacattccagtgcagagga |
| 3175 | NM_006230 | POLD2 | aaaagaaaatggcagttatttgtgggacagggctgtgttgagacttggcatgcttctttt |
| 3176 | NM_006231 | POLE | ccttcaagactttgatattcgagcaattctggattgggactactacattgagcggctggg |
| 3177 | NM_006235 | POU2AF1 | gaccccattaagcctaaaggtgatgggtcctcgcctaggcttagtgctaccatgtgggtt |
| 3178 | NM_006255 | PRKCH | gaattgcaggccactcatgtcagtgaccagatttgtggcttataaacattagcagtttat |
| 3179 | NM_006263 | PSME1 | taatgcccgcgcgcgctggtgggcgctttataataaagcggagcatttacacacacccgc |
| 3180 | NM_006268 | DPF2 | gtcttcacaccctgattcctcaagttttctgcttagtggcactgacattaagtagtgggg |
| 3181 | NM_006273 | CCL7 | agctttggagtttgggttttcttgtccaggtgcttcataaagtcctggacccacttctgt |
| 3182 | NM_006286 | TFDP2 | tcttagaattgagattgctacacggagcagtccttgacgatcgtgtttctgacgtttctc |
| 3183 | NM_006299 | ZNF193 | gttctactgttttcagtttcatattacccctatatgcatcaaagctgcaacttcccttgc |
| 3184 | NM_006301 | MAP3K12 | cctgtcttaaggcccagggggaggttaggagactgatagcatgtgatggctcaggctgaa |
| 3185 | NM_006302 | GCS1 | accctacttttgcctgtagcccatatgctagaggttggtgaccctgacgacttggctttc |
| 3186 | NM_006325 | RAN | gtccacatgggaccggaacatttaaggccaggccctagagggtgggtttaagcccccaaa |
| 3187 | NM_006329 | FBLN5 | catgcctggtattttcaaccataaaagaagtttcagttgtccttaaatttgtataacggt |
| 3188 | NM_006343 | MERTK | aaagtttgcctgacgttcggaaccaagcagacgttatttacgtcaatacacagttgctgg |
| 3189 | NM_006354 | TADA3L | gcacctcccctcgcaatcagaacaagcccttcagtgtgccgcatactaagtccctggaga |
| 3190 | NM_006355 | TRIM38 | gtgtgagagaagggttaagactggcttacaatgagatgcttcaaatgaaaagggaattat |
| 3191 | NM_006362 | NXF1 | tacgatacaacccctataccacccgacctaaccgtcggggtgatacttggcatgatcgag |
| 3192 | NM_006427 | SIVA | agcgctcggtgttcacactgaactgtggggtcgacgggaggggtgccttttacatgttct |
| 3193 | NM_006472 | TXNIP | tgtgtgaagttactcgtgtcaaagccgttaggatcctggcttgcggagtggctaaagtgc |
| 3194 | NM_006495 | EVI2B | acaatcatttccaccgcttgactcacttaacttgcccctgccaccagtagattttatgaa |
| 3195 | NM_006509 | RELB | aaggtggggggtaggttggttgttcagagtcttcccaataaagatgagtttttgagcctc |
| 3196 | NM_006534 | NCOA3 | caattttcctctccaaggaatgcacccacgagccaacatcatgagaccccggacaaacac |
| 3197 | NM_006559 | KHDRBS1 | agttaaagtcctaattggatttgtaccgtcctcccattttgttctcggaagattaaatgc |
| 3198 | NM_006565 | CTCF | gtgagctttgcagttacacgtgtccacggcgttcaaatttggatcgtcacatgaaaagcc |
| 3199 | NM_006579 | EBP | acttgtgtacctaagtgaccgccccacctgtggcttatacgtggcgtggcctctttcttc |
| 3200 | NM_006591 | POLD3 | ttggttggagttccatcttgcaagggataatacaaatcctatgatctctatgcccaatat |
| 3201 | NM_006602 | TCFL5 | tggcctgcagcaagccatcgtggttggctggaactagacgtgtgatttgaatgcaggagt |
| 3202 | NM_006676 | USP20 | actcattgctgattggaacaccaggaggaggttggatttctgccagtgggggatgtttct |
| 3203 | NM_006739 | MCM5 | ttgtgaagtgcggaaaaccagaggcgcagtcatgtcgggattcgacgatcctggcatttt |
| 3204 | NM_006747 | SIPA1 | agcctgggaacctctcagagaaggtctctcacttggagtccatgctcaggaagctgcagg |
| 3205 | NM_006748 | SLA | gccctgaatggaactactttaactaatccatagggacttctggtatgctttcctctcttt |
| 3206 | NM_006759 | UGP2 | agcaaaataggaaacggggtcgcataggacaatctctagtgcttgtaaatctaaggaaat |
| 3207 | NM_006762 | LAPTM5 | tcaacgctttaggctgagtcactcctcgggtctctccataattcagcccaacaatgcttg |
| 3208 | NM_006769 | LMO4 | accagaaggtctgctaaaaggtcagagtaatgcagaatgcgtgccttcatctcagatttg |
| 3209 | NM_006803 | AP3M2 | agattttccctcaaagaccatgatggtatcggactagttttcagacactgcctgttgctg |
| 3210 | NM_006820 | IFI44L | ctctataagtctagtgttcatggaggtagcattgaagatatggttgaaagatgcagccgt |
| 3211 | NM_006825 | CKAP4 | gggcagccctgtcggcaaggagcgtgcatactgcgtttgtgtaattgtttgctgtatctc |
| 3212 | NM_006845 | KIF2C | ggtctcgctatcaagatccaacgcagtaatggtttaattcacagtgccaatgtaaggact |
| 3213 | NM_006857 | RY1 | aatcgaaaaggtggattcaacagacctttggatttcattgcatgagaattgaagtgttga |
| 3214 | NM_006875 | PIM2 | aggggaaataaggcttgctgtttgttctcctggggcgctccctccaacttttgcagattc |
| 3215 | NM_006904 | PRKDC | ggcgtgatcgggatcgactttgggcatgcgtttggatccgctacacagtttctgccagtc |
| 3216 | NM_006910 | RBBP6 | tcttccaaagaggctagaacgtcagataaacatgattccactcgtgcttcctcaaataaa |
| 3217 | NM_006931 | SLC2A3 | cctagatctttcttgaagacttgaattagattacagcgatggggacacagaaggtcaccc |
| 3218 | NM_006932 | SMTN | agggggttgtagagcgactgcacataggggaaaacacacttggggtcaggctttttgccc |
| 3219 | NM_006937 | SUMO2 | atgcaagtatactagcagaaaaattgggccatagtatcgtggatttccaggtattttctg |
| 3220 | NM_006938 | SNRPD1 | gaaacgctgagtattcgaggaaataacattcggtattttattctaccagacagtttacct |
| 3221 | NM_006963 | ZNF22 | gatgctttatagtagatcacttaaatactggatcttttgctagtgtgaaaacattgggaa |
| 3222 | NM_006981 | NR4A3 | cttcagttatagtaactgactggtatattcattcagaagcgccataagtcagttgagtat |
| 3223 | NM_006994 | BTN3A3 | aaaatccagtacatggctcgtggagagaagtctttggcctatcatgaatggaaaatggcc |
| 3224 | NM_007022 | CYB561D2 | tatgctttcaggctacaaccactagcacggctgacgatggccctttctgcggagaccgag |
| 3225 | NM_007063 | TBC1D8 | gcccgattacttcaaccaccgagtgatcggggcacaagttgaccagtctgtcttcgagga |
| 3226 | NM_007065 | CDC37 | tgtctgtgtcttctatttggcaaacagcaatgatcttccaataaaagatttcagatgctc |
| 3227 | NM_007079 | PTP4A3 | gcaccttaaattattagaccccggggcagtcaggtgctccggacacccgaaggcaataaa |
| 3228 | NM_007117 | TRH | cgcttatgtcggtgtcccaacgtataaactatcacagtagcgctcatggctcgtaggttt |
| 3229 | NM_012092 | ICOS | tgagccagaggccactaggtattcttgctcccagaggctgaagtcaccctgggaatcaca |
| 3230 | NM_012120 | CD2AP | aatgggatgagggaagtttccagcagatttcaggctgttcttaaagtttttgttggtcat |
| 3231 | NM_012207 | HNRPH3 | caaaaacaccaacatacaagtcttgacaacagcatctggtctactagactttcttacaga |
| 3232 | NM_012223 | MYO1B | gaagcagtcacgaccattgctgcatattggcatgggacccaggtacgtagagaatacagg |
| 3233 | NM_012267 | HSPBP1 | ataaggggggcttacacagttgccctaatgcggctggaatccccattgggggtaggtgag |
| 3234 | NM_012317 | LDOC1 | caaccgcccgtttgatcgccgggcgtggtgattttttgtgagctccgcacacctactttc |
| 3235 | NM_012323 | MAFF | ttattgtgccaatatgccctccaaaccctcccaggattcaaagctaggtttggctgtctg |
| 3236 | NM_012334 | MYO10 | agaaatttgccttgtccctttgtgtacaaccatgcaaaactgtttgttggctcacagaag |
| 3237 | NM_012384 | GMEB2 | attaattcccaatcccccattccaccagggctgggagcccattcccagtgcttcccaagg |
| 3238 | NM_012417 | PITPNC1 | agtgtctgagatatccaggagattcattaggtcatcagcgatcaaaggttagcattttat |
| 3239 | NM_013282 | UHRF1 | tacaggggcaaacagatggaggacggccataccctcttcgactacgaggtccgcctgaat |
| 3240 | NM_013314 | BLNK | gcctgacctgatgaactgttaatatctggtgaggttgagttatcatgctactaatatttt |
| 3241 | NM_013416 | NCF4 | tgaacagttttcactggggttgagatcaaatcacagaacagtgtcaggggccctggaagg |
| 3242 | NM_014005 | PCDHA6 | atttgtctcccgccctaagtcctccagtctccttagagctagtacttactaagcatttac |
| 3243 | NM_014207 | CD5 | aaagctctttcatctatagtttggccaccatacagtggcctcaaagcaaccatggcctac |
| 3244 | NM_014232 | VAMP2 | tctattgtctttttaaatggcacaattttaagggtttgagggtacagtcccttaacctgc |
| 3245 | NM_014244 | ADAMTS2 | gaacaatacaccctctccgcccttggggtaatgtactttgtgatattgccacccgccccc |
| 3246 | NM_014298 | QPRT | aaaggatcaaccacatggggttctgcggtgataatgagcacatagtgaggggtcagcaaa |
| 3247 | NM_014345 | ZNF318 | tgaccactgttcctcagttgaccaccgcttctcagctgaccgctgttcctcagttgacca |
| 3248 | NM_014365 | HSPB8 | ggtgatatacaggtcttatatccccatatggaatttatccatcaaccacataaaaacaaa |
| 3249 | NM_014575 | SCHIP1 | accagcaattatgtgacctgctttcgatcataagactgtgttaagaccttctcctcagtg |
| 3250 | NM_014595 | NT5C | ccccggaaagatgattttgatcaagcacaggtaatcaggactgtcctcaggactttatgt |
| 3251 | NM_014614 | PSME4 | acatgaaagcacccagcgatgtgttgcagaaattatagctggtttaatcagaggttctaa |
| 3252 | NM_014624 | S10OA6 | tcgatcgcgatgtaagtcgcgagccggcaatcgcgtgtgcacatactcgcagatccttct |
| 3253 | NM_014667 | VGLL4 | tgtcagttacagtcctagatatacttactgctggtacagttgtactctaagattggtatt |
| 3254 | NM_014707 | HDAC9 | ctgcaagaaactgggctgctaaataaatgtgagcgaattcaaggtcgaaaagccagcctg |
| 3255 | NM_014724 | ZNF305 | aaatttgagactacctttatattctccaacacaagaccgtgtaggtgtcccttcagagag |
| 3256 | NM_014734 | KIAA0247 | catggagattagctgccgtctcaacgaggataaagacacccacacatcacttggggtccc |
| 3257 | NM_014735 | PHF16 | caaacatcaaaacagtgcttcagccaaattccatatgtaatgccattgggagagtattga |
| 3258 | NM_014780 | CUL7 | aaggaacccaggtcgagatgggacattgtgcggctcatcccacctcagacgtacctgcaa |
| 3259 | NM_014792 | KIAA0125 | acgggcacatacaggaaaacacggtaatgtaattagaatagtcagagaaaagtagccaga |
| 3260 | NM_014824 | FCHSD2 | aagttaaaagcccggcgatctgagtgtaattccaaagctacccacgcaaggaatgattat |
| 3261 | NM_014890 | DOC1 | cagacttttaaggcattccaaatcccagtcttcatgttgaactgggttaagcatttatta |
| 3262 | NM_014899 | RHOBTB3 | tataatattgggaccccataccgttagcccttgtatgtataccaacactgccaaagtaaa |
| 3263 | NM_014914 | CENTG2 | agcagtggagagctcggctcaaagtgcgaagttgtttagatttccacgttacacaaacac |
| 3264 | NM_015050 | KIAA0082 | ccaattctgggtatatcagtgtgtcttgcagaatcttggatcattaaagataaacatatt |
| 3265 | NM_015069 | ZNF423 | atggtctgcttttaaatgtgacttaatctgactgcagtaactagtacagttcaataaagg |
| 3266 | NM_015099 | CAMTA2 | atcccggtggacatgatctcactagccaagcagatcatcgaagccacaccggagcggatt |
| 3267 | NM_015147 | KIAA0582 | ggagaacaccccagttttagaggatgtccttgggaggatcgcaaagcagtctggtgagct |
| 3268 | NM_015149 | RGL1 | gggagaacctactcaagaggtgtctaaattcaaactttccaccaaggtagaaagcacggg |
| 3269 | NM_015158 | ANKRD15 | ctctttgtatttatggattcctttttaccgtgtcacatttactttggtcctctatgtatt |
| 3270 | NM_015166 | MLC1 | aagacatgaacgttttctcttcaccgccgtggggtgtattgactggtcccccatgggctg |
| 3271 | NM_015196 | KIAA0922 | ccaatgtctggactttttggttccatctgggccccgcaaagcgatgtgtatgaaaattgc |
| 3272 | NM_015210 | KIAA0802 | aaagccgaccagccaaataacaggacgtcaccagggatggcccagaaagggtacagt |
| | | | gag |
| 3273 | NM_015261 | KIAA0056 | agaggtgatcagttcagctgttgacgccttgcagaggctttgtagagcatctgcagagac |
| 3274 | NM_015335 | THRAP2 | ggcattttcagtgtactgccagtgcaggcgaccactgcctacacagatccacattaaatc |
| 3275 | NM_015436 | RCHY1 | gatgatccagccgactttgacactgttatctccgtaatagctttaccattaggctaggat |
| 3276 | NM_015458 | MTMR9 | ctgaaacctcaagagtgctgtaaggtacaggtttccatattgtgaacctgtgtacgcaca |
| 3277 | NM_015474 | SAMHD1 | ggaatggaacgacagtacttcagtccaaaatccaactcgcctccgagaagcatccaaaag |
| 3278 | NM_015528 | RNF167 | cttcccctgttatcctggtctaataaccccccacacatacacctctggtgacctatttgc |
| 3279 | NM_015568 | PPP1R16B | cagatctttgatatcgtactgaggtaacttccacgtagccccttgccacgcggcaccggt |
| 3280 | NM_015570 | AUTS2 | aacagccgtaacaggtcaaccttgtggagccatcgcgagttagagggtgaaagatggcag |
| 3281 | NM_015636 | EIF2B4 | catgttgcgctggctaactggcagaaccacgcatccctacggttgttgaatctagtctat |
| 3282 | NM_015670 | SENP3 | agcatattgccaagtatctacaggcagaggcggtaaagaaagaccgactggatttccacc |
| 3283 | NM_015710 | GLTSCR2 | agttgtagctgccatcagatgccggagactcgcccttcaataaaaaatctcttctagctg |
| 3284 | NM_015833 | ADARB1 | cggttaaaagcacagtctatggaacgctaatggagtcagcccctaaagctgtttgctttt |
| 3285 | NM_015855 | WIT-1 | cagatacgtaggcccatgaaattgatgaactgagagttgcttccagtcctgagcgcacca |
| 3286 | NM_015869 | PPARG | agcccaatatttgcttaccaaactggacatcattgatgatctggattcaggcagggtctg |
| 3287 | NM_015965 | GRIM19 | aaatcgaggacttcgaggctcgcatcgcgctgttgccactgttacaggcagaaaccgacc |
| 3288 | NM_016041 | DERL2 | ccaacaccgtcatacatatacctccgctacgcaaccttacctagttacgccccaattgta |
| 3289 | NM_016091 | EIF3S6IP | tccggcggtgctcgcaagcgaggcagccatgtcttatcccgctgatgattatgagtctga |
| 3290 | NM_016184 | CLECSF6 | tgtgtaagggaggtccatagaatttaggtggtctgtcaactattctacttatgagagaat |
| 3291 | NM_016195 | MPHOSPH1 | atatctgtttggattatagctaggatttggagaataagtgtgtacagatcacaaaacatg |
| 3292 | NM_016221 | DCTN4 | aaaggtagacctgctgttaatgatccagcattggtcacaatgtaccaactgctttctgca |
| 3293 | NM_016308 | UMP- | ttatatagtgttattcatggttcagctgatcttaacaaaattcgtagcagtggaaccttg |
| | | CMPK | |
| 3294 | NM_016343 | CENPF | ctgacatcccgacaggaaagactagcccatatatcctgcgaagaacaaccatggcaactc |
| 3295 | NM_016570 | PTX1 | aacctgagctttaggacctttaaaatctcagcatgccttttaagctagtacttttcctgt |
| 3296 | NM_016734 | PAX5 | gaagcatcaagcctggggtaattggaggatccaaaccaaaggtcgccacacccaaagtgg |
| 3297 | NM_017437 | CPSF2 | ttgcgtctttcttgtgactaaccaccctgatatagtattaaccactgtgttcaagagtaa |
| 3298 | NM_017448 | LDHC | ggcggaatggtgtctcagatgttgtgaaaattaacttgaattctgaggaggaggcccttt |
| 3299 | NM_017617 | NOTCH1 | aatgagtctgttgtgtgtcatgccagtgagggacgtcagacttggctcagctcggggagc |
| 3300 | NM_017771 | PXK | aaaattcaagtacacacatcagtgttggttactatgcagagaatgtcattgtgtatagtt |
| 3301 | NM_017787 | C10orf26 | tcttcaatgacaggcttggactagctgtggcccagacatcggccctgcccagaattgcca |
| 3302 | NM_017794 | KIAA1797 | acacatgcctcctaagagaggagtgcattgctttagtacccgggccagttgagactgaaa |
| 3303 | NM_018136 | ASPM | ggcagctgtgatttccctccagtctgcttatcgtggctggaaggttcggaaacagattag |
| 3304 | NM_018209 | ARFGAP1 | tgtgctcgtcccaccctagggactcagccacttgcagaacaggatgggaccgagatttca |
| 3305 | NM_018248 | FLJ10858 | ataagccctcttctaaggcatgtgatgcttgcttgacctcaaggcctattgattcagtgc |
| 3306 | NM_018462 | c3orf10 | cggagaatagagtacattgaagctcgggtgacaaaaggtgagacactcacctagaacagt |
| 3307 | NM_018664 | SNFT | aggggctgttgtgagcccattggtatagcaactagtggtgctgtctactgcaaagcaaat |
| 3308 | NM_018951 | HOXA10 | gtaagcggaataaactagagaagggagacattgtttggatttcctttatactgtgaagtt |
| 3309 | NM_019028 | ZDHHC13 | ggaagacaccatacaatcttggattcatgcagaacctggcagatttctttcagtgtggct |
| 3310 | NM_019071 | ING3 | taaccaagattgccctatagaatggttccattatggctgcgttggattgacagaggcacc |
| 3311 | NM_019102 | HOXA5 | agatcatagttccgtgagcgagcaattcagggactcggcgagcatgcactccggcaggta |
| 3312 | NM_019111 | HLA-DRA | ccaactatacttcgatcaccaatgtacctccagacgtaactgtgctcacaaacagccctg |
| 3313 | NM_019841 | TRPV5 | ctaccatttttgattaacatcgctatcactcttgaccttactcccggttggcctgggggc |
| 3314 | NM_019846 | CCL28 | aacacgaaacatacggccataaaactccttattagagagtctacagataaatctacagag |
| 3315 | NM_019857 | CTPS2 | cgttttccccgtactagatggttagggcgcatagtgccgaactacgctgctgctacagaa |
| 3316 | NM_020310 | MNT | tggctccacggtcatcgcccacacagctaccactcacgcttcagtcatccagactgtgaa |
| 3317 | NM_020368 | SAS10 | gagaatgcatctttgacagttatcttatttgtaaggcagcctataaaatagttctgaagt |
| 3318 | NM_020371 | AVEN | caggaccagccagtttacaagcatgtctcaagctagtgtgttccattatgctcacagcag |
| 3319 | NM_020631 | KIAA0720 | agcaccatgcattatgatgggcatgtccgcttcgaccttcccccacaaggctctgtgctg |
| 3320 | NM_020657 | ZNF304 | attcgaacattctagaggggacatccacaagtctcggtgcagttatgatggtgtgggatg |
| 3321 | NM_020841 | OSBPL8 | tggatccattactcaacccttacctagcagttatttgatcatccgagctacttcagagtc |
| 3322 | NM_020843 | ZNF291 | caagattaccttcttaatggacctcctgatacaccagttgacggtttatgttccagatga |
| 3323 | NM_020892 | DTX2 | tcagccacgatcctctggacagagatttgtggcgaagacctgacgagagactgtaaagga |
| 3324 | NM_020944 | GBA2 | aagacacctttcatcgacatgatcaattctgtacccctaagacagatttatggttgtccc |
| 3325 | NM_020956 | PRX | aggtgcagctgccgaaagtgtcagagattcggctgccggaaatgcaagtgccgaaggttc |
| 3326 | NM_020998 | MST1 | tctcctacccatacttggaggccccgctcagacggtcctgaaaacgtctgaaaggcggtt |
| 3327 | NM_021019 | MYL6 | aaggaccaggcacctatgaggattatgtcgaaggacttcgggtgtttgacaaggaaggaa |
| 3328 | NM_021038 | MBNL1 | cagacagacttgaggtatgtcgagagtaccaacgtggcaattgcaaccgaggagaaaatg |
| 3329 | NM_021071 | DO | cctttttgaccagtgtcatcatcttttccaaaagcagagtataaagaaatcttgtggctc |
| 3330 | NM_021103 | TMSB10 | ctctccgatttgtgtgtgcggcgttgtctttacttgtacgcctcccgtgagaccactctg |
| 3331 | NM_021111 | RECK | caaccctcgatgagaccatcaacaatctccatttccgttttcttggacatcaggattctc |
| 3332 | NM_021114 | SPINK2 | tcacccacctcactatactctcccttatcgcgcgtctatatggccccgatcacccttccg |
| 3333 | NM_021643 | TRIB2 | tggaagacgcctacattctgcggggagatgatgattccctctccgacaagcatggctgcc |
| 3334 | NM_021814 | ELOVL5 | acttatgtgtttgtggagatgttggtgtaaacctactgcactgctaattctataaaagta |
| 3335 | NM_021906 | USP9X | gaaaaactattcctaatcaacatggagtggagagtttattcactgtcttatctgcagaaa |
| 3336 | NM_021949 | ATP2B3 | ttcttctcactctctgtcgtagacacaccgataggaccaatggccaatagatattaaaag |
| 3337 | NM_021960 | MCL1 | tgatcctgttagcaggtggtaataccatgggtgctgtgacactaacagtcattgagaggt |
| 3338 | NM_022067 | C14orf133 | attttgcacaagaacaatgcccctgtgcagatattacaggagtatgtcaatctggtggaa |
| 3339 | NM_022127 | SLC28A3 | agtggatcatctggggaatgccatccccactgtagaaatgctgcctcaaaaatgttctgg |
| 3340 | NM_022161 | BIRC7 | gcaaacctggtcagagccagtgttccctccatgggacctaaagacagtgccaagtgcctg |
| 3341 | NM_022334 | ITGB1BP1 | ggagtccgtttttgaggtcacgcggctagtaattggagcctggtttagaaccaagtcagt |
| 3342 | NM_022366 | TFB2M | ccctggaagataggtagcaactagactgtcgtttttggtggagcggttcatttatttgga |
| 3343 | NM_022436 | ABCG5 | atgcagatgtccacacgaggggtcggagttacctgatcacatcgagagagtgctgggcag |
| 3344 | NM_022438 | MAL | aatgaagactttggatttattaaaaccaagttgtccctaaggaatcagatgtgggcatct |
| 3345 | NM_022469 | GREM2 | tacagaatgcaaacacgagcacactctcttcgaacccaattgtgggtgtagcaatgaaag |
| 3346 | NM_022549 | FEZ1 | cctggtatcacttgctcgctactttctagggtcttaatagtagtcaagaaaagggatctg |
| 3347 | NM_022716 | PRRX1 | ccagccatgcttttgtaacttgccaggtggacttgaccaactacattaccatgctgtgcc |
| 3348 | NM_024006 | VKORC1 | agctcccgcgactgctacgtgctcattagtgttcgtccgacgacgaccgtcatatgtcca |
| 3349 | NM_024319 | C1orf35 | taccagggatggcccctggcttggcctgcgaaggtgaacctgcccagatttatcagtaga |
| 3350 | NM_024408 | NOTCH2 | acagaccatatggatcgtcttccccgggatgtggctcgggatcgcatgcaccatgacatt |
| 3351 | NM_024424 | WT1 | tggaagtcgtttttcctaactaggactcaactatgaggggcgaggagccctttatgaaaa |
| 3352 | NM_024713 | C15orf29 | cagattattgcacgtctgtgatttgagaggtgagttatttaagaggccagttttcaggac |
| 3353 | NM_024728 | C7orf10 | ctctggtattaatgaatctagtgccttttaaatgtatcccacgttttgttccctaccatc |
| 3354 | NM_025263 | PRR3 | tggtggaaactcaagtgtgtctactttgtgagaactgggttgtgcacttaaaactggtgt |
| 3355 | NM_030660 | ATXN3 | catggaatgtaggtgtctgcttcacatcttttagtaggtatagcttgtcaaagatggtga |
| 3356 | NM_030674 | SLC38A1 | cagatgggtgatttaagtgagtcacaagtcacaaaactttgctattcatagttaatcaaa |
| 3357 | NM_030915 | LBH | aatgcctgttgtgagatgaacctcctgtaacttctatctgttcttttttgaggctcaggg |
| 3358 | NM_030926 | ITM2C | tgctaaccgttctcagccctgagccttggagaggagggctgtaacgccttcagtcagtct |
| 3359 | NM_030935 | THG-1 | cagcctggttcacaaatctccagaccccttcggagcagtagcagctcagaagttcagcct |
| 3360 | NM_031243 | HNRPA2B1 | caagacctcattcaattgatgggagagtagttgagccaaaacgtgctgtagcaagagagg |
| 3361 | NM_031423 | CDCA1 | tatttctaaacttgaaaactgctttggagaaataccacgacggtattgaaaaggcagcag |
| 3362 | NM_031966 | CCNB1 | tcttccagttatgcagcacctggctaagaatgtagtcatggtaaatcaaggacttacaaa |
| 3363 | NM_032121 | DKFZp564K142 | acaagttccgtcgccttgtgaaagccccaccgagaaattactccgttatcgtcatgttca |
| 3364 | NM_033238 | PML | accatgtgtgctgttttcagatgtgattgaggggtgttctgccctgcctccactgtcaca |
| 3365 | NM_033512 | TSPYL5 | ttgaggatgttcccaaagttttgtccaatcttatcattagtagattttataagccacaga |
| 3366 | NM_033554 | HLA-DPA1 | tacttggcgtcttagatcccccctcggagtagcggctcacgcaacacaaagtatagcaca |
| 3367 | NM_033642 | FGF13 | aatctgtgttccaaaagtggacatagcatgtacaggcagttttctgtcctgtgcacaaaa |
| 3368 | NM_057168 | WNT16 | gccttttcactgggagaatctggaaaaacctccataaggtatatagcaatctttgatctt |
| 3369 | NM_058195 | CDKN2A | atgtgtcacgatagatacatttcccgtatacttctgcgactatatctctctgtatttcta |
| 3370 | NM_080284 | ABCA6 | tctagttccactgtggaattcctttctggttgtacatttgctaggtagtgacaactaatg |
| 3371 | NM_080700 | TREX2 | gacatcagcaccctagaccagaagctgcgtctggtcacttccgacttccaccagctaatc |
| 3372 | NM_130439 | MXI1 | gttgtgtgttaatgttagactatccctttgtgagtgacactttaacagcattcactgctt |
| 3373 | NM_133259 | LRPPRC | aatccttagagagggtaaccaggaagttccgtttgacgtacctgagttgtggtatgaaga |
| 3374 | NM_133378 | TTN | cgagatgaatacgctccacccaaagccgaactggatgcccgattacacggtgatctggtt |
| 3375 | NM_138714 | NFAT5 | gtgagtttgtctgatgttctaccacaacgtggcgtctgataacagtgagggggggtgggg |
| 3376 | NM_144578 | C14orf32 | ggcactttcactgcctgtcactgatcagcagatactgacttgttgccattaagtgaactt |
| 3377 | NM_144628 | TBC1D20 | ccactcactaccttggcgtcgttttttgggtctgacttgtctcgtcaactgctggtctct |
| 3378 | NM_145698 | ACBD5 | ggatatagtgttcctgctttgtttaataagaacctcatttaaacttgacagctatggtat |
| 3379 | NM_145867 | LTC4S | gtcactagaactttaatgatagagactaggatgcggaagcgagcgcccctccccggggca |
| 3380 | NM_147180 | PPP3R2 | ccttgaactgagagcctgtatctggatttagcacttgaaagatctaactggatatttggg |
| 3381 | NM_152739 | HOXA9 | aatagttacctcaaggcctactgaccaaattgttgtgttgagatgatatttaactttttg |
| 3382 | NM_152788 | EB-1 | aacccaagatgcttgtgcaaaaatgcgggctaactgtcagaagtctacagagcaaatgaa |
| 3383 | NM_152827 | SNX3 | ttcctccaggcttgtaatacccttcacatggaagattaatgagggaaatctttatattct |
| 3384 | NM_152890 | COL24A1 | tttcacacccaggaacctaatcaacttccagtgattgaagtacaaaaacttcctcatctc |
| 3385 | NM_156038 | CSF3R | ttgtggcctataactcagccgggacctctcgtcccactccggtggtcttctcagaaagca |
| 3386 | NM_172020 | POM121 | tcaaccaagtatctgtgcaccccatggtcccttcaagttgacacttgaaattacccatca |
| 3387 | NM_173156 | C1orf16 | gagcccagggctaaaatcagtgctatctacaagccgaaatttaagcaacaactgtgacac |
| 3388 | NM_173216 | SIAT1 | gtacaatgaaggaatcctaattgtatgggacccatctgtataccactcagatatcccaaa |
| 3389 | NM_173609 | C15orf21 | aaggaccagccactttgaacgccttagaggcaaaaagagctttgtgagttgtaagaggaa |
| 3390 | NM_173852 | KRTCAP2 | ggatttctcctggactccatgtgcagatgcctagtatttccattctgaccgttgccctcc |
| 3391 | NM_175739 | SERPINA9 | agctaccgccaccaagttcatagtccgatcgaaggatggtccctcttacttcactgtctc |
| 3392 | NM_175744 | RHOC | caggggccttgtctctcactgcatttggtcaggggggcatgaataaaggctacaggctcc |
| 3393 | NM_178586 | PPP2R5C | tggaccccgttccgtaggcaataacgtgcgtccgcctcagcgcgagattaggagttcaaa |
| 3394 | NM_181311 | TAZ | gtgaacatgagttccgaattcctgcgtttcaagtggggaatcgggcgcctgattgctgag |
| 3395 | NM_181336 | LEMD2 | actttcagtctcgtcctgacacgattccaaaggtctgccatactcttcagaggagttcgg |
| 3396 | NM_181339 | IL24 | gagtatatgtaggaggtgggatatcacttccatgacataagtgctattgcagagccgtgg |
| 3397 | NM_181430 | FOXK2 | tatgtttgtatttggggtgtccctccggctctaggcggcctctgacctgctgtctactcc |
| 3398 | NM_181493 | ITPA | tgtcgtgtccatccttccttcgtctggctcgtgattcacttattcgccctccctactttg |
| 3399 | NM_181702 | GEM | atgcaggtcggggacgcatacctgattgtctactcaatcacagaccgagcgagcttcgag |
| 3400 | NM_182729 | TXNRD1 | cattcctgctactctacctgtatttctcagttgcagcactgagtggtcaaaatacatttc |
| 3401 | NM_182810 | ATF4 | ggaagaaaagggtcccctagttgaggatagtcaggagcgtcaatgtgcttgtacatagag |
| 3402 | NM_183395 | CIAS1 | gagccgaagtggggttcagataatgcacgtgtttcgaatcccactgtgatatgccaggaa |
| 3403 | NM_198232 | RNASE1 | cgcaacatgcaattatattaaactcggccaaaccctctgcgggataaaggggttttgttt |
| 3404 | NM_198400 | NEDD4 | ggtctggaagaaacatgtatcaagcaacacagccccttatcccagatagaaaagtgtctc |
| 3405 | NM_199335 | FYB | tgagctgtgtggtctgtccagctttcacacattgatatgtgaggctacatttgcatgtta |
| 3406 | NM_199423 | WWP2 | aagtggtgtccgcaaagcccaaggtgcataatcgtcaacctcgaattaactcctacgtgg |
| 3407 | NM_201592 | GPM6A | tatggaaattttgtctgaagatcagtggccatattactgtaggccctggttcatgttttc |
| 3408 | NM_201632 | TCF7 | aaagagaccgtctactccgccttcaatctgctcatgcattacccacccccctcgggagca |
| 3409 | NM_201998 | SF1 | cttgtaaagttgccgagggtaggttcatctccaggtttcgggattcccatccgtcctggc |
| 3410 | NM_212474 | FN1 | gggcaactctgtcaacgaaggcttgaaccaacctacggatgactcgtgctttgaccccta |
| 3411 | R14777 | CYFIP2 | ctgctgggccagcagcgtcgctttgacctgttcgacttctgttaccacctgctaaaagtg |
| 3412 | R56397 | PIK3C3 | gaaccagataaaactgtgaaaaaggttcaggataaattccgcttagacctgtcggatgaa |
| 3413 | R59027 | GRP58 | ctcattgaaactctttttgtggatttgatgaccttatggatattgccaaagtttaaggca |
| 3414 | R72151 | GNL3 | gctaaactgttctctgtataagttatggtatgcatgagctgtgtaaattttgtgaatatg |
| 3415 | R79128 | MAP3K1 | gtagcagcaatagtagtaatgctgttatacccagtgacgagacagtgttcaccccagtag |
| 3416 | R86893 | C6orf110 | tcgaagcccgcccgtgttacaacgtggctcgcctaatgttcctcgatgcagagaggaaga |
| 3417 | T07281 | NRIP1 | gcatacatccacagattcactttgtttatgcatatgtagatacaaggatgcacatataca |
| 3418 | T28925 | ITGAL | taatgattgacgtacttagcagctatctctcagtgaactgtgagggtaaaggctatactt |
| 3419 | U60115 | FHL1 | accgtgtagacacacgacatgcaagagttgcagcggctgctccaactcactgctcaccct |
| 3420 | U79271 | AKT3 | tttacattcatctggtttagacttaatatgccacaacgcaccacgaccttcccagggtga |
| 3421 | W04885 | MYL4 | tccgtgtaaccacatgcgcccaatgtccgtttgtctctcccgtgtgcgaaaaaacttgtg |
| 3422 | X66087 | MYBL1 | ggctcattgaacgatggtggtaatatggcgctaaaacatacaccactgaaaacactacca |
| 3423 | AK024272 | | ctttcacctcaagaggttgcgacaagctactccaatacccctgctgggatagtgtcacag |
| 3424 | AK025231 | IGLC2 | aaaatgtcaatggtgtgtctggctggctgatgggatttcacctaattttaatgtggcttt |
| 3425 | AL080190 | | ggctgaaccttatggacagtactgctgatttagtgatctgaccttgaattaggggtttca |
| 3426 | BU618233 | | ccaccatcagtgtatgagactgcctgattccctgtacattcattcctgtggctgctctaa |

TABLE 23
70mer polynucleotide probes, according to one embodiment

| | GenBank | | |
|---|---|---|---|
| | Accession | | |
| SEQ | No. for | | |
| ID NO: | Target gene | Symbol | 70mer |
| 3427 | AA594161 | MYH11 | atttcaccagatccatcctcactcctcgtatcaaggttgggcgagatgtggtacagaaagct |
| | | | cagacaaa |
| 3428 | AA766908 | MME | aatcgagtgagacaaaattttagtccaaataacaagtaccaaagttttatcaagtttgggtct |
| | | | gtgctgc |
| 3429 | AB014540 | SWAP70 | gctttggctaagatagatacttgtgaatcaaagatagcacagaaatgaactaagtatatccc |
| | | | atttggaa |
| 3430 | AF196185 | PARD3 | tggttctgtttcttaatcacgtgcggcggtgtctaagtggtgttaccagtgtacgcgcagtga |
| | | | ccttgga |
| 3431 | AI597616 | MRPL33 | cctgggatggaggaactgagtttgcaaatcagctccatcttcatatcaaggaatagtaaaat |
| | | | aagagaaa |
| 3432 | AI634809 | ARID5B | ccctgttcttcaaagcttagctggtcatatttctacttgcttccttttagcttctgccttgcttgtttta |
| 3433 | AI672553 | AKAP12 | atatgctggactgcattcacacatggcatgaaataagtcaggttctttacaaatggtattttga |
| | | | tagata |
| 3434 | AI809213 | RGS13 | ctctcctttcttcgaatgattcccatagaggatcactgtcagagctcgcctttcttctatctgcc |
| | | | ttata |
| 3435 | AK022231 | STAT1 | aggttaacgttcgcactctgtgtatataacctcgacagtcttggcacctaacgtgctgtgcgt |
| | | | agctgct |
| 3436 | AK022293 | CTSD | aggcacccctagctcttgggatgtttgtatccaactccgggcaatgtggggctacaaaggc |
| | | | ccctggctt |
| 3437 | AK055652 | C3orf6 | tgtataacatgataatgatgcctggtgtattgaaatcctcaataaatactgccaagtgtggatc |
| | | | agttgg |
| 3438 | AL080130 | FLJ14001 | ccacagtggtttctctctagtcagtaacaaaatttcattatggtttcaggcattatatggtggta |
| | | | aataa |
| 3439 | AL833316 | MIR | gcacagttagatgcaatgtttatagaaattgattgttaaaccaaatttacactggcatgtgtgg |
| | | | tgtagt |
| 3440 | AW291384 | STS-1 | atgcctcatagtttacctcagcggagtggtggtttccgagattacgagaaagatgctcccat |
| | | | cactgtgt |
| 3441 | BG993697 | SMYD3 | cttcactccctttcttgaaaacctcctgtagttcctgttgtgtacgggataaaggtgcttaccat |
| | | | ggggc |
| 3442 | BI769730 | HLA-DRB1 | aggtataaaggtcccttacgatacctcgctgtcccgtcatcgtcaatatctaggacggcata |
| | | | gtgaccac |
| 3443 | BQ632574 | RASA1 | gttgaccgctcctccaactaaccagtggtatcacggaaaacttgacagaacgatagcaga |
| | | | agaacgcctc |
| 3444 | BX504817 | SFRS7 | aacttgggaatactggtctgcatcaagtttattcggtagtttgaccgctagtatgttggaagtt |
| | | | atttgg |
| 3445 | H53164 | IRF8 | tgggatgccttactttgcacttaatttaataagggcattctcggaggagtagacgtttaatacg |
| | | | aagtgg |
| 3446 | H57732 | TGFBR2 | ttcctgagagcatgagggcccctagacagagtacaaggtgtaattcagacagccagcca |
| | | | gaaaaagaatt |
| 3447 | L29376 | 3.8-1 | ggttgcaggttgtttaaccttatatgtacagtttcacatatgtataaaaacagtagtttggggg |
| | | | cctctt |
| 3448 | M80899 | AHNAK | ctcctgatttggatattgagggaccagaaggaaagttgaaaggctccaaatttaagatgcc |
| | | | caagttgaa |
| 3449 | NM_000038 | APC | tcgttcgattgccagctccgttcagagtgaaccatgcagtggaatggtaagtggcattataa |
| | | | gccccagt |
| 3450 | NM_000043 | FAS | cctcctgaccaccggggcttttcgtgagctcgtctctgatctcgcgcaagagtgacacaca |
| | | | ggtgttcaa |
| 3451 | NM_000061 | BTK | atgtaaatagccgcacaaaggggtccaacagctctttgagtaggcatttggtagagcttgg |
| | | | gggtgtgtg |
| 3452 | NM_000075 | CDK4 | caggtaccaatagatgggacgacctcggtaaacacatgattcctgctcggaattgcctaac |
| | | | cttccaaac |
| 3453 | NM_000109 | DMD | gacatcaagtgtaattagcttttggagagtgggctgacatcaagtgtaattagcttttggaga |
| | | | gtgggtt |
| 3454 | NM_000189 | HK2 | tctcatcgatttcaccaagcgtggactactcttccgaggccgcatctcagagcggctcaag |
| | | | acaaggggc |
| 3455 | NM_000237 | LPL | agagatgatcgtgcctataaatagtaggaccaatgttgtgattaacatcatcaggcttggaat |
| | | | gaattct |
| 3456 | NM_000269 | NME1 | cacgggatgcatagcgtcgcacgcgatcgggtcgcgtctactaatgcttcgatatctatac |
| | | | gtcgcacac |
| 3457 | NM_000271 | NPC1 | ttctagccctctcgcagggcatcctgactgaactgtgtctaagggtcggtcggtttaccact |
| | | | ggacgggt |
| 3458 | NM_000295 | SERPINA1 | ggatgaggttgagtcataccaaatagtgatttcgatagttcaaaatggtgaaattagcaattc |
| | | | tacatga |
| 3459 | NM_000313 | PROS1 | aggaatcttcttcttggcagctgcagtctgtcaggatgagatatcagattaggttggataggt |
| | | | ggggaaa |
| 3460 | NM_000397 | CYBB | tagtgacagtattgacatctgagcatactccagtttactaatacagcagggtaactgggcca |
| | | | gatgttct |
| 3461 | NM_000405 | GM2A | ccccacggtcccgctttaaggtccataaatatccctaaggaaaaatccaccgtggtacattc |
| | | | agtcctct |
| 3462 | NM_000424 | KRT5 | gaagggataaaaagggggcatcaccgttcctgggtaacagagccaccttctgcgtcctgc |
| | | | tgagctctgt |
| 3463 | NM_000483 | APOC2 | agaagacatacctgcccgctgtagatgagaaactcagggacttgtacagcaaaagcaca |
| | | | gcagccatgag |
| 3464 | NM_000576 | IL1B | tgcccgtcttcctgggagggaccaaaggcggccaggatataactgacttcaccatgcaatt |
| | | | tgtgtcttc |
| 3465 | NM_000579 | CCR5 | ctaaaaccatcatagtacaggtaaggtgagggaatagtaagtggtgagaactactcaggg |
| | | | aatgaaggtg |
| 3466 | NM_000584 | IL8 | gtgctgtgttgaattacggaataatgagttagaactattaaaacagccaaaactccacagtc |
| | | | aatattag |
| 3467 | NM_000585 | IL15 | tttctcttggagttacaagttatttcacttgagtccggagatgcaagtattcatgatacagtaga |
| | | | aaatc |
| 3468 | NM_000600 | IL6 | gcaaagaggcactggcagaaaacaacctgaaccttccaaagatggctgaaaaagatgg |
| | | | atgcttccaatc |
| 3469 | NM_000626 | CD79B | acagccacctatgaggacatagtgacgctgcggacaggggaagtgaagtggtctgtagg |
| | | | tgagcacccag |
| 3470 | NM_000627 | LTBP1 | aagtcctctgaagacaatgagaggatttaggatgagcccgataggtgtggcagaccaaat |
| | | | ggacatttct |
| 3471 | NM_000633 | BCL2 | gaggtggctgatattctgcaacactgtacacataaaaaatacggtaaggatactttacatgg |
| | | | ttaaggta |
| 3472 | NM_000655 | SELL | aaatttcatctcaggcctccctcaaccccaccacttcttttataactagtcctttactaatccaa |
| | | | cccat |
| 3473 | NM_000713 | BLVRB | catgacggcccggataacagcgacaatcgaacgctgaccgcttagaccttcgtgagtag |
| | | | ctcggtgtcgt |
| 3474 | NM_000785 | CYP27B1 | tttgtcttgcccctaggaaggtgaatctgccctagcctggtttacggtttcttataactctccttt |
| | | | gctc |
| 3475 | NM_000788 | DCK | tccctggtgcctctcacttcgttggtgaccagtttcttaaactgaaagctttaatgttacatagt |
| | | | aaatg |
| 3476 | NM_000853 | GSTT1 | aaagagaatataggcccgtggcagatacagaggttttctgcccttttggcctgcatgccca |
| | | | acctttggg |
| 3477 | NM_000877 | IL1R1 | agaaaatgccagaatcgattaaattcattaagcagaaacatggggctatccgctggtcagg |
| | | | ggactttac |
| 3478 | NM_000917 | P4HA1 | cctaccactgcagacgaatgtcagcgtcacataaaaagagcatgtaggatgggacatatt |
| | | | gggatgtatt |
| 3479 | NM_000927 | ABCB1 | atcctcaccaagcggctccgatacatggttttccgatccatgctcagacaggatgtgagttg |
| | | | gtttgatg |
| 3480 | NM_001067 | TOP2A | atcagataggagcagtgacgaaagtaattttgatgtccctccacgagaaacagagccacg |
| | | | gagagcagca |
| 3481 | NM_001068 | TOP2B | tcactggtgtcaagatgtatgagaaggcatatatatagtgaatttcacttcttaacttgttctca |
| | | | agtgt |
| 3482 | NM_001110 | ADAM10 | gacatgtatttcttacgtacactgtacttctgtgtgcaattgtaaacagaaattgcaatatggat |
| | | | gtttc |
| 3483 | NM_001154 | ANXA5 | cgtgtatgtgttggtcatgagcatgctagtatgaataaggcaatgtgttaagcactggcatac |
| | | | aaatgca |
| 3484 | NM_001166 | BIRC2 | attcggaagaacagaatggctctctttcaacaattgacatgtgtgcttcctatcctggataatc |
| | | | ttttaa |
| 3485 | NM_001196 | BID | aaacgaagttgaaaaagtcaagcccctgtgtactggggccggacttcccatcatttgagtg |
| | | | cagcacttt |
| 3486 | NM_001230 | CASP10 | tctgttaattcctggaactgtattttgaatccttaaaggtgagccctcatagggagatccaaa |
| | | | gtcctgt |
| 3487 | NM_001238 | CCNE1 | tccacattatcagttgacagtgtacaatgcctttgatgaactgttttgtaagtgctgctatatcta |
| | | | tcca |
| 3488 | NM_001242 | TNFRSF7 | ttgtcaccatcaggcaaggcacctgaattcccaccgcagagagtgttgtggccgtgagga |
| | | | cttttctgtc |
| 3489 | NM_001254 | CDC6 | ggcactcccaagggcgttggggtcataaggagactataactctacagattgtgaatatattt |
| | | | attttcaa |
| 3490 | NM_001350 | DAXX | tatgtgagctgaaagactgctcttcactgaccggccgtgtcatagagcagcgcatccccta |
| | | | ccgtggcac |
| 3491 | NM_001425 | EMP3 | tgttgtgcgtgttatagatcttgttcccccccgcacctttcccgccgcgttttggcttcttccct |
| | | | ctgtt |
| 3492 | NM_001552 | IGFBP4 | ggtagcagatggagtaatggtcacgaggtccagacccactcccaaagctcagacttgcc |
| | | | aggctcccttt |
| 3493 | NM_001558 | IL10RA | tactcttaggtgccagtctggtaactgaactccctctggaggcaggcttgagggaggattc |
| | | | ctcagggtt |
| 3494 | NM_001618 | PARP1 | aagttgctgatgggtagtacctgtactaaaccacctcagaaaggattttacagaaacgtgtt |
| | | | aaaggttt |
| 3495 | NM_001621 | AHR | tccacagtcagccataataactcctcagacatgttatgctggggccgtgtcgatgtatcagt |
| | | | gccagcca |
| 3496 | NM_001663 | ARF6 | gatgtagttaaattgaacaataaccattggtgactggagcaggtaattatagcctgcagaaa |
| | | | aaattatc |
| 3497 | NM_001706 | BCL6 | tttttcacggaagttttcaatgatgggcgagcgtgcaccatccctttttgaagtgtaggcaga |
| | | | cacaggg |
| 3498 | NM_001760 | CCND3 | gtctagagagggaggcaagccctgttgacacaggtctttcctaaggctgcaaggtttaggc |
| | | | tggtggccc |
| 3499 | NM_001762 | CCT6A | aagaacaggcagaggtaaaaagatgatggaaggtgtggtgactaagggccacggttatt |
| | | | gggtgaaattt |
| 3500 | NM_001770 | CD19 | ttgttgccccgggccacagctcaagacgctggaaagtattattgtcaccgtggcaacctga |
| | | | ccatgtcat |
| 3501 | NM_001771 | CD22 | ggctcagagccagtctttttggtgagggtaaccccaaacctccaaaactcctgcccctgttc |
| | | | tcttccac |
| 3502 | NM_001779 | CD58 | caaagaacaagcattagttttggctgtcatcaacttattatatgactaggtgcttgctttttttgt |
| | | | cagt |
| 3503 | NM_001781 | CD69 | gttggaaaatgtgcaatatgtgatgtggcaaatctctattaggaaatattctgtaatcttcaga |
| | | | cctaga |
| 3504 | NM_001782 | CD72 | aattgtcctcacctaatccagtacctccttacgctttagtcagtcaaatcttgtatttcagcctgt |
| | | | gatg |
| 3505 | NM_001826 | CKS1B | tccccgttataaccgcaccgatacccgcctccgctaatcggatgaaacccctgcacactat |
| | | | tgtcagtaa |
| 3506 | NM_001827 | CKS2 | ttattttatcatcgcgcgaccagctaaacactcctgcggtcgtatggtcgacaacaatcaca |
| | | | cataaaat |
| 3507 | NM_001831 | CLU | ggtaagtattttagcatggctgtcaaggaaattcagagtaaagtcagtgtgattcacttaatg |
| | | | atataca |
| 3508 | NM_001852 | COL9A2 | gtcgaagcgctgaaggaatagggcggctttccttccagcgagcatcattcggctgttacca |
| | | | aaacaaaca |
| 3509 | NM_001853 | COL9A3 | ataaaattcaacgtgaggaagcaagtgacaaggacgcccgaagcacagtggacggtcat |
| | | | gaaggagcggg |
| 3510 | NM_001877 | CR2 | atggctctaaaagttttgccctttttaaggaggcactaaaaagagctgtcctggtatctagac |
| | | | ccatctt |
| 3511 | NM_001888 | CRYM | cttaaatgctgagatcctcatttatgtttgtagttggaaagcaaagctaggtagccatttcttct |
| | | | gttct |
| 3512 | NM_001894 | CSNK1E | ctctgcaaaggctatccctccgaattctcaacatacctcaacttctgccgctccctgcggttt |
| | | | gacgaca |
| 3513 | NM_001951 | E2F5 | gacaaaagaaataccagatcaatctaaagagtcattcaggacctatccatgtgctgcttata |
| | | | aataaaga |
| 3514 | NM_001967 | EIF4A2 | ggctcgtggccggaacccgtttcttgcggaggaattttctcgccgtgcttcggtcagctaca |
| | | | aaattgtg |
| 3515 | NM_002002 | FCER2 | accaagcatgccagccacaccggctcctggattggccttcggaacttggacctgaaggg |
| | | | ggagtttatct |
| 3516 | NM_002051 | GATA3 | tttttaacatcgacggtcaaggcaaccacgtcccgccctactacggaaactcggtcaggg |
| | | | ccacggtgca |
| 3517 | NM_002095 | GTF2E2 | tcctggttcctttcttcctcgtttgttactttagagcaagtttgcccatagtcttgaatgcaatattt |
| | | | gt |
| 3518 | NM_002101 | GYPC | cccatctgcagtgagtaggaagaggtcccctcaacagaggtgagaggttgggccaagtc |
| | | | aagtcagtttg |
| 3519 | NM_002105 | H2AFX | cttgattggtcacgatccccgcctacccaccaatccttaacgcgcctcaccggaccttaac |
| | | | attccaccc |
| 3520 | NM_002120 | HLA-DOB | agctaagggctcagaaaggatatgtgaggacgcagatgtctggtaatgaggtctcaagag |
| | | | ctgttctgct |
| 3521 | NM_002122 | HLA-DQA1 | caaagacatgcctggggtaagccacccggctacctaattcctcagtaacctccatctaaaa |
| | | | tctccaagg |
| 3522 | NM_002189 | IL15RA | gttgaacaaggccacgaatgtcgcccactggacaacccccagtctcaaatgcattagaga |
| | | | ccctgccctg |
| 3523 | NM_002211 | ITGB1 | tttgagttgcccatcttgtttcacactagtcacattcttgttttaagtgcctttagttttaacagttc |
| | | | ac |
| 3524 | NM_002217 | ITIH3 | ctgattgatggtgtccacactgactacattgtccccaacctgttttgagtagacacaccagct |
| | | | cctgttg |
| 3525 | NM_002250 | KCNN4 | gttaatgccactgggcacctttcagacacactttggctgatccccatcacattcctgaccatc |
| | | | ggctatg |
| 3526 | NM_002305 | LGALS1 | catggaggccatcaactacatggcggcggatggagacttcaagattaagtgcgtggccttt |
| | | | gagtgaagc |
| 3527 | NM_002350 | LYN | atctattctctccaaaaatgcacccaactagctctatgtttacaaatggacataggactcaaa |
| | | | gtttcag |
| 3528 | NM_002358 | MAD2L1 | ctcgcgcgtacatgttcgtgccagaacatgttacaccactcctacgatggcttgcccgattc |
| | | | gatgcccc |
| 3529 | NM_002426 | MMP12 | gactctactattaagtttgaaaatagttaccttcaaaggccaagagaattctatttgaagcatg |
| | | | ctctgt |
| 3530 | NM_002467 | MYC | cagaaatgtcctgagcaatcacctatgaacttgtttcaaatgcatgatcaaatgcaacctca |
| | | | caaccttg |
| 3531 | NM_002526 | NT5E | tgaattcattctactcatactacacacccagttatggaatgtccagagttctcgaagaaaata |
| | | | aatgact |
| 3532 | NM_002567 | PBP | gagctgggcaaagtgccgacgcccacccaggttaagaatagacccaccagcatttcgtg |
| | | | ggatggtcttg |
| 3533 | NM_002646 | PIK3C2B | gtatcaatgatgcaattactaggctcaacttgaaatcgacctatgatgtggagatgttgcgg |
| | | | gatgccac |
| 3534 | NM_002661 | PLCG2 | agcctgccatcaaggacatttcttaagacccaactggcatgagttggggtaatttcctattatt |
| | | | ttcatc |
| 3535 | NM_002692 | POLE2 | ccatcttaccaaggccaccacttgctgaaagcatcactaatgaattcagacaaagggtacc |
| | | | attttcagt |
| 3536 | NM_002738 | PRKCB1 | aggattcacggtgcacatgctggcattcaacatgtggaaagcttgtcttagagggcttttctt |
| | | | tgtatgt |
| 3537 | NM_002752 | MAPK9 | tgacaagcggatctctgtagacgaagctctgcgtcacccatacatcactgtttggtatgacc |
| | | | ccgccgaa |
| 3538 | NM_002832 | PTPN7 | aatgcttctgcttggccttatggaggagctgctccttccttacagccttggggatggacttgc |
| | | | ccacacc |
| 3539 | NM_002838 | PTPRC | ccgttatgttgacattcttccttatgattataaccgtgttgaactctctgagataaacggagatg |
| | | | caggg |
| 3540 | NM_002891 | RASGRF1 | gcgcaaggacggcacgcgcaaaggctacctgagcaagcggagttcggacaacacaaa |
| | | | atggcaaaccaag |
| 3541 | NM_002892 | ARID4A | gataagaccagcattgaggatgtagcagttgaaagctctgagtctaactctcttgtttctattc |
| | | | cacctg |
| 3542 | NM_002943 | RORA | ataaaaaccagttccataacaatactgaggcattgtttcccctttttactacggtgaccatttgt |
| | | | attaa |
| 3543 | NM_002966 | S100A10 | tccgccgccactcataccaactatcggcaagaacgcaccgtttccgcacttcatttttaccc |
| | | | cgcgcttc |
| 3544 | NM_002967 | SAFB | tgggctaaatgtgccgtgggttccacgccgtgtgcgcaagttccctgtgtgaaagcacgtc |
| | | | tgtcttcca |
| 3545 | NM_003037 | SLAMF1 | gcttttggggcaagctacggaacaggtgggcgcatgatgaactgcccaaagattctccgg |
| | | | cagttgggaa |
| 3546 | NM_003088 | FSCN1 | tgctactttgacatcgagtggcgtgaccggcgcatcacactgagggcgtccaatggcaag |
| | | | tttgtgacct |
| 3547 | NM_003177 | SYK | ggctgcaagaaatgtgttgctagttacccaacattacgccaagatcagtgatttcggactttc |
| | | | caaagca |
| 3548 | NM_003217 | TEGT | cttgagaacttactaaaggcacttccttcctgttaaacccctgttaactctccataaatttggtg |
| | | | attct |
| 3549 | NM_003243 | TGFBR3 | tgcgtttgcagcctttgtgatcggagcactcctgacgggggccttgtggtacatctattctca |
| | | | cacaggg |
| 3550 | NM_003254 | TIMP1 | cctaaggcttggaaccctttatacatcttggtcatcttgatctcataacgctggtataaggtgg |
| | | | tctggt |
| 3551 | NM_003290 | TPM4 | gccccctctccaattcatgtgttgaagccctcgtctccatgtgatggtaattggtgttggggc |
| | | | ctttgga |
| 3552 | NM_003299 | TRA1 | ttgggagagacttgttttggatgccccctaatccccttctcccctgcactgtaaaatgtggga |
| | | | ttatggg |
| 3553 | NM_003332 | TYROBP | ctacagcgacctcaacacacagaggccgtattacaaatgagcccgaatcatgacagtca |
| | | | gcaacatgata |
| 3554 | NM_003362 | UNG | ggttaagattgctgtgagctttatcagataagagaccgagagaagtaagctgggtcttgttat |
| | | | tccttgg |
| 3555 | NM_003451 | ZNF177 | agaaaggatactaaatgaaacaacaggggagatacaatattatctggttgcactaatcctgt |
| | | | gctgctta |
| 3556 | NM_003648 | DGKD | aaacgcctcttgtattgtcatgtacatagtccatacctgagtgctgtacataagttgttctgtgt |
| | | | ataaa |
| 3557 | NM_003656 | CAMK1 | aggttgggccactcacagctgcatgatgaggtagaggtggcccccactctcatagatgtc |
| | | | atccagggct |
| 3558 | NM_003810 | TNFSF10 | agggacaacatccttaagtcaaaagagagaagaggcaccactaaaagatcgcagtttgc |
| | | | ctggtgcagtg |
| 3559 | NM_003875 | GMPS | tgccagagatctgtggttattcgaacctttattactagtgacttcatgactggtatacctgcaa |
| | | | cacctg |
| 3560 | NM_003959 | HIP1R | tttaatattccgagctagagctcttcttcctacgtttgtagtcagcacactgggaaaccgggc |
| | | | cagcgtg |
| 3561 | NM_004049 | BCL2A1 | gatgtggatacctataaggagatttcatattttgttgcggagttcataatgaataacacagga |
| | | | gaatgga |
| 3562 | NM_004073 | PLK3 | tgtggttcagcgaatgggttggcttctccaataagttcggctttgggtatcaactgtccagcc |
| | | | gccgtgt |
| 3563 | NM_004091 | E2F2 | tggtcgtgtctttatgagcaccatgtaagcctccttgtattgagataattgggcattaaacatta |
| | | | aactg |
| 3564 | NM_004117 | FKBP5 | gggtgtgtgagtggttctgagcaaataactacagggtgcccattaccactcaagaagaca |
| | | | cttcacgtat |
| 3565 | NM_004126 | GNG11 | tgacctttccccggagactttcttaagcaccatatagatacggttatgtttttaaagcctatgtg |
| | | | cctac |
| 3566 | NM_004184 | WARS | gtttcttcctgtgagttccattatttctatctcttatgggcaaagcattgtgggtaattggtgctg |
| | | | gcta |
| 3567 | NM_004271 | LY86 | ccaaacataggaagcggcaatcagcccgcgcgtggaaccgagacaagtaaacaagac |
| | | | gccagatctctga |
| 3568 | NM_004289 | NFE2L3 | ctttccatcttggcagccatcctttttaagagtaagttggttacttcaaaaagagcaaacactg |
| | | | gggatc |
| 3569 | NM_004322 | BAD | agctggagacctagcacccagaagatgagaccgcaattcgagaatcaaagggccggga |
| | | | gcgatggtgagc |
| 3570 | NM_004354 | CCNG2 | tttgggggagggttgttaaacagtaatagataagtgagacagattgcttgttatttatggtcaa |
| | | | atggtg |
| 3571 | NM_004356 | CD81 | tgtcctttctaacacgtcgccttcaactgtaatcacaacatcctgactccgtcatttaataaag |
| | | | aaggaa |
| 3572 | NM_004358 | CDC25B | tgattggagattactctaaggccttcctcctacagacagtagacggaaagcaccaagacct |
| | | | caagtacat |
| 3573 | NM_004422 | DVL2 | ttcggagacctcagtggtggctgtgagagctacctagtcaacctgtctctcaatgacaacg |
| | | | atggctcca |
| 3574 | NM_004445 | EPHB6 | tccgagaacttgcccgggaagtcgatcctgcttatatcaagattgaggaggtcattgggac |
| | | | aggctcttt |
| 3575 | NM_004510 | SP110 | acattgctgctcccatctttagaagcaatctaccatagttcaccctctggccacagtagttca |
| | | | catcttt |
| 3576 | NM_004529 | MLLT3 | acaaaagtgtacctgtaaatgtcctgaatccagcattgttgagctgtcatcaacattcttgtgt |
| | | | ctgttt |
| 3577 | NM_004556 | NFKBIE | ggcaggtgctcttgttttacccatgttgggtcagcctgaaactgccaaccagtaggaagcat |
| | | | ggactctc |
| 3578 | NM_004577 | PSPH | atcgatgagctagccaaaatctgtggcgttgaggacgcggtgtcagaaatgacacggcg |
| | | | agccatgggcg |
| 3579 | NM_004619 | TRAF5 | tcctgaagctgagcaagactgtccttttaagcactatggctgtgctgtaacggataaacgga |
| | | | ggaacctg |
| 3580 | NM_004635 | MAPKAPK3 | ggctgggaaaccctagaccatgtcagataggacaacactgctgggttttacatccagatag |
| | | | taataaaca |
| 3581 | NM_004737 | LARGE | ttgtccccgcgttcgagacactgcgctaccggctgtccttccccaagtcaaaagcggagtt |
| | | | gctgtcaat |
| 3582 | NM_004844 | SH3BP5 | tgttggatggcacagatgtagcacatgtcatcgtcacagaagttccgtcagccagcactgg |
| | | | agagcactt |
| 3583 | NM_004951 | EBI2 | ttgtttcgttctgggtcataaaactttgttaaggaactcttttggaataaagagcaggatgctg |
| | | | caaagt |
| 3584 | NM_005013 | NUCB2 | atttagatatgctaatcaaagcggcaacaagtgatctggaacactatgacaagactcgtcat |
| | | | gaagaatt |
| 3585 | NM_005077 | TLE1 | ccaagcggacagaggggccatgggttgtaggattgaggaacggaatctgccgactcaca |
| | | | tgacagcccat |
| 3586 | NM_005127 | CLECSF2 | catagcacttagacaggactttcatgacccaatatggaacaatttgagtaccagaatagga |
| | | | gattatggc |
| 3587 | NM_005211 | CSF1R | actcccccaagctgactcatcctaactaacagtcacgccgtgggatgtctctgtccacatta |
| | | | aactaaca |
| 3588 | NM_005213 | CSTA | ttgggtacatgctgctaaaagcccgtcagctcgtcatccttgtttttgtcaacctggtatccag |
| | | | taagta |
| 3589 | NM_005214 | CTLA4 | cagaacactgtcttgaagacaatggcttactccaggagacccacaggtatgaccttctagg |
| | | | aagctccag |
| 3590 | NM_005271 | GLUD1 | tatttggccagggccttttgaacagtagtgtccccatgaagtgctagataatatatgtgtaag |
| | | | agtcagc |
| 3591 | NM_005292 | GPR18 | aaaacaatttcaggctcgagtcattagtgtcatgctataccgtaattaccttcgaagcatgcg |
| | | | cagaaaa |
| 3592 | NM_005347 | HSPA5 | ctcaaaggctctcactaccgtggaccacctagtctgtaactctttctgaggagctgttactga |
| | | | atattaa |
| 3593 | NM_005348 | HSPCA | acttttcattccctgtagttgacaattctgcatgtactagtcctctagaaataggttaaactgaa |
| | | | gcaac |
| 3594 | NM_005415 | SLC20A1 | gaatgtgaacttcgggcaagttaaatgggacagccttccatgttcatttgtctacctcttaact |
| | | | gaataa |
| 3595 | NM_005449 | TOSO | acgcctttgccctgatccaaatgttagcacttgctagtgaacgtctacttatctcaagttctatg |
| | | | ctaaa |
| 3596 | NM_005461 | MAFB | ttcagtcccagctgtaggcttgtaaatacccgccccgccaaaccgcatagagaacgtggc |
| | | | agcaagctga |
| 3597 | NM_005531 | IFI16 | tgctttgaattggcaccgaaaagtgggaataccggggagttgagatctgtaattcatagtca |
| | | | catcaagg |
| 3598 | NM_005582 | LY64 | agcctgacatgcgacagcattgattctcttagccatcttaagggaatctacctcaatctggct |
| | | | gccaaca |
| 3599 | NM_005601 | NKG7 | gcgatatattctcgtccactcccccgctatccaacgtcacatctaagcactatgacatgcac |
| | | | cgaccgaa |
| 3600 | NM_005606 | LGMN | ttccggacccactgcttcaactggcactcccccacgtacgagtatgcgttgagacatttgta |
| | | | cgtgctgg |
| 3601 | NM_005610 | RBBP4 | atgcacaaatcgtcaaatctgaaaccatcttaacctcttccttagagtagctttctaattattct |
| | | | ccatt |
| 3602 | NM_005623 | CCL8 | aataggaaaattcctatccagaggctggagagctacacaagaatcaccaacatccaatgt |
| | | | cccaaggaag |
| 3603 | NM_005638 | SYBL1 | cacacctgaataaaattgatgtgcatgttttagggatcaattacctaactgttccttggtctattt |
| | | | atgt |
| 3604 | NM_005652 | TERF2 | tctgctgggctatgtaggcaggttaatcctccacttctcatgtggttgaaccagtgtgttttttg |
| | | | gtaaa |
| 3605 | NM_005658 | TRAF1 | gtcctagcaataacctcttgatccctactcaccgagtgttgagcccaaggggggatttgtag |
| | | | aacaagcc |
| 3606 | NM_005856 | RAMP3 | acttggaggaccccccagacgaggttctcatcccgctgatcgttatacccgtcgttctgact |
| | | | gtcgccat |
| 3607 | NM_005935 | MLLT2 | catcacaagaaaggtgaccgaagagctgacggagaccactgtgcttcggtgacagattc |
| | | | ggctccagaga |
| 3608 | NM_005950 | MT1G | cattatcaactagatactacacataccagcaccactactcacactacagcccactcaaaaa |
| | | | catcatcca |
| 3609 | NM_006006 | ZBTB16 | cctggatagtttgcggctgagaatgcacttactggctcattcagcgggtgccaaagcctttg |
| | | | tctgtgat |
| 3610 | NM_006014 | DXS9879E | accccaacgattacaacacaacaccagcgcccaatatacgcgaatgaggcgccctatac |
| | | | ccgcctctcca |
| 3611 | NM_006115 | PRAME | ccaggatgtgcatgcatcttgaagcaacaaagcagccacagtttcagacaaatgttcagtg |
| | | | tgagtgagg |
| 3612 | NM_006162 | NFATC1 | gtacaattagttgcttattacgtatgattactcacagcgatctattgttccatataaccaaaaag |
| | | | catgg |
| 3613 | NM_006164 | NFE2L2 | tgctcagttaggtcagacctggggccagttgcccatttagcatctcaggcctcaggatcctc |
| | | | atttataa |
| 3614 | NM_006195 | PBX3 | ccaatagaatttcaacatgttctgtagcttagagtgctcacttactacctctgaacaatactca |
| | | | cgctgt |
| 3615 | NM_006218 | PIK3CA | ggggatgatttacggcaagatatgctaacacttcaaattattcgtattatggaaaatatctgg |
| | | | caaaatc |
| 3616 | NM_006254 | PRKCD | cttttcctctgccttcggagggaaattgtaaatcctgtgtttcattacttgaatgtagttatctatt |
| | | | gaa |
| 3617 | NM_006257 | PRKCQ | cggatctcactcagccgcagacaagtaatcactaacccgttttattctattcctatctgtggat |
| | | | gtgtaa |
| 3618 | NM_006317 | BASP1 | gcagtacatcgtttgtacctgaaactgccgccacatgcactcctccaccgctgagagttga |
| | | | atagctttt |
| 3619 | NM_006332 | IFI30 | gagcaaaacttgatagctttgcatagggaaagagggcattgatgctggggttttgaaaggt |
| | | | gagtaggag |
| 3620 | NM_006475 | POSTN | gattcattacaattcaaatcgaagagttgtgaactgttatcccattgaaaagaccgagccttg |
| | | | tatgtat |
| 3621 | NM_006498 | LGALS2 | taatgccaccgccacgagggaactggcttacgctgttggcacatttccttccagacggttct |
| | | | ccacagat |
| 3622 | NM_006558 | KHDRBS3 | actgtgggaaaactcgagagcgctcaggaattgtaggtagcaattactgtaaaggtggatt |
| | | | tatggatta |
| 3623 | NM_006620 | HBS1L | ctgtgctgttacactaccaaactgtcagtgaacccgccgttattaaacgattgattagtgtctt |
| | | | aaacaa |
| 3624 | NM_006644 | HSPH1 | gatgaataatgtcatgaatgctcaggctaaaaagagtcttgatcaggatccagttgtacgtg |
| | | | ctcaggaa |
| 3625 | NM_006734 | HIVEP2 | atatgcaaagtcgatgagaatatgacccaaaggacactggtcaccaacgcagccatgca |
| | | | agggataggat |
| 3626 | NM_006763 | BTG2 | tcttgtctgcaaacaggtccctgcctttttagaagcagcctcatggtctcatgcttaatcttgtc |
| | | | tctct |
| 3627 | NM_006841 | SLC38A3 | aaaccaggagttcagctggctgcggcatgtgcttattgccgttggcctgctcacttgtatca |
| | | | acctgctg |
| 3628 | NM_006889 | CD86 | ccttgattagactcctagcacctggctagtttctaacatgttttgtgcagcacagtttttaataa |
| | | | atgct |
| 3629 | NM_006892 | DNMT3B | aagaagtcgtttttagggagaacgggaattcagacaagctgcatttcagaaatgctgtcata |
| | | | atggtttt |
| 3630 | NM_006986 | MAGED1 | aaagtgctgagattcattgcagaggttcagaaaagagaccctcgtgactggactgcacag |
| | | | ttcatggagg |
| 3631 | NM_007311 | BZRP | cccacccaagtgtctagcgggctcctcacactcaacatgtcctaaatgctgggtatcgcat |
| | | | gaagcactg |
| 3632 | NM_007360 | KLRK1 | ctccacaggaccaaaccatagaacaatttcactgcaaacatgcatgattctccaagacaaa |
| | | | agaagagag |
| 3633 | NM_007361 | NID2 | taactgcagtctacccctactgcccaacaggaagaaagtaagtacagtaatgtaaaggaa |
| | | | gacttggagt |
| 3634 | NM_012203 | GRHPR | gtgattctgatatatgtacttgtcacattggtgttggacacatttgcgccaaaagtatggtaatt |
| | | | ctatt |
| 3635 | NM_012448 | STAT5B | tctgttccagcaaccctggttggggggtcagacttgatacactttcaggttgggagtggacc |
| | | | caccccag |
| 3636 | NM_012452 | TNFRSF13B | tcgattcacccatcagcccatctccagcgctgtcccaattacagccctagcaagtggagac |
| | | | aacaagaag |
| 3637 | NM_013230 | CD24 | ccccgaagtcttttgttcgcatggtcacacactgatgcttagatgttccagtaatctaatatgg |
| | | | ccacag |
| 3638 | NM_014246 | CELSR1 | cttctgtgactccagtgactcagacgttagacctcttgatgttttcccactggtccctgaggct |
| | | | ctgttc |
| 3639 | NM_014257 | CD209L | cccagcttccagcggtactggaacagtggagaacccaacaatagcgggaatgaagactg |
| | | | tgcggaattta |
| 3640 | NM_014338 | PISD | tacaatgacttcagcttcgtgacgcacaccaatagagagggcgtccccatgcgtaagggc |
| | | | gagcacctgg |
| 3641 | NM_014456 | PDCD4 | ttgtaagtgccatgtttattatctaatcattccaagttttgcattgatgtctgactgccactccttt |
| | | | ctt |
| 3642 | NM_014686 | KIAA0355 | tctccatgggctgtgagactgtgtgaagccgtttgtgtggtctccatgtaggtgctgtgttcc |
| | | | cggcacc |
| 3643 | NM_014762 | DHCR24 | attgtcttagacactgtggttgcaaaacccacggaaagcctcatttgtgtggaaagtcagag |
| | | | gaaaaatg |
| 3644 | NM_015187 | KIAA0746 | tacatgctgctaaggtagtgaataaatcagtaatgcaatattgtgggtccaaactactctttgc |
| | | | actact |
| 3645 | NM_015641 | TES | ataaaagccaagccacccatttgcttttaatccaaagaacatgtatagtttttgtacccagag |
| | | | actatga |
| 3646 | NM_015866 | PRDM2 | ttccttatcctaatgatgcgcttttgtcccgtaaatgttaacactcatgaagcataccccggcc |
| | | | tctcag |
| 3647 | NM_016076 | PNAS-4 | atgtaaagggttctagtagaaatagtgtcctaaggccaattacctaccatactaacaatcag |
| | | | cagataaa |
| 3648 | NM_016081 | KIAA0992 | cttcccataaggagaccaaattgggtgaacacgcctcgaggagacctcaggataacagg |
| | | | tcaacacctgt |
| 3649 | NM_016187 | BIN2 | aaaagtatctatagacacagacacttgcctatacagagacataaccacacacactcagag |
| | | | gatagtgaac |
| 3650 | NM_016336 | UBE2J1 | cagcatcctttcatcaacctacccaacctgtagctaagaatacctccatgagccctcgaca |
| | | | gcgccgggc |
| 3651 | NM_016628 | WAC | accgttaagctgggcaaaggaaatgacaaggggacggggtctgtgagagtcaattcagg |
| | | | ggaaagataca |
| 3652 | NM_017530 | LOC55565 | ttagctccccggggtcattgggtcgccacctcttaatccactcggaggaccagcgatctaa |
| | | | ctgtgctgt |
| 3653 | NM_017784 | OSBPL10 | gaagctgttaaagatttatgccagagccttgcaggatggagcacctctgggacaactaag |
| | | | agccaaggcc |
| 3654 | NM_017935 | BANK1 | tgctttcagggtgaagcaagcttgaatttggattgcctgctttctttaaagcgaattcatactat |
| | | | aacag |
| 3655 | NM_017955 | CDCA4 | cctgggtgaaaatcaggaaccgcacacagccacatcttcctagacctaagagtaaattatg |
| | | | gaggatttt |
| 3656 | NM_018442 | IQWD1 | ggatactttgaacattagaaggccgctagtaaaaatggtttataaaggccatcgcaactcca |
| | | | ggacaatg |
| 3657 | NM_018842 | BAIAP2L1 | cttaagcaaatcatgcttctctgtttcacgtagttgggttgacaagtttctgcctttaagataaat |
| | | | gagt |
| 3658 | NM_020151 | STARD7 | tgaactacactgttcagataaggtcacaatctgatgctgtcagtttgaccgagctggttttgct |
| | | | tatggt |
| 3659 | NM_020199 | C5orf15 | aaaggatcccaaacttgtaactaaattctgacatatctgttactgctgactcacattcattctcc |
| | | | gccat |
| 3660 | NM_020529 | NFKBIA | acttggccccatgaacggtccatgccgataagtaaacgtcgaacaattgcccttcaaccac |
| | | | tccctctaa |
| 3661 | NM_021813 | BACH2 | accaagaatgtctataatgcatcatcacacagtacctcaggttttgcaagcacattccggga |
| | | | agataact |
| 3662 | NM_021822 | APOBEC3G | catagctgcttgtaggtgccacctcttaaggccattaccatagcaattaatactgaacatgag |
| | | | ctttgga |
| 3663 | NM_021950 | MS4A1 | gtggcacattcttagagttaccacaccccatgagggaagctctaaatagccaacacccatc |
| | | | tgttttttg |
| 3664 | NM_021966 | TCL1A | cgcctggctgcccttaaccatcgagataaaggataggttacagttacgggtgctcttgcgtc |
| | | | gggaagac |
| 3665 | NM_022552 | DNMT3A | ggaaagggtatttggtttcccagtccactatactgacgtctccaacatgagccgcttggcga |
| | | | ggcagaga |
| 3666 | NM_023037 | 13CDNA73 | cttaaacttaatgctgtcaagtgttagatgtgtgcatgtgaacttgttgcactgcagaaacata |
| | | | ttcaga |
| 3667 | NM_024708 | ASB7 | tcagtgataaaggtaccacaccgcttcagctcgccattatccgagagaggtcaagctgtgt |
| | | | gaaaatcct |
| 3668 | NM_025113 | C13orf18 | ctgtgccggctgtggaactccagtagagcctagtaagtacggataatggatcgcctgctta |
| | | | tgatgtgat |
| 3669 | NM_025216 | WNT10A | aggccacagatgtgagtaagcgagacacaacacttgtcctcttggaggttacattcttgctg |
| | | | gggggagg |
| 3670 | NM_030666 | SERPINB1 | aaactggaagagagttacactctcaactccgacctcgcccgcctaggtgtgcaggatctct |
| | | | ttaacagta |
| 3671 | NM_030764 | SPAP1 | cagacactgtgcaaataaattatttctgctaccttctcttaagcaatcagtgtgtaaagatttga |
| | | | gggaa |
| 3672 | NM_030775 | WNT5B | tcatccacgtgcacttgtgcggcatctgcagtttacaggaacggctccttccctaaaatgag |
| | | | aagtccaa |
| 3673 | NM_031305 | ARHGAP24 | taactctcccttcatatcttttcacctatttccagtccttatcatagttgataaaaacctcagact |
| | | | catc |
| 3674 | NM_031942 | CDCA7 | ccattcaatgtttgatgcataattggaccttgaatcgataagtgtaaatacagcttttgatctgt |
| | | | aatgc |
| 3675 | NM_032991 | CASP3 | gtatgacatttcacgggagatttcttgttgctcaaaaaatgagctcgcatttgtcaatgacagt |
| | | | ttcttt |
| 3676 | NM_033208 | TIGD7 | ctcctctcttcatattgtccgttttccggtattgagttagggaaagtatgcaggtattcctgtcat |
| | | | tcct |
| 3677 | NM_033274 | ADAM19 | ttccaaagtcagtaattgtgttaaccacgtgtataacagctctgctggacacccaagaaagc |
| | | | catgggaa |
| 3678 | NM_057735 | CCNE2 | cctaactggggctttcttgacatgtaggttgcttggtaataacctttttgtatatcacaatttggg |
| | | | tgaa |
| 3679 | NM_078467 | CDKN1A | cccctggaggcactgaagtgcttagtgtacttggagtattggggtctgaccccaaacacctt |
| | | | ccagctcc |
| 3680 | NM_079421 | CDKN2D | cagacgagacccaagggcagagcatttaagagtgaagtcatgacctccagggagccta |
| | | | gaagctggtggc |
| 3681 | NM_080593 | HIST1H2BK | acctcaaagctctcccgtggggcgattctcttgttgttccgatagtgtctcgcgggcgaaac |
| | | | caccagca |
| 3682 | NM_138340 | ABHD3 | ggataccaaacaattgatgattattatactgatgccagtccgagtcctagactgaagtcagt |
| | | | aggaattc |
| 3683 | NM_138379 | TIMD4 | tcataacccagcccccccggcaagttgatacttgatcatgaagtcccccaaaaataccaaa |
| | | | tacttttat |
| 3684 | NM_139207 | NAP1L1 | tgagtatgtcacttacccgtgcttctgtttactgtgtaattaaaatgggtagtactgtttacctaa |
| | | | ctac |
| 3685 | NM_139276 | STAT3 | gtaatttatataatccctgaaacgggcttcaggtcaaacccttaagacatctgaagctgcaa |
| | | | cctggcct |
| 3686 | NM_144646 | IGJ | ttaaccccagatgcctgctatcctgactaatttaagtcattgctgactgcatagctctttttcttg |
| | | | agag |
| 3687 | NM_145804 | ABTB2 | ctcctctgccctagagctgtttgttcaatcactgatgggcaaaggggagatgtagctaactg |
| | | | gtggccct |
| 3688 | NM_152785 | GCET2 | tactcttcagcagagttccctgatggccttgtacagagaacccgatgattgatgagggtata |
| | | | gcacagct |
| 3689 | NM_172373 | ELF1 | tgagccttgggctaatctggctgagtagtcagttataaaagcataattgctttatattttggatc |
| | | | atttt |
| 3690 | NM_175870 | LOC90925 | aggttgttcatggtgtatattacatggttaacatcaaaaggctgctaataggcacctcttcaat |
| | | | attaat |
| 3691 | NM_177968 | PPM1B | aatgcaggaggcagcgtgatgatacaacgtgttaatggttcattagcagtatctcgtgctct |
| | | | gggggact |
| 3692 | NM_181443 | BTBD3 | ctttatcccaccattagagagagaaatgcgatgatgttcaataatgatttgatggcagatgta |
| | | | cattttg |
| 3693 | NM_181838 | UBE2D2 | tttattgaacaagttagaggcataagcttaagagcatttccatgaaacaacacatgcagcatt |
| | | | ccaggaa |
| 3694 | NM_182746 | MCM4 | ctgaagcagtctgcaactgatccccggactggcatcgtggacatatctattcttactacggg |
| | | | gatgagtg |
| 3695 | NM_182776 | MCM7 | gagctcaatgtctggcaggtcaatgcttcccggacacggatcacttttgtctgattccagcct |
| | | | gcttgca |
| 3696 | NM_198833 | SERPINB8 | catacatggaaccatacatcatctatgctttgtagtatgactcctgtcactcagtacaattatttt |
| | | | gaga |
| 3697 | NM_201593 | CACNB2 | accgctgacatctcgcttgccaaacgctcggtattaaacaatcccagtaagcacgcaataa |
| | | | tagaaagat |
| 3698 | R99527 | MGC39372 | tgtggataaagaccccagataaagatgcctgtgttgctcaactcttgtaaatactcttgagac |
| | | | attttga |
| 3699 | T66903 | LOC54103 | atacagtggcataaacgatgctcttcctagtagcttaataggccacaagctagtttctgttgc |
| | | | actctga |
| 3700 | U37028 | ITGAD | tggacccaggtcgtctgacttctcgtgccattttcaatgaaaccaagaaccccactttgactc |
| | | | gaagaaa |
| 3701 | W56129 | SFRS9 | gtctttgtgggttgctctactatggagatcaacagttactgtgactgagtcggcccattctgttt |
| | | | agaaa |
| 3702 | X58529 | IGHM | cacccatagcatgttaaccctgccgcgtggaatccccccgggaatcgaaattccgaaccc |
| | | | aatgcagtcc |
| 3703 | AA188785 | LOC286025 | gtaacatactcatcaactatgatagacttaacacttgttacctaatgaacaaggaggatgtgc |
| | | | atttcgg |
| 3704 | AA601122 | SECTM1 | aaaatctgaaagaaacgtctttagtggctttaagccccaaaacgtccctaaggcgtcctcg |
| | | | agatgaaga |
| 3705 | AA642467 | SLC2A14 | agtgaaacctgaggattcctcgttgaagcaggtattcatccatggggttcttccgcggtgaa |
| | | | gccagctt |
| 3706 | AA682722 | LARP | gcttcacacaacacgtctaccataagtatcgtaggcgctgccttaatgagcggaaacgctt |
| | | | gggcattgg |
| 3707 | AA757235 | LOC399933 | ctaactgtttattgttggtatgtagaaatatatctgatttgcatactggtcctttatccagcaacc |
| | | | ttct |
| 3708 | AA765313 | FZD3 | tttgcatgactgatagctgtaactcacagttaacatgctttcagtcaagtacagattgtgtcca |
| | | | ctggaa |
| 3709 | AB002310 | HUWE1 | gcagagactcaccgcactgtgttaaaccagatcctacggcagtccacgacccaccttgct |
| | | | gatgggcctt |
| 3710 | AB002333 | ZNF518 | catttcagaatagccatatcaggaaaataatgtcgagatatgtacttcttgcctttaagttttaa |
| | | | cagcc |
| 3711 | AB024402 | ING1 | caggaaattactgcccacttgtcaagttcagcccaccatctgtttgaagattatatgaagttta |
| | | | aattct |
| 3712 | AB028952 | SYNPO | ttggctggtagagccttctagaggttcagaatattagcttcaggatcagctgggggtatgga |
| | | | attggctg |
| 3713 | AB032991 | NDFIP2 | tcatcctgtgtataattgtagactgtaccaagaagcaactaccttagctccactgccctttga |
| | | | gggatgg |
| 3714 | AF008915 | EVI5 | aggtatagtggatgctatttatgatccaagtttttacatcacatccattgatctgcctatcccttc |
| | | | tttt |
| 3715 | AF108138 | C15orf20 | gaacataagctgcaaaatgtgcacccatattttaagctgctttttcaggggataaatagtgttt |
| | | | ggggac |
| 3716 | AF116668 | LMO2 | agggtggggtaagggatttcttaggggatggtagacctttattgggtatcaagacatagcat |
| | | | ccaagtgg |
| 3717 | AF143327 | FLJ32810 | gtggatctattcaaagcttaacttctgtaggttccaaggagacacccaaagcttcaccaaac |
| | | | ccagacct |
| 3718 | AF200348 | D2S448 | ggatcccaccctaccacgactacagggtctactgcaatctatcggcggcacacacgttcg |
| | | | aggacctgaa |
| 3719 | AF245480 | MONDOA | tctttgtattggagatatttctgtaactcattctcttggtgctcacgattgccatggccataggg |
| | | | ccaca |
| 3720 | AI075159 | SPTA1 | gcacaactgattgatgagcggacaaagcttggagactatgccaacctaaaacaattctacc |
| | | | gagaccttg |
| 3721 | BU948323 | TCF4 | ttcatcttttgcatccaacaggcagaatatgatctgtgtccaaaagtgaacttgagtcaggaa |
| | | | tgaatca |
| 3722 | AI675029 | C7 | acagatacaaactatttctatcctgagtagtaatctcacacttcatcctatagagtcaaccacc |
| | | | acagat |
| 3723 | AI694012 | PSMA5 | ggggaaagtgagtcaatacctgttagtgttaggctattaacaatagttaggtgaaaggtaat |
| | | | gagggcat |
| 3724 | AI732248 | COL4A1 | gacctgcaattactacgcaaacgcttacagcttttggctcgccaccatagagaggagcga |
| | | | gatgttcaag |
| 3725 | AJ007509 | HNRPUL1 | tgaacttgaaaccttttattccgggcgtcttggtagtttctggtgggattcagtgggtgagag |
| | | | ggaagaa |
| 3726 | AK022224 | ACTN1 | gctgtattcatttctccaatctcatgtccattttggtgtgggagtcggggtagggggtactctt |
| | | | gtcaaa |
| 3727 | AK023928 | PSCD1 | agacagagaggcacctgggtcagtattagtctatttatcagaggtgtaaataatctatgtata |
| | | | gtttttc |
| 3728 | AK024580 | NRP1 | tacagaaaacggtgccatccctgaacacggctggccactgggtatactgctgacaaccgc |
| | | | aacaacaaaa |
| 3729 | AK025005 | MARCH1 | ttataaaccagagaaaggcagcaaatatgtctatctttttaaatattcatgataatggaccag |
| | | | gcacggt |
| 3730 | AK091853 | COL3A1 | atggagcaaaacagtctttgaatatcgaacacgcaaggctgtgagactacctattgtagata |
| | | | ttgcaccc |
| 3731 | AK126650 | CAPZB | acatagggacctacacacagaggagtgaacttagggttctagggactatggccgggtca |
| | | | ccggtggccag |
| 3732 | AK127124 | ARHGEF4 | aaagatgccggaagccccaaaaagcccaccctagtgagtctgcctctaggacccgaagt |
| | | | tctctccccag |
| 3733 | AK127132 | DRG1 | gaagcctttactagcagcttcaacccagtcctccttaccttgcacctattaacgttggttcaac |
| | | | atgaag |
| 3734 | AK129940 | GNAI1 | ctttagttttcgacattatttttaggttttaagagtggcaacttaggattttagggtgatggctttg |
| | | | gaa |
| 3735 | AL049313 | CLIC5 | ttggactggtggcatcatgtaactgctaaccttacagtaaaaacccaagaatgggtcaaaa |
| | | | atgtcttcc |
| 3736 | AL049327 | CBLB | cagagtcagcatgctaattcactctagttcaaatccacattctagttgaatccctgcaatgtaa |
| | | | cttgac |
| 3737 | AL049449 | GAB1 | gatcaggtttagacaatgagctttggttgtgttcttgttagtcctaatattggttttcagtttggaa |
| | | | tta |
| 3738 | AL050391 | CASP4 | caagaagaatcatgatgagggatgggatgaaggcagcaaggggtaaccatcttgacctt |
| | | | ctgttttttct |
| 3739 | AL137527 | LRCH3 | ccctgcggtattgttttccctctgttgtatgtatagatatatgctacttgtcaattttccaattttga |
| | | | aa |
| 3740 | AL353942 | SEPT11 | gacctgtagtggcatgagagtgtataacatttcctaagatgacaggcaaagtagaactaac |
| | | | cctggcact |
| 3741 | AL833915 | TDRD9 | ccagagagagttgcgcagcttcaagacattgcccgtcagaagcttttaggtttgttctgtca |
| | | | gtcaaaac |
| 3742 | AL834278 | USP30 | atgtgaccttaaagtgtactgcaaagctgtaactttaagtaattgctgttctgccactgcttact |
| | | | ctgaa |
| 3743 | AU252485 | MAP7 | gcggtcagtttccaccatgaatctttcgaaatatgttgatcccgtcattagcaagcggctctc |
| | | | ctcttca |
| 3744 | AV650179 | PRG1 | acagctcgtccttgggagagaaaaagctcccacagtatgtggcgtctgaggggtaacag |
| | | | cattatatttc |
| 3745 | AV686223 | KIAA1407 | tcccacctgcaactgctgaataatacactcagtttgtcttctagtactcagctcagtttgatcta |
| | | | gatat |
| 3746 | AW014009 | MAP2K3 | gagcaccttgcagacgtgatcttgcttcgtcctgcagcactgtgcggggcaggaaaatcc |
| | | | aagaggaaga |
| 3747 | AW190768 | SLC13A3 | tcattactctccacccccttactctgctataatttctccttaataggacactgtataacttattttct |
| | | | ta |
| 3748 | AW292143 | GFAP | ggttgtgggtgaccagttgcacttggcctctggattgtgggaattaaggaagtgattcatcct |
| | | | cttgaag |
| 3749 | AW439398 | CCND1 | tggctgaagtcacctcttggttacagtagcgtagcgtggccgtgtgcatgtcctttgcgcct |
| | | | gtgaccac |
| 3750 | AW452039 | HRMT1L3 | cattttcttagacgtttgctccaccagatttaaccaaatgtaactcccacattgagtttatctata |
| | | | ttga |
| 3751 | AW615076 | LOC492304 | gtccattggacctaaactacacgcagactgagcatagacagagcgcagacaatcacaaa |
| | | | gcagaaaagca |
| 3752 | AW664012 | ALOX5 | caagtgcaaagttcacacctgatttttaagacttagaataaggaggtggggaatgtgaaata |
| | | | tctcattt |
| 3753 | AW967479 | CD38 | aaggaaaaacaaataagcgagaaccgcgacgagcaccacataagccgtaggaccgca |
| | | | tgaaagaagagac |
| 3754 | AW970654 | PHTF2 | ttcaaagagaattgtggcagatgttgtgtgtgaactgttgtttctttgccacatgtgttgtatttg |
| | | | aaag |
| 3755 | BC028066 | NALP1 | accaggaaccaccttggcaagatatttacccaccggccatctctgtttactcatgaatgttaa |
| | | | atgttaa |
| 3756 | BC034289 | LOC87769 | gggagagctcacttactcttcaacttaatggcagactgtgatcgctaactgcatctggcaca |
| | | | aaaacatg |
| 3757 | BC042366 | PCDH9 | tcatcatcaccgcaactctgacgtttcctacaagaagttagagacttaagcagtattggcatc |
| | | | ggatgga |
| 3758 | BC045532 | LSM8 | gaattttacaagctggttaattgatgtcttgttagtagcttgaaattggctgtggtgggagtgtt |
| | | | tgcac |
| 3759 | BC050383 | TMPO | gacacagtatcatgaaagtcctatttcagtaagacccatttacatacagtagatttttagcag |
| | | | agatctt |
| 3760 | BC050602 | HIST1H2AC | gctggaatggtggtaagtgaactgaaaattccagtcactcttgggctagactcaacgttctt |
| | | | aaaaacta |
| 3761 | BC058855 | VEGF | gggaaaagatattaacatcacgtctttgtctctagtgcagtttttcgagatattccgtagtacat |
| | | | attta |
| 3762 | BE147267 | ECE1 | tctgggaacaccaagcaccctgaatcgagactgcaggagccctgcggggtgagactgtg |
| | | | tcagagataca |
| 3763 | BE549606 | BZW1 | tattcaaggcttaactgaaaccggtactgatttggaagcagtagctaagtttcttgatgcttct |
| | | | ggagca |
| 3764 | BE798965 | LAMC1 | gtggcatcctacttacgtacgtggcataacacatcgtgtgagcccatgtatgctggggtag |
| | | | agcaagtag |
| 3765 | BE927772 | SFRS3 | caaaattacactatccacctggtacctaagtcttgtcatggacttacaggtttgcatgggttcc |
| | | | aaatac |
| 3766 | BF055235 | RIPK1 | gggcctgggtgagttctgtttgctccttgtaccaccatccaaatggtgttatcaaatctcttag |
| | | | attcca |
| 3767 | BF513638 | WNT3 | ggggctttctttttagttctctagggtctgataggaacagacctgaggcttatctttgcacatgt |
| | | | taaag |
| 3768 | BG682263 | HDAC3 | tgtggagatttaagagtggcttgggatgctgtgtcccaaggaatttcttttcacctcttggaag |
| | | | ggctgg |
| 3769 | BG683220 | PMS2 | tacatctcaggtttcatttcacaatgcacgcatggagttggaaggagttcaacagacagaca |
| | | | gtttttct |
| 3770 | BI493513 | PHF8 | gggaggggttgggagtgttagtaatcaaggtttagaacaccatgagatagttacccctgat |
| | | | ctccagtcc |
| 3771 | BI559738 | GPR56 | actggaaccgacatgctgggagattacatcttctctatggcaagcgtgacttcttgctgagt |
| | | | gacaaagc |
| 3772 | BM709325 | BCL7A | ttccacaaagtgctggcacttacacccacaacccggaaggctgtggaccgattcctctag |
| | | | ggtggtgacc |
| 3773 | BM711190 | MED28 | caggactttgatctcttctgttgccctcgctaggttctccctggtgggttagtgctttgtatctgt |
| | | | ctct |
| 3774 | BM727177 | GOLT1B | aagcattattagacttgaaagggtttgataatctcccagtccttagtaaagattgagagaggc |
| | | | tggagca |
| 3775 | BQ009245 | GPM6B | caactatctaaaacctaattgcaaattgaccacagacctctaacctcctacttttatagacttg |
| | | | aatact |
| 3776 | BQ021469 | AGPS | aaggatttgacccaaatcagctaagtgtagccacattactgtttgagggggatcgtgagaa |
| | | | ggttcttca |
| 3777 | BQ215285 | LEF1 | gcttcttgtcacctaacaaaggcgtaggagtaacctcgcactttgctagctaacctgactcc |
| | | | gggtgagg |
| 3778 | BQ898221 | ITGA6 | agactgtagctcagtattcgggagtaccttggtggatcatcctagtggctattctcgctggga |
| | | | tcttgat |
| 3779 | BU929213 | SESN3 | tgactggactaatgtaaggcagatgacgtgatctttaagactgctatatatatcagtctcttac |
| | | | tctata |
| 3780 | BU954396 | RPL11 | cagatgtgaattctcaaaagttagccattgctgcaatctctgctgttgcctcctgttctgaaaa |
| | | | aattaa |
| 3781 | BX095432 | CNTN5 | gatgtaagccttaagccttcgcagtttccacgtggcattgagcgttcgagtgcacagaacta |
| | | | gaagtcaa |
| 3782 | BX640908 | EVI1 | cccctttccgaatttcaaggaccaaggtgacccgacctgtgtatgagagtgccaaatggtg |
| | | | tttggcttt |
| 3783 | BX648339 | USP53 | caaggaaaaggatcatataaacatgaccgagttgtacctcagagtcgagcttctgcacaaa |
| | | | taataagtt |
| 3784 | BX648365 | SEPT7 | tggcactgctaaaatagaatatagcatctttcatatggtaggaaccaacaaggaaactttcct |
| | | | ttaactc |
| 3785 | BX649193 | TKT | tcaccatcaagcccctggacagaaaactcattctcgacagcgctcgtgccaccaagggc |
| | | | aggatcctcac |
| 3786 | C05886 | ETV6 | gtacctgcctaatccacccggcgtgactcatttcaacactaagtactaggggtgttgtcagg |
| | | | agacaaat |
| 3787 | D31762 | TRAM2 | cacttacatcatagataaattttagcttcccagtaactgagggatttgtttcctaacgccattgg |
| | | | aggcc |
| 3788 | D86967 | EDEM1 | gtgctggtgaaaaggtagggctgagtgattaccttagccacagggtggctgagcaggaa |
| | | | ctttagaagaa |
| 3789 | H41942 | NPEPPS | ggaatggttaaacacaaaaggctgatagctggtatcacatagttggagtcagtgcataattc |
| | | | caagtggc |
| 3790 | M60028 | HLA-DQB1 | aagaaggctttgcggatccccggagaccttcgggtagcaactgtcaccttgatgctggcg |
| | | | atgctgagct |
| 3791 | M77810 | GATA2 | gggaggtagctaattctcctctgtagctcttgcaatcccgttgattctaacatcaggcttctga |
| | | | gagttc |
| 3792 | N26272 | MSI2 | tcctcgttttgaatataagattcccaaacagaggagcataacacattcctagtacctaagttc |
| | | | caggtgg |
| 3793 | N28431 | COL6A1 | atgccatggactttatcaacgacgccaccgacgtcaacgatgccctgggctatgtgaccc |
| | | | gcttctaccg |
| 3794 | N41620 | WWP1 | gggaagtcatctgtaactaaaggtggtccacaaattgcttatgaacgcggctttaggtgga |
| | | | agcttgctc |
| 3795 | N53163 | COL27A1 | gtcgagtttgccatcagccgggtccagatgaatttcctgcacctgctaagctccgaggtga |
| | | | cccagcaca |
| 3796 | N54946 | EWSR1 | atagacgtgtcatgtagtggggtgtaatcggtatatgaacggtagggtagatggtattagatt |
| | | | ctaattt |
| 3797 | N70000 | UBE3B | cttttgtaactggtgttcactgacaccttgatggctcttgatggctctaaaaagttgtaggatttt |
| | | | ttgt |
| 3798 | NM_000016 | ACADM | caaaatctatcagatttatgaaggtacttcacaaattcaaagacttattgtagcccgtgaaca |
| | | | cattgac |
| 3799 | NM_000024 | ADRB2 | cctacacccttggacttgaggattttgagtatctcggacctttcagctgtgaacatggactctt |
| | | | ccccca |
| 3800 | NM_000033 | ABCD1 | agcctaatttattggattccctattcgtagccatctccgtggccaatgtgactaccgtgccag |
| | | | cagcggg |
| 3801 | NM_000062 | SERPING1 | atgctctcaatcatggagaaattggaattcttcgatttttcttatgaccttaacctgtgtgggct |
| | | | gacag |
| 3802 | NM_000089 | COL1A2 | tccttgatattgcacctttggacatcggtggtgctgaccatgaattctttgtggacattggccc |
| | | | agtctg |
| 3803 | NM_000099 | CST3 | aaacacacctctgaggccagcggccagcgtctgactcacaagccatgactgccccacct |
| | | | tcttacaacaa |
| 3804 | NM_000110 | DPYD | tattgtcgactgcatcaaaatggtttccaggacaacaccttatgaaccaaagagaggcgta |
| | | | cccttatct |
| 3805 | NM_000118 | ENG | agtagccaagctgcttgtcctgggcctgcccctgtgtattcaccaccaataaatcagaccat |
| | | | gaaacctg |
| 3806 | NM_000120 | EPHX1 | ctccaggcttttcttggggaagataccccttttctgaggaatgagtttgcctccgtcccctgc |
| | | | ccatgct |
| 3807 | NM_000147 | FUCA1 | attaaaggaactgagtacattaccctgatgtctaaatggaccaaagatctgagatccattgt |
| | | | gattatat |
| 3808 | NM_000169 | GLA | tgggctgtagctatgataaaccggcaggagattggtggacctcgctcttataccatcgcag |
| | | | ttgcttccc |
| 3809 | NM_000182 | HADHA | ctgtctgtgcagatgctttgcccaggcttctcaccacggtgtaccgggatattaaacctcttt |
| | | | ccccagc |
| 3810 | NM_000194 | HPRT1 | gaaaagtaagaagcagtcaattttcacatcaaagacagcatctaagaagttttgttctgtcct |
| | | | ggaatta |
| 3811 | NM_000214 | JAG1 | gtgtcggtcttccagtctccagccggtgaagacaaagtgcacctctgactcctattaccag |
| | | | gataactgt |
| 3812 | NM_000234 | LIG1 | agtgacaagggcatctcccttcgcttccctcggtttattcgagtccgtgaagacaagcagc |
| | | | cggagcagg |
| 3813 | NM_000246 | MHC2TA | tcaattctcatttgtcacacactttggctattagagatcaaccccttcgctcctgtgtcttgcaa |
| | | | tggca |
| 3814 | NM_000249 | MLH1 | gtgccccctttggagggactgcctatcttcattcttcgactagccactgaggtgaattggga |
| | | | cgaagaaa |
| 3815 | NM_000251 | MSH2 | ttgggattcatgttgcagagcttgctaatttccctaagcatgtaatagagtgtgctaaacaga |
| | | | aagccct |
| 3816 | NM_000277 | PAH | actttgctgccacaatacctcggcccttctcagttcgctacgacccatacacccaaaggatt |
| | | | gaggtctt |
| 3817 | NM_000305 | PON2 | ctcagtgtatgatgggaagctgctcataggcactttataccacagagccttgtattgtgaact |
| | | | ctaaatt |
| 3818 | NM_000341 | SLC3A1 | ataatatgatttcgggccttcccgctaaaataagaataaggttaagtaccaattctgccgaca |
| | | | aaggcag |
| 3819 | NM_000361 | THBD | caaagctttttccagaggctattattttcactgtagaatgatttcatgctatctctgtgtgcacaa |
| | | | atat |
| 3820 | NM_000382 | ALDH3A2 | tggtctggacaagaagcgagtaagccactgcggttggtcatactgaagggaattgatggc |
| | | | aagaggatcc |
| 3821 | NM_000395 | CSF2RB | ggagtccagagcccacgtctactgcggaaaagtcaggggaaactgccaaacaaaggaa |
| | | | aatgccccaaag |
| 3822 | NM_000417 | IL2RA | caaatcaatgcgtacgtttcctgagaagtgtctaaaaacaccaaaaagggatccgtacatt |
| | | | caatgttta |
| 3823 | NM_000418 | IL4R | ctcaggtgctagggaaggcgggaaccttgggttgagtaatgctcgtctgtgtgttttagtttc |
| | | | atcacct |
| 3824 | NM_000435 | NOTCH3 | acacaggcatgactgggatcccgtgtaccgagtacacgacccaggtatgtaccaagtag |
| | | | gcacccttggg |
| 3825 | NM_000448 | RAG1 | gaggcaggatatataagcgccagtggtagttgggaggaataaaccattatttggatgcag |
| | | | gtggtttttg |
| 3826 | NM_000484 | APP | caagaagagtagaagacagagccttactttataatcagaattatgaaaagaggagccaat |
| | | | gagagggaaa |
| 3827 | NM_000485 | APRT | ggaagcagctccgggtaggtgatgggtaacattccttaaatggtgcatgtcactggcctttc |
| | | | agctggga |
| 3828 | NM_000536 | RAG2 | ttatataccttcaagcaaccctttggattatgcccatgaacaagttagtttctcatagctttaca |
| | | | gatgt |
| 3829 | NM_000546 | TP53 | ttggtagtttctacagttgggcagctggttaggtagagggagttgtcaagtcttgctggccca |
| | | | gccaaac |
| 3830 | NM_000561 | GSTM1 | tctgcccatataagaagaactgtgcggcacagcgcccgctactacagctcgtcacacggt |
| | | | gttgtccgac |
| 3831 | NM_000574 | DAF | gtcttctatctgggcacacgtgtttcacgttgacaggtttgcttgggacgctagtaaccatgg |
| | | | gcttgct |
| 3832 | NM_000578 | SLC11A1 | atcgagctagccattgtgggctccgacatgcaggaagtcatcggcacggccattgcattc |
| | | | aatctgctct |
| 3833 | NM_000594 | TNF | atatttatctgattaagttgtctaaacaatgctgatttggtgaccaactgtcactcattgctgag |
| | | | cctct |
| 3834 | NM_000598 | IGFBP3 | taggctttgtgttatgtgatggcactatttcaattggctttctaattggacaattgatactatgct |
| | | | atct |
| 3835 | NM_000601 | HGF | tatcttgtgccaaaacgaaacaattgcgagttgtaaatgggattccaacacgaacaaacat |
| | | | aggatggat |
| 3836 | NM_000604 | FGFR1 | acctctgccccagataggtggtgccagtggcttattaattccgatactagtttgctttgctgac |
| | | | caaatg |
| 3837 | NM_000610 | CD44 | accaagaattgattttgtagccaacattcattcaatactgttatatcagaggagtaggagaga |
| | | | ggaaaca |
| 3838 | NM_000628 | IL10RB | gtaattttcagccttttatatcactaaaataagatcatgttttaattgtgagaaacagggccga |
| | | | gcacag |
| 3839 | NM_000629 | IFNAR1 | aatgaagtttgctgtggtattgcatagccttgctttcttgaactaaactgtttgcccttcacaag |
| | | | tagtt |
| 3840 | NM_000640 | IL13RA2 | tgatacatgaagactttccatatcaagagacatggtattgactcaacagtttccagtcatggc |
| | | | caaatgt |
| 3841 | NM_000660 | TGFB1 | tgctggcacccagcgactcgccagagtggttatcttttgatgtcaccggagttgtgcggca |
| | | | gtggttgag |
| 3842 | NM_000700 | ANXA1 | tcagaagatgtatggtatctccctttgccaagccatcctggatgaaaccaaaggagattatg |
| | | | agaaaatc |
| 3843 | NM_000701 | ATP1A1 | gagtttggaactctaccctggtaggaaagcaccgcagcatgtggggaagcaagacgtcc |
| | | | tggaatgaagc |
| 3844 | NM_000717 | CA4 | caccacaccgacctgcgatgagaaggtcgtctggactgtgttccgggagcccattcagct |
| | | | tcacagagaa |
| 3845 | NM_000732 | CD3D | agtttcagatcctcgtaggccacaggggaaaaaggtccatttgtctcaccgggaggctca |
| | | | atattgcctg |
| 3846 | NM_000773 | CYP2E1 | agtgcagagcgcttgtacacaatggacggtatcaccgtgactgtggccgacctgttctttg |
| | | | cggggacag |
| 3847 | NM_000801 | FKBP1A | ggcgggggcaccatattgcgcacaaacacccctatcacgaaaattacaccccccaacgg |
| | | | caacttttaca |
| 3848 | NM_000852 | GSTP1 | ttgggtcggccagttcagcggatgctatggtgccggggcgacaaggtcgcaactataatc |
| | | | taaccgcctt |
| 3849 | NM_000856 | GUCY1A3 | tgactttgactcaataaaccactcaccctattattggaaagtgccataggccttgagaaagta |
| | | | tagttac |
| 3850 | NM_000867 | HTR2B | actgaagagcgagttagttatgtatagcagaactggcagttgtcatcaaacataatgatgag |
| | | | taagatga |
| 3851 | NM_000878 | IL2RB | cccctaccccgacccctccacgtatctttcctaggcagatcacgttgcaatggctcaaaca |
| | | | acattccac |
| 3852 | NM_000885 | ITGA4 | cactggcaatagtatggctcccaatgttagtgtggaaataatggtaccaaattcttttagccc |
| | | | ccaaact |
| 3853 | NM_000887 | ITGAX | cagcatcagctcagggcttcatcgtggggctctcagttccgatttcccaggctgaattggg |
| | | | agtgagatg |
| 3854 | NM_000905 | NPY | tgtgctgcactcctgcaatgttaaccttggtcagcactcggcacacccctgcgcggtgcac |
| | | | ataacagca |
| 3855 | NM_000906 | NPR1 | cttactgggaggagaaagagtcacctgaaggggaacatgaaaagagactaggtgaaga |
| | | | gagggcagggga |
| 3856 | NM_000953 | PTGDR | aaacgcagctgcaactgaagcggagactctaaacccagcttgcaggtaagagctttcacc |
| | | | tttggtaaaa |
| 3857 | NM_000960 | PTGIR | gaaggggaggctgctgcattgtgggtgatgacgtaggacatgtgcttggtacaaaaaggg |
| | | | cctgagacat |
| 3858 | NM_000964 | RARA | ggcaaatacactacgaacaacagctcagaacaacgtgtctctctggacattgacctctggg |
| | | | acaagttca |
| 3859 | NM_000967 | RPL3 | ggctcagagtgagcgctgggtacagcgcccgaatcggacagtgtagaaccattctctact |
| | | | gccttccttc |
| 3860 | NM_000968 | RPL4 | gaagggctctttggatctctgggcttttcaagattctgctaagatctgtattaatcatcttgtgc |
| | | | atggg |
| 3861 | NM_000970 | RPL6 | gggcacgaggggacatatctgtgaaccctccacggtggaaaaaatgccgcccttagtcg |
| | | | ggtggggaaaa |
| 3862 | NM_000972 | RPL7A | aaccttgaaaatctcagaaaacagtaagccaagctaactgcctctttttgtcttttcagatccg |
| | | | ccgtca |
| 3863 | NM_001001547 | CD36 | tttgatagttctgaagagcaaatgaatcctagtacattgaagagtaccgtactctatctggca |
| | | | cttaatt |
| 3864 | NM_001005 | RPS3 | atggaagagggtgtagtaatgcgtatcagaggtagcggagaatgaagagcgacaagtgg |
| | | | gacgagctgta |
| 3865 | NM_001009 | RPS5 | gaagagcgaggagcgaagaaggaagacgagacgactagaagcacacaataagacga |
| | | | caaccagacacaaa |
| 3866 | NM_001015 | RPS11 | acaatgccgggggccctcagcttggtgttggagaaaggcccatagtgtcatgttacagcat |
| | | | cctgtgggg |
| 3867 | NM_001019 | RPS15A | tgagtgttgatgtttttctgcactagaaggcactatgttgaactattaaacttaccagcactttct |
| | | | tttt |
| 3868 | NM_001069 | TUBB2 | gtgcggaggaagtaattaggttgggcgagtcggtatcggttgtgatgttggcgtatcgtatg |
| | | | tctggagg |
| 3869 | NM_001079 | ZAP70 | tcctgaagcgcgataacctcctcatagctgacattgaacttggctgcggcaactttggctca |
| | | | gtgcgcca |
| 3870 | NM_001092 | ABR | atacagccttttgggtaaacactgggaactccagaagttgtggggagagtggggaatcag |
| | | | acagccgcct |
| 3871 | NM_001130 | AES | ccacacctgtgtctgtatatagtcagcttatctcgtgttcaatcgtctgatctctacagagaga |
| | | | agtgga |
| 3872 | NM_001150 | ANPEP | gtacctgagctacaccctgaacccggacttaatccggaagcaggacgccacctctaccat |
| | | | catcagcatt |
| 3873 | NM_001165 | BIRC3 | tctagtagtatgcaaagattgtgctccttctttaagaaagtgtcctatttgtaggagtacaatca |
| | | | agggt |
| 3874 | NM_001175 | ARHGDIB | attagcaacacttagccaactatatgccacctatctgcctatgtatacttttcaatgcccaact |
| | | | attctt |
| 3875 | NM_001178 | ARNTL | ctggaagctgtaggctaatgggactggcccacagctttcctactctacttcattaccacact |
| | | | gggtttca |
| 3876 | NM_001207 | BTF3 | attgtgtgtcattgtattctttggttctgctcccccactattgaccaatgtatgagatgggaaga |
| | | | ggggg |
| 3877 | NM_001211 | BUB1B | tctggaaacttagccaaaatatttctgagctaaaagatggtgaattgtggaataaattctttgt |
| | | | gcggat |
| 3878 | NM_001219 | CALU | agggaagccgtgcttctgaggaacaactctaattagtacacttgtgtttgtagatttacacttt |
| | | | gtatta |
| 3879 | NM_001226 | CASP6 | ttcctttggatgtagtagataatcagacagagaagttggacaccaacataactgaggtggat |
| | | | gcagcctc |
| 3880 | NM_001228 | CASP8 | gcaataaataccagacacgtacaaaatccagctatgaatatagagggcttatgattcagatt |
| | | | gttatcta |
| 3881 | NM_001229 | CASP9 | ggatttttggctacacacaaaccagtatcgctcatagatcagcaaaccggggcctactaga |
| | | | gtctgaaca |
| 3882 | NM_001237 | CCNA2 | ataccctggaaagtcttaagccttgtctcatggaccttcaccagacctacctcaaagcacca |
| | | | cagcatgc |
| 3883 | NM_001239 | CCNH | accaatgtcattctgccacgtatgttccatatgtcctatctatctgccctgcacaattatgaga |
| | | | gggaag |
| 3884 | NM_001255 | CDC20 | gcaaagaggcaagcgcaagacgacaggaaacgaagcgagagacgagaagccggtg |
| | | | acgagcaggagaaag |
| 3885 | NM_001256 | CDC27 | gagccccactattacatctcccccaaacgcactgcctcgaagaagttcacgactctttacta |
| | | | gtgacagc |
| 3886 | NM_001265 | CDX2 | taacatccaagccagagtttttaaagccttctggatccatggggggagaagtgatatggtg |
| | | | aagggaagt |
| 3887 | NM_001273 | CHD4 | tgaggttacagatgtcagagcgtaacattctcagccgcctggcaaaccgggcacccgaa |
| | | | cctaccccaca |
| 3888 | NM_001274 | CHEK1 | tttcaaccttcggtgtgcttgggagtggcgattgtgatttacacgacaaaatgccgaggtgct |
| | | | cggtgga |
| 3889 | NM_001280 | CIRBP | agtccttgcccacaacgatgtggagccctgtgaaagtcggattcgaataaagggccacgt |
| | | | gtgcacccag |
| 3890 | NM_001295 | CCR1 | cattacctacacttccactacccaaagacaaaatgtgcccactgtgtgcttttgagtgtatttt |
| | | | ctttta |
| 3891 | NM_001313 | CRMP1 | gaaacctccaccagtccaacttcagcttatcaggtgcccagatagatgacaacaatccca |
| | | | ggcgcaccgg |
| 3892 | NM_001337 | CX3CR1 | gagagaataagtcatgttccttcaagatcatgtaccccaatttacttgccattactcaattgat |
| | | | aaacat |
| 3893 | NM_001344 | DAD1 | gatgaagagtggcggcagacagagcgcaggagagcgaacagaacgaccaagaagag |
| | | | ccagacgaagagca |
| 3894 | NM_001345 | DGKA | tgagcaacctgtccctagaaggcatcgcagtgctaaacatccctagcatgcatggtggctc |
| | | | caacctctg |
| 3895 | NM_001398 | ECH1 | aaggagatgatgtggcccggatcagctggtacctccgtgacatcatcactcgataccagg |
| | | | agaccttcaa |
| 3896 | NM_001404 | EEF1G | cccctaatcactgtcgcctgtaattctccaaaacccttccaccaattcatgcgctatcaaatgt |
| | | | attatt |
| 3897 | NM_001428 | ENO1 | ttctcgcctcactttccaccaagtgtctagagtcatgtgagcctcgtgtcatctccggggtgg |
| | | | ccacagg |
| 3898 | NM_001436 | FBL | gagcaagcacataaacgagggaacacatcatacaccagtccgcgaagcgctaggacca |
| | | | tacgagacagac |
| 3899 | NM_001441 | FAAH | gtagtggtgcatgtgctgaagctgcagggtgccgtgcccttcgtgcacaccaatgttccac |
| | | | agtccatgt |
| 3900 | NM_001456 | FLNA | aagaaccgcaccttctccgtctggtacgtccccgaggtgacggggactcataaggttact |
| | | | gtgctctttg |
| 3901 | NM_001459 | FLT3LG | agactgtcgctgggtccaagatgcaaggcttgctggagcgcgtgaacacggagatacac |
| | | | tttgtcaccaa |
| 3902 | NM_001462 | FPRL1 | agagtcaaatttagagcagaaaatactagcaaaggggactgaaaagcggtggccaattg |
| | | | agcttcaaatg |
| 3903 | NM_001489 | NR6A1 | tgatgattttggcccttgatggatctaaaactcggaacattactcctcaatgccgtttccccag |
| | | | ttgctc |
| 3904 | NM_001513 | GSTZ1 | ctggccttgaaaggcatcgactacgagacggtgcccatcaatctcataaaggatgggggc |
| | | | caacagtttt |
| 3905 | NM_001515 | GTF2H2 | tgaatgtaaaatctgtggtcttactttggtgtctgctccccacttggcacggtcttaccatcatt |
| | | | tgttt |
| 3906 | NM_001540 | HSPB1 | ttgtcggtcaggcgcgacaccacgctcgcaatgataccacacatcactacaactccgccc |
| | | | gacgacaaac |
| 3907 | NM_001560 | IL13RA1 | catcccctgtgggaaattagtaggctcatttactgttttaggtctagcctatgtggattttttcct |
| | | | aaca |
| 3908 | NM_001607 | ACAA1 | taagggccagccttgaaagggatactagcctgcagtgggcatctgtcaccgtgtgtgtgcc |
| | | | caagtggga |
| 3909 | NM_001630 | ANXA8 | taacaaaataaactacaaattcctgacccaaggacactgtgttataagaggcgtgggctcc |
| | | | cctggtggc |
| 3910 | NM_001640 | APEH | ccctcaagacccggaatgtgcctgttcggctcctgctctatcccaaaagcacccacgcatt |
| | | | atcagaggt |
| 3911 | NM_001654 | ARAF | gctcattgtcgaggtccttgaagatgtcccgctgaccatgcacaattttgtacggaagacctt |
| | | | cttcagc |
| 3912 | NM_001664 | RHOA | aaaggaccctggtgggccagacgggttggacatcgttaataatcatagttggcttctaaata |
| | | | ctggtagc |
| 3913 | NM_001712 | CEACAM1 | ctttctccaggaggattagcttggagttctctatactcaggtacctctttcagggttttctaacc |
| | | | ctgac |
| 3914 | NM_001715 | BLK | ccagcttgtcgcaagtaacgacacggaacagctcgaacgctcgtgactttattgcagaaa |
| | | | agccccgcag |
| 3915 | NM_001716 | BLR1 | cctcgcaagctgggtaatcgatgggggagtctgaagcagatgcaaagaggcaagaggc |
| | | | tggattttgaat |
| 3916 | NM_001728 | BSG | tggaacccggcacagtcttcactaccgtagaagaccttggctccaagatactcctcacctg |
| | | | ctccttgaa |
| 3917 | NM_001734 | C1S | actagggagggtgtgaggggcacggtgccatgtggggatgttggtgatatggccctgtgt |
| | | | gttatacctt |
| 3918 | NM_001743 | CALM2 | gttgcactattgcaaaacgggtgtattatccaggtactcgtacactatttttttgtactgctggt |
| | | | cctgt |
| 3919 | NM_001747 | CAPG | ccgacaacgaagcacgcacaaggaacgcaccagatcaaccgacagcagaacgcacg |
| | | | agagccaagaccat |
| 3920 | NM_001752 | CAT | ggcttcaaaatagagaatcccactttctatagcagattgtgtaacaattttaatgctatttcccc |
| | | | agggg |
| 3921 | NM_001753 | CAV1 | tcaccggggtggtttagtggctcaacattgtgttcccatttcagctgatcagtgggcctccaa |
| | | | ggagggg |
| 3922 | NM_001759 | CCND2 | atgatcagtgtatgcgaaaaggtttttaggaagtatggcaaaaatgttgtattggctatgatg |
| | | | gtgacat |
| 3923 | NM_001767 | CD2 | tttgactctacactcaaagccatctgatgctcacatttcatgtagtcagaggaggccatctgg |
| | | | tttgaat |
| 3924 | NM_001772 | CD33 | caaaatctggtacattcctgcaatgcatatcattcagacataaagatgggccgaagtacag |
| | | | atacatgct |
| 3925 | NM_001774 | CD37 | agctggagcgaagcttgcgggacgtcgtagagaaaaccatccaaaagtacggcaccaa |
| | | | ccccgaggagac |
| 3926 | NM_001776 | ENTPD1 | ccactgcagtttaatggatgagatgcccacttttaagccaaaaatactaagctcagactgga |
| | | | cctcctct |
| 3927 | NM_001783 | CD79A | ccagagaaacgaagtgagcagaactcgtaggaaaggctcgtgggtaaggagagtgcgc |
| | | | gcggtgactgca |
| 3928 | NM_001786 | CDC2 | ttcttatgccttggtcagagtaataactgaaggagttgcttatcttggctttcgagtctgagttt |
| | | | aaaac |
| 3929 | NM_001793 | CDH3 | aatgcctcccattcggatggatctctgcgtttttatactgagtgtgcctaggttgccccttatttt |
| | | | ttat |
| 3930 | NM_001794 | CDH4 | gcactgccaaagcttgtccgtgggagtgccacactcataactgccaggttccgacttctgt |
| | | | ccaccctag |
| 3931 | NM_001795 | CDH5 | gaccgaggttcccactctgggcaaagcccctcacactgcaagggattgtagataacactg |
| | | | acttgtttgt |
| 3932 | NM_001798 | CDK2 | cttccttttagtattcttagttgttcagttgccaaggatccctgatcccattttcctctgacgtcca |
| | | | cct |
| 3933 | NM_001830 | CLCN4 | ggaaaatattgagctaggcacattactctctgaacgaaattcatattatcttattaaggaaga |
| | | | gtgttgg |
| 3934 | NM_001838 | CCR7 | atcaaagccacactctgggctccagagtggggatgacatgcactcagctcttggctccact |
| | | | gggatggga |
| 3935 | NM_001839 | CNN3 | gtgtatctggtttctacatggaagactaaactcatgcttattgctaaatgtggtctttgccaact |
| | | | aaatt |
| 3936 | NM_001897 | CSPG4 | ttgaggccaacatgttcagcgtcatcatccccatgtgcctggtacttctgctcctggcgctca |
| | | | tcctgcc |
| 3937 | NM_001901 | CTGF | gtttttcggacagtttatttgttgagagtgtgaccaaaagttacatgtttgcacctttctagttga |
| | | | aaat |
| 3938 | NM_001905 | CTPS | gccatgaaaccagagacaaaatctctatactgccctgagttggggggaattctcagtgcca |
| | | | actgtggct |
| 3939 | NM_001908 | CTSB | gcagagaatcacgtcctggaactgcatgttcttgcgactcttgggacttcatcttaacttctc |
| | | | gctgccc |
| 3940 | NM_001916 | CYC1 | gggggccgaacttgaacctcaacacgacagccaacgtgcataagcatgcaacactaccg |
| | | | aggtagccaca |
| 3941 | NM_001923 | DDB1 | caagatgcaggaggtggtggcaaacctacagtatgacgatggcagcggtatgaagcga |
| | | | gaggccactgca |
| 3942 | NM_001928 | DF | caccgctatggacagagcgcagtagtgattagtcgtatttcgcgacacctcacacggtaga |
| | | | tgtgatcaa |
| 3943 | NM_001939 | DRP2 | aggtgcatgcgggcagtgatgggagaaggggaggcatgattcttctgaccctagaaatgt |
| | | | tccctttaat |
| 3944 | NM_001941 | DSC3 | tttgctcactcccactcaccgatcaaaacctgctacctccccaagactttactagtgccgata |
| | | | aactttc |
| 3945 | NM_001952 | E2F6 | gcagccttgctgttgagtcagtccaaggggttttacttaggacaagttgtaccttgccctctct |
| | | | ccagct |
| 3946 | NM_001964 | EGR1 | tttggcttataaacacattgaatgcgctttattgcccatgggatatgtggtgtatatccttccaa |
| | | | aaaat |
| 3947 | NM_001993 | F3 | tttgcacataacatgctttagattatatattccgcacttaaggattaaccaggtcgtccaagca |
| | | | aaaaca |
| 3948 | NM_001999 | FBN2 | cctgatcccactgctgttgaacagatcagcctagagagtgtcgacatggacagccccgtc |
| | | | aacatgaagt |
| 3949 | NM_002015 | FOXO1A | catgcctcctgcatttcgctacccgagtttagtaacagtgcagattccacgttcttgttccgat |
| | | | actctg |
| 3950 | NM_002017 | FLI1 | atgatgacatgtcgaagtctacccaatatcccccagtaagagaataaggggcaagaatga |
| | | | ctcatgaact |
| 3951 | NM_002023 | FMOD | tatagtttggctggggagcacttaattcttcccatttcaaaaggtaatgttgcctggggcttaa |
| | | | cccacc |
| 3952 | NM_002033 | FUT4 | gtaacctcttcaactggacgctctcctaccgggcggactcggacgtctttgtgccttatggc |
| | | | tacctcta |
| 3953 | NM_002046 | GAPD | acctcttttgccccccacacaaaagcccctttggtgtgaaaaacggcccccacaagtttata |
| | | | aaaggggg |
| 3954 | NM_002049 | GATA1 | tcatccggcccaagaagcgcctgattgtcagtaaacgggcaggtactcagtgcaccaact |
| | | | gccagacgac |
| 3955 | NM_002064 | GLRX | aacatctgataaaacttacagccccctacaccaagagtgtatctgtgaaagagctcctaca |
| | | | ctttgaaaa |
| 3956 | NM_002065 | GLUL | gccatgtacagtatgttcaaagtcaaataactcctcattgtaaacaaactgtgtaactgccca |
| | | | aagcagc |
| 3957 | NM_002068 | GNA15 | atggaatctgcagccgctccatggcctcgatcatggcccgcatggacacgaagatgttct |
| | | | ggtagaccag |
| 3958 | NM_002076 | GNS | atctcagtcatttgacttagaaagtgcccttcaaaaggaccctgttcactgctgcacttttcaa |
| | | | tgaatt |
| 3959 | NM_002086 | GRB2 | ctgcttctcttttcctcgcagatgataaaaggaatctggcattctacacctggaccatttgatt |
| | | | gtttta |
| 3960 | NM_002090 | CXCL3 | gaccttacattcacactttggatgttcttgaggtgaattccctgcagtgtctgcaagcactgg |
| | | | cagcgca |
| 3961 | NM_002094 | GSPT1 | atcagctaagctcccttggcgatctgcaggaacactagtaatgactggaattactccgtgat |
| | | | ctttgatg |
| 3962 | NM_002102 | GYPE | agtttcacaacatcctgtaacattggcttacctgtatattatatgccacccgtaaccataaaaa |
| | | | caccag |
| 3963 | NM_002107 | H3F3A | aggaggcaaaaccaaaagtttatgtggtgcttggggtggaccggtcggcggatgtcgtgc |
| | | | gttaattaaa |
| 3964 | NM_002108 | HAL | ctgagaggtgtcggatgctcttggctttaaggatcaatgtcttagccaaaggatacagtggc |
| | | | atttccct |
| 3965 | NM_002110 | HCK | caccaggaatcagggaggcaggctctgaggacatcatcgtggttgccctgtatgattacg |
| | | | aggccattca |
| 3966 | NM_002118 | HLA-DMB | aattattgtccaaggctatatttctgggatgaatataatctgaggaagggagttaaagaccct |
| | | | cctgggg |
| 3967 | NM_002121 | HLA-DPB1 | ggagcaagaaagttcaacgaggatctgcataaacagggttcctgacctcaccgaaaaga |
| | | | ctaatgtgcct |
| 3968 | NM_002128 | HMGB1 | gtcaaatttcctaactttgctgtgcaaaggttgagagctattgctgattagttaccacagttctg |
| | | | atgat |
| 3969 | NM_002129 | HMGB2 | aaaatgcaggttgtagctttttgatgggctactcatacagttagattttacagcttctgatgttg |
| | | | aatgt |
| 3970 | NM_002135 | NR4A1 | aggtttggcacccacttctccacaccttgagggctcggggatactggatacacccgtgacc |
| | | | tcaaccaag |
| 3971 | NM_002141 | HOXA4 | cttgcatttatttaagacactgttacagagatactgttgtcaccttctggggcacggtctttgg |
| | | | ggagag |
| 3972 | NM_002145 | HOXB2 | aaaatattgggcactcggttatcttttaaaattcacacagaaaaattccgtttggtagactcctt |
| | | | ccaat |
| 3973 | NM_002162 | ICAM3 | atttgcctcgtgagctggacgcttggcactgataagtaccattatgtgttacgttgacgaaga |
| | | | acgggat |
| 3974 | NM_002166 | ID2 | tacatcttggacctgcagatcgccctggactcgcatcccactattgtcagcctgcatcacca |
| | | | gagacccg |
| 3975 | NM_002167 | ID3 | aaggtcttttcagagcgtggaggtgtggaaggagtggctgctctccaaactatgccaagg |
| | | | cggcggcaga |
| 3976 | NM_002186 | IL9R | cagatgtgggcggcctgtgtgaacctgtcgtgcaaagctcacgtcaccaactgctgcagtt |
| | | | atctcctga |
| 3977 | NM_002193 | INHBB | cctctagatgtccaagtgccacgtgaactatgcaatttaaagggttgacccacactagacg |
| | | | aaactggac |
| 3978 | NM_002198 | IRF1 | agtggcagccaacaaacgtggatgggaaggggtacctactcaatgaacctggagtccag |
| | | | cccacctctgt |
| 3979 | NM_002199 | IRF2 | ttgcggttactctttaggccttgtctgtaaaacgaatggctcaggctaagtgatctctaaatgc |
| | | | ctaaat |
| 3980 | NM_002222 | ITPR1 | acccccgtgaaatatgcccgcctctggtcggagattccctcggagatcgccattgacgact |
| | | | atgatagta |
| 3981 | NM_002224 | ITPR3 | agttgtacgcccaggcaacaagggctgagctgcgcttgcgtggctgtttcatgaccgcttg |
| | | | tttttctcc |
| 3982 | NM_002228 | JUN | ttcaaagccgggtagcgcgcgcgagtcgacaagtaagagtgcgggaggcatcttaatta |
| | | | accctgcgctc |
| 3983 | NM_002229 | JUNB | actaaaatggaacagcccttctaccacgacgactcatacacagctacgggatacggccg |
| | | | ggcccctggtg |
| 3984 | NM_002230 | JUP | atagtagttaccttgcagggtcgttgtcaggttatgtgagaaaaaataagcagggcattgaa |
| | | | ttcactgc |
| 3985 | NM_002248 | KCNN1 | gtgggaatcagaaaaacctgttcccatcaccggcctagcctagaatcctagcctagaagc |
| | | | cctctctccc |
| 3986 | NM_002276 | KRT19 | aggggatgcgaaggacggggaccttaaccccgggtcttctcctgagctgccgaataaac |
| | | | ttttggtccca |
| 3987 | NM_002312 | LIG4 | ctagtgaggaaagcctctgatctggcagactcattgcagcaggtggtaatgataaaatact |
| | | | aaactacat |
| 3988 | NM_002341 | LTB | ccacatgaatggtagatgtaggcagcatgagtggttaagagtcaagggagtagccgggg |
| | | | tagtggcttac |
| 3989 | NM_002349 | LY75 | aacattttcccaagtgtgggaacattatttttagattgagtaggtaccttgtagcagtgtgcttt |
| | | | gcatt |
| 3990 | NM_002351 | SH2D1A | aagtacatgataagcgaggttccccgtgtgtaggtagatctggtctttagaggcagatagat |
| | | | aggtcagt |
| 3991 | NM_002353 | TACSTD2 | agaggagaggcgacagtcgcgagccacactttgcaatgaaactctttagactttctgccg |
| | | | ggagagcggc |
| 3992 | NM_002356 | MARCKS | ttaaactcaaccaagctgtgataagtggaatggttactgtttatactgtggtatgtttttgattac |
| | | | agca |
| 3993 | NM_002379 | MATN1 | agcgagccgcaggacgaacacgtcgattacgtggagagctacagcgtcatcgagaagc |
| | | | tgtccaggaagt |
| 3994 | NM_002388 | MCM3 | aggagtcggccacctacattgcagaagagtattcacgcctgcgcagccaggatagcatg |
| | | | agctcagacac |
| 3995 | NM_002390 | ADAM11 | ctaggttatgggtacggcaaccacatgtcccagatcgtctccaattcgaaaacaaccgtcc |
| | | | tgctgtccc |
| 3996 | NM_002392 | MDM2 | aaatgcacttcatgcaatgaaatgaatcccccccttccatcacattgcaacagatgttgggc |
| | | | ccttcgtg |
| 3997 | NM_002393 | MDM4 | acagaagtaatctgattagtcaaattattaagtgccatggattactttatgcagcagtcaggta |
| | | | catagt |
| 3998 | NM_002398 | MEIS1 | agtctatgttttagagctgcttaaaacaggaagtgatgtataaggtggtggttgttgcatggg |
| | | | gacaatg |
| 3999 | NM_002412 | MGMT | ggcgtggaggcaccgctgtattaaaggaagtggcagtgtcctgggaacaagcgtgtctg |
| | | | ccctttctgtt |
| 4000 | NM_002414 | CD99 | gggtgttctggtcgccgccccggatggtggtttcgatttatccgatgcccttcctgacaatga |
| | | | aaacaag |
| 4001 | NM_002415 | MIF | tctgtgcctctgcgcgggtctcctggtccttctgccatcatgccgatgttcatcgtaaacacc |
| | | | aacgtgc |
| 4002 | NM_002417 | MKI67 | tgcttccttctacgtaggccaacctcaaaactttcagtaggaatgttgctatgatcaagttgtt |
| | | | ctaaca |
| 4003 | NM_002419 | MAP3K11 | aggaaggctggaagcgcgagatccagggtctcttcgacgagctgcgagccaaggaaaa |
| | | | ggaactactgag |
| 4004 | NM_002421 | MMP1 | gaacacatgtgcagtcactggtgtcaccctggataggcaagggataactcttctaacacaa |
| | | | aataagtgt |
| 4005 | NM_002422 | MMP3 | tttatgttattacagggcattcaaatgggctgctgcttagcttgcaccttgtcacatagagtga |
| | | | tctttc |
| 4006 | NM_002436 | MPP1 | ttggagaaagcgtttttaactgcaactttctataatgccaaaatgacacatctgtgcaataga |
| | | | atgatgt |
| 4007 | NM_002441 | MSH5 | cttggtgagaattttctaacagatttacactcagagcctggtcgccccccgttagcccagatt |
| | | | caaaagg |
| 4008 | NM_002444 | MSN | aggctgagtagaagatcctaccccaattccttgtaggagtataggccggtctaaagtgagc |
| | | | tctatgggc |
| 4009 | NM_002447 | MST1R | agcacctcgcatgagatgaatgtgcgtccagaacagccgcagttctcacccatgccagg |
| | | | gaatgtacgcc |
| 4010 | NM_002460 | IRF4 | tgtaagttatgttttacatgccccgtttttgagactgatctcgatgcaggtggatctccttgaga |
| | | | tcctg |
| 4011 | NM_002462 | MX1 | tctctgcttatccgttagccgtggtgatttagcaggaagctgtgagagcagtttggtttctagc |
| | | | atgaag |
| 4012 | NM_002466 | MYBL2 | tgtctctgaccaactcccagttctctacagagcacgggaaagcccctcggggacgtctttc |
| | | | ctgcagtgt |
| 4013 | NM_002475 | MLC1SA | gtttccgcgtacgaacacctaaggatctactaaccgttcgcattctacttaacgcaggtcctt |
| | | | tctcttg |
| 4014 | NM_002483 | CEACAM6 | acacaggagattccagtctacttgagttagcataatacagaagtcccctctactttaactttta |
| | | | caaaaa |
| 4015 | NM_002512 | NME2 | taggtgatcacacacaccagccctgaacacgtgcggacaacgacacgcttcgcgcgctg |
| | | | ttggatgtaac |
| 4016 | NM_002514 | NOV | tggcatctgcacggcggtagagggagataactgtgtgttcgatggggtcatctaccgcagt |
| | | | ggagagaaa |
| 4017 | NM_002527 | NTF3 | ctctcaggagtcactctgtaaaatctgtgtacaccagtattttgcattcagtattgtcaaggcc |
| | | | atgact |
| 4018 | NM_002530 | NTRK3 | acggataactttatcttgtttgacgaagtgagtcccacacctcctatcactgtgacccacaaa |
| | | | ccagaag |
| 4019 | NM_002572 | PAFAH1B2 | gctacttgaactgtcaggggcatctgcctaaaccagaatcttttgtcagaaaccttaaccca |
| | | | acaaaaca |
| 4020 | NM_002576 | PAK1 | catgtcagccccattgactaccttctcatgctctgaggtactactgcctctgcagcacaaatt |
| | | | tctattt |
| 4021 | NM_002585 | PBX1 | tagaattttagaggcatagaagagagatcctaggtctagagactacaatccaaatagacta |
| | | | agttgggaa |
| 4022 | NM_002592 | PCNA | tcagacgtgtgaacggcacaccgacaatcacatggacaccccccaagaagtgatacgag |
| | | | ccaagccccca |
| 4023 | NM_002600 | PDE4B | agaaaggagcagtggtgtcgttcaccgtgagagtctgcatagaactcagcagtgtgccct |
| | | | gctgtgtctt |
| 4024 | NM_002608 | PDGFB | tggagtgataatattgtttccctcgtccgtctgtctcgatgcctgattcggacggccaatggt |
| | | | gcttccc |
| 4025 | NM_002609 | PDGFRB | agttggtccaaggagggagagtgggttctcaatacggtaccaaagatataatcacctaggt |
| | | | ttacaaata |
| 4026 | NM_002616 | PER1 | atctccaggagtccatgagaggctttcttctcctatgtcccaattctcagaactcagatgtgg |
| | | | ctagacc |
| 4027 | NM_002621 | PFC | agggtatatatttcggaggtaaaagaaagtagctcatgccaacaagtaacattcaccagca |
| | | | tttattgag |
| 4028 | NM_002648 | PIM1 | ctgttttcttcatcatgagttctgctgaatgccgcgatgggtcaggtaggggggaaacaggt |
| | | | tgggatgg |
| 4029 | NM_002658 | PLAU | attaggaagtgtaacagctgaggtctcttgagggagcttagccaatgtgggagcagcggtt |
| | | | tggggagca |
| 4030 | NM_002659 | PLAUR | ctgctaatgactgccagactgtggggaggcactctcctctggacctaaacctgaaatcccc |
| | | | ctctctgcc |
| 4031 | NM_002687 | PNN | caaaggataagaattcccggtccgacagaaagaggtctatatcagagagtagtcgatcag |
| | | | gcaaaagatc |
| 4032 | NM_002704 | PPBP | aacttctttaactcaccaggaagggtagaattttgaaacctgattttctatacgttctcatttatt |
| | | | cagg |
| 4033 | NM_002729 | HHEX | ttaaggggatagtacctcccaattcaagcagagaaactgacctgactaaagttaatcgcag |
| | | | atgaactag |
| 4034 | NM_002730 | PRKACA | acagggtctgccggggaagtttcaacattagccaggcggtgatgttcaccttgggatacca |
| | | | tggggccga |
| 4035 | NM_002744 | PRKCZ | gaaaaacgcccacgtcagggtgaccatctgtggtaaccgagaattccttggaggcagcgt |
| | | | cttcaccgcc |
| 4036 | NM_002753 | MAPK10 | ggtccatttcatgtgatctattactctgacataaacccatctgtaatatattgccagtatataag |
| | | | ctgtt |
| 4037 | NM_002758 | MAP2K6 | gctttcctttgagattggatagcaaaccatataagtagtattccttatcatggctgagaacata |
| | | | agaaga |
| 4038 | NM_002791 | PSMA6 | gatgtcaccctgtgctctcgtcacagactaacctgtcgtaggttaacagatcgcgagcaca |
| | | | cacgtgctg |
| 4039 | NM_002800 | PSMB9 | ataatatcaggcagttaccatacgagaaatcaaggtcgttgggacggaagtaaccttatct |
| | | | gcttttccc |
| 4040 | NM_002814 | PSMD10 | ttgtgataggagggatatttacatattttagtggaccacattttaagttggatggtgtgctctaa |
| | | | aatac |
| 4041 | NM_002827 | PTPN1 | aaaggcacaagatcacatttccagttaccctattacattgatatatacggccatgaaaagta |
| | | | gatggatt |
| 4042 | NM_002831 | PTPN6 | tgaccaactgcggggagcatgacacaaccgaatacaaactccgtaccttacaggtctccc |
| | | | cgctggacaa |
| 4043 | NM_002835 | PTPN12 | tagctgataaaatctctaagccacaggaattaagttcagatctaaatgtcggtgatacttccc |
| | | | agaattc |
| 4044 | NM_002844 | PTPRK | tttgtaattgactaaagcattccagtgtcctctttctagattgccagctcatgacatggtgcttat |
| | | | aaag |
| 4045 | NM_002856 | PVRL2 | ggtgtcacggtcacctgcaaagtggagcatgagagcttcgaggaaccagccctgatacct |
| | | | gtgaccctct |
| 4046 | NM_002868 | RAB5B | tcttcaaaggcccagtttcccctcacgcagcctcttaggtagcgtttcccctaatcgtgggg |
| | | | gttggacc |
| 4047 | NM_002870 | RAB13 | cttcaccccttatagttggctcgagagcatggaatccgatttttcgaaactagtgctaaatcc |
| | | | agtatga |
| 4048 | NM_002880 | RAF1 | ctttctccagaggcagaacacatgttttcagagaagctgctgctaaggaccttctagactgc |
| | | | tcacaggg |
| 4049 | NM_002887 | RARS | ggatgatggcagaaagattgtatttgtcccagggtgttccataccattaaccatagtaaaatc |
| | | | agatgga |
| 4050 | NM_002893 | RBBP7 | agagtatgttacatggcatataacggattgattaccttagatgtttgcacctaactgtatatga |
| | | | gcaaat |
| 4051 | NM_002901 | RCN1 | cttttcgactttatactctgagttattacttactgtaagtggtgtatatgaaacctccatgcatttt |
| | | | cca |
| 4052 | NM_002908 | REL | ggcagtatgcaaaatgagcaattgagtgactcctttccatatgaattttttcaagtataacttg |
| | | | caagat |
| 4053 | NM_002913 | RFC1 | cagaccagtaggaaccaggctgggtcaggctcttaatttcactatggtgggggacaaaat |
| | | | gaacactggc |
| 4054 | NM_002934 | RNASE2 | acccaaatatgacctgtcctagtaacaaaactcgcaaaaattgtcaccacagtggaagcca |
| | | | ggtgccttt |
| 4055 | NM_002935 | RNASE3 | agaggggcctgtgggttgagacactatagagtgtgtcataaccgagaccggatagggga |
| | | | gtagttacttc |
| 4056 | NM_002936 | RNASEH1 | aaaatggtggtattctcattctgtcgttttgcttgcctttattagctggaatggttttctaagaaa |
| | | | gtgt |
| 4057 | NM_002953 | RPS6KA1 | gagcatctggctgtttagcagaactcattctatccccaatcagctccttttccgttctgttctgc |
| | | | tggga |
| 4058 | NM_002960 | S100A3 | cgtccgaaacacccgaactggtcaactctcaagagaccatctggttcaggttcctgactgg |
| | | | gccagcgag |
| 4059 | NM_002961 | S100A4 | ctgtatacgatcgcatacaatcatgtctcacagcgctgatcttagcacattatgtcatgtttcc |
| | | | atctca |
| 4060 | NM_002971 | SATB1 | ggaagaggaccccaagactgcatcccagtctttgctggtaaaccttcgggctatgcagaat |
| | | | ttcttgcag |
| 4061 | NM_002972 | SBF1 | atgtggaccggctgagcaagaggacgcctgtgttccacaattacatgtatgcgcccgagg |
| | | | acgcagaggt |
| 4062 | NM_002975 | CLEC11A | cgaacgggtgtgctgatctcgtggacatgcttccgctccagctgttagcgacttgagctcgt |
| | | | cttcacag |
| 4063 | NM_002983 | CCL3 | tccaagcccggtgtcatgtaagtgccagtcttcctgctcacctctatggaggtagggaggg |
| | | | tcagggttg |
| 4064 | NM_002984 | CCL4 | tcgaattcataatatcgatggagcgggggagggagataataaaagaatagaagggttcctt |
| | | | cccccccac |
| 4065 | NM_002985 | CCL5 | ctgcatcaacacttcagattctacattaccactcaaacgcgaaacacaatcaaaatacccg |
| | | | agagaccgc |
| 4066 | NM_003002 | SDHD | ccggataaacaggtgtctaacgggcaatcgcctagaaacgatagagacagaacgcatgc |
| | | | gcgcaggcggg |
| 4067 | NM_003005 | SELP | ccttgccaacctgtgaggctatttcgtgtgagccgctggagagtcctgtccacggaagcat |
| | | | ggattgctc |
| 4068 | NM_003009 | SEPW1 | cgtttgcaagcgtatcacggatgtcacatcacactactcggatgacaatttcaacccgcaa |
| | | | aagaacaac |
| 4069 | NM_003025 | SH3GL1 | ccgaaagtcctggaccggtggctagcaatatcgtgtgttgcgcactcgtaaagtgtgttattt |
| | | | caaccct |
| 4070 | NM_003038 | SLC1A4 | agtccacagagtcggtagaccaggggttacgtgactggggaaaatctcacatctccttgtc |
| | | | tgaaaacat |
| 4071 | NM_003039 | SLC2A5 | tctgaaggacacggggacgttctcaccagcacatggtattaccggcctcgtcggtgtggtc |
| | | | tgtctgatg |
| 4072 | NM_003058 | SLC22A2 | caggaatacaacttaagtaatcatgaggtccttccatatttagattgggtaagcatgaatgtg |
| | | | tattttc |
| 4073 | NM_003072 | SMARCA4 | tggatgcatgtgcgtcaccgtccactcctcctactgtattttattggacaggtcagactcgcc |
| | | | gggggcc |
| 4074 | NM_003073 | SMARCB1 | acatgatcacggatacacgactctagccaccagtgtgaccctgttaaaagcctcggaagt |
| | | | ggaagagatt |
| 4075 | NM_003100 | SNX2 | tttatctagagctttgtctcagcttgcagaggttgaggagaagatagaccagttacatcaag |
| | | | aacaagct |
| 4076 | NM_003113 | SP100 | ccactaatctgtgactgctcctgtggaaactccacatcacaattctccaaaatttatcattgcc |
| | | | atttta |
| 4077 | NM_003118 | SPARC | cataatcctgcaagtactaggcgtggatatctggataatgaaggagtgtgaattaagaagg |
| | | | agtaccagg |
| 4078 | NM_003121 | SPIB | ccacaaagactgagttctcatggggatcctacccttcctagtgccactccctatggccatgc |
| | | | tgaagacc |
| 4079 | NM_003123 | SPN | ctcttcttgacgcttttcataatgtgcaaagtcatcaaagattgaggtggtatgcttgaaagta |
| | | | gcaatt |
| 4080 | NM_003127 | SPTAN1 | agcacgccaacgccttccaccagtggatccaagagaccaggacatacctcctcgatggg |
| | | | tcctgtatggt |
| 4081 | NM_003128 | SPTBN1 | tgaactgggacaggactatgagcatgtcacgatgttacaagaacgattccgggagtttgcc |
| | | | cgagacacc |
| 4082 | NM_003133 | SRP9 | caaggtgttaggtgtgactgtcacaactgttatgttttccagtaaactagaagtacgatatttg |
| | | | ataatt |
| 4083 | NM_003159 | CDKL5 | ctttcagctgcccaacccagcaatccgggttctctttcttcgtgagacacgttatgagggaa |
| | | | gccctgat |
| 4084 | NM_003189 | TAL1 | agcatttgggtaatttagattgtaaaccgctttggcctgagttattgagattgtcctcatttctcc |
| | | | agat |
| 4085 | NM_003200 | TCF3 | accctctttcaggtggattcagagacctgtcctgtatataacagcactgtagcaataaacgt |
| | | | gacatttt |
| 4086 | NM_003222 | TFAP2C | cttggaagattctatttaattgaaactctctgttcagaaagcaataactttgtctcgttcctgttg |
| | | | ggct |
| 4087 | NM_003225 | TFF1 | aaagagatcgatattataccaacctgtgcggcccaccgcgaagtcgtactccgactcggc |
| | | | cgccccaatc |
| 4088 | NM_003234 | TFRC | catcttcaaagcttgaagatcgttagtatctaacatgtatcccaactcctataattccctatcttt |
| | | | tagt |
| 4089 | NM_003262 | TLOC1 | tgaatcccttagaggtctgtgaattgtaggttaagaacccctgccctagctaatcagatcca |
| | | | ccaatgtt |
| 4090 | NM_003288 | TPD52L2 | cacttggagttgttttggttgcaggtgacagaaagcacgactcaaggtagcatcaaccaaa |
| | | | cagggaggt |
| 4091 | NM_003302 | TRIP6 | tctcagttccagttcccatcctttgattgatcactctccctgacatccacctgtatgactttgtca |
| | | | ccaa |
| 4092 | NM_003311 | PHLDA2 | ccttgattaatctttcaccgccccagcgcagacctgggaagcggaaaatcccgccgtatttt |
| | | | cccagccc |
| 4093 | NM_003315 | DNAJC7 | aaggcagttcagtttttcgtacaggctctcaggatggctcctgaccacgagaaggcctgca |
| | | | ttgcctgca |
| 4094 | NM_003318 | TTK | tgcatcttcaaatcactggcagattccggagttagcccgaaaagttaatacagagcagaaa |
| | | | cataccact |
| 4095 | NM_003352 | SUMO1 | actacgatactacaaacacaccacacaacccaactcaaaccacatcaatgcaatacatca |
| | | | acaaaatcac |
| 4096 | NM_003361 | UMOD | atgctgctttttctcaaaatgggacttgtgacggtgtacctgaggcccccatctccttaaaga |
| | | | gtgtggc |
| 4097 | NM_003383 | VLDLR | aaactaattttggtacgtatgaatgatatctttgacctcaatggaggtttgcaaagactgagtg |
| | | | ttcaaa |
| 4098 | NM_003390 | WEE1 | ctgtagtccggccctactcccacgtatccaacctagtctaccctagaatccactaacgcttta |
| | | | ttatgtt |
| 4099 | NM_003395 | WNT9A | agaccaaaaatgtctgctagctagttggtttctatacaaatacatattcgaatgtcttgctgga |
| | | | tgaaga |
| 4100 | NM_003403 | YY1 | gtctggtcagagttgctgagtgggttgatctctggtctttccttgacagtgcacgttcgaagg |
| | | | ctgtggg |
| 4101 | NM_003415 | ZNF268 | tgtatcggaagcatataccttggagggaccatgctatgagggaaagtgtaaatctagaaat |
| | | | gagaaaccc |
| 4102 | NM_003425 | ZNF45 | tgcagatctcgtggacaagtgtttatcaaactgaaggttgcagcttgttagtgggttcagaaa |
| | | | tcagttt |
| 4103 | NM_003427 | ZNF76 | gtatgccagcaaggttcttcatgacagccagattccccgtaatggaaaagggcagcaagtt |
| | | | ggagacaga |
| 4104 | NM_003453 | ZNF198 | accagatggttgtgtatgggtagcactactaaaagtttagaacttgcagtgtctttcggaattt |
| | | | ttaaaa |
| 4105 | NM_003467 | CXCR4 | actgtagaaaagggaactgaacattccagagcgtgtagtgaatcacgtaaagctagaaat |
| | | | gatccccagc |
| 4106 | NM_003480 | MFAP5 | ttaagaatatcttcacgaacttgaccctcctactccacattgcaacatttccatcagacagcat |
| | | | ttcaat |
| 4107 | NM_003549 | HYAL3 | ctacaggaggagagccactgtggtctgaagagatgctactgcctggagactggggtctta |
| | | | acctccagcc |
| 4108 | NM_003582 | DYRK3 | ggattccctctgaaaccctagatcggggtatatgttaagggattacgaaaatctaggactttt |
| | | | tgtgggg |
| 4109 | NM_003627 | SLC43A1 | ttctcagaccaagggacctggatgacaggcaatcaaggcctgagcaaccaaaaggagtg |
| | | | ccccatatggc |
| 4110 | NM_003636 | KCNAB2 | aagtccagtctgtgccggggaaggcactggttaggaaggatgttcaaacggtcccaccc |
| | | | aagcctgtcac |
| 4111 | NM_003645 | SLC27A2 | actgcttcggtatttatgcaactcaccacagaaaccaaatgaccgtgatcataaagtgaga |
| | | | ctggcactg |
| 4112 | NM_003692 | TMEFF1 | aagacagactttagtattctctatgtagtgccaagtaggcaaaagctcactcatgttcttattg |
| | | | cagcaa |
| 4113 | NM_003701 | TNFSF11 | ttggccaccaggtgcctttcaaatttagaaactaattgactttagaaagctgacattgccaaa |
| | | | aaggata |
| 4114 | NM_003707 | RUVBL1 | aagttatcgtaactgttcctgtggttgctttgaaagaacccttccttacctggtgtgttttctata |
| | | | aatc |
| 4115 | NM_003739 | AKR1C3 | aacagtcggtcttctatttcacacaagacttctcccgccgtattggttgcggaccttgcgcgg |
| | | | ccggagg |
| 4116 | NM_003746 | DNCL1 | cacaaagcgaggagtgacgcgataaaacgccgaacggaacacgaactgcgacactga |
| | | | aagaacccacccg |
| 4117 | NM_003753 | EIF3S7 | gagatgatattgaccttattgtccgttgtgagcacgatggcgtcatgactggagccaacgg |
| | | | ggaagtgtc |
| 4118 | NM_003801 | GPAA1 | aaagttacactgaggctccccccaccgatgctgcatacagatggtgtcgggcaccaacgt |
| | | | gtacggcatc |
| 4119 | NM_003805 | CRADD | gcctcttctaccttctaactccttgctttctaagccccaggtatctgataacttgattttgtcctg |
| | | | agtc |
| 4120 | NM_003821 | RIPK2 | ccatttttaacctcagccttccctactgtcaccaacaaccaagctaaataaagtcaacagcct |
| | | | gatgtgt |
| 4121 | NM_003824 | FADD | gtcgaaaacgcgctcttgtcgatttcctgtagtgaatcaggcaccggagtgcaggttcggg |
| | | | ggtggaatc |
| 4122 | NM_003842 | TNFRSF10B | ccagtcagtaggaaagtgccacaattgtcacatgaccggtactggaagaaactctcccatc |
| | | | caacatcac |
| 4123 | NM_003852 | TIF1 | ccaaacggtgcaacagaagggtaggttagatgctattaagaaggcacttaatagtacatca |
| | | | tgtaagatg |
| 4124 | NM_003874 | CD84 | ccatagactcatctagcaccaactaccattagcactatgttaggagctgcaaggccccaaa |
| | | | gtagaagat |
| 4125 | NM_003877 | SOCS2 | atgggaaacccacgggtacagaggactgaccatattaaagagttagggctactaacctca |
| | | | aaaagtttct |
| 4126 | NM_003879 | CFLAR | tcagggctccccctgcatcacatcaggaggatgttcatgggagattcatgcccttatctagc |
| | | | agggaagc |
| 4127 | NM_003884 | PCAF | acattagtatatgcgtccagggtcataaccccctaaaatccatcatgcaaccttattaatctgt |
| | | | cttggg |
| 4128 | NM_003900 | SQSTM1 | catcatcgaagtcttccccagttataaagaggtcacatagtcgtgtgggtcgaggattctgt |
| | | | gcctccag |
| 4129 | NM_003902 | FUBP1 | tcctccagcacctcagggccaataataagaagtggacaatacagtatttgcttcattgtgtg |
| | | | ggggaaaa |
| 4130 | NM_003914 | CCNA1 | gtaccttgatataccccatcgacctcagcaagcaattagggagaagtacaaggcttcaaag |
| | | | tacctgtgt |
| 4131 | NM_003954 | MAP3K14 | cttggtactgcagagtctgggttgtagagaactctttgtaagcaataaagtttggggtgatga |
| | | | caaatgt |
| 4132 | NM_003955 | SOCS3 | ttggggtgacctgaagggaaccatcctggtaccccacatcctctcctccgggacagtcac |
| | | | cgaaaacaca |
| 4133 | NM_003974 | DOK2 | tgtacgactggccctacaggtttctgcggcgctttgggcgggacaaggtaaccttttcctttg |
| | | | aggcagg |
| 4134 | NM_003998 | NFKB1 | tgtcattgctgttgtccctctgctacgttcctattgtcattaaaggtatcacggtcgccacctgg |
| | | | cattc |
| 4135 | NM_004039 | ANXA2 | caaagagaacgacgtcacgtccacaacccaaagcctcatgcgataccaggcgacatcat |
| | | | ctgtttatttc |
| 4136 | NM_004047 | ATP6V0B | gtgtgtccatcctggttagtcatatatctcttgtggtctgtccatccaaccatgtgctagatgtc |
| | | | atctt |
| 4137 | NM_004064 | CDKN1B | aagactgatccgtcggacagccagacggggttagcggagcaatgcgcaggaataagga |
| | | | agcgacctgcaa |
| 4138 | NM_004067 | CHN2 | actgcagaggatcgctgagtggggtactgtgtctcatagacatgcgccacctccacgtga |
| | | | gaacaagggt |
| 4139 | NM_004077 | CS | cccagtcccagaaccacactgttgagttggacacactgtaaacccctgggtaactgtcaag |
| | | | tcatgatgg |
| 4140 | NM_004083 | DDIT3 | gaccagggaagtagaggcgactcgccgagctctgattgaccgaatggtgaatctgcacc |
| | | | aagcatgaaca |
| 4141 | NM_004088 | DNTT | ggaagctcatcaaagcccactttgttcgcagtgtagctgaaatactgtctatctctaataaaa |
| | | | acaggag |
| 4142 | NM_004093 | EFNB2 | actcctcctcggaagagcccgtcgcgctggacagcttacctagtcttgtagcattcggcctt |
| | | | ggtgaaca |
| 4143 | NM_004099 | STOM | gcgggtgacatttgtaacatttcctctttgagactctgagttcacctagagaagtctaagcat |
| | | | aacagct |
| 4144 | NM_004104 | FASN | actttgtggtcttctcctctgtgagctgcgggcgtggcaatgcgggacagagcaactacgg |
| | | | ctttgccaa |
| 4145 | NM_004111 | FEN1 | gtaatttgtgtctagttattcagaggagtaagatggtgatgttcacctggcaatcagctgagtt |
| | | | gagact |
| 4146 | NM_004119 | FLT3 | catatctgagagcgttccagagccgatcgtggaatgggtgctttgcgattcacaggggga |
| | | | aagctgtaaa |
| 4147 | NM_004177 | STX3A | cccagtggtaaatcccttagatcccctgctggtctctggcagtctccttgattttgggtaccat |
| | | | gtatat |
| 4148 | NM_004208 | PDCD8 | caaagtggtcgtggggattgtgctatggaacatctttaaccgaatgccaatagcaaggaag |
| | | | atcattaag |
| 4149 | NM_004221 | NK4 | gatcccgcaaccgaggagcctaggggagagctttagtgacaaggtcatgagatggttcc |
| | | | aggccatgctg |
| 4150 | NM_004235 | KLF4 | gccaaagttttcaaactgctgcatactttgacaaggaaaatctatatttgtcttccgatcaaca |
| | | | tttatg |
| 4151 | NM_004310 | RHOH | aggaattgtcgaaaccgtggaaatctatttagctgaaatctagccagtgtgtagacaatgtt |
| | | | gaaatttt |
| 4152 | NM_004313 | ARRB2 | atcatcagagagatgatccgtctcctggatcagggtttaaacatcccagagaaaggggaa |
| | | | gttgctgact |
| 4153 | NM_004320 | ATP2A1 | gggcctcatgtcaactacagccagctgactcacttcatgcagtgcaccgaggacaacacc |
| | | | cactttgagg |
| 4154 | NM_004336 | BUB1 | agttctattctgcccacttattccagaatggcagtgtattagtaggagagctctacagctatg |
| | | | gaacatt |
| 4155 | NM_004343 | CALR | caccaacgatgaggcatacgctgaggagtttggcaacgagacgtggggcgtaacaaag |
| | | | gcagcagagaaa |
| 4156 | NM_004355 | CD74 | accatggagaccatagactggaaggtctttgagagctggatgcaccattggctcctgtttg |
| | | | aaatgagca |
| 4157 | NM_004374 | COX6C | gtgtggtatgcccaaaatggaaaagaggctattcagagtggtcagggagcttagaggagc |
| | | | gtaaaggaga |
| 4158 | NM_004380 | CREBBP | gaatgcacgtccaagaatacggctctgattgcccccctccaaacacgaggcgtgtgtaca |
| | | | tttcttatct |
| 4159 | NM_004385 | CSPG2 | tgagcatgacttccgttggactgatggcagcacactgcaatacgagaattggagacccaa |
| | | | ccagccagac |
| 4160 | NM_004413 | DPEP1 | atggtgaacttctacaacaattacatttcctgcaccaacaaggccaacctgtcccaagtggc |
| | | | cggtaggt |
| 4161 | NM_004425 | ECM1 | atacccttgacaaatactgtgaccgggagtatgctgtgaagacccaccaccacttgtgttgc |
| | | | cgccaccc |
| 4162 | NM_004430 | EGR3 | taaggtacttggttatccagtgcagtgcaccggaataaagagaatttgtaggcgtatacagc |
| | | | tttaaatg |
| 4163 | NM_004448 | ERBB2 | tggccatcaaagtgttgagggaaaacacatcccccaaagccaacaaagaaatcttagac |
| | | | gaagcatacgt |
| 4164 | NM_004449 | ERG | gaacgagcgcagagttatcgtgccagcagatcctacgctatggagtacagaccatgtgcg |
| | | | gcagtggctg |
| 4165 | NM_004454 | ETV5 | tgtgaagaggttcgcagggataaggggtgcactttatagctatggaaacatgagattctcct |
| | | | ctattgga |
| 4166 | NM_004475 | FLOT2 | tagagattatgacgttgcagccccgctgcgaggacgtagagacggccgagggggtagct |
| | | | ttaactgtgac |
| 4167 | NM_004494 | HDGF | tctctgaagccacaaataggatgcttgggaagactcctagctgtcctttttcctctccacaca |
| | | | gtgctca |
| 4168 | NM_004501 | HNRPU | tttgctgggtagtggagggggtaagctaaatcatagtttctgacaataactgggaaggtttttt |
| | | | cttaaa |
| 4169 | NM_004508 | IDI1 | tgctgtgcattgcaggatggtgagcagcgtccctctcatgtgacacccacagttatgccgg |
| | | | atgttgcca |
| 4170 | NM_004513 | IL16 | tggtgggggtgcagatgaggttgctagagaatgttagaggatccctctctggattggagat |
| | | | agggaaaga |
| 4171 | NM_004526 | MCM2 | cagttctgaggccctatgccatccataaggattccttgggattctggtttggggtggtcagtg |
| | | | ccctctg |
| 4172 | NM_004530 | MMP2 | cagcccacatagtgatggttcccctgttcactctacttagcatgtccctaccgagtctcttctc |
| | | | cactgg |
| 4173 | NM_004557 | NOTCH4 | aagaattgcaggcatatgggatgtaagatgttctttcctatatatggtttccaaagggtgccc |
| | | | ctatgat |
| 4174 | NM_004583 | RAB5C | taaggtggacgttttgaccgagtcgatgcgccgttcttgtgcctgtctacctgcgcgtttgct |
| | | | gtacgcg |
| 4175 | NM_004615 | TM4SF2 | tttaatgtagtactctgcttctgtatccccgaggtgagtcagaaaaatttcaagttgccacgcc |
| | | | ttggcc |
| 4176 | NM_004624 | VIPR1 | tacccacacctctgccagaagatcccctcaggactgcaacaggcttgtgcaacaataaat |
| | | | gttggcttgg |
| 4177 | NM_004633 | IL1R2 | atttcccccctcaagaccatatcagcttctctggggtcaagactgacaatcccgtgtaaggt |
| | | | gtttctgg |
| 4178 | NM_004642 | CDK2AP1 | ccgaacactttcatactaagactgtaagtagatctcagttctgcgtttattgtaagttgataaaa |
| | | | acatc |
| 4179 | NM_004688 | NMI | ttctctaaaagcacctacatcattgcttctacgcttgacgtggctctgagctcccaatctcctg |
| | | | ggacag |
| 4180 | NM_004689 | MTA1 | tgtcagtgagagatgttggaatatatggtccaatgaagcacatccctcgcgcccgacccgc |
| | | | cctggcagt |
| 4181 | NM_004706 | ARHGEF1 | acagactgtgtcggagcggaaaaactggtgtgctctcatcactgagactgccggatccct |
| | | | gaaagtccct |
| 4182 | NM_004725 | BUB3 | aacaaaaagcgactgtgccaattccatcggtaccccacgagcatcgcatcacttgccttca |
| | | | gtaatgatg |
| 4183 | NM_004815 | PARG1 | aagtccttaagttccaactctatttttgatccggattacatcaaggagttggtgaatgatatca |
| | | | ggaagt |
| 4184 | NM_004827 | ABCG2 | catctttaaaatgaataggtaggttagtagcccttcagtcttaatactttatgatgctatggtttg |
| | | | ccat |
| 4185 | NM_004867 | ITM2A | tttatttaccaactttgcaataacagaaagtccttccgccttcgtcgcagagacctcttgctgg |
| | | | gtttca |
| 4186 | NM_004924 | ACTN4 | tctcctgctctcataatgaagacatagccgattctctgcccgggccccttgctgatgctcctc |
| | | | cgggtct |
| 4187 | NM_004936 | CDKN2B | ggttcccttctctcaatgtttgtaataattgctcataagcaataccagcaattcataaaaactg |
| | | | cttact |
| 4188 | NM_004938 | DAPK1 | ttggtgcatagcagatggtttccacatttagatcctggtttcataacttcctgtacttgaagtct |
| | | | aaaag |
| 4189 | NM_004941 | DHX8 | ttcctgtggcaagtccacagtccgagtgcagaaggccatctgcagtgggttcttccgtaat |
| | | | gctgccaag |
| 4190 | NM_004972 | JAK2 | catcttgtgtgatgttttacacacatgagggctggtgttcattaatactgttttctaatttttccat |
| | | | agt |
| 4191 | NM_004994 | MMP9 | gagtgagttgaaccaggtggaccaagtgggctacgtgacctatgacatcctgcagtgccc |
| | | | tgaggactag |
| 4192 | NM_004997 | MYBPH | aatctgccaaggtgcccacagcagagcctcccggagaagtggcagtatcagagtccacc |
| | | | agagaagagca |
| 4193 | NM_005012 | ROR1 | aggagtagggccattgctagcccgtctctcactttggtgaaatttagaaaaaggcggttcct |
| | | | tttagaaa |
| 4194 | NM_005080 | XBP1 | ccagctgattagtgtctaaggaatgatccaatactgttgcccttttccttgactattacactgcc |
| | | | tggag |
| 4195 | NM_005084 | PLA2G7 | ctcaaattaaagggagacatagattcaaatgcagctattgatcttagcaacaaagcttcatta |
| | | | gcattct |
| 4196 | NM_005085 | NUP214 | aattgcccaaggaacgctcgagtctgcttgctgtgtccaacaaatatggtctggtcttcgct |
| | | | ggtggagc |
| 4197 | NM_005098 | MSC | agaggtctagacgtgagatccgcgtcgggacagggttttaagtgacaaagaagggcgag |
| | | | tggcttctctg |
| 4198 | NM_005104 | BRD2 | tacctgaggccatagctgccctattcacttctaagggccctgttttgagattgtttgttctaattt |
| | | | attt |
| 4199 | NM_005123 | NR1H4 | gggtctgcggttgaagctatgttccttcgttcagctgagattttcaataagaaacttccgtctg |
| | | | ggcatt |
| 4200 | NM_005157 | ABL1 | tgatagaaatggtttcctctggatcgttttatgcggttcttacagcacatcacctctttcccccc |
| | | | gacgg |
| 4201 | NM_005161 | AGTRL1 | gatgtctacactgccagagagaccgtgaggatgaaattaatagtggggcctttgtgagcta |
| | | | gaggctggg |
| 4202 | NM_005178 | BCL3 | aggaggggagaggtgggccgtaacgggcacggatcacgatgtaaattattaagcattttg |
| | | | gttggatttc |
| 4203 | NM_005187 | CBFA2T3 | gtgaatgtatatagtctttgcagaggtccaaatgatattcatgatggtaataaacgagatgttt |
| | | | gccaaa |
| 4204 | NM_005192 | CDKN3 | tttttatataccacgccacccatggggggtcttaacacgtgggggggcacccgataagca |
| | | | gctttcggta |
| 4205 | NM_005197 | CHES1 | cctgaagcccgatgacatcttacgaggtggacgttggactgttcatgcgcatcgggtgtca |
| | | | gtgactcat |
| 4206 | NM_005215 | DCC | acaatcagtggtgataggcttactcatcaaatcatggatctcaaccttgatactatgtattactt |
| | | | tcgaa |
| 4207 | NM_005225 | E2F1 | cgtggactcttcggagaactttcagatctcccttaagagcaaacaaggcccgatcgatgttt |
| | | | tcctgtgc |
| 4208 | NM_005230 | ELK3 | tctttaatgttgccaaacgctgccagtatttcatccgcctcacccttctcatctcggtccccgt |
| | | | ccctgt |
| 4209 | NM_005239 | ETS2 | atggggttcagcctaacagttatggaaactacagtccttataaaccattggcatggtaataa |
| | | | acagatct |
| 4210 | NM_005245 | FAT | catccactactcaattatagatggcaaccaaggaagctcgttcacaattgaccccgtcagg |
| | | | ggagaagtc |
| 4211 | NM_005246 | FER | ttcagaaagagctcactatcatcaagagaaaactcacatagtgacaggatggcgccaaac |
| | | | tcagccttca |
| 4212 | NM_005248 | FGR | gctcatcaccaagggccgaatcccctacccaggcatgaataaacgggaagtgttggaac |
| | | | aggtggagcag |
| 4213 | NM_005252 | FOS | tgtaatgttactgatcatgcattgttgaggtggtctgaatgttctgacattaacagttttccatga |
| | | | aaac |
| 4214 | NM_005263 | GFI1 | agagtgagaaccaagtgactccctaggcttcggacacaaataggctcctctacacctgaa |
| | | | gacaaaggca |
| 4215 | NM_005318 | H1F0 | cttttcttttcctctggatttttgttcctcctgtacaagaggtgtctttgcttggtttggtggggctg |
| | | | cg |
| 4216 | NM_005334 | HCFC1 | caccgttcactattgtagagtaacccctgtgactcaatattaccatagtgcgatgtcgttttgt |
| | | | gctatt |
| 4217 | NM_005335 | HCLS1 | tgctctgtagtatcctgtgtccataccagcctgtttctacattaaacacctatactcagcctga |
| | | | agcccc |
| 4218 | NM_005339 | HIP2 | tttcttcagtgtctacatattatgtcacacgttctatttgcactgctgcaatatagatcaaagatc |
| | | | ttgt |
| 4219 | NM_005340 | HINT1 | tgtgctgctgatctgggcctgaataagggttatcgaatggtggtgaatgaaggttcagatgg |
| | | | tggacagt |
| 4220 | NM_005356 | LCK | cccacatgtgacacatatgcaccttgtgtctgtacacgtgtcctgtagttgcgtggactctgc |
| | | | acatgtc |
| 4221 | NM_005375 | MYB | tgatgcaagatggccagcactgaacttttgagatatgacggtgtacttactgccttgtagca |
| | | | aaataaag |
| 4222 | NM_005385 | NKTR | aggagtactgtctcatctcatcatattcacgggttctggagattagctcattgaaagtgggag |
| | | | gggcatt |
| 4223 | NM_005424 | TIE1 | aagccaggaaggcctatgtgaacatgtcgctgtttgagaacttcacttacgcgggcattgat |
| | | | gccacagc |
| 4224 | NM_005427 | TP73 | ggattggggtgtccaaactgcatcgagtatttcacctcccaagggttacagagcatttacca |
| | | | cctgcaga |
| 4225 | NM_005439 | MLF2 | accctttgctattcaccgtcagcatatgagccgaatgctgtccggtggctttggatatagccc |
| | | | cttcctc |
| 4226 | NM_005494 | DNAJB6 | ccccacgggagcattgtagcacgcgctgtatggttgtgcgttgtagcgtgcgctgtatagtt |
| | | | gtgatgcc |
| 4227 | NM_005504 | BCAT1 | atgtttcctggggtgacttaacaaggaataatcccacaatatacctagctacctaatacatgg |
| | | | agctggg |
| 4228 | NM_005521 | TLX1 | cctgttgagtgacagtgtttcgcagctgagcccttaaggagattctcagttgggcagagac |
| | | | atcccttcc |
| 4229 | NM_005529 | HSPG2 | cctcggatgctggggtctacatttgcacctgccgtaatctccaccaatccaataccagccg |
| | | | ggcagagct |
| 4230 | NM_005533 | IFI35 | tcctatacctctatctgtcgtgcttaaacctctgctgctcgcgtactcgtcaccgtgcgcatca |
| | | | gtgagc |
| 4231 | NM_005556 | KRT7 | cccatctcccggccaaagctggtgccactgtctcagacccccttgtgagatctccagcaca |
| | | | gaatgttct |
| 4232 | NM_005563 | STMN1 | gtattttatgatagggataattcaggtcctaattttggtgttatttaaagggaccttattttctgcc |
| | | | cct |
| 4233 | NM_005572 | LMNA | gatcaagcgccagaatggagatgatcccttgctgacttaccggttcccaccaaagttcacc |
| | | | ctgaaggct |
| 4234 | NM_005583 | LYL1 | agtgacaaatcattccacacgataccccttgcattccagtagcgaaggttgttaccgccctc |
| | | | ctaccctt |
| 4235 | NM_005589 | ALDH6A1 | tagggaactgcctagtgtaacaatgaaaccagatttctcacttgctcttcatacttctattttga |
| | | | ggtaa |
| 4236 | NM_005608 | PTPRCAP | tccatttttaatcccgccaacgcacaatctccggaggaatgcccacgagctctcgcgttggt |
| | | | ggcgttta |
| 4237 | NM_005621 | S100A12 | gagcatctggagggaattgtcaatatcttccaccaatactcagttcggaaggggcattttga |
| | | | caccctct |
| 4238 | NM_005651 | TDO2 | ccacgatgctctgatttaattctagaaacaatttgattacctcttgtttgtgacaagactaagca |
| | | | ttaag |
| 4239 | NM_005654 | NR2F1 | aggagggccgagacaggagcagcccacccagcagaaatacaatccgagctacaaagc |
| | | | atgggaaaaagag |
| 4240 | NM_005671 | D8S2298E | taaaaacttggaagaagcttcaaagctcttggaggctttaaagttctttctgttgggtgtgcat |
| | | | tacagt |
| 4241 | NM_005687 | FARSLB | tgtgtgggtagagaccatcctttccctgcatattaggccagcttgtgccatataccagtgtgg |
| | | | tgtctgt |
| 4242 | NM_005730 | CTDSP2 | attcccacttgccccagagaagctactcggccaaacctagccagggtctgttcttgtggac |
| | | | cagagccag |
| 4243 | NM_005731 | ARPC2 | attaaagagggtgtacgggagtttcttggtaaatccagaatcaggatacaatgtctctttgct |
| | | | atatgac |
| 4244 | NM_005738 | ARL4A | attgaccagctaggctatttagtcccatagtagttcagatctgcttagatgtgacaggcattg |
| | | | aaggctg |
| 4245 | NM_005761 | PLXNC1 | tgtagtaacatgcagccgaaactcatgctgagacgcacggagtccgtcgtcgaaaaactc |
| | | | ctcacaaact |
| 4246 | NM_005767 | P2RY5 | actgtgatcggaggaagtgcacccgccgtttttgttcagtctacccactctcagggtaacaa |
| | | | tgcctcag |
| 4247 | NM_005781 | TNK2 | actttgggctgatgcgagcactacctcagaatgacgaccattacgtcatgcaggaacatcg |
| | | | caaggtgcc |
| 4248 | NM_005794 | DHRS2 | ttgtgagtatgccttaaattctatggggacaaggtaagaggacaagcaaatgaaagctcag |
| | | | agagacaaa |
| 4249 | NM_005808 | CTDSPL | gaattgaccagcggctgttacactgttctttgccactgtgcctatgctcagaatatgctcact |
| | | | gctaagc |
| 4250 | NM_005809 | PRDX2 | taatagaaaaagtcgaacacgctacagctaacttcttccgttatgagagcttgagactgcgc |
| | | | cgccgctc |
| 4251 | NM_005826 | HNRPR | aggccggacccatttgggatctacgtcttatgatgaatccactgtccggtcagaatagagg |
| | | | gtatgcatt |
| 4252 | NM_005835 | SLC17A2 | gtagccatatgttacagatctcagcaccgatcagaactgtaaagctataatccccagaatta |
| | | | aagttttt |
| 4253 | NM_005845 | ABCC4 | ccacactaaggaatcgtaccttctcagagtcttcggtttggtctcaacaatcttctagaccct |
| | | | ccttgaa |
| 4254 | NM_005870 | SAP18 | ttatgtagtagtatgatacctgtacttaagcatcactcaagggtaagtgttctaggaaggttga |
| | | | tttagc |
| 4255 | NM_005895 | GOLGA3 | ggcgtgttcatccccacaccgtgtgtcacaacaggctagggcacttcacgatgtcactactt |
| | | | gtttttct |
| 4256 | NM_005900 | SMAD1 | tcctgctattctgaaattgcctacatgtttcaataccagttatatggagtgcttgaatttaataag |
| | | | cagt |
| 4257 | NM_005902 | SMAD3 | gggctttgaggctgtctaccagttgacccgaatgtgcaccatccgcatgagcttcgtcaaa |
| | | | ggctgggga |
| 4258 | NM_005907 | MAN1A1 | gctgttcaggctatcgagactcatttgatccgcaagtctagcagcggactaacttatatcgc |
| | | | agagtgga |
| 4259 | NM_005911 | MAT2A | ctggtgcttatgctcttaattgccacctctaacagcaccaaatcaaaatctctccactttcagc |
| | | | tgtctt |
| 4260 | NM_005923 | MAP3K5 | attatacactattggatgttctctactatgttacacgtgatgacttaaaatgcttgagactaagg |
| | | | ggagg |
| 4261 | NM_005933 | MLL | cacccttttctcgccgcaagaagcccattcctatggaagtctagcaaagcaatacgactca |
| | | | gcccagcac |
| 4262 | NM_005936 | MLLT4 | gagaagataaagcttaccaacttgagcggcatcgaatagaggcagctatggaccgaaag |
| | | | tctgatagtga |
| 4263 | NM_005944 | CD200 | ggaggcaaatatgagcatacaatccctttgttctaaagatattgttccagctagtggaatgat |
| | | | gttgaat |
| 4264 | NM_005954 | MT3 | gcagaattccgtttttcaccggggcgtggctaccctgtcgtgacgcgtttccgcgtattgtcc |
| | | | cctataa |
| 4265 | NM_005956 | MTHFD1 | ttctgcccattcgcgacatccgcgccagcgttggggctggttttctgtaccccttagtagga |
| | | | acgatgag |
| 4266 | NM_005957 | MTHFR | gtgaaaacatcaccaatgcccctgaactgcagccgaatgctgtcacttggggcatcttccc |
| | | | tgggcgaga |
| 4267 | NM_005965 | MYLK | gcgttattgatgggcattaatctatgttagttggcaccttaagatactagtgcagctagatttc |
| | | | atttag |
| 4268 | NM_006005 | WFS1 | cctttctgagtgacatgggtgtgccaggctagactaggaggttccggtgtctggaaaagca |
| | | | ctttacaga |
| 4269 | NM_006017 | PROM1 | tacagcatattcttcacgcagagattttcatctattatactttatcaaagattggccatgttccac |
| | | | ttgg |
| 4270 | NM_006038 | SPATA2 | tctaaccaaccgtgatgccacgtaacttaccctatggacaaaagcaggaatgactaacac |
| | | | atcctaggag |
| 4271 | NM_006060 | ZNFN1A1 | ggtgtgtggagattctaatcccaacaagcaagggtctccttcaagattaatgctatcaatcat |
| | | | taaggtc |
| 4272 | NM_006079 | CITED2 | gcagatattactgcacaaactgccatctcgcttcatttttttaactatgcatttgagtacagact |
| | | | aattt |
| 4273 | NM_006082 | K-ALPHA-1 | tccacttgacagtcatcaatagattggtttaaattgtgatcttttttccttcccgcagcgtgagt |
| | | | gcatc |
| 4274 | NM_006084 | ISGF3G | ctttgtgataattctcagtagttgtccgtgataatcgtgtcctgaaaatcctcgcacacactgg |
| | | | ctggtg |
| 4275 | NM_006098 | GNB2L1 | ccaccacttacaagctggctgttgaaatgagagcggtttcttacagtctacccggcgttgtg |
| | | | gcacatgc |
| 4276 | NM_006101 | KNTC2 | ggagcagattgctaaagttgatagagaatatgaagaatgcatgtcagaagatctctcggaa |
| | | | aatattaaa |
| 4277 | NM_006113 | VAV3 | tgttatggagggagcagcctcacacaagcagaaacactcctgtggatggtattgtagcatg |
| | | | tattgttta |
| 4278 | NM_006120 | HLA-DMA | cacctgagctataccaacagtctcctaaattatttgctagacacatggctatttggatttaaatt |
| | | | aatac |
| 4279 | NM_006152 | LRMP | gccgctcccacacagcaagaggactcatggacgtctctagaacatatcttgtggccattta |
| | | | ccagactcc |
| 4280 | NM_006184 | NUCB1 | tcaagaccttcccctaccttttgtggaaccagtgatgcctcaaagacagtgtcccctccaca |
| | | | gctgggtg |
| 4281 | NM_006185 | NUMA1 | aaacaaagagctgcgagctgaagctgaacggctgggccatgagctacagcaggctggg |
| | | | ctgaagaccaag |
| 4282 | NM_006187 | OAS3 | tgggacatgcatgctatggggaccctcttgttggacacctaattggatgcctcttcatgaga |
| | | | ggcctcct |
| 4283 | NM_006190 | ORC2L | ggaacaatacgtcatatatttggaacagttattgtctgtgccatgaccttcatgataccagtga |
| | | | gaagcc |
| 4284 | NM_006191 | PA2G4 | aatcaggagtcaaaattcatcgtcttcaagcccctctttctagccttttctactactctctgcttg |
| | | | gtca |
| 4285 | NM_006196 | PCBP1 | ttgggcaagtttggatgcatctactcaaaccacccatgaactcaccattccaaataacttaat |
| | | | tggctgc |
| 4286 | NM_006209 | ENPP2 | cagaaatcctgacactcaagacatacctgcatacatatgagagcgagatttaactttctgag |
| | | | catctgca |
| 4287 | NM_006216 | SERPINE2 | tttaaaaaactcctgtcttgctagacaaggttgctgttgtgcagtgtgcctgtcactactggtc |
| | | | tgtact |
| 4288 | NM_006230 | POLD2 | tggcacattttcagcgttggctgagttacatgtacagtgcttaggccacctgggggagagta |
| | | | agagtggg |
| 4289 | NM_006231 | POLE | actgtcaggcgccctacgactcctctgccatcgagatgacgctggtggaagttctacagaa |
| | | | gaagctgat |
| 4290 | NM_006235 | POU2AF1 | aggtttgtaggagactgagttgttagcaggtgtgcttagctcttgatagtgaacgtgtacctt |
| | | | gggaact |
| 4291 | NM_006255 | PRKCH | aaactcacaacctaaagcccaatcttgaaaatatgttgtgcaccaagacgactgcttcagct |
| | | | tcttctct |
| 4292 | NM_006263 | PSME1 | acacaacccgcgttgtgaataattggttgtttgggccggtgaatacaccggccacgcaaca |
| | | | agtttacca |
| 4293 | NM_006268 | DPF2 | gaaaggagcaacacactgcccctaggcgtgcgtgtggcccagtttctctctgctctccatt |
| | | | aagtgcatt |
| 4294 | NM_006286 | TFDP2 | catgtaatattcttagaattgagattgctacacggagcagtccttgacgatcgtgtttctgacg |
| | | | tttctc |
| 4295 | NM_006299 | ZNF193 | ctgtagcaaggttctactgttttcagtttcatattacccctatatgcatcaaagctgcaacttcc |
| | | | cttgc |
| 4296 | NM_006301 | MAP3K12 | ctgcccaagccctgtcttaaggcccagggggaggttaggagactgatagcatgtgatggc |
| | | | tcaggctgaa |
| 4297 | NM_006302 | GCS1 | cttggtggaattggctacttctacggacaagggctggtattgccagacatcggggtggaag |
| | | | ggtctgagc |
| 4298 | NM_006325 | RAN | agccacaaacccaagcgaggaccaggacacaccacagccgatgtcgacacaaaactc |
| | | | agacgcaaagcaa |
| 4299 | NM_006329 | FBLN5 | tagtttgctttttgcgtacacagagaaggctatgtaaacaaaccacagcaggatcgaaggg |
| | | | tttttagag |
| 4300 | NM_006343 | MERTK | aaactcttagaaagtttgcctgacgttcggaaccaagcagacgttatttacgtcaatacaca |
| | | | gttgctgg |
| 4301 | NM_006355 | TRIM38 | agcggcagtcctggaggctaccagacttactgagttctacctgagaaacagccaagcaaa |
| | | | gtgtgagaga |
| 4302 | NM_006362 | NXF1 | ggtccccgagtacgatacaacccctataccacccgacctaaccgtcggggtgatacttgg |
| | | | catgatcgag |
| 4303 | NM_006427 | SIVA | ggtcgtgttgccttagggcccccatgtcagtgccgtcatctagactgattatgcccgcagtg |
| | | | atcctgtc |
| 4304 | NM_006472 | TXNIP | gcactttgttcactgtcctgtgtcagagcactgagctccacccttttctgagagttattacagc |
| | | | cagaaa |
| 4305 | NM_006495 | EVI2B | ctgagataatacaatcatttccaccgcttgactcacttaacttgcccctgccaccagtagattt |
| | | | tatgaa |
| 4306 | NM_006509 | RELB | tgaacagctctgacccccatggcatcgagagcaaacggcggaagaaaaagccggccat |
| | | | cctggaccactt |
| 4307 | NM_006534 | NCOA3 | caagggattagaccacctatggctggatgcaacagttcggtaggcggcatgagtatgtcg |
| | | | ccaaaccaag |
| 4308 | NM_006559 | KHDRBS1 | ttttgtccatttgagattctgcactccatgaaaagttcacttggacgctggggccaaaagctg |
| | | | ttgattt |
| 4309 | NM_006565 | CTCF | gtgtattgcttttaagagagccatcagttagctatcagactctaggttgatgcattttgtactta |
| | | | gctgt |
| 4310 | NM_006579 | EBP | gacgtggacaacttgtgtacctaagtgaccgccccacctgtggcttatacgtggcgtggcc |
| | | | tctttcttc |
| 4311 | NM_006591 | POLD3 | accttcaagtgacttccattatggctggacaggcggtgagctcagtggattgcagtggtgtg |
| | | | ctggtgat |
| 4312 | NM_006602 | TCFL5 | tctgcggaccgggtgaccgtgcttctgtggaatgggtgaatatacttctgcggaccaggtg |
| | | | actatgctt |
| 4313 | NM_006676 | USP20 | aagactctggactcattgctgattggaacaccaggaggaggttggatttctgccagtgggg |
| | | | gatgtttct |
| 4314 | NM_006739 | MCM5 | caccatcttgtcgcgcttcgacatgatcttcatcgtcaaggatgagcacaatgaggagagg |
| | | | gatgtgatg |
| 4315 | NM_006747 | SIPA1 | cgatggtttgcccactatgacgtgcaaagcctgctctttgattgggctccgaggtctcaggg |
| | | | gatgggga |
| 4316 | NM_006748 | SLA | gcagatgggtgccctgaatggaactactttaactaatccatagggacttctggtatgctttcc |
| | | | tctcttt |
| 4317 | NM_006759 | UGP2 | gagaatgaaaaacgaggaattggagatatgaatatcgaaaactcaagagttttgccttaaa |
| | | | gagaaaca |
| 4318 | NM_006762 | LAPTM5 | cacaactgggtcaacgctttaggctgagtcactcctcgggtctctccataattcagcccaac |
| | | | aatgcttg |
| 4319 | NM_006769 | LMO4 | tgcagcgcttgcggacagtcgattcctgcgagtgaactcgtcatgagggcgcaaggcaat |
| | | | gtgtatcatc |
| 4320 | NM_006803 | AP3M2 | aagtgtttaccgccacaagatcttttttgtggccgtgatccagacggaggtcccccctctgtt |
| | | | tgtcatt |
| 4321 | NM_006820 | IFI44L | tttgagtcttctctataagtctagtgttcatggaggtagcattgaagatatggttgaaagatgc |
| | | | agccgt |
| 4322 | NM_006825 | CKAP4 | aaggggagtccttgggttctttcaaaaaattcgtgaggagagctgtctacagtggaatagg |
| | | | gggtctccc |
| 4323 | NM_006845 | KIF2C | cctgtttcccggtctcgctatcaagatccaacgcagtaatggtttaattcacagtgccaatgt |
| | | | aaggact |
| 4324 | NM_006857 | RY1 | gcagtacatgaatcgaaaaggtggattcaacagacctttggatttcattgcatgagaattga |
| | | | agtgttga |
| 4325 | NM_006875 | PIM2 | tcccaagtgctcttattctggtgagaagaaccttacttccataatttgggaaggaatggaaga |
| | | | tggacac |
| 4326 | NM_006904 | PRKDC | tggagactggcggcgtgatcgggatcgactttgggcatgcgtttggatccgctacacagtt |
| | | | tctgccagt |
| 4327 | NM_006910 | RBBP6 | tggatatcatcgatctaggtcaaggtcacccccttacagacgctatcattcacgatcaagat |
| | | | ctcctcaa |
| 4328 | NM_006931 | SLC2A3 | tggaatcacccctagatctttcttgaagacttgaattagattacagcgatggggacacagaa |
| | | | ggtcaccc |
| 4329 | NM_006932 | SMTN | agagcacggactttgaagagcggaagctcatccgggctgcacttcgtgagctccgacaa |
| | | | aggaagagaga |
| 4330 | NM_006937 | SUMO2 | ccatgttaatatgcaagtatactagcagaaaaattgggccatagtatcgtggatttccaggta |
| | | | ttttctg |
| 4331 | NM_006938 | SNRPD1 | tgtacagctggaaacgctgagtattcgaggaaataacattcggtattttattctaccagacag |
| | | | tttacct |
| 4332 | NM_006963 | ZNF22 | ggccggcagcggcagaagtggggcatgactattcgatttgactcaagcttcagtagactc |
| | | | agaagaagct |
| 4333 | NM_006981 | NR4A3 | tccgtgttcgtactcccttttgtatgtttctactgttaatgccatattactatgagataatttgttgc |
| | | | at |
| 4334 | NM_007022 | CYB561D2 | tggtcatcatgaaccaggtgagcaatgcctacctataccgcaagaggatccaaccatgag |
| | | | ctcttcccag |
| 4335 | NM_007063 | TBC1D8 | gccctaaaccgagatttcagtctaccttaccaatggggcatctggctaaacaacctgacaa |
| | | | actgtggcc |
| 4336 | NM_007065 | CDC37 | aaatacgagaaacagatcaagcactttggcatgcttcgccgctgggatgacagccaaaag |
| | | | tacctgtcag |
| 4337 | NM_007079 | PTP4A3 | cctgttctcggcaccttaaattattagaccccggggcagtcaggtgctccggacacccgaa |
| | | | ggcaataaa |
| 4338 | NM_007117 | TRH | gagatgtgtacgcttatgtcggtgtcccaacgtataaactatcacagtagcgctcatggctc |
| | | | gtaggttt |
| 4339 | NM_012092 | ICOS | ataggttgctgtcagactatacccatggtgcctctgggcttgacaggtcaaaatggtcccca |
| | | | tcagcctg |
| 4340 | NM_012120 | CD2AP | tcatcctaataatgggatgagggaagtttccagcagatttcaggctgttcttaaagtttttgttg |
| | | | gtcat |
| 4341 | NM_012207 | HNRPH3 | ggaggttctggaatgggaggctacggaagagatggaatggataatcagggaggctatgg |
| | | | atcagttggaa |
| 4342 | NM_012223 | MYO1B | ttaggtgtatcaaaccgaatgataaaaaagcagcacacatcttcaacgaggctctagtgtgt |
| | | | catcagat |
| 4343 | NM_012267 | HSPBP1 | ttccataaggggggcttacacagttgccctaatgcggctggaatccccattgggggtaggt |
| | | | gagcaggca |
| 4344 | NM_012323 | MAFF | gaaactgctcttattgtgccaatatgccctccaaaccctcccaggattcaaagctaggtttgg |
| | | | ctgtctg |
| 4345 | NM_012334 | MYO10 | acacgaaaaaagaaatttgccttgtccctttgtgtacaaccatgcaaaactgtttgttggctc |
| | | | acagaag |
| 4346 | NM_012384 | GMEB2 | gaggcataaacagaaccagtcactatggaggtcgaggattcccatgtgggcccttcagct |
| | | | tgggctgctt |
| 4347 | NM_012417 | PITPNC1 | tgagatatccaggagattcattaggtcatcagcgatcaaaggttagcattttattatagatgtt |
| | | | cactgt |
| 4348 | NM_013282 | UHRF1 | tgttctacaggggcaaacagatggaggacggccataccctcttcgactacgaggtccgcc |
| | | | tgaatgacac |
| 4349 | NM_013314 | BLNK | ttgttctattgcctgacctgatgaactgttaatatctggtgaggttgagttatcatgctactaata |
| | | | tttt |
| 4350 | NM_013416 | NCF4 | gcggtagatgaggtacttggatcctccttttgtcttcacctcgatgacgaaaacctaaggag |
| | | | gacagagg |
| 4351 | NM_014005 | PCDHA6 | tacagatgtcatttgtctcccgccctaagtcctccagtctccttagagctagtacttactaagc |
| | | | atttac |
| 4352 | NM_014207 | CD5 | cttgacgaaaacagactcctctagtacttggagatcttggacgtacacctaatcccatgggg |
| | | | cctcggct |
| 4353 | NM_014232 | VAMP2 | tgaggaagggtctattgtctttttaaatggcacaattttaagggtttgagggtacagtccctta |
| | | | acctgc |
| 4354 | NM_014244 | ADAMTS2 | tgaatttgtgaacaatacaccctctccgcccttggggtaatgtactttgtgatattgccacccg |
| | | | cccccg |
| 4355 | NM_014298 | QPRT | tgggtctaataaaggatcaaccacatggggttctgcggtgataatgagcacatagtgaggg |
| | | | gtcagcaaa |
| 4356 | NM_014345 | ZNF318 | acttttagaagacgacaaatcggtaaggcttattcatggttcattgccaggagcagatatgct |
| | | | tttgaga |
| 4357 | NM_014365 | HSPB8 | gcctaaaatgggtgatatacaggtcttatatccccatatggaatttatccatcaaccacataa |
| | | | aaacaaa |
| 4358 | NM_014575 | SCHIP1 | caaaacagcaccagcaattatgtgacctgctttcgatcataagactgtgttaagaccttctcc |
| | | | tcagtgt |
| 4359 | NM_014595 | NT5C | gtgtggtttcccccggaaagatgattttgatcaagcacaggtaatcaggactgtcctcagga |
| | | | ctttatgt |
| 4360 | NM_014614 | PSME4 | ttgcagattcacatgaaagcacccagcgatgtgttgcagaaattatagctggtttaatcaga |
| | | | ggttctaa |
| 4361 | NM_014624 | S100A6 | gcagtctgatcgatcgcgatgtaagtcgcgagccggcaatcgcgtgtgcacatactcgca |
| | | | gatccttctc |
| 4362 | NM_014667 | VGLL4 | gctgcctgcagtgcgctctgaccttctcttcatgtgtgtaaatctgtaatataccattctctgtg |
| | | | gcctg |
| 4363 | NM_014707 | HDAC9 | ctggtcacgactgcaagaaactgggctgctaaataaatgtgagcgaattcaaggtcgaaa |
| | | | agccagcctg |
| 4364 | NM_014724 | ZNF305 | tttctgtaaatttgagactacctttatattctccaacacaagaccgtgtaggtgtcccttcagag |
| | | | agtat |
| 4365 | NM_014734 | KIAA0247 | atgtcgcctccatttccgaggaacatccttgcgtagagaatgaaatatgctgcaatcatttct |
| | | | gcatcct |
| 4366 | NM_014735 | PHF16 | atgtttcaagtctagtttttacaagcacattacagtaattgcaggttgtccagaggttggtttgt |
| | | | cagag |
| 4367 | NM_014780 | CUL7 | agatggcagcaaggaacccaggtcgagatgggacattgtgcggctcatcccacctcaga |
| | | | cgtacctgcaa |
| 4368 | NM_014792 | KIAA0125 | attcacatgaacgggcacatacaggaaaacacggtaatgtaattagaatagtcagagaaa |
| | | | agtagccaga |
| 4369 | NM_014824 | FCHSD2 | ctgtccgctcctgtagaaggtagggagcacaatacctagggactaagggatgttcgccgg |
| | | | ttgtggtttg |
| 4370 | NM_014890 | DOC1 | cagtttcagcgttcaaacagcaatagctcaagtgtgataactactgaggataataaaatcca |
| | | | cattcact |
| 4371 | NM_014899 | RHOBTB3 | cctagacatggaaaaacagattactgtctattgtcagcatcattttcatctgtaagtcactact |
| | | | ggaata |
| 4372 | NM_014914 | CENTG2 | ctaatacatggagttgtgcttcctaagtattgatagcggcgtatatataggattcccacaattt |
| | | | gcagtt |
| 4373 | NM_015050 | KIAA0082 | tttttcttccctaataggaggtacaatctgcttttgtttgtcgttaagtggtcactcccatttccttt |
| | | | at |
| 4374 | NM_015069 | ZNF423 | catttgcaacccagatgtaaaactctaatgatttggccatgaggcgctgctattataagcag |
| | | | ctggaaat |
| 4375 | NM_015099 | CAMTA2 | ggctccacgggcactttctacttgtgcatgggcttggtttatacgaattgccattaaacatcg |
| | | | ctgcacc |
| 4376 | NM_015147 | KIAA0582 | taaggcatgctgaaatttgttgtaaatcccaatctttactgccatggggaaaagaacgctgg |
| | | | aattttcc |
| 4377 | NM_015149 | RGL1 | tgtttcctggtggacagccgggtaatgcttttagctgctcgcatgcttgtctttctgcatctcca |
| | | | tcatc |
| 4378 | NM_015158 | ANKRD15 | atgcccatgtcaactttgcaaaagcccagtctccgggcacccctaggcttggaaggaaga |
| | | | cgtctcctgg |
| 4379 | NM_015166 | MLC1 | ctgatctgagaagacatgaacgttttctcttcaccgccgtggggtgtattgactggtccccc |
| | | | atgggctg |
| 4380 | NM_015196 | KIAA0922 | tctggactttttggttccatctgggccccgcaaagcgatgtgtatgaaaattgctgccccatc |
| | | | aacccca |
| 4381 | NM_015210 | KIAA0802 | ctccggaggtccatctccgagatcgaagaccacaaccggcaactgacccacgagctcag |
| | | | caagtttaagt |
| 4382 | NM_015261 | KIAA0056 | agtggtctctagaggtgatcagttcagctgttgacgccttgcagaggctttgtagagcatct |
| | | | gcagagac |
| 4383 | NM_015335 | THRAP2 | ccagattgcaaagagttatggatattctggtggggagatgaacccaacctagtgggtgtaa |
| | | | tacatcatg |
| 4384 | NM_015436 | RCHY1 | aaatatgtttgatgatccagccgactttgacactgttatctccgtaatagctttaccattaggct |
| | | | aggat |
| 4385 | NM_015458 | MTMR9 | aacttgtgagggagtttgtattaggtctggtcatggagggatgatgtaccatcaaggtagac |
| | | | ccatcttc |
| 4386 | NM_015474 | SAMHD1 | gatttattccacttgctcgcccggaggtgaaatggacgcatgaacaaggctcagttatgatg |
| | | | tttgagca |
| 4387 | NM_015528 | RNF167 | tgatgaaggggagccaagggaccaccctgcctcagaaaggaccccacttttgggttctag |
| | | | ccccactctt |
| 4388 | NM_015568 | PPP1R16B | ctcagccagaacctcttggtgtacccgataagctgcaggttatcccttgctctgtgcgccttt |
| | | | tatttgt |
| 4389 | NM_015570 | AUTS2 | caagtctgggaacagccgtaacaggtcaaccttgtggagccatcgcgagttagagggtg |
| | | | aaagatggcag |
| 4390 | NM_015636 | EIF2B4 | gcggggagaacatgttgcgctggctaactggcagaaccacgcatccctacggttgttgaa |
| | | | tctagtctat |
| 4391 | NM_015670 | SENP3 | cgctgccctaagcatattgccaagtatctacaggcagaggcggtaaagaaagaccgact |
| | | | ggatttccacc |
| 4392 | NM_015710 | GLTSCR2 | cgtgagatccagttgtagctgccatcagatgccggagactcgcccttcaataaaaaatctct |
| | | | tctagctg |
| 4393 | NM_015833 | ADARB1 | atgaactgaacggttaaaagcacagtctatggaacgctaatggagtcagcccctaaagct |
| | | | gtttgctttt |
| 4394 | NM_015855 | WIT-1 | gctccccgagagtcctcagtgaaaggattccgcggcactgcctctattattataccgtaaat |
| | | | ctttttaa |
| 4395 | NM_015869 | PPARG | aataaggaggttctttatggaataaacaggtattaagctatttaaaggagagggctgcgaaa |
| | | | tgcttgat |
| 4396 | NM_015965 | GRIM19 | aaatcgaggacttcgaggctcgcatcgcgctgttgccactgttacaggcagaaaccgacc |
| | | | ggaggacctt |
| 4397 | NM_016041 | DERL2 | taacatacctccaacaccgtcatacatatacctccgctacgcaaccttacctagttacgccc |
| | | | caattgta |
| 4398 | NM_016091 | EIF3S6IP | ttcgctctttccggcggtgctcgcaagcgaggcagccatgtcttatcccgctgatgattatg |
| | | | agtctgag |
| 4399 | NM_016184 | CLECSF6 | ttcttattcatgtgtaagggaggtccatagaatttaggtggtctgtcaactattctacttatgag |
| | | | agaat |
| 4400 | NM_016195 | MPHOSPH1 | tggcagtgaaacaccctggttgtaccacaccagtgacagttaagattcccaaggctcgga |
| | | | agaggaagag |
| 4401 | NM_016221 | DCTN4 | atgataacccaaaggtagacctgctgttaatgatccagcattggtcacaatgtaccaactgc |
| | | | tttctgca |
| 4402 | NM_016308 | UMP-CMPK | caagagtaaagactaaggtttcgagagcattcctactcacataagtgaagaaatctgtcag |
| | | | ataggaatc |
| 4403 | NM_016343 | CENPF | gctggagatagaccttttaaagtctagtaaagaagagctcaataattcattgaaagctactac |
| | | | tcagatt |
| 4404 | NM_016570 | PTX1 | aagttacttaaacctgagctttaggacctttaaaatctcagcatgccttttaagctagtactttt |
| | | | cctgt |
| 4405 | NM_016734 | PAX5 | aggcccaataatatttggaccaagtgggaaacaagaacactcggaggggcgggaatca |
| | | | gaagagcctgga |
| 4406 | NM_017437 | CPSF2 | ctttctgtatttgcgtctttcttgtgactaaccaccctgatatagtattaaccactgtgttcaaga |
| | | | gtaa |
| 4407 | NM_017448 | LDHC | tgtgtcttggggcggaatggtgtctcagatgttgtgaaaattaacttgaattctgaggaggag |
| | | | gcccttt |
| 4408 | NM_017617 | NOTCH1 | tgtcaaacatgagatgtgtggactgtggcacttgcctgggtcacacacggaggcatcctac |
| | | | ccttttctg |
| 4409 | NM_017771 | PXK | catgagccgaggggccttgctcagctccatccagaatttccaaaaaggaactttgaggaa |
| | | | agccaaaacc |
| 4410 | NM_017787 | C10orf26 | ttcaatgacaggcttggactagctgtggcccagacatcggccctgcccagaattgccagg |
| | | | aggaggcttt |
| 4411 | NM_017794 | KIAA1797 | tgacagcttgacacatgcctcctaagagaggagtgcattgctttagtacccgggccagttg |
| | | | agactgaaa |
| 4412 | NM_018136 | ASPM | agagcctctgatgtacgaagtaggtccaaagttgttgaccgtatttacagtctctacaaactt |
| | | | acagctc |
| 4413 | NM_018209 | ARFGAP1 | gagctgtcagcgacgtgaggtgtcccttctcgttgagatatttaactttggttttgctctagttc |
| | | | tttct |
| 4414 | NM_018248 | FLJ10858 | gcaagctaccgactagaaatactataatcagttggacatctagcagggtggatcatgttatg |
| | | | gactccgt |
| 4415 | NM_018462 | C3orf10 | agcccttgaacggagaatagagtacattgaagctcgggtgacaaaaggtgagacactca |
| | | | cctagaacagt |
| 4416 | NM_018664 | SNFT | ctcctgggcaaggggctgttgtgagcccattggtatagcaactagtggtgctgtctactgca |
| | | | aagcaaat |
| 4417 | NM_018951 | HOXA10 | gaggactggggtaagcggaataaactagagaagggagacattgtttggatttcctttatact |
| | | | gtgaagtt |
| 4418 | NM_019028 | ZDHHC13 | ttgtccctcaggaagacaccatacaatcttggattcatgcagaacctggcagatttctttcag |
| | | | tgtggct |
| 4419 | NM_019071 | ING3 | tgcagttcatgagggagggggcgggggactgaaggggaaagggcgttaaagtgataca |
| | | | tttttataccaa |
| 4420 | NM_019102 | HOXA5 | atggagatcatagttccgtgagcgagcaattcagggactcggcgagcatgcactccggc |
| | | | aggtacggcta |
| 4421 | NM_019111 | HLA-DRA | acaaagcgctccaactatacttcgatcaccaatgtacctccagacgtaactgtgctcacaa |
| | | | acagccctg |
| 4422 | NM_019841 | TRPV5 | tgcctatgcaagtgtctaacttctgtgcctgttaatcatgggagggtgagacagaacaatcc |
| | | | ctaaaggg |
| 4423 | NM_019846 | CCL28 | catcaggggaaacacgaaacatacggccataaaactccttattagagagtctacagataa |
| | | | atctacagag |
| 4424 | NM_019857 | CTPS2 | acaacgacccccgcgttttccccgtactagatggttagggcgcatagtgccgaactacgct |
| | | | gctgctaca |
| 4425 | NM_020310 | MNT | ctgcaaatctagtgccgaaatctagtgccgaatgactatgtccagattggtgacgatgggt |
| | | | gttgctgtt |
| 4426 | NM_020368 | SAS10 | gcttagggaaatttcacagttcattgtggagtgttaaacttagaacatgtgtaacttttcacata |
| | | | aagag |
| 4427 | NM_020371 | AVEN | ggctgtccttcaggaccagccagtttacaagcatgtctcaagctagtgtgttccattatgctc |
| | | | acagcag |
| 4428 | NM_020657 | ZNF304 | acaaaaggagattcgaacattctagaggggacatccacaagtctcggtgcagttatgatg |
| | | | gtgtgggatg |
| 4429 | NM_020841 | OSBPL8 | gtgaagcggttggatccattactcaacccttacctagcagttatttgatcatccgagctacttc |
| | | | agagtc |
| 4430 | NM_020843 | ZNF291 | tgtttagtaacaagattaccttcttaatggacctcctgatacaccagttgacggtttatgttcca |
| | | | gatga |
| 4431 | NM_020892 | DTX2 | agcaggtctgcagtgatcagatgggtttctcacatattgttaagttgaaagtagccgtggctc |
| | | | agtatga |
| 4432 | NM_020944 | GBA2 | aggggccatgggggctgtgaatgggatgcagccccatggtgtccctgataaatccagtgt |
| | | | gcagtctgat |
| 4433 | NM_020956 | PRX | cacagctgcccgccgtggaaattgaggaagggcggctggagatgatagagacaaaagt |
| | | | caagccctcttc |
| 4434 | NM_020998 | MST1 | cccctgaatgaacaaagtgtggacatgccaatacacattactgacaggtatgcccacctga |
| | | | cctgcaccc |
| 4435 | NM_021019 | MYL6 | gaaaggcgagcaaccagaacagagcacaacaaaggacgagcagcgccagccgagac |
| | | | gacgtaaggacaca |
| 4436 | NM_021038 | MBNL1 | ttaatgcgaacagacagacttgaggtatgtcgagagtaccaacgtggcaattgcaaccga |
| | | | ggagaaaatg |
| 4437 | NM_021071 | DO | gcatctctctcctttttgaccagtgtcatcatcttttccaaaagcagagtataaagaaatcttgt |
| | | | ggctc |
| 4438 | NM_021103 | TMSB10 | ctgggggtgaaatcttacagcgcgggttccgttacctttcccggatacttttccgggaatgat |
| | | | gtacaca |
| 4439 | NM_021111 | RECK | tacagataattacctactctggctagaagctaggggtcccagtgaagagccactgccatta |
| | | | aagaatatg |
| 4440 | NM_021114 | SPINK2 | tacgctcacccacctcactatactctcccttatcgcgcgtctatatggccccgatcacccttc |
| | | | cgccttg |
| 4441 | NM_021643 | TRIB2 | ccgcccgagactccgaacttgtcgcattgcgtttcttgtatcgggaaatacttattgttggaa |
| | | | cctctgg |
| 4442 | NM_021814 | ELOVL5 | gcagtgtattacttatgtgtttgtggagatgttggtgtaaacctactgcactgctaattctataa |
| | | | aagta |
| 4443 | NM_021906 | USP9X | taacttacatggaaagcatgagactatatgctagagaccatgaagattatgacccacaaac |
| | | | tgtgaggct |
| 4444 | NM_021949 | ATP2B3 | ttgtaaactgttccctgatgtccttgtttaaacgataataagaaaaagggcaactccatttgtt |
| | | | gagggt |
| 4445 | NM_021960 | MCL1 | tctggttaaacagctgtacttttgatagctgtgccaggaagggttaggaccaactacaaatta |
| | | | atgttgg |
| 4446 | NM_022067 | C14orf133 | ggttgtcgaaattttgcacaagaacaatgcccctgtgcagatattacaggagtatgtcaatct |
| | | | ggtggaa |
| 4447 | NM_022127 | SLC28A3 | atcgggtccctaggaatcgtgatcggcggactcacgtccatggctccttccagaaagcgt |
| | | | gatatcgcct |
| 4448 | NM_022161 | BIRC7 | catttctgctgcaaacctggtcagagccagtgttccctccatgggacctaaagacagtgcc |
| | | | aagtgcctg |
| 4449 | NM_022334 | ITGB1BP1 | aattggagtttactgtcatctgtttccgtttctctaagatagggatcctggcgaagcccatgaa |
| | | | agccat |
| 4450 | NM_022366 | TFB2M | tatgatgaaaccctggaagataggtagcaactagactgtcgtttttggtggagcggttcattt |
| | | | atttgga |
| 4451 | NM_022436 | ABCG5 | ccagagttaaatgcagatgtccacacgaggggtcggagttacctgatcacatcgagagag |
| | | | tgctgggcag |
| 4452 | NM_022438 | MAL | aaatcagtgcaatgaagactttggatttattaaaaccaagttgtccctaaggaatcagatgtg |
| | | | ggcatct |
| 4453 | NM_022469 | GREM2 | tagcaatgaaagcaatatgatatgctgcagtgtgaagctccttctggggttatcgtatgtaca |
| | | | aagttta |
| 4454 | NM_022549 | FEZ1 | atgcagattcctggtatcacttgctcgctactttctagggtcttaatagtagtcaagaaaagg |
| | | | gatctga |
| 4455 | NM_022716 | PRRX1 | ggaattgcagttagagagagtaaggaataccatttagtcatctatccgttcttcacttagcag |
| | | | gaatatg |
| 4456 | NM_024006 | VKORC1 | caatgatcagctcccgcgactgctacgtgctcattagtgttcgtccgacgacgaccgtcata |
| | | | tgtccacc |
| 4457 | NM_024319 | C1orf35 | ataccagggatggcccctggcttggcctgcgaaggtgaacctgcccagatttatcagtag |
| | | | aggctggact |
| 4458 | NM_024408 | NOTCH2 | ctgtttgtatgagaaacccagtaaccaataaaatgaccgcatattcctgactaaacgtagta |
| | | | aggaaaat |
| 4459 | NM_024424 | WT1 | ctacaagacactgcccccacgctctctgctcagtcctctcccacacgatctatccctaaacc |
| | | | tttcgcgg |
| 4460 | NM_024713 | C15orf29 | tgcacgtctgtgatttgagaggtgagttatttaagaggccagttttcaggacatgggaatttg |
| | | | aattgta |
| 4461 | NM_024728 | C7orf10 | tccagaatggctctggtattaatgaatctagtgccttttaaatgtatcccacgttttgttccctac |
| | | | catc |
| 4462 | NM_025263 | PRR3 | gcctttctcagtgtccgtggcatttttgtgacttcccagcactagaataagttttcctgccaaa |
| | | | atgagt |
| 4463 | NM_030660 | ATXN3 | acagttgagtcatggaatgtaggtgtctgcttcacatcttttagtaggtatagcttgtcaaaga |
| | | | tggtga |
| 4464 | NM_030674 | SLC38A1 | cagatgggtgatttaagtgagtcacaagtcacaaaactttgctattcatagttaatcaaatag |
| | | | aactggg |
| 4465 | NM_030915 | LBH | tttcagtaaaaatgcctgttgtgagatgaacctcctgtaacttctatctgttcttttttgaggctc |
| | | | aggg |
| 4466 | NM_030926 | ITM2C | cattcgatatgctaaccgttctcagccctgagccttggagaggagggctgtaacgccttca |
| | | | gtcagtctc |
| 4467 | NM_030935 | THG-1 | cccacgatgccagcctggttcacaaatctccagaccccttcggagcagtagcagctcaga |
| | | | agttcagcct |
| 4468 | NM_031243 | HNRPA2B1 | gacagtttcttagctttttaattgttgtttctttctagtggtctttgtaagagtgtagaagcattcct |
| | | | tc |
| 4469 | NM_031423 | CDCA1 | gagactgctgatattctatgtccaaaagcaaaacggacaagtcggtttttaagtggcattatc |
| | | | aacttta |
| 4470 | NM_031966 | CCNB1 | ccttttggtttacctggggatccaattgatgtatatgtttatatactgggttcttgttttatatacct |
| | | | gg |
| 4471 | NM_032121 | DKFZp564K142 | atgaatggagacaagttccgtcgccttgtgaaagccccaccgagaaattactccgttatcgt |
| | | | catgttca |
| 4472 | NM_033238 | PML | acagaaactgatgttccatgcatgttcctgcttccaaagcctgaccttccaaagcttgcttcc |
| | | | ttcctcc |
| 4473 | NM_033512 | TSPYL5 | ggatgcctagttgaggatgttcccaaagttttgtccaatcttatcattagtagattttataagcc |
| | | | acaga |
| 4474 | NM_033554 | HLA-DPA1 | ccccacactcgcgagaacgaccaaaaatgaaacgaatacggccggaaaccgatcccat |
| | | | gtgccgggccaa |
| 4475 | NM_033642 | FGF13 | ccaaacaaacaggcagagttcactattctatctgccattagaccttcttatcatccatactaaa |
| | | | gcccca |
| 4476 | NM_057168 | WNT16 | ggataagatccttgaatatggaacttagttacaggactcaataatggtgggtgaacattagt |
| | | | cattttta |
| 4477 | NM_058195 | CDKN2A | gctcttctgttcctgcgacacctctgttctatgatcttcgctcttgttgtttctcaccatgtgtcag |
| | | | ctg |
| 4478 | NM_080284 | ABCA6 | acgacaggagtctagttccactgtggaattcctttctggttgtacatttgctaggtagtgacaa |
| | | | ctaatg |
| 4479 | NM_130439 | MXI1 | cctcactctttatagtgcacaaaatgaatgaggtctgggctaggtagaaaaagggtcaatg |
| | | | ctatttttg |
| 4480 | NM_133259 | LRPPRC | atttccaatcagtttgattgggctctaatgagactagatctttctgttcgaagaactggccgca |
| | | | ttccaa |
| 4481 | NM_133378 | TTN | cagtactacctgggttgagttagcaaccaccgttatacgtactacctataaagccacccgc |
| | | | cttactact |
| 4482 | NM_138714 | NFAT5 | tccagtaaaaaactcctgcactgaagtcattgtgacttgagtagttacagactgattccagtg |
| | | | aacttga |
| 4483 | NM_144578 | C14orf32 | cctggataaaggcactttcactgcctgtcactgatcagcagatactgacttgttgccattaag |
| | | | tgaactt |
| 4484 | NM_144628 | TBC1D20 | actaatcacctcttctgtcattccgtccttggccaccgctcagtgggaatggtctctgatctg |
| | | | gatgctc |
| 4485 | NM_145698 | ACBD5 | ttacaagcagtctgggataactaactcctagggataaccatttctatcctgttccaaagtctta |
| | | | ttttat |
| 4486 | NM_147180 | PPP3R2 | gtggcaatgaccttgaactgagagcctgtatctggatttagcacttgaaagatctaactgga |
| | | | tatttggg |
| 4487 | NM_152739 | HOXA9 | ttgaatattgtttttgcaccagacgaacagtgaggaaattcggagctatacatatgtgcagaa |
| | | | ggttact |
| 4488 | NM_152788 | EB-1 | ggttgatgtatttagcttgaatccttcactttagtggtctaatcaacagataaatgacaagcaa |
| | | | agaatg |
| 4489 | NM_152827 | SNX3 | agaaagtatcttcctccaggcttgtaatacccttcacatggaagattaatgagggaaatcttt |
| | | | atattct |
| 4490 | NM_152890 | COL24A1 | acccagcacgaatctgcaaagatttacttaactgtgaacaaaaagtatcagatggaaaata |
| | | | ctggattga |
| 4491 | NM_173156 | C1orf16 | agtctcccagcctccactttcagagtgaaattcaaggcagcacggacatgtgcccatcag |
| | | | gcacagaaga |
| 4492 | NM_173216 | SIAT1 | ggtggcattccgacagcaggacatacatgttggtgtgaagactgggacgacactgggtag |
| | | | aatctagttt |
| 4493 | NM_173609 | C15orf21 | actgagatagaaggaccagccactttgaacgccttagaggcaaaaagagctttgtgagtt |
| | | | gtaagaggaa |
| 4494 | NM_173852 | KRTCAP2 | agagagccccggatttctcctggactccatgtgcagatgcctagtatttccattctgaccgtt |
| | | | gccctcc |
| 4495 | NM_175739 | SERPINA9 | ctgagaaagtggagccactcactccagaaaaggtggatagaggtgttcatccccagatttt |
| | | | ccatttctg |
| 4496 | NM_175744 | RHOC | ttgttttcggcaggggccttgtctctcactgcatttggtcaggggggcatgaataaaggcta |
| | | | caggctcc |
| 4497 | NM_178586 | PPP2R5C | ctgtacatcagaaatgtcactattccaagtgtctttttagtgtggctttagtatggcttccttttaa |
| | | | tat |
| 4498 | NM_181311 | TAZ | accagggaaagtgaacatgagttccgaattcctgcgtttcaagtggggaatcgggcgcct |
| | | | gattgctgag |
| 4499 | NM_181336 | LEMD2 | atgtctggatgatcggcagggcaggaatctgagcgctggccccgtggtgaggccatgttc |
| | | | tcataatcag |
| 4500 | NM_181339 | IL24 | cctaggcaaagagtatatgtaggaggtgggatatcacttccatgacataagtgctattgcag |
| | | | agccgtgg |
| 4501 | NM_181430 | FOXK2 | tggtatgtttgtatttggggtgtccctccggctctaggcggcctctgacctgctgtctactccc |
| | | | cacctt |
| 4502 | NM_181493 | ITPA | tggcttctgctgtcgtgtccatccttccttcgtctggctcgtgattcacttattcgccctccctac |
| | | | tttg |
| 4503 | NM_181702 | GEM | tgtggtttcccccattgtagcagggagctagcgtattagccttgtgggcaacatgatgcatg |
| | | | ggaaatga |
| 4504 | NM_182729 | TXNRD1 | gctggtctaagcaagctgagatcatttgcaatggaaaacacgtaacttgtttaaaagtttttct |
| | | | ggtagc |
| 4505 | NM_182810 | ATF4 | aaaagggtcccctagttgaggatagtcaggagcgtcaatgtgcttgtacatagagtgctgt |
| | | | agctgtgtg |
| 4506 | NM_183395 | CIAS1 | agacaccaggacaatgacagcatcgggtgttgttgtcatcacagcgcctcagttagagga |
| | | | tgttcctctt |
| 4507 | NM_198232 | RNASE1 | ccttgtccgggcgcaacatgcaattatattaaactcggccaaaccctctgcgggataaagg |
| | | | ggttttgtt |
| 4508 | NM_198400 | NEDD4 | ggatgattctgtgactcatagaacatgatgttaaatgagaccagactcaactgccaccagtc |
| | | | cccagctg |
| 4509 | NM_199335 | FYB | gaccccgaagatggtgcagcttggatagtgaatgtgcaagttagctatgcagagtgtactt |
| | | | ccttttctg |
| 4510 | NM_199423 | WWP2 | ctcactttgaaagtggtgtccgcaaagcccaaggtgcataatcgtcaacctcgaattaactc |
| | | | ctacgtgg |
| 4511 | NM_201592 | GPM6A | tagtaggtagggggtactactagggatatctgtggcatgattatgcattccgtagtattattta |
| | | | attaat |
| 4512 | NM_201632 | TCF7 | agcggcatgtacaaagagaccgtctactccgccttcaatctgctcatgcattacccacccc |
| | | | cctcgggag |
| 4513 | NM_201998 | SF1 | aaagttgccgagggtaggttcatctccaggtttcgggattcccatccgtcctggcgatcctg |
| | | | ccagcagt |
| 4514 | NM_212474 | FN1 | ttgttaccgtgggcaactctgtcaacgaaggcttgaaccaacctacggatgactcgtgcttt |
| | | | gaccccta |
| 4515 | R14777 | CYFIP2 | tcctttcacccaagaaccacaacgagacaaacctgccaacgtccagccttattacctctatg |
| | | | gatccaag |
| 4516 | R56397 | PIK3C3 | cgtaaacagtgttacacggctttcctccacctgcgaaggtattctaatctgattttgaacttgtt |
| | | | ttcct |
| 4517 | R59027 | GRP58 | ctcctccagcctcattgaaactctttttgtggatttgatgaccttatggatattgccaaagtttaa |
| | | | ggca |
| 4518 | R72151 | GNL3 | gcagactgctaaactgttctctgtataagttatggtatgcatgagctgtgtaaattttgtgaata |
| | | | tgtat |
| 4519 | R79128 | MAP3K1 | aagtcttactctgtatgtaacaatccatcattcaccttcactactggtagtaacatagagctgc |
| | | | catttt |
| 4520 | R86893 | C6orf110 | tgcacagttctcgaagcccgcccgtgttacaacgtggctcgcctaatgttcctcgatgcag |
| | | | agaggaaga |
| 4521 | T07281 | NRIP1 | gtgagaaagggcatacatccacagattcactttgtttatgcatatgtagatacaaggatgca |
| | | | catataca |
| 4522 | T28925 | ITGAL | tttccctgtttaatgattgacgtacttagcagctatctctcagtgaactgtgagggtaaaggct |
| | | | atactt |
| 4523 | U60115 | FHL1 | aatacatactaacattcttgtaggagtggttagagaagctgatgcctcatttctacattctgtc |
| | | | attagc |
| 4524 | U79271 | AKT3 | acattcatctggtttagacttaatatgccacaacgcaccacgaccttcccagggtgacacc |
| | | | gcctcagcc |
| 4525 | W04885 | MYL4 | cagtacatccccgcactgggcgtaggtgattttcatctctccagtcggggtccggtcaaac |
| | | | aatgaaaag |
| 4526 | X66087 | MYBL1 | acttagggatggctcattgaacgatggtggtaatatggcgctaaaacatacaccactgaaa |
| | | | acactacca |
| 4527 | AK024272 | | gtgatgttataaatgaggggagtaaaggaacatgagataaccactaatatgacataccaat |
| | | | agactatga |
| 4528 | AK025231 | IGLC2 | agcaacaggcggcgagtcaaaacccaaggtgtgtttatctaaactgggcaattcctcttcta |
| | | | ggaattta |
| 4529 | AL080190 | | taaagcgaggaagcagatgccaggcagtgaagacttgcacaccgtcacactgttaactg |
| | | | gtagagaggca |
| 4530 | BU618233 | | cgataaatccctttaatctcctctacatatactgagtattaaaaaacagaatcggctagaaca |
| | | | ttgttgc |

The polynucleotide probes of an HCP combination can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific examples of polynucleotide probes useful in this invention include oligonucleotides containing modified backbones or non-natural internucleoside linkages. As defined in this specification, oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary polynucleotide probes having modified oligonucleotide backbones include, for example, those with one or more modified internucleotide linkages that are phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkyl-phosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Exemplary modified oligonucleotide backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulfide, sulfoxide and sulphone backbones; formacetyl and thioformacetyl backbones; methylene formacetyl and thioformacetyl backbones; alkene containing backbones; sulphamate backbones; methyleneimino and methylenehydrazino backbones; sulphonate and sulfonamide backbones; amide backbones; and others having mixed N, O, S and CH₂ component parts.

The present invention also contemplates oligonucleotide mimetics in which both the sugar and the internucleoside linkage of the nucleotide units are replaced with novel groups. The base units are maintained for hybridization with an appropriate nucleic acid target compound. An example of such an oligonucleotide mimetic, which has been shown to have excellent hybridization properties, is a peptide nucleic acid (PNA) [Nielsen et al., Science, 254:1497-1500 (1991)]. In PNA compounds, the sugar-backbone of an oligonucleotide is replaced with an amide containing backbone, in particular an aminoethylglycine backbone. The nucleobases are retained and are bound directly or indirectly to aza-nitrogen atoms of the amide portion of the backbone.

The present invention also contemplates polynucleotide probes comprising “locked nucleic acids” (LNAs), which are novel conformationally restricted oligonucleotide analogues containing a methylene bridge that connects the 2′-O of ribose with the 4′-C (see, Singh et al., Chem. Commun., 1998, 4:455-456). LNA and LNA analogues display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described (see Koshkin et al., Tetrahedron, 1998, 54:3607-3630). Studies of mismatched sequences show that LNA obey the Watson-Crick base pairing rules with generally improved selectivity compared to the corresponding unmodified reference strands.

LNAs form duplexes with complementary DNA or RNA or with complementary LNA, with high thermal affinities. The universality of LNA-mediated hybridization has been emphasized by the formation of exceedingly stable LNA:LNA duplexes (Koshkin et al., J. Am. Chem. Soc., 1998, 120:13252-13253). LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA (Singh et al., J. Org. Chem., 1998, 63, 10035-10039) and 2′-methylamino-LNA has been described and thermal stability of their duplexes with complementary RNA and DNA strands reported. Preparation of phosphorothioate-LNA and 2′-thio-LNA have also been described (Kumar et al., Bioorg. Med. Chem. Lett., 1998, 8:2219-2222).

Modified polynucleotide probes may also contain one or more substituted sugar moieties. For example, oligonucleotides may comprise sugars with one of the following substituents at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Examples of such groups are: O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-methoxyethoxy (2′-O—CH₂CH₂OCH₃, also known as 2′-O-(2-methoxyethyl) or 2′-MOE) [Martin et al., Helv. Chim. Acta, 78:486-504 (1995)], 2′-dimethylaminooxyethoxy (O(CH₂)₂ON(CH₃)₂ group, also known as 2′-DMAOE), 2′-methoxy (2′-O—CH₃), 2′-aminopropoxy (2′-OCH₂CH₂CH₂NH₂) and 2′-fluoro (2′-F).

Similar modifications may also be made at other positions on the polynucleotide probes, particularly the 3′ position of the sugar on the 3′ terminal nucleotide or in 2′-5′ linked oligonucleotides and the 5′ position of 5′ terminal nucleotide. Polynucleotide probes may also have sugar mimetics such as cyclobutyl moieties in place of the pentofuranosyl sugar.

Polynucleotide probes may also include modifications or substitutions to the nucleobase. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. I., ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the polynucleotide probes of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi, Y. S., (1993) Antisense Research and Applications, pp 276-278, Crooke, S. T. and Lebleu, B., ed., CRC Press, Boca Raton].

One skilled in the art will recognise that it is not necessary for all positions in a given polynucleotide probe to be uniformly modified. The present invention, therefore, contemplates the incorporation of more than one of the aforementioned modifications into a single polynucleotide probe or even at a single nucleoside within the probe.

One skilled in the art will also appreciate that the nucleotide sequence of the entire length of the polynucleotide probe does not need to be derived from the target gene. Thus, the polynucleotide probe may comprise nucleotide sequences at the 5′ and/or 3′ termini that are not derived from the target gene. Nucleotide sequences which are not derived from the nucleotide sequence of the target gene may provide additional functionality to the polynucleotide probe. For example, they may provide a restriction enzyme recognition sequence or a “tag” that facilitates detection, isolation, purification or immobilisation onto a solid support. Alternatively, the additional nucleotides may provide a self-complementary sequence that allows the primer/probe to adopt a hairpin configuration. Such configurations are necessary for certain probes, for example, molecular beacon and Scorpion probes, which can be used in solution hybridization techniques.

The polynucleotide probes can incorporate moieties useful in detection, isolation, purification, or immobilisation, if desired. Such moieties are well-known in the art (see, for example, Ausubel et al., (1997 & updates) Current Protocols in Molecular Biology, Wiley & Sons, New York) and are chosen such that the ability of the probe to hybridize with its target sequence is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

### 1.2.1 Preparation of Polynucleotide Probes

The polynucleotide probes of the present invention can be prepared by conventional techniques well-known to those skilled in the art. For example, the polynucleotide probes can be prepared using solid-phase synthesis using commercially available equipment, such as the equipment available from Applied Biosystems Canada Inc., Mississauga, Canada. As is well-known in the art, modified oligonucleotides, such as phosphorothioates and alkylated derivatives, can also be readily prepared by similar methods. The polynucleotide probes can also be synthesized directly on a solid support according to methods standard in the art. This method of synthesizing polynucleotides is particularly useful when the polynucleotide probes are part of a nucleic acid array.

Alternatively, the polynucleotide probes of the present invention can be prepared by enzymatic digestion of the naturally occurring target gene, or mRNA or cDNA derived therefrom, by methods known in the art.

### 1.2.2 Testing of Polynucleotide Probes

Each polynucleotide probe suitable for use in the HCP combination must be able to specifically detect the expression of a target gene in the HCP set. As noted previously, the specificity or uniqueness of the polynucleotide probe can be determined in silico using methods known in the art.

Alternatively, the ability of the polynucleotide probes to specifically detect the expression of the target gene or mRNA in a sample can be assessed by other standard methods (see, for example, Ausubel et al., (1997 & updates) Current Protocols in Molecular Biology, Wiley & Sons, New York), including hybridization techniques such as Southern or Northern blotting using appropriate controls, and may include one or more additional steps, such as reverse transcription, transcription, PCR, RT-PCR and the like. The testing of the specificity of the polynucleotide probes of the HCP combination using these methods is well within the abilities of a worker skilled in the art.

### 1.3 Hematological Cancer Profiling (HCP) Combination

An HCP combination comprises a plurality of polynucleotide probes designed to target genes of one or more HCP set, as described above. The HCP combination can be tailored by selection of polynucleotide probes that correspond to those HCP sets that represent a hematological cancer and/or a feature(s) of interest in a hematological cancer. Thus, for example, if one is interested in investigating various features of a single hematological cancer, for example, lymphoma, one would select an HCP combination that comprised probes to the HCP set representing the lymphoma of interest. Similarly, if one is interested in investigating features of several lymphomas, then a combination would be selected that comprised probes representing several HCP sets.

In one embodiment of the present invention, an HCP combination comprises a plurality of polynucleotide probes derived from the nucleotide sequences of the genes of one HCP set. In another embodiment, an HCP combination comprises a plurality of polynucleotide probes derived from the nucleotide sequences of the genes of two or more HCP sets. In another embodiment, the HCP combination comprises a plurality of polynucleotide probes derived from the nucleotide sequences of the genes of three or more HCP sets. In a further embodiment, the HCP combination comprises a plurality of polynucleotide probes derived from the nucleotide sequences of the genes of four or more HCP sets. In other embodiments, the HCP combination comprises a plurality of polynucleotide probes derived from the nucleotide sequences of five or more HCP sets, and six or more HCP sets.

Members of an HCP set may be targeted by one or more than one polynucleotide probe of the HCP combination. Therefore, the HCP combination can comprise several polynucleotide probes targeted to only one gene in an HCP set and other probes that each target a different gene. In one embodiment, polynucleotide probes within the HCP combination each target a different member of an HCP set. In another embodiment, one or more polynucleotide probes of the HCP combination target the same member of an HCP set.

Accordingly, the HCP combination comprises between one and about 10,000 polynucleotide probes. In one embodiment of the present invention, the HCP combination comprises at least 2 polynucleotide probes. In another embodiment, the HCP combination comprises at least 5 polynucleotide probes. In other embodiments, the HCP combination comprises at least 10, 20, 30, 40, 50, 100, 150, 200 and 300 polynucleotide probes. In one embodiment, the HCP combination comprises from about 10 to about 300 polynucleotide probes. In another embodiment, the HCP combination comprises from about 20 to about 300 polynucleotide probes. In a further embodiment, the HCP combination comprises from about 30 to about 300 polynucleotide probes. In other embodiments, the HCP combination comprises from about 40 to about 300, from about 50 to about 300, from about 75 to about 300, and from about 100 to about 300 polynucleotide probes.

In an alternate embodiment, the HCP combination comprises from about 100 to about 10,000 polynucleotide probes. In a further embodiment, the HCP combination comprises from about 200 to about 5,000 polynucleotide probes. In another embodiment, the HCP combination comprises from about 200 to about 4,000 polynucleotide probes. In yet another embodiment, the HCP combination comprises from about 200 to about 3,000 polynucleotide probes. In other embodiments, the HCP combination comprises from about 200 to about 2,000, from about 300 to about 2,000, from about 400 to about 2,000, from about 500 to about 2,000, from about 500 to about 1,500, from about 750 to about 1,500, from about 750 to about 1250, and from about 800 to about 1,200 polynucleotide probes.

In a further embodiment, the HCP combination comprises from about 1,000 to about 10,000 polynucleotide probes. For example, the HCP combination can comprise from about 2,000 to about 10,000 polynucleotide probes. In one embodiment, the HCP combination comprises from about 2,500 to about 9,000 polynucleotide probes. In yet another embodiment, the HCP combination comprises from about 3,000 to about 8,000 polynucleotide probes. In other embodiments, the HCP combination comprises from about 3,000 to about 7,000, from about 3,000 to about 6,000, from about 3,500 to 6,000, from about 4,000 to about 6,000, and from about 4,000 to about 5,000 polynucleotide probes.

As indicated above, an HCP combination comprises a plurality of polynucleotide probes designed to target genes of one or more HCP set. Representative, non-limiting examples of candidate genes for inclusion in HCP sets are shown in Table 1, above. Accordingly, in one embodiment of the present invention, the HCP combination comprises between ten and 5,000 polynucleotide probes, wherein each of the probes comprise a sequence corresponding to or complementary to, the sequence of one of the genes listed in Table 1. Representative, non-limiting examples of HCP sets are provided in Tables 2-19. Thus, in another embodiment, the HCP combination comprises between ten and 5,000 polynucleotide probes, wherein each of the probes comprises a sequence corresponding to or complementary to a gene of an HCP set selected from the group of:
(a) an HCP set comprising one or more genes as set forth in Table 2;
(b) an HCP set comprising one or more genes as set forth in Table 3;
(c) an HCP set comprising one or more genes as set forth in Table 4;
(d) an HCP set comprising one or more genes as set forth in Table 5;
(e) an HCP set comprising one or more genes as set forth in Table 6;
(f) an HCP set comprising one or more genes as set forth in Table 7;
(g) an HCP set comprising one or more genes as set forth in Table 8;
(h) an HCP set comprising one or more genes as set forth in Table 9;
(i) an HCP set comprising one or more genes as set forth in Table 10;
(j) an HCP set comprising one or more genes as set forth in Table 11;
(k) an HCP set comprising one or more genes as set forth in Table 12;
(l) an HCP set comprising one or more genes as set forth in Table 13;
(m) an HCP set comprising one or more genes as set forth in Table 14;
(n) an HCP set comprising one or more genes as set forth in Table 15;
(o) an HCP set comprising one or more genes as set forth in Table 16;
(p) an HCP set comprising one or more genes as set forth in Table 17;
(q) an HCP set comprising one or more genes as set forth in Table 18;
(r) an HCP set comprising one or more genes as set forth in Table 19; and
(s) an HCP set comprising one or more genes selected from the genes set forth in Tables 2-19.

In a further embodiment, the HCP combination comprises between ten and 5,000 polynucleotide probes, wherein each of the probes comprises a sequence corresponding to or complementary to a gene of an HCP set selected from the group of:
(a) an HCP set as set forth in Table 2;
(b) an HCP set as set forth in Table 3;
(c) an HCP set as set forth in Table 4;
(d) an HCP set as set forth in Table 5;
(e) an HCP set as set forth in Table 6;
(f) an HCP set as set forth in Table 7;
(g) an HCP set as set forth in Table 8;
(h) an HCP set as set forth in Table 9;
(i) an HCP set as set forth in Table 10;
(j) an HCP set as set forth in Table 11;
(k) an HCP set as set forth in Table 12;
(l) an HCP set as set forth in Table 13;
(m) an HCP set as set forth in Table 14;
(n) an HCP set as set forth in Table 15;
(o) an HCP set as set forth in Table 16;
(p) an HCP set as set forth in Table 17;
(q) an HCP set as set forth in Table 18; and
(r) an HCP set as set forth in Table 19.

In another embodiment, the HCP combination represents more than one HCP set and comprises between about 10 and about 5,000 probes, each of said probes comprising a sequence corresponding to, or complementary to, a gene listed in any one of Tables 2-19.

In a further embodiment, the HCP combination comprises between ten and 5,000 polynucleotide probes, each of the probes having a sequence corresponding to or complementary to a nucleotide sequence selected from any one of Tables 20-23, wherein the HCP combination represents one or more HCP sets selected from the group of:
(a) an HCP set as set forth in Table 2;
(b) an HCP set as set forth in Table 3;
(c) an HCP set as set forth in Table 4;
(d) an HCP set as set forth in Table 5;
(e) an HCP set as set forth in Table 6;
(f) an HCP set as set forth in Table 7;
(g) an HCP set as set forth in Table 8;
(h) an HCP set as set forth in Table 9;
(i) an HCP set as set forth in Table 10;
(j) an HCP set as set forth in Table 11;
(k) an HCP set as set forth in Table 12;
(l) an HCP set as set forth in Table 13;
(m) an HCP set as set forth in Table 14;
(n) an HCP set as set forth in Table 15;
(o) an HCP set as set forth in Table 16;
(p) an HCP set as set forth in Table 17;
(q) an HCP set as set forth in Table 18; and
(r) an HCP set as set forth in Table 19.

Representative, non-limiting examples of suitable probe sequences targeted to the genes of the HCP sets in Tables 2-19 are provided in Tables 20-23. Accordingly, in a specific embodiment of the present invention, the HCP combination comprises at least ten polynucleotide probes, wherein each of the probes comprises at least 15 consecutive nucleotides of one of the sequences set forth in SEQ ID NOs:1-4530 (Tables 20-23). In another embodiment, the HCP combination comprises at least 20 polynucleotide probes, wherein each of the probes comprises at least 15 consecutive nucleotides of one of the sequences set forth in SEQ ID NOs:1-4530 (Tables 20-23). In another embodiment, the HCP combination comprises at least 30 polynucleotide probes, wherein each of the probes comprises at least 15 consecutive nucleotides of one of the sequences as set forth in SEQ ID NOs:1-4530 (Tables 20-23). In a further embodiment, the HCP combination comprises at least 40 polynucleotide probes, wherein each of the probes comprises at least 15 consecutive nucleotides of one of the sequences as set forth in SEQ ID NOs:1-4530 (Tables 20-23). In other embodiments, the HCP combination comprises at least 20, at least 30, at least 40, at least 50, at least 60, at least 70, at least 80, at least 90 and at least 100 polynucleotide probes, wherein each of the probes comprises at least 15 consecutive nucleotides of one of the sequences as set forth in SEQ ID NOs:1-4530 (Tables 20-23). In further embodiments, the HCP combination comprises at least 20, at least 30, at least 40, at least 50, at least 60, at least 70, at least 80, at least 90 and at least 100 polynucleotide probes, wherein each of the probes comprises at least 15 consecutive nucleotides of one of the sequences as set forth in SEQ ID NOs:1-1153 (Table 20). In other embodiments, the HCP combination comprises at least 20, at least 30, at least 40, at least 50, at least 60, at least 70, at least 80, at least 90 and at least 100 polynucleotide probes, wherein each of the probes comprises at least 15 consecutive nucleotides of one of the sequences as set forth in SEQ ID NOs:1154-2299 (Table 21). In further embodiments, the HCP combination comprises at least 20, at least 30, at least 40, at least 50, at least 60, at least 70, at least 80, at least 90 and at least 100 polynucleotide probes, wherein each of the probes comprises at least 15 consecutive nucleotides of one of the sequences as set forth in SEQ ID NOs:2300-3426 (Table 22). In further embodiments, the HCP combination comprises at least 20, at least 30, at least 40, at least 50, at least 60, at least 70, at least 80, at least 90 and at least 100 polynucleotide probes, wherein each of the probes comprises at least 15 consecutive nucleotides of one of the sequences as set forth in SEQ ID NOs:3427-4530 (Table 23).

### 1.3.1 Testing of the HCP Combination

The HCP combination can be tested for its ability to detect the expression pattern of genes in the one or more HCP sets that it represents using methods well known in the art and one or more appropriate biological samples that represent the hematological cancer and features that are to be investigated with the HCP combination. Suitable biological samples include blood or tissue samples from patients with the hematological cancer, where each sample is known to exhibit one or more feature of a particular hematological cancer. Alternatively, or in addition, biological samples can be obtained from cultures of appropriate hematological cancer cell lines, where each cell line is known to exhibit one or more feature of a particular hematological cancer. Exemplary hematological cancer cell lines that can be used for testing the HCP combination are provided in Table 24, below. One skilled in the art will appreciate that other hematological cancer cell lines are available that are also suitable for testing an HCP combination. Selection of appropriate cell lines for the testing of a particular HCP combination is within the ordinary skills of a worker in the art. If necessary one or more control samples can be used for comparison purposes, for example, a biological sample taken from a healthy subject, or a normal cell line.

The ability of the HCP combination to detect expression patterns in one or more biological samples can be determined using methods known in the art for the analysis of gene expression (see, for example, Ausubel et al., (1997 & updates) Current Protocols in Molecular Biology, Wiley & Sons, New York). Such methods are typically hybridization-based methods, such as Northern blotting. For example, RNA can be prepared from blood or tissue samples from patients or cultures of cell lines, as noted above, and separated on a gel. Probes of the HCP combination can be labelled and used to detect the expression of specific mRNAs from the sample on the gel, according to methods well known in the art. Other testing methods can include additional steps, such as reverse transcription, RT-PCR and/or PCR (including multiplex PCR). Array based methods can also be used to test an HCP combination. The expression pattern detected with the probes of the HCP combination should correspond to the expression pattern expected for each sample.

TABLE 24
Cell lines exhibiting a gene expression pattern representative of a type
of lymphoma or a type of leukemia

| | Representative |
|---|---|
| Type of Lymphoma | cell line |
| anaplastic large-cell lymphoma | Karpas 299 |
| anaplastic large-cell lymphoma | Ki-JK |
| anaplastic large-cell lymphoma | SR 786 |
| anaplastic large-cell lymphoma | SUDHL-1 |
| anaplastic large cell lymphoma | DEL |
| body-cavity lymphoma | BC-1 |
| body-cavity lymphoma | BC-2 |
| body-cavity lymphoma | BC-3 |
| body-cavity lymphoma | BCBL1 |
| Burkitt lymphoma | BL60 |
| Burkitt lymphoma | CA46 |
| Burkitt lymphoma | Namalwa |
| Burkitt lymphoma | Raji |
| Burkitt Lymphoma | Ramos |
| Burkitt lymphoma | Thomas |
| diffuse large B-cell lymphoma | HBL-2 |
| diffuse large B-cell lymphoma | KIS-1 |
| Follicular Lymphoma | FL-18 |
| Follicular Lymphoma | FL-618 |
| Hodgkin's Disease - H/RS derived cell | KMH2 |
| lines | |
| Hodgkin's lymphoma | DEV |
| Hodgkin's lymphoma | HDLM-2 |
| Hodgkin's lymphoma | L428 |
| Hodgkin's lymphoma | L540 |
| immunoblastic lymphoma line | DOHH-2 |
| lymphoblastoid cell lines | GM 03299 |
| lymphoblastoid cell lines | GM 11854 |
| mantle-cell lymphoma | Granta 519 |
| mantle-cell lymphoma | NCEB |
| Peripheral T-cell lymphoma | H9 |
| Peripheral T-cell lymphoma | yt (cytotoxic T-cell |
| | lymphoma |
| T-lymphoblastic leukemia/lymphoma | CEM |
| T-lymphoblastic leukemia/lymphoma | HSB |
| T-lymphoblastic leukemia/lymphoma | Jurkat |
| T-lymphoblastic leukemia/lymphoma | Molt-4 |
| transformed follicular lymphoma | SUDHL-10 |
| transformed follicular lymphoma | SUDHL-4 |
| transformed follicular lymphoma | SUDHL-5 |
| transformed follicular lymphoma | SUDHL-6 |
| Chronic lymphocytic leukemia | MEC-1 |
| Acute myelogenous leukemia | K-562 |

### 2. Hematological Cancer Profiling (HCP) Arrays

The present invention contemplates that the HCP combinations may be provided as an array (HCP array). In the context of the present invention, an “array” is a spatially or logically organized collection of polynucleotide probes. Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that will be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present invention, the HCP array is a chip.

The HCP array can comprise a single representation of each polynucleotide probe, for example, in the form of a spot deposited on a solid surface, or the array can comprise multiple representations of the same polynucleotide probe. Currently available methods of creating chip arrays, for example, allow for the incorporation of up to about 40,000 spots on a single chip.

The HCP arrays of the present invention, therefore, can comprise as few as two spots or as many as 40,000 spots. Typically an array will comprise between about 15 and about 40,000 spots. The actual number of spots included on the array will be dependent on the number of probes in the HCP combination being used to create the array, how many times each probe is represented in the array, the number of control probes, if any, being included in the array, and the format of the array.

As is known in the art probes of varying lengths can be incorporated into the HCP arrays. In one embodiment of the invention, the probes incorporated into the array are between about 20 and about 100 nucleotides in length. In another embodiment of the present invention, the probes incorporated into the array are between about 25 and about 40 nucleotides in length. In another embodiment, the probes are between about 28 and about 32 nucleotides in length. In a further embodiment, the probes incorporated into the array are 30-mers. In alternate embodiments, the probes incorporated into the array are between about 40 and about 55 nucleotides in length, or are between about 48 and about 52 nucleotides in length. In a further embodiment, the probes incorporated into the array are 50-mers. In further embodiments, the probes incorporated into the array are between about 55 and about 65 nucleotides in length, or are between about 58 and about 62 nucleotides in length. In yet another embodiment, the probes incorporated into the array are 60-mers. In other embodiments, the probes incorporated into the array are between about 65 and about 75 nucleotides in length, or are between about 68 and about 72 nucleotides in length. In a further embodiment, the probes incorporated into the array are 70-mers.

HCP arrays can be designed in various formats, for example, in “small” or “large” format. Small arrays comprise polynucleotide probes that are generally representative of less than 500 genes. In one embodiment, the small arrays contemplated by the present invention comprise polynucleotide probes representative of between about 15 and about 499 genes. In another embodiment, a small array comprises polynucleotide probes representative of between about 50 and about 400 genes. In a further embodiment, a small array comprises polynucleotide probes representative of between about 100 and about 350 genes. In yet another embodiment, a small array comprises polynucleotide probes representative of between about 200 and about 300 genes. As indicated above, the probes representing each gene in a small array can be spotted singly or in multiplicate.

Alternatively, HCP arrays can be designed in a “large” format. Large arrays comprise polynucleotide probes that are generally representative of 500 or more genes. In one embodiment, the large arrays contemplated by the present invention comprise polynucleotide probes representative of between about 500 and about 6000 genes. In another embodiment, a large array comprises polynucleotide probes representative of between about 600 and about 4000 genes. In a further embodiment, a large array comprises comprise polynucleotide probes representative of between about 700 and about 2000 genes. In yet another embodiment, a large array comprises polynucleotide probes representative of between about 900 and about 1000 genes. In still another embodiment, the large array comprises polynucleotide probes representative of between about 1000 and about 1300 genes As for the small arrays, the probes representing each gene in a large array can be spotted singly or in multiplicate.

One skilled in the art will appreciate that as the technology for creating arrays advances, it may be possible to include larger numbers of probes in a single array. Arrays created using such technology with the HCP combinations of the present invention are considered to be within the scope of the invention.

Representative non-limiting examples of combinations of probes that can be used in the preparation of an HCP array include combinations that comprise at least 10 of the polynucleotide probes as set forth in any one of Tables, 20, 21, 22, 23, 25, 26, 27, and 28. In one embodiment of the invention, the HCP array comprises at least ten polynucleotide probes, wherein each of the probes comprises at least 15 consecutive nucleotides of one of the sequences as set forth in Table 20, 21, 22, 23, 25, 26, 27, and 28. In other embodiments, the HCP array comprises at least 20, at least 30, at least 40, at least 50, at least 60, at least 70, at least 80, at least 90 and at least 100 polynucleotide probes, wherein each of the probes comprises at least 15 consecutive nucleotides of one of the sequences as set forth in Table 20, 21, 22, 23, 25, 26, 27, and 28.

In another embodiment, the HCP array comprises at least 100 polynucleotide probes, wherein each of the probes comprises at least 15 consecutive nucleotides of one of the sequences as set forth in Table 20, 21, 22, 23, 25, 26, 27, and 28. In other embodiments, the HCP array comprises at least 200, at least 300, at least 400, at least 500, at least 600, at least 700, at least 800 and at least 900 polynucleotide probes, wherein each of the probes comprises at least 15 consecutive nucleotides of one of the sequences as set forth in Table 20, 21, 22, 23, 25, 26, 27, and 28.

In another embodiment, an HCP array comprises a combination of probes as set forth in any one of Tables 20, 21, 22, 23, 25, 26, 27, and 28.

TABLE 25

Polynucleotide probe sequences for preparation
of a small 50 mer nucleic acid array

| GenBank | |
|---|---|
| Accession | |
| Number of | |
| Target gene | Oligonucleotide sequence (5′ to 3′) |
| NM_001728 | TCGTGAGTTCCTCGCAGGGCCGGTCAGAGCTACACATTGAGAACCTGAAC |
| NM_018462 | GCTCGGGTGACAAAAGGTGAGACACTCACTAGAACAGTGCCGTGCTGCTG |
| NM_002266 | CCAGAAACTACCTCTGAAGGCTACACTTTCCAAGTTCAGGATGGGGCTCC |
| NM_004310 | GGCGACTCTGCTGTGGGGAAAACCTCTCTGTTGGTGCGCTTCACCTCCGA |
| NM_006152 | AGGACTCATGGACGTCTCTAGAACATATCTTGTGGCCATTTACCAGACTC |
| NM_012203 | CATCAGCAGGGGCGACGTCGTAAACCAGGACGACCTGTACCAGGCCTTGG |
| NM_058197 | ATTGGAATCAGGTAGCGCTTCGATTCTCCGGAAAAAGGGGAGGCTTCCTG |
| NM_000277 | CGCTACGACCCATACACCCAAAGGATTGAGGTCTTGGACAATACCCAGCA |
| NM_133480 | GCTAATTGCCCAGGGCCTTTTGGAGTCTGAGGACCGCCCCGCAGAGGACT |
| NM_002447 | AACAGCCGCAGTTCTCACCCATGCCAGGGAATGTACGCCGGCCCCGGCCA |
| NM_001197 | GGAGAACATAATGAGGTTCTGGAGATCCCCGAACCCCGGGTCCTGGGTGT |
| NM_030674 | GCCTGCTCATCGAGTAATGGTGAAGGCCACTGAAACCCGCCGAGAAAAAG |
| NM_004907 | GGGACGTTGGACTGGTCCCGAGCAAGAAAGCCCGTCTGGAAGAAAAGGAA |
| NM_002910 | TTTCAAAGGCTGCTTCCACGTGCCGCGGTGCCTAGCCATGTGCGAGGAGA |
| NM_001901 | TGGGCCTGCCCTCGCGGCTTACCGACTGGAAGACACGTTTGGCCCAGACC |
| NM_005526 | TAGAGGAGGCGAGTCCCGGGCGCCCATCTTCCGTGGACACCCTCTTGTCC |
| NM_000852 | CCTCCCCTGAGTACGTGAACCTCCCCATCAATGGCAACGGGAAACAGTGA |
| NM_002511 | GGGAGGAAGTCCTATCAAGAGAGAGGAACCAGCTACCTACTCAGCTCTTC |
| NM_000365 | TCCTTGTGGGTGGTGCTTCCCTCAAGCCCGAATTCGTGGACATCATCAAT |
| NM_001891 | CCAAGAACTTCTACTTAACCCCACCCACCAGATCTACCCTGTGACTCAGC |
| NM_006851 | TACGGACCAGGAGGGAATTACCCAACTTGGCCATATAAGAGAGGAGCCAC |
| NM_005178 | GAGTGCCAAGAAACCGTGCAGCTCTTGCTAGAGCGCGGTGCCGACATCGA |
| NM_005582 | GCAAACCCGCCATCTCTAAGGGGAGTTAAGCTATCTGATGTCAAGCTTTC |
| NM_001344 | TGGGGAGTTTCATCCTAGCGGTTTGCCTGAGAATACAGATCAACCCACAG |
| NM_002086 | TTTCTTATCCGAGAGAGTGAGAGCGCTCCTGGGGACTTCTCCCTCTCTGT |
| NM_002228 | TCCTCCCGTCCGAGAGCGGACCTTATGGCTACAGTAACCCCAAGATCCTG |
| NM_002961 | GTAACGAATTCTTTGAAGGCTTCCCAGATAAGCAGCCCAGGAAGAAATGA |
| NM_002987 | GGGGCTTCTCTGCAGCACATCCACGCAGCTCGAGGGACCAATGTGGGCCG |
| NM_182908 | ACGTCAACGGGGAGAACATTGCGGTGGCAGGCTACTCCACTCGGCTCTGA |
| NM_004434 | TACCCCTGCTCGCAGTTCAGGGCTCCAAGCCACATCTACGGCGGGCACAG |
| NM_002444 | CATGCTGAGAACATGCGACTGGGCCGAGACAAATACAAGACCCTGCGCCA |
| NM_002048 | TACGATGAGGACTACGATGACGAGCAGCGCACCGGGGGCGCGGGTGGTGA |
| BM_722299 | CCTGCTCTCGCACCGTCCGAGCGGAGCTTTCGTTTTCAGTGAGCCAGGTG |
| NM_004289 | GCCAGTCAATCCCAACCACTATGCTCTCCAGTGTACCCATGATGGAAGTA |
| NM_004665 | AGGTGCTGAAAGATGGGCGTTTGGTAAACAAGAATGGATCATCTGGGCCT |
| NM_001715 | TCCTCCTGATGGAAGTTGTCACTTATGGGCGGGTGCCATACCCAGGGATG |
| NM_001783 | AACGAGTCATACCAGCAGTCCTGCGGCACCTACCTCCGCGTGCGCCAGCC |
| NM_001618 | GGTGTAGACGTTCCTCTTGGGACCGGGATTTCATCTGGTGTGATAGACAC |
| NM_002167 | CAGACAGCCGAGCTCGCTCCGGAACTTGTCATCTCCAACGACAAAAGGAG |
| NM_006115 | CCAGCTTACAACCTTAAGCTTCTACGGGAATTCCATCTCCATATCTGCCT |
| NM_015055 | AGCCCTCCACCACACAAAGAAGCCCGCCAGCGTCGGAAAGAACTCCGGAA |
| NM_000038 | CAGGGCCAGTGCAGCACTCCACAACATCATTCACTCACAGCCTGATGACA |
| NM_020992 | AAGTGTTAAAGCTCCTGTCACTAAAGTGGCTGCGTCGATTGGAAATGCTC |
| NM_002661 | AGAAGATATGTTCAGCGATCCCAACTTTCTTGCTCATGCCACTTACCCCA |
| NM_002838 | CACACCACAGCTCTGCTGCCTTACCTGCACGCACCTCCAACACCACCATC |
| NM_005215 | CCAACAGAGGATTCAGCCAATGTGTATGAACAGGATGATCTGAGTGAACA |
| NM_022743 | TGAGACTGGCTTTTGATATTATGAGAGTGACACATGGCAGAGAACACAGC |
| NM_004119 | AGATGGATTTGGGGCTACTCTCTCCGCAGGCTCAGGTCGAAGATTCGTAG |
| NM_024408 | CAGGACGGGCAGGTAGCTCAGACCATTCTCCCAGCCTATCATCCTTTCCC |
| NM_000314 | GGAAGTCTATGTGATCAAGAAATCGATAGCATTTGCAGTATAGAGCGTGC |
| NM_001795 | ACGTGGATTACGACTTCCTTAACGACTGGGGACCCAGGTTTAAGATGCTG |
| NM_001226 | GAGACAATCTTACCCGCAGGTTTTCAGATCTAGGATTTGAAGTGAAATGC |
| NM_006763 | TTGGGGAGGACGGCTCCATCTGCGTCTTGTACGAGGAGGCCCCACTGGCC |
| NM_053056 | AACAAACAGATCATCCGCAAACACGCGCAGACCTTCGTTGCCCTCTGTGC |
| NM_004126 | GGGAATTCCAGAAGACAAGAACCCCTTTAAAGAAAAAGGCAGCTGTGTTA |
| NM_006579 | GGGCCACCCTCTCTACTTCTGGTTTTACTTTGTCTTCATGAATGCCCTGT |
| NM_007360 | TGAGAGCCAGGCTTCTTGTATGTCTCAAAATGCCAGCCTTCTGAAAGTAT |
| NM_030956 | TGCTAGGTCAATGCACACAAACATGGCACAGGGTTAGGAAAACAACCCAA |
| NM_003254 | GGGTTCCAAGCCTTAGGGGATGCCGCTGACATCCGGTTCGTCTACACCCC |
| NM_001760 | ACCCGCCATCCATGATCGCCACGGGCAGCATTGGGGCTGCAGTGCAAGGC |
| NM_005225 | TTTCAGATCTCCCTTAAGAGCAAACAAGGCCCGATCGATGTTTTCCTGTG |
| NM_003810 | TAGACATGGACCATGAAGCCAGTTTTTTCGGGGCCTTTTTAGTTGGCTAA |
| NM_006325 | GCACAGTATGAGCACGACTTAGAGGTTGCTCAGACAACTGCTCTCCCGGA |
| NM_004225 | GAAGATCAAAGACAACCCACTGATCCAGCCCCCCTACGAGGTCTGCATGA |
| NM_005781 | AGCTCTTCGGGCTGGGTCTGCGGCCCAGAGGGGAGTGCCACAAAGTGCTG |
| NM_002648 | CTGTATGATATGGTGTGTGGAGATATTCCTTTCGAGCATGACGAAGAGAT |
| NM_006769 | GAATCGCCTGGTCCCGGGAGATCGGTTTCACTACATCAATGGCAGTTTAT |
| NM_004689 | GCTGGCCGGGCCTGCGAGAGCTGTTACACCACACAGTCTTACCAGTGGTA |
| NM_003403 | CCCTCATAAAGGCTGCACAAAGATGTTCAGGGATAACTCGGCCATGAGAA |
| NM_002592 | AACGGTGACACTCAGTATGTCTGCAGATGTACCCCTTGTTGTAGAGTATA |
| NM_005104 | TCCTCTGCACAGCAAGTAGCAGTGTCACGCCTTAGCGCTTCCAGCTCCAG |
| NM_004762 | ATCAGCAGGGACCCTTTCTACGAAATGCTCGCAGCACGGAAAAAGAAGGT |
| NM_004475 | CTGTGCATGCCCTCACAGGCGTGGACCTGTCTAAGATACCCCTGATCAAG |
| NM_134269 | TCTACCGCTGTCTGGTCCAGAAGGGGCTGGTAAAAACCAAAAAGTCCTAA |
| NM_017935 | CAAGACAGAGCTCGGATAGAGAGTCCAGCCTTTTCTACTCTCAGGGGCTG |
| NM_002129 | GCTATGACAGGGAGATGAAAAATTACGTTCCTCCCAAAGGTGATAAGAAG |
| MM_001469 | AAAGACTGGGCTCCTTGGTGGATGAGTTTAAGGAGCTTGTTTACCCACCA |
| MM_006819 | GCTACCAGCGCTGTATGATGGCGCAGTACAACCGGCACGACAGCCCCGAA |
| NM_004941 | ATGAGGAACCCAATGCCTGGAGAATATCTCGAGCTTTCCGACGGCGCTGA |
| NM_003352 | CTCTTTGAGGGTCAGAGAATTGCTGATAATCATACTCCAAAAGAACTGGG |
| NM_000626 | GCTTCACGGTGAAAATGCACTGCTACATGAACAGCGCCTCCGGCAATGTG |
| NM_001237 | CTACCTCAAAGCACCACAGCATGCACAACAGTCAATAAGAGAAAAGTACA |
| BC011857 | TCCTGAGCCTCTTCTACAGTACCACCGTCACCTTGTTCAAGGTGAAATGA |
| NM_002466 | TACTCCATGGACAACACTCCCCACACGCCAACCCCGTTCAAGAACGCCCT |
| NM_000424 | CCAGTCAAGTGTGTCCTTCCGGAGCGGGGGCAGTCGTAGCTTCAGCACCG |
| AB037771 | GCCAACCAGAACTAGACTCTATTTCTACCTGTCCAAATGAGACAGTTTCA |
| NM_025113 | AGCCCATTTTCAATTTGCTGAGCATCGGCCAAAGCCTGTATGCGAAAGCC |
| NM_032873 | ACCAATCCTTCCTCTTACCCATGGACCAACTGGGGGCTTCAACTGGAGAG |
| NM_023037 | TCCTCACTACTTTTCTTCCAGACTCCAGTGTTTCTGGCACTAGTCTCTGA |
| NM_033306 | TTGACCATCTGCCTCCGAGGAATGGAGCTGACTTTGACATCACAGGGATG |
| NM_003217 | CGTCCTTGTTGATACTCAACTCATTATTGAAAAGGCCGAACATGGAGATC |
| AF509494 | AAGAAGAAAGGCGACAGCAACTTCGTAGGAAGGTAGTTGAAATTCTTCCA |
| NM_001781 | CACATTCTCAATGCCATCAGACAGCCATGTTTCTTCATGCTCTGAGGACT |
| NM_002984 | CAGCCAGCTGTGGTATTCCAAACCAAAAGAAGCAAGCAAGTCTGTGCTGA |
| BC025340 | CGCAGCATGGAGTCTGCTCTCAGCTGTTGGGGAATTACCGATGCCTTTGA |
| NM_030926 | CCCCTCGCAACTTCTGGGAGCTCCTCATGAACGTGAAGAGGGGGACCTAC |
| AF331856 | GGTATTTGGTTTCCCAGTCCACTATACTGACGTCTCCAACATGAGCCGCT |
| NM_000405 | GAAAAAGCCATCCCAGCTCAGTAGCTTTTCCTGGGATAACTGTGATGAAG |
| NM_000483 | CCGCTGTAGATGAGAAACTCAGGGACTTGTACAGCAAAAGCACAGCAGCC |
| NM_001888 | CAGCCCACTGTGAGAAGACCACGGTGTTCAAGTCTTTGGGAATGGCAGTG |
| NM_014670 | GTCTGAGCTGACTCTGTTACTGAAGATTCAGGAGTATTGCTATGACAACA |
| NM_003804 | AGCTATCTTTGATAATACCACTAGTCTGACGGATAAACACCTGGACCCAA |
| AB018263 | TCAGGACCACCCTGCTGCTTAACTCCACGCTCACTGCCTCGGAGGTCTGA |
| NM_006475 | GCAGTCTTCAGCCTATTATCAAAACTGAAGGACCCACACTAACAAAAGTC |
| NM_001786 | AATCCTACAGGGGATTGTGTTTTGTCACTCTAGAAGAGTTCTTCACAGAG |
| NM_031942 | CGGCAGCGAGATGGACGGTGTGCGACTGGGGTCCTTGTGTATTTAGCCAA |
| NM_001640 | CGCATGGAGAACATTCGATTCTGCCGCCAATACCTGGTGTTCCATGACGG |
| NM_002483 | GTGCTCCTGTCCTCTCAGCTGTGGCCACCGTCGGCATCACGATTGGAGTG |
| NM_003915 | CACACCCATCCAGGTGCAATGCTCCGATTATGACAGTGACGGGTCACATG |
| NM_147180 | CCACAATGGGAAACGAGGCCAGTTACCCGGCGGAGATGTGCTCCCACTTT |
| NM_000485 | TATTCAGGGACATCTCGCCCGTCCTGAAGGACCCCGCCTCCTTCCGCGCC |
| NM_004924 | AACCCCTACACCACCGTCACCCCGCAAATCATCAACTCCAAGTGGGAGAA |
| NM_003480 | CTGCCCCCTAGGAGACTCCGTCGCTCCAATTACTTCCGACTTCCTCCCTG |
| NM_002023 | CAACAATGTCTACACCGTCCCCGATAGCTACTTCCGGGGGGCGCCCAAGC |
| NM_006216 | GCCGCTGAAAGTTCTTGGCATTACTGACATGTTTGATTCATCAAAGGCAA |
| NM_003651 | GCAGGTGAAGCACCAACTGAGAACCCTGCTCCACCCACCCAGCAGAGCAG |
| NM_006565 | GAGAGGACGACCCCCTGGCAGAACCAACCAGCCCAAACAGAACCAGCCAA |
| NM_030666 | CTTTTCTTTATTCGGCATAATTCCTCAGGTAGCATCCTATTCTTGGGGAG |
| NM_005537 | GGGGTGGAGGGTGGACGAGTTGATTTGAACGTCTTCGGGTCGCTCGGCCT |
| NM_004235 | CGTGGCCCCGGAAAAGGACCGCCACCCACACTTGTGATTACGCGGGCTGC |
| NM_016269 | CTATCAACCAGATTCTTGGCAGAAGGTGGCATGCCCTCTCCCGTGAAGAG |
| NM_006164 | TCAGCACCTTATATCTCGAAGTTTTCAGCATGCTACGTGATGAAGATGGA |
| NM_004630 | TCCTCACCCAACTCCCTTTGCCTCTCCCCAAACCGGGCCGCCAGGATCCC |
| NM_003222 | CGAACATACAGAACTGCTTGTCTCATTTCAGCCTGATTACCCACGGGTTT |
| NM_000700 | GCCATAAGGCATTGATCAGGATTATGGTTTCCCGTTCTGAAATTGACATG |
| NM_019846 | GGGCACATCAGGGGAAACACGAAACATACGGCCATAAAACTCCTTATTAG |
| NM_006889 | CCTGAAAGATCTGATGAAGCCCAGCGTGTTTTTAAAAGTTCGAAGACATC |
| NM_032738 | CAGAAACAGCATCTGTTGTGGCTATCACAGTCCAAGAACTGTTTCCAGCG |
| NM_000417 | TTATTATCAGTGCGTCCAGGGATACAGGGCTCTACACAGAGGTCCTGCTG |
| BC019046 | TCACCATCTTCATCACACTCTTCCTGTTAAGCGTGTGCTACAGTGCCACC |
| NM_002189 | GAGCGCTGTGTCTCTCCTGGCATGCTACCTCAAGTCAAGGCAAACTCCCC |
| NM_004827 | AGGGCATCGATCTCTCACCCTGGGGCTTGTGGAAGAATCACGTGGCCTTG |
| NM_001647 | ATCAAATCGAAGGTGAAGCCACCCCAGTTAACCTCACAGAGCCTGCCAAG |
| NM_014257 | AGACCAGTCCAAGCAGCAGCAAATCTATCAAGAACTGACCGATTTGAAGA |
| NM_001830 | TTGCCCAGAGGAGGGAACTGATTCTCGCAATAAATAACGCCAGACAGAGG |
| NM_020944 | CAAACAGGGCACAGGACTAAGGACAGGGCCTATGTTTGGACCAAAGGAAG |
| NM_021643 | CATCCTTGGTTTTCTACAGATTTTAGCGTCTCGAATTCAGCATATGGTGC |
| NM_017784 | AGGAACGGAAGCGCGAGAACCTCCGCACACCATGGAAGCCCAAATATTTT |
| NM_022366 | GCGTTCCAAAGATTGTGCTTATAAATGGCTGTATGATGAAACCCTGGAAG |
| NM_002933 | TGTGGCCTGTGAAGGGAGCCCATATGTGCCAGTCCACTTTGATGCTTCTG |
| NM_022829 | CCTTCCCGGACTGGGCTGATATGTACTCGGTCAATGTCACAGCATTGCCA |
| NM_003339 | AGATGATCCTTTAGTGCCTGAGATTGCTCGGATCTACAAAACAGATAGAG |
| NM_005277 | AATCTCTGCTTGGACCTTCGTCAGTTTGGAATTGTGACAATTGGAGAGGA |
| NM_006820 | CAAAGCCGGGTCATGAATGTCCATAAAATGCTAGGCATTCCTATTTCCAA |
| NM_024728 | CCCATGGCCCCTTCCTTCATCTTCAAGGATCAGCAAGGGTATTACACAAT |
| NM_025263 | GCTTTCACAAGGAACAGAGAAACCCTCGAAGGCTCAAAAGCTGGTCTCTT |
| BC046632 | AGCAGTGCCGGTGCATGTCCGTGAACCTGAGCGACTCGGACAAGCAGTGA |
| NM_000877 | TGGTCAGGGGACTTTACACAGGGACCACAGTCTGCAAAGACAAGGTTCTG |
| NM_014365 | TTGACAGCCTCTTGGCCCGACTGGGCTCTGCCTCGTCTCTCCTCCGCCTG |
| AK092000 | ATCGAGCATGCTATTCCAGTGTACTGAACATACTGTATACCTCGTGTTAG |
| NM_022436 | GCTCAAATGTTTCTGTGACAACTAATCCAATGTGTGCCTTCACTCAAGGA |
| NM_001165 | CAACATTTGACTTGTGTAATTCCAATCCTGGATAGTCTACTAACTGCCGG |
| BX647445 | ACTGTTGCCCTGGCTGTATTCATAAGATTCCAGCTCCTTCAGGTGTTTGA |
| BC022095 | CATGCGCACGGGGTTCCTAGCTCCCACGTCTATGTTCACATTTGTGGTCC |
| NM_031305 | CAGACTGTCCACCTATGATAATGTCCATCAACAGTTCTCCATGATGAACC |
| NM_006191 | CCAATGGCCCCATGCGGATAACCAGTGGTCCCTTCGAGCCTGACCTCTAC |
| NM_014792 | CCGGTGAGCTTGTGGTGTGGGTTTTCAGGCTGTATCCTTCTACCTCCTGA |
| NM_014686 | TGTGAACCAGGATAACAAAACCAAAACGTGGCCACCCAAAGCACCCTGGC |
| AB029034 | CAGGCAAACCGCAGCACCACACCTATGGCCCCCGGTGTCTTCTTGACCCA |
| BQ722784 | ATCGGTCTTTCTCTTTCAATCCTCACCCGGGATATTTGACCCCTTCCATG |
| NM_198243 | CTGTGTGCACGGATGTTATATAATTACGGAGCAGACACGAACACACGGAA |
| NM_016570 | GCACATGCCATTCTGGCAGTTTTTTGTAAGACTCTGTGGTATTGTTGGAG |
| BC001077 | CCACTGCCCTCTAAGACCTTGGAAGGGGAAACACCAGAAGGTGTGGGTGC |
| NM_138379 | ACATTGGAGATAGTAAAAATGTCCTCAATGACGTGCAGCATGGAAGGGAA |
| NM_024319 | ATGTTCGGCTCCAGTCGTGGAGGCGTGCGCGGCGGGCAGGACCAGTTCAA |
| AB032991 | GGAAGGTATGGTGCTATCTGCGGATTTGGCCTTTCCTTGATCAAATGGAT |
| NM_015670 | CCTATTTTGACTCGCAGCGTACCCTAAACCGCCGCTGCCCTAAGCATATT |
| NM_015436 | CCTTGCTGTGACAAGCTTTATACTTGCCGCTTGTGTCATGATAACAATGA |
| NM_032263 | AAGATTCGGCCACTTACAACAGTCTCCTGCAAGCTTTGAGCAAAGAGAGG |
| NM_021960 | AAGGACAAAACGGGACTGGCTAGTTAAACAAAGAGGCTGGGATGGGTTTG |
| NM_000295 | TGACCACCGTGAAGGTCCCTATGATGAAGCGTTTAGGCATGTTTAACATC |
| NM_002390 | ACCCACGGGGGAGACGGAGAGATATAAAGGTCCCAGCGGCACCAACATCA |
| NM_001706 | CTTCCGCTACAAGGGCAACCTCGCCAGCCACAAGACCGTCCATACCGGTG |
| NM_005214 | TGCAGCAGTTAGTTCGGGGTTGTTTTTTTATAGCTTTCTCCTCACAGCTG |
| NM_006850 | TCCGGAGAGCATTCAAACAGTTGGACGTAGAAGCAGCTCTGACCAAAGCC |
| NM_000418 | GGACAGGGAGCCACCTCGCAGTCCGCAGAGCTCACATCTCCCAAGCAGCT |
| NM_002835 | CCAACAGAAGCCACAGATATTGGTTTTGGTAATCGATGTGGAAAACCCAA |
| NM_002927 | CTGGATGGCATGTGAAACCTATAAGAAAATTGCCTCACGGTGGAGCAGAA |
| NM_003005 | CTAGGAACATATGGAGTTTTTACAAACGCTGCATTTGACCCGAGTCCTTA |
| NM_005449 | TCCAGATGCCTGCATATGCCAGTTCTTCCAAATTCGTAACCAGAGTTACC |
| NM_000075 | CAGAGGATGACTGGCCTCGAGATGTATCCCTGCCCCGTGGAGCCTTTCCC |
| NM_004064 | AATAAGGAAGCGACCTGCAACCGACGATTCTTCTACTCAAAACAAAAGAG |
| NM_004454 | GGAGGACACCCTGCCGCTGACCCACTTTGAAGACAGCCCCGCTTACCTCC |
| NM_001775 | GCCAGGATCCCACCATAAAAGAGCTGGAATCGATTATAAGCAAAAGGAAT |
| NM_176783 | ACTCAAATCTCTAAGTATTTCTCTGAGCGTGGTGATGCAGTGACTAAAGC |
| NM_016187 | TTGTACTTCCCCCACCTTAATGACATCTCAGGTTGCTTCAGAGCCTGGAG |
| NM_006495 | GGAGTTCTCTATTCCTCCCAACTCTGATCAAGATCTTAATGAATCCCTGC |
| NM_042066 | CTCCATCAAGAGAGCTACTGAAGCATCCAGTCTTTCGTACTACATGGTAG |
| NM_005248 | CAGCATCCCTGTACGAGGCCATGGAACAGACCTGGCGTCTGGACCCGGAG |
| NM_032663 | AAAGAAGCGTAGAAAAGGGCTTGTGCCTGGCCTTGTTAATTTAGGGAACA |
| NM_000633 | CCCTGGTGGGAGCTTGCATCACCCTGGGTGCCTATCTGGGCCACAAGTGA |
| NM_005100 | AATGGCCAGAAAGGAGCCCTGAACGGTCAAGGAGCCCTAAACAGCCAGGA |
| NM_014207 | CATCGCAACCACACGGCAACCGTCCGATCCCATGCTGAGAACCCCACAGC |
| NM_001831 | TGACTCTGATCCCATCACTGTGACGGTCCCTGTAGAAGTCTCCAGGAAGA |
| NM_006187 | ACAGAGCTACAACGGGACTTCATCATCTCTCGCCCTACCAAGCTGAAGAG |
| NM_058176 | GCCTGTGGCCAAAGAGTTTGATCCAGACATGGTCTTAGTATCTGCTGGAT |
| NM_002738 | GATTTTTCACCCGCCATCCACCAGTCCTAACACCTCCTGACCAGGAAGTC |
| NM_001774 | TGTGCAGAAACCTGGACCACGTCTACAACCGGCTCGCTCGATACCGTTAG |
| NM_152866 | AAGAAACAGAGACGAACTTTCCAGAACCTCCCCAAGATCAGGAATCCTCA |
| NM_006184 | GCCGAGCAGGATCCCAATGTACAGGTGGATCATCTGAATCTCCTGAAACA |
| NM_003955 | CCTATGAGAAAGTCACCCAGCTGCCGGGGCCCATTCGGGAGTTCCTGGAC |
| NM_002162 | AGAGCACCTATCTGCCCCTCACGTCTATGCAGCCGACAGAAGCAATGGGG |
| NM_000598 | AGTTCCTCAATGTGCTGAGTCCCAGGGGTGTACACATTCCCAACTGTGAC |
| NM_006472 | CCTGAGTTCAAGTTCATGCCACCACCGACTTATACTGAGGTGGATCCCTG |
| NM_005998 | CTGTGAGACCTGGGGTGTAAATGGTGAGACGGGTACTTTGGTGGACATGA |
| NM_000884 | TGGAAGGTGGCGTCCATAGCCTCCATTCGTATGAGAAGCGGCTTTTCTGA |
| NM_006636 | CTGCAAAAAAGGTGCTGAGGCTTGAAGAGCGAGAAGTGCTGAAGTCTAAA |
| NM_000269 | AAGGACCGTCCATTCTTTGCCGGCCTGGTGAAATACATGCACTCAGGGCC |
| NM_004619 | GCCAAGAACGCCTACATTAAAGATGACACTCTGTTCTTGAAAGTGGCCGT |
| NM_004972 | GGGATCTAGCTCTTCGAGTGGATCAAATAAGGGATAACATGGCTGGATGA |
| NM_019841 | CTGGGGCACTTGAATCTTGGACTGAACCTTAGTGAGGGGGATGGAGAGGA |
| NM_156039 | CGACTGTGTCTTTGGGCCACTGCTCAACTTCCCCCTCCTGCAGGGGATCC |
| NM_002467 | AGGAGGAACGAGCTAAAACGGAGCTTTTTTGCCCTGCGTGACCAGATCCC |
| NM_002460 | GCAATCCAGAAGATTACCACAGATCTATCCGCCATTCCTCTATTCAAGAA |
| NM_001242 | GGGCCCTGTTCCTCCATCAACGAAGGAAATATAGATCAAACAAAGGAGAA |
| NM_001877 | TAGAAGCACGAGAAGTATATTCTGTTGATCCATACAACCCAGCCAGCTGA |
| NM_004513 | TGGGTGGCACTGCCATGCAGGGCCTCACACGGTTTGAAGCCTGGAACATC |
| NM_007289 | CAGAAATGCTTTCCGCAAGGCCCTTTATGGTACAACCTCAGAAACAGCAA |
| NM_018209 | GCCCCTCGGAGGGCCACAGTTATCAGAACAGCGGTCTGGACCACTTCCAA |
| NM_002166 | TTGGACCTGCAGATCGCCCTGGACTCGCATCCCACTATTGTCAGCCTGCA |
| NM_001882 | GAAAACCCAAATGGAAACAGTATCGGGGAATTCTGTTTGTCTGGTCTTTG |
| NM_000698 | ACGGTCACCGTGGCCACTGGCAGCCAGTGGTTCGCCGGCACTGACGACTA |
| NM_001923 | GCAAACCTACAGTATGACGATGGCAGCGGTATGAAGCGAGAGGCCACTGC |
| NM_031966 | ATGCCACATCGAAGCATGCTAAGATCAGCACTCTACCACAGCTGAATTCT |
| NM_001987 | AAAATATTCCGGATAGTGGATCCCAACGGACTGGCTCGACTGTGGGGAAA |
| NM_002574 | CTGTTGGCCGCTCTGTGGATGAGACTTTGAGACTAGTTCAGGCCTTCCAG |
| NM_000034 | TCCCTTCCCCCAAGTTATCAAATCCAAGGGCGGTGTTGTGGGCATCAAGG |
| NM_003656 | AGATACAGCTCTAGATAAGAATATCCACCAGTCGGTGAGTGAGCAGATCA |
| NM_002168 | CCGCGGCGCCACTATGCCGACAAAAGGATCAAGGTGGCGAAGCCCGTGGT |
| NM_005566 | CGGAATAAAGGATGATGTCTTCCTTAGTGTTCCTTGCATTTTGGGACAGA |
| NM_003070 | GCGGAGGGAAGATGCCCGGAACCCGAAACGGAAGCCCCGTTTAATGGAGG |
| NM_007146 | AATGAGACCTGTAGAGAGCATGCCTTTCTTGCCCCAAGCTTTGCCTACAT |

TABLE 26

Polynucleotide probe sequences for
preparation of a small 30mer
nucleic acid array

| GenBank | |
|---|---|
| Accession No. of | Oligonucleotide |
| Target gene | sequence (5′ to 3′) |
| NM_001728 | AAGAACGTCCGCCAGAGGAACTCTTCCTGA |
| NM_018462 | GACACTCACTAGAACAGTGCCGTGCTGCTG |
| NM_002266 | TACACTTTCCAAGTTCAGGATGGGGCTCCT |
| NM_004310 | AGGAGACGAAACAGAAGGAGGCTCTTCTCC |
| NM_006152 | CTCCGACACAATGGGCCACCACCAGTGTGA |
| NM_012203 | AGGGGAGCCGATGCCTAGTGAACTCAAGCT |
| NM_058197 | GGAGTTTTCAGAAGGGGTTTGTAATCACAG |
| NM_000277 | CCAGCAGCTTAAGATTTTGGCTGATTCCAT |
| NM_133480 | CACTTAGTGCCCACAACCGCACCAAGAAGC |
| NM_002447 | TCTCACCCATGCCAGGGAATGTACGCCGGC |
| NM_001197 | TGAGGTTCTGGAGATCCCCGAACCCCGGGT |
| NM_030674 | CACACATCAGCAATCTCTCACCACTTCTTT |
| NM_004907 | AGCGTCATCCGAAGTCGCCGATCGCCTGCA |
| NM_002910 | AAAGGCTGCTTCCACGTGCCGCGGTGCCTA |
| NM_001901 | AATCGCTGTACTACAGGAAGATGTACGGAG |
| NM_005526 | CCCAAAGCCAAGGACCCCACTGTCTCCTAG |
| NM_000852 | CTCCCCATCAATGGCAACGGGAAACAGTGA |
| NM_002511 | CACAGCATGAAGCAGGAAATGGCAATGTGA |
| NM_000365 | CTCAAGCCCGAATTCGTGGACATCATCAAT |
| NM_001891 | CCACCCACCAGATCTACCCTGTGACTCAGC |
| NM_006851 | CCACGTAACAGATACACTTCTCTCTTTCTC |
| NM_005178 | TCAGCTCCAATGGTCTTCTCTCCGCATCAC |
| NM_005582 | CTGTGGGATTACAGCCATAGGCATTTTCTT |
| NM_001344 | GTTTGCCTGAGAATACAGATCAACCCACAG |
| NM_002086 | CCGAGAGAGTGAGAGCGCTCCTGGGGACTT |
| NM_002228 | TGATAATCCAGTCCAGCAACGGGCACATCA |
| NM_002961 | CTTTGAAGGCTTCCCAGATAAGCAGCCCAG |
| NM_002987 | TGCTCCAGGGATGCCATCGTTTTTGTAACT |
| NM_182908 | TTGCGGTGGCAGGCTACTCCACTCGGCTCT |
| NM_004434 | ACAAGCATCATGCAGTGGCGCGTCATTTAG |
| NM_013451 | CAGCCCAACATTTCCAGATACTACCTGCGT |
| NM_002444 | ATGACATGATCCATGCTGAGAACATGCGAC |
| NM_002048 | TGAGGACTACGATGACGAGCAGCGCACCGG |
| BM722299 | GTGGTTCACAACGACAGGCTACTGAGCACC |
| AB033114 | TAGGAGAAGCACTGTTGTTTTCCACACAGT |
| NM_004665 | GACCAGCAATTCAGCAATAACTTACCTGCT |
| NM_001715 | GATGAGCAACCCCGAGGTCATCCGCAACCT |
| NM_001783 | AGGAGGGCAACGAGTCATACCAGCAGTCCT |
| NM_001618 | GACCGGGATTTCATCTGGTGTGATAGACAC |
| NM_002167 | CGGAACTTGTCATCTCCAACGACAAAAGGA |
| NM_006115 | GTGGGGACAGAACCTTCTATGACCCGGAGC |
| NM_015055 | ACCCTCACCTGATCACTAACTGGGGACCTG |
| NM_000038 | AGAGAGGCAGGCGTGAAATCCGAGTCCTTC |
| NM_020992 | TATGAAGTGGTCACTGTGTTCCCCAAGTGA |
| NM_005933 | ACTGGGGCAAGAACGGAGTTGCTTCTATAA |
| NM_002661 | CAGTGTTAATGAGAACCACTCCAGCTGTAC |
| NM_002838 | CACCATCACAGCGAACACCTCAGATGCCTA |
| NM_005215 | CCAATGTGTATGAACAGGATGATCTGAGTG |
| NM_022743 | GAATGCGACGCCAACATCAGAGCATCCTAA |
| NM_004119 | TCTCCGCAGGCTCAGGTCGAAGATTCGTAG |
| NM_024408 | CACATGTCTGAGCCACCACACAACAACATG |
| NM_000314 | CCACTGACTCTGATCCAGAGAATGAACCTT |
| NM_001795 | GACTGGGGACCCAGGTTTAAGATGCTGGCT |
| NM_001226 | CGCAGGTTTTCAGATCTAGGATTTGAAGTG |
| NM_006763 | AAGAACTACGTGATGGCAGTCTCCAGCTAG |
| NM_053056 | TGATCAAGTGTGACCCAGACTGCCTCCGGG |
| NM_004126 | AGCTTCGCAAAGAAGTGAAGTTGCAGAGAC |
| NM_006579 | ATGCTGTGAAGCACCTCACTCATGCCCAGA |
| NM_007360 | GTGCACTCTATGCCTCGAGCTTTAAAGGCT |
| NM_030956 | GAGGTTCTACAATCTCTCTGATGAGAACAG |
| NM_003254 | AACTGCAGAGTGGCACTCATTGCTTGTGGA |
| NM_001760 | CCTACAGATGTCACAGCCATACACCTGTAG |
| NM_005225 | CTCATCCCTCACCACAGATCCCAGCCAGTC |
| NM_003810 | CAGTTTTTTCGGGGCCTTTTTAGTTGGCTA |
| NM_006325 | AGAGGTTGCTCAGACAACTGCTCTCCCGGA |
| NM_004225 | ACCCACTGATCCAGCCCCCCTACGAGGTCT |
| NM_005781 | AAGTGCTGGAGATGTTCGACTGGAACCTGG |
| NM_005243 | AGTACTCAGCCGGCAGCATAATGAAAAGTG |
| NM_002648 | GATAGGCCAACCTTCGAAGAAATCCAGAAC |
| NM_006769 | TGAATTCACTTCAGAGCAATCCACTACTGC |
| NM_004689 | AACCGCAGTAACATGAGTCCCCACGGCCTC |
| NM_003403 | AACATCTGCACACCCACGGTCCCAGAGTCC |
| NM_002592 | TACTTGGCTCCCAAGATCGAGGATGAAGAA |
| NM_005104 | TCAGACACCAGTGATTCAGACTCAGGCTAA |
| NM_004762 | AAGAAGGTCTCCTCCACGAAGCGACACTGA |
| NM_004475 | ATCAAGAAGGCCACTGGTGTGCAGGTGTGA |
| NM_013995 | GTGCTGGTCTTTCAGGCTTGATTATCGTTA |
| NM_134269 | GAAGGGGCTGGTAAAAACCAAAAAGTCCTA |
| NM_017935 | CGACCCCAAGTTGAAAAGGAATTTGGTTTC |
| NM_002129 | GCTGCATATCGTGCCAAGGGCAAAAGTGAA |
| NM_181430 | GCCAGCTCAGACTGGAGTTGCCTCAGTTGA |
| NM_031844 | AGGTAAGAAGAAGGCGGAAGGCGGCGGAGG |
| NM_006819 | ATGGATGTGGGTCTGATTGCAATTCGGTGA |
| NM_004941 | AGAATATCTCGAGCTTTCCGACGGCGCTGA |
| NM_003352 | GAACAAACGGGGGGTCATTCAACAGTTTAG |
| NM_000626 | ACAGAGCTGCGAGTCATGGGATTCAGCACC |
| NM_001237 | AAGTATCATGGTGTTTCTCTCCTCAACCCA |
| BC011857 | TCCCGACGGTGGGTTTGTTCTACTGGGGCC |
| NM_002466 | CACACATCTCGGACCCTCATCTTGTCCTGA |
| NM_000424 | TGGCTTCAGTGCAAGCAGTGGCCGAGGGCT |
| AB037771 | CCAAGCTCTCTTTTCCAGAGAGCAGTGGCT |
| NM_025113 | GACTGTTCAACATGCCTTATGATAACACCG |
| NM_032873 | GGACCAACTGGGGGCTTCAACTGGAGAGAG |
| NM_023037 | GACTCCAGTGTTTCTGGCACTAGTCTCTGA |
| NM_033306 | GGCTCTATCTTCATCACACAACTCATCACA |
| NM_003217 | GGCTTCGTCCTTGTTGATACTCAACTCATT |
| AF509494 | CTTGAGTAAGACAAGTCTGGTGAACGAATG |
| NM_001781 | ACAACTGGTTCAACGTTACAGGGTCTGACA |
| NM_002984 | GAGTACGTGTATGACCTGGAACTGAACTGA |
| BC025340 | CAGCTGTTGGGGAATTACCGATGCCTTTGA |
| NM_030926 | GCTCCTCATGAACGTGAAGAGGGGGACCTA |
| AF331856 | ACTATACTGACGTCTCCAACATGAGCCGCT |
| NM_000405 | AAGATCGCTGCCTCTCTAAAGGGCATATAG |
| NM_000483 | AGGGACTTGTACAGCAAAAGCACAGCAGCC |
| NM_001888 | CAAACTCATCTATGATTCCTGGTCATCTGG |
| NM_014670 | TTGCTGCCTTTACTACTCAAGGTCAGTCTG |
| NM_003804 | AGCAGCTTGATTTACGTCAGCCAGAACTAA |
| AB018263 | AACTCCACGCTCACTGCCTCGGAGGTCTGA |
| NM_006475 | CAATAACTGAAGTGATCCATGGAGAGCCAA |
| NM_001786 | GGGGATTGTGTTTTGTCACTCTAGAAGAGT |
| NM_031942 | TGAGATACAAGATGGAATGAGGCTGCAGTC |
| NM_001640 | AGGGCATGGAGTATTACCGTGCCCTCAAGA |
| NM_002483 | GTGGCCACCGTCGGCATCACGATTGGAGTG |
| NM_003915 | GCTCCGATTATGACAGTGACGGGTCACATG |
| NM_018842 | CCAGTGAGAAGCTTCAGTGGAGAGGCCTAA |
| NM_147180 | TATCCTTTGAGGAATTCAGTGCTGTGGTCA |
| NM_000485 | TGGCACCTGTACCCTTCTTCTCTCTCCTGC |
| NM_004924 | ACGGCCTTGTATGGCGAGAGCGACCTGTGA |
| NM_002276 | ATCACTACAACAATTTGTCTGCCTCCAAGG |
| NM_003480 | CCTCCCTGTGAAAATGTGGATTTGCAGAGA |
| NM_002023 | CTCCAAGGCAATAGGATCAATGAGTTCTCC |
| NM_006216 | GTGTTATTCATGGGGCAGATAAACAAACCC |
| NM_001675 | AAGGCAAGGGGGAAGAAAAGGGTCCCCTAG |
| NM_021813 | TAAGTGTACAACTGACGAACAGCCCAGGAA |
| NM_006565 | GAAGACCAGAATACAGGTGCAATTGAGAAC |
| NM_030666 | GGTAGCATCCTATTCTTGGGGAGATTTTCT |
| NM_005537 | CTTTCGGGCGCGGATTTATAGCAGTAGCAG |
| NM_004235 | CCTCGCCTTACACATGAAGAGGCATTTTTA |
| NM_016269 | TGCATCAGGTACAGGTCCAAGAATGACAGC |
| NM_006164 | TTCTCCTAGTGAATACTCCCTGCAGCAAAC |
| NM_004630 | CTCTCCTCACCCAACTCCCTTTGCCTCTCC |
| NM_003222 | CCAGCTGATTCTAACAAAACCCTGGAGAAA |
| NM_000700 | GCCAAGCCATCCTGGATGAAACCAAAGGAG |
| NM_019846 | GAAACACGAAACATACGGCCATAAAACTCC |
| NM_006889 | GCCCAGCGTGTTTTTAAAAGTTCGAAGACA |
| NM_032738 | AACAGCATCTGTTGTGGCTATCACAGTCCA |
| NM_000417 | GCCCCAGCTCATATGCACAGGTGAAATGGA |
| BC019046 | TACAGGAACATGATCGGACAGGGGGCCTAG |
| NM_002189 | GACTTGGAAAACTGCTCTCACCACCTATGA |
| NM_004827 | TGGGGCTTGTGGAAGAATCACGTGGCCTTG |
| NM_001647 | TTGGCAAGAAACCCTAATCTCCCTCCAGAA |
| NM_001830 | CAGGACCCCGAATCCATCATGTTTAATTAG |
| NM_020944 | GGACAGGGCCTATGTTTGGACCAAAGGAAG |
| NM_021643 | GGAAGAGAACTTGGACCCTTTCTTTAACTG |
| NM_017784 | TGGAAGCCCAAATATTTTATCCAGGAGGGC |
| NM_022366 | CTGTATGATGAAACCCTGGAAGATAGGTAG |
| NM_002933 | CATATGTGCCAGTCCACTTTGATGCTTCTG |
| NM_022829 | TTGGCCAATGACACATTTCGGACCCTCTGA |
| NM_003339 | CCTGAGATTGCTCGGATCTACAAAACAGAT |
| NM_005277 | TCCAAAGAGCGGCTCAATGCATACACATAA |
| NM_006820 | TTCTCATCCTCTCTGCACTGAGGCAGATGC |
| NM_024728 | CATCTTCAAGGATCAGCAAGGGTATTACAC |
| NM_025263 | CATCCAGGCGTCAATGGACCTCCTCTGTGA |
| BC046632 | GTGAACCTGAGCGACTCGGACAAGCAGTGA |
| NM_024713 | CCATCTTACTTTGGTTCCAGGATATACTGG |
| NM_000877 | TTCATCTAAACACCAGTTACTGTCACCAGC |
| NM_014365 | CAGGACAGCCAGGAAGTCACCTGTACCTGA |
| AK092000 | CAGTGTACTGAACATACTGTATACCTCGTG |
| NM_022436 | CCAGCTCTTGTCATCCTAGGAATAGTTGTT |
| NM_001165 | GTGTCCTATTTGTAGGAGTACAATCAAGGG |
| BX647445 | CATAAGATTCCAGCTCCTTCAGGTGTTTGA |
| NM_001743 | ATCGATGGAGATGGCCAGGTCAATTATGAA |
| BC022095 | GTCTTGCGAGGACAGCCTCATAGAGAATGA |
| NM_031305 | CGAGAGAGGAAACACAATATGGATTCAGTG |
| NM_006191 | GAGAATCCCACCAGTGGGGAAACATTAGAA |
| NM_014792 | GTTTTCAGGCTGTATCCTTCTACCTCCTGA |
| NM_014686 | AGTGTGGGCCGCAACCAACGACTGCAGTGC |
| AB029034 | AGAGACTCGGCCGTATCCTGAAAATCCACA |
| BQ722784 | ACCCCAGATGGTGAAACGGCCCAAGCCAAA |
| NM_198243 | TAATTACGGAGCAGACACGAACACACGGAA |
| NM_016570 | CGTTTCAGACTTGGATCCTATAAACCTGTC |
| BC001077 | CGCGGTAGACGAGCGGATGCTGCGCTTTCT |
| NM_138379 | GAAGACGAAGACGGCCTTTTTACCCTCTAA |
| NM_024319 | TCTCGCAGGTGGCATGACAGAGGCTCTGAG |
| NM_005340 | CTTGGAGGTCGGCAAATGCATTGGCCTCCT |
| AB032991 | CAGAAACATGTCTGAAAGTATGGCAGCTGC |
| NM_015670 | GAGCTGTGTCACTGCAAACTCACTGTGTGA |
| NM_015436 | CAAGCTTTATACTTGCCGCTTGTGTCATGA |
| NM_032263 | GGCGAGGCACTATGATACGGAGAGAAATTG |
| NM_021960 | TAGTTAAACAAAGAGGCTGGGATGGGTTTG |
| NM_000295 | GGTCTGCCAGCTTACATTTACCCAAACTGT |
| NM_002390 | ATTCGCCGAGGAAGGTCCGGAGGGGCCTAA |
| NM_001706 | ACCAAGGTGCAATACCGCGTGTCAGCCACT |
| NM_005214 | CCTCACAGCTGTTTCTTTGAGCAAAATGCT |
| NM_006850 | GGGAAGTGGACATTCTTCTGACCTGGATGC |
| NM_000418 | CTCCGTGGGACCCACATACATGAGGGTCTC |
| XM_034274 | CCAGTAGTACTTCAAGAGCTCTCATACTGT |
| NM_002835 | CCAAGAGATCCACCTTCAGAATGGACATGA |
| NM_002927 | GGATTCCTACCCCAGATTTCTAAAGTCAGA |
| NM_003005 | ACAAACGCTGCATTTGACCCGAGTCCTTAA |
| NM_000075 | CTACATAAGGATGAAGGTAATCCGGAGTGA |
| NM_004064 | GACGGTTCCCCAAATGCCGGTTCTGTGGAG |
| NM_004454 | CCCACTTTGAAGACAGCCCCGCTTACCTCC |
| NM_001775 | GAGGATTCATCTTGCACATCTGAGATCTGA |
| NM_176783 | CAGGGGAGAAACAAAGGGAATGATCTATTG |
| NM_001826 | ACGAGGAGTTTGAGTATCGACATGTCATGC |
| NM_016187 | CTTCAAGTCTCCATGGTACCAGAAAACAAC |
| NM_006495 | CTCTATTCCTCCCAACTCTGATCAAGATCT |
| XM_042066 | AAGCATCCAGTCTTTCGTACTACATGGTAG |
| NM_005248 | GACTACTTCACCTCCGCTGAACCACAGTAC |
| NM_032663 | CCCTCACACCAGTATTTATCCTTAACACTC |
| NM_000633 | ACCCTGGGTGCCTATCTGGGCCACAAGTGA |
| NM_005100 | CAGGAGGAGAACCGAAATATCGAACAGATT |
| NM_014207 | GACTATGATCTGCATGGGGCTCAGAGGCTG |
| NM_005574 | GCCTGGACCCTTCAGAGGAACCAGTGGATG |
| NM_001831 | GCAGGAATACCGCAAAAAGCACCGGGAGGA |
| NM_006187 | GCTGCATGGGACGGAATGGCATCCCCATCC |
| NM_058176 | GCCCTTCTAGGAAATGAGCTGGAGCCACTT |
| NM_002738 | ACCAGTCCTAACACCTCCTGACCAGGAAGT |
| NM_001774 | GTCTACAACCGGCTCGCTCGATACCGTTAG |
| NM_133378 | TGGCTCCCTCTTCGACCGTCTGGTCCTCAA |
| NM_152866 | CCTCACCAATAGAAAATGACAGCTCTCCTT |
| NM_006184 | TTCTCGAGCGGCTCCCTGAGGTTGAGGTGC |
| NM_003955 | TTCCTGGACCAGTACGATGCCCCGCTTTAA |
| NM_002162 | ACGTCTATGCAGCCGACAGAAGCAATGGGG |
| NM_000598 | TGTGGATAAGTATGGGCAGCCTCTCCCAGG |
| NM_006472 | ACCACCGACTTATACTGAGGTGGATCCCTG |
| NM_005998 | AGAAAGGCGATGACCAGAGCCGGCAAGGCG |
| NM_000884 | CTCCATTCGTATGAGAAGCGGCTTTTCTGA |
| NM_006636 | GTCTAAAGAGCTTGGGGTAGCCACTAATTA |
| NM_000269 | CTGGTAGATTACACGAGCTGTGCTCAGAAC |
| NM_004619 | AGATGACACTCTGTTCTTGAAAGTGGCCGT |
| NM_004972 | GGATCAAATAAGGGATAACATGGCTGGATG |
| NM_156039 | CCCAGGCGATCTGCATACTTTAAGGACCAG |
| NM_002467 | CTTGTTGCGGAAACGACGAGAACAGTTGAA |
| NM_002460 | CAGATCTATCCGCCATTCCTCTATTCAAGA |
| NM_001242 | TGCAGAGCCTTGTCGTTACAGCTGCCCCAG |
| NM_001877 | TCTCCTCCTGTGACTCGCTGCCCTAATCCA |
| NM_004513 | GATGGACCTGTCACGATTGTCATCAGGAGA |
| NM_007289 | AAACAGATGTGCACAGTCCAGGCAATTTCA |
| NM_018209 | GATGATGGCTGGGACAACCAGAACTGGTAG |
| NM_001092 | AGGTTCTGGAGGACGAAGATGTGTTTCTCC |
| NM_002046 | GTGGACCTCATGGCCCACATGGCCTCCAAG |
| NM_002166 | CACGGATATCAGCATCCTGTCCTTGCAGGC |
| NM_001882 | ATCGGGGAATTCTGTTTGTCTGGTCTTTGA |
| NM_000698 | AGCCAGTGGTTCGCCGGCACTGACGACTAC |
| NM_021603 | TGACTGGGTTGTCGATGGACGGTGGCGGCA |
| NM_001923 | TGGCAGCGGTATGAAGCGAGAGGCCACTGC |
| NM_031966 | TAGTTCAAGATTTAGCCAAGGCTGTGGCAA |
| NM_001987 | CAGATGAAATCATGAGTGGCCGAACAGACC |
| NM_002574 | GACAAACATGGGGAAGTGTGCCCAGCTGGC |
| NM_000034 | TCAAATCCAAGGGCGGTGTTGTGGGCATCA |
| NM_004044 | TGGCTCCCGGCCAGCTCGCCTTATTTAGTG |
| NM_003656 | TGTTGCTGTCGAGACTGCTGCGTGGAGCCG |
| NM_002168 | CCGTATTATCTGGCAGTTCATCAAGGAGAA |
| NM_007146 | TGCCTTTCTTGCCCCAAGCTTTGCCTACAT |

TABLE 27

Polynucleotide probe sequences
for preparation of a large 50mer nucleic acid array

| GenBank | |
|---|---|
| Accession | |
| Number of | |
| Target gene | Oligonucleotide sequences (5′ to 3′) |
| NM_000246 | GGCTTTCCCCAAACTGGTGCGGATCCTCACGGCCTTTTCCTCCCTGCAGC |
| NM_005175 | GGCTGGCATTGGAACCGTGTTTGGCAGCTTGATCATTGGCTATGCCAGGA |
| NM_006559 | TGTGGGGAAGATTCTTGGACCACAAGGGAATACAATCAAAAGACTGCAGG |
| NM_030666 | CTTTTCTTTATTCGGCATAATTCCTCAGGTAGCATCCTATTCTTGGGGAG |
| NM_003955 | CCTATGAGAAAGTCACCCAGCTGCCGGGGCCCATTCGGGAGTTCCTGGAC |
| NM_018209 | GCCCCTCGGAGGGCCACAGTTATCAGAACAGCGGTCTGGACCACTTCCAA |
| NM_001165 | CAACATTTGACTTGTGTAATTCCAATCCTGGATAGTCTACTAACTGCCGG |
| NM_014670 | GTCTGAGCTGACTCTGTTACTGAAGATTCAGGAGTATTGCTATGACAACA |
| NM_144578 | CACACCAGGACTCTATCCTACTCCCAGTAATCCTTTCCAAGTGCCTTCAG |
| NM_144628 | CGGTTTCGGGGACTTCTGCGGCCTGAAGATCGAACAAAAGATGTCCTGAC |
| NM_032263 | AAGATTCGGCCACTTACAACAGTCTCCTGCAAGCTTTGAGCAAAGAGAGG |
| NM_006579 | GGGCCACCCTCTCTACTTCTGGTTTTACTTTGTCTTCATGAATGCCCTGT |
| NM_000877 | TGGTCAGGGGACTTTACACAGGGACCACAGTCTGCAAAGACAAGGTTCTG |
| NM_002266 | CCAGAAACTACCTCTGAAGGCTACACTTTCCAAGTTCAGGATGGGGCTCC |
| NM_018462 | GCTCGGGTGACAAAAGGTGAGACACTCACTAGAACAGTGCCGTGCTGCTG |
| NM_002444 | CATGCTGAGAACATGCGACTGGGCCGAGACAAATACAAGACCCTGCGCCA |
| AB032991 | GGAAGGTATGGTGCTATCTGCGGATTTGGCCTTTCCTTGATCAAATGGAT |
| NM_004762 | ATCAGCAGGGACCCTTTCTACGAAATGCTCGCAGCACGGAAAAAGAAGGT |
| NM_000295 | TGACCACCGTGAAGGTCCCTATGATGAAGCGTTTAGGCATGTTTAACATC |
| NM_030674 | GCCTGCTCATCGAGTAATGGTGAAGGCCACTGAAACCCGCCGAGAAAAAG |
| NM_022743 | TGAGACTGGCTTTTGATATTATGAGAGTGACACATGGCAGAGAACACAGC |
| NM_003217 | CGTCCTTGTTGATACTCAACTCATTATTGAAAAGGCCGAACATGGAGATC |
| NM_022366 | GCGTTCCAAAGATTGTGCTTATAAATGGCTGTATGATGAAACCCTGGAAG |
| NM_014268 | CTAATGGACATCCTGTATGCTTCAGAAGAACACGAGGGCCACACAGAAGA |
| NM_003352 | CTCTTTGAGGGTCAGAGAATTGCTGATAATCATACTCCAAAAGAACTGGG |
| NM_001923 | GCAAACCTACAGTATGACGATGGCAGCGGTATGAAGCGAGAGGCCACTGC |
| NM_033503 | TGAGGAACCCCAGCGACTCTTTTATGGCAATGCTGGCTATCGGCTTCCTC |
| NM_001776 | CCCTGGTCCTTTTCACAGTGGCCATCATAGGCTTGCTTATCTTTCACAAG |
| NM_178155 | AAAGTTCGAGAGAAGATAGAAACGGTCAAGTACCCCACATATCCTGAGGC |
| XM_088315 | CCAGTACTACCGGCCCCTGCATGATGAGTACTGTTTCTACAATGGCAAAA |
| AK024458 | TTCCCGGTGAGGTCAGGCCTCGTATTACATAAAAATACCTGTGCTGGGAC |
| NM_014397 | TGTCACCGGAGAGGATCCATGAGAACGGCTACAACTTCAAGTCCGACATC |
| NM_005969 | TTTTTCCGTGAGCGGATAGTCCCGCGGGCTGTGCTGTACTTCACTGGGGA |
| NM_002221 | CGGGAGGATGGCTACCTCTCGGGGCTCAATAACCTCGTCGACATCCTGAC |
| NM_013314 | TAGATCAACCAAGCCAAATTCCTCAACGCCCGCCTCTCCTCCAGGAACAG |
| NM_004844 | TGGGATGATGTTCCCAGTGTTGGGCCCTCGAAGTGAATGCAGCGGGGCCT |
| NM_001759 | CAGCAGTACCGTCAGGACCAACGTGACGGATCCAAGTCGGAGGATGAACT |
| NM_001987 | AAAATATTCCGGATAGTGGATCCCAACGGACTGGCTCGACTGTGGGGAAA |
| NM_002827 | CTACGGTCCTCACGGCCGGCGCTTACCTCTGCTACAGGTTCCTGTTCAAC |
| NM_004513 | TGGGTGGCACTGCCATGCAGGGCCTCACACGGTTTGAAGCCTGGAACATC |
| NM_001237 | CTACCTCAAAGCACCACAGCATGCACAACAGTCAATAAGAGAAAAGTACA |
| NM_172164 | GGAGGTCAGGAGAAGCAGGGAGAGGTAATTGTGAGCATAGAGGAGAAGCC |
| NM_000424 | CCAGTCAAGTGTGTCCTTCCGGAGCGGGGGCAGTCGTAGCTTCAGCACCG |
| AB037771 | GCCAACCAGAACTAGACTCTATTTCTACCTGTCCAAATGAGACAGTTTCA |
| NM_025113 | AGCCCATTTTCAATTTGCTGAGCATCGGCCAAAGCCTGTATGCGAAAGCC |
| NM_032873 | ACCAATCCTTCCTCTTACCCATGGACCAACTGGGGGCTTCAACTGGAGAG |
| NM_023037 | TCCTCACTACTTTTCTTCCAGACTCCAGTGTTTCTGGCACTAGTCTCTGA |
| AF509494 | AAGAAGAAAGGCGACAGCAACTTCGTAGGAAGGTAGTTGAAATTCTTCCA |
| NM_004556 | AGGCTGGTGCCCAGGTAGATGCCCGCATGCTGAACGGGTGCACACCCCTG |
| NM_002984 | CAGCCAGCTGTGGTATTCCAAACCAAAAGAAGCAAGCAAGTCTGTGCTGA |
| BC025340 | CGCAGCATGGAGTCTGCTCTCAGCTGTTGGGGAATTACCGATGCCTTTGA |
| NM_030926 | CCCCTCGCAACTTCTGGGAGCTCCTCATGAACGTGAAGAGGGGGACCTAC |
| AF331856 | GGTATTTGGTTTCCCAGTCCACTATACTGACGTCTCCAACATGAGCCGCT |
| NM_000483 | CCGCTGTAGATGAGAAACTCAGGGACTTGTACAGCAAAAGCACAGCAGCC |
| NM_001888 | CAGCCCACTGTGAGAAGACCACGGTGTTCAAGTCTTTGGGAATGGCAGTG |
| NM_003804 | AGCTATCTTTGATAATACCACTAGTCTGACGGATAAACACCTGGACCCAA |
| AB018263 | TCAGGACCACCCTGCTGCTTAACTCCACGCTCACTGCCTCGGAGGTCTGA |
| NM_006230 | CCGGACACTCTAGGTTGTTACCCCTTCTACAAAACTGACCCGTTCATCTT |
| NM_006475 | GCAGTCTTCAGCCTATTATCAAAACTGAAGGACCCACACTAACAAAAGTC |
| NM_001786 | AATCCTACAGGGGATTGTGTTTTGTCACTCTAGAAGAGTTCTTCACAGAG |
| NM_031942 | CGGCAGCGAGATGGACGGTGTGCGACTGGGGTCCTTGTGTATTTAGCCAA |
| NM_001640 | CGCATGGAGAACATTCGATTCTGCCGCCAATACCTGGTGTTCCATGACGG |
| NM_002483 | GTGCTCCTGTCCTCTCAGCTGTGGCCACCGTCGGCATCACGATTGGAGTG |
| NM_003915 | CACACCCATCCAGGTGCAATGCTCCGATTATGACAGTGACGGGTCACATG |
| NM_018842 | AACTCCGCCCGACTGTGACGAATGATCGCTCGGCACCCATCATTCGATGA |
| NM_147180 | CCACAATGGGAAACGAGGCCAGTTACCCGGCGGAGATGTGCTCCCACTTT |
| NM_004924 | AACCCCTACACCACCGTCACCCCGCAAATCATCAACTCCAAGTGGGAGAA |
| NM_002276 | CCTGCTCGAGGGACAGGAAGATCACTACAACAATTTGTCTGCCTCCAAGG |
| NM_002695 | GGTGAAGATCATCCGGCCCAGTGAGACGGCTGGCAGGTACATCACCTACC |
| NM_003480 | CTGCCCCCTAGGAGACTCCGTCGCTCCAATTACTTCCGACTTCCTCCCTG |
| NM_002023 | CAACAATGTCTACACCGTCCCCGATAGCTACTTCCGGGGGGCGCCCAAGC |
| NM_006216 | GCCGCTGAAAGTTCTTGGCATTACTGACATGTTTGATTCATCAAAGGCAA |
| NM_021813 | GAGAAACTGTTGTCAGAGAGGAATCAACTGAAAGCATGCATGGGGGAACT |
| NM_003651 | GCAGGTGAAGCACCAACTGAGAACCCTGCTCCACCCACCCAGCAGAGCAG |
| NM_006565 | GAGAGGACGACCCCCTGGCAGAACCAACCAGCCCAAACAGAACCAGCCAA |
| NM_005537 | GGGGTGGAGGGTGGACGAGTTGATTTGAACGTCTTCGGGTCGCTCGGCCT |
| NM_004235 | CGTGGCCCCGGAAAAGGACCGCCACCCACACTTGTGATTACGCGGGCTGC |
| NM_006235 | AAGAGGATAGCGACGCCTATGCGCTTAACCACACTCTCTCTGTGGAAGGC |
| NM_016269 | CTATCAACCAGATTCTTGGCAGAAGGTGGCATGCCCTCTCCCGTGAAGAG |
| NM_006164 | TCAGCACCTTATATCTCGAAGTTTTCAGCATGCTACGTGATGAAGATGGA |
| NM_003222 | CGAACATACAGAACTGCTTGTCTCATTTCAGCCTGATTACCCACGGGTTT |
| NM_000700 | GCCATAAGGCATTGATCAGGATTATGGTTTCCCGTTCTGAAATTGACATG |
| NM_019846 | GGGCACATCAGGGGAAACACGAAACATACGGCCATAAAACTCCTTATTAG |
| NM_032738 | CAGAAACAGCATCTGTTGTGGCTATCACAGTCCAAGAACTGTTTCCAGCG |
| BC019046 | TCACCATCTTCATCACACTCTTCCTGTTAAGCGTGTGCTACAGTGCCACC |
| NM_016732 | CATCTTTGACTATGATTACTACCGGGACGACTTCTACGACAGGCTCTTCG |
| NM_002189 | GAGCGCTGTGTCTCTCCTGGCATGCTACCTCAAGTCAAGGCAAACTCCCC |
| NM_004827 | AGGGCATCGATCTCTCACCCTGGGGCTTGTGGAAGAATCACGTGGCCTTG |
| NM_001830 | TTGCCCAGAGGAGGGAACTGATTCTCGCAATAAATAACGCCAGACAGAGG |
| NM_020944 | CAAACAGGGCACAGGACTAAGGACAGGGCCTATGTTTGGACCAAAGGAAG |
| NM_017784 | AGGAACGGAAGCGCGAGAACCTCCGCACACCATGGAAGCCCAAATATTTT |
| NM_002933 | TGTGGCCTGTGAAGGGAGCCCATATGTGCCAGTCCACTTTGATGCTTCTG |
| NM_022829 | CCTTCCCGGACTGGGCTGATATGTACTCGGTCAATGTCACAGCATTGCCA |
| NM_006399 | CCCACGCATTCCACCAACCTCATGTCAGCTCCCCGCGCTTCCAGCCCTGA |
| NM_002893 | CCTTCGAATCTCATAAAGATGAAATTTTCCAGGTCCACTGGTCTCCACAT |
| NM_003339 | AGATGATCCTTTAGTGCCTGAGATTGCTCGGATCTACAAAACAGATAGAG |
| NM_005277 | AATCTCTGCTTGGACCTTCGTCAGTTTGGAATTGTGACAATTGGAGAGGA |
| NM_006820 | CAAAGCCGGGTCATGAATGTCCATAAAATGCTAGGCATTCCTATTTCCAA |
| NM_024728 | CCCATGGCCCCTTCCTTCATCTTCAAGGATCAGCAAGGGTATTACACAAT |
| NM_025263 | GCTTTCACAAGGAACAGAGAAACCCTCGAAGGCTCAAAAGCTGGTCTCTT |
| BC046632 | AGCAGTGCCGGTGCATGTCCGTGAACCTGAGCGACTCGGACAAGCAGTGA |
| NM_024713 | CAGGCCACCTGCCTGAAAAATTACACCATGATAGTCGAACATATTTGGTT |
| NM_014365 | TTGACAGCCTCTTGGCCCGACTGGGCTCTGCCTCGTCTCTCCTCCGCCTG |
| NM_002947 | CGCGCATCAACGCCGGCATGCTAGCTCAATTCATCGACAAGCCTGTCTGC |
| AK092000 | ATCGAGCATGCTATTCCAGTGTACTGAACATACTGTATACCTCGTGTTAG |
| NM_022436 | GCTCAAATGTTTCTGTGACAACTAATCCAATGTGTGCCTTCACTCAAGGA |
| NM_002967 | TGATGAACCGAGGAGGAATGTCAGGGCGCGGCAGCTTTGCCCCAGGCGGG |
| BX647445 | ACTGTTGCCCTGGCTGTATTCATAAGATTCCAGCTCCTTCAGGTGTTTGA |
| BC022095 | CATGCGCACGGGGTTCCTAGCTCCCACGTCTATGTTCACATTTGTGGTCC |
| NM_006808 | GCCCTGTTCCAGTATTGGTTATGAGTCTTCTGTTCATCGCTTCTGTATTT |
| NM_031305 | CAGACTGTCCACCTATGATAATGTCCATCAACAGTTCTCCATGATGAACC |
| NM_014792 | CCGGTGAGCTTGTGGTGTGGGTTTTCAGGCTGTATCCTTCTACCTCCTGA |
| NM_006304 | GTAGAGGATGACTTCTCTAATCAGTTACGAGCTGAACTAGAGAAACATGG |
| NM_014686 | TGTGAACCAGGATAACAAAACCAAAACGTGGCCACCCAAAGCACCCTGGC |
| AB029034 | CAGGCAAACCGCAGCACCACACCTATGGCCCCCGGTGTCTTCTTGACCCA |
| BQ722784 | ATCGGTCTTTCTCTTTCAATCCTCACCCGGGATATTTGACCCCTTCCATG |
| NM_198243 | CTGTGTGCACGGATGTTATATAATTACGGAGCAGACACGAACACACGGAA |
| NM_016570 | GCACATGCCATTCTGGCAGTTTTTTGTAAGACTCTGTGGTATTGTTGGAG |
| BC001077 | CCACTGCCCTCTAAGACCTTGGAAGGGGAAACACCAGAAGGTGTGGGTGC |
| NM_138379 | ACATTGGAGATAGTAAAAATGTCCTCAATGACGTGCAGCATGGAAGGGAA |
| NM_024319 | ATGTTCGGCTCCAGTCGTGGAGGCGTGCGCGGCGGGCAGGACCAGTTCAA |
| NM_015670 | CCTATTTTGACTCGCAGCGTACCCTAAACCGCCGCTGCCCTAAGCATATT |
| NM_015436 | CCTTGCTGTGACAAGCTTTATACTTGCCGCTTGTGTCATGATAACAATGA |
| NM_001240 | AATCCTCGGTCTGGTGGAATCTCCTCGAGATCTGGCAATACAGACAAACC |
| NM_001261 | ATGTTTTGGTCAAGTTCACGCTGTCTGAGATCAAGAGGGTGATGCAGATG |
| NM_003095 | GCAAATACAGAAGAATACATAGATGGAGCTTTGTCTGGACATCTGGGTGA |
| NM_021102 | TGCACCGCCAACGCAGTCACTGGGCCTTGCCGTGCATCCTTCCCACGCTG |
| NM_006819 | GCTACCAGCGCTGTATGATGGCGCAGTACAACCGGCACGACAGCCCCGAA |
| NM_032966 | AGTTCCGCAGTGACCTGTCGCGGCTCCTGACGAAGCTGGGCTGTACCGGC |
| NM_006284 | ATTCGGCTCATCTCCTTAGCTGCCCAGAAATTCATCTCAGATATTGCCAA |
| NM_003199 | TTCACCTCCTGTGAGCAGTGGGAAAAATGGACCAACTTCTTTGGCAAGTG |
| NM_030752 | ACCTCGAGACAACAAACAAGCAGGGGTGTTTGAACCAACCATAGTTAAAG |
| NM_003299 | AGAATGCTTCGCCTCAGTTTGAACATTGACCCTGATGCAAAGGTGGAAGA |
| NM_006758 | ATGGACAGCCGATCCACGCCGAGCTGTCACCCGTGACGGACTTCAGAGAA |
| NM_194259 | AGTACGAGAAAAGGGTCCGAGCACAAGCCAAGAAGTTTGCGCCCTCATAA |
| NM_003366 | CAATGGCAGCAAGTGGAAATTTGGGACATACACCTTTTGTTGATGAGTTG |
| NM_006004 | ATGCGAGGGACCATTGCGTGGCCCACAAACTCTTTAACAACTTGAAATAA |
| NM_003374 | TGGACAGCAGGAAACAGTAACACGCGCTTCGGAATAGCAGCCAAGTATCA |
| NM_006761 | ATTTTTCCGTATTCTACTACGAAATTCTTAATTCCCCTGACCGTGCCTGC |
| NM_005781 | AGCTCTTCGGGCTGGGTCTGCGGCCCAGAGGGGAGTGCCACAAAGTGCTG |
| NM_005243 | TTAAAAAGTACCCGTACTCAGTACTCAGCCGGCAGCATAATGAAAAGTGG |
| NM_003902 | GCTGAGTATTATAGACAACAAGCAGCCTATTATGCCCAGACAAGTCCCCA |
| NM_006769 | GAATCGCCTGGTCCCGGGAGATCGGTTTCACTACATCAATGGCAGTTTAT |
| NM_004689 | GCTGGCCGGGCCTGCGAGAGCTGTTACACCACACAGTCTTACCAGTGGTA |
| NM_006875 | TTCCCAGCCCATGTCTCCCCAGACTGCTGTGCCCTAATCCGCCGGTGCCT |
| NM_002953 | CTGGCAGGATATACTCCATTTGCCAACGGTCCCAGTGACACACCAGAGGA |
| NM_004965 | AGCTAAACCTCCTGCAAAAGTGGAAGCGAAGCCGAAAAAGGCAGCAGCGA |
| NM_000592 | GCAGCCTGTGCCCAGCTCAACGACTTCCTCCAGGAGTATGGCACTCAGGG |
| NM_178014 | CGGCCGATACCTCACCGTGGCTGCTGTCTTCCGTGGTCGGATGTCCATGA |
| NM_005742 | TCAGGGAGCTCTCTTTTGGGCGTGGCTCCACGGCACCTGTAGGAGGCGGG |
| NM_001901 | TGGGCCTGCCCTCGCGGCTTACCGACTGGAAGACACGTTTGGCCCAGACC |
| NM_004907 | GGGACGTTGGACTGGTCCCGAGCAAGAAAGCCCGTCTGGAAGAAAAGGAA |
| NM_003070 | GCGGAGGGAAGATGCCCGGAACCCGAAACGGAAGCCCCGTTTAATGGAGG |
| NM_001255 | ATGAGACCCTGAGGCTATGGCGCTGTTTTGAGTTGGACCCTGCGCGGCGG |
| NM_002417 | CCTACGGAAACTGAAATTCACAATGAGCCATTTTTAACTCTGTGGCTCAC |
| NM_006209 | ATACATAGGATGGATCATTATGCTGCGGAAACTCGTCAGGACAAAATGAC |
| NM_002162 | AGAGCACCTATCTGCCCCTCACGTCTATGCAGCCGACAGAAGCAATGGGG |
| NM_000587 | AGACGATGTCTGAGTGTGAGGCGGGCGCTCTGAGATGCAGAGGGCAGAGC |
| NM_031243 | GAGGAGGATCTGATGGATATGGCAGTGGACGTGGATTTGGGGATGGCTAT |
| NM_003017 | CACTCCGAAGTGTGTGGGTTGCTAGAAACCCACCCGGCTTTGCTTTTGTT |
| NM_004994 | AACCAGGTGGACCAAGTGGGCTACGTGACCTATGACATCCTGCAGTGCCC |
| NM_002966 | CGCTGGGGATAAAGGCTACTTAACAAAGGAGGACCTGAGAGTACTCATGG |
| NM_001345 | CCGGGCTGCATTGTGTATGGTGCCACCTAGAGATCCACGATGACTGCCTG |
| NM_002497 | GAACATCATGAGGAGTGAGAATTCTGAGAGTCAGCTCACATCTAAGTCCA |
| NM_000545 | GCCTCACGCCCACCAAGCAGGTCTTCACCTCAGACACTGAGGCCTCCAGT |
| NM_006184 | GCCGAGCAGGATCCCAATGTACAGGTGGATCATCTGAATCTCCTGAAACA |
| NM_000598 | AGTTCCTCAATGTGCTGAGTCCCAGGGGTGTACACATTCCCAACTGTGAC |
| NM_006152 | AGGACTCATGGACGTCTCTAGAACATATCTTGTGGCCATTTACCAGACTC |
| NM_006274 | GACCAGCCCTGGGTAGAACGCATCATCCAGAGACTGCAGAGGACCTCAGC |
| NM_001428 | CATCCTGCCAGTCCCGGCGTTCAATGTCATCAATGGCGGTTCTCATGCTG |
| NM_002415 | TGCACAGCATCGGCAAGATCGGCGGCGCGCAGAACCGCTCCTACAGCAAG |
| NM_003234 | ATGAAACGCTGTTCAGAAACCAGTTGGCTCTAGCTACTTGGACTATTCAG |
| NM_001728 | TCGTGAGTTCCTCGCAGGGCCGGTCAGAGCTACACATTGAGAACCTGAAC |
| NM_002460 | GCAATCCAGAAGATTACCACAGATCTATCCGCCATTCCTCTATTCAAGAA |
| NM_003403 | CCCTCATAAAGGCTGCACAAAGATGTTCAGGGATAACTCGGCCATGAGAA |
| NM_001344 | TGGGGAGTTTCATCCTAGCGGTTTGCCTGAGAATACAGATCAACCCACAG |
| NM_002648 | CTGTATGATATGGTGTGTGGAGATATTCCTTTCGAGCATGACGAAGAGAT |
| NM_006585 | CTTACCTGGGAAAATATTGGGCTATCAAACTCGCTACTAATGCTGCAGTC |
| NM_001826 | TATTCGGACAAATACGACGACGAGGAGTTTGAGTATCGACATGTCATGCT |
| NM_002466 | TACTCCATGGACAACACTCCCCACACGCCAACCCCGTTCAAGAACGCCCT |
| NM_005582 | GCAAACCCGCCATCTCTAAGGGGAGTTAAGCTATCTGATGTCAAGCTTTC |
| NM_001831 | TGACTCTGATCCCATCACTGTGACGGTCCCTGTAGAAGTCTCCAGGAAGA |
| NM_000038 | CAGGGCCAGTGCAGCACTCCACAACATCATTCAGTCACAGCCTGATGACA |
| NM_001226 | GAGACAATCTTACCCGCAGGTTTTCAGATCTAGGATTTGAAGTGAAATGC |
| NM_005190 | GAGAGCAGGGTCCAAATGGAAGTCAGAACTCTAGCTACAGCCAATCTTAA |
| NM_001795 | ACGTGGATTACGACTTCCTTAACGACTGGGGACCCAGGTTTAAGATGCTG |
| NM_030582 | GGGCACCACTCACGGGGCCCTCAGTGCCACCACCATCTTCAGAGCGCATC |
| NM_001848 | CGACAACCTGAGGGACAGGTACTACCGCTGTGACCGAAACCTGGTGTGGA |
| NM_004378 | CGTTTGGCGCCGATGACGTGGTCTGCACCAGAATTTATGTCCGGGAATGA |
| NM_004380 | GCCACGTTGCCTCCGCACAGCCGTTTACCATGAGATCCTTATTGGATATT |
| NM_005215 | CCAACAGAGGATTCAGCCAATGTGTATGAACAGGATGATCTGAGTGAACA |
| NM_004948 | AGAGGGACTGGAGTTTCTAGATCACCTGGAACCCAAATTTAGGACATTAG |
| NM_001397 | AGGCGCTGAGGGAGTCCGTGCTGCATTTGGCCTTGCAGATGTCGACGTAC |
| NM_004448 | GCCTCTTAGACCATGTCCGGGAAAACCGCGGACGCCTGGGCTCCCAGGAC |
| NM_001982 | AAAAACTCTACGTAGCTTAGAGGCTACAGACTCTGCCTTTGATAACCCTG |
| NM_006137 | TCGGCGGCATGTGTGGTGTACGAGGACATGTCGCACAGCCGCTGCAACAC |
| NM_005245 | ATGCCCCTTACCCGCCAGGGTATCAAAGACACTTCGAGGCGCCCGCTGTC |
| NM_004119 | AGATGGATTTGGGGCTACTCTCTCCGCAGGCTCAGGTCGAAGATTCGTAG |
| NM_182925 | GCCGCCAGGTATTACAACTGGGTGTCCTTTCCCGGGTGCCTGGCCAGAGG |
| NM_000629 | CTCTCCCGTTTGTCATTTATGCTGCGAAAGTCTTCTTGAGATGCATCAAT |
| NM_001552 | GGTGCCTGCAGAAGCACTTCGCCAAAATTCGAGACCGGAGCACCAGTGGG |
| NM_000572 | TAAGGGTTACCTGGGTTGCCAAGCCTTGTCTGAGATGATCCAGTTTTACC |
| NM_000640 | GGTCTGCTTTTGCGTAAGCCAAACACCTACCCAAAAATGATTCCAGAATT |
| NM_000564 | GAAGTCATCTGTTATATAGAGAAGCCTGGAGTTGAGACCCTGGAGGATTC |
| NM_176786 | GCATGTTGCTCCCTTCTGTCCTCAGCAAGGCTCGGTCCTGGACATTCTAG |
| NM_000213 | GTAAGGACCCAGGAACTAGGCCTGGCCGGAGATGTGGCTGAACGAGGTCT |
| NM_001967 | TTGGAGATTGAGTTCAAGGAGACCCAAGCACTAGTATTGGCCCCCACCAG |
| NM_004933 | GGACGACCTTCGAGACAATGTCCTCAACTACGATGAGCAAGGAGGCGGGG |
| NM_000224 | CTTGGTGATGCCTTGGACAGCAGCAACTCCATGCAAACCATCCAAAAGAC |
| NM_002758 | GAATTCCAAAGAACGGCCTACATACCCAGAGCTAATGCAACATCCATTTT |
| NM_002412 | CAGCGGAGCCGTGGGCAACTACTCCGGAGGACTGGCCGTGAAGGAATGGC |
| NM_004530 | TGAAGAGCGTGAAGTTTGGAAGCATCAAATCCGACTGGCTAGGCTGCTGA |
| NM_002422 | TTTCCCTCCAACCGTGAGGAAAATCGATGCAGCCATTTCTGATAAGGAAA |
| BC063294 | ACAAGCCGCATACGCACCCAGAGCTTTTCCTTGCAGGAGCGTCAGTTGCG |
| NM_004557 | CGGCCCAACCCTGCGATAATGCGAGGAAGATACGGAGTGGCTGCCGGGCG |
| NM_002514 | TCATTGGGACCTGCACCTGTCACACCAACTGTCCTAAGAACAATGAGGCC |
| NM_000300 | ACCAGTACTATTCCAATAAACACTGCAGAGGGAGCACCCCTCGTTGCTGA |
| NM_001276 | AGTGGGTAGGATACGACGACCAGGAAAGCGTCAAAAGCAAGGTGCAGTAC |
| NM_000535 | CACTGAAAGGGCTAAACTGATTTCCTTGCCAACTAGTAAAAACTGGACCT |
| NM_015869 | GACAGATTGTCACGGAACACGTGCAGCTACTGCAGGTGATCAAGAAGACG |
| NM_002805 | GAACGAACTAAATGCTAAAGTTCGCCTATTGCGGGAGGAGCTACAGCTGC |
| NM_000314 | GGAAGTCTATGTGATCAAGAAATCGATAGCATTTGCAGTATAGAGCGTGC |
| NM_002868 | AGGGGTGCCCAAGCTGCAATCGTGGTTTACGACATTACTAATCAGGAAAC |
| NM_002901 | GTTGGAAGCCAAGCTACCAATTACGGGGAAGATCTCACAAAAAATCATGA |
| NM_003821 | GTCTTCAGCCTTACCCGGAAATACTTGTGGTTTCTAGATCACCATCTTTA |
| NM_005617 | CAGAGCCCTTGCCCGCTCGGGTATGAAGATCGGGCGGATTGAGGATGTCA |
| NM_014624 | CCTTCCTGGGGGCCTTGGCTTTGATCTACAATGAAGCCCTCAAGGGCTGA |
| XM_044622 | CCAGGAGATTGAAGGACCTAGTGTGAGCATAATGGAAAAAACACAATCAC |
| NM_000602 | ATGTTCAGACAGTTTCAGGCTGACTTCACGAGTCTTTCAGACCAAGAGCC |
| NM_003012 | AGAAAATGAAAAACCATGAGTGCCCCACCTTTCAGTCCGTGTTTAAGTGA |
| AF311912 | ACAAGCTGAACGGTGTGTCCGAAAGGGACCTGAAGAAATCGGTGCTGTGG |
| NM_139276 | TCACCTTTGACATGGAGTTGACCTCGGAGTGCGCTACCTCCCCCATGTGA |
| NM_003220 | GCCAGGGACTTTGGGTACGTGTGCGAAACCGAATTTCCTGCCAAAGCAGT |
| NM_003225 | TGTGCAAATAAGGGCTGCTGTTTCGACGACACCGTTCGTGGGGTCCCCTG |
| NM_003842 | AGCACAGTGGGGAAGCCCCAGCTGTGGAGGAGACGGTGACCTCCAGCCCA |
| NM_000546 | AGTATTTGGATGACAGAAACACTTTTCGACATAGTGTGGTGGTGCCCTAT |
| NM_138764 | GGCCTTCTGGAGCAGGTCACAGTGGTGCCCTCTCCCCATCTTCAGATCAT |
| NM_001328 | AAACCGTCAAGCCCGAGGCGGATAGAGACCACGCCAGTGACCAGTTGTAG |
| NM_058197 | ATTGGAATCAGGTAGCGCTTCGATTCTCCGGAAAAAGGGGAGGCTTCCTG |
| NM_000594 | CCCCCAGAGGGAAGAGTCCCCCAGGGACCTCTCTCTAATCAGCCCTCTGG |
| NM_003810 | TAGACATGGACCATGAAGCCAGTTTTTTCGGGGCCTTTTTAGTTGGCTAA |
| NM_001798 | ACCCTTTCTTCCAGGATGTGACCAAGCCAGTACCCCATCTTCGACTCTGA |
| NM_002609 | AAACCCGAGGTTGCTGACGAGGGCCCACTGGAGGGTTCCCCCAGCCTAGC |
| NM_003005 | CTAGGAACATATGGAGTTTTTACAAACGCTGCATTTGACCCGAGTCCTTA |
| NM_002985 | GCAGTCGTCTTTGTCACCCGAAAGAACCGCCAAGTGTGTGCCAACCCAGA |
| NM_000964 | AGCATATGTTCGGGGATTTGGTGTGCAGTTGGAAATTGGCAGCCATTGAG |
| NM_024408 | CAGGACGGGCAGGTAGCTCAGACCATTCTCCCAGCCTATCATCCTTTCCC |
| NM_001465 | GATACCTCTGATTTCCCTGTTTCATCAGCAGAGATGAGTCAAGGAACTAA |
| NM_001618 | GGTGTAGACGTTCCTCTTGGGACCGGGATTTCATCTGGTGTGATAGACAC |
| NM_078487 | GTGCGACAGCTCCTGGAAGCCGGCGCGGATCCCAACGGAGTCAACCGTTT |
| NM_004322 | GGACTTCCTCGCCCGAAGAGCGCGGGCACAGCAACGCAGATGCGGCAAAG |
| NM_021960 | AAGGACAAAACGGGACTGGCTAGTTAAACAAAGAGGCTGGGATGGGTTTG |
| NM_138578 | AGCTTTGAACAGGATACTTTTGTGGAACTCTATGGGAACAATGCAGCAGC |
| NM_004383 | CGTCCAGAAGCGGGAGGGCGTGAAGGCGGGTACCAAACTCAGCCTCATGC |
| NM_015583 | GCCAGGTAGGTTGGAATGTTCAGTGATGTTCTGTAGTCATATCAGACAGT |
| XM_373407 | CCGCCGCAACCTCAGCTACACCTGCCGGTCCAACCGTGACTGCCAGATCG |
| NM_016734 | TTGTGACAGGCCGTGACTTGGCGAGCACGACCCTCCCCGGGTACCCTCCA |
| NM_000808 | CAACAGTGTCAGCAAGGTTGACAAAATTTCCCGCATCATCTTTCCTGTGC |
| NM_002964 | TCTGGTGATAAAGATGGGCTGGCAGCCCACAAAAAAAGCCATGAAGAAAG |
| NM_002965 | AGATGCACGAGGGTGACGAGGGCCCTGGCCACCACCATAAGCCAGGCCTC |
| NM_001250 | CCAGGAGATCAATTTTCCCGACGATCTTCCTGGCTCCAACACTGCTGCTC |
| NM_003407 | TCCTGTCGCTGAGCCCTGACGTGCCCGTGCCATCCGACCATGGAGGGACT |
| NM_003674 | GGAGAAGCCCCTACCCTGTGAGCCGGAGCTCATGCCGACCTTTCCCCACC |
| NM_078467 | GCATGACAGATTTCTACCACTCCAAACGCCGGCTGATCTTCTCCAAGAGG |
| NM_006145 | CCCGAAAGGATTCCCCAGACATCAAGAACCGTACTTGAGCAGGTTCTTCC |
| NM_000122 | AGGCGCCCAGCAAACATGTACACCCGCTCTTCAAGCGCTTTAGGAAATGA |
| NM_006712 | TGCCGCTACCCTTCGAGGAACTGGAGTCCCAGAGAGGCCTGCCCCAGCTC |
| NM_000175 | TGCAGTGGCGAAGCACTTTGTTGCCCTGTCTACTAACACAACCAAAGTGA |
| NM_000162 | CCCAGACCAAGATCCCAACTCCCACAACCCAACTCCCAGGTAGAGCAGAT |
| NM_000852 | CCTCCCCTGAGTACGTGAACCTCCCCATCAATGGCAACGGGAAACAGTGA |
| NM_005526 | TAGAGGAGGCGAGTCCCGGGCGCCCATCTTCCGTGGACACCCTCTTGTCC |
| NM_001540 | TGGCTACATCTCCCGGTGCTTCACGCGGAAATACACGCTGCCCCCCGGTG |
| NM_002432 | GAACAGTTGACCGCAAGCTGAAACTGGTGTGTGGAAGTCACAGCTTCATC |
| NM_002505 | CATCCGCCTCAGGCCAGCAAGTCCAGACCCTCCAGGTAGTCCAAGGGCAG |
| NM_002511 | GGGAGGAAGTCCTATCAAGAGAGAGGAACCAGCTACCTACTCAGCTCTTC |
| NM_006170 | CTCTGTGAGGATGCCTTCTCTACTGTGCATACCCATGAAATTTAATACAC |
| NM_002910 | TTTCAAAGGCTGCTTCCACGTGCCGCGGTGCCTAGCCATGTGCGAGGAGA |
| NM_005281 | CTGCCTGCCTCCCACTATGTGGCCACCCGCAAGGGCATTGCCACACTGGC |
| NM_005981 | GATTTCGGAATCAGAAGGATCCTAGAGCCAACCCCAGTGCCTTTCTATGA |
| NM_003410 | GTTCTTCTGGAATGACCATGGACACAGAGTCGGAAATTGATCCTTGTAAA |
| NM_007146 | AATGAGACCTGTAGAGAGCATGCCTTTCTTGCCCCAAGCTTTGCCTACAT |
| NM_002419 | CTCGCCCCTTCGCAGCCGCATTGATCCCTGGAGCTTTGTGTCAGCTGGGC |
| NM_001197 | GGAGAACATAATGAGGTTCTGGAGATCCCCGAACCCCGGGTCCTGGGTGT |
| NM_000418 | GGACAGGGAGCCACCTCGCAGTCCGCAGAGCTCACATCTCCCAAGCAGCT |
| NM_006254 | GTGTGGACACGCCACATTATCCCCGCTGGATCACCAAGGAGTCCAAGGAC |
| NM_002166 | TTGGACCTGCAGATCGCCCTGGACTCGCATCCCACTATTGTCAGCCTGCA |
| NM_033306 | TTGACCATCTGCCTCCGAGGAATGGAGCTGACTTTGACATCACAGGGATG |
| NM_020250 | GGGCGGGGTTTAAGAGGACGACAGGTGTCTGTATTGCTCTACCTACCTAA |
| NM_002228 | TCCTCCCGTCCGAGAGCGGACCTTATGGCTACAGTAACCCCAAGATCCTG |
| NM_001654 | GCAAAATCTCCAGCAACTGCCCCAAGGCCATGCGGCGCCTGCTGTCTGAC |
| NM_004047 | GCCGTGGGAGTCTGCTACACCATTTTTGATTTGGGCTTCCGCTTTGATGT |
| NM_000729 | CCGGAAAGCTCCTTCTGGACGAATGTCCATCGTTAAGAACCTGCAGAACC |
| NM_006273 | CAGAAGGACCACCAGTAGCCACTGTCCCCGGGAAGCTGTAATCTTCAAGA |
| NM_001256 | ATCCCAGGAGAGCAGCATGACAGATGCGGATGACACACAACTTCATGCAG |
| NM_004073 | TTGTGGCCCGAAATCGTAGTGCTTGTACTTACCTCGCTTCCCACCTTCGG |
| NM_006268 | CGTGGCTACCACATGTACTGTCTCACCCCGTCCATGTCTGAGCCCCCTGA |
| NM_002104 | GAAATACCAGACTTGGATCAAAAGCAACCTTGTCCCGCCTCATACAAATT |
| AK127132 | TTTAAATATGCTCTGGTCTGGGGTCTCTCTGTGAAACACAATCCTCAGAA |
| NM_004422 | GGGAAGCAAGTGGGACTAGCGATGGGGGCCCTCCTCCATCCAGAGGCTCA |
| NM_006014 | TTGGAGGCGGAAATCGCCCATGGGTCCCTGGCACCAGATGCCGAGCCCCA |
| NM_003824 | CAGACACCAAGATCGACAGCATCGAGGACAGATACCCCCGCAACCTGACA |
| NM_002055 | GAAGGCCACCTCAAGAGGAACATCGTGGTGAAGACCGTGGAGATGCGGGA |
| NM_005313 | TGTGCCTTCTCCATATGAAGTCAGAGGTTTTCCTACCATATACTTCTCTC |
| NM_001513 | ATCTCACCCCCTACCCTACCATCAGCTCCATCAACAAGAGGCTGCTGGTC |
| NM_003883 | TGGCCAAGACCGTGGCCTATTTCTACGACCCCGACGTGGGCAACTTCCAC |
| NM_005339 | GACCTCCAGACACACCATATGAAGGAGGAAGATACCAACTAGAGATAAAA |
| NM_003549 | TCAGGGCCCCCTTCAAAAAGCCTGCTGGAGACACAATATCAAAATGTGAG |
| NM_004763 | ATGCTCTCTACTTAATAATCCGGATGGTGTGTTACGATGACGGTCTGGGG |
| NM_002301 | GCGGAATGGTGTCTCAGATGTTGTGAAAATTAACTTGAATTCTGAGGAGG |
| NM_004993 | GCTTCAGGCAGCTGTGACCATGTCTTTAGAAACTGTCAGAAATGATTTGA |
| NM_002790 | AGGAGAAGCTGAATGCAACAAACATTGAGCTAGCCACAGTGCAGCCTGGC |
| NM_002971 | CAAACTGAAGGACAATTCCGGTTTAGAGGTCGATGTGGCAGAATATAAAG |
| NM_003769 | AAGCACCAGCTATGGCTACTCACGGTCTCGGTCTGGGTCAAGGGGCCGTG |
| NM_181314 | GCTGCCCTGCTCCACCCCACGTCTGGCCTTCTGTCCACTGTGCTGCAGGA |
| NM_003262 | CAGTGACAGGAGGGAAGATGATCGATCCCAGCACAGTAGTGGAAATGGAA |
| NM_006497 | TCGCTGGATGAAGCACGAGCCGGGCCTGGGTAGCTATGGCGACGAGCTGG |
| NM_007117 | GTCCCAGATCTTTCAATCTGACTGGCTCTCCAAACGTCAGCATCCAGGCA |
| NM_006759 | TATTAGAGAACAAGATAGTGTCTGGAAACCTTCGCATCTTGGACCACTGA |
| NM_014232 | CCCTGCACCCCCTCCAAACCTCACCAGTAACAGGAGACTGCAGCAGACCC |
| NM_006299 | AATCACCGAGGAATCCACAATATACAGAAACGGTACCACTGCAAGGAGTG |
| NM_003427 | CCTCACCATCCCCAGTCCTGATGCCGACCTGGCCACATCTGGCACACATA |
| NM_000072 | GAGACCTGTGTACATTTCACTTCCTCATTTTCTGTATGCAAGTCCTGATG |
| NM_002211 | TGGTTGCTGGAATTGTTCTTATTGGCCTTGCATTACTGCTGATATGGAAG |
| NM_004242 | CCGTTGCTTTACTTTTTGCTTCACCGACATAGTCATTATGCCGAAGAGAA |
| NM_005589 | CCAATGTTCTCATTCACCGGCTCTCGATCCTCCTTCAGGGGAGACACCAA |
| NM_022067 | TAGAACTAGACCTGGAAGTTTCCAGTCCCTTTCTGATGCTCTGTCAGACA |
| NM_001752 | TACCGTGCTCGAGTGGCCAACTACCAGCGTGATGGCCCGATGTGCATGCA |
| NM_001793 | CGGCAACTTTATAATTGAGAACCTGAAGGCGGCTAACACAGACCCCACAG |
| NM_003159 | GTAAACCAAGCTGCGCTCCTGACATACCATGAGAATGCGGCACTGACGGG |
| NM_156039 | CGACTGTGTCTTTGGGCCACTGCTCAACTTCCCCCTCCTGCAGGGGATCC |
| NM_004941 | ATGAGGAACCCAATGCCTGGAGAATATCTCGAGCTTTCCGACGGCGCTGA |
| NM_021071 | CCTGATCCTATAGCTATTGCATCTCTCTCCTTTTTGACCAGTGTCATCAT |
| NM_005665 | GCTTCAAGGACAGCTTAACAAGTCTGATTCTAACCAGTATATTGGGGAAC |
| NM_004475 | CTGTGCATGCCCTCACAGGCGTGGACCTGTCTAAGATACCCCTGATCAAG |
| NM_002133 | AGAGGGAAGCCCCCACTCAACACCCGCTCCCAGGCTCCGCTTCTCCGATG |
| NM_004125 | CGAGCGCCCTGCAGCGCTTGGTAGAGCAGCTCAAGTTGGAGGCTGGCGTG |
| NM_002102 | TGGTGGGCGATGGCTCGTGTTATTTTTGAGGTGATGCTTGTTGTTGTTGG |
| NM_005318 | CCTCGGGGTCCTTCCGGCTAGCCAAGAGCGACGAACCCAAGAAGTCAGTG |
| NM_002108 | GAATCAAGACCTCTTTCTCCAACAGCCTTTTCACTGCAATTTCTGCACAA |
| NM_006644 | ATTTTGGTGCTGAACCTCCACATCAGAATGGTGAATGTTACCCTAATGAG |
| NM_181430 | GCCTCAGAAGGCCCCCATGTGCCAGCTCAGACTGGAGTTGCCTCAGTTGA |
| NM_014780 | CCTCACCTTCCATACCCTACAGATTCGCTCCCGGGGTGTGCCCTATGCCT |
| NM_005123 | ACGACCACAAGTTTACCCCACTTCTCTGTGAAATCTGGGACGTGCAGTGA |
| NM_000917 | CATGACCCTGAGACTGGAAAATTGACCACAGCACAGTACAGAGTATCTAA |
| NM_000869 | GACAAGCTGCTATTCCACATTTACCTGCTAGCGGTGCTGGCCTACAGCAT |
| NM_006218 | GATGTGTTACAAGGCTTATCTAGCTATTCGACAGCATGCCAATCTCTTCA |
| NM_005084 | CCAGGGACCAACATTAACACAACCAATCAACACATCATGTTACAGAACTC |
| NM_002890 | GCTCTCACCAAACTGCCCACTTCGTTGCTTGCTGAGACTCTCGGGCCAGG |
| NM_134269 | TCTACCGCTGTCTGGTCCAGAAGGGGCTGGTAAAAACCAAAAAGTCCTAA |
| NM_004177 | AGATGAAACGAAAAAAGCTGTGAAATACCAGAGTCAGGCCCGGAAGAAAT |
| NM_003242 | CCTGTGTCGAAAGCATGAAGGACAACGTGTTGAGAGATCGAGGGCGACCA |
| NM_003451 | CACTAACCTTATAATGCACAAGCGAATCCACAATGGCCAGAAACTCCATG |
| NM_019841 | CTGGGGCACTTGAATCTTGGACTGAACCTTAGTGAGGGGGATGGAGAGGA |
| NM_003177 | TCAGCGGGTGGAATAATCTCAAGAATCAAATCATACTCCTTCCCAAAGCC |
| NM_000277 | CGCTACGACCCATACACCCAAAGGATTGAGGTCTTGGACAATACCCAGCA |
| NM_053056 | AACAAACAGATCATCCGCAAACACGCGCAGACCTTCGTTGCCCTCTGTGC |
| NM_000873 | TATGAGCCTGTGTCGGACAGCCAGATGGTCATCATAGTCACGGTGGTGTC |
| NM_001092 | CTTCAGCTACGGGACGGACGAGTACGACGGAGAGGGGAATGAGGAGCAGA |
| NM_004454 | GGAGGACACCCTGCCGCTGACCCACTTTGAAGACAGCCCCGCTTACCTCC |
| NM_005225 | TTTCAGATCTCCCTTAAGAGCAAACAAGGCCCGATCGATGTTTTCCTGTG |
| NM_001760 | ACCCGCCATCCATGATCGCCACGGGCAGCATTGGGGCTGCAGTGCAAGGC |
| NM_004064 | AATAAGGAAGCGACCTGCAACCGACGATTCTTCTACTCAAAACAAAAGAG |
| NM_000075 | CAGAGGATGACTGGCCTCGAGATGTATCCCTGCCCCGTGGAGCCTTTCCC |
| L27560 | CGCCAAGCCCCCCTCAAGGTGGGACAGTACCCCGGACCCATCCACTCACT |
| NM_002467 | AGGAGGAACGAGCTAAAACGGAGCTTTTTTGCCCTGCGTGACCAGATCCC |
| NM_001175 | ACCTCAGCTGGGAGTGGAACCTGTCGATTAAGAAGGAGTGGACAGAATGA |
| AB007921 | GGTGCACAGTGGACATTTGGGGAGCGTTGTGGGTGACCCCCACACAGGCA |
| NM_000724 | TGTAACGTCACCCCACTCCAAAGAGAAAAGAATGCCCTTCTTTAAGAAGA |
| NM_005187 | GAGGACATAGCCATGGCCCACCACTTCCGAGATGCCTACCGCCACCCAGA |
| NM_002987 | GGGGCTTCTCTGCAGCACATCCACGCAGCTCGAGGGACCAATGTGGGCCG |
| NM_001803 | AGCGGAGGCATTTTCCTTTTCTTCGTGGCCAATGCCATAATCCACCTCTT |
| NM_014376 | GAGCTGGCCAGATACATTGAGACCAGTGCTCACTATGAAGAGAACAAGTC |
| NM_182908 | ACGTCAACGGGGAGAACATTGCGGTGGCAGGCTACTCCACTCGGCTCTGA |
| NM_004434 | TACCCCTGCTCGCAGTTCAGGGCTCCAAGCCACATCTACGGCGGGCACAG |
| NM_014210 | CAAAACAGCTCACAGGACCCAACCTAGTGATGCAATCTACTGGAGTGCTC |
| NM_013451 | AATTGGACAAAATAAATTAATTGGCACGGCGACTGTAGCCCTGAAGGACC |
| NM_003088 | ATCGTGTTCCGCGGGGAGCATGGCTTCATCGGCTGCCGCAAGGTCACGGG |
| NM_000156 | ACGGCAGACACACAAGGTCATCCCCTTGAAAGGCCTGTGGGAGGATGTGG |
| NM_002048 | TACGATGAGGACTACGATGACGAGCAGCGCACCGGGGGCGCGGGTGGTGA |
| NM_002051 | GACGGTCAAGGCAACCACGTCCCGCCCTACTACGGAAACTCGGTCAGGGC |
| NM_000169 | CTTCCCTGGGTAAAGGAGTGGCCTGTAATCCTGCCTGCTTCATCACACAG |
| BM722299 | CCTGCTCTCGCACCGTCCGAGCGGAGCTTTCGTTTTCAGTGAGCCAGGTG |
| NM_002224 | GTGAAGAACAAGACCGACTACACGGGCCCTGAGAGCTACGTGGCCCAGAT |
| NM_005335 | GGGAAGTGATGAGCTTTCCTTTGATCCGGACGACGTAATCACTGACATTG |
| NM_002167 | CAGACAGCCGAGCTCGCTCCGGAACTTGTCATCTCCAACGACAAAAGGAG |
| NM_004633 | AATACCCTGAGTTTTCAGACACTACGCACCACAGTCAAGGAAGCCTCCTC |
| NM_015187 | GGTTTTTTGGACCCCTGAAGTACTATCGCCTTCGCAGTCTGCACCCCGCC |
| NM_015196 | AATACTACCCGGGGTTCAACCCGTTTCGCGCCTATATGAACCTGGACATA |
| NM_005556 | GTGGCATTGGGCTGACCCTCGGGGGAACCATGGGCAGCAATGCCCTGAGC |
| NM_002752 | CAGACAGCAGTCTTGATGCCTCGACGGGACCCCTTGAAGGCTGTCGATGA |
| NM_005933 | GTTGCCCCTTCCCCATTAGTACTGGGGCAAGAACGGAGTTGCTTCTATAA |
| NM_005098 | AACCACCGCTTAAATCGGACTGGAACTCACTTGATGGGATTATTCGTTAA |
| U50748 | GGTGTGCTTTTGACTGACAAGTCAAGGGTATCGTGCCCATTCCCAGCCCC |
| AB033114 | CTCCCAAATTCTCCTTATCCACCATTCACATACGACTGACGGCCAAAGGA |
| NM_002499 | AGACAGGGCCAATTCCACAGAATCCGTTCGAAATACCCCCAGCACTGACA |
| NM_004289 | GCCAGTCAATCCCAACCACTATGCTCTCCAGTGTACCCATGATGGAAGTA |
| NM_005654 | TTCGTGCTCAACGCGGCCCAGTGCTCTATGCCGCTGCACGTGGCGCCGTT |
| NM_020992 | AAGTGTTAAAGCTCCTGTCACTAAAGTGGCTGCGTCGATTGGAAATGCTC |
| NM_003311 | CTGCTGGAACGCGGCCATCGCGCTGGCGCTCATCGATTTCCAGAACCGCC |
| NM_002661 | AGAAGATATGTTCAGCGATCCCAACTTTCTTGCTCATGCCACTTACCCCA |
| NM_015568 | CAAGGCTCCCTTGATCGGAGGCAGAACTTCACCGTACAGCAGCAATGGGA |
| NM_006115 | CCAGCTTACAACCTTAAGCTTCTACGGGAATTCCATCTCCATATCTGCCT |
| NM_080588 | GCTAGACAGAGGGGGGATGATCCAGACGGCAGAGCAGTACCAGTTCCTGC |
| NM_021038 | GCTGCACAGAAATTAATGCGAACAGACAGACTTGAGGTATGTCGAGAGTA |
| NM_006498 | TGAGCGTAAGGGGCGGGTTCAACATGTCCTCTTTCAAGTTAAAAGAATAA |
| NM_014904 | GGGGATCTGATAGCCCTTGTGACTTGAAATCACCTAATGCATTTAGTGAA |
| NM_002870 | CTGGCCCGGGACATCTTGCTCAAGTCAGGAGGCCGGAGATCAGGAAACGG |
| NM_002972 | TGCGGCCCTACAGCAACGTGTCCAACCTGAAGGTGTGGGACTTCTACACT |
| NM_003569 | GAAAATGCAGAGGTGCACGTTCAGCAAGCAAATCAGCAGCTGTCAAGGGC |
| NM_015055 | AGCCCTCCACCACACAAAGAAGCCCGCCAGCGTCGGAAAGAACTCCGGAA |
| NM_015173 | CCATAGTTGACTTTATAAAAAGCACGCTACCCAACCTTGGCTTGGTACAG |
| NM_016021 | ACCCAACCTGTAGCTAAGAATACCTCCATGAGCCCTCGACAGCGCCGGGC |
| NM_005573 | GGAGAGGAGGTTGCTCAAAGAAGTACAGTCTTTAAAACAACCATACCTGA |
| NM_031407 | TTGAAGACTCCTATCGTGAGCTGCATCGCAAATCCCCCGAAGAAATGAAG |
| NM_004665 | AGGTGCTGAAAGATGGGCGTTTGGTAAACAAGAATGGATCATCTGGGCCT |
| NM_001783 | AACGAGTCATACCAGCAGTCCTGCGGCACCTACCTCCGCGTGCGCCAGCC |
| NM_000626 | GCTTCACGGTGAAAATGCACTGCTACATGAACAGCGCCTCCGGCAATGTG |
| NM_006762 | AAGTGCGTGTGGCGGTGCTACAGATTGATCAAGTGCATGAACTCGGTGGA |
| NM_002838 | CACACCACAGCTCTGCTGCCTTACCTGCACGCACCTCCAACACCACCATC |
| NM_173216 | ACCTGCTTGGAAAAGCCACACTGCCTGGCTTCCGGACCATTCACTGCTAA |
| NM_002163 | CGGATGTTTCCAGATATTTGTGCCTCACACCAGAGATCATTTTTCAGAGA |
| NM_002339 | GTTTGTGGCCACCGGGCATGGGAAGTATGAGAAGGTGCTTGTGGAAGGGG |
| NM_152866 | AAGAAACAGAGACGAACTTTCCAGAACCTCCCCAAGATCAGGAATCCTCA |
| NM_006495 | GGAGTTCTCTATTCCTCCCAACTCTGATCAAGATCTTAATGAATCCCTGC |
| NM_005246 | AGCAAGCAAGAGAGCAAGTAGAAAGAGGATACCGGATGTCAGCTCCCCAG |
| NM_000698 | ACGGTCACCGTGGCCACTGGCAGCCAGTGGTTCGCCGGCACTGACGACTA |
| NM_004271 | CAAAGAGCTTTTTCTTGACCTAGCTCTCATGTCTCAAGGCTCATCTGTTT |
| NM_001715 | TCCTCCTGATGGAAGTTGTCACTTATGGGCGGGTGCCATACCCAGGGATG |
| NM_003254 | GGGTTCCAAGCCTTAGGGGATGCCGCTGACATCCGGTTCGTCTACACCCC |
| AK090461 | TCCTGAGCCTCTTCTACAGTACCACCGTCACCTTGTTCAAGGTGAAATGA |
| NM_004071 | CCAGGAAACGTAAATATTTTCACCACGATCGATTAGACTGGGATGAACAC |
| NM_000757 | CCCCTGCCCCGTTTTAACTCCGTTCCTTTGACTGACACAGGCCATGAGAG |
| NM_020548 | GACATAAATACAGAACGGCCCGGGATGTTGGACTTCACGGGCAAGGCCAA |
| NM_002017 | AGCGACGACCAGTCCCTCTTTGACTCAGCGTACGGAGCGGCAGCCCATCT |
| NM_000206 | ATAGCCCCTACTGGGCCCCCCCATGTTACACCCTAAAGCCTGAAACCTGA |
| NM_004559 | CTTCAATTACCGACGCAGACGCCCAGAAAACCCTAAACCACAAGATGGCA |
| NM_021975 | CTCCCCAATGGCCTCCTTTCAGGAGATGAAGACTTCTCCTCCATTGCGGA |
| NM_138933 | CCCCTTCTGTTAGAGAAATTTACATGAATGTACCTGTAGGGGCTGCGGGA |
| NM_005919 | GGGGCCCCGGCGACTTTCCTAAGACCTTCCCCTATCCCTTGCTCCTCGCC |
| NM_000026 | ATCATTTACTGGATCCTTCTTCTTTCACTGGTCGTGCCTCCCAGCAGGTG |
| NM_000687 | TGGGCATGTCCTGTGATGGCCCCTTCAAGCCGGATCACTACCGCTACTGA |
| NM_001621 | CGTCAGAAGCCAGACCTTTTCCTGATTTGACATCCAGTGGATTCCTGTAA |
| NM_001647 | ATCAAATCGAAGGTGAAGCCACCCCAGTTAACCTCACAGAGCCTGCCAAG |
| NM_001664 | CAGAGATATGGCAAACAGGATTGGCGCTTTTGGGTACATGGAGTGTTCAG |
| NM_152862 | GGAAGCAGTAGAAGTAACATTTGCAGATTTCGATGGGGTCCTCTATCATA |
| NM_005171 | TCTGTTCCAACTCCCATCTATCAGACTAGCAGCGGACAGTACATTGCCAT |
| NM_001519 | GGAGAGGCTTTGCTCCCAAGCTCTCCCACCCTCGAAGCTGAGCCTGCCAG |
| NM_000900 | TCAATAGGGAAGCCTGTGATGACTACAGACTTTGCGAACGCTACGCCATG |
| NM_001746 | CCGTGGCTGTGGGTAGTCTATATTCTAACTGTAGCCCTTCCTGTGTTCCT |
| NM_004347 | GCGAAAGAATCGCGTGGCTCATCAAATGTTTACCCAAACACTTCTCAATA |
| NM_005201 | TCTTCAACTACCTAGGAAGACAAATGCCTAGGGAGAGCTGTGAAAAGTCA |
| NM_000560 | GCTATGCGAAAGCAAGACTGTGGTTTCATTCCAATTTCCTGTATATCGGA |
| NM_004356 | TGACCACCTCAGTGCTCAAGAACAATTTGTGTCCCTCGGGCAGCAACATC |
| NM_001769 | TCAGTATGATCTTGTGCTGTGCTATCCGCAGGAACCGCGAGATGGTCTAG |
| NM_001827 | CCGGCATGTTATGTTACCCAGAGAACTTTCCAAACAAGTACCTAAAACTC |
| NM_000397 | AACCCTGAGTAAACAAAGCATCTCCAACTCTGAGTCTGGCCCTCGGGGAG |
| NM_004728 | GGAAGGCAGTCGAGGCTTCAGGGGACAGCGGGACGGAAACAGAAGATTCA |
| NM_021907 | TTCTTCCTTTCCTCATATAAATGTTTCTCCTCTTCTCCCCGCACATCATC |
| NM_006169 | CTGGCTACACAATCGAATGGTTTGAGGTGATCTCGCAAAGTTATTCTTCC |
| NM_004416 | TACTGGGCCGGTCCATCCGGCCCTGGCAGGGATGACCGGGATACTGCTGT |
| NM_001948 | CTGCTCTGAAGAGACACCCGCCATTTCACCCAGTAAGCGGGCCCGGCCTG |
| NM_001951 | GAGTCGAGTTCATCTAAGCCCGTGGTTTTTCCTGTTCCCCCACCTGATGA |
| NM_001961 | TGACCACTGGCAGATCCTGCCCGGAGACCCCTTCGACAACAGCAGCCGCC |
| NM_003752 | CTACCGCAAAAACGAGGGCTACATGCGCCGCGGTGGCTACCGCCAGCAGC |
| NM_001436 | GGACACTTTGTGATTTCCATTAAGGCCAACTGCATTGACTCCACAGCCTC |
| NM_005252 | GACATGGACCTATCTGGGTCCTTCTATGCAGCAGACTGGGAGCCTCTGCA |
| NM_000146 | CTGGGCTGGGCGAGTATCTCTTCGAAAGGCTCACTCTCAAGCACGACTAA |
| NM_012197 | TACAGGACTTGGAACACCATTTAGGGCTTGCCCTCAATGAGGTGCAGGCA |
| NM_000435 | TCCCAGTGAGCACCCTTACCTGACCCCATCCCCCGAATCCCCTGAGCACT |
| NM_005890 | CAATATGGAGAACAGCTTTGACGATGTTTCTTGCCTCTCTCCCCAGAACC |
| NM_002065 | GAAGATCGTCGCCCCTCTGCCAACTGCGACCCCTTTTCGGTGACAGAAGC |
| NM_002080 | AATTGGCATGTTCTGTTTCACAGGGCTAAAGCCTGAACAGGTGGAGCGGC |
| NM_002092 | CAAGGATCGGTCCCACGTTCATCATAGGTATATTGAACTGTTCCTGAATT |
| NM_004493 | TGGCTCCCATAGGTATCCGGGTGATGACCATTGCCCCAGGTCTGTTTGGC |
| NM_004964 | AATGCTGCCGCACGCACCTGGGGTCCAAATGCAGGCGATTCCTGAGGACG |
| NM_004494 | AAAAGAATAGCACCCCCTCTGAGCCCGGCTCTGGCCGGGGGCCTCCCCAA |
| NM_002129 | GCTATGACAGGGAGATGAAAAATTACGTTCCTCCCAAAGGTGATAAGAAG |
| NM_031266 | TATGGCTATTACGGCTACGGCCCCGGCTACGACTACAGTCAGGGTAGTAC |
| NM_014413 | CTCAAGACAAAGGGGTGAGGGATGACGGAAAGGATGGGGGCGTGGGATGA |
| NM_198336 | GCTTTTCTAGCTACGCTGATCACGCAGTTTCTCGTGTATAATGGTGTCTA |
| NM_000212 | ATAAAGAGGCCACGTCTACCTTCACCAATATCACGTACCGGGGCACTTAA |
| XM_290793 | CATCCATCTGGCAGATTGGAAGCGGGGAAGGAATGAAGCGTGTCCTGACT |
| NM_005956 | GGACGGCCCAGTTTGATATCTCTGTGGCCAGTGAAATTATGGCTGTCCTG |
| NM_005962 | GAGAGTGTGAACATGGCTACGCCTCTTCATTCCCGTCCATGCCGAGCCCC |
| NM_000265 | CGCCTCAGCCAGGACGCCTATCGCCGCAACAGCGTCCGTTTTCTGCAGCA |
| NM_005601 | GCCAACAGGGCATGGGGACATCATATCAGGCTACATCCACGTGACGCAGA |
| NM_002512 | GAAGAACTGGTTGACTACAAGTCTTGTGCTCATGACTGGGTCTATGAATA |
| NM_000270 | TTGTCTCCATTCTTATGGCCAGCATTCCACTCCCTGACAAAGCCAGTTGA |
| NM_004152 | TAACTGGCGAACAGTGCTGAGTGGCGGCAGCCTCTACATCGAGATCCCGG |
| NM_001604 | TGCAGATGCAAAAGTCCAAGTGCTGGACAATCAAAACGTGTCCAACGGAT |
| NM_002541 | CCAACTTCGACATCAATCAGCTATATGACTGCAATTGGGTTGTTGTCAAC |
| NM_013232 | GAGGTTGACGGATATATTCAGACGTTACGACACGGATCAGGACGGCTGGA |
| NM_021129 | TCTGCCTGCACAGTACCAACAGACGTGGATAAGTGGTTCCATCACCAGAA |
| NM_001198 | AAGAGAAGTGTACATACATTGTGAACGACCACCCCTGGGATTCTGGTGCT |
| NM_006406 | TTGGGCCAATAAGGATTCCACTTCTTTCAGATTTGACCCATCAGATCTCA |
| NM_002836 | GTCAAGAGCCTGCGGCTACAGAGGCCACACATGGTCCAGACACTGGAACA |
| NM_002871 | GACCAATTGGCTGGCATTGCCTAGATGACAAGAACAGTTTCTATGTGGCC |
| NM_002881 | AATCACAGAACATGAATCCTTTACAGCAACTGCCGAATTCAGGGAACAGA |
| NM_005777 | AACCAGCAAAAGACAGTCCAACGAGACTTATCGAGATGCTGTTCGAAGAG |
| NM_004159 | CTTCTAGTCTTCTGGTTGAAGCTGCGCCTTTAGATGACACGACCCTACCC |
| NM_002923 | AGAACAACTCTTATCCTCGTTTCTTGGAGTCAGAATTCTACCAGGACTTG |
| NM_000976 | CTGGGGACTGCCCAGTCAGTGGGCTGTAATGTTGATGGCCGCCATCCTCA |
| NM_000983 | GAAGAAGAATAATCTACGTGACTGGTTGCGCGTAGTTGCTAACAGCAAAG |
| NM_000969 | CCTCACAGTACCAAACGATTCCCTGGTTATGATTCTGAAAGCAAGGAATT |
| NM_000970 | CTCCAGGGCTACCTGCGATCTGTGTTTGCTCTGACGAATGGAATTTATCC |
| NM_000661 | GTTATCCAGGAGAATGGGTCTCTTGTTGAAATCCGAAATTTCTTGGGTGA |
| NM_002951 | CATCCTTCACCCCTGTAGGGGATGTTTTTGAACTAAATTTCATGAACGTC |
| NM_001028 | ACAGAGCTCAAGTAATTTACACCAGAAATACCAAGGGTGGAGATGCTCCA |
| NM_001031 | TGGAATTCATGGACGACACGAGCCGATCCATCATCCGCAATGTAAAAGGC |
| NM_001007 | CCTGTGTATGGTGACTGGAGGTGCTAACCTAGGAAGAATTGGTGTGATCA |
| NM_001009 | ACTCCACACGCATTGGGCGCGCCGGGACTGTGAGACGACAGGCTGTGGAT |
| NM_001011 | ATTGTGGGCAAGAGAATCCGCGTCAAACTAGATGGCAGCCGGCTCATAAA |
| NM_001013 | TGCTGCGGCGGCTGGTCCGCATTGGGGTGCTGGATGAGGGCAAGATGAAG |
| NM_003011 | TCGGCGCAGGCGGCCAAAGTCAGTAAAAAGGAGCTCAACTCCAACCACGA |
| NM_006427 | TGGACCACGTGTGGGATGAAGGCTGTGCCGTCGTTCACCTGCCAGAGTCC |
| NM_001636 | TTCTTCAAGGGTGCGTGGTCCAACGTCCTGCGGGGCATGGGGGGCGCCTT |
| NM_006516 | GCTTCCGGCAGGGGGGAGCCAGCCAAAGTGATAAGACACCCGAGGAGCTG |
| NM_002860 | ATGCCAGCACTCGCTTTTCTGATGGTTACCGCTTTGGACTGGGAGCTGAA |
| NM_003134 | ATTCCAAAGAAAGGTACTGTGGAGGGCTTTGAGCCCGCAGACAACAAGTG |
| NM_006280 | ATCGGCCTTGTGATCTACTACTTGGCCTTCAGTGCGAAGAGCCACATCCA |
| NM_003153 | CAATCTGGGATCTCAATGTCCCACATGGACCTAAGGGCCAACCCCAGTTG |
| NM_005801 | ACAGGGTGACCAACGCAAGAACATATGCCAGTTCCTCGTAGAGATTGGAC |
| NM_003258 | ATTGGGGGAGCAGACAAGTACCACTCCGTGTGTCGGCTCTGCTACTTCAA |
| NM_014220 | GGAATGGAATGTATCTCTGTTTTCTATCCTCTTGGCTCTTGGTGGAATTG |
| NM_003318 | TAAAGAATGACTTTCCACCTGCTTGTCAGTTGTCAACACCTTATGGCCAA |
| NM_006000 | CACCACCTTCTTCTGTGAAACTGGTGCTGGAAAACACGTACCCCGGGCAG |
| NM_006009 | CCTCGATATTGAGCGTCCAACCTATACTAACCTGAATAGGTTAATAGGTC |
| NM_006002 | ATGGAGCGCGACCCTGATGAACTAAGATTTAATGCGATTGCTCTTTCTGC |
| NM_000375 | ATCAAATTAAGTTTGCAGCCATCGGCCCCACTACGGCTCGCGCGCTGGCC |
| NM_003380 | GGACATTCCTGATTAAGACGGTTGAAACTAGAGATGGACAGGTTATCAAC |
| NM_000377 | CCCCTATTGTGGGGGGTAACAAGGGTCGTTCTGGTCCACTGCCCCCTGTA |
| NM_005080 | TGTCCTCTCTGCTTGGTGTAAACCATTCTTGGGAGGACACTTTTGCCAAT |
| NM_153380 | TCCCCCATAGCCCTTCCTCTACTAAGAATGAGAATGCTAAAACAGGAGCA |
| NM_012203 | CATCAGCAGGGGCGACGTCGTAAACCAGGACGACCTGTACCAGGCCTTGG |
| NM_001785 | CGTGTACATGACCAAGCCGGATGGTACGTATATTGTCATGACGGTCCAGG |
| NM_002341 | GGGGCGAGAGGGTGTACGTCAACATCAGTCACCCCGATATGGTGGACTTC |
| NM_001152 | CCGGGTTGACTTCCTATCCATTTGACACCGTTCGCCGCCGCATGATGATG |
| NM_144488 | GGGCCCTACAGAGATGCTCCGAGGCATGTACCTCACTCGCAACGGGAACC |
| NM_021966 | GAAGCAGCACGCCTGGCTGCCCTTAACCATCGAGATAAAGGATAGGTTAC |
| NM_002350 | TTATGTGAGAGATCCAACGTCCAATAAACAGCAAAGGCCAGTTCCAGAAT |
| NM_001774 | TGTGCAGAAACCTGGACCACGTCTACAACCGGCTCGCTCGATACCGTTAG |
| NM_013230 | CAGCCAGTCTCTTCGTGGTCTCACTCTCTCTTCTGCATCTCTACTCTTAA |
| NM_006325 | GCACAGTATGAGCACGACTTAGAGGTTGCTCAGACAACTGCTCTCCCGGA |
| NM_005340 | AATGTGCTGCTGATCTGGGCCTGAATAAGGGTTATCGAATGGTGGTGAAT |
| NM_002046 | ACAGCAACAGGGTGGTGGACCTCATGGCCCACATGGCCTCCAAGGAGTAA |
| NM_000334 | TGCTTGCCAACACCATGAGCAAGATGTATGGCCACGAGAATGGGAACAGC |
| NM_000484 | GTGGTTCGAGAGAAGTGGTATAAGGAAGTACATTCTGGCCAGGCACGATG |
| NM_170662 | GATAGCCCAGAATAATGTCGAAGTTGCCCGGAGCATCCTCCGAGAATTTG |
| NM_006904 | CCATGACCTCCAGGTTAGGATTAATTGAGTGGCTTGAAAATACTGTTACC |
| NM_133480 | GCTAATTGCCCAGGGCCTTTTGGAGTCTGAGGACCGCCCCGCAGAGGACT |
| NM_000633 | CCCTGGTGGGAGCTTGCATCACCCTGGGTGCCTATCTGGGCCACAAGTGA |
| NM_002086 | TTTCTTATCCGAGAGAGTGAGAGCGCTCCTGGGGACTTCTCCCTCTCTGT |
| NM_005248 | CAGCATCCCTGTACGAGGCCATGGAACAGACCTGGCGTCTGGACCCGGAG |
| NM_002447 | AACAGCCGCAGTTCTCACCCATGCCAGGGAATGTACGCCGGCCCCGGCCA |
| NM_003656 | AGATACAGCTCTAGATAAGAATATCCACCAGTCGGTGAGTGAGCAGATCA |
| NM_138980 | AAGACTAAAAATGGTGTAGTAAAAGGACAGCCTTCTCCTTCAGCACAGGT |
| NM_016072 | TAGAAGAGTGCCAGTCCTTGGATCCCTCCTAAATTTACCTGGAATTAGAT |
| NM_000785 | CACGGTGTCCAACACGCTCTCTTGGGCTCTGTATGAGCTCTCCCGGCACC |
| AF200348 | GTTCAAGCGCCTGCGAGATGGGGACAGGTTGTGGTATGAGAACCCTGGGG |
| BC060842 | GGGACATGTGAATTATATCCATGGAAGCAGTCAAGCCCAGTTTGTAGCTG |
| NM_006302 | TTTGGGAGCAGTACAGTGACCGCGATGGGCGAGGCATGTGCCGCCCTTTC |
| NM_020199 | GGAATACCATCGCCTAGATCAGAATGTTAATGAGGCAATGCCTTCTTTGA |
| NM_024006 | CTGCATTGTTTGTATCACCACCTATGCTATCAACGTGAGCCTGATGTGGC |
| NM_002229 | AAGGTGAAGACGCTCAAGGCCGAGAACGCGGGGCTGTCGAGTACCGCCGG |
| NM_024099 | CCCCTTCAAAGCCAGCCAGGACTAGTGAACCACAGCTCAAAAGGCAAAAG |
| NM_006378 | CGGATAGGGAGGACTCACAGAGGATCGACGACCTTTCTGCCAGGGACAAG |
| NM_033334 | CTGCCCCTCCTCTTTAAGGTGGTGCTGCATTCCTGCAAGACCAGTGTGGG |
| NM_002814 | ACACTGAGGGTAACACTCCTCTACACTTAGCCTGTGATGAGGAGAGAGTG |
| NM_017771 | CCACAAACAGTACTATTTTGCAGATGCTCATGTAAGCAGCTTTTCGAGAG |
| NM_003002 | TGGGGCCTTGGACAAGTTGTTACTGACTATGTTCATGGGGATGCCTTGCA |
| NM_003627 | GTGTTCGCCTTGCTTCAGCAGCCACTTTTCATGGCGATGGTGGGACCCCT |
| NM_005638 | TGTCACCTTCAAAACTACCAGCAGAAATCTTGCTCGAGCCATGTGTATGA |
| NM_003288 | CAGACCTCTACAAGAAGACTCAGGAAACTCTTTCACAGGCAGGACAGAAG |
| NM_003302 | TTCTTTGGTGTTCACTGGCATTTTGGGGGTGCACTGGCACTTTGGAACAA |
| NM_016308 | GCTTCGTGATGAAAGGAAGAACCCAGATTCACAGTATGGTGAACTTATTG |
| NM_006113 | CATTTCTCACGGCCTGTTGTGAGACGTTTGGAATGAGGAAAAGTGAACTT |
| NM_006802 | CATGGCATGAGGTGTTTGGGCATCCCAAATACTGCTCACTTTGCTAATGT |
| NM_004184 | AAACCAAGGTCAATAAGCATGCGTTTTCTGGAGGGAGAGACACCATCGAG |
| NM_000089 | AAGAGGGGAACGCGGTCCCCCAGGTGAGAGTGGTGCTGCCGGTCCTACTG |
| NM_004951 | ACGGCAAGTCAGTGTATCGATTTCTAGTGCTGTGAAGTCAGCCCCTGAAG |
| NM_002600 | GAAAGTTACTGGTGAATAAAAGCATTCGGCAGCGTCGTCGCTTCACTGTG |
| NM_005178 | GAGTGCCAAGAAACCGTGCAGCTCTTGCTAGAGCGCGGTGCCGACATCGA |
| NM_001753 | ATTCAGTGCATCAGCCGTGTCTATTCCATCTACGTCCACACCGTCTGTGA |
| NM_002414 | GGAATGCCAACGCAGAGCCAGCTGTTCAGCGTACTCTTTTAGAGAAATAG |
| NM_001788 | AACAAGAATAAAGGGCAGCTGACTAAGAGCCCTCTGGCACAAATGGAAGA |
| NM_004067 | GAGTCTCTGGGTTCACTGAACACATTGAAGATGTCAAAATGGCATTTGAC |
| NM_016184 | GCTGCGTTGTGCTAAATTTTCGTAAATCACCCAAAAGATGGGGCTGGAAT |
| NM_005627 | TCCCCAACTCCATTGGCAAGTCCCCTGACAGCGTCCTCGTCACAGCCAGC |
| NM_006329 | AGGGCAGAGAATTTTACATGCGGCAAACGGGCCCCATCAGTGCCACCCTG |
| NM_016195 | GAGGAGGACTTGGTGAAATGTGAAAATAAGAAGAATGCTACACCCAGAAC |
| NM_006310 | CTGGGAAGAACTTTATAACCGATACCAGGGAGGATTCTTAATATCCAGAC |
| NM_003707 | AACAGATCATTAAAATCCGTGCCCAGACGGAAGGAATCAACATCAGTGAG |
| NM_001845 | ACGCTTACAGCTTTTGGCTCGCCACCATAGAGAGGAGCGAGATGTTCAAG |
| NM_006803 | AACATAACATCAGTTTCCGGGACAGTAGTTCCCTTGGACGCTTTGAAATA |
| NM_001219 | TGGGAGGAGTATAAAAATGCCACCTACGGCTACGTTTTAGATGATCCAGA |
| NM_001260 | GCGTTCCAATCCACATGCTGCCTATCCCAACCCTGGACCAAGCACATCAC |
| NM_001916 | CTGGGCATCTGAGCCAGAGCACGACCATCGAAAACGCATGGGGCTCAAGA |
| NM_000121 | GGCTCCCGCCGCCTGAGACACCCATGACGTCTCACATCCGCTACGAGGTG |
| NM_002394 | AGCGCTCCCTACTGCATGGGGACTTCCACGCGTTCTCCGCTGGGCCTGGA |
| NM_004483 | GGAAAGTGTGAAAGCTGCTAGTGAACTCTATTCTCCTTTATCAGGAGAAG |
| NM_000521 | CTGACAAGGCACCGCTGCAGGATGGTCGAACGTGGAATAGCTGCACAACC |
| XM_167711 | GAAAGCGCAGTCCTGAAAGTCAGGTCACGATTATGGGCCCACACCTTCCT |
| AL833311 | ACTCATATTCTCGGTGTCCTGCTATCACACAGAACAACTTTCTAACCTAG |
| NM_013995 | CTAGTGTTGCTGGCTTATTTTATTGGTCTCAAGCACCATCATGCTGGATA |
| NM_024321 | CCTTCGTCCCCCACGTGCTACAGAGAGCAGATTCCGCTCTCTCCTCTGCA |
| NM_000946 | CTAAAACAGGTCGCATCTCTGTGCCTATTGATTTGCAGAAAGTGGACCAG |
| NM_004176 | GGGGACTGGTCCGTGCTCAGTACCCCATGGGAGAGCCTGTACAGCTTGGC |
| NM_002958 | AGCAGCTGGTACAGTGCCTAACAGAGTTTCATGCAGCCCTGGGGGCCTAC |
| NM_005063 | AAGGCCGCCATCTTGGCCAGGATTAAAAGAACCGGAGATGGAAACTACAA |
| NM_003132 | AGTCTCTTCAAGGAGTCCTATTACCAGCTCATGAAGACAGCCCTCAAGGA |
| NM_005100 | AATGGCCAGAAAGGAGCCCTGAACGGTCAAGGAGCCCTAAACAGCCAGGA |
| NM_021822 | AACCTTGGGTCAGAGGACGGCATGAGACTTACCTGTGTTATGAGGTGGAG |
| NM_020993 | GGGGGATCTGGAAGGAGTGCCACCCTCTAAAAAGATGAAACTGGAGGCCT |
| NM_015099 | CAGGCAGCTGCTCGGGGTCCCCCACCACAGTCAGTAGCAGGTGGGAGAAG |
| NM_005127 | ACCTCAGCGATGATGGTGCAGCAACAGCTAGATGTTACACCGAAAGAAAA |
| NM_004010 | AGACATTTAATTCTCGTTGGAGGGAACTACATGAAGAGGCTGTAAGGAGG |
| NM_002305 | GCGGGAGGCTGTCTTTCCCTTCCAGCCTGGAAGTGTTGCAGAGGTGTGCA |
| NM_000237 | GACTGAGAGTGAAACCCATACCAATCAGGCCTTTGAGATTTCTCTGTATG |
| NM_006187 | ACAGAGCTACAACGGGACTTCATCATCTCTCGCCCTACCAAGCTGAAGAG |
| NM_005419 | TTGCCCTGTGATCTGAGACATTTGAACACTGAGCCAATGGAAATCTTCAG |
| NM_002390 | ACCCACGGGGGAGACGGAGAGATATAAAGGTCCCAGCGGCACCAACATCA |
| NM_016187 | TTGTACTTCCCCCACCTTAATGACATCTCAGGTTGCTTCAGAGCCTGGAG |
| NM_014207 | CATCGCAACCACACGGCAACCGTCCGATCCCATGCTGAGAACCCCACAGC |
| NM_005214 | TGCAGCAGTTAGTTCGGGGTTGTTTTTTTATAGCTTTCTCCTCACAGCTG |
| NM_003564 | GCTGATGAATCTGGGTGGGCTGGCAGTAGCCCGAGATGATGGGCTCTTCT |
| NM_002002 | AGCCAGCGAAGGTTCCGCGGAGTCCATGGGACCTGATTCAAGACCAGACC |
| NM_004001 | CCAAGGCCCCAGACTAAGGACGGCAGCGAAGCAGAGCTCCCTCGTTGGTG |
| NM_058176 | GCCTGTGGCCAAAGAGTTTGATCCAGACATGGTCTTAGTATCTGCTGGAT |
| NM_001558 | ATCTGACTGGAGCTTTGCCCATGACCTTGCCCCTCTAGGCTGTGTGGCAG |
| NM_006850 | TCCGGAGAGCATTCAAACAGTTGGACGTAGAAGCAGCTCTGACCAAAGCC |
| XM_042066 | CTCCATCAAGAGAGCTACTGAAGCATCCAGTCTTTCGTACTACATGGTAG |
| NM_033004 | CCAGCCAGCCGTGGAACCTCAGGTGCAACAGAGACGCCAGGAGATACTAG |
| NM_002835 | CCAACAGAAGCCACAGATATTGGTTTTGGTAATCGATGTGGAAAACCCAA |
| NM_000593 | GCAGCTCCTGTACGAAAGCCCTGAGCGGTACTCCCGCTCAGTGCTTCTCA |
| NM_002927 | CTGGATGGCATGTGAAACCTATAAGAAAATTGCCTCACGGTGGAGCAGAA |
| NM_005012 | GCATCTTTACTAGGAGACGCCAATATTCATGGACACACCGAATCTATGAT |
| NM_000655 | CATGGTTACTGCATTCTCTGGGTTGGCATTTATCATTTGGCTGGCAAGGA |
| NM_005449 | TCCAGATGCCTGCATATGCCAGTTCTTCCAAATTCGTAACCAGAGTTACC |
| NM_133378 | TAATATCCTCTGGGGTACAGTCATGTATAACAAGTTTTCTGACCCTGCCA |
| NM_032663 | AAAGAAGCGTAGAAAAGGGCTTGTGCCTGGCCTTGTTAATTTAGGGAACA |
| NM_030753 | TGTTAGTGTCCAGGGAGTTCGCGGATGCGCGCGAGAACAGGCCGGACGCG |
| NM_017935 | CAAGACAGAGCTCGGATAGAGAGTCCAGCCTTTTCTACTCTCAGGGGCTG |
| NM_003202 | AGACAGGTGGCCTAGCAGGCACAGGACACCTGGCCGCCTCCAGGAGCCTA |
| NM_030764 | TGATGCCGGCAAATATTACTGTAGAGCTGACAACGGCCATGTGCCTATCC |
| NM_023109 | GTGGGCCAGTCTACTGGGAAGGAGACCACTGTCTCGGGGGCTCAAGTTCC |
| NM_002738 | GATTTTTCACCCGCCATCCACCAGTCCTAACACCTCCTGACCAGGAAGTC |
| NM_006472 | CCTGAGTTCAAGTTCATGCCACCACCGACTTATACTGAGGTGGATCCCTG |
| NM_000417 | TTATTATCAGTGCGTCCAGGGATACAGGGCTCTACACAGAGGTCCTGCTG |
| NM_001775 | GCCAGGATCCCACCATAAAAGAGCTGGAATCGATTATAAGCAAAAGGAAT |
| NM_005574 | TGTGCGAACAGGACATCTACGAGTGGACTAAGATCAATGGGATGATATAG |
| XM_034274 | CTCTAGAACTTATTGAATCTGATCCTGTAGCATGGAGTGACGTTACCAGT |
| NM_001706 | CTTCCGCTACAAGGGCAACCTCGCCAGCCACAAGACCGTCCATACCGGTG |
| NM_000902 | CAGAAATGCTTTCCGCAAGGCCCTTTATGGTACAACCTCAGAAACAGCAA |
| NM_001102 | ACACCGGCCCCGACTCCGTGCCAGGTGCTCTGGACTACATGTCCTTCTCC |
| NM_001718 | CCCGAGTATGTCCCCAAACCGTGCTGTGCGCCAACTAAGCTAAATGCCAT |
| NM_033554 | GGACCAGCCGCTCCTCAAGCACTGGGAGGCCCAAGAGCCAATCCAGATGC |
| NM_002122 | AGGAGACTGTCTGGTGTTTGCCTGTTCTCAGACAATTTAGATTTGACCCG |
| NM_019111 | TAACTGTGCTCACGAACAGCCCTGTGGAACTGAGAGAGCCCAACGTCCTC |
| NM_002124 | GGAGCACCCAAGCGTAACGAGCGCTCTCACAGTGGAATGGAGAGCACGGT |
| NM_014745 | CGTCCTCCCTATCAGACGACCAGGTACCCGAGGCTTTCCTGGTCATGCTG |
| NM_006993 | TCCCGGCCCCGGTCACTATGGACAGTTTTTTCTTCGGCTGTGAGCTCTCC |
| NM_014366 | GAGCATAAGAGCCATCAAGGGCCCTCATTTGGCCAATAGCATCCTTTTCC |
| NM_003255 | AGTGCAAGATCACGCGCTGCCCCATGATCCCGTGCTACATCTCCTCCCCG |
| NM_002658 | ACAAGGACTACAGCGCTGACACGCTTGCTCACCACAACGACATTGCCTTG |
| NM_002704 | CTTGTATGCTGAACTCCGCTGCATGTGTATAAAGACAACCTCTGGAATTC |
| NM_000090 | TTCTCCCCAGTATGATTCATATGATGTCAAGTCTGGAGTAGCAGTAGGAG |
| NM_002026 | CTGAGAGATGGACAGGAAAGAGATGCGCCAATTGTAAACAAAGTGGTGAC |
| NM_001766 | GCTTTACCTCCCGGTTTAAGAGGCAAACTTCCTATCAGGGCGTCCTGTGA |
| NM_006614 | TGGTGCCTACGCTGGATCTAAGGAGAAGGGATCTGTTGAAAGCAATGGAA |
| NM_004445 | TACTGGGACATGAGTGAGCAGGAGGTACTAAATGCAATAGAGCAGGAGTT |
| NM_006058 | CCAGGCCTACAGAACCAGAGCCAGCTGATCTCAGATTGCCAAGAAACTAG |
| NM_052938 | GGGACACATATGGAGGACAAGGTTTCCTTAGACATCTATTCCAGGCTGAG |
| NM_004126 | GGGAATTCCAGAAGACAAGAACCCCTTTAAAGAAAAAGGCAGCTGTGTTA |
| NM_007360 | TGAGAGCCAGGCTTCTTGTATGTCTCAAAATGCCAGCCTTCTGAAAGTAT |
| NM_004225 | GAAGATCAAAGACAACCCACTGATCCAGCCCCCCTACGAGGTCTGCATGA |
| NM_005940 | AAGGCTTCCCCCGTCTCGTGGGTCCTGACTTCTTTGGCTGTGCCGAGCCT |
| NM_003800 | TGCCAAAGAAGTGAGCCATGAAATGGATGGACTTATTTTTCAGCCTACTG |
| NM_030956 | TGCTAGGTCAATGCACACAAACATGGCACAGGGTTAGGAAAACAACCCAA |
| NM_003453 | GGACCGAAACACCTTGGAAAATATGCTTGTACGGGTTCTTCTAGTAAAAG |
| NM_018136 | CCTGTAAGGACCAGAATAGTTTCAAGACTTAAGCCAGATTGGGTTTTGAG |
| NM_000682 | GCATCTACCTGATCGCCAAACGCAGCAACCGCAGAGGTCCCAGGGCCAAG |
| NM_181802 | GTCAGGACCATTCTGCTCTCCATCCAGAGCCTTCTAGGAGAACCCAACAT |
| XM_058619 | ACATTGCCCGTCAGAAGCTTTTAGGTTTGTTCTGTCAGTCAAAACCAAGG |
| NM_016343 | CCAAAGCTGGACTGGAGTCCAAGGGCAGTGAGAACTGTAAGGTCCAGTGA |
| NM_018248 | GCAAGCGTTCCACCATGAAAACAGTATTGAAGATTGGACCTAACAATGGA |
| NM_000194 | GAGCTATTGTAATGACCAGTCAACAGGGGACATAAAAGTAATTGGTGGAG |
| NM_004526 | TGGCAAGCACAAGGTACGTGGTGATATCAACGTGCTCTTGTGCGGAGACC |
| NM_002426 | CCAAGAACTTCCAAGGAATCGGGCCTAAAATTGATGCAGTCTTCTATTCT |
| NM_002608 | CTGTCTCTCTGCTGCTACCTGCGTCTGGTCAGCGCCGAGGGGGACCCCAT |
| AF108138 | GGTGCCCGAGGGGTGGTAGTTGGGTTCGAGGCAGAAGGGAGAGGGCTACC |
| NM_002692 | CTACGACAAATACCGAATGCCTCTGCATAAACCCTGGCTCTTTTCCAAGA |
| NM_003349 | TTCAAGCGTCTTACCTGAAGTCACAAAGCAAACTGAGTGATGAAGGAAGA |
| NM_001067 | CTCCTCGGGCAAAATGTGTACGGGCAAAGAAACCTATAAAGTACCTGGAA |
| NM_013282 | TGCAAGCACTGCAAGGACGACGTGAACAGACTCTGCCGGGTCTGCGCCTG |
| NM_002356 | GGCGGCTGTGGCCTCGTCGCCTTCCAAAGCGAACGGACAGGAGAATGGCC |
| NM_002592 | AACGGTGACACTCAGTATGTCTGCAGATGTACCCCTTGTTGTAGAGTATA |
| NM_003113 | ACTCCCAGGGAATTTGAAATTGAAGGAGACCGCGGAGCATCCAAGAACTG |
| NM_005292 | GCGCAGAAAAAGTTTCCGATCTGGTAGTCTACGGTCACTAAGCAATATAA |
| NM_000022 | CTCCCAGCTAACACAGCAGAGGGGCTGCTGAACGTCATTGGCATGGACAA |
| NM_005163 | TACAAGGAGCGGCCGCAGGATGTGGACCAACGTGAGGCTCCCCTCAACAA |
| NM_001295 | TGTTTCAGGCTCTGAAACTGAACCTCTTTGGGCTGGTATTGCCTTTGTTG |
| NM_005191 | AGAGAAGGAGGAATGAGAGATTGAGAAGGGAAAGTGTACGCCCTGTATAA |
| NM_006889 | CCTGAAAGATCTGATGAAGCCCAGCGTGTTTTTAAAAGTTCGAAGACATC |
| NM_001846 | TGGTGATGTCTGCTACTATGCCAGCCGGAACGACAAGTCCTACTGGCTCT |
| NM_004460 | ACCAGAACCACGGCTTATCCGGCCTGTCCACGAACCACTTATACACCCAC |
| NM_032682 | CCATCCAGAATGGGTCGGGCGGCAGCAACCACTTACTAGAGTGCGGCGGT |
| NM_006120 | CTTCTGTGGCTGCTACCCCACTCCTGGGCCGTCCCTGAAGCTCCTACTCC |
| NM_002120 | ATATGTGAGGACGCAGATGTCTGGTAATGAGGTCTCAAGAGCTGTTCTGC |
| NM_006010 | TACATCCGGAAGATAAATGAACTGATGCCTAAATATGCCCCCAAGGCAGC |
| NM_005533 | TACCCCAAGGACAGCAGGGCCTAGCAGTCTTCACCTCTGAGTCAGGCTAG |
| NM_001560 | GTTACTCATTGTTCCAGTCATCGTCGCAGGTGCAATCATAGTACTCCTGC |
| NM_000632 | TCCTGATCGTGAGCACAGCTGAGATCTTGTTTAACGATTCCGTGTTCACC |
| NM_004972 | GGGATCTAGCTCTTCGAGTGGATCAAATAAGGGATAACATGGCTGGATGA |
| NM_004995 | CTCCCAGAGGGTCATTCATGGGCAGCGATGAAGTCTTCACTTACTTCTAC |
| NM_172390 | ACCTTTCTCCCGCTGCCTACACCAAGGGCGTTGCCAGCCCGGGCCACTGT |
| NM_005384 | CAGAGGTTGTCTCACTCAAGAGACTTATAGCCACACAACCAATCTCTGCT |
| NM_001675 | ATTTGATAGAAGAGGTCCGCAAGGCAAGGGGGAAGAAAAGGGTCCCCTAG |
| NM_000960 | GCCGTGGGAACGTCGTCCAAAGCAGAAGCCAGCGTCGCCTGCTCCCTCTG |
| NM_000963 | CGGACTAGATGATATCAATCCCACAGTACTACTAAAAGAACGTTCGACTG |
| NM_003037 | AAACAAATTCCATCACAGTCTATGCTAGTGTGACACTTCCAGAGAGCTGA |
| NM_003121 | GCGCGCGCCCTCCGAAACTACGCCAAGACCGGCGAGATCCGCAAGGTCAA |
| NM_007315 | CTCGGATAGTGGGCTCTGTAGAATTCGACAGTATGATGAACACAGTATAG |
| NM_006290 | GGCAATGCCAAGTGCAACGGCTACTGCAACGAATGCTTTCAGTTCAAGCA |
| NM_007115 | CTGGCACATTAGACTCAAGTATGGTCAGCGTATTCACCTGAGTTTTTTAG |
| NM_012452 | AGTCCGGCCAAGTCTTCCCAGGATCACGCGATGGAAGCCGGCAGCCCTGT |
| NM_003820 | AAGGTGATCGTCTCCGTCCAGCGGAAAAGACAGGAGGCAGAAGGTGAGGC |
| NM_005104 | TCCTCTGCACAGCAAGTAGCAGTGTCACGCCTTAGCGCTTCCAGCTCCAG |
| NM_000043 | GCTGGAGTCATGACACTAAGTCAAGTTAAAGGCTTTGTTCGAAAGAATGG |
| NM_003326 | GAATGGCGGAGAACTGATTCTTATCCATCAAAATCCTGGTGAATTCTGTG |
| NM_005658 | GCCTACGTGAAGGACGACACAATGTTCCTCAAGTGCATTGTGGAGACCAG |
| NM_006060 | CGCTGCCACAACTACTTGGAAAGCATGGGCCTTCCGGGCACACTGTACCC |
| NM_002371 | AGGGTGTCATCCAGGTACGTTCCAGCCGGCCAGCCATGCTCCTCCAGTGA |
| NM_005608 | AGAGCAGGTCCCCGTGCGGGCTGAGGAAGCCAGAGACAGTGACACGGAGG |
| NM_005348 | TGAACCTATGGGTCGTGGAACAAAAGTTATCCTACACCTGAAAGAAGACC |
| NM_000033 | CTCGCGTGTGGTGGCCAACTCGGAGGAGATCGCCTTCTATGGGGGCCATG |
| NM_004915 | GAAGAGGTAAAGCAGACAAAACGATTAAAGGGGTTGAGAAAGGACTCCTC |
| NM_005186 | GATCAGCGTGAAGGAGTTGCGGACAATCCTCAATAGGATCATCAGCAAAC |
| NM_005166 | ATGCGTCTGTTCCAAGGGGTTTCCCTTTCCACTCATCGGAGATTCAGAGG |
| NM_015193 | GACACGCAGATCTTCGAGGACCCTCGAGAGTTCCTGAGCCACCTAGAGGA |
| NM_001178 | GCAACCGCAAACGGAAAGGCAGCTCCACTGACTACCAAGAAAGCATGGAC |
| NM_004317 | GTGAAGCTGCCGCTGTTACCCCATGAGGTGCGGGGGGCAGACAAGGTCAA |
| NM_001187 | GCTGGAGGTTGGAGCCTGAAGACGGCACAGCTCTGTGCTTCATCTTCTGA |
| NM_172171 | GGATCACCAGAAACTAGAACGTGAGGCTCGGATATGTCGACTTCTGAAAC |
| NM_016280 | GGCTGAGGTAGGTCTCCGGCTGGTACGTCTCTGGCTGGACACCCACACCT |
| NM_000743 | GGACAGCAGGATTGTTTCTGCAACCCCTGATGGCCAGGGAAGATGCATAA |
| NM_001762 | TAAAATTCAAGCAGAACATTCAGAATCAGGTCAGCTTGTGGGTGTGGACC |
| NM_001910 | CTGCAAAACGCCATTGGGGCAGCCCCCGTGGATGGAGAATATGCTGTGGA |
| NM_000778 | ACCTGCTTGTCTACCTGTCTCCTACCCACCTGTATCTCTTGTTGGGAGAA |
| U53532 | ATGAATATCTTGGTAACTCACCTCGACTTGTCATTACGCCTCTAACTGAC |
| NM_001939 | CACACACTCCCGAATTGAGCATTTTGCGAGCAGGCTTGCTGAGATGGAAA |
| NM_004091 | GGTAGGCAGGGGAATGTTTGAAGACCCCACCAGACCTGGGAAGCAGCAAC |
| NM_001441 | AGCTGCAGGGTGCCGTGCCCTTCGTGCACACCAATGTTCCACAGTCCATG |
| NM_000148 | TTTGCTGGCGATGGACAGGAGGCTACACCGTGGAAAGACTTTGCCCTGCT |
| NM_001777 | CTATACAACCTCCTAGGAAAGCTGTAGAGGAACCCCTTAATGCATTCAAA |
| NM_002049 | ATGCGGAAGGATGGTATTCAGACTCGAAACCGCAAGGCATCTGGAAAAGG |
| NM_002068 | AAGAAGGGCGCACGATCCCGACGCCTTTTCAGCCACTACACATGTGCCAC |
| NM_005275 | CCCGGGCCATACCCGATACTTTCAGACCTACTTTCTTACCCCCTCTGTGA |
| NM_002076 | CCGTCTCATGTTCAGCAATCGCGGCAGTGTCAGGACTCGAAGATTTTCCA |
| NM_005288 | TTGATAGCGGATTACACCTACCCCTCCATCTATACCTACGCCACCCTCCT |
| NM_002094 | GTCGGATTTCTGTCCTTGGTACATTGGATTACCGTTTATTCCATATCTGG |
| NM_005513 | AAAGGAAGCATATATAGCAATGGGACAACGCATGTTTGAGGACCTCTTTG |
| NM_004963 | GGTAGCCAGCTATAAAAAAGGCACTCTGGAATACTTGCAGCTGAATACCA |
| NM_000182 | GATTTGAATTCTGACATGGATAGTATTTTAGCGAGTCTGAAGCTGCCTCC |
| XM_033511 | ATACTACCCCTCTGGTTTCCCGGAGTGTTCCACCAGTCAAACTGGAGGAT |
| NM_006825 | AGGACTGCTGTGGACAGTTTGGTTGCATACTCGGTCAAAATAGAAACCAA |
| NM_000867 | GTACCAGAGTCCAATGAGGCTCCGAAGTTCAACCATTCAGTCTTCATCAA |
| NM_006764 | TGCTGCCTTCAAAGCCCGGACCAAGGCTCGAAGCCGTGTGCGGGACAAGC |
| NM_000612 | GGAGGCCAAACGTCACCGTCCCCTGATTGCTCTACCCACCCAAGACCCCG |
| NM_006083 | GTATGGTATCAAAATGTCTGAAGGGCGGAAAACCAGGCGCTTCAAGGAAA |
| NM_006084 | CTGCCCAGCAACGAGTGCGTGGAGCTCTTCAGAACCGCCTACTTCTGCAG |
| XM_045712 | ATTAACACCGAACCCCTGTTTGGCACATTGAGAGATGGATGCCATCGGCT |
| NM_000229 | ATTTCCACACCCAGCTTCAACTACACAGGCCGTGACTTCCAACGCTTCTT |
| NM_000234 | GTGCTCAGGTGGCCTGTTTGTACCGGAAGCAAAGTCAGATTCAGAACCAA |
| NM_025247 | TTCAGCCTTTCAGTCCCTCTCTCTCTGCCTGTGGGAATCTGGACACATTT |
| NM_004255 | AACTTAGACCAACTTTAAATGAACTGGGAATCTCCACTCCGGAGGAACTG |
| NM_133259 | ATCTGTTTCTAAAGCGTTACGCATCTTTGCTGAAGTATGCTGGAGAGCCT |
| NM_000239 | GTGCAAAGAGGGTTGTCCGTGATCCACAAGGCATTAGAGCATGGGTGGCA |
| NM_005909 | CATGAGAAACAGCAAGATCTCAACATCATGGTTTTAGCAAGCAGCAGCAC |
| NM_002379 | CAGGCCCTGACCAGGAAACTGGAAGCTGTGAGTAAGCGGCTGGCCATCCT |
| NM_002396 | TGTATGAATGGCCAGAATCTGCATCAAGCCCTCCTGTGATAACAGAATAG |
| NM_139202 | TGAGGACCCTCCCAGGTTTCCCACTGCGGAACAGGAGTGACTCTGGCTGC |
| NM_002421 | CAAAATGATAGCACATGACTTTCCTGGAATTGGCCACAAAGTTGATGCAG |
| NM_002523 | CGTCGATGTGTTCGGAGGGGCCTCCAAGTGGCCCGTGGAGACGTGTGAGG |
| NM_001862 | AATACCAGCGTCGTCTGGTTTTGGCTGCACAAAGGCGAGGCCCAGCGATG |
| NM_006981 | AACAAGATCACAAGCAGTTTAAAAGACCACCAGAGTAAGGGACAGGCTCT |
| NM_002527 | AATAAACTCGTGGGCTGGCGGTGGATACGGATAGACACGTCCTGTGTGTG |
| NM_002557 | CATTCCTCTGTCAACTCAGTAACCCCTCAAACAAGTCCTCTTTCTCTAAA |
| NM_014735 | CCATGACTCTAGACGGGATTGCCATGGTAAAAGCAAGACACATCCCCTTT |
| NM_032940 | AATACGATCCAGACAATGCCCTGAGGCACACAGTGTACCCCAAGCCCGAG |
| NM_002739 | TGGAACGATTGGAGATCCCGCCTCCTTTCAGACCCCGCCCGTGTGGCCGC |
| NM_134260 | TGGGATTCTCCAAATACTCCATCAGTGTATCCTGTCTTCAGGTGATGCTT |
| NM_003973 | GCATGCAGCTCACTGATTTCATCCTCAAGTTTCCGCACAGTGCCCACCAG |
| NM_006642 | CATGCTGTTAATCAGCTCAAAGATTTGTTGCGCCAACAAGCAGATAAGGA |
| NM_002640 | AAGATTAGGAATGATCGATGCTTTTGACGAAGCCAAGGCAGACTTTTCTG |
| NM_004374 | GGCTGATCAAAGAAAGAAGGCATACGCAGATTTCTACAGAAACTACGATG |
| NM_005850 | CCGGGTGGGATGCCCCATCCAGGGATGTCTCAGATGCAGCTTGCACACCA |
| NM_000578 | GCTGAACAAGGTCGTCACCTCTTCCATCATGGTGCTAGTCTGCACCATCA |
| NM_014251 | GGTGTAACTTTGCTGACTTACGAATTGCTACAGCGATGGTTCTACATTGA |
| NM_006841 | GCGGCATGTGCTTATTGCCGTTGGCCTGCTCACTTGTATCAACCTGCTGG |
| NM_033262 | TGATGGGCCCCCGCATGGTGGATATGAGTTTTCAGAAAGCGCTCCTGTTA |
| NM_014720 | AGCTCATGAAACGCAGGAAAGAGGAGCTTGCACAAAGCCAGCATGCTCAG |
| NM_006938 | CCTGTACAGCTGGAAACGCTGAGTATTCGAGGAAATAACATTCGGTATTT |
| NM_003107 | CCGGGACCTGGATTTTAACTTCGAGCCCGGCTCCGGCTCGCACTTCGAGT |
| NM_012448 | CACAGAACCCTGACTCAGTCCTTGACACCGATGGGGACTTCGATCTGGAG |
| NM_006453 | CTGCGCTTCTGCGTCACGTGGAACACCAACTCGCGGCACTGCCACGAGGC |
| NM_003315 | AAGAAAAAGACTCGCTATGACAGTGGACAGGACCTAGATGAGGAGGGCAT |
| NM_170695 | ATACACAGAGTGGTCTTTTCAACACTCCTCCCCCTACTCCACCGGACCTC |
| NM_005077 | CCGTTCCTGTAAATTGCTACCCGATGGCTGCACTCTCATAGTGGGAGGGG |
| NM_133502 | AGGAGAAGCAGGAGGATGCAGCCATCTGCCCAGTGACAGTGCTCCCTGAG |
| NM_005157 | AGGCATGGGGGTCCACACTGCAATGTTTTTGTGGAACAAGCCCTTCAGCG |
| NM_004418 | CATCTGTCTGGCATACCTCATGCAGAGTCGCCGTGTGCGGCTGGACGAGG |
| NM_001719 | GACGCTGGTCCACTTCATCAACCCGGAAACGGTGCCCAAGCCCTGCTGTG |
| NM_006835 | CAAGACAAGAGGGACATGCTTCCCCTTGTCCACCTTTGCAGCCTGTTTCT |
| NM_000610 | CCAAACACCCAGAGAAGACTCCCATTCGACAACAGGGACAGCTGCAGCCT |
| NM_012287 | CTTGTTACGTTTAGCAAGAATGAATGAAGAGATGCGGGAATCAGAAGGAC |
| NM_003879 | TCTGGCTGCAGCACACTCTGAGAAAGAAACTTATCCTCTCCTACACATAA |
| NM_005197 | TGCCAGTGCCATGATGCTTTTGAATACTCCCCCTGAGATACAAGCAGGTT |
| NM_001882 | GAAAACCCAAATGGAAACAGTATCGGGGAATTCTGTTTGTCTGGTCTTTG |
| NM_001319 | CTCAGGTCTACTACTTCGGTCCGTGCGGGAAGTACAACGCCATGGTGCTG |
| NM_001921 | GTGCAGCAAGATTGTCATTGACTTTGATTCAATTAACAGCAGACCGAGTC |
| NM_006824 | TTTCCTTGAGGGAGATCAGAAACCTCTGGCACAGCGCAAGAAGGCAGGAG |
| NM_003648 | TCTGTGAGTATAAGGACATCTTCACACGGCACGACATCCGGGGCTCTGAG |
| NM_004441 | CCGCAGTGGCTGCGATGGAAGAAACGTTAATGGACACCAGAACGGCTACT |
| NM_002025 | CATACTGCTGGACACTCTGAGCAGAGCACCTTTTCCATCCCAGGACAGGA |
| NM_001530 | ATACAAGGCAGCAGAAACCTACTGCAGGGTGAAGAATTACTCAGAGCTTT |
| NM_005531 | AAAGTGGGAATACCGGGGAGTTGAGATCTGTAATTCATAGTCACATCAAG |
| NM_000874 | GCTATTCACAGGTGCAGTCATAATGCACTACAGTCTGAAACTCCTGAGCT |
| NM_014002 | GAACTGAGGTCCAGGCTGCGGACTCTAGCGGAGGTCCTCTCCAGATGCTC |
| NM_000628 | GCACCTGAAAGAGTTTTTGGGCCATCCTCATCATAACACACTTCTGTTTT |
| NM_000880 | GGAAGGTATGTTTTTATTCCGTGCTGCTCGCAAGTTGAGGCAATTTCTTA |
| NM_001567 | TTCAATAACCCTGCCTACTACGTCCTTGAAGGGGTCCCGCACCAGCTGCT |
| NM_001398 | TGAGCATGCTGCAGACCCAAGACCTCGTGAAGTCGGTCCAGCCCACGACT |
| NM_005544 | GTCTGGCCCGGTGGCTTTCCACAGCTCACCTTCTGTCAGGTGTCCATCCC |
| NM_002249 | GAGAAAGCGACTGAGTGACTATGCTCTGATTTTTGGGATGTTTGGAATTG |
| NM_002755 | ATACGGAATGGACAGCCGACCTCCCATGGCAATTTTTGAGTTGTTGGATT |
| NM_003954 | GAAAGTCCAAATACAGTCTCTTAATGGTGAACACCTGCACATCCGGGAGT |
| NM_002748 | CCTGCTTTTGATACCAATTACTCTACTGAGCCTTGTTGGCAATACTCAGA |
| NM_002408 | GGGGAGATATTAGGGACCATGAACTCTGTAAAAGTTATAGAAGACTGCAG |
| NM_004927 | CAACATCCCCGTCTACAAGGACATCACGCATGGCAACCGGCAGATGACTG |
| NM_004998 | GCAGTCTACCAGTTCAGACCGAGTGTCACAGACGCCAGAGAGCCTGGATT |
| NM_003908 | GACTCTATTTCCTACAGTGCGAAACTTGTCATTCTAGATGTTCTGTTGCC |
| NM_005009 | AGGCTGCCCCAGGAACCATAAGGGGTGACTTCAGCGTCCACATCAGCAGG |
| NM_002542 | CAGGTCATCACCACTTTTATGACCTTTCTCGGACCCCATAGGCTGGATCA |
| NM_012383 | GCACTGCCTTATACTGGGCTTGCCACGGGGGCCACAAAGATATAGTGGAA |
| NM_005746 | TATCTTTACATAGGACGCCAGCAGGGAATTTTGTTACACTGGAGGAAGGA |
| NM_002649 | CGAAGTTTGCAGAGACAAAGGATGGACTGTGCAGTTTAATTGGTTTCTAC |
| NM_021127 | GGCTCCAGCAGAGCTGGAAGTCGAGTGTGCTACTCAACTCAGGAGATTTG |
| NM_006237 | TCCCTGAGCACAAGTACCCGTCGCTGCACTCCAGCTCCGAGGCCATCCGG |
| NM_003479 | ATACCGACCTAAGATGCGATTACGCTTCAGAGATACCAATGGGCATTGCT |
| NM_014369 | TGATCCTGATGGCCTGTCGAGAGATAGAGAATGGGCGGAAAAGGTGTGAG |
| NM_002828 | GTCATTTTGGTTGGCGCTTTTGTTGGCTGGAGACTGTTTTTTCAGCAAAA |
| NM_001469 | AAAGACTGGGCTCCTTGGTGGATGAGTTTAAGGAGCTTGTTTACCCACCA |
| NM_001754 | GCCCTGGGAGGTGGGAGATCTTTGTATAAAAATTGGAACCCAAACTATAA |
| NM_006918 | CCTTTGAGGGGAAGGGACCGCTCAGTTATGTGAAGGAGATGACAGAGGGA |
| NM_004630 | GCAGGGCGAGCCCACGGCCACACTGGCACCTGAGCTGACCTTTGATTTCC |
| NM_003105 | GGGCTATGAGATACACATGTTTGATAGTGCCATGAATATCACAGCTTACC |
| NM_004509 | TCTTGAAGGCCTACTGTCATCCACAAAGCTCCTTTTTTACGGGCATCCCA |
| NM_007237 | TTTGTACAAGACATGCGCCTCATCTTCCAGAACCACAGGGCCTCTTACAA |
| NM_003120 | CACCCCCACCACGTGCACAGCGAGTTCGAGAGCTTCGCCGAGAACAACTT |
| NM_182692 | TGTCAGTTAACTCTGAGAAGTCGTCCTCTTCAGAAAGGCCGGAGCCTCAA |
| NM_001066 | CAGGTCAATGTCACCTGCATCGTGAACGTCTGTAGCAGCTCTGACCACAG |
| NM_006708 | GACCCTCGAGGATTCGGTCATATTGGAATTGCTGTTCCTGATGTATACAG |
| NM_003387 | TCCCTCAGTTCGTCCACGCCCCCGTTACCTTCGCCAGGACGTTCAGGTCC |
| NM_006748 | TTGGGGTAGACGAGTCCCTTTTCAGCTATGGCCTTCGAGAGAGCATTGCC |
| NM_001877 | GGGAAACAAATCCATTCACTGTATGCCTTCAGGAAATTGGAGTCCTTCTG |
| NM_000878 | AGTGGCTCTCTTCGCCCTTCCCCTCATCGTCCTTCAGCCCTGGCGGCCTG |
| NM_005923 | ATGACTTAAAATGCTTGAGACTAAGGGGAGGGATGCTGTGCACACTGTGG |
| NM_000941 | TGTGGGGATGCACGGAACATGGCCAGGGATGTGCAGAACACCTTCTACGA |
| NM_001242 | GGGCCCTGTTCCTCCATCAACGAAGGAAATATAGATCAAACAAAGGAGAA |
| NM_006994 | TCTGCAACAACCAATCAGAACCATAAGCTACAGGCACGCACTGAAGCACT |
| NM_001629 | TTTCCTCGCTGTGCTCTGGTCTGCGGGGCTACTTTGCAGCCAAGTTCCTG |
| NM_002107 | CGGTGCTTTGCAGGAGGCAAGTGAGGCCTATCTGGTTGGCCTTTTTGAAG |
| NM_021603 | GCTGTGGGGGCAATAAGAAGCGCAGGCAAATCAATGAAGATGAGCCGTAA |
| NM_003376 | AACTTCTGGGCTGTTCTCGCTTCGGAGGAGCCGTGGTCCGCGCGGGGGAA |
| NM_000034 | TCCCTTCCCCCAAGTTATCAAATCCAAGGGCGGTGTTGTGGGCATCAAGG |
| NM_005998 | CTGTGAGACCTGGGGTGTAAATGGTGAGACGGGTACTTTGGTGGACATGA |
| NM_002156 | AAGAGAAGGACCCTGGAATGGGTGCAATGGGTGGAATGGGAGGTGGTATG |
| NM_002168 | CCGCGGCGCCACTATGCCGACAAAAGGATCAAGGTGGCGAAGCCCGTGGT |
| NM_000884 | TGGAAGGTGGCGTCCATAGCCTCCATTCGTATGAGAAGCGGCTTTTCTGA |
| NM_005566 | CGGAATAAAGGATGATGTCTTCCTTAGTGTTCCTTGCATTTTGGGACAGA |
| NM_006636 | CTGCAAAAAAGGTGCTGAGGCTTGAAGAGCGAGAAGTGCTGAAGTCTAAA |
| NM_000269 | AAGGACCGTCCATTCTTTGCCGGCCTGGTGAAATACATGCACTCAGGGCC |
| NM_002629 | CGCCTCAATGAGCGGCACTATGGGGGTCTAACCGGTCTCAATAAAGCAGA |
| NM_000365 | TCCTTGTGGGTGGTGCTTCCCTCAAGCCCGAATTCGTGGACATCATCAAT |
| NM_001148 | GATGCTAGTTCTTTGGATTCAAAGACCAAATGCCCAGTAAAAACCCGAAG |
| NM_138271 | CAAAAGGTACAGTGATTGTACAGCCAGAGCCAGTGCTGAATGAAGACAAA |
| NM_004049 | TGCGGAGTTCATAATGAATAACACAGGAGAATGGATAAGGCAAAACGGAG |
| NM_006624 | CAAGAAGCTGGCAACACAGCACAAGCAACTGATTTCTCAGACCAAGAAGA |
| NM_004166 | TGCCAAACCCAGTGGTCCGGGAGTTCAGGATTGCATGAAAAAGCTGAAGC |
| NM_002989 | GCTATCCTGTTCTTGCCCCGCAAGCGCTCTCAGGCAGAGCTATGTGCAGA |
| NM_003542 | TGTGCTTAAGGTTTTCTTAGAGAACGTTATTCGAGACGCCGTCACCTATA |
| NM_012073 | GGGGACAAATGATATGAAGCAACAGCATGTCATAGAAACCTTGATTGGCA |
| NM_000732 | TGCAATACCAGCATCACATGGGTAGAGGGAACGGTGGGAACACTGCTCTC |
| NM_000733 | CCAGAAGATGCGAACTTTTATCTCTACCTGAGGGCAAGAGTGTGTGAGAA |
| NM_001781 | CACATTCTCAATGCCATCAGACAGCCATGTTTCTTCATGCTCTGAGGACT |
| NM_001809 | ACTTCAATTGGCAAGCCCAGGCCCTATTGGCCCTACAAGAGGCAGCAGAA |
| NM_001280 | AGTCAGAGTGGTGGCTACAGTGACCGGAGCTCGGGCGGGTCCTACAGAGA |
| NM_007096 | GGTTATGTCACAAACATAAACCATCCTTGCTACAGCCTAGAACAGGCAGC |
| NM_001891 | CCAAGAACTTCTACTTAACCCCACCCACCAGATCTACCCTGTGACTCAGC |
| NM_001321 | CACAACAGTGGCAATTCACGATGAAGAGATCTACTGCAAATCCTGCTACG |
| NM_001908 | AGTGTACCAACACGTCACCGGAGAGATGATGGGTGGCCATGCCATCCGCA |
| NM_005517 | AAGGGGCATATGTCACTAATAGAATGTCTCCAAAGCTGGATTGATGTGGA |
| NM_001909 | CCGCTGATTCAGGGCGAGTACATGATCCCCTGTGAGAAGGTGTCCACCCT |
| NM_014750 | CTTGATTCACCAGGTCTAAACTGCAGTAATCCATTTACTCAGCTGGAGAG |
| NM_004944 | CAAGGGCCTTCACCAACAGCAAAAAATCTGTCACTCTAAGGAAGAAAACA |
| NM_004089 | AGCCAGCGTGGTGGCCATAGACAACAAGATCGAACAGGCCATGGATCTGG |
| NM_004111 | GGTGAGACCACCAGCCACCTGATGGGCATGTTCTACCGCACCATTCGCAT |
| NM_006851 | TACGGACCAGGAGGGAATTACCCAACTTGGCCATATAAGAGAGGAGCCAC |
| NM_000405 | GAAAAAGCCATCCCAGCTCAGTAGCTTTTCCTGGGATAACTGTGATGAAG |
| NM_002110 | TCCGAGCTCTGGAGCGTGGATACCGGATGCCTCGCCCAGAGAACTGCCCA |
| NM_005524 | CAGTTTGCTTTCCTCATTCCCAACGGGGCCTTCGCGCACAGCGGCCCTGT |
| NM_002136 | GTCGTGGAGGTGGTTTCGGTGGGAATGACAACTTCGGTCGTGGAGGAAAC |
| NM_145904 | GCCACAGCCCCCTTGCCCTCCGCCTGGGATCTGAGTACATATTGTGGTGA |
| NM_005527 | GAGCAGATGTGTAACCCTATCATCACAAAACTCTACCAAGGAGGATGCAC |
| NM_007355 | GTGGTGCTGCTGTTTGAAACCGCCCTGCTATCTTCTGGCTTTTCCCTTGA |
| NM_004258 | GGTACGTGTCTCCAAAGTGTACTGGACCGAAAATGTGACTGAGCACAGAG |
| NM_002194 | ATTTGCCACAGTTGGTGTACCACGTGGAAAATGAGGGTGCTGCTGGGGTG |
| NM_000885 | CTTGTCCAAGACTGATAAGAGGCTATTGTACTGCATAAAAGCTGATCCAC |
| NM_002306 | TTCTGGGCACGGTGAAGCCCAATGCAAACAGAATTGCTTTAGATTTCCAA |
| NM_002348 | AGCCACAGAGGAGTCAGCTGCAAATATTCTCTTCTGTTCTACAGACCTCT |
| NM_005916 | AGAGTCTCTGGCTGACTACATCACAGCAGCATACGTGGAGATGAGGCGAG |
| NM_005520 | TGATGGGAGGCATGGGCTTGTCAAACCAGTCCAGCTACGGGGGCCCAGCC |
| NM_002397 | ACGATGCCATCAGTGTCTGAGGATGTCGACCTGCTTTTGAATCAAAGGAT |
| NM_014791 | CCTGGGTTTACAAAAGATTAGTGGAAGACATCCTATCTAGCTGCAAGGTA |
| NM_000918 | AACGGGGAACGCACGCTGGATGGTTTTAAGAAATTCCTGGAGAGCGGTGG |
| NM_002627 | GGGGGGCTCGCGGCCGGAGCTGATGCCGCATACATTTTCGAAGAGCCCTT |
| NM_006219 | AACAAAGATGCCCTTCTGAACTGGCTTAAAGAATACAACTCTGGGGATGA |
| NM_002654 | CGGAATCCCCAGACAGCTCGTCAGGCCCACCTGTACCGTGGCATCTTCCC |
| NM_000302 | GCTGGACCCTCATGCACCCTGGACGACTCACGCATTACCATGAGGGGCTC |
| NM_002674 | GGTCTGCCACTCAATCTGGCTATAAAAGGATATCAAGCACTAAAAGGATC |
| NM_002140 | AGATCGGATCATTACCATTACAGGAACACAGGACCAGATACAGAATGCAC |
| NM_002702 | TTATTGGAACCCTTCCATGGGTAGTGAACTCAGCTAGTGTGGCGGCCCCA |
| NM_002802 | ATATGACTCCAATTCTGGTGGTGAGAGAGAAATTCAGCGAACAATGTTGG |
| NM_005055 | CTCCGCCATGAGCATCATGACCGAGATCGGAAACCGCCTGGGGCAGGTGC |
| NM_015004 | ATGTCCCCTGCATTGTCACTCTGTGCAAGATTGGCTATCGGCATGTGGTG |
| NM_002970 | GTTGGTTTTGCCATGTACTATTTTACCTATGACCCGTGGATTGGCAAGTT |
| NM_005410 | AAACCTCCCATCTTTATGTAGCTGACAGGGACTTCGGGCAGAGGAGAACA |
| NM_003034 | GTCAGTTAGTGACAGCTAATCCCAGCATAATTCGGCAAAGGTTTCAGAAC |
| NM_005628 | GGCAGGACTCCTCCAAAATTATGTGGACCGTACGGAGTCGAGAAGCACAG |
| NM_004955 | TCCAGCCGTGACTGTTGAGGTCAAGTCCAGCATCGCAGGCAGCAGCACCT |
| NM_031844 | CTCAAGGCCGAGCTCATGGAGCGACTCCAGGCTGCGCTGGACGACGAGGA |
| NM_003091 | CGATTCCTCAGGGCCACGTTACCACAGACCTGTTTGTTTCTTATGCTGTT |
| NM_004780 | GCCCGCAATTTAGAGGGGACATACATGGCAGAAATTTAAGCAATGAGGAG |
| NM_005650 | CCGTGCCCTCTCCCCCCCTTGCAGAACAAGACCGCGAAAGGCAGCCTCAG |
| NM_003249 | AAGCTGCATCCTGAGACCCGGCGGTTCCGAGGATGCCAGCGCCATGCTGA |
| NM_148968 | AGGTGACATGGTCCTGGGACCAGTTGCCCAGCAGAGCTCTTGGCCCCGCT |
| NM_000074 | ATTTGAATTGCAACCAGGTGCTTCGGTGTTTGTCAATGTGACTGATCCAA |
| NM_016292 | TGGACTTGTTGACGACCCTAGGGCCATGGTGGGCCGCTTGAATGAGCTGC |
| NM_021643 | CATCCTTGGTTTTCTACAGATTTTAGCGTCTCGAATTCAGCATATGGTGC |
| BX436497 | AAACTGTTTTTTGGCAGTGGAACCCAGCTCTCTGTCTTGGAGGACCTGAA |
| NM_005347 | ATTGTTCAACCAATTATCAGCAAACTCTATGGAAGTGCAGGCCCTCCCCC |
| NM_003328 | GCTTAGGAAGGAAATGCTACTGAGTGTATGCCAGGATATATGTGAAGGAA |
| NM_007013 | CAAACTGGTGAATTGACAGTTGTGCTTGATGGATTGGTGATTGAGCAAGA |
| NM_021089 | AGCCGGCGGCGTGAACAATCCTCGAGCAGGAACTCACACCTGGTTCAGCA |
| NM_004310 | GGCGACTCTGCTGTGGGGAAAACCTCTCTGTTGGTGCGCTTCACCTCCGA |
| NM_016733 | CAGGACTGTCAACGAAACCTGGCACGGCTCTTGCTTCCGGTGTTCAGAAT |
| NM_002818 | CTCCAAGGAGACTCATGTAATGGATTACCGGGCCTTGGTGCATGAGCGAG |
| NM_005018 | GGAGTATGCCACCATTGTCTTTCCTAGCGGAATGGGCACCTCATCCCCCG |
| NM_002574 | CTGTTGGCCGCTCTGTGGATGAGACTTTGAGACTAGTTCAGGCCTTCCAG |
| NM_031966 | ATGCCACATCGAAGCATGCTAAGATCAGCACTCTACCACAGCTGAATTCT |
| NM_006854 | GCCTCTCATTTTTAGTTAATCACGATTTCTCTCCTCTTGAGATCCTCTGG |
| NM_006332 | GCGGAGAGCTCATGGAAGGCGAGTGGGAACTCGGCTGCCTGCCTTTTTTT |
| NM_004619 | GCCAAGAACGCCTACATTAAAGATGACACTCTGTTCTTGAAAGTGGCCGT |
| NM_016155 | GCCCACAATGACAGGACTTATTTCTTTAAGGACCAGCTGTACTGGCGCTA |
| NM_002659 | GAACGCTCACTCTGGGGAAGCTGGTTGCCATGTAAAAGTACTACTGCCCT |
| NM_000485 | TATTCAGGGACATCTCGCCCGTCCTGAAGGACCCCGCCTCCTTCCGCGCC |
| NM_006763 | TTGGGGAGGACGGCTCCATCTGCGTCTTGTACGAGGAGGCCCCACTGGCC |
| NM_001743 | ATCTCTTGGGCAGAATCCCACAGAAGCAGAGTTACAGGACATGATTAATG |
| NM_006191 | CCAATGGCCCCATGCGGATAACCAGTGGTCCCTTCGAGCCTGACCTCTAC |
| NM_176783 | ACTCAAATCTCTAAGTATTTCTCTGAGCGTGGTGATGCAGTGACTAAAGC |
| NM_002961 | GTAACGAATTCTTTGAAGGCTTCCCAGATAAGCAGCCCAGGAAGAAATGA |

TABLE 28

Polynucleotide probe sequences
for preparation of a large 30mer
nucleic acid array

| GenBank | |
|---|---|
| Accession Number | Polynucleotide probe |
| of Target gene | sequence (5′ to 3′) |
| NM_000246 | CAACAGGATTCACGGATCAGCCTGAGATGA |
| NM_005175 | TTGGCAGCTTGATCATTGGCTATGCCAGGA |
| NM_006559 | GGTGCAAAGATCTCTGTATTGGGAAAGGGC |
| NM_030666 | GGTAGCATCCTATTCTTGGGGAGATTTTCT |
| NM_003955 | TTCCTGGACCAGTACGATGCCCCGCTTTAA |
| NM_018209 | GATGATGGCTGGGACAACCAGAACTGGTAG |
| NM_001165 | GTGTCCTATTTGTAGGAGTACAATCAAGGG |
| NM_014670 | TTGCTGCCTTTACTACTCAAGGTCAGTCTG |
| NM_144578 | ATGCCTGGTGGCCCCCATTCTTACCATTAA |
| NM_144628 | GGCAGTGATGGGGCTGACAGTGGCACTTGG |
| NM_032263 | GGCGAGGCACTATGATACGGAGAGAAATTG |
| NM_006579 | ATGCTGTGAAGCACCTCACTCATGCCCAGA |
| NM_000877 | TTCATCTAAACACCAGTTACTGTCACCAGC |
| NM_002266 | TACACTTTCCAAGTTCAGGATGGGGCTCCT |
| NM_018462 | TTTACACAACACAGGCCACATGGGAAAGGC |
| NM_002444 | GGGCCGAGACAAATACAAGACCCTGCGCCA |
| AB032991 | CAGAAACATGTCTGAAAGTATGGCAGCTGC |
| NM_004762 | AAGAAGGTCTCCTCCACGAAGCGACACTGA |
| NM_000295 | GGTCTGCCAGCTTACATTTACCCAAACTGT |
| NM_030674 | CACACATCAGCAATCTCTCACCACTTCTTT |
| NM_022743 | GAATGCGACGCCAACATCAGAGCATCCTAA |
| NM_003217 | GGCTTCGTCCTTGTTGATACTCAACTCATT |
| NM_022366 | CTGTATGATGAAACCCTGGAAGATAGGTAG |
| NM_021109 | TCCACTGCCTTCCAAAGAAACGATTGAACA |
| NM_014268 | TTCAGAAGAACACGAGGGCCACACAGAAGA |
| NM_003352 | GAACAAACGGGGGGTCATTCAACAGTTTAG |
| NM_001923 | TGGCAGCGGTATGAAGCGAGAGGCCACTGC |
| NM_033503 | AAGCAGAGGTACAGATTGCCCGAAAGCTTC |
| NM_178155 | AACGGTCAAGTACCCCACATATCCTGAGGC |
| XM_088315 | TGTGCGTGGTCTCCAGGTTTCCCTATTACA |
| AK024458 | GCTGGGATCCATGGTTTCCTTACTACAATT |
| NM_014397 | AGAGACCTGACATCGGATACGTGCACCAGG |
| NM_005969 | GATGATGCGGAAATTAACCCCAAGGTGTAA |
| NM_002221 | GGGGCTCAATAACCTCGTCGACATCCTGAC |
| NM_013314 | CCCATGGTGAATAGATCAACCAAGCCAAAT |
| NM_004844 | GCTGACATAAAAATGGTGCAGATTGGCTGA |
| NM_001759 | CCTACAGACGTGCGGGATATCGACCTGTGA |
| NM_001987 | CAGATGAAATCATGAGTGGCCGAACAGACC |
| NM_002827 | GCTTACCTCTGCTACAGGTTCCTGTTCAAC |
| NM_004513 | GATGGACCTGTCACGATTGTCATCAGGAGA |
| NM_001237 | AAGTATCATGGTGTTTCTCTCCTCAACCCA |
| NM_172164 | GAGAATCAGCTGAAACGCGGAGCAGCAGTG |
| NM_000424 | TGGCTTCAGTGCAAGCAGTGGCCGAGGGCT |
| AB037771 | CCAAGCTCTCTTTTCCAGAGAGCAGTGGCT |
| NM_025113 | GACTGTTCAACATGCCTTATGATAACACCG |
| NM_032873 | GGACCAACTGGGGGCTTCAACTGGAGAGAG |
| NM_023037 | GACTCCAGTGTTTCTGGCACTAGTCTCTGA |
| AF509494 | CTTGAGTAAGACAAGTCTGGTGAACGAATG |
| NM_004556 | GCGGAATGTGGAGGATGAGACGCCCCAGGA |
| NM_002984 | GAGTACGTGTATGACCTGGAACTGAACTGA |
| BC025340 | CAGCTGTTGGGGAATTACCGATGCCTTTGA |
| NM_030926 | GCTCCTCATGAACGTGAAGAGGGGGACCTA |
| AF331856 | ACTATACTGACGTCTCCAACATGAGCCGCT |
| NM_000483 | AGGGACTTGTACAGCAAAAGCACAGCAGCC |
| NM_006931 | CCATCCTTCCTGCTATCCTACAAAGTGCAG |
| NM_001888 | CAAACTCATCTATGATTCCTGGTCATCTGG |
| NM_003804 | AGCAGCTTGATTTACGTCAGCCAGAACTAA |
| AB018263 | AACTCCACGCTCACTGCCTCGGAGGTCTGA |
| NM_006230 | TTCTCGGGCTTCGGGGCAGAGGACGATGAC |
| NM_006475 | CAATAACTGAAGTGATCCATGGAGAGCCAA |
| NM_001786 | GGGGATTGTGTTTTGTCACTCTAGAAGAGT |
| NM_031942 | TGAGATACAAGATGGAATGAGGCTGCAGTC |
| NM_001640 | AGGGCATGGAGTATTACCGTGCCCTCAAGA |
| NM_002483 | GTGGCCACCGTCGGCATCACGATTGGAGTG |
| NM_003915 | GCTCCGATTATGACAGTGACGGGTCACATG |
| NM_018842 | CCAGTGAGAAGCTTCAGTGGAGAGGCCTAA |
| NM_147180 | TATCCTTTGAGGAATTCAGTGCTGTGGTCA |
| NM_004924 | ACGGCCTTGTATGGCGAGAGCGACCTGTGA |
| NM_002276 | ATCACTACAACAATTTGTCTGCCTCCAAGG |
| NM_002695 | GTGAGACGGCTGGCAGGTACATCACCTACC |
| NM_003480 | CCTCCCTGTGAAAATGTGGATTTGCAGAGA |
| NM_002023 | CTCCAAGGCAATAGGATCAATGAGTTCTCC |
| NM_006216 | GTGTTATTCATGGGGCAGATAAACAAACCC |
| NM_021813 | TAAGTGTACAACTGACGAACAGCCCAGGAA |
| NM_006565 | GAAGACCAGAATACAGGTGCAATTGAGAAC |
| NM_005537 | CTTTCGGGCGCGGATTTATAGCAGTAGCAG |
| NM_004235 | CCTCGCCTTACACATGAAGAGGCATTTTTA |
| NM_006235 | GCGCTTAACCACACTCTCTCTGTGGAAGGC |
| NM_016269 | TGCATCAGGTACAGGTCCAAGAATGACAGC |
| NM_006164 | TTCTCCTAGTGAATACTCCCTGCAGCAAAC |
| NM_003222 | CCAGCTGATTCTAACAAAACCCTGGAGAAA |
| NM_000700 | GCCAAGCCATCCTGGATGAAACCAAAGGAG |
| NM_019846 | GAAACACGAAACATACGGCCATAAAACTCC |
| NM_032738 | AACAGCATCTGTTGTGGCTATCACAGTCCA |
| BC019046 | TACAGGAACATGATCGGACAGGGGGCCTAG |
| NM_016732 | ACCGGGACGACTTCTACGACAGGCTCTTCG |
| NM_002189 | GACTTGGAAAACTGCTCTCACCACCTATGA |
| NM_004827 | TGGGGCTTGTGGAAGAATCACGTGGCCTTG |
| NM_001830 | CAGGACCCCGAATCCATCATGTTTAATTAG |
| NM_020944 | GGACAGGGCCTATGTTTGGACCAAAGGAAG |
| NM_017784 | TGGAAGCCCAAATATTTTATCCAGGAGGGC |
| NM_002933 | CATATGTGCCAGTCCACTTTGATGCTTCTG |
| NM_022829 | TTGGCCAATGACACATTTCGGACCCTCTGA |
| NM_006399 | AACCTCATGTCAGCTCCCCGCGCTTCCAGC |
| NM_002893 | GAAGAGTCAGATGTCACGACATCCGAACTG |
| NM_003339 | CCTGAGATTGCTCGGATCTACAAAACAGAT |
| NM_005277 | TCCAAAGAGCGGCTCAATGCATACACATAA |
| NM_006820 | TTCTCATCCTCTCTGCACTGAGGCAGATGC |
| NM_024728 | CATCTTCAAGGATCAGCAAGGGTATTACAC |
| NM_025263 | CATCCAGGCGTCAATGGACCTCCTCTGTGA |
| BC046632 | GTGAACCTGAGCGACTCGGACAAGCAGTGA |
| NM_024713 | CCATCTTACTTTGGTTCCAGGATATACTGG |
| NM_014365 | CAGGACAGCCAGGAAGTCACCTGTACCTGA |
| NM_002947 | CCCTCAGTTTTATCCTTTAGGGATTGTGCA |
| AK092000 | CAGTGTACTGAACATACTGTATACCTCGTG |
| NM_022436 | CCAGCTCTTGTCATCCTAGGAATAGTTGTT |
| NM_002967 | ATCCCACACGGTGGCATGCAGGGCGGGTTT |
| BX647445 | CATAAGATTCCAGCTCCTTCAGGTGTTTGA |
| BC022095 | GTCTTGCGAGGACAGCCTCATAGAGAATGA |
| NM_006808 | CACATTTGGGGCAAGTACACTCGTTCGTAG |
| NM_031305 | CGAGAGAGGAAACACAATATGGATTCAGTG |
| NM_014792 | GTTTTCAGGCTGTATCCTTCTACCTCCTGA |
| NM_006304 | CAGTTACGAGCTGAACTAGAGAAACATGGT |
| NM_014686 | AGTGTGGGCCGCAACCAACGACTGCAGTGC |
| AB029034 | AGAGACTCGGCCGTATCCTGAAAATCCACA |
| BQ722784 | ACCCCAGATGGTGAAACGGCCCAAGCCAAA |
| NM_198243 | TAATTACGGAGCAGACACGAACACACGGAA |
| NM_016570 | CGTTTCAGACTTGGATCCTATAAACCTGTC |
| BC001077 | CGCGGTAGACGAGCGGATGCTGCGCTTTCT |
| NM_138379 | GAAGACGAAGACGGCCTTTTTACCCTCTAA |
| NM_024319 | TCTCGCAGGTGGCATGACAGAGGCTCTGAG |
| NM_015670 | GAGCTGTGTCACTGCAAACTCACTGTGTGA |
| NM_015436 | CAAGCTTTATACTTGCCGCTTGTGTCATGA |
| NM_001240 | CTCCTCGAGATCTGGCAATACAGACAAACC |
| NM_001261 | AACCAGACGGAGTTTGAGCGCGTCTTCTGA |
| NM_003095 | GCTTTGTCTGGACATCTGGGTGAAGTTTTA |
| NM_021102 | CGTCTGGAGCTCCGGAGATGACAAGGAGCA |
| NM_003133 | ACAGTCAACTAATGCGACTTATGGTAGCCA |
| NM_006819 | ATGGATGTGGGTCTGATTGCAATTCGGTGA |
| NM_032966 | TGTCGCGGCTCCTGACGAAGCTGGGCTGTA |
| NM_006284 | TGCCCTCAGCGAGTATGGCATCAATGTGAA |
| NM_005648 | GCTGATGGCTGCGAACTTCTTAGATTGTTA |
| NM_003199 | AAATGGACCAACTTCTTTGGCAAGTGGACA |
| NM_003299 | GAACATTGACCCTGATGCAAAGGTGGAAGA |
| NM_006758 | GCGTGACAGGAGGCGGTCGAGAGATCGTGA |
| NM_194259 | GAGAAAAGGGTCCGAGCACAAGCCAAGAAG |
| NM_003366 | CAGCAAGTGGAAATTTGGGACATACACCTT |
| NM_006004 | CGTGGCCCACAAACTCTTTAACAACTTGAA |
| NM_003374 | CACGCGCTTCGGAATAGCAGCCAAGTATCA |
| NM_007048 | TGACAGGTGGAAGTACCTGTGTTATTTCCA |
| NM_006761 | ACGGTGAAGAGCAGAATAAAGAAGCGCTGC |
| NM_005781 | AAGTGCTGGAGATGTTCGACTGGAACCTGG |
| NM_005243 | AGTACTCAGCCGGCAGCATAATGAAAAGTG |
| NM_003902 | GATCCAGCCAGTATAGAACTAGCTCTGTAG |
| NM_006769 | TGAATTCACTTCAGAGCAATCCACTACTGC |
| NM_004689 | AACCGCAGTAACATGAGTCCCCACGGCCTC |
| NM_006875 | TTGGCCTGGTCCTTGCTACCCTAAGCCTGG |
| NM_002953 | CACGTACTCCGCACTCAACAGCTCCAAGCC |
| NM_004965 | CAAAAGTGGAAGCGAAGCCGAAAAAGGCAG |
| NM_000592 | TGAGGCTGCTCTGGGGGCTGATCTGGGCAT |
| NM_178014 | TGAACGACCTCGTCTCTGAGTATCAGCAGT |
| NM_005742 | GCTTGATGACTTAGGGAAAGATGAGTTGTG |
| NM_001901 | AATCGCTGTACTACAGGAAGATGTACGGAG |
| NM_004907 | AGCGTCATCCGAAGTCGCCGATCGCCTGCA |
| NM_003746 | ACCGAAAGGCCGTGATCAAAAATGCGGACA |
| NM_001255 | TGGCGCTGTTTTGAGTTGGACCCTGCGCGG |
| NM_002417 | CTTTGGTGATTCCATTAATGAGAGTGAGGG |
| NM_006209 | TATGCTGCGGAAACTCGTCAGGACAAAATG |
| NM_002162 | ACGTCTATGCAGCCGACAGAAGCAATGGGG |
| NM_000587 | GCATCTCTGTCACCAGCATAAGGCCTTGTG |
| NM_031243 | GGTGGAGGAAACTATGGTCCAGGAGGCAGT |
| NM_003017 | CTCGTAGTCGATCTAGGTCAAATGAAAGGA |
| NM_004994 | CTACGTGACCTATGACATCCTGCAGTGCCC |
| NM_002966 | CTTCTTTTCCCTAATTGCGGGCCTCACCAT |
| NM_001345 | CCCCCCGCTCCACCAATTTCTTTGGCTTCT |
| NM_002497 | GGCTCAAGCCCTGTCAGATATTGAGAAAAA |
| NM_000545 | TGAGTCCGGGCTTCACACGCCGGCATCTCA |
| NM_006184 | TTCTCGAGCGGCTCCCTGAGGTTGAGGTGC |
| NM_000598 | TGTGGATAAGTATGGGCAGCCTCTCCCAGG |
| NM_006152 | CTCCGACACAATGGGCCACCACCAGTGTGA |
| NM_006274 | TCAGCCAAGATGAAGCGCCGCAGCAGTTAA |
| NM_002105 | TGTACACCGGCTGCTGCGGAAGGGCCACTA |
| NM_001428 | TCAATGTCATCAATGGCGGTTCTCATGCTG |
| NM_002415 | AACTATTACGACATGAACGCGGCCAATGTG |
| NM_003234 | GGTGACGTTTGGGACATTGACAATGAGTTT |
| NM_001728 | AAGAACGTCCGCCAGAGGAACTCTTCCTGA |
| NM_002460 | CAGATCTATCCGCCATTCCTCTATTCAAGA |
| NM_003403 | AACATCTGCACACCCACGGTCCCAGAGTCC |
| NM_001344 | GTTTGCCTGAGAATACAGATCAACCCACAG |
| NM_002648 | GATAGGCCAACCTTCGAAGAAATCCAGAAC |
| NM_006585 | GCTATCAAACTCGCTACTAATGCTGCAGTC |
| NM_001826 | ACGAGGAGTTTGAGTATCGACATGTCATGC |
| NM_002466 | CACACATCTCGGACCCTCATCTTGTCCTGA |
| NM_005582 | CTGTGGGATTACAGCCATAGGCATTTTCTT |
| NM_001831 | GCAGGAATACCGCAAAAAGCACCGGGAGGA |
| NM_000038 | AGAGAGGCAGGCGTGAAATCCGAGTCCTTC |
| NM_007295 | TTGCAAGGTCTTACTCTGTTGTCCCAGCTG |
| NM_001226 | CGCAGGTTTTCAGATCTAGGATTTGAAGTG |
| NM_033357 | TGGATGCAGGTACAGTAGAACCCAAAAGAG |
| NM_005190 | GAAGTCAGAACTCTAGCTACAGCCAATCTT |
| NM_001795 | GACTGGGGACCCAGGTTTAAGATGCTGGCT |
| NM_001848 | AGACGATAACAACGACATTGCACCCCGAGG |
| NM_004380 | CAGCCGTTTACCATGAGATCCTTATTGGAT |
| NM_005215 | CCAATGTGTATGAACAGGATGATCTGAGTG |
| NM_004948 | TGGAGTTTCTAGATCACCTGGAACCCAAAT |
| NM_001397 | TGCTGTCGGCGCTGGGGATGTCGACGTACA |
| NM_004448 | CATTGACGAGACAGAGTACCATGCAGATGG |
| NM_001982 | TTCCCCAAGGCTAATGCCCAGAGAACGTAA |
| NM_006137 | ACGAGGACATGTCGCACAGCCGCTGCAACA |
| NM_005245 | AGAGGTGACGATCCCGCCCCTGGATTCCCA |
| NM_004119 | TCTCCGCAGGCTCAGGTCGAAGATTCGTAG |
| NM_182925 | GATAGAGAGCAGGCATAGACAAGAAAGCGG |
| NM_000629 | CAGTTCCCAAACTAGCCAAGATTCAGGAAA |
| NM_001552 | TGTGGACCGGAAGACGGGGGTGAAGCTTCC |
| NM_000572 | GGCTACGGCGCTGTCATCGATTTCTTCCCT |
| NM_000640 | GCCAAACACCTACCCAAAAATGATTCCAGA |
| NM_000564 | CCACTAACTATGAGGTCCTCTGCATTTTCA |
| NM_176786 | CTCAGCAAGGCTCGGTCCTGGACATTCTAG |
| NM_000213 | ACTAGGCCTGGCCGGAGATGTGGCTGAACG |
| NM_001967 | GACCCAAGCACTAGTATTGGCCCCCACCAG |
| NM_004933 | TGTATGGGCACCCGTGCGGGTTGGAGTACG |
| NM_000224 | CACCAACTACCGGTCCCTGGGCTCTGTCCA |
| NM_145110 | CCAGCATGCCCCAGTCCAAAGGAAAATCCA |
| NM_002758 | CTACATACCCAGAGCTAATGCAACATCCAT |
| NM_002412 | AAGCCAGGCTTGGGAGGGAGCTCAGGTCTG |
| NM_004530 | AGCATCAAATCCGACTGGCTAGGCTGCTGA |
| NM_002422 | CTTTCCAGGGATTGACTCAAAGATTGATGC |
| BC063294 | GAGCTTTTCCTTGCAGGAGCGTCAGTTGCG |
| NM_004557 | CCTCCAAGAAATGCCCATAAACCAAGGAGG |
| NM_002514 | CACACCAACTGTCCTAAGAACAATGAGGCC |
| NM_000300 | CACTGCAGAGGGAGCACCCCTCGTTGCTGA |
| NM_001276 | GGCCAGGATCTGCGCTTCCCTCTCACCAAT |
| NM_000535 | GCCAACCTGGGTGTCATTTCTCAGAACTGA |
| NM_015869 | GTGCAGCTACTGCAGGTGATCAAGAAGACG |
| NM_002805 | TGACACCCAATTGCCGGGTGGCTCTAAGGA |
| NM_000314 | CCACTGACTCTGATCCAGAGAATGAACCTT |
| NM_002868 | CAAAGAGTGAACCCCAGAATCTGGGAGGTG |
| NM_002901 | GCTACCAATTACGGGGAAGATCTCACAAAA |
| NM_003821 | CCGGAAATACTTGTGGTTTCTAGATCACCA |
| NM_005617 | ATCCCCTCTGACAGCACTCGCAGGAAGGGG |
| NM_014624 | TTGATCTACAATGAAGCCCTCAAGGGCTGA |
| XM_044622 | GATTGAAGGACCTAGTGTGAGCATAATGGA |
| NM_000602 | GGAACAGTCCTTTTCATGGGCCAAGTGATG |
| NM_003012 | TGCCCCACCTTTCAGTCCGTGTTTAAGTGA |
| AF311912 | ATCACCTCGGTGAAGCGGTGGCAGAAGGGG |
| NM_139276 | ACCTCGGAGTGCGCTACCTCCCCCATGTGA |
| NM_003220 | AGCCACACGGACAACAACGCCAAAAGCAGT |
| NM_003225 | ATCCTAATACCATCGACGTCCCTCCAGAAG |
| NM_003842 | AAGAATCAGGTACAAAGCACAGTGGGGAAG |
| NM_000546 | CTCCCGCCATAAAAAACTCATGTTCAAGAC |
| NM_138764 | GGTCTATAATGCGTTTTCCTTACGTGTCTG |
| NM_001328 | GATAGAGACCACGCCAGTGACCAGTTGTAG |
| NM_058197 | GGAGTTTTCAGAAGGGGTTTGTAATCACAG |
| NM_000594 | CCCAGGGACCTCTCTCTAATCAGCCCTCTG |
| NM_003810 | CAGTTTTTTCGGGGCCTTTTTAGTTGGCTA |
| NM_002609 | AGGTTGCTGACGAGGGCCCACTGGAGGGTT |
| NM_001069 | CACGGCCGACGAACAAGGGGAGTTCGAGGA |
| NM_003005 | ACAAACGCTGCATTTGACCCGAGTCCTTAA |
| NM_002985 | AAAGAACCGCCAAGTGTGTGCCAACCCAGA |
| NM_000964 | GCATATGTTCGGGGATTTGGTGTGCAGTTG |
| NM_024408 | CACATGTCTGAGCCACCACACAACAACATG |
| NM_001465 | GAGAGAAACCTAAAGTCTCTGACTCAGACA |
| NM_001618 | GACCGGGATTTCATCTGGTGTGATAGACAC |
| NM_004322 | TCCAGTCCTGGTGGGATCGGAACTTGGGCA |
| NM_021960 | TAGTTAAACAAAGAGGCTGGGATGGGTTTG |
| NM_004383 | TGAGCACATCAAAACCCACGAGCTGCACCT |
| NM_015583 | TGGAGGGAGGTTTTCAGCCCTGGTATATGA |
| XM_373407 | TGTCGGGGAGTACCTTCAACTGGCCCTACG |
| NM_016734 | GCTCCCCCTACTATTATAGCGCTGCCGCCC |
| NM_000808 | GCCACATATGTCAACCGGGAGTCAGCTATC |
| NM_002964 | GGCTGGCAGCCCACAAAAAAAGCCATGAAG |
| NM_002965 | AGAAGATGCACGAGGGTGACGAGGGCCCTG |
| NM_001250 | AGAGAGTCGCATCTCAGTGCAGGAGAGACA |
| NM_003407 | GACTCCCCATCTTCAATCGCATCTCTGTTT |
| NM_003674 | GAGGGCCAGAGCAAGCGCTGTAAACCCTGA |
| NM_078467 | CTCCAAACGCCGGCTGATCTTCTCCAAGAG |
| NM_006145 | ATCAAGAACCGTACTTGAGCAGGTTCTTCC |
| NM_000122 | CACCCGCTCTTCAAGCGCTTTAGGAAATGA |
| NM_006712 | ACCCTTCGAGGAACTGGAGTCCCAGAGAGG |
| NM_000175 | TTGCCCTGTCTACTAACACAACCAAAGTGA |
| NM_000162 | ATGCTGGACGACAGAGCCAGGATGGAGGCC |
| NM_000852 | CTCCCCATCAATGGCAACGGGAAACAGTGA |
| NM_005526 | CCCAAAGCCAAGGACCCCACTGTCTCCTAG |
| NM_001540 | GCAAAATCCGATGAGACTGCCGCCAAGTAA |
| NM_002432 | AAACTGGTGTGTGGAAGTCACAGCTTCATC |
| NM_004146 | AAGGCGGCAGAGTTGGCCAAAGGCCAGGGA |
| NM_002505 | GTCCAGACCCTCCAGGTAGTCCAAGGGCAG |
| NM_002511 | CACAGCATGAAGCAGGAAATGGCAATGTGA |
| NM_006170 | GCCTTCTCTACTGTGCATACCCATGAAATT |
| NM_002910 | AAAGGCTGCTTCCACGTGCCGCGGTGCCTA |
| NM_005281 | TTCCGATCCCGCTCCCCCAGTGATGTCTAG |
| NM_005981 | CCTAGAGCCAACCCCAGTGCCTTTCTATGA |
| NM_003410 | AAAGTGGATGGCACTTGCCCTGAGGTCATC |
| NM_007146 | TGCCTTTCTTGCCCCAAGCTTTGCCTACAT |
| NM_002419 | TTGATCCCTGGAGCTTTGTGTCAGCTGGGC |
| NM_001197 | TGAGGTTCTGGAGATCCCCGAACCCCGGGT |
| NM_000418 | CTCCGTGGGACCCACATACATGAGGGTCTC |
| NM_006254 | CAAGAGGCTGGGAATGACGGGAAACATCAA |
| NM_002166 | CACGGATATCAGCATCCTGTCCTTGCAGGC |
| NM_033306 | CCACCATAGAACGACTGTCCATGACAAGAT |
| NM_020250 | ACAGGTGTCTGTATTGCTCTACCTACCTAA |
| NM_000249 | CCTGGCTCCATTCCAAACTCCTGGAAGTGG |
| NM_002228 | TGATAATCCAGTCCAGCAACGGGCACATCA |
| NM_001654 | CTACTCAGCGCAGCCCGCCTTGTGCCTTAG |
| NM_004047 | TTGGGGTCATCGTCGCAATTCTTCAGACCT |
| NM_000729 | AGTGCCGAGGAGTATGAGTACCCCTCCTAG |
| NM_006273 | ACTGTCCCCGGGAAGCTGTAATCTTCAAGA |
| NM_004073 | GCTTGTACTTACCTCGCTTCCCACCTTCGG |
| NM_006268 | TCTCACCCCGTCCATGTCTGAGCCCCCTGA |
| NM_002104 | AGCAACCTTGTCCCGCCTCATACAAATTAA |
| AK127132 | AGTGGGTAAAGACCATACGTTGGAGGATGA |
| NM_004422 | TGGCCATGGGCAATCCCAGCGAGTTCTTTG |
| NM_006014 | TGCCGAGCCCCACCAAAGGGTGGTTGGGAA |
| NM_003824 | GTGACCTCCAGAACAGGAGTGGGGCCATGT |
| NM_002055 | GAGAGGTCATTAAGGAGTCCAAGCAGGAGC |
| NM_005313 | AGCTACAAACCCCCCTGTAATTCAAGAAGA |
| NM_001513 | CCAGATACACCCACTGAGCTGAGGGCCTAG |
| NM_003883 | GCCAAGACCGTGGCCTATTTCTACGACCCC |
| NM_005339 | ACCCATTTAATCCCCCTAAGGTCCGGTTTA |
| NM_003549 | AGGGCCCCCTTCAAAAAGCCTGCTGGAGAC |
| NM_004763 | CTCTGAAGACCACAGATGCAAGCAATGAGG |
| NM_002301 | GCGGAATGGTGTCTCAGATGTTGTGAAAAT |
| NM_004993 | CTGACATACGAGCTCCATGTGATTTTTGCT |
| NM_002790 | CCTGGCCAGAATTTCCACATGTTCACAAAG |
| NM_002971 | CGGTTTAGAGGTCGATGTGGCAGAATATAA |
| NM_003769 | CACTACTTCTCTCCTTTCAGGCCCTACTGA |
| NM_005835 | CCACCAGGAAAGGTCCAGATTTCTGTTCAT |
| NM_181314 | CCCACGTCTGGCCTTCTGTCCACTGTGCTG |
| NM_003262 | ATCGATCCCAGCACAGTAGTGGAAATGGAA |
| NM_006497 | GGGACCATCGACCGTTTCTCTCCCACCTAG |
| NM_007117 | CTGAAAAACGCCAGCATCCGGGCAAGAGGG |
| NM_006759 | TCTGGAAACCTTCGCATCTTGGACCACTGA |
| NM_014232 | ATCTTGGGAGTGATTTGCGCCATCATCCTC |
| NM_006299 | AGCTTTAATCGACACTGCAACCTCATTCGC |
| NM_000072 | CAGTGTTGGTGTGGTGATGTTTGTTGCTTT |
| NM_002211 | CCAAACTACGGACGTAAAGCTGGTCTCTAA |
| NM_004242 | AGGAAGGTACTGCACCATCTGAAAATGGTG |
| NM_005589 | GCTGTTGTCATGCCTACCATGGGCCGTTAG |
| NM_022067 | TCCAGTCCCTTTCTGATGCTCTGTCAGACA |
| NM_001752 | AGTCCGGATCTCACTTGGCGGCAAGGGAGA |
| NM_001793 | TGTTCGACTATGAGGGCAGCGGCTCCGACG |
| NM_003159 | GACATACCATGAGAATGCGGCACTGACGGG |
| NM_156039 | CCCAGGCGATCTGCATACTTTAAGGACCAG |
| NM_004941 | AGAATATCTCGAGCTTTCCGACGGCGCTGA |
| NM_021071 | CATCTCTCTCCTTTTTGACCAGTGTCATCA |
| NM_005665 | AACAGAAGACAGTGTGCTGGAGACCAGAGA |
| NM_004475 | ATCAAGAAGGCCACTGGTGTGCAGGTGTGA |
| NM_002133 | ACAGTTGCTGTAGGGCTTTATGCCATGTGA |
| NM_004125 | GGCTGGCGTGGAGAGGATCAAGGTCTCTCA |
| NM_002102 | GGCTCGTGTTATTTTTGAGGTGATGCTTGT |
| NM_005318 | TCCAGTGCCAAGAGGGCCGGCAAGAAGAAG |
| NM_002108 | CAACAGCCTTTTCACTGCAATTTCTGCACA |
| NM_006644 | CCCCTTCACCTGAACTTACCTCAGAAGAAA |
| NM_181430 | GCCAGCTCAGACTGGAGTTGCCTCAGTTGA |
| NM_014780 | AGATTCGCTCCCGGGGTGTGCCCTATGCCT |
| NM_000917 | GGAAAATTGACCACAGCACAGTACAGAGTA |
| NM_000869 | CCCACCTCCTGCATGTGTCTCTTGCCTCTG |
| NM_006218 | CATGGTGGCTGGACAACAAAAATGGATTGG |
| NM_005084 | GGGACCAACATTAACACAACCAATCAACAC |
| NM_002890 | CGCTCCTCCAACTAACCAGTGGTATCACGG |
| NM_134269 | GAAGGGGCTGGTAAAAACCAAAAAGTCCTA |
| NM_003242 | GAAGACGGCTCCCTAAACACTACCAAATAG |
| NM_003451 | AATGCACAAGCGAATCCACAATGGCCAGAA |
| NM_019841 | ACTGAACCTTAGTGAGGGGGATGGAGAGGA |
| NM_000416 | GCTGCTTTACTACTCTTTCTAGTGCTTAGC |
| NM_003177 | GTCAGCGGGTGGAATAATCTCAAGAATCAA |
| NM_000277 | CCAGCAGCTTAAGATTTTGGCTGATTCCAT |
| NM_053056 | TGATCAAGTGTGACCCAGACTGCCTCCGGG |
| NM_000873 | GTGTTGCTGTCCCTGTTCGTGACATCTGTC |
| NM_001092 | AGGTTCTGGAGGACGAAGATGTGTTTCTCC |
| NM_004454 | CCCACTTTGAAGACAGCCCCGCTTACCTCC |
| NM_005225 | CTCATCCCTCACCACAGATCCCAGCCAGTC |
| NM_001760 | CCTACAGATGTCACAGCCATACACCTGTAG |
| NM_004064 | GACGGTTCCCCAAATGCCGGTTCTGTGGAG |
| NM_000075 | CTACATAAGGATGAAGGTAATCCGGAGTGA |
| L27560 | TCACTCCTGAGAGGGCTCCGGCCCAGAATG |
| NM_002467 | CTTGTTGCGGAAACGACGAGAACAGTTGAA |
| NM_001175 | CTGTCGATTAAGAAGGAGTGGACAGAATGA |
| AB007921 | GGTGCCTGGGATTTTGACTGGGAGATATAG |
| NM_000724 | TAAATACATTATTCCTGGGGGTTCGGCAGA |
| NM_002987 | TGCTCCAGGGATGCCATCGTTTTTGTAACT |
| NM_001803 | CTTCGTGGCCAATGCCATAATCCACCTCTT |
| NM_014376 | AGACCAGTGCTCACTATGAAGAGAACAAGT |
| NM_182908 | TTGCGGTGGCAGGCTACTCCACTCGGCTCT |
| NM_004434 | ACAAGCATCATGCAGTGGCGCGTCATTTAG |
| NM_014210 | GCTCACAGCTACAAGGGAAAGAAAAGATGA |
| NM_013451 | CAGCCCAACATTTCCAGATACTACCTGCGT |
| NM_003088 | TTCGAGTTCTGCGACTATAACAAGGTGGCC |
| NM_000156 | ATCATGTTTGAGGAGACGCAGGTGCCCGCG |
| NM_002048 | TGAGGACTACGATGACGAGCAGCGCACCGG |
| NM_002051 | TGGACCACACCACCCCTCCAGCATGGTCAC |
| NM_000169 | ATCCCACAGGCACTGTTTTGCTTCAGCTAG |
| BM722299 | GTGGTTCACAACGACAGGCTACTGAGCACC |
| NM_002224 | CAGGCAACGCCTAGGCTTTGTGGATGTCCA |
| NM_005335 | CTTCCCTGCAAATTATGTCAAGCTTCTGGA |
| NM_002167 | CGGAACTTGTCATCTCCAACGACAAAAGGA |
| NM_004633 | TGGATGCACAGACGGTGCAAACACAGAACT |
| NM_015187 | TACTATCGCCTTCGCAGTCTGCACCCCGCC |
| NM_015196 | AGAGACTCGAGTTACTGTGGGAATGTGTGA |
| NM_005556 | GAAGGCTTATTCCATCCGGACCGCATCCGC |
| NM_005356 | CCCATAGTCCGACTGGATGGGAAGGGCAGG |
| NM_005933 | ACTGGGGCAAGAACGGAGTTGCTTCTATAA |
| NM_005098 | GACTGGAACTCACTTGATGGGATTATTCGT |
| U50748 | GCCTCAATTCCAAACTTGTTCTACTCAGAC |
| AB033114 | TAGGAGAAGCACTGTTGTTTTCCACACAGT |
| NM_002499 | CCCACCTGGAAGGACTAATGAAGGACCTAA |
| NM_004289 | CTTGATAGTACCCAAAGAACTGGTGGCCTC |
| NM_005654 | TTCAACTGGCCTTACATGTCCATCCAGTGC |
| NM_020992 | TATGAAGTGGTCACTGTGTTCCCCAAGTGA |
| NM_003311 | TGAAATCCCCCGACGAGGTGCTACGCGAGG |
| NM_002661 | CAGTGTTAATGAGAACCACTCCAGCTGTAC |
| NM_015568 | AAGGTGCATGGCTGTTGCCGTATCTCCTAG |
| NM_006115 | GTGGGGACAGAACCTTCTATGACCCGGAGC |
| NM_080588 | TCCAGACGGCAGAGCAGTACCAGTTCCTGC |
| NM_021038 | CGGCTGTCAAATCACTGAAGCGACCCCTCG |
| NM_006498 | GCGTAAGGGGCGGGTTCAACATGTCCTCTT |
| NM_014904 | AGGGTAATGGAAGAAACGCCCAGTATTCTC |
| NM_002972 | TCTCGTTACCGACCACTAGGGGTGGCAGGG |
| NM_006142 | TGGAGACTGAGCTCCAGGGCGTGTGCGACA |
| NM_003569 | AGTCTCATCATATGGGGATTGAACCACTGA |
| NM_015055 | ACCCTCACCTGATCACTAACTGGGGACCTG |
| NM_015173 | ACCTTAGAACTGGAGCGGTCGGCCCTGCTG |
| NM_016021 | GCATTGGCAGCTCTTATATTCCGACGAATA |
| NM_005573 | GAGGAGGTTGCTCAAAGAAGTACAGTCTTT |
| NM_031407 | TCCCCTTTCCATGCCGTCCATGATCCCCAC |
| NM_004665 | GACCAGCAATTCAGCAATAACTTACCTGCT |
| NM_001783 | AGGAGGGCAACGAGTCATACCAGCAGTCCT |
| NM_000626 | ACAGAGCTGCGAGTCATGGGATTCAGCACC |
| NM_006762 | CAGATTGATCAAGTGCATGAACTCGGTGGA |
| NM_002838 | CACCATCACAGCGAACACCTCAGATGCCTA |
| NM_173216 | CTGCCTGGCTTCCGGACCATTCACTGCTAA |
| NM_002163 | GCCTCACACCAGAGATCATTTTTCAGAGAA |
| NM_002339 | GGAAGTATGAGAAGGTGCTTGTGGAAGGGG |
| NM_152866 | CCTCACCAATAGAAAATGACAGCTCTCCTT |
| NM_006495 | CTCTATTCCTCCCAACTCTGATCAAGATCT |
| NM_005246 | ACCTGAAAATCGCCCTAAGTTCAGTGAACT |
| NM_000698 | AGCCAGTGGTTCGCCGGCACTGACGACTAC |
| NM_004271 | CTGAAAAACGGTCCACCGTGGCCTGTGCCA |
| NM_001715 | GATGAGCAACCCCGAGGTCATCCGCAACCT |
| NM_003254 | AACTGCAGAGTGGCACTCATTGCTTGTGGA |
| AK090461 | TCCCGACGGTGGGTTTGTTCTACTGGGGCC |
| NM_004071 | CTCTCAGAGAAGCCTTAAAGCATCCTTTCT |
| NM_000757 | CGTTCCTTTGACTGACACAGGCCATGAGAG |
| NM_020548 | TCTGCCAATCCGGGCACTGGGACAGAGGCT |
| NM_005232 | GAGGCTGGTCTAATTTTCCTTCCCGGGAGC |
| NM_002017 | ATCAATCAGCCAGTGAGGGTCAACGTCAAG |
| NM_000206 | CCATGTTACACCCTAAAGCCTGAAACCTGA |
| NM_004559 | GCTGAGAATTCGTCCGCTCCCGAGGCTGAG |
| NM_021103 | ACGCAGGAGAAGAACACCCTGCCGACCAAA |
| NM_138933 | GCAGTCCATCATTTTGCAAACTTTGCTGGA |
| NM_001614 | TGGAAGAAGAGATCGCCGCGCTGGTCATTG |
| NM_005919 | AAGACCTTCCCCTATCCCTTGCTCCTCGCC |
| NM_000026 | TCTTTCACTGGTCGTGCCTCCCAGCAGGTG |
| NM_000687 | CCCTTCAAGCCGGATCACTACCGCTACTGA |
| NM_001621 | CCTGATTTGACATCCAGTGGATTCCTGTAA |
| NM_001647 | TTGGCAAGAAACCCTAATCTCCCTCCAGAA |
| NM_001664 | CAAGCTAGACGTGGGAAGAAAAAATCTGGT |
| NM_152862 | ATCACGGGGAAGACGTTTTCATCCCGCTAA |
| NM_005171 | TCAGACTAGCAGCGGACAGTACATTGCCAT |
| NM_001519 | GTGTCATACCACGCCGACGAGGAGGCTGAC |
| NM_000900 | GCCATGGTTTATGGATACAATGCTGCCTAT |
| NM_001746 | AGAAGATGGTGGCACTGTCAGTCAAGAGGA |
| NM_004347 | ACCATAGAACGAGCAACCTTGACAAGAGAT |
| NM_005201 | CGTTCCTCCAGCGTAGACTACATTTTGTGA |
| NM_000560 | GACAAAACCAGCCAGACCATAGGGCTATGA |
| NM_004356 | AGGAGGACTGCCACCAGAAGATCGATGACC |
| NM_001769 | GCTATCCGCAGGAACCGCGAGATGGTCTAG |
| NM_001827 | GAGTCTAGGCTGGGTTCATTACATGATTCA |
| NM_000397 | TCTCCAACTCTGAGTCTGGCCCTCGGGGAG |
| NM_004728 | GAAGCGGAGTTTCAGTAAAGCATTTGGTCA |
| NM_021907 | ACAAGGTACGAGGAGAAACCTCCGCAATGA |
| NM_006169 | AAGGACTTTTCTCCCTGGTGGCGAGGAAGC |
| NM_004416 | TGGCAGGGATGACCGGGATACTGCTGTGCG |
| NM_001948 | CTTACGTCTCTGCTTCGCTCAGCGATGCAA |
| NM_001951 | CCCCGGCAGATGACTACAACTTTAATTTAG |
| NM_001961 | TCCTGCCCGGAGACCCCTTCGACAACAGCA |
| NM_001412 | CATACGGCGAGCTTCCAGAGCATGCTAAAA |
| NM_003752 | AAACGAGGGCTACATGCGCCGCGGTGGCTA |
| NM_001436 | ATGCCGTGGTCGTGGGAGTGTACAGGCCAC |
| NM_005252 | AGCTGCACTGCTTACACGTCTTCCTTCGTC |
| NM_000146 | TTCGAAAGGCTCACTCTCAAGCACGACTAA |
| NM_012197 | ACCGGGGTTCAAGGGAAAGAGACTTGCTGA |
| NM_000435 | GCCGGAAGTTACCCCCAAGAGGCAAGTGTT |
| NM_005890 | GACGATGTTTCTTGCCTCTCTCCCCAGAAC |
| NM_002065 | ATGAAACCGGCGATGAGCCCTTCCAGTACA |
| NM_002080 | CTCCAGCAACGTGGGCTACCTTGCCCATGC |
| NM_002092 | GGTCCCACGTTCATCATAGGTATATTGAAC |
| NM_004493 | GTGATGACCATTGCCCCAGGTCTGTTTGGC |
| NM_004964 | GACAAACGAATTGCCTGTGAGGAAGAGTTC |
| NM_004494 | CCAGGCATCAGAGATCATGAGAGCCTGTAG |
| NM_002129 | GCTGCATATCGTGCCAAGGGCAAAAGTGAA |
| NM_031266 | GCCCCGGCTACGACTACAGTCAGGGTAGTA |
| NM_014413 | GATGACGGAAAGGATGGGGGCGTGGGATGA |
| NM_002568 | CGCCCAGCTGCTCCTAGACCACCATTTAGT |
| NM_198336 | ACATAGGACGACAGTTAGCTATGGGTGTTC |
| NM_000212 | TTCACCAATATCACGTACCGGGGCACTTAA |
| XM_290793 | AGCGGGGAAGGAATGAAGCGTGTCCTGACT |
| NM_005956 | TCTGTGGCCAGTGAAATTATGGCTGTCCTG |
| NM_005962 | ACGCCTCTTCATTCCCGTCCATGCCGAGCC |
| NM_000265 | ACCAAGCGGAAGCTGGCGTCTGCCGTCTGA |
| NM_005601 | ATCCTCTTGCTCTGTACAGGTGCCCTGAGC |
| NM_002512 | GTCTTGTGCTCATGACTGGGTCTATGAATA |
| NM_000270 | AGCATTCCACTCCCTGACAAAGCCAGTTGA |
| NM_004152 | CTACACGTTCGAGAGAGAGTCTTCGGGAGA |
| NM_001604 | CCCATGCAGATGCAAAAGTCCAAGTGCTGG |
| NM_002541 | AAGCCAAGACTTCGGACCACCATCAGCCGC |
| NM_013232 | TACCTGTCCATGGTCTTCAGTATCGTATGA |
| NM_000291 | CCTGGGGTGGATGCTCTCAGCAATATTTAG |
| NM_021129 | AGACGTGGATAAGTGGTTCCATCACCAGAA |
| NM_001198 | GCCACCAACAGTGAAGAGGTTATTGGAGTG |
| NM_006406 | GGAAACCTGGTAGTGAAACAATAATCCCAG |
| NM_002836 | TTCAGGTAATTCTGACTCGAAGGACAGAAG |
| NM_002871 | GTGGCCTTGGAACGAGTTTCCCATGAGTAA |
| NM_002881 | TCAGCGAAGACCCGGGCCAACGTGGACAAG |
| NM_005777 | GCTGTTCGAAGAGTCATGTTTGCTCGATAT |
| NM_004851 | CTTCTTGGGGACGTATGTGGCCGTCTTCGA |
| NM_004159 | GCTGCGCCTTTAGATGACACGACCCTACCC |
| NM_002923 | CAAATCACCACAGAGCCTCATGCTACATGA |
| NM_000976 | AGTGGTGCTGTGGAATGCCCAGCCAGTTAA |
| NM_000980 | CGCTACGAGAGTACAAGGTAGTGGGTCGCT |
| NM_000983 | ACTGGTTGCGCGTAGTTGCTAACAGCAAAG |
| NM_000969 | AGAAGAAGGATCGGGTAGCTCAAAAGAAGG |
| NM_000970 | CGATCTGTGTTTGCTCTGACGAATGGAATT |
| NM_000661 | CTTGTTCAGTATCTCAAGCCCAGAAAGATG |
| NM_002951 | GTTGGCCCTGACAATCATAGCCAGCACCTG |
| NM_001014 | TGGAGGGTGAGCGACCTGCGAGACTCACAA |
| NM_001021 | CTTTTGGACTTCGGCAGTCTGTCCAACCTT |
| NM_001028 | ACCAGAAATACCAAGGGTGGAGATGCTCCA |
| NM_001031 | TCAGAGCGAGAAGCCCGGAGGTTGCGCTGA |
| NM_001007 | GAGACAAAAGACTGGCGGCCAAACAGAGCA |
| NM_001009 | CAAGGGCTCCTCGAACTCCTATGCCATTAA |
| NM_001011 | CGGGCAAGGATGTTAATTTTGAATTCCCAG |
| NM_001013 | GTCCTTCATTGTCCGCCTGGATTCCCAGAA |
| NM_003011 | CACGACGGGGCCGACGAGACCTCAGAAAAA |
| NM_006427 | GTGGGCAGATGCTGATTGGACCAGACGGCC |
| NM_001636 | CATGTACACGGGCACCGTCGACTGTTGGAG |
| NM_006516 | CATCCCCTGGGGGCTGATTCCCAAGTGTGA |
| NM_002860 | TTACTACTAAGTGGCTGCTGCGAGGGAAGG |
| NM_003134 | TAGAGCTAACATGGATGGGTTGAAGAAGAG |
| NM_006280 | CTTGGCCTTCAGTGCGAAGAGCCACATCCA |
| NM_003153 | CCACATGGACCTAAGGGCCAACCCCAGTTG |
| NM_005801 | GACGATCAGCTGAAGGTTCATGGGTTTTAA |
| NM_003258 | CAGATTCTGCAATGCAGCCCTGCCAACTGA |
| NM_014220 | CATATGTGGCTTTTGCTGCTCTCACCAACA |
| NM_003318 | GGTGGTGAAAGTCATAATTCTTCATCCTCC |
| NM_006000 | AGGTGGGCATCGACTCCTATGAGGACGAGG |
| NM_006009 | GCGTCCAACCTATACTAACCTGAATAGGTT |
| NM_006002 | GCGACCCTGATGAACTAAGATTTAATGCGA |
| NM_006508 | GCTGAGTTGCCCAAGGCCCAGATCAGCTGC |
| NM_000375 | AGTTTGCAGCCATCGGCCCCACTACGGCTC |
| NM_003380 | GAAACTTCTCAGCATCACGATGACCTTGAA |
| NM_000377 | GGAGCGCTTTTGGATCAAATCCGGCAGGGA |
| NM_005080 | AGACTACGTGCACCTCTGCAGCAGGTGCAG |
| NM_153380 | ACCTTCACCTGGAAGTCACGCCTCAATATA |
| NM_012203 | AGGGGAGCCGATGCCTAGTGAACTCAAGCT |
| NM_001101 | TCGAGCAAGAGATGGCCACGGCTGCTTCCA |
| NM_001785 | GGTACGTATATTGTCATGACGGTCCAGGAG |
| NM_002341 | AACATCAGTCACCCCGATATGGTGGACTTC |
| NM_001152 | CAAGGGTGCATGGTCCAATGTTCTCAGAGG |
| NM_144488 | GAGGCATGTACCTCACTCGCAACGGGAACC |
| NM_021966 | CTACCCTGATGGACGATACCGATCCTCAGA |
| NM_002350 | GAGATCCAACGTCCAATAAACAGCAAAGGC |
| NM_001774 | GTCTACAACCGGCTCGCTCGATACCGTTAG |
| NM_013230 | CTCACTCTCTCTTCTGCATCTCTACTCTTA |
| NM_006325 | AGAGGTTGCTCAGACAACTGCTCTCCCGGA |
| NM_005340 | CTTGGAGGTCGGCAAATGCATTGGCCTCCT |
| NM_003039 | GGCAACCCTGATAGCTGCCTTTGGGTCATC |
| NM_002046 | GTGGACCTCATGGCCCACATGGCCTCCAAG |
| NM_000334 | AGACTGTGCGCCCAGGTGTCAAGGAGTCTC |
| NM_000484 | AAGTGGTATAAGGAAGTACATTCTGGCCAG |
| NM_170662 | CCTCCAGTATCCCCACGTCTAAATCTATAG |
| NM_006904 | GAGAAGGCGGCTTACCTGAGTGATCCCAGG |
| NM_133480 | CACTTAGTGCCCACAACCGCACCAAGAAGC |
| NM_000633 | ACCCTGGGTGCCTATCTGGGCCACAAGTGA |
| NM_002086 | CCGAGAGAGTGAGAGCGCTCCTGGGGACTT |
| NM_005248 | GACTACTTCACCTCCGCTGAACCACAGTAC |
| NM_002447 | TCTCACCCATGCCAGGGAATGTACGCCGGC |
| NM_003656 | TGTTGCTGTCGAGACTGCTGCGTGGAGCCG |
| NM_138980 | GACCCTGGCATCTGACACTGACAGCAGCCT |
| NM_016072 | CCTTGGATCCCTCCTAAATTTACCTGGAAT |
| NM_000785 | CTGGTACCTGAAAGGAGCATCAACCTACAG |
| AF200348 | TATGAGAACCCTGGGGTGTTCTCCCCGGCC |
| BC060842 | GGCTACCCATACAGCTTTCTGATGAGTTAA |
| NM_006302 | GACCAGCCTTGTCTTACTGGCCATGGCTGA |
| NM_020199 | CCTAGATCAGAATGTTAATGAGGCAATGCC |
| NM_024006 | GTTTCCGGAAGGTCCAAGAACCCCAGGGCA |
| NM_002229 | CTGCTTGGGGTCAAGGGACACGCCTTCTGA |
| NM_024099 | CTAGTGAACCACAGCTCAAAAGGCAAAAGA |
| NM_006378 | TGTGAGCTGAAGTTCGCTGACTCAGACGCA |
| NM_033334 | GCACTATTTCTGTTTCAGATGATCGGGCTG |
| NM_002814 | GGCCAAAGGTGGCCTGGGTTTAATACTCAA |
| NM_017771 | GATGCTCATGTAAGCAGCTTTTCGAGAGAA |
| NM_003002 | CAACTATCACGATGTGGGCATCTGCAAAGC |
| NM_003627 | GTGCTTAGCGGCTCTGAGGTGACCGCATAG |
| NM_005638 | AGAAACATAGATCTGGTAGCTCAGCGAGGA |
| NM_003288 | CAGGAAACTCTTTCACAGGCAGGACAGAAG |
| NM_003302 | TCTCAGTTCCAGTTCCCATCCTTTGATTGA |
| NM_016308 | GGAAAAGAGAATTCAGACCTACCTTCAGTC |
| NM_006113 | GAGGCATTTGACTTGTTTGATGTTCGTGAC |
| NM_006802 | GAGGATCTGAAAAGACAAGGACTGCTCTAG |
| NM_004184 | GATGAGATAGTGAAAGAGTTCATGACTCCC |
| NM_000089 | CAAGGTCCAGTTGGCCGAACTGGAGAAGTA |
| NM_004951 | TTTCTAGTGCTGTGAAGTCAGCCCCTGAAG |
| NM_002600 | AGCATTCGGCAGCGTCGTCGCTTCACTGTG |
| NM_005178 | TCAGCTCCAATGGTCTTCTCTCCGCATCAC |
| NM_001753 | GCAATGTCCGCATCAACTTGCAGAAAGAAA |
| NM_002414 | CCAGCTGTTCAGCGTACTCTTTTAGAGAAA |
| NM_001788 | GAAAAACGTCGTCAGTTCGAGGATGAGAAA |
| NM_016184 | GAGCTGGTTCATACAACATTGGAGTGTGTG |
| NM_005627 | TATGCGCCTCCCACGGACTCTTTCCTCTGA |
| NM_006329 | ACTGCGGATATATGTGTCGCAGTACCCATT |
| NM_016195 | CTCTTCCTGACCCCCAGGAAGCTACTGCTT |
| NM_006310 | CTTCAGCGGAAGGCCTCACCACCCACAGTG |
| NM_003707 | GCTGACCAGCAGGATAAGTACATGAAGTGA |
| NM_001845 | CGTCAGCCGCTGCCAAGTCTGTATGAGAAG |
| NM_006803 | GCTGGCCATTTCTGGACTCAAGGTGAATCG |
| NM_001219 | CACCTACGGCTACGTTTTAGATGATCCAGA |
| NM_001260 | CAGTACTCACATCAGACACATCGGTACTGA |
| NM_001916 | ATAAAGCGGCACAAGTGGTCAGTCCTGAAG |
| NM_000121 | GCTTATCCGATGGCCCCTACTCCAACCCTT |
| NM_002394 | TTTCTGGTAGTGCTTAACTTTGGGGATGTG |
| NM_004483 | GATCAAGATGACACTGAGTAACCCTTCAGA |
| NM_000521 | GATGGTCGAACGTGGAATAGCTGCACAACC |
| XM_167711 | CTGACAAATGACAAGACCCCTGAGGCATGA |
| AL833311 | CCTGCTATCACACAGAACAACTTTCTAACC |
| NM_013995 | GTGCTGGTCTTTCAGGCTTGATTATCGTTA |
| NM_024321 | CAGAGAGCAGATTCCGCTCTCTCCTCTGCA |
| NM_005952 | TCCTGCTCGCCTGTTGGCTCCTGTGCCTGT |
| NM_005375 | CTACCAGTAAGACTGTCATCATGTGCTTGA |
| NM_000946 | CCGTGAATTGGATGCCATTTCCACTAATGA |
| NM_004176 | TGCTGCACGACTGTCAGCAGATGCTCATGC |
| NM_002958 | ACAGAGTTTCATGCAGCCCTGGGGGCCTAC |
| NM_005063 | CCAGGATTAAAAGAACCGGAGATGGAAACT |
| NM_145341 | CAGATACTGAATGTAAACCCTGCAGATCCT |
| NM_003132 | AAGTACTACAACTCCGACGTGCACCGCGCC |
| NM_005100 | CAGGAGGAGAACCGAAATATCGAACAGATT |
| NM_021822 | CCAGGACTACAGGGTTACCTGCTTCACCTC |
| NM_020993 | GCCTCTCAACAAAACTCCGAAGAGATGTAG |
| NM_015099 | GACCTGGATTGCACTTAAGTTTGCACTCTA |
| NM_005127 | CAGCAACAGCTAGATGTTACACCGAAAGAA |
| NM_004010 | CCAGCCTATTTCCTTAATGCTGTAAGGAGG |
| NM_002305 | ACCGTCAAGCTGCCAGATGGATACGAATTC |
| NM_000237 | GAAAGTGTCTCATTTGCAGAAAGGAAAGGC |
| NM_006187 | GCTGCATGGGACGGAATGGCATCCCCATCC |
| NM_005419 | GATGGACCCTTGATGCCTTCTGACTTCTAG |
| NM_002576 | CAGCTAAGGAGGCAACAAAGAACAATCACT |
| NM_002390 | ATTCGCCGAGGAAGGTCCGGAGGGGCCTAA |
| NM_016187 | GGTATCCACAAGTGAAAATCCACAACTCTG |
| NM_014207 | GACTATGATCTGCATGGGGCTCAGAGGCTG |
| NM_005214 | CCTCACAGCTGTTTCTTTGAGCAAAATGCT |
| NM_003564 | GGCTACGGGATGCCACGCCAGATCCTCTGA |
| NM_002002 | AGTCCATGGGACCTGATTCAAGACCAGACC |
| NM_004001 | ACGGCAGCGAAGCAGAGCTCCCTCGTTGGT |
| NM_058176 | GCCCTTCTAGGAAATGAGCTGGAGCCACTT |
| NM_001558 | CTCATCTCTAGCCTGCAGTCAAGTGAGTGA |
| NM_006850 | GGGAAGTGGACATTCTTCTGACCTGGATGC |
| XM_042066 | AAGCATCCAGTCTTTCGTACTACATGGTAG |
| NM_033004 | GTGGAACCTCAGGTGCAACAGAGACGCCAG |
| NM_002835 | CCAAGAGATCCACCTTCAGAATGGACATGA |
| NM_000593 | TGGAGAAAAAGGGGTGCTACTGGGCCATGG |
| NM_002927 | GGATTCCTACCCCAGATTTCTAAAGTCAGA |
| NM_005012 | GCCAATATTCATGGACACACCGAATCTATG |
| NM_000655 | CAGTGCTGTACTTGATGACAGACACTTCTA |
| NM_005449 | TCTGAATCTCCCTGGCTCCATGCCCCATCT |
| NM_133378 | GTCATGTATAACAAGTTTTCTGACCCTGCC |
| NM_032663 | CCCTCACACCAGTATTTATCCTTAACACTC |
| NM_030753 | GCCAGGAGTGTATTCGCATCTACGACGTGC |
| NM_017935 | CGACCCCAAGTTGAAAAGGAATTTGGTTTC |
| NM_003202 | ACAGGTGGCCTAGCAGGCACAGGACACCTG |
| NM_030764 | TGAATATCCCTGTGAGAATTCCAGTGTCTC |
| NM_023109 | CCATCTCAGTAGGGATCTAGCCACATCCCC |
| NM_002738 | ACCAGTCCTAACACCTCCTGACCAGGAAGT |
| NM_006472 | ACCACCGACTTATACTGAGGTGGATCCCTG |
| NM_000417 | GCCCCAGCTCATATGCACAGGTGAAATGGA |
| NM_001775 | GAGGATTCATCTTGCACATCTGAGATCTGA |
| NM_005574 | GCCTGGACCCTTCAGAGGAACCAGTGGATG |
| XM_034274 | CCAGTAGTACTTCAAGAGCTCTCATACTGT |
| NM_001706 | ACCAAGGTGCAATACCGCGTGTCAGCCACT |
| NM_000902 | AAACAGATGTGCACAGTCCAGGCAATTTCA |
| NM_001102 | ACGGCGCTGTACGGCGAGAGTGACCTCTAA |
| NM_001718 | GCGCCAACTAAGCTAAATGCCATCTCGGTT |
| NM_033554 | TCATAAAGTCTCTGCGTTCTGGCCATGACC |
| NM_002122 | CCTGTTCTCAGACAATTTAGATTTGACCCG |
| NM_019111 | CCTGTGGAACTGAGAGAGCCCAACGTCCTC |
| NM_002124 | CCACGTTTCTTGTGGCAGCTTAAGTTTGAA |
| NM_014745 | CTCACCAAGAAGTACAATCATCTCAACCTC |
| NM_006993 | ATCCTTCCTGCCAAAAAGCAGGGGGGCAGG |
| NM_014366 | TCTGAGGAGACTACAGCAGGTGAACAGTCT |
| NM_003255 | CAGGAGTTTCTCGACATCGAGGACCCATAA |
| NM_002658 | CACACTTCTTACCCTGGATCCGCAGTCACA |
| NM_002704 | GAACCCATTGCAACCAAGTCGAAGTGATAG |
| NM_000090 | TCGAGGATTCCCTGGTAATCCAGGTGCCCC |
| NM_002026 | AGGGAAATTCTTTGGAAGAAGTGGTCCATG |
| NM_001766 | AGGCAAACTTCCTATCAGGGCGTCCTGTGA |
| NM_006614 | TCTACAGCAACTTTTCCCCTTCGGGCATAA |
| NM_004445 | CCAAGTTTGGCCTCTGTACCTTCAGTGATG |
| NM_006058 | ACCAGAGCCAGCTGATCTCAGATTGCCAAG |
| NM_052938 | TCCAGGCTGAGGAAAGCAAACATTACAGAT |
| NM_004126 | AGCTTCGCAAAGAAGTGAAGTTGCAGAGAC |
| NM_007360 | GTGCACTCTATGCCTCGAGCTTTAAAGGCT |
| NM_004225 | ACCCACTGATCCAGCCCCCCTACGAGGTCT |
| NM_005940 | GGTCCTGACTTCTTTGGCTGTGCCGAGCCT |
| NM_003800 | CCTGGATGTTCTCTTATCTGGAATCCATCA |
| NM_030956 | GAGGTTCTACAATCTCTCTGATGAGAACAG |
| NM_003453 | CCTTGGAAAATATGCTTGTACGGGTTCTTC |
| NM_018136 | GGAAGAAATCACAAATCCCCTGCAAGCTAT |
| NM_000682 | ACTTCCGCCGTGCCTTCCGGAGGATCCTGT |
| NM_181802 | ATCCAGAGCCTTCTAGGAGAACCCAACATT |
| XM_058619 | CTCCACAAACTGGTTGTGCTCGGCACCTGA |
| NM_018248 | GCAAGCGTTCCACCATGAAAACAGTATTGA |
| NM_000194 | GACAAGTTTGTTGTAGGATATGCCCTTGAC |
| NM_004526 | GAGAAAGTGTCCAGCCGAGCCATCTTCACC |
| NM_002426 | CTTCCTACTCCAACGTATCACCAAAACACT |
| NM_002608 | TCTCTGCTGCTACCTGCGTCTGGTCAGCGC |
| AF108138 | TGTGCTGCACTTCTATGCCACCCTGCGGCG |
| NM_002692 | CTCTGCATAAACCCTGGCTCTTTTCCAAGA |
| NM_003349 | GTCACAAAGCAAACTGAGTGATGAAGGAAG |
| NM_001067 | GGGCAAAGAAACCTATAAAGTACCTGGAAG |
| NM_013282 | ACGTGAACAGACTCTGCCGGGTCTGCGCCT |
| NM_002356 | TCAAGAAGTCTTTCAAGCTGAGCGGCTTCT |
| NM_002592 | TACTTGGCTCCCAAGATCGAGGATGAAGAA |
| NM_003113 | GCTGCAGCTGACAAGCAGTTTTATGAAAAG |
| NM_005292 | GATCTGGTAGTCTACGGTCACTAAGCAATA |
| NM_005192 | GCAGAGTTGTCCAAACAGACACAGAAGAAC |
| NM_000022 | CCAGCAGCAGACCGTGGTAGCCATTGACCT |
| NM_005163 | CCACCTGACCAAGATGACAGCATGGAGTGT |
| NM_001295 | CCTCCACAGGGGAGCATGAACTCTCTGCTG |
| NM_006887 | ACAGCTACCTAAGCGGCTCCCTGAGCTCCG |
| NM_005191 | TTGAGAAGGGAAAGTGTACGCCCTGTATAA |
| NM_006889 | GCCCAGCGTGTTTTTAAAAGTTCGAAGACA |
| NM_001846 | ATCGAATGCAATGGAGGCCGCGGCACCTGC |
| NM_002090 | ATCGAAAAGATACTGAACAAGGGGAGCACC |
| NM_004460 | GGCCTGTCCACGAACCACTTATACACCCAC |
| NM_032682 | CTGGAAAACAGCCTAAAGAGCAACAGCAGG |
| NM_006120 | ACTCCTGGGCCGTCCCTGAAGCTCCTACTC |
| NM_002120 | CTGGTAATGAGGTCTCAAGAGCTGTTCTGC |
| NM_006010 | ACTGATGCCTAAATATGCCCCCAAGGCAGC |
| NM_001560 | GACTCTGTAGTGCTGATAGAAAACCTGAAG |
| NM_172174 | GGCATTCATGTCTTCATTTTGGGCTGTTTC |
| NM_000632 | CTTCAAGCGGCAATACAAGGACATGATGAG |
| NM_004972 | GGATCAAATAAGGGATAACATGGCTGGATG |
| NM_004995 | GCTCTACTGCCAGCGTTCCCTGCTGGACAA |
| NM_172390 | GACCTCTCCAGCACGAGCACCCACTCCTAG |
| NM_005384 | AGACTTATAGCCACACAACCAATCTCTGCT |
| NM_001675 | AAGGCAAGGGGGAAGAAAAGGGTCCCCTAG |
| NM_000960 | TCCAAAGCAGAAGCCAGCGTCGCCTGCTCC |
| NM_000963 | CCACAGTACTACTAAAAGAACGTTCGACTG |
| NM_003037 | TATGCTAGTGTGACACTTCCAGAGAGCTGA |
| NM_018664 | GCTGCTCAGAGAAGTCGGAAGAAGCAGACC |
| NM_003121 | CTCCGAAACTACGCCAAGACCGGCGAGATC |
| NM_007315 | GGCTCTGTAGAATTCGACAGTATGATGAAC |
| NM_006290 | CTACTGCAACGAATGCTTTCAGTTCAAGCA |
| NM_007115 | AATGGATCCTGTATCCAAATCCAGTCAAGG |
| NM_012452 | CAGACAGTGGCCTTGGCATTGTGTGTGTGC |
| NM_003820 | TCATTCACGGGGAGGAGCCCAAACCACTGA |
| NM_005104 | TCAGACACCAGTGATTCAGACTCAGGCTAA |
| NM_000043 | GCCAATTCCACTAATTGTTTGGGTGAAGAG |
| NM_003326 | GAATGGCGGAGAACTGATTCTTATCCATCA |
| NM_005658 | AATGTTCCTCAAGTGCATTGTGGAGACCAG |
| NM_006060 | AAGCATGGGCCTTCCGGGCACACTGTACCC |
| NM_005608 | GGAAGCCAGAGACAGTGACACGGAGGGCGA |
| NM_005348 | GTCGTGGAACAAAAGTTATCCTACACCTGA |
| NM_000033 | ACCCACCGGCCCTCCCTGTGGAAATACCAC |
| NM_004915 | TTCACTTCCCCGAGCTCCGGAGAGGGATGG |
| NM_005186 | AAGTGGTTGCAGCTGACCATGTTTGCATGA |
| NM_005166 | CGCGAACTGCAGCGGCACGGCTATGAGAAC |
| NM_015193 | GCAGCTCATCCAGAGGGGCATGGAGGTGCA |
| NM_001178 | GCAGCTCCACTGACTACCAAGAAAGCATGG |
| NM_004317 | CCATGAGGTGCGGGGGGCAGACAAGGTCAA |
| NM_001187 | GACGGCACAGCTCTGTGCTTCATCTTCTGA |
| NM_172171 | CGTGAGGCTCGGATATGTCGACTTCTGAAA |
| NM_016280 | ACCCACACCTCCTTGGCTCTATGCTCCTGA |
| NM_000743 | GAAACATCCGATCAAGAACCTGGGCTATGA |
| NM_001762 | TTCTCTTGGTTGATGAGATCATGCGAGCTG |
| NM_000085 | TTCATTCCCTCTTTGTGACGTCGCGGGGCA |
| NM_001910 | AAAACGCCATTGGGGCAGCCCCCGTGGATG |
| NM_000778 | CCTACCCACCTGTATCTCTTGTTGGGAGAA |
| U53532 | TTGTTTCGACGAGTTCAACCGGAATTCGTG |
| NM_001939 | TTCTGATGTGACTGCCAACACCCTGCTGGC |
| NM_004091 | TACGACCTTGGGGACCTGTTGATTAATTGA |
| NM_001441 | GAGTTGTGTCTGCGGTTCATGCGGGAGGTG |
| NM_000148 | TTAATGCAGACTTGTCTCCACTCTGGACAT |
| NM_001777 | AAGCTGTAGAGGAACCCCTTAATGCATTCA |
| NM_002049 | GTGCTGTCAGGGCCTGTTAGCCACCTCATG |
| NM_002068 | ACGCCTTTTCAGCCACTACACATGTGCCAC |
| NM_005275 | TGGCTGGCCGAAACCCTTATGCCCTGCTGG |
| NM_002076 | GCGGCAGTGTCAGGACTCGAAGATTTTCCA |
| NM_005288 | CCCCTCCATCTATACCTACGCCACCCTCCT |
| NM_002094 | TGCCTTGAGACCTTTAAAGACTTCCCTCAG |
| NM_005513 | TGGGACAACGCATGTTTGAGGACCTCTTTG |
| NM_004963 | GCACTCTGGAATACTTGCAGCTGAATACCA |
| NM_000182 | TAGTGGACCGGCTCAAGAAATATGAAGCTG |
| XM_033511 | CACTATGTCCCAAGATGGATTCCCAAATGA |
| NM_006825 | TGCCCTCGGCCAAACAAAGGGGCTCCAAGG |
| NM_000867 | CATTCTACTAGATACGCTTCTCCTCACTGA |
| NM_006764 | ACCAAGGCTCGAAGCCGTGTGCGGGACAAG |
| NM_000612 | CCCTGATTGCTCTACCCACCCAAGACCCCG |
| NM_006083 | AAGGGCGGAAAACCAGGCGCTTCAAGGAAA |
| NM_006084 | AGCAGGCCTTTGCCCGATACTTGCTGGAGC |
| XM_045712 | ATCGGCTCCCCAAGATTAAGGAAACCACAG |
| NM_000229 | ACCCTGTGGGTGTGCTCTATGAGGATGGTG |
| NM_000234 | TCAGAACCAACAAGGCGAGGACTCAGGCTC |
| NM_025247 | CTGGGAAAATGGAATGCAACCCACATTGTA |
| NM_004255 | GAACTGGGAATCTCCACTCCGGAGGAACTG |
| NM_133259 | CTGTCCCTTTCATTGAACCCCCTGAAAGCT |
| NM_000239 | CGTCAGTATGTTCAAGGTTGTGGAGTGTAA |
| NM_005909 | CAACATCATGGTTTTAGCAAGCAGCAGCAC |
| NM_002379 | GAAGCTGTGAGTAAGCGGCTGGCCATCCTG |
| NM_002396 | GCATCAAGCCCTCCTGTGATAACAGAATAG |
| NM_139202 | CCACTGCGGAACAGGAGTGACTCTGGCTGC |
| NM_005936 | GGTGTCATCCAGGTACGTTCCAGCCGGCCA |
| NM_002421 | GAAAGCTAATAGCTGGTTCAACTGCAGGAA |
| NM_002476 | TTCTTGCCCATCCTGCAGCACATTTCCCGC |
| NM_002523 | TCGATGTGTTCGGAGGGGCCTCCAAGTGGC |
| NM_001862 | TTGGCTGCACAAAGGCGAGGCCCAGCGATG |
| NM_006981 | CCTGCTTTCAAATGAATGATCAGGGTCTCT |
| NM_002527 | GCCTTGTCGAGAAAAATCGGAAGAACATGA |
| NM_002557 | GTAACCCCTCAAACAAGTCCTCTTTCTCTA |
| NM_014735 | GCCATGGTAAAAGCAAGACACATCCCCTTT |
| NM_032940 | TCTGCGTCCTGAAACCATTGTCCTGTCAGC |
| NM_002739 | CCAGGGCTTCACCTACGTGAACCCCGACTT |
| NM_134260 | GGAGGCAGAATGGCAAGCCACCATATTCAC |
| NM_003973 | AGCTCTCCTGAAAGCTTCTCCCAAAAAAGC |
| NM_006642 | TGAGACTGGCTGATGTTCAGAGCACTTACT |
| NM_002640 | TGGAGCCAAGATTCTGTGCAGACCACCCTT |
| NM_004374 | GGCATACGCAGATTTCTACAGAAACTACGA |
| NM_005850 | CCTATGCCTCCGCATGGTATGCGTGGACCT |
| NM_000578 | TCTGGACCTGTTGCCTTGCCCACGGAGCCA |
| NM_014251 | TATCTACCTCAAAGGCTATTGGTGGAGGCC |
| NM_006841 | CTATGCTTGGCTTCTTGCTGATGACCATGA |
| NM_033262 | GCCCCCGCATGGTGGATATGAGTTTTCAGA |
| NM_014720 | GAGGAGCTTGCACAAAGCCAGCATGCTCAG |
| NM_006938 | CAGTTTACCTCTGGATACACTACTTGTGGA |
| NM_003107 | TAACTTCGAGCCCGGCTCCGGCTCGCACTT |
| NM_012448 | AGTCAGTGGATCCCGCACGCACAATCGTGA |
| NM_006453 | TGGGAAACCCATAAAGGCGCACTGCCCTAG |
| NM_003315 | CTTTGAAGCATCTGGTCCAGGGAATTTCTT |
| NM_170695 | GTTGCACTCAAACGGGCTGCAGAGATGGAG |
| NM_005077 | ATGATAAGTACATAGTCACTGGCTCGGGGG |
| NM_133502 | GGAGGCAGAAGATTTCTGGCCAGTGGAGAG |
| NM_003430 | CTTTTAGCCATTCTTCAGCCCTTGCTAAAC |
| NM_005157 | CATGGGGGTCCACACTGCAATGTTTTTGTG |
| NM_004418 | TACCTCATGCAGAGATCGCCGTGTGCGGCTG |
| NM_001719 | AACCCGGAAACGGTGCCCAAGCCCTGCTGT |
| NM_006835 | TCCCCTTGTCCACCTTTGCAGCCTGTTTCT |
| NM_000610 | TACAAATACGGGTTCATCACTGATTCCACC |
| NM_012287 | CCCAAATGGCATCCAATAATCCAGAGAAAC |
| NM_003879 | GCCCATTGTCCTGATCTGAAAATTCTTGGA |
| NM_005197 | TGCTTTTGAATACTCCCCCTGAGATACAAG |
| NM_001882 | ATCGGGGAATTCTGTTTGTCTGGTCTTTGA |
| NM_001319 | GTGGAGGTGGCCGATGAAACCAAATGCTGC |
| NM_001921 | AACAGCAGACCGAGTCAAAAGCTTCAGTGA |
| NM_006824 | AGCAGATGAGGAAGGGGCCCAGTGCTAAAC |
| NM_003648 | ACGTCGGGTCAGATCCGACAGAAGACCATC |
| NM_004441 | GAGCCTGTATGTCTCTTCTTTCTGTCCGTG |
| NM_002025 | TGAGCAGAGCACCTTTTCCATCCCAGGACA |
| NM_001530 | ACTGCAGGGTGAAGAATTACTCAGAGCTTT |
| NM_005531 | GGGGAGTTGAGATCTGTAATTCATAGTCAC |
| NM_000874 | ACAGTCGTCCTGCCTAAGCTTCCCCAGTAG |
| NM_014002 | CGAACGGCTAAATAGAGTCCCAGCACCTCC |
| NM_000628 | ACTCTGAGAGCGGCAAGCAGAATCCTGGTG |
| NM_000880 | TGAACTGCACTGGCCAGGTTAAAGGAAGAA |
| NM_001567 | ACCCGGCTCACAAGCGCCTCCTTCTGGACA |
| NM_001398 | GACCTCGTGAAGTCGGTCCAGCCCACGACT |
| NM_005544 | CCACCCGCCGCTCAAGTGAGGATTTAAGCG |
| NM_002249 | CTCTTGGGGTTTGTACTCAAAGGACTCCAT |
| NM_002755 | CAGTGGAGTGTTCAGTCTGGAATTTCAAGA |
| NM_003954 | ACTCGGGCATCGACCTGCAGTGCACACTGG |
| NM_002748 | CCTCAAATTCCTCATCAAACATACAGCAGC |
| NM_002408 | GGGAGATATTAGGGACCATGAACTCTGTAA |
| NM_004927 | AGGAGCTTAAAGCCTGGCTCTTGGAGAAAG |
| NM_004998 | GACTACGAGGCAAGCCAGGGCTGTTCCCCA |
| NM_003908 | CGAGCACAGCTCCGTGCCAAAGCTAACTAA |
| NM_005009 | AGAGCAGTGAGCTGGTGAGCTGGGCAGACG |
| NM_002542 | GACTTCTTCCTCTAGACTTGGAGGGCCTCC |
| NM_012383 | ACTGCCTTATACTGGGCTTGCCACGGGGGC |
| NM_005746 | GATCTTCTCCATACTGTCTTCAAGAATGGC |
| NM_002649 | CATCTTGTTCTTGGCATCAAACAAGGAGAG |
| NM_021127 | GTCGAGTGTGCTACTCAACTCAGGAGATTT |
| NM_006237 | AAGCGGATGAAATTCTCTGCCACTTACTGA |
| NM_003479 | TACGCTTCAGAGATACCAATGGGCATTGCT |
| NM_014369 | GAGATAGAGAATGGGCGGAAAAGGTGTGAG |
| NM_002828 | CTTTTGTTGGCTGGAGACTGTTTTTTCAGC |
| NM_001469 | CTGCCGGGCTTACGGGCTGAAGAGTGGTCT |
| NM_001754 | GAAGCTTTCTCTAGGTGTAGCAGAAACTGC |
| NM_006918 | CTCAGTTATGTGAAGGAGATGACAGAGGGA |
| NM_004630 | CTCTCCTCACCCAACTCCCTTTGCCTCTCC |
| NM_003105 | CCAGGCTGGGGTCCGCAATCTTCTCCTCTG |
| NM_004509 | GCCTACTGTCATCCACAAAGCTCCTTTTTT |
| NM_007237 | GGTCAGTTTCTTGTTTCTCTACAGAGACCT |
| NM_003120 | ACCTACCAGTTCAGCGGCGAAGTGCTGGGC |
| NM_182692 | GGAATTCTTCAATCGCAGAGGAGAACTGCG |
| NM_001066 | GAATGTGCCTTTCGGTCACAGCTGGAGACG |
| NM_003985 | TCATCGAGGGCAGCTCCTCTTTCCACAGCC |
| NM_006708 | CTGATGATGGTAAAATGAAAGGCCTGGCAT |
| NM_003387 | CGGAGTGAGTATTTCTGCCAAGGTTTTTGA |
| NM_003422 | ATGGTCCCCTTGTGTATGCAGGGTTCGCGC |
| NM_175870 | TCAACCGTCATCCTTTCCCTCGTCCTGGCT |
| NM_006748 | TTCTCATCACCACCTTACTTTGAGGACTAG |
| NM_001877 | TCTCCTCCTGTGACTCGCTGCCCTAATCCA |
| NM_000878 | CAGGGTCAGGACCCAACTCACTTGGTGTAG |
| NM_005923 | CTAAGGGGAGGGATGCTGTGCACACTGTGG |
| NM_000941 | AGGCGGTGGACTACATCAAGAAACTGATGA |
| NM_001242 | TGCAGAGCCTTGTCGTTACAGCTGCCCCAG |
| NM_006994 | CAACAACCAATCAGAACCATAAGCTACAGG |
| NM_001629 | ACCATCTCCCCTCTACTTCTCATTCCCTAA |
| NM_002107 | CAGCTAGCACGCCGCATACGTGGAGAACGT |
| NM_021603 | TGACTGGGTTGTCGATGGACGGTGGCGGCA |
| NM_003376 | TCTGGGCTGTTCTCGCTTCGGAGGAGCCGT |
| NM_000034 | TCAAATCCAAGGGCGGTGTTGTGGGCATCA |
| NM_004044 | TGGCTCCCGGCCAGCTCGCCTTATTTAGTG |
| NM_005998 | AGAAAGGCGATGACCAGAGCCGGCAAGGCG |
| NM_002156 | GGTGCAATGGGTGGAATGGGAGGTGGTATG |
| NM_002168 | CCGTATTATCTGGCAGTTCATCAAGGAGAA |
| NM_000884 | CTCCATTCGTATGAGAAGCGGCTTTTCTGA |
| NM_005566 | TGGGGGATCCAAAAGGAGCTGCAATTTTAA |
| NM_006636 | GTCTAAAGAGCTTGGGGTAGCCACTAATTA |
| NM_000269 | CTGGTAGATTACACGAGCTGTGCTCAGAAC |
| NM_002629 | GGATGAAGAGACGGTGCGCAAAGCCATGGA |
| NM_000365 | CTCAAGCCCGAATTCGTGGACATCATCAAT |
| NM_001148 | GAGGACATCTTTGACACAAGGCCCATTTGG |
| NM_138271 | GAAAATCTCAAAAAACGCGGAGAAGATGGG |
| NM_004049 | ACACAGGAGAATGGATAAGGCAAAACGGAG |
| NM_006624 | CACAAGCAACTGATTTCTCAGACCAAGAAG |
| NM_004166 | TCCGGGAGTTCAGGATTGCATGAAAAAGCT |
| NM_002989 | AGGACTGAGCGGTCACAGACCCCTAAAGGG |
| NM_003542 | TAAAACGTCAGGGGCGCACTCTGTATGGCT |
| NM_012073 | GATTGATGACATTCGTAAGCCTGGAGAATC |
| NM_000732 | CCCCTCCGAGATCGAGATGATGCTCAGTAC |
| NM_000733 | TATTCTGGCCTGAATCAGAGACGCATCTGA |
| NM_001781 | ACAACTGGTTCAACGTTACAGGGTCTGACA |
| NM_001809 | CCAGGCCCTATTGGCCCTACAAGAGGCAGC |
| NM_001280 | AGACAGTTACGACAGTTACGCTACACACAA |
| NM_007096 | CCATCCTTGCTACAGCCTAGAACAGGCAGC |
| NM_001891 | CCACCCACCAGATCTACCCTGTGACTCAGC |
| NM_001321 | GGAGCAGGGGCTCTTGTTCATGCCCAGTAA |
| NM_001908 | GAATTCCACGCACCGATCAGTACTGGGAAA |
| NM_005517 | AACCACAGAGAAGATCCGCGAGGTTGTCTG |
| NM_001909 | TCTTCATCGGCCGCTACTACACTGTGTTTG |
| NM_014750 | GGAGACATCAAGAACATGCCAGACACATTT |
| NM_004944 | CCTTCACCAACAGCAAAAAATCTGTCACTC |
| NM_004089 | AGAGCCAGCTCCCGAATCCCCACAAGTGCC |
| NM_004111 | GGCAGCAGGGAAGTTTAAAAGGGGAAAATA |
| NM_002036 | TCTGGGTATGTCCTCCAGGCGGAGCTCTCC |
| NM_006851 | CCACGTAACAGATACACTTCTCTCTTTCTC |
| NM_000405 | AAGATCGCTGCCTCTCTAAAGGGCATATAG |
| NM_002110 | GGAGCGGCCGACCTTCGAATACATCCAGAG |
| NM_005524 | TCCTCATTCCCAACGGGGCCTTCGCGCACA |
| NM_002136 | CGGCTTTGGCGGTGGTAGTGGAAGCAATTT |
| NM_145904 | GAGGGGTGAGTCCCCTACTCCCTCTTCACT |
| NM_005527 | CTGCGGAACAGGGTATGTGCCTGGAAGGCC |
| NM_007355 | GATGCGTCTCGCATGGAAGAAGTCGATTAG |
| NM_004258 | GTGACCACAAATAGGAGGGAAGACGAGGAG |
| NM_002194 | CTGGTCCAAAACCTGGCACCTGCAGAGACG |
| NM_000885 | TCTCATATGTTATGTGGAAGGCTGGCTTCT |
| NM_002306 | GACCTCACCAGTGCTTCATATACCATGATA |
| NM_002348 | CTATACTCTGTGCTCTCCCAAGGATATGAG |
| NM_005916 | CTAGGAGACAAGGGGCAGACAGCTAGGACT |
| NM_005520 | TGTCAAACCAGTCCAGCTACGGGGGCCCAG |
| NM_002397 | TGTCTGAGGATGTCGACCTGCTTTTGAATC |
| NM_014791 | GGAAGACATCCTATCTAGCTGCAAGGTATA |
| NM_000918 | GCAGGGGATGATGACGATCTCGAGGACCTG |
| NM_002627 | TCAGGCCAGCTGGAACATGTGCAGCCCTGG |
| NM_006219 | AGGGAAAGCTGGACTACTAAAGTGAACTGG |
| NM_002654 | AACACCATGCGTGTTGTTCCTGTGCCGTGA |
| NM_000302 | TACATCGCAGTCTCCTTCGTCGATCCCTAA |
| NM_002674 | AGAGTCTACCGACCCTGTTGGCAAGTCTGA |
| NM_002702 | GCAATCGGCGCCAGACGCTCAAGAACACCA |
| NM_002802 | GAAGGCACCCCTGAGGGGCTGTATCTCTAA |
| NM_005055 | ATGACCGAGATCGGAAACCGCCTGGGGCAG |
| NM_015004 | GCCTCCTTGCAGAGTGTTCTGCACAAGGAA |
| NM_002970 | ATTTTACCTATGACCCGTGGATTGGCAAGT |
| NM_005410 | AAGCCAGTGCCAGTTGACGCTGAAAGAATC |
| NM_005411 | ATGGAGTGAGTCAGTGACAGGAGCAGCGAC |
| NM_003034 | GAAGATACCTCACTCCAGCCCACTTCCTAG |
| NM_005628 | AACCCCCTCCTCAAACACTATCGGGGGCCC |
| NM_004955 | AGAGACCGCAGGAGCCATCATGGCCTTCTT |
| NM_031844 | AGGTAAGAAGAAGGCGGAAGGCGGCGGAGG |
| NM_003091 | GATTGTCTGGTTTCCTTACAGGGCCCCCTC |
| NM_004780 | GCAAAACGGGGCGGTCCTTATCCTATTTAA |
| NM_005650 | GCAGAACAAGACCGCGAAAGGCAGCCTCAG |
| NM_003249 | TGCATCCTGAGACCCGGCGGTTCCGAGGAT |
| NM_000074 | ATGGCACTGGCTTCACGTCCTTTGGCTTAC |
| NM_016292 | AGGGCCATGGTGGGCCGCTTGAATGAGCTG |
| NM_021643 | GGAAGAGAACTTGGACCCTTTCTTTAACTG |
| BX436497 | AACCCAGCTCTCTGTCTTGGAGGACCTGAA |
| NM_006088 | ACAATGTGAAAACGGCTGTCTGTGACATCC |
| NM_005347 | ATGAAGCTCTCCCTGGTGGCCGCGATGCTG |
| NM_003328 | CTGGCACCAATGTCCATATATGAAGTCATG |
| NM_007013 | CATGAAAATGGAGAGCCTTCAGCAAGGACA |
| NM_003441 | AGACGGTCCACAGATCGGAGTCAACATAAG |
| NM_021089 | GGGTGCTTCCATGTTATTTGACATCAGAGA |
| NM_004310 | AGGAGACGAAACAGAAGGAGGCTCTTCTCC |
| NM_016733 | GGCACGGCTCTTGCTTCCGGTGTTCAGAAT |
| NM_002818 | GAGAAAATTGTCACCCCAAAGGGTGAAGAA |
| NM_005018 | TTCCTAGCGGAATGGGCACCTCATCCCCCG |
| NM_002574 | GACAAACATGGGGAAGTGTGCCCAGCTGGC |
| NM_031966 | TAGTTCAAGATTTAGCCAAGGCTGTGGCAA |
| NM_006854 | GTAGTCCAGACCATCCTATACTGTGACTTC |
| NM_006332 | TCCAGACCCTCGGCACCTGCTACTTACCAA |
| NM_004619 | AGATGACACTCTGTTCTTGAAAGTGGCCGT |
| NM_016155 | GGTCTGCTCATGCACCTCTGGGGCATCCTC |
| NM_002659 | TGTGAGGAAGCCCAAGCTACTCATGTATAA |
| NM_000485 | TGGCACCTGTACCCTTCTTCTCTCTCCTGC |
| NM_006763 | AAGAACTACGTGATGGCAGTCTCCAGCTAG |
| NM_001743 | ATCGATGGAGATGGCCAGGTCAATTATGAA |
| NM_006191 | GAGAATCCCACCAGTGGGGAAACATTAGAA |
| NM_176783 | CAGGGGAGAAACAAAGGGAATGATCTATTG |
| NM_002961 | CTTTGAAGGCTTCCCAGATAAGCAGCCCAG |

The present invention further contemplates arrays that comprise an HCP combination as a subset of a larger collection of probes for applications other than profiling hematological cancers.

As indicated above, in addition to the polynucleotide probes of the HCP combination, the arrays can comprise one or more control probes. Controls that can be included on the arrays of this invention include hybridization controls, scanning controls, normalization controls, expression level controls and mismatch controls. The controls can be positive or negative controls, and may be selected to target bacterial sequences that are not found in humans. Examples of positive control target genes that can be included on arrays are found in Table 29 below. Examples of negative control target genes that can be included on arrays are found in Table 30 below.

TABLE 29
Exemplary positive control target genes

| Target Sequences | |
|---|---|
| (GenBank ™ | |
| Accession No.) | Description |
| NM_000518 | Human gene sequence (Homo sapiens hemoglobin, beta (HBB), mRNA) |
| NM_000518 | Human gene sequence (Homo sapiens hemoglobin, beta (HBB), mRNA) |
| NM_000518 | Human gene sequence (Homo sapiens hemoglobin, beta (HBB), mRNA) |
| NM_000518 | Human gene sequence (Homo sapiens hemoglobin, beta (HBB), mRNA) |
| NM_000518 | Human gene sequence (Homo sapiens hemoglobin, beta (HBB), mRNA) |
| CP000001 | Bacterial sequence (Bacillus cereus E33L, complete genome) |
| CP000001 | Bacterial sequence (Bacillus cereus E33L, complete genome) |
| CP000001 | Bacterial sequence (Bacillus cereus E33L, complete genome) |
| CP000001 | Bacterial sequence (Bacillus cereus E33L, complete genome) |
| CP000001 | Bacterial sequence (Bacillus cereus E33L, complete genome) |
| NC_007384 | Bacterial sequence (lla sonnei Ss046, complete genome) |
| NC_007384 | Bacterial sequence (lla sonnei Ss046, complete genome) |
| NC_007384 | Bacterial sequence (lla sonnei Ss046, complete genome) |
| NC_007384 | Bacterial sequence (lla sonnei Ss046, complete genome) |
| NC_007384 | Bacterial sequence (lla sonnei Ss046, complete genome) |
| AK152894 | Mouse sequence (Mus musculus activated spleen cDNA, RIKEN full-length |
| | enriched library, clone: F830213J22 product: polynucleotide kinase 3′- |
| | phosphatase, full insert sequence) |
| AK152894 | Mouse sequence (Mus musculus activated spleen cDNA, RIKEN full-length |
| | enriched library, clone: F830213J22 product: polynucleotide kinase 3′- |
| | phosphatase, full insert sequence) |
| AK152894 | Mouse sequence (Mus musculus activated spleen cDNA, RIKEN full-length |
| | enriched library, clone: F830213J22 product: polynucleotide kinase 3′- |
| | phosphatase, full insert sequence) |
| AK152894 | Mouse sequence (Mus musculus activated spleen cDNA, RIKEN full-length |
| | enriched library, clone: F830213J22 product: polynucleotide kinase 3′- |
| | phosphatase, full insert sequence) |
| AK152894 | Mouse sequence (Mus musculus activated spleen cDNA, RIKEN full-length |
| | enriched library, clone: F830213J22 product: polynucleotide kinase 3′- |
| | phosphatase, full insert sequence) |
| AK172558 | Mouse sequence (Mus musculus activated spleen cDNA, RIKEN full-length |
| | enriched library, clone: F830213J22 product: polynucleotide kinase 3′- |
| | phosphatase, full insert sequence) |
| AK172558 | Mouse sequence (Mus musculus activated spleen cDNA, RIKEN full-length |
| | enriched library, clone: F830213J22 product: polynucleotide kinase 3′- |
| | phosphatase, full insert sequence) |
| AK172558 | Mouse sequence (Mus musculus activated spleen cDNA, RIKEN full-length |
| | enriched library, clone: F830213J22 product: polynucleotide kinase 3′- |
| | phosphatase, full insert sequence) |
| AK172558 | Mouse sequence (Mus musculus activated spleen cDNA, RIKEN full-length |
| | enriched library, clone: F830213J22 product: polynucleotide kinase 3′- |
| | phosphatase, full insert sequence) |
| AK172558 | Mouse sequence (Mus musculus activated spleen cDNA, RIKEN full-length |
| | enriched library, clone: F830213J22 product: polynucleotide kinase 3′- |
| | phosphatase, full insert sequence) |
| NC_007292 | Bacterial sequence (Candidatus Blochmannia pennsylvanicus str. BPEN, |
| | complete genome) |
| NC_007292 | Bacterial sequence (Candidatus Blochmannia pennsylvanicus str. BPEN, |
| | complete genome) |
| NC_007292 | Bacterial sequence (Candidatus Blochmannia pennsylvanicus str. BPEN, |
| | complete genome) |
| NC_007292 | Bacterial sequence (Candidatus Blochmannia pennsylvanicus str. BPEN, |
| | complete genome) |
| NC_007292 | Bacterial sequence (Candidatus Blochmannia pennsylvanicus str. BPEN, |
| | complete genome) |

TABLE 30
Exemplary negative control target genes

| Target | |
|---|---|
| Sequences | |
| (GenBank ™ | |
| Accession No.) | DeSCRI10ption |
| NC_007292 | Bacterial sequence (Candidatus Blochmannia pennsylvanicus str. BPEN, complete |
| | genome) |
| NC_007292 | Bacterial sequence (Candidatus Blochmannia pennsylvanicus str. BPEN, complete |
| | genome) |
| NC_007292 | Bacterial sequence (Candidatus Blochmannia pennsylvanicus str. BPEN, complete |
| | genome) |
| NC_007292 | Bacterial sequence (Candidatus Blochmannia pennsylvanicus str. BPEN, complete |
| | genome) |
| NC_007292 | Bacterial sequence (Candidatus Blochmannia pennsylvanicus str. BPEN, complete |
| | genome) |
| NC_007384 | Bacterial sequence (Shigella sonnei Ss046, complete genome) |
| NC_007384 | Bacterial sequence (Shigella sonnei Ss046, complete genome) |
| NC_007384 | Bacterial sequence (Shigella sonnei Ss046, complete genome) |
| NC_007384 | Bacterial sequence (Shigella sonnei Ss046, complete genome) |
| NC_007384 | Bacterial sequence (Shigella sonnei Ss046, complete genome) |
| NC_007384 | Bacterial sequence (Shigella sonnei Ss046, complete genome) |
| NC_007384 | Bacterial sequence (Shigella sonnei Ss046, complete genome) |
| NC_007384 | Bacterial sequence (Shigella sonnei Ss046, complete genome) |
| NC_007384 | Bacterial sequence (Shigella sonnei Ss046, complete genome) |
| NC_007384 | Bacterial sequence (Shigella sonnei Ss046, complete genome) |
| NC_007384 | Bacterial sequence (Shigella sonnei Ss046, complete genome) |
| NC_007384 | Bacterial sequence (Shigella sonnei Ss046, complete genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| NC_004547 | Bacterial sequence (Erwinia carotovora subsp. atroseptica SCRI1043, complete |
| | genome) |
| | Bacterial sequence (E. coli rafA, rafB, and rafD genes encoding alpha-D- |
| M27273 | galactosidase, raf-permease, and raf-invertase, complete cds) |
| | Bacterial sequence (E. coli rafA, rafB, and rafD genes encoding alpha-D- |
| M27273 | galactosidase, raf-permease, and raf-invertase, complete cds) |
| M27273 | Bacterial sequence (E. coli rafA, rafB, and rafD genes encoding alpha-D- |
| | galactosidase, raf-permease, and raf-invertase, complete cds) |

An example of a hybridization control would be to include several spots of the same polynucleotide probe on one chip, each spot containing a different amount of probe. This allows for the amount of probe of a given sequence to be optimized. Another example of a hybridization control is dimethyl sulfoxide (DMSO), which is used as a negative control (i.e. should give no signal), and can also be used as a scanning control. Plant or bacterial sequences having sufficiently low homology with human sequences can also be utilized as negative hybridization and scanning controls. If a signal is detected at a plant spot, this could indicate a problem with hybridization, i.e. too low a hybridization stringency was used, or with scanning, for example, the chip was inserted into the scanner at the incorrect orientation. Poly A can be used as a positive hybridization specificity/non specificity control. A poly A spot should always give intense hybridization, thus if no signal is detected at a poly A spot this could indicate use of too high a hybridization stringency.

Dyes, for example fluorescent dyes such as Cy3 or Cy5, can be incorporated into a nucleic acid sequence, such as a PCR product, and can be used as a positive scanning control for chip arrays that utilise these fluorescent dyes. Other detectable labels such as gold, for example colloidal gold or other particles, can also be used. A positive scanning control should always give a signal.

Normalization controls are nucleic acid probes that are complementary to labeled reference oligonucleotides or other nucleic acid sequences that are added to the test sample. The signals obtained from the normalization controls after hybridization provide a control for variations in hybridization conditions, label intensity, “reading” efficiency and other factors that may cause the signal of a perfect hybridization to vary between arrays. For example, signals (such as fluorescence intensity) read from all other probes in the array can be divided by the signal from the control probe(s) thereby normalizing the measurements.

Expression level controls are probes that hybridize specifically with constitutively expressed genes in the test sample. A variety of constitutively expressed genes known in the art can be used for expression level controls, for example, constitutively expressed “housekeeping genes” such as the β-actin gene, the transferrin receptor gene, the GAPDH gene, and the like. Mismatch controls may also be provided for the probes to the target genes, for expression level controls or for normalization controls. Mismatch controls are oligonucleotide probes or other nucleic acid probes identical to their corresponding test or control probes except for the presence of one or more mismatched base that are sufficient to prevent hybridization. Controls for arrays can be synthesized by standard techniques or purchased from a variety of commercial suppliers, for example, Stratagene (SpotReport™, La Jolla, Calif., USA). Other controls suitable for inclusion in arrays are known in the art.

The array may additionally comprise one or more probes each of which is targeted to a gene that is diagnostic of a hematological cancer on a general level, but which does not provide any information regarding the type/subtype, or other features of a hematological cancer. Examples of such these genes that relevant to lymphoma are listed in Table 31.

TABLE 31
Genes that show common expression patterns in all
subtypes of lymphoma

| Symbol | Modulation |
|---|---|
| 11-Sep | Over |
| ABCC4 | Under |
| ABCD1 | Under |
| ADRB2 | Under |
| AKR1C3 | Under |
| ANXA1 | Under |
| ARRB2 | Under |
| ASPM | Over |
| BIRC5 | Over |
| C15orf20 | Over |
| CAT | Under |
| CCL5 | Under |
| CCNA2 | Over |
| CCNB1 | Over |
| CCR1 | Under |
| CD33 | Under |
| CD36 | Under |
| CDC2 | Over |
| CDC20 | Over |
| CDCA1 | Over |
| CDH5 | Over |
| CDKN3 | Over |
| CENPF | Over |
| CES1 | Under |
| CHEK1 | Over |
| CHN2 | Under |
| CIAS1 | Under |
| CLECSF6 | Under |
| CNN3 | Over |
| COL1A2 | Over |
| CSF3R | Under |
| CSPG2 | Under |
| CTGF | Over |
| CTSB | Under |
| CX3CR1 | Under |
| DAF | Under |
| DF | Under |
| DGKA | Under |
| DHFR | Over |
| DNAJB6 | Under |
| DOC1 | Over |
| DOK2 | Under |
| DPYD | Under |
| ENPP2 | Over |
| EVI2B | Under |
| FBN2 | Under |
| FEN1 | Over |
| FGR | Under |
| FLI1 | Under |
| FLNA | Under |
| FLPRL1 | Under |
| FN1 | Over |
| FOS | Under |
| GBA2 | Under |
| GEM | Over |
| GNS | Under |
| HAL | Under |
| HCK | Under |
| HHEX | Under |
| HIST1H2AC | Under |
| HOXA4 | Over |
| HOXA5 | Over |
| ID2 | Under |
| IGFBP7 | Over |
| IL2RA | Over |
| KIAA0247 | Under |
| KIF2C | Over |
| KLF4 | Under |
| LILRB3 | Under |
| MBNL1 | Under |
| MCM2 | Over |
| MCM5 | Over |
| MGC5395 | Under |
| MLC1SA | Over |
| MMP9 | Over |
| MSN | Under |
| MYO1B | Over |
| NRIP1 | Under |
| P2RY5 | Under |
| PCAF | Under |
| PCDH9 | Under |
| PF4 | Under |
| PFC | Under |
| PLAU | Over |
| POLD2 | Under |
| PPBP | Under |
| PTGDR | Under |
| PTPN12 | Under |
| RAB13 | Over |
| RELB | Over |
| RGL1 | Over |
| RNASE1 | Over |
| RNASE2 | Under |
| RPS6KA1 | Under |
| S100A12 | Under |
| S100A4 | Under |
| S100A6 | Under |
| SAMHD1 | Under |
| SATB1 | Under |
| SELPLG | Under |
| SERPING1 | Over |
| SESN3 | Over |
| SFRS3 | Over |
| SLC11A1 | Under |
| SMTN | Over |
| SNX2 | Under |
| SPIB | Over |
| SYNPO | Over |
| TBC1D8 | Under |
| TCF4 | Over |
| TDO2 | Over |
| TIE1 | Over |
| TM4SF2 | Over |
| TRIP6 | Over |
| TXNIP | Under |
| VIPR1 | Under |
| WEE1 | Over |

### 2.1 Preparation of the HCP Array

The HCP arrays can be prepared using one or a combination of standard array synthesis methods known in the art, such as spotting technology or solid phase synthesis.

For example, the HCP array can be synthesized either in situ (see, for example, Hughes, T R, et al. Nature Biotechnology, 19:343-347 (2001)) or by conventional synthesis followed by immobilization onto the substrate, for example using robotic spotting. Other in situ synthesis or depositing technologies currently being used to manufacture oligonucleotide-based chips and could be used to prepare the HCP arrays of the present invention (see for example, Lockhart D J, et al., Nat. Biotechnol. 1996 December; 14(13):1675-80 and Yershov G, et al., Proc Natl Acad Sci USA. 1996 May 14; 93(10):4913-8). Many kits and packages for preparing arrays are commercially available, for example, the Pronto!™ Microarray Printing kit (Promega, Madison, Wis.). In addition, many companies provide custom array synthesis services.

Detailed discussion of methods for attaching nucleic acids to a solid substrate can be found in, for example, U.S. Pat. Nos. 5,837,832; 5,215,882; 5,707,807; 5,807,522; 5,958,342; 5,994,076; 6,004,755; 6,048,695; 6,060,240; 6,090,556 and 6,040,138.

### 2.2 Testing of HCP Arrays

HCP arrays can be tested for their ability to detect the expression pattern of genes in the one or more HCP sets that they represent using methods known in the art and one or more appropriate biological samples. Appropriate biological samples include those listed above in section 1.3.1. For example, blood or tissue samples from patients with a hematological cancer, or samples from cultures of cell lines as described in section 1.3.1, can be used to prepare labelled RNA samples. These labelled RNA samples can be hybridized to the HCP array to determine the expression pattern of the genes targeted by the HCP combination on the array according to methods known in the art.

### 3. Methods of Profiling Hematological Cancers

The system of the present invention further provides for methods of profiling hematological cancers using the HCP combinations or HCP arrays described above. These methods provide for the determination of the expression pattern of genes of one or more HCP sets in a test sample taken from a subject having, suspected of having, or suspected of being at risk of developing, a hematological cancer. In general such methods involve contacting a test sample with an HCP combination or HCP array under conditions that permit hybridization of the probe(s) in the combination or array to any target nucleic acid(s) present in the test sample and then detecting any probe:target duplexes formed as an indication of the presence of the target nucleic acid in the sample. Expression patterns thus determined can be compared to one or more reference expression patterns to provide information relating to the features of the hematological cancer(s) under investigation.

A test sample can be contacted with each probe in an HCP combination sequentially or simultaneously. Contacting a test sample with an HCP array, for example, allows for the simultaneous analysis of several features of one or more types of hematological cancers. In one embodiment of the present invention, the methods employ an HCP array. In another embodiment, the methods allow for the screening of several features of one or more types of hematological cancers in a single assay.

### 3.1 Test Samples

Test samples suitable for use in the methods of the present invention comprise nucleic acids that provide gene expression information (i.e. comprise mRNA, or nucleic acids derived from mRNA). The test sample can be, for example, a blood or biopsy sample taken from the subject to be profiled. A biopsy sample can comprise, for example, cells or tissue from a hematological cancer.

The test sample can be a biological sample that is used directly in a method of the invention, or it can be a biological sample that is submitted to one or more preparation steps, for example, the test sample may be submitted to an enrichment or culture step to increase the number of cells in the sample; to one or more extraction, purification, and/or amplification steps to isolate, purify and/or amplify nucleic acids in the sample, to one or more reverse transcription or transcription steps, or combinations of these procedures and thereby provide a test sample for use in the methods of the invention.

For example, if required, nucleic acids for use in the methods of the present invention can be isolated from the sample according to one or a combination of a number of methods well known to those of skill in the art (see, for example, Laboratory Techniques in Biochemistry and Molecular Biology: Hybridization With Nucleic Acid Probes, Part I. Theory and Nucleic Acid Preparation, P. Tijssen, ed. Elsevier, New York, N.Y. (1993)). For example, cDNA corresponding to the mRNA in the test sample can be prepared by reverse transcription of the mRNA, and the resulting cDNA can be utilised in the methods of the invention. Alternatively, RNA can be transcribed from the cDNA using in vitro transcription techniques to provide cRNA, or DNA can be amplified from the cDNA using standard amplification methodology, such as PCR. The resulting cRNA or amplified DNA can then be used in the hematological cancer profiling methods. In the above cases, the isolated nucleic acids provide a test sample suitable for used in the methods of the present invention.

The test sample can also include control nucleic acids. For example, to ensure that any amplification and/or labeling procedures do not change the true distribution of target nucleic acids in a sample. For this purpose, a sample can be spiked with a known amount of a control polynucleotide and a control probe that specifically hybridizes to the control polynucleotides. After hybridization and processing, the hybridization signals obtained from the hybridised control polynucleotide and probe should reflect accurately the amount of control target polynucleotide added to the sample. Other controls are known in the art and can be included in the test sample.

Prior to using the test sample in the profiling method, it may be desirable to fragment the nucleic acids in the test sample. Fragmentation can improve hybridization by minimizing secondary structure and cross-hybridization to other nucleic acids in the test sample or to non-complementary polynucleotide probes. Fragmentation can be performed by mechanical or chemical means known in the art.

The nucleic acids in the test sample can be labeled if necessary with one or more detectable labels to allow for detection of hybridized probe/target duplexes. A detectable label is a moiety that can be detected by spectroscopic, photochemical, biochemical, bioelectronic, immunochemical, electrical, optical, chemical means and the like. Examples include, but are not limited to, radioisotopes, chemiluminescent compounds, labeled binding proteins, heavy metal atoms, spectroscopic markers (such as fluorescent markers and dyes), magnetic labels, linked enzymes, mass spectrometry tags, spin labels, electron transfer donors and acceptors, colloidal particles, and the like. Exemplary dyes include quinoline dyes, triarylmethane dyes, phthaleins, azo dyes, cyanine dyes and the like. Non-limiting examples of fluorescent markers include fluorescein, phycoerytirin, rhodamine, lissamine, Cy3 and Cy5. Biotin and colloidal gold are also suitable for labeling the nucleic acids in the test sample. Methods of labeling nucleic acids are well known in the art.

### 3.2 Hybridization and Detection

The hybridization and detection steps of the methods of the present invention can be carried out using standard techniques known in the art. In general during the hybridization step, the test sample is contacted with an HCP combination under conditions that permit hybridization of the probes in the combination to their target sequences. Hybridization can be, for example, standard solution hybridization, utilizing an HCP combination of probes in solution, or array hybridization utilizing an HCP array.

Examples of solution hybridization techniques contemplated by the present invention include, but are not limited to, Northern analysis, clone hybridization, cDNA fragment fingerprinting, subtractive hybridization, differential display, differential screening and combinations of these techniques (see, for example, Lockhart and Winzeler (2000) Nature 405:827-836, and references cited therein). Such methods are well known in the art.

In solution hybridization techniques the probes can be labeled with a detectable label, for example, a radioisotope, fluorophore, chemiluminophore, enzyme, colloidal particle, and fluorescent microparticle, antigen, antibody, hapten, avidin/streptavidin, biotin, enzyme cofactor/substrate, and the like. Alternatively, the presence of a probe:target hybrid can be detected using an intercalating dye such as ethidium bromide, SybrGreen, SybrGold, and the like.

Other techniques include those based on the polymerase chain reaction (PCR) and other amplification technologies, such as in vitro transcription (IVT), NASBA and other isothermal amplification techniques. Such methods typically involve reverse transcriptase-PCR (RT-PCR), in which cDNA is prepared from the test sample mRNA by reverse transcription using a poly-dT oligonucleotide primer, and the cDNA is then subjected to PCR. The PCR product is then detected using probes of the HCP combination. As indicated above, the probes can be labeled or unlabeled probes can be used in combination with an interchelating dye. If desired, the PCR product can be evaluated in real-time using methods known in the art, for example using techniques involving fluorescence resonance energy transfer (FRET), in which case, the probes could be TaqMan® probes, molecular beacon probes, Scorpion probes, or the like. Technology platforms suitable for analysis of PCR products include the ABI 7700, 5700, or 7000 Sequence Detection Systems (Applied Biosystems, Foster City, Calif.), the MJ Research Opticon (MJ Research, Waltham, Mass.), the Roche Light Cycler (Roche Diagnostics, Indianapolis, Ind.), the Stratagene MX4000 (Stratagene, La Jolla, Calif.), and the Bio-Rad iCycler (Bio-Rad Laboratories, Hercules, Calif.). Molecular beacons and other sensitive probes can also be used to detect the presence of a nucleic acid sequence in a mRNA or cDNA sample that has not been submitted to an amplification step.

Alternatively, the hybridization and detection steps can involve standard array-based techniques in which an HCP array of the invention is employed. Such array-based techniques generally involve contacting a test sample with the array under hybridization conditions, whereby complexes are formed between the polynucleotide probes on the HCP array and any complementary target nucleic acid molecules in the test sample. The presence of probe:target complexes is then detected, either qualitatively or quantitatively. Specific hybridization technology which may be practiced to generate the expression patterns are well known in the art, and include the technology described in U.S. Pat. Nos. 5,324,633; 5,470,710; 5,492,806; 5,525,464; 5,800,992; as well as WO 95/21265; WO 96/31622; WO 97/10365; WO 97/27317; and EP 373 203.

### 3.3 Determination of the Expression Pattern

The level of gene expression of one or more target genes in the test sample being evaluated, or the “expression pattern,” can be evaluated by qualitative and/or quantitative measures. Qualitative techniques detect differences in expression and classify these differences in expression into distinct modes without providing significant information regarding quantitative aspects of expression. For example, a technique can be described as a qualitative technique if it detects the presence or absence of expression of a gene sequence, i.e. an on/off pattern of expression. Quantitative techniques rate expression on a scale, for example, a scale of 0-5, a scale of 1-10, a scale of +−+++, from grade 1 to grade 5, a grade from a to z, or the like. It will be understood that the numerical, and symbolic examples provided are arbitrary, and that various graduated scales (or symbolic representations of graduated scales) can be employed in the context of the present invention to describe quantitative differences in gene expression. Typically, such techniques yield information corresponding to a relative increase or decrease in expression with respect to an appropriate control.

Various techniques known in the art that yield either quantitative or qualitative expression data are suitable for evaluating gene expression in a test sample in the methods of the present invention. In some cases, for example, when multiple techniques are employed to determine expression patterns of target genes, the determined expression pattern may be the result of a combination of quantitative and qualitative data.

### 3.4 Data Analysis

Once the expression pattern for the test sample has been determined, it can be compared with one or more reference or control expression patterns using methods known in the art. The increase or decrease in the level of expression of the various genes being interrogated with the HCP combination provides information relating to one or more features of a hematological cancer in the subject from which the test sample was taken. A reference or control expression pattern in the context of the present invention is an expression pattern obtained from probing a sample taken from an individual known to exhibit a particular feature of the hematological cancer under investigation (a positive reference or control expression pattern) or a sample taken from an individual known to lack a specific feature of the hematological cancer (a negative reference/control pattern). Alternatively, the reference may be the expression pattern obtained from probing a test sample taken from the same individual at a different time point, for example at an earlier stage in the disease, or prior to a particular therapy or treatment, and can thus act as a reference for whether the disease has progressed in that individual and/or to investigate the effects of a certain treatment. It will be readily apparent that when multiple features of a specific hematological cancer are being investigated that an expression pattern determined using the methods of the invention may need to be compared to several references in order to obtain information relating to all features under investigation. The methods of the present invention are also suitable for providing reference or control expression patterns.

In certain embodiments, the expression pattern determined from the test nucleic acid sample is compared to a single reference/control pattern to determine a feature of a hematological cancer. In other embodiments, the expression pattern determined from the test nucleic acid sample is compared to two or more different reference/control patterns to determine features of a hematological cancer. For example, the expression pattern determined from the test sample may be compared to a positive and negative reference pattern to obtain confirmed information regarding a feature of a hematological cancer.

The comparison of the expression pattern determined from the test sample and the one or more reference/control patterns can be performed using a number of methodologies known in the art, for example, by comparing digital images of the expression patterns, by comparing databases of expression data, etc. Patents describing ways of comparing expression patterns include, but are not limited to, U.S. Pat. Nos. 6,308,170 and 6,228,575.

The comparison step results in information regarding how similar or dissimilar the obtained expression pattern is to the control/reference patterns, which similarity/dissimilarity information is employed to determine the feature of the hematological cancer. For example, similarity with a positive control indicates that the test sample has the feature(s) of the hematological cancer present in the control sample. Likewise, dissimilarity with a positive control indicates that the test sample lacks these feature(s) of the hematological cancer.

Depending on the type and nature of the reference/control pattern(s) to which the expression pattern determined from the test sample is compared, the above comparison step yields a variety of different types of information regarding the test sample that is assayed. As such, the above comparison step can yield information regarding multiple features of hematological cancers.

It is contemplated that one or more databases of reference/control expression patterns can be compiled and that the expression pattern of a test sample can be carried out using a computer program that compares the expression pattern of the test sample with those in the database. This type of analysis would allow information regarding the features of the hematological cancer under investigation to be obtained rapidly and would further provide for rapid throughput of multiple test samples.

### 3.5 HCP Signatures

As noted above, the methods and system of the present invention can be used to determine the expression pattern of genes from one or more HCP sets by probing a test sample with an HCP combination. Once a representative number of test samples have been probed, the expression pattern obtained from each test sample for the genes from the one or more HCP sets can be analysed in order to identify “HCP signature genes” that represent one or more features of a hematological cancer. HCP signature genes are identified on the basis of the extent that the level of expression of the gene changes when compared to the control. For example, statistical analysis can be employed in order to determine which genes show a statistically significant change in expression level over the control. Alternatively, a pre-determined differential expression limit can be imposed as a “cut-off” value. In one embodiment, HCP signature genes are defined as genes that show at least a 2-fold change in expression level relative to the control. In another embodiment, HCP signature genes are defined as genes that show at least a 3-fold change in expression level relative to the control. In a further embodiment, HCP signature genes show at least a 5-fold change in expression level. In a further embodiment, HCP signature genes show at least a 10-fold change in expression level. The change in expression level demonstrated by an HCP signature gene can be an increase in expression or a decrease in expression relative to the control.

The combination of the HCP signature thus identified represents a refined HCP set that can be used to define an expression pattern profile, or “HCP signature,” that is specific to the one or more features of a hematological cancer of interest. This HCP signature can be used as a reference expression pattern considered indicative of one or more feature of a hematological cancer, and thus be compared to the expression pattern obtained from a test sample in order to determine whether the test sample has one or more features of the hematological cancer. The number of genes represented in an HCP signature can vary between about 10 and about 500. In one embodiment, the number of genes represented in an HCP signature is between about 20 and about 300. In another embodiment, the number of genes represented in an HCP signature is between about 30 and about 200. In a further embodiment, the number of genes represented in the HCP signature is between 30 and about 160.

Representative examples of HCP signatures are depicted in Tables 32-41 below.

TABLE 32
DLBCL Signature (≧2-fold relative to control) [51 genes]

| Overexpressed | Underexpressed |
|---|---|
| Symbol | Symbol |
| ABTB2 | ABCB1 |
| BASP1 | BCL2 |
| BIK | BTBD3 |
| CCNE2 | CASP10 |
| CD22 | CDC25B |
| CDCA7 | CLECSF2 |
| CDK4 | CSTA |
| CKS1B | EBI2 |
| CYP27B1 | EPHB6 |
| DNMT3B | GNG11 |
| E2F5 | ICAM2 |
| FAP | IL10RA |
| GRHPR | LYN |
| IL1R1 | MLLT2 |
| LOC90925 | MRPL33 |
| MAD2L1 | PDCD4 |
| MCM4 | PRKCQ |
| MCM7 | RORA |
| MMP12 | SFRS7 |
| MS4A1 | SFRS9 |
| NME1 | TES |
| PARP1 | TGFBR2 |
| PLCG2 | TGFBR3 |
| POLE2 | |
| RGS13 | |
| TERF2 | |
| TOP2A | |
| TRAF1 | |

TABLE 33
FL Signature (≧2-fold relative to control) [70 genes]

| Overexpressed | Underexpressed |
|---|---|
| Symbol | Symbol |
| ABTB2 | ABHD3 |
| ARID5B | ADAM10 |
| BASP1 | ASB7 |
| BIK | BIN2 |
| CCL19 | BZRP |
| CD19 | CASP3 |
| CD22 | CYBB |
| CD24 | DAXX |
| CD81 | EMP3 |
| CDCA7 | HK2 |
| CLU | HSPA6 |
| CR2 | IFI30 |
| CTLA4 | IL15 |
| E2F5 | KLRK1 |
| FCER2 | LGALS1 |
| GCET2 | LYN |
| GPR18 | MRPL33 |
| GYPC | NKG7 |
| IGJ | NPC1 |
| IL1R1 | P4HA1 |
| IRF8 | PRKCD |
| KHDRBS3 | RBBP4 |
| KIAA0746 | SERPINA1 |
| LOC90925 | SFRS7 |
| LPL | SYBL1 |
| MAGED1 | TGFBR3 |
| MCM4 | TIGD7 |
| MS4A1 | TLE1 |
| OSBPL10 | TNFSF10 |
| PARP1 | TYROBP |
| PIK3C2B | WARS |
| POSTN | |
| RGS13 | |
| SPAP1 | |
| TIMD4 | |
| TNFRSF7 | |
| TRAF1 | |
| TRAF5 | |
| UBE2J1 | |

TABLE 34
HL Signature (≧2-fold relative to control) [33 genes]

| Overexpressed | Underexpressed |
|---|---|
| Symbol | Symbol |
| AKR1C1 | 13CDNA73 |
| APOC2 | CDKN2D |
| CCL19 | CSTA |
| CD81 | EBI2 |
| CTLA4 | GNG11 |
| CYP27B1 | HSPA6 |
| DHCR24 | KLRK1 |
| FLJ40504 | LYN |
| GSTT1 | MRPL33 |
| IL1R1 | PISD |
| KIAA0992 | SORL1 |
| LGMN | STS-1 |
| MMP12 | TGFBR3 |
| NFKBIA | |
| PIK3C2B | |
| PRAME | |
| SLAMF1 | |
| STAT1 | |
| TFF3 | |
| TRAF1 | |

TABLE 40
AML Cell Line Signature
(≧5-fold relative to control) [129 genes]

| Overexpressed | | Underexpressed | |
|---|---|---|---|
| | GenBank ™ | | GenBank ™ |
| Symbol | Accession No | Symbol | Accession No |
| BIRC5 | NM_001168.1 | AKAP12 | AI672553.1 |
| BZRP | NM_007311.2 | ANXA1 | NM_000700.1 |
| C13orf18 | NM_025113.1 | APOBEC3G | NM_021822.1 |
| C3orf6 | AK055652.1 | ASPM | NM_018136.2 |
| CDC20 | NM_001255.1 | AUTS2 | NM_015570.1 |
| CDC6 | NM_001254.2 | BASP1 | NM_006317.3 |
| CDCA7 | NM_031942.3 | BCL2A1 | NM_004049.2 |
| CDKN3 | NM_005192.2 | BIRC3 | NM_001165.3 |
| CHEK1 | NM_001274.2 | BTK | NM_000061.1 |
| CLIC5 | AL049313.1 | CASP4 | AL050391.1 |
| CSF1R | NM_005211.2 | CCL5 | NM_002985.2 |
| DMD | NM_000109.2 | CCNA2 | NM_001237.2 |
| FGR | NM_005248.1 | CD2 | NM_001767.2 |
| HCK | NM_002110.2 | CD24 | NM_013230.1 |
| IL16 | NM_004513.3 | CD36 | NM_001001547.1 |
| KNTC2 | NM_006101.1 | CD3D | NM_000732.2 |
| LGALS1 | NM_002305.2 | CD44 | NM_000610.3 |
| LGALS3 | NM_002306.1 | CD79B | NM_000626.1 |
| LILRB1 | AI523104.1 | CD86 | NM_006889.2 |
| NKG7 | NM_005601.3 | CDC2 | NM_001786.2 |
| PCDH9 | BC042366.1 | CDCA1 | NM_031423.2 |
| PLA2G7 | NM_005084.2 | CR2 | NM_001877.2 |
| PLCG2 | NM_002661.1 | CSF3R | NM_156038.2 |
| PON2 | NM_000305.1 | CSTA | NM_005213.2 |
| PPBP | NM_002704.2 | CTGF | NM_001901.1 |
| PRKCD | NM_006254.3 | CX3CR1 | NM_001337.2 |
| PTPN6 | NM_002831.3 | CXCR4 | NM_003467.1 |
| PTPRC | NM_002838.2 | DNTT | NM_004088.2 |
| RAB13 | NM_002870.2 | EBI2 | NM_004951.2 |
| RAG2 | NM_000536.1 | ENPP2 | NM_006209.2 |
| S100A6 | NM_014624.2 | ENTPD1 | NM_001776.3 |
| SERPINA1 | NM_000295.3 | FCER2 | NM_002002.3 |
| SFRS3 | BE927772.1 | FLJ40504 | NM_173624.1 |
| SPAP1 | NM_030764.2 | GUCY1A3 | NM_000856.1 |
| SPIB | NM_003121.1 | HCLS1 | NM_005335.3 |
| TCF4 | AI307786.1 | HIST1H2AC | BC050602.1 |
| TCF7 | NM_201632.1 | HIVEP2 | NM_006734.2 |
| TIMP1 | NM_003254.1 | HLA-DMA | NM_006120.2 |
| TLE1 | NM_005077.3 | HLA-DPA1 | NM_033554.2 |
| | | HLA-DPB1 | NM_002121.4 |
| | | HLA-DRA | NM_019111.2 |
| | | IFI30 | NM_006332.3 |
| | | IFI44L | NM_006820.1 |
| | | IGJ | NM_144646.2 |
| | | IL10RA | NM_001558.2 |
| | | KIF2C | NM_006845.2 |
| | | KLRK1 | NM_007360.1 |
| | | KRT19 | NM_002276.3 |
| | | LCK | NM_005356.2 |
| | | LTB | NM_002341.1 |
| | | LY86 | NM_004271.1 |
| | | MAD2L1 | NM_002358.2 |
| | | MAL | NM_022438.1 |
| | | MBNL1 | NM_021038.3 |
| | | MGMT | NM_002412.2 |
| | | MS4A1 | NM_021950.2 |
| | | MT1G | NM_005950.1 |
| | | MYL4 | W04885.1 |
| | | NAPSA | NM_004851.1 |
| | | NCF4 | NM_013416.2 |
| | | P2RY5 | NM_005767.3 |
| | | PF4 | NM_002619.1 |
| | | PLAU | NM_002658.1 |
| | | POLE2 | NM_002692.2 |
| | | PRAME | NM_006115.3 |
| | | PSPH | NM_004577.3 |
| | | PTPRCAP | NM_005608.1 |
| | | RGL1 | NM_015149.2 |
| | | RIPK2 | NM_003821.4 |
| | | RNASE1 | NM_198232.1 |
| | | S100A12 | NM_005621.1 |
| | | S100A4 | NM_002961.2 |
| | | SAMHD1 | NM_015474.2 |
| | | SERPINB1 | NM_030666.2 |
| | | SERPING1 | NM_000062.1 |
| | | SESN3 | BU929213.1 |
| | | SLA | NM_006748.1 |
| | | SLAMF1 | NM_003037.1 |
| | | SLC1A4 | NM_003038.2 |
| | | SORL1 | U90916.1 |
| | | STMN1 | NM_005563.3 |
| | | SWAP70 | AB014540.1 |
| | | TCL1A | NM_021966.1 |
| | | TFRC | NM_003234.1 |
| | | TOP2A | NM_001067.2 |
| | | TRIP6 | NM_003302.1 |
| | | TXNIP | NM_006472.1 |
| | | TYROBP | NM_003332.2 |
| | | VEGF | BC058855.1 |
| | | ZAP70 | NM_001079.3 |

TABLE 41
T-ALL Cell Line Signature
(≧5-fold relative to control) [111 genes]

| Overexpressed | | Underexpressed | |
|---|---|---|---|
| | GenBank ™ | | GenBank ™ |
| Symbol | Accession No | Symbol | Accession No |
| BIRC5 | NM_002102.2 | AKAP12 | W04885.1 |
| C13orf18 | NM_001168.1 | ANXA1 | NM_013230.1 |
| C3orf6 | NM_001255.1 | APP | NM_173624.1 |
| CD74 | NM_006101.1 | ASPM | BC058855.1 |
| CDC20 | NM_001254.2 | AUTS2 | NM_002358.2 |
| CDC6 | NM_001274.2 | BASP1 | NM_018136.2 |
| CHEK1 | NM_000536.1 | BCL2A1 | NM_006845.2 |
| CLIC5 | NM_004088.2 | BIRC3 | NM_031966.2 |
| CSF1R | NM_201632.1 | BTK | NM_001237.2 |
| DNTT | NM_005601.3 | CASP4 | NM_001067.2 |
| FGR | NM_002838.2 | CCL5 | NM_003234.1 |
| GYPE | AL049313.1 | CCNA2 | NM_001786.2 |
| IL16 | AI307786.1 | CCNB1 | NM_002692.2 |
| KNTC2 | NM_000305.1 | CD2 | NM_004577.3 |
| LILRB1 | AK055652.1 | CD24 | NM_022438.1 |
| NKG7 | NM_002870.2 | CD36 | NM_006748.1 |
| PCDH9 | NM_025113.1 | CD37 | NM_004615.2 |
| PLCG2 | NM_002661.1 | CD3D | NM_000700.1 |
| PON2 | NM_003121.1 | CD44 | NM_000732.2 |
| PTPRC | NM_004513.3 | CD86 | NM_003467.1 |
| RAB13 | AI523104.1 | CDC2 | NM_001767.2 |
| RAG2 | NM_004355.1 | CNN3 | NM_005356.2 |
| SERPINA1 | BC042366.1 | CSF3R | NM_006734.2 |
| SPIB | NM_005248.1 | CSTA | NM_001079.3 |
| TCF4 | NM_001242.3 | CTGF | NM_005608.1 |
| TCF7 | NM_005211.2 | CXCR4 | NM_005531.1 |
| TNFRSF7 | NM_000295.3 | EBI2 | NM_012151.2 |
| | | ENG | AI672553.1 |
| | | ENPP2 | BU929213.1 |
| | | ENTPD1 | NM_005965.3 |
| | | F8A1 | NM_000237.1 |
| | | FCER2 | NM_001839.2 |
| | | FLJ40504 | NM_006209.2 |
| | | FN1 | NM_000062.1 |
| | | HCK | NM_212474.1 |
| | | HCLS1 | NM_001901.1 |
| | | HIST1H2AC | NM_003302.1 |
| | | HIVEP2 | NM_003821.4 |
| | | HLA-DMA | BC050602.1 |
| | | HLA-DPA1 | NM_015149.2 |
| | | HLA-DPB1 | NM_021966.1 |
| | | HLA-DRA | NM_002002.3 |
| | | IFI16 | NM_006317.3 |
| | | IFI30 | NM_006120.2 |
| | | IFI44L | NM_001774.1 |
| | | IGJ | NM_144646.2 |
| | | IL10RA | NM_021950.2 |
| | | KIF2C | NM_002341.1 |
| | | KLRK1 | NM_013416.2 |
| | | LCK | NM_005213.2 |
| | | LPL | NM_015570.1 |
| | | LTB | U90916.1 |
| | | LY86 | NM_004049.2 |
| | | MAD2L1 | NM_015474.2 |
| | | MAL | NM_004951.2 |
| | | MBNL1 | NM_003037.1 |
| | | MS4A1 | NM_156038.2 |
| | | MYL4 | NM_001776.3 |
| | | MYLK | NM_004851.1 |
| | | NAPSA | NM_000484.2 |
| | | NCF4 | NM_006820.1 |
| | | POLE2 | NM_000061.1 |
| | | PSPH | NM_002985.2 |
| | | PTPRCAP | NM_001558.2 |
| | | RGL1 | NM_000610.3 |
| | | RIPK2 | NM_004271.1 |
| | | S100A12 | AB014540.1 |
| | | S100A4 | NM_001165.3 |
| | | SAMHD1 | NM_006889.2 |
| | | SERPING1 | NM_002110.2 |
| | | SESN3 | NM_005335.3 |
| | | SLA | NM_007360.1 |
| | | SLAMF1 | NM_000118.1 |
| | | SORL1 | AL050391.1 |
| | | SWAP70 | NM_001001547.1 |
| | | TCL1A | NM_005621.1 |
| | | TFRC | NM_021038.3 |
| | | TM4SF2 | NM_006332.3 |
| | | TOP2A | NM_002961.2 |
| | | TRIP6 | NM_006472.1 |
| | | TXNIP | NM_003332.2 |
| | | TYROBP | NM_033554.2 |
| | | VEGF | NM_002121.4 |
| | | ZAP70 | NM_019111.2 |

TABLE 35
MCL Signature (≧2-fold relative to control) [80 genes]

| Overexpressed | Underexpressed |
|---|---|
| Symbol | Symbol |
| 3.8-1 | 13CDNA73 |
| ADAM19 | ABHD3 |
| ARHGAP24 | AHR |
| BANK1 | BIN2 |
| BASP1 | CAMK1 |
| C13orf18 | CCND3 |
| C3orf6 | CD58 |
| CACNB2 | CD86 |
| CD19 | CSTA |
| CD22 | FAS |
| CD24 | GATA3 |
| CD79B | HBS1L |
| CELSR1 | IFI30 |
| CR2 | IL1B |
| DNMT3A | LGALS2 |
| E2F5 | MAPKAPK3 |
| GM2A | MLLT3 |
| GTF2E2 | MRPL33 |
| HLA-DOB | NKG7 |
| ITGAD | NUCB2 |
| KCNN4 | PISD |
| KIAA0355 | PRKCQ |
| KIAA0746 | S100A10 |
| LOC55565 | SERPINA1 |
| LOC90925 | SERPINB1 |
| LPL | SERPINB8 |
| MCM7 | SLAMF1 |
| MGC39372 | SORL1 |
| MME | STAT5B |
| KIAA0504 | TNFSF10 |
| MS4A1 | TYROBP |
| MYH11 | WARS |
| NAPSA | |
| NFATC1 | |
| NFKBIE | |
| OSBPL10 | |
| PARP1 | |
| PDLIM1 | |
| PIK3C2B | |
| PLCG2 | |
| RASA1 | |
| SPAP1 | |
| SYK | |
| TNFRSF13B | |
| TNFRSF7 | |
| TRAF5 | |
| UNG | |
| ZNF177 | |

TABLE 36
MZL Signature (≧2-fold relative to control) [159 genes]

| | |
|---|---|
| Overexpressed | Underexpressed |
| SYMBOLol | Symbol |

| | |
|---|---|
| ABTB2 | 13CDNA73 |
| AKR1C1 | ABCB1 |
| APOC2 | ABHD3 |
| APOD | ANXA5 |
| ARID5B | APC |
| BAIAP2L1 | APOBEC3G |
| BASP1 | ARF6 |
| BIK | ARID4A |
| BTG2 | ASB7 |
| C13orf18 | BCL2A1 |
| C3orf6 | BLVRB |
| CCL19 | C5orf15 |
| CD19 | CAMK1 |
| CD209L | CASP3 |
| CD22 | CCND3 |
| CD24 | CCNG2 |
| CD72 | CCR5 |
| CD79B | CCT6A |
| CDCA7 | CD86 |
| CDK4 | CLECSF2 |
| COL9A2 | CYBB |
| COL9A3 | DCK |
| CR2 | EIF4A2 |
| CRYM | ELF1 |
| CSNK1E | FKBP5 |
| CTSD | GLUD1 |
| DBN1 | GMPS |
| DHCR24 | GNG10 |
| DMD | GNG11 |
| DNMT3B | HIST1H2BK |
| DVL2 | HSPCA |
| E2F5 | HSPH1 |
| EPHB6 | IL15 |
| FCER2 | IL8 |
| FLJ40504 | IQWD1 |
| FSCN1 | ITGB1 |
| GM2A | KLRK1 |
| GSTT1 | LGALS3 |
| HIP1R | LOC54103 |
| HLA-DOB | LTBP1 |
| HLA-DQA1 | LYN |
| IGHM | MAFB |
| IGJ | MAPK9 |
| ITGAD | MGC5395 |
| ITIH3 | MLLT2 |
| KCNN4 | MYC |
| KHDRBS3 | NFE2L2 |
| KIAA0746 | NFE2L3 |
| KRT5 | NKG7 |
| LOC55565 | NPC1 |
| LOC90925 | NT5E |
| LPL | NUCB2 |
| MAGED1 | P4HA1 |
| MCM7 | PBX3 |
| MME | PIK3CA |
| MS4A1 | PPM1B |
| NFATC1 | PRDM2 |
| NME1 | PROS1 |
| OSBPL10 | PTPRC |
| PIK3C2B | RBBP4 |
| PLK3 | RBM12 |
| PRKCB1 | RORA |
| PTPN7 | S100A10 |
| RASA1 | SAFB |
| RASGRF1 | SELL |
| RGS13 | SERPINA1 |
| SH3BP5 | SERPINB1 |
| SLC38A3 | SFRS9 |
| SPAP1 | SORL1 |
| SYK | STAT3 |
| TCL1A | SYBL1 |
| TFF3 | TEGT |
| TNFRSF13B | TES |
| TNFRSF7 | TNFSF10 |
| TRAF5 | TOP2B |
| TYMS | TYROBP |
| WNT10A | UBE2D2 |
| WNT5B | WAC |
| YWHAE | WARS |
| | ZBTB16 |

TABLE 37
SLL Signature (≧2-fold relative to control) [128 genes]

| Overexpressed | Underexpressed |
|---|---|
| Symbol | Symbol |
| 3.8-1 | ABCB1 |
| AKAP12 | ABHD3 |
| AKR1C1 | ADAM10 |
| ARID5B | ANXA5 |
| BIK | BCL2A1 |
| C13orf18 | BID |
| C3orf6 | BIRC2 |
| CACNB2 | BTBD3 |
| CCL19 | CAMK1 |
| CCNE1 | CASP3 |
| CD19 | CCNG2 |
| CD209L | CCR5 |
| CD22 | CD86 |
| CD24 | CLU |
| CD72 | CSF1R |
| CD79B | CSTA |
| CDK4 | CYBB |
| COL9A2 | EBI2 |
| COL9A3 | EIF4A2 |
| CR2 | FKBP5 |
| CSNK1E | GATA3 |
| CTSD | HK2 |
| DMD | HLA-DRB1 |
| DNMT3B | HSPA5 |
| E2F5 | HSPH1 |
| FCER2 | IFI30 |
| FLJ40504 | IL15 |
| CS0DC015YK09 | IL1B |
| HIVEP2 | IL8 |
| HLA-DOB | IQWD1 |
| HLA-DQA1 | ITGB1 |
| IGFBP4 | LGALS1 |
| IRF8 | LGALS2 |
| KCNN4 | LGALS3 |
| KIAA0746 | LTBP1 |
| LOC55565 | MLLT2 |
| LPL | MLLT3 |
| MS4A1 | MYC |
| MYH11 | NFE2L2 |
| NFATC1 | NKG7 |
| NFKBIE | NPC1 |
| NME1 | NUCB2 |
| OSBPL10 | P4HA1 |
| PBP | PRKCD |
| PLK3 | PTPRC |
| POLE2 | RBM12 |
| PRKCB1 | RORA |
| PTPN7 | S100A10 |
| RASA1 | SAFB |
| RASGRF1 | SERPINA1 |
| SH3BP5 | SERPINB1 |
| SLC38A3 | SFRS9 |
| SPAP1 | SLC20A1 |
| SYK | SORL1 |
| TCL1A | STAT5B |
| TFF3 | SYBL1 |
| TNFRSF7 | TEGT |
| TOSO | TES |
| TRAF5 | TGFBR2 |
| UNG | TIMP1 |
| WNT10A | TNFSF10 |
| ZNF177 | TOP2B |
| | TRA1 |
| | TYROBP |
| | WARS |
| | ZBTB16 |

TABLE 38
TCL Signature (≧2-fold relative to control) [92 genes]

| Overexpressed | Underexpressed |
|---|---|
| Symbol | Symbol |
| AKR1C1 | 13CDNA73 |
| BAD | BACH2 |
| CCL19 | BANK1 |
| CCL8 | BCL6 |
| CDC6 | BTK |
| CDCA4 | CCNG2 |
| CDK4 | CD69 |
| CDKN1A | CD79B |
| CKS1B | CLECSF2 |
| CKS2 | CYBB |
| DBN1 | DGKD |
| DHCR24 | EBI2 |
| DNMT3B | EIF4A2 |
| DXS9879E | FCER2 |
| E2F2 | IFI16 |
| FAP | ITGB1 |
| GSTT1 | KLRK1 |
| H2AFX | LOC54103 |
| HLA-DQA1 | LOC90925 |
| IL15RA | LTBP1 |
| IL6 | LY64 |
| KIAA0992 | LY86 |
| LARGE | LYN |
| MAD2L1 | MIR |
| MCM4 | MLLT2 |
| MT1G | MS4A1 |
| MT1H | NAP1L1 |
| NID2 | NAPSA |
| NME1 | NFATC1 |
| PARD3 | NFE2L2 |
| PNAS-4 | NUCB2 |
| POLE2 | PDCD4 |
| POSTN | PRDM2 |
| PSPH | PTPRC |
| RAMP3 | SAFB |
| SLC38A3 | SELL |
| TIMP1 | SERPINB1 |
| TPM4 | SFRS9 |
| UBE2J1 | SORL1 |
| WNT5B | SP110 |
| | SPAP1 |
| | STARD7 |
| | SWAP70 |
| | SYK |
| | TCL1A |
| | TEGT |
| | TES |
| | TGFBR2 |
| | TGFBR3 |
| | TLE1 |
| | TNFSF10 |
| | ZNF91 |

TABLE 39
CLL Signature (≧5-fold relative to control) [66 genes]

| | |
|---|---|
| Overexpressed | Underexpressed |

| | GenBank ™ | | GenBank ™ |
|---|---|---|---|
| Symbol | Accession No. | Symbol | Accession No |
| BAIAP2L1 | NM_018842.3 | ANXA1 | NM_000700.1 |
| C3orf6 | AK055652.1 | BCL2A1 | NM_000484.2 |
| CD74 | NM_004355.1 | CCNA2 | NM_004049.2 |
| CDCA7 | NM_031942.3 | CD24 | NM_001237.2 |
| CDKN3 | NM_005192.2 | CDC2 | NM_013230.1 |
| CLIC5 | NM_016184.2 | CSF3R | NM_001786.2 |
| CNN3 | AL049313.1 | ENTPD1 | NM_156038.2 |
| COL1A2 | NM_001839.2 | EN1 | NM_001776.3 |
| CX3CR1 | NM_000089.3 | APP | NM_212474.1 |
| ENPP2 | NM_001337.2 | STMN1 | NM_005531.1 |
| GATA3 | NM_006209.2 | IFI16 | NM_001558.2 |
| HCK | NM_002051.2 | IL10RA | NM_007360.1 |
| IGFBP7 | NM_002110.2 | KLRK1 | NM_000237.1 |
| ITGAL | NM_001553.1 | LPL | NM_002341.1 |
| LGALS2 | T28925.1 | LTB | NM_002358.2 |
| LGALS3 | NM_006498.1 | MAD2L1 | NM_021038.3 |
| LOC54103 | NM_002306.1 | MBNL1 | NM_002619.1 |
| MT1H | AI523104.1 | PF4 | NM_002692.2 |
| NK4 | T66903.1 | POLE2 | NM_006115.3 |
| NKG7 | NM_005951.1 | PRAME | NM_004577.3 |
| PLA2G7 | NM_004221.3 | PSPH | NM_005608.1 |
| PLAU | NM_005601.3 | PTPRCAP | NM_015149.2 |
| PON2 | NM_005084.2 | RGL1 | NM_003821.4 |
| PPBP | NM_002658.1 | RIPK2 | NM_005621.1 |
| PRKCD | NM_000305.1 | S100A12 | NM_006748.1 |
| SPAP1 | NM_002704.2 | SLA | U90916.1 |
| SPIB | NM_006254.3 | SORL1 | NM_005563.3 |
| STAT1 | NM_030764.2 | SWAP70 | AB014540.1 |
| TCF4 | NM_003121.1 | TFRC | NM_003234.1 |
| TLE1 | AK022231.1 | TRAF1 | NM_005658.2 |
| TNFSF10 | AI307786.1 | ZAP70 | NM_001079.3 |
| TRIP6 | NM_005077.3 | | |
| TYROBP | NM_003810.2 | | |
| CLECSF6 | NM_003302.1 | | |
| LILRB1 | NM_003332.2 | | |

### 4. Profiling of Hematological Cancers

The HCP system of the present invention provides for the profiling of a hematological cancer in a subject with respect to one or more features of the hematological cancer. Hematological cancers that can be profiled using the system of the present invention are selected from the group of lymphoma or leukemia. The information provided by the hematological cancer profiling methods has a number of applications, for example, in disease prognosis, diagnosis, staging or grading, treatment management, monitoring of disease progression, predicting disease outcome or complications, pharmacogenomics, and the like. Treatment management, for example, includes selecting the most appropriate drug(s) or other therapy for effective treatment of the hematological cancers. Predicting disease outcome can involve, for example, predicting a patient's chance of survival, relapse, or the likelihood of disease progression.

For example, the expression pattern of genes in one or more HCP sets in a test sample obtained from a patient suspected of having a hematological cancer can be compared to the expression pattern of genes in the same HCP set(s) in samples from control subjects. Examples of control subjects include, but are not limited to, subjects known to have a specific hematological cancer, healthy subjects, subjects having a hematological cancer at a defined stage or grade, subjects having drug-resistant, multi-drug resistant or drug-sensitive hematological cancer, subjects undergoing a defined chemotherapy regimen, untreated subjects having a hematological cancer, subjects having a specific subtype of a hematological cancer, and the like. Similarly, for some treatments with known side effects, the HCP combination can be employed to “fine tune” the treatment regimen. A dosage is established that causes a change in gene expression patterns indicative of successful treatment. Expression patterns associated with undesirable side effects are avoided. This approach may be more sensitive and rapid than waiting for the patient to show inadequate improvement, or manifest symptoms, before altering the course of treatment.

As the HCP combinations of the present invention allow for the simultaneous investigation of several features of a hematological cancer in a single assay, the information obtained from a single assay may be applicable in several areas. Thus, the HCP system of the present invention provides for methods of diagnosing a hematological cancer, typing a hematological cancer, monitoring progression of a hematological cancer, monitoring the rate of progression of a hematological cancer, predicting therapeutic outcome, determining prognosis, monitoring response to treatment, predicting survival, selecting appropriate drug(s) and/or therapies for treatment, and combinations thereof.

In addition, the present invention also contemplates that the methods can be used in drug discovery and development, toxicological and carcinogenicity studies, forensics, and the like, wherein the test sample may be taken from either an in vitro cell culture, or from a human or other animal subject. For example, expression patterns relating to the effects of currently available therapeutic drugs can be investigated. Test samples obtained from individuals treated with these drugs can be analyzed and compared to samples from untreated patients. In this way, an expression pattern of known therapeutic agents will be developed. Knowing the identity of sequences that are differentially regulated in the presence and absence of a drug will allow researchers to elucidate the molecular mechanisms of action of that drug. Similarly, large numbers of candidate drugs can be screened based on the expression pattern similar to those of known therapeutic drugs, with the expectation that molecules with the same expression profile will likely have similar therapeutic effects. Thus, the invention provides the means to determine the molecular mode of action of a drug.

### 5. Other Uses of the HCP Combinations

The HCP system of the present invention further provides for the nucleotide sequences of the HCP combination in computer-readable media for in silico applications and as a basis for the design of gene-specific primers for amplification of one or more genes of an HCP set.

Gene-specific primers based on the nucleotide sequences of genes of the HCP sets of the system can be designed for use in amplification of the genes of the HCP set. For use in amplification reactions such as PCR, a pair of primers will be used. The exact composition of the primer sequences is not critical to the invention, but for most applications the primers will hybridize to specific genes of the HCP set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess gene expression levels of genes of the HCP set. Alternatively, these primers may be used in combination with probes, such as molecular beacons, as described supra, in amplifications using real-time PCR.

### 6. HCP Profiling Kits and Packages and Computer-Readable Media

The present invention further provides for kits and packages for the profiling of hematological cancers in a subject comprising the probes of an HCP combination. The probes can be provided in the kit immobilized to a solid substrate (i.e. as an HCP array) or in the form of isolated nucleic acids suitable for use in solution hybridization assays, or in the preparation of an HCP array. Where appropriate, the HCP probes provided in the kit may incorporate a detectable label, such as a fluorophore, radioactive moiety, enzyme, biotin/avidin label, chromophore, chemiluminescent label, or the like, or the kit may include reagents for labeling the probes. The probes can be provided in separate containers or pre-dispensed into a convenient format for subsequent use, for example, into microtitre plates.

The kits can optionally include additional reagents, such as buffers, salts, enzymes, enzyme co-factors, substrates, labels, detection reagents, and the like. Other components, such as buffers and solutions for the isolation, treatment or amplification of a test sample, may also be included in the kit, for example, the kit may contain reagents for reverse transcription, transcription and/or PCR, including enzymes, primers and nucleotides. The kit may additionally include one or more controls. One or more of the components of the kit may be lyophilised and the kit may further comprise reagents suitable for the reconstitution of the lyophilised components.

The various components of the kit are provided in suitable containers. Where appropriate, the kit may also optionally contain reaction vessels, mixing vessels and other components that facilitate the preparation of reagents or the test sample.

The kit can optionally include instructions for use, which may be provided in paper form or in computer-readable form, such as a disc, CD, DVD or the like.

The present invention further contemplates that the kits described above may be provided as part of a package that includes computer software to analyze the gene expression patterns generated from the use of the kit.

The present invention further provides a computer-readable medium comprising one or more digitally-encoded HCP signatures, each signature being associated with one or more values and each value representing the expression of a gene represented by the HCP signature which is correlated with a feature of the hematological cancer represented by the HCP signature. Such a computer-readable medium can be used as a reference to compare the expression profile for the genes of a HCP signature generated from profiling a test sample. The digitally-encoded HCP signatures can be comprised in a database (such as the database described in U.S. Pat. No. 6,308,170).

The invention will now be described with reference to specific examples. It will be understood that the following examples are intended to describe embodiments of the invention and are not intended to limit the invention in any way.

### EXAMPLES

### Example 1

### Selection of Genes for an HCP Set Specific for DLBCL

Genetic profiling studies of hundreds of DLBCL patient samples has distinguished several subtypes of DLBCL: a type derived from differentiated activated peripheral blood b-cells (ABC), a second type derived from the undifferentiated germinal centers of lymph nodes (GC), a third type called Mediastinal Large B-Cell Lymphoma (MLBCL), and a fourth category that remains largely heterogeneous (Rosenwald A, Wright G, Chan W C et al. N Engl J Med. 2002; 346(25):1937-47; Rosenwald A, Wright G, Leroy K. et al. J Exp Med. 2003; 198(6):851-62; Wright G, Tan B, Rosenwald A, et al. Proc Natl Acad Sci USA. 2003; 100(17):9991-6; Savage K J, Monti S, Kutok J L, et al. Blood. 2003; 102(12):3871-9). These studies show that patients with GC-DLBCL have a significantly better survivability than patients with ABC-DLBCL. Savage et al. also presented a genetic signature for MLBCL that allows it to be distinguished from DLBCL.

Genes specific for DLBCL were selected by mining public databases. The genes are listed in Table 6 and include genes in the following categories.

ABC vs. GC DLBCL: These genes distinguish between ABC and GC subtypes of DLBCL. Each one of these two subtypes of DLBCL express genes with a specific expression pattern. The genes chosen for this section are unique to either ABC or GC DLBCL and can therefore be used to distinguish the two diseases, as described in Alizadeh A A, Eisen M B, Davis R E et al. Nature. 2000; 403(6769):503-11; Rosenwald A, Wright G, Chan W C et al. N Engl J Med. 2002; 346(25):1937-47; Wright G, Tan B, Rosenwald A, et al. Proc Natl Acad Sci USA. 2003; 100(17):9991-6; Shipp M A, Ross K N, Tamayo P et al. Nat Med. 2002 January; 8(1):68-74; and Pan Z, Shen Y, Du C, et al. Am J Pathol. 2003; 163(1):135-44.

DLBCL vs. FL: FL is closely related to DLBCL. The genes in the DLBCL vs. FL section allow identification of the two major types of disease, as well as give us the ability to predict whether or not a particular FL will progress into the more aggressive DLBCL (Shipp M A, Ross K N, Tamayo P et al. Nat Med. 2002 January; 8(1):68-74; Lossos I S, Alizadeh A A, Diehn M, et al. Proc Natl Acad Sci USA. 2002; 99(13):8886-91; de Vos S, Hofmann W K, Grogan T M, et al. Lab Invest. 2003; 83(2):271-85).

MLBCL vs. DLBCL: Similar to the last category, these genes separate DLBCL from Mediastinal Large B-cell Lymphoma (Savage K J, Monti S, Kutok J L, et al. Blood 2003; 102(12):3871-9; Kossakowska A E, Urbanski S J, Watson A, et al. Oncol Res. 1993; 5(1):19-28).

SURVIVAL: Shipp et al. correlated patient survival with a group of 96 marker genes; these genetic markers have the ability to predict a patient's life expectancy with significance and have therefore been included in our chip (Shipp M A, Ross K N, Tamayo P et al. Nat Med. 2002 January; 8(1):68-74).

Other genes, such as those described in Nishiu M, Yanagawa R, Nakatsuka S, et al. Jpn J Cancer Res. 2002; 93(8):894-901, can also be selected indicate the advancement of DLBCL within the sample.

### Example 2

### Selection of Genes for an HCP Set Specific for FL

Follicular lymphoma is an indolent or slow growing cancer that is the result of a t(14;18) translocation mutation in genomic DNA. FL accounts for 25-40% of all non-Hodgkin's lymphomas. Though FL is slow growing and 60-70% of patients live longer than five years after diagnosis, it is nonetheless incurable; patients endure continued relapse and decreased sensitivity to therapy until succumbing to disease. FL transforms into a more aggressive form of large b-cell lymphoma in 25-60% of patients (Lossos I S, Alizadeh A A, Diehn M, et al. Proc Natl Acad Sci USA. 2002; 99(13):8886-91).

Lossos et al. performed genetic profiling experiments on FL patients and tracked their progress to observe transformation into DLBCL. They found that follicular lymphomas that transformed into DLBCL de-regulated a particular gene signature that could be identified by microarray analysis. The genes chosen for this section have been shown to predict which cases will transform from FL into DLBCL. Several other teams have investigated the gene expression profile of FL in comparison with DLBCL, Burkitt's lymphoma and normal germinal center B-cells. Some of the genes in this set form a signature pattern enabling diagnostic separation of FL tissue from healthy samples and several other types of lymphoma (Shipp M A, Ross K N, Tamayo P et al. Diffuse large B-cell lymphoma outcome prediction by gene-expression profiling and supervised machine learning. Nat Med. 2002 January; 8(1):68-74; Lossos I S, Alizadeh A A, Diehn M, et al. Proc Natl Acad Sci USA. 2002; 99(13):8886-91; Maesako Y, Uchiyama T, Ohno H. Cancer Sci. 2003: 94(9):774-81; Ghia P, Boussiotis V A, Schultze J L, et al. Blood. 1998; 91(1):244-51; Akasaka T, Lossos I S, Levy R. Blood. 2003; 102(4):1443-8; Elenitoba-Johnson K S, Gascoyne R D, Lim M S, et al. Blood 1998; 91(12):4677-85).

Genes specific for FL were selected by mining public databases. The genes are listed in Table 7 and include genes in the following categories.

DLBCL vs. FL: The genes in the DLBCL vs. FL section allow identification of the two major types of disease, and the prediction of whether or not a particular FL will progress into the more aggressive DLBCL (Shipp M A, Ross K N, Tamayo P et al. Nat Med. 2002 January; 8(1):68-74; Lossos I S, Alizadeh A A, Diehn M, et al. Proc Natl Acad Sci USA. 2002; 99(13):8886-91; Husson H, Carideo E G, Neuberg D, et al. Blood. 2002; 99(1):282-9; Lestou V S, Gascoyne R D, Sehn L, et al. Br J Haematol. 2003; 122(5):745-59; Ghia P, Boussiotis V A, Schultze J L, et al. Blood. 1998; 91(1):244-51).

BCL6 TRANSLOCATION PARTNERS The t(14;18) translocation mutation characteristic of FL deregulates the BCL6 gene. The genes contained in this set are linked closely with BCL6 and thus may be deregulated following the initial mutation. Expression data from these genes may show that they are important in the FL signature (Akasaka T, Lossos I S, Levy R. Blood. 2003; 102(4):1443-8).

FL vs. BURKITT'S Maesako et al., discovered a subset of genes that were deregulated when expression data of either FL or Burkitt's lymphomas were compared. Genes in this set will help separate FL samples from Burkitt's (Maesako Y, Uchiyama T, Ohno H. Cancer Sci. 2003; 94(9):774-81).

### Example 3

### Selection of Genes for an HCP Set Specific for CLL

Chronic Lymphocytic Leukemia is the most common type of human leukemia representing 30% of adult leukemia (Hamblin T J, Davis Z, Gardiner A, et al. Blood. 1999; 94(6):1848-54). In similar fashion to DLBCL and FL, the clinical outcome of CLL patients can vary greatly. Hamblin and his colleagues (1999) discovered that the overall survival of patients afflicted with this disease is correlated with the mutational status of the immunoglobulin V(H) genes; CLL patients whose cells had unmutated Ig V(H) cells had poorer clinical outcomes than patients whose B-cells had already undergone somatic mutation (Hamblin T J, Davis Z, Gardiner A, et al. Blood. 1999; 94(6): 1848-54).

Thus, CLL is a heterogeneous disease with at least two subtypes that are molecularly distinguishable. The genes chosen for this section reflect this hypothesis and have been selected because they were found to be related to both survival and Ig mutational status. Analyzing the expression pattern of genes selected for this set will make it possible to distinguish indolent and aggressive forms, thus assisting clinicians in forming an accurate and suitable treatment regimen (Rosenwald A, Alizadeh A A, Widhopf G, et al. J Exp Med. 2001; 194(11):1639-47; Hamblin T J, Davis Z, Gardiner A, et al. Blood. 1999; 94(6):1848-54; Damle R N, Wasil T, Fais F, et al. Blood. 1999; 94(6):1840-7; Wiestner A, Rosenwald A, Barry T S, et al. Blood. 2003; 101(12):4944-51; Nagy B, Ferrer A, Larramendy M L, et al. Haematologica. 2003; 88(6):654-8; Cro L, Guffanti A, Colombi M, et al. Leukemia. 2003; 17(1):125-32; Thieblemont C, Chettab K, Felman P, et al. Leukemia. 2002; 16(11):2326-9; Korz C, Pscherer A, Benner A, et al. Blood. 2002; 99(12):4554-61).

Genes specific for CLL were selected by mining public databases. The genes are listed in Table 5 and include genes in the following categories.

MUTATIONAL STATUS These genes are differentially regulated between IgV mutated and unmutated CLLs and allow us to differentiate between the two. Since IgV mutation has also been correlated with survival, these genes may also serve as prognostic markers (Rosenwald A, Alizadeh A A, Widhopf G, et al. J Exp Med. 2001; 194(11):1639-47).

CLL vs. DLBCL A number of genes were discovered to distinguish between CLL and DLBCL (Rosenwald A, Alizadeh A A, Widhopf G, et al. J Exp Med. 2001; 194(11):1639-47; Wiestner A, Rosenwald A, Barry T S, et al. Blood. 2003; 101(12):4944-51; Korz C, Pscherer A, Benner A, et al. Blood. 2002; 99(12):4554-61).

CLL vs. MCL Korz et al., performed real time reverse transcription PCR analysis on a number of genes and discovered those that were significantly deregulated between MCL and CLL (Thieblemont C, Chettab K, Felman P, et al. Leukemia. 2002; 16(11):2326-9; Korz C, Pscherer A, Benner A, et al. Blood. 2002; 99(12):4554-61).

### Example 4

### Selection of Genes for an HCP Set Specific for MCL

Mantle Cell Lymphoma affects 6% of non-Hodgkin's patients but accounts for a disproportionately larger number of deaths because it is an incurable malignancy. Within the population affected by the disease, some patients succumb in under a year while others survive for much longer. The median survival for patients with MCL is approximately 3 years (Rosenwald A, Wright G, Leroy K. et al. J Exp Med. 2003; 198(6):851-62). Rosenwald et al. and Martinez et al (Martinez N, Camacho F I, Algara P, et al. Cancer Res. 2003; 63(23):8226-32) have performed gene expression analysis on a number of samples and have correlated survivability with the up or down regulation of certain genes. Rosenwald and his team discovered that by observing the expression of 20 genes that function to govern the proliferation, or rate of growth and multiplication of cells, one is able to predict a patient's survival on a significantly accurate level. Martinez and her team performed a similar study and discovered 25 different genes that were able to perform a similar function.

MCL also has a subtype known as MCL-BV for Mantle Cell Lymphoma Blast Variant. MCL-BV has been shown to be more aggressive and result in poorer clinical outcome than non BV MCL. A paper by de Vos lays out the genes deregulated in MCL-BV that are correlated with more aggressive progression of disease. Other genes in this set have been discovered to be differentially regulated between MCL and other lymphomas such as DLBCL, MZL, and CLL. The expression pattern of genes selected for MCL may allow diagnosis of MCL from a variety of other lymphomas and result in an accurate prognosis based on the expression of certain molecular markers (Rosenwald A, Wright G, Leroy K. et al. J Exp Med. 2003; 198(6):851-62; de Vos S, Hofmann W K, Grogan T M, et al. Lab Invest. 2003; 83(2):271-85; Thieblemont C, Chettab K, Felman P, et al. Leukemia. 2002; 16(11):2326-9; Korz C, Pscherer A, Benner A, et al. Blood. 2002; 99(12):4554-61; Robetorye R S, Bohling S D, Morgan J W, et al. J Mol Diagn. 2002; 4(3):123-36; Martinez N, Camacho F I, Algara P, et al. Cancer Res. 2003; 63(23):8226-32; Kobayashi T, Yamaguchi M, Kim S, et al. Cancer Res. 2003; 63(1):60-6).

Genes specific for MCL were selected by mining public databases. The genes are listed in Table 9 and include genes in the following categories.

SIGNATURE The genes as described in Thieblemont C, Chettab K, Felman P, et al. Leukemia. 2002; 16(11):2326-9; Korz C, Pscherer A, Benner A, et al. Blood. 2002; 99(12):4554-61; Robetorye R S, Bohling S D, Morgan J W, et al. J Mol Diagn. 2002; 4(3):123-36; and Kobayashi T, Yamaguchi M, Kim S, et al. Cancer Res. 2003; 63(1):60-6, have been shown to be deregulated between a MCL and a variety of other lymphomas (MZL, CLL, DLBCL) and thus should be able to distinguish an MCL positive sample.

MCL vs. MCL-BV Genes as described in de Vos S, Hofmann W K, Grogan T M, et al. Lab Invest. 2003; 83(2):271-85, distinguishes between the aggressive MCL-BV or the more indolent form, MCL.

SURVIVAL Certain genetic markers have been correlated with a patient's overall survival after diagnosis. The genes incorporated into this section of the chip will be able to give a patient a prognosis based, not only on morphological studies, but on minute molecular changes (Rosenwald A, Wright G, Leroy K. et al. J Exp Med. 2003; 198(6):851-62; Martinez N, Camacho F I, Algara P, et al. Cancer Res. 2003; 63(23):8226-32).

### Example 5

### Selection of Genes for an HCP Set Specific for HL

Hodgkin's lymphoma has a fairly good prognosis with a 20 percent mortality rate (Devilard E, Bertucci F, Trempat P, et al. Oncogene. 2002; 21(19):3095-102). Diagnosis of this disease still relies on the observance of a particular type of cell morphology. The expression pattern of genes selected for inclusion in this set will allow diagnosis without the need for microscopy (Devilard E, Bertucci F, Trempat P, et al. Oncogene. 2002; 21(19):3095-102; Kuppers R, Klein U, Schwering I, et al. J Clin Invest. 2003; 111(4):529-37; Thorns C, Gaiser T, Lange K, et al. Pathol Int. 2002; 52(9):578-85).

Genes specific for HL were selected by mining public databases. The genes are listed in Table 8 and include genes in the following categories.

SIGNATURE Hodgkin's lymphoma is characterized by the presence of Reed-Sternberg cells. The genes selected for this section are genes that have been compared to various other types of lymphoma and have been found to be specific to Reed Sternberg cells and thus to Hodgkin's lymphoma (Kuppers R, Klein U, Schwering I, et al. J Clin Invest. 2003; 111(4):529-37).

SURVIVAL Devilard et al., related patient survival to a number of genes, all of which have been included in this set (Devilard E, Bertucci F, Trempat P, et al. Oncogene. 2002; 21(19):3095-102).

HL vs. ALCL This section distinguishes HL from a similar non-Hodgkin's lymphoma, ALCL (Thorns C, Gaiser T, Lange K, et al. Pathol Int. 2002; 52(9):578-85).

### Example 6

### Selection of Genes for an HCP Set Specific for ALCL

Anaplastic Large Cell Lymphoma is of T-cell origin and is a disease observed primarily amongst children (Villalva et al., Br J Haematol. 2002; 118(3):791-8). The majority of ALCLs bear a translocation at t(2;5)(p23;q35) that involve both the nucleophosmin (NPM) and anaplastic lymphoma kinase (ALK) genes. A number of diagnostic markers have also been discovered (Thorns C, Gaiser T, Lange K, et al. Pathol Int. 2002; 52(9):578-85; Villalva C, Trempat P, Greenland C, et al. Br J Haematol. 2002; 118(3):791-8; Wellmann A, Thieblemont C, Pittaluga S, et al. Blood. 2000 Jul. 15; 96(2):398-404; Nishikori M, Maesako Y, Ueda C, et al. Blood. 2003; 101(7):2789-96).

Genes specific for ALCL were selected by mining public databases. The genes are listed in Table 4 and include genes in the following categories.

ALCL vs. HL These genes distinguish HL from a similar non-Hodgkin's lymphoma, ALCL (Thorns C, Gaiser T, Lange K, et al. Pathol Int. 2002; 52(9):578-85).

ALK+ vs. ALK− These genes make a distinction between ALK bearing ALCLs from ALCL lacking samples (Villalva C, Trempat P, Greenland C, et al. Br J Haematol. 2002; 118(3):791-8).

### Example 7

### Design and Selection of Nucleotide Sequences Suitable for the Preparation of Polynucleotide Probes from an HCP Set

Nucleotide sequences suitable for the preparation of polynucleotide probes from an HCP set were selected based on the following criteria. The sequences selected were based on regions of the target gene in the HCP set that were 30 or 50 nucleotides in length, and had between 25 and 75% G+C content. The sequences were additionally selected such that self-complementary interactions were minimized. Nucleotide sequences thus selected that can be used for preparation of polynucleotide probes are found in Tables 25-28.

### Example 8

### Selection of Polynucleotide Probes for Preparation of a Small 50Mer Nucleic Acid Array

234 50mer polynucleotide probes were selected for the preparation of a small array for profiling lymphoma. Polynucleotide probes to be used in the preparation of this array were selected as described in example 7, and were designed to be 50 nucleotides in length. The nucleotide sequences of the polynucleotide probes selected for the preparation of this array are listed in Table 25.

### Example 9

### Selection of Polynucleotide Probes for Preparation of a Small 30Mer Nucleic Acid Array

248 30mer polynucleotide probes were selected for the preparation of a small array for profiling lymphoma. Polynucleotide probes to be used in the preparation of this array were selected as described in example 7, and were designed to be 30 nucleotides in length. The nucleotide sequences of the polynucleotide probes selected for the preparation of this array are listed in Table 26.

### Example 10

### Selection of Sequences for/Preparation of a Large 50Mer Nucleic Acid Array

971 50mer polynucleotide probes were selected for the preparation of a large array for profiling lymphoma. Polynucleotide probes to be used in the preparation of this array were selected as described in example 7, and were designed to be 50 nucleotides in length. Other polynucleotide probes corresponding to genes diagnostic of lymphoma on a general level were designed and selected according to methods known in the art. All polynucleotide probe sequences chosen for the large 50mer nucleic acid array are listed in Table 27.

### Example 11

### Selection of Sequences for/Preparation of a Large 30Mer Nucleic Acid Array

971 30mer polynucleotide probes were selected for the preparation of a large array for profiling lymphoma. Polynucleotide probes to be used in the preparation of this array were selected as described in example 7, and were designed to be 30 nucleotides in length. Other polynucleotide probes corresponding to genes diagnostic of lymphoma on a general level were designed and selected according to methods known in the art. All of the polynucleotide probe sequences chosen for the large 50mer nucleic acid array are listed in Table 28.

### Example 12

### Preparation of a Nucleic Acid Array

Small or large nucleic acid arrays can be prepared using the similar protocol.

A small 50mer nucleic acid array was prepared using UltraGAPS™ slides (Corning Life Sciences) as follows. Oligonucleotide probes with the sequences as set forth in Table 25 were synthesized and purified according to standard methods and were spotted in duplicate onto the slides at a concentration of 0.50 mg/mL using Corning's Pronto!™ Universal Spotting Solution. The slides were then incubated in a dessicator for 24-48 hours. Probes were immobilized onto the GAPS-coated surface of the slide by UV cross-linking.

### Example 13

### Preparation of Test Samples

A test sample, either a tissue or blood sample, obtained from a patient having, suspected of having, or suspected of being at risk for a hematological cancer can be prepared for use with a hematological cancer profiling array as follows.

RNA Isolation (with Qiagen RNeasy Kit, Boom et al., 1990)

1) Approximately 20-30 mg tissue is placed in liquid nitrogen and ground thoroughly using a mortar and pestle. The ground powder and liquid nitrogen are poured into a cooled 2 ml microfuge tube. The liquid nitrogen is allowed to evaporate off without letting the samples thaw out. Approximately 600 μl of buffer RLT are added in order to disrupt the cells.

2) Following the disruption of the cells by buffer RLT containing β-ME and guanidine thiocyanate, the cells are homogenized by passage through a 20 gauge needle.

3) The tissue lysate is spun for 3 min at maximum speed in a microcentrifuge. The supernatant is carefully transferred to a new microcentrifuge tube by pipetting. Only the supernatant (lysate) is used in subsequent steps.

4) Approximately 600 μl of 70% ethanol to the homogenized lysate is added and, mixed well by pipetting. It is not centrifuged as it would cause precipitation and decrease in RNA yield.

5) Up to approximately 700 μl of the sample, including any precipitate that may have formed, is added to an RNeasy mini column placed in a 2 ml collection tube (supplied in the kit). The column is centrifuged for 15 s at ≧8000×g (≧10,000 rpm) and flow through is discarded. The collection tube is reused in step 6.

6) Approximately 700 μl Buffer RW1 is then added to the RNeasy column and centrifuged for 15 s at ≧8000×g (≧10,000 rpm) to wash the column. Flow through is discarded alongside of the collection tube used for the first wash stage.

7) The RNeasy column is placed into a new 2 ml collection tube (supplied). Approximately 500 μl of Buffer RPE is pipetted onto the column and it is centrifuged for 15 s at ≧8000×g (≧10,000 rpm) to wash. Flow-through is again discarded.

8) A second volume of 500 μl Buffer RPE is added to the RNeasy column and it is centrifuged for about 2 min at ≧8000×g (≧10,000 rpm) to dry the RNeasy silica-gel membrane. As buffer RPE contains ethanol, special care is taken to ensure thorough drying and removal of all traces of ethanol.

9) The RNeasy column is relocated to a new 1.5 ml collection tube (supplied). An appropriate amount (30-50 μl, for example) of RNase-free water is gently pipetted directly onto the center of the RNeasy silica-gel membrane and centrifuged for 1 min at ≧8000×g (≧10,000 rpm) to elute.

10) The RNA content is then quantified by measuring the optical density of a 100× dilution and applying the following formula from Beer's law:

A₂₆₀·Dilution Factor·40=[RNA] in μg/1 ml

11) The RNA is then qualitatively assessed by denaturing agarose gel electrophoresis with ethidium bromide staining according to methods known in the art.

### RNA Labeling (Modified Eberwine Process, Van Gelder et al., 1990)

1) Synthesis of First Strand cDNA

1.1 Prepare working solution of Bacterial Control mRNAs

1.2 Prepare each total RNA sample for manual target preparation (exemplary proportions are given below):

| | | |
|---|---|---|
| 0.2-2 | μg | total RNA |
| X | μl | working solution of bacteria control mRNAs |
| | | (1 μl per 1 μg input total RNA, dilute as required) |
| 1 | μl | T7 oligo (dT) primer |
| Y | μl | nuclease-free water |
| 12 | μl | Final Volume |

1.3 Incubate approximately 10 min in 70° C. water bath; immediately place tube on ice until cool.

1.4 Centrifuge for 5 s to collect sample at the bottom of the tube; return tube to ice.

1.5 Keep the tube on ice and add the following reagents to the total volume (12 μl, in the above example) of total RNA/control mRNA/primer mix from above:

| | |
|---|---|
| 2 μl | 10x first strand buffer |
| 4 μl | 5 mM dNTP mix |
| 1 μl | RNase inhibitor |
| 1 μl | reverse transcriptase |
| 20 μl | Final volume |

1.6 Incubate 2 h in a 42° C. water bath

1.7 Centrifuge for 5 s to collect sample at the bottom of the tube.

2) Synthesis of Second Strand cDNA

2.1 Prepare the following second strand cDNA synthesis mix for each sample from step 2:

| Approximately 20 | μl | First strand cDNA reaction from step 1.7 |
|---|---|---|
| 63 | μl | Nuclease-free water |
| 10 | μl | 10x second strand buffer |
| 4 | μl | 5 mM dNTP mix |
| 2 | μl | DNA polymerase mix |
| 1 | μl | RNaseH |
| 100 | μl | Final Volume |

2.2 Gently flick tube to mix then centrifuge at ≧10 000×g to combine the reactants. Incubate 2 h at 16° C. in an incubator.

2.3 Centrifuge 5 s at maximum speed to collect sample at the bottom of the tube. Mix gently and place the tube on ice. Proceed directly to step 4 or store overnight at −20° C.

3) Purification of Double-Stranded cDNA (Qiagen, Boom et al., 1990)

3.1 Add approximately 500 μl buffer PB to the cDNA from step 2.3 and mix gently by pipetting up and down.

3.2 Place a QIAquick spin column into a 2 ml collection tube.

3.3 Transfer the cDNA buffer PB solution to the QIAquick spin column.

3.4 Centrifuge the spin column at ≧10 000×g for 30-60 s.

3.5 Discard the flow-through. To wash the column, add 700 μl buffer PE to the column and centrifuge at ≧10 000×g.

3.6 Discard the flow-through. To dry the column, place the QIAquick column into a new 2-ml collection tube and centrifuge at ≧10 000×g for 1 min.

3.7 Place the QIAquick column into a clean 1.5-ml microcentrifuge tube.

3.8 To elute the cDNA, add approximately 30 μl of EB buffer to the center of the QIAquick membrane. Let the column stand for 1 min, then centrifuge for 1 min at ≧10 000×g. Repeat once to generate a total of approximately 60 μl eluate. 3.9 Dry the cDNA solution in a SpeedVac concentrator under medium heat (approximately 1 h).

4) Synthesis of cRNA by In Vitro Transcription (IVT) Using T7 RNA Polymerase (All Measurements are Approximate)

4.1 Make a resuspension solution by placing the following components in a new RNase-free microcentrifuge tube (approximate volumes):

| | |
|---|---|
| 9.5 μl | Nuclease-free water |
| 4.0 μl | 10X T7 reaction buffer |
| 13.5 μl | Total Volume |

4.2 Resuspend each cDNA pellet from step 3.9 with 13.5 μl of the above resuspension solution. Pipette up and down to ensure resuspension of the pellet(s).

4.3 Make the IVT mixture by adding the following in another RNase-free microcentrifuge tube (volumes are approximate):

| | |
|---|---|
| 4.0 μL | T7 ATP solution |
| 4.0 μL | T7 GTP solution |
| 4.0 μL | T7 CTP solution |
| 3.0 μL | T7 UTP solution |
| 7.5 μl | mM biotin-11-UTP |
| 4.0 μl | 10X T7 enzyme mix |
| 26.5 μl | Final Volume |

4.4 Mix the components of the IVT mixture by briefly vortexing the tube. Centrifuge the tube for 5 s at ≧10 000×g. Transfer this reaction mixture (26.5 μl) into the tube of resuspended cDNA from step 4.2, and gently pipette up and down to ensure complete mixing.

4.5 Incubate the reaction for 14 h in an air incubator at 37° C. (Water baths and open heat blocks are not recommended due to condensation on the lid of the microcentrifuge tube)

5) Recovery of Biotin Labelled cRNA (Qiagen, Boom et al., 1990) (All Measurements are Approximate)

5.1 Prepare working solutions of the RLT and RPE buffers.

5.2 Adjust the IVT reaction volume to approximately 100 μl by adding an appropriate volume (for example, 60 μl) of nuclease-free water.

5.3 Add 350 μl buffer RLT to the sample and mix thoroughly by pipetting up and down.

5.4 Add 250 μl of 100% ethanol to the reaction sample and mix well by pipetting up and down. Do not centrifuge.

5.5 Apply the sample (700 μl) to an RNeasy spin column in a collection tube. Centrifuge for 15 s at ≧8000×g.

5.6 Transfer the RNeasy column into a new 2-ml collection tube (supplied). Add 500 μl of buffer RPE to column and centrifuge for 15 s at ≧8000×g. Discard flow-through and reuse the collection tube. Repeat this wash step once.

5.7 Place the RNeasy spin column into a new 2 μl collection tube and centrifuge at ≧8000×g for 2 min to dry the membrane.

5.8 Transfer the RNeasy column into a new 1.5-ml collection tube (supplied), and pipette 50 μl of nuclease-free water directly onto the RNeasy membrane without touching the membrane.

5.9 Incubate at ambient temperature for 10 minutes. Elute the cRNA by centrifugation at ≧8000×g for 1 min. Do not remove the column.

5.10 Pipette another 50 μl of nuclease-free water directly onto the same RNeasy membrane.

5.11 Incubate at ambient temperature for 10 min. Elute the cRNA (into the same tube used in step 5.8) by centrifugation at ≧8000×g for 1 min.

5.12 Remove the column. Mix the solution by flicking the tube.

5.13 Store the cRNA at −70° C.

6) Assessment of cRNA Concentration, Yield and Quality

The UV spectrophotometric quantitation method is described. Other methods of quality analysis involve either denaturing gel-electrophoresis or Agilent 2100 Bioanalyzer.

6.1 Prepare a appropriate dilution (for example, 1:50) for each sample of cRNA in nuclease-free water:

| | | |
|---|---|---|
| 2 | μl | cRNA |
| 98 | μl | nuclease-free water |
| 100 | μl | Final Volume |

6.2 Vortex the mixture and centrifuge for 5 s at ≧10 000×g to collect all fluid at the bottom of the tube.

6.3 Transfer the cRNA dilution to a 100-μl quartz cuvette (1-cm pathlength) and measure UV absorbance at 260 nm. If the A₂₆₀ values are less than 0.15, prepare a 1:20 dilution of the cRNA and measure its UV absorbance at 260 nm. For accurate concentration determination, the dilution must yield an A260 in the linear range of approximately 0.15-0.95. If the initial reading with 1:50 dilution is not in the linear range, prepare another sample with the appropriate dilution change.

6.4 Calculate the cRNA concentration:

1A₂₆₀ unit=40 μg/ml (1-cm pathlength cuvette):

Concentration in μg/μl=A₂₆₀×dilution factor×40 μg/ml×0.001 ml/μl

6.5 The A₂₆₀:A₂₈₀ ratio is sensitive to pH. Thus, to accurately determine the purity of the sample, it can be buffered. Transfer the diluted cRNA from the cuvette to a new microcentrifuge tube and add 11.1 μl of 0.1 M Tris-HCl, pH 7.6. Vortex the mixture.

6.6 Transfer the Tris-diluted cRNA (˜100 μl) to a quartz cuvette and measure UV absorbance at 260 nm and 280 nm. The A₂₆₀:A₂₈₀ ratio is a measure of sample purity and should be within 1.8-2.1.

1.1 For each array to be loaded, bring approximately 10 μg of cRNA (from step 6.6) to a final volume of about 20 μl with nuclease-free water in a thin walled microcentrifuge tube.

1.2 Add 5 μl of 5× buffer for each microarray. Place tube in a thermal cycler and heat for 20 min at 94° C. using the heated lid feature.

1.3 Cool to 0° C. in the thermal cycler for at least 5 min.

### Hybridization and Washing:

1) Fragmentation of cRNA in Preparation for Hybridisation to the Array

1.1 For each microarray to be loaded, bring 10 μg of cRNA (from step 6.6) to a final volume of 20 μl with nuclease-free water in a thin walled microcentrifuge tube.

1.2 Add 5 μl of 5× fragmentation buffer for each microarray. Place tube in a thermal cycler and heat for 20 min at 94° C. using the heated lid feature.

1.3 Cool to 0° C. in the thermal cycler for at least 5 min.

### 2) Preparation of Hybridization Reaction Mixtures

2.1 Set the temperature of the shaker-incubator to 37° C. for hybridization. Assemble microarray tray posts from the CodeLink Shaker Kit onto the incubator platform.

2.2 For each microarray to be processed, prepare 260 μl of hybridization solution containing 10 μg of fragmented target cRNA in a 1.5-microcentrifuge tube:

| | | |
|---|---|---|
| 78 | μl | hybridization buffer component A |
| 130 | μl | hybridization buffer component B |
| 27 | μl | nuclease-free water |
| 25 | μl | fragmented cRNA (from section 1) |
| 260 | μl | total volume |

2.3 Vortex the solution for 5 s at maximum speed. Incubate the hybridization solution at 90° C. for 5 min to denature the cRNA.

2.4 Cool the tube(s) on ice for at least 5 and no more than 30 min. Load all microarrays within 30 min of denaturing the cRNA.

3) Loading of Reaction Mixtures into Microarray Chambers

3.1 Set a slide shaker tray on a level surface. Place the microarrays into the shaker tray with the input/output ports facing up. Load microarrays in sets of 12 or less.

3.2 Vortex the hybridization reaction mixture for 5 s at maximum speed. Centrifuge briefly to gather the liquid at the bottom of the tube. Place the tube back on ice.

3.3 For each microarray, draw 250 μl into a 1-ml wide-bore pipette tip and slowly inject the entire sample into the chamber without using the blowout feature of the pipettor. Discard any excess target mix remaining in the pipette tip. Aspirate any excess fluid surrounding the outside of the port with a pipette tip and discard. Use a lint-free wipe to blot residual fluid from around the port, taking care not to actually touch the port.

3.4 After loading up to 12 microarrays, seal the Flex Chamber ports using sealing strips and port sealing tool. Do not touch the Flex Chamber or directly depress the port.

### 4) Hybridization

4.1 Align the 12-slide shaker tray notches with the front and back posts fixed to the shaker-incubator platform to place the loaded shaker tray into the shaker-incubator. The Flex Chamber should be facing up.

4.2 Set the shaker speed to 300 rpm and incubate slides for 18-24 h at 37° C. It is critical that arrays used in any form of comparison are hybridized for the same amount of time within the given range.

4.3 To prepare for the next step, fill a large reagent reservoir with 240 ml of filtered 0.75×TNT buffer. Cover the reservoir and incubate in a 46° C. water bath overnight.

### 5) Post-Hybridization Wash

5.1 Fill each slot in the medium reagent reservoir with 13 ml of filtered 0.75×TNT. Place the microarray rack into the reservoir. Leave at ambient temperature.

5.2 Remove the 12-slide shaker tray from the shaker incubator, and place on a level surface at ambient temperature. Process only 12 slides at a time per person. Leave the trays in the shaker until ready to process.

5.3 Place the first microarray to be processed into the Flex Chamber removal tool.

5.4 Remove the Flex Chamber by lifting the tab and slowly pulling it back at a 60° angle without creasing the Flex Chamber.

5.5 Place the microarray into a slot of the microarray rack, which was placed to a medium reagent reservoir containing 0.75×TNT in step 5.1. Use the microarray position tool, tooth-side down, to ensure the microarrays are properly seated.

5.6 To avoid potential cross-contamination, rinse the surface of the Flex Chamber removal tool with approximately 5 ml of ambient temperature 0.75×TNT buffer dispensed from a squirt bottle. Keep the medium reagent reservoir at ambient temperature until all microarrays have been processed.

5.7 Repeat steps 5.3-5.6 for each hybridized microarray.

5.8 Transfer the microarray rack with microarrays from the medium reagent reservoir into the pre-warmed, 0.75×TNT-filled large reagent reservoir from step 4.3. Replace the lid on the large reagent reservoir and then on the water bath. Incubate at 46° C. for exactly 1 h; longer incubation time may significantly reduce signal intensities.

Detection with Streptavidin-Dye Conjugate

6.1 Fill each slot in the small reagent reservoir with 3.4 ml of Cy5-Streptavidin working solution. Leave at ambient temperature. Cover the small reagent reservoir with its lid to prevent photobleaching of the fluorophore by ambient light.

6.2 Remove the microarray rack with microarrays from the large reagent reservoir at 46° C., and place into the small reagent reservoir containing the Cy5-Streptavidin working solution. Cover with the lid and incubate microarrays at ambient temperature for 30 min.

6.3 During incubation, prepare for the wash steps by filling four large reagent reservoirs each with 240 ml of ambient temperature 1×TNT buffer.

6.4 After the 30 min incubation, remove the microarray rack with microarrays from the staining solution and place into one large reagent reservoir containing 1×TNT buffer (prepared in 6.3). Do not drain the solution from the microarrays. Incubate at ambient temperature for 5 min, covered from light.

6.5 Remove the microarray rack from the first large reagent reservoir with 1×TNT buffer and place into a second large reagent reservoir containing 1×TNT buffer. Again, do not attempt to drain the solution from the microarrays. Incubate at ambient temperature for 5 min, covered from light. Repeat this step with two additional large reagent reservoirs containing fresh 1×TNT buffer for a third and fourth wash.

6.6 During the third wash, thoroughly rinse a large reagent reservoir with distilled water and dry it. Completely fill this reservoir with the final rinse 0.1×SSC/0.05% Tween 20 solution.

6.7 Transfer the microarray rack into the large reagent reservoir completely filled with 0.1×SSC/0.05% Tween 20 at ambient temperature. Incubate the slides for 30 seconds while continually agitating mildly up and down.

6.8 Remove the microarray rack from the large reagent reservoir and blot the bottom edge of the microarrays briefly on an absorbent paper towel. Place the microarray rack in a clean, dry medium reagent reservoir. Dry the microarrays by centrifugation in the Qiagen Sigma 4-15 C centrifuge with corresponding bucket rotor (2×96-well plate) or similar system using following settings:

### Speed: 2000 rpm (644×g)

### Acceleration: 9

### Deceleration: 9

### Time: 3 min

6.9 Use the microarray removal tool to easily remove the microarrays from the microarray rack, and place the dry microarrays into a light-protected slide box until they are scanned. Microarrays should be scanned within two days of assay completion.

6.10 Repeatedly rinse all reservoirs with deionized water to clean, and invert to dry.

6.11 Wash the rack with Alconox™ soap, scrubbing between the rails with a pipe cleaner style brush. Rinse thoroughly with deionized water to remove residual soap. Air-dry the rack.

### 7) Microarray Scanning and Analysis

7.1 Turn the scanner on 15 min prior to use.

7.2 Slide the cover to the left to expose the slide holder.

7.3 Lift the latch of the slide holder and lift the upper clip.

7.4 Wearing latex gloves, load the microarray into the tray with the label side down and closest to the front of the scanner.

7.5 Pull the clip on the left of the slide out and let the microarray fall into place. Release the clip to put pressure against the microarray.

7.6 Grab microarray by the edges and move the microarray toward you.

7.7 Lower upper clip and press down on the latch until it clicks.

7.8 Slide the cover to the right to cover the slide holder.

7.9 Open the array analysis software program and select the following settings:

### Wavelength: 635 nm

PMT voltage: 600 V
Laser power: 100%
Pixel size: 10 μm
Focus position: 0 μm

7.10 The hematological cancer array can be scanned according to the standard protocol.

7.11 Enter the microarray serial number and click Next. The Experiment and Scan Information interface is displayed.

7.12 Type in the project name, experiment name, and sample name. The username is automatically captured. When opening this interface for the first time, a message box may ask whether to allow an ActiveX interaction to proceed.

7.13 Select a setting (.gps) file. If a settings file was previously selected, the name and path are displayed under Current Settings File. To select a new file, click Browse under Select New Settings File. The values for project name, experiment name, user name, and settings file that were entered for a previous microarray are retained but may be changed.

7.14 In the Load and Scan Slide screen, the standard TIF file name for the current microarray is displayed. If desired, the name may be changed.

7.15 Click Browse to select the image path or the location where the image files will be stored. If a Security Alert message box is displayed.

7.16 Click Scan Slide. The Image tab will display, and the instrument will perform the scan.

7.17 When complete, the view will return to the Report tab.

7.18 Click Save Image to save the scanned image at the appropriate file location.

7.19 Slide the cover to the left and remove the microarray.

7.20 To scan the next microarray, click New Slide and enter the serial number for the next microarray. The setting information previously entered will be retained.

7.21 Analyze the image from each microarray using the software.

### Example 14

### Selection of Additional Genes for HCP Sets Relating to Lymphoma or Leukemia

Genes whose expression pattern is indicative of one or more features of a hematological cancer selected from the group of lymphoma and leukemia were selected for inclusion into HCP sets as follows. Initial gene selection was carried out by consulting numerous publications to identify genes that have been shown to hold diagnostic or prognostic potential for lymphoma and/or leukemia. A list of publications consulted is found below:
Staudt L M. Molecular diagnosis of the hematologic cancers. N Engl J Med. 2003; 348(18): 1777-85. [Erratum: N Engl J Med. 2003; 348(25):2588.]
Diehl V, Thomas R K, Re D. Part II: Hodgkin's lymphoma—diagnosis and treatment. Lancet Oncol. 2004; 5(1):19-26.
Soukup J, Krskova L, Mrhalova M, et al. Large-cell diffuse B-cell lymphoma: heterogenous origin and prognosis from the aspect of modern diagnosis. Cas Lek Cesk. 2003; 142(7):417-2
Guermazi A, Brice P, Hennequin C, et al. Lymphography: an old technique retains its usefulness. Radiographics. 2003; 23(6):1541-58; discussion 1559-60.
Alizadeh A A, Eisen M B, Davis R E et al. Distinct types of diffuse large B-cell lymphoma identified by gene expression profiling. Nature. 2000; 403(6769):503-11.
Harris N L, Jaffe E S, Diebold J et al. Lymphoma classification—from controversy to consensus: the R.E.A.L. and WHO Classification of lymphoid neoplasms. Ann Oncol. 2000; 11 Suppl 1:3-10.
Rosenwald A, Wright G, Chan W C et al. The use of molecular profiling to predict survival after chemotherapy for diffuse large-B-cell lymphoma. N Engl J Med. 2002; 346(25): 1937-47.
Rosenwald A, Wright G, Leroy K. et al. Molecular diagnosis of primary mediastinal B cell lymphoma identifies a clinically favorable subgroup of diffuse large B cell lymphoma related to Hodgkin lymphoma. J Exp Med. 2003; 198(6):851-62.
Wright G, Tan B, Rosenwald A, et al. A gene expression-based method to diagnose clinically distinct subgroups of diffuse large B cell lymphoma Proc Natl Acad Sci USA. 2003; 100(17):9991-6
Savage K J, Monti S, Kutok J L, et al. The molecular signature of mediastinal large B-cell lymphoma differs from that of other diffuse large B-cell lymphomas and shares features with classical Hodgkin lymphoma. Blood. 2003; 102(12):3871-9
Shipp M A, Ross K N, Tamayo P et al. Diffuse large B-cell lymphoma outcome prediction by gene-expression profiling and supervised machine learning. Nat Med. 2002 January; 8(1):68-74.
Pan Z, Shen Y, Du C, et al. Two newly characterized germinal center B-cell-associated genes, GCET1 and GCET2, have differential expression in normal and neoplastic B cells. Am J Pathol. 2003; 163(1):135-44.
Nishiu M, Yanagawa R, Nakatsuka S, et al. Microarray analysis of gene-expression profiles in diffuse large B-cell lymphoma: identification of genes related to disease progression. Jpn J Cancer Res. 2002; 93(8):894-901.
Lossos I S, Alizadeh A A, Diehn M, et al. Transformation of follicular lymphoma to diffuse large-cell lymphoma: alternative patterns with increased or decreased expression of c-myc and its regulated genes. Proc Natl Acad Sci USA. 2002; 99(13):8886-91.
de Vos S, Hofmann W K, Grogan T M, et al. Gene expression profile of serial samples of transformed B-cell lymphomas. Lab Invest. 2003; 83(2):271-85.
Kossakowska A E, Urbanski S J, Watson A, et al. Patterns of expression of metalloproteinases and their inhibitors in human malignant lymphomas. Oncol Res. 1993; 5(1):19-28.
Husson H, Carideo E G, Neuberg D, et al. Gene expression profiling of follicular lymphoma and normal germinal center B cells using cDNA arrays. Blood. 2002; 99(1):282-9.
Lestou V S, Gascoyne R D, Sehn L, et al. Multicolour fluorescence in situ hybridization analysis of t(14;18)-positive follicular lymphoma and correlation with gene expression data and clinical outcome. Br J Haematol. 2003; 122(5):745-59.
Maesako Y, Uchiyama T, Ohno H. Comparison of gene expression profiles of lymphoma cell lines from transformed follicular lymphoma, Burkitt's lymphoma and de novo diffuse large B-cell lymphoma. Cancer Sci. 2003; 94(9):774-81.
Ghia P, Boussiotis V A, Schultze J L, et al. Unbalanced expression of bcl-2 family proteins in follicular lymphoma: contribution of CD40 signaling in promoting survival. Blood. 1998; 91(1):244-51.
Akasaka T, Lossos I S, Levy R. BCL6 gene translocation in follicular lymphoma: a harbinger of eventual transformation to diffuse aggressive lymphoma. Blood. 2003; 102(4):1443-8.
Elenitoba-Johnson K S, Gascoyne R D, Lim M S, et al. Homozygous deletions at chromosome 9p21 involving p16 and p15 are associated with histologic progression in follicle center lymphoma. Blood. 1998; 91(12):4677-85.
Rosenwald A, Alizadeh A A, Widhopf G, et al. Relation of gene expression phenotype to immunoglobulin mutation genotype in B cell chronic lymphocytic leukemia. J Exp Med. 2001; 194(11):1639-47.
Hamblin T J, Davis Z, Gardiner A, et al. Unmutated Ig V(H) genes are associated with a more aggressive form of chronic lymphocytic leukemia. Blood. 1999; 94(6):1848-54.
Damle R N, Wasil T, Fais F, et al. Ig V gene mutation status and CD38 expression as novel prognostic indicators in chronic lymphocytic leukemia. Blood. 1999; 94(6): 1840-7
Wiestner A, Rosenwald A, Barry T S, et al. ZAP-70 expression identifies a chronic lymphocytic leukemia subtype with unmutated immunoglobulin genes, inferior clinical outcome, and distinct gene expression profile. Blood. 2003; 101(12):4944-51.
Nagy B, Ferrer A, Larramendy M L, et al. Lymphotoxin beta expression is high in chronic lymphocytic leukemia but low in small lymphocytic lymphoma: a quantitative real-time reverse transcriptase polymerase chain reaction analysis. Haematologica. 2003; 88(6):654-8.
Cro L, Guffanti A, Colombi M, et al. Diagnostic role and prognostic significance of a simplified immunophenotypic classification of mature B cell chronic lymphoid leukemias. Leukemia. 2003; 17(1):125-32.
Thieblemont C, Chettab K, Felman P, et al. Identification and validation of seven genes, as potential markers, for the differential diagnosis of small B cell lymphomas (small lymphocytic lymphoma, marginal zone B cell lymphoma and mantle cell lymphoma) by cDNA macroarrays analysis. Leukemia. 2002; 16(11):2326-9
Korz C, Pscherer A, Benner A, et al. Evidence for distinct pathomechanisms in B-cell chronic lymphocytic leukemia and mantle cell lymphoma by quantitative expression analysis of cell cycle and apoptosis-associated genes. Blood. 2002; 99(12):4554-61.
Robetorye R S, Bohling S D, Morgan J W, et al. Microarray analysis of B-cell lymphoma cell lines with the t(14;18). J Mol Diagn. 2002; 4(3):123-36.
Martinez N, Camacho F I, Algara P, et al. The molecular signature of mantle cell lymphoma reveals multiple signals favoring cell survival. Cancer Res. 2003; 63(23):8226-32.
Kobayashi T, Yamaguchi M, Kim S, et al. Microarray reveals differences in both tumors and vascular specific gene expression in de novo CD5+ and CD5− diffuse large B-cell lymphomas. Cancer Res. 2003; 63(1):60-6.
Devilard E, Bertucci F, Trempat P, et al. Gene expression profiling defines molecular subtypes of classical Hodgkin's disease. Oncogene. 2002; 21(19):3095-102.
Kuppers R, Klein U, Schwering I, et al. Identification of Hodgkin and Reed-Sternberg cell-specific genes by gene expression profiling. J Clin Invest. 2003; 111(4):529-37.
Thorns C, Gaiser T, Lange K, et al. cDNA arrays: gene expression profiles of Hodgkin's disease and anaplastic large cell lymphoma cell lines. Pathol Int. 2002; 52(9):578-85.
Villalva C, Trempat P, Greenland C, et al. Isolation of differentially expressed genes in NPM-ALK-positive anaplastic large cell lymphoma. Br J Haematol. 2002; 118(3):791-8.
Wellmann A, Thieblemont C, Pittaluga S, et al. Detection of differentially expressed genes in lymphomas using cDNA arrays: identification of clusterin as a new diagnostic marker for anaplastic large-cell lymphomas. Blood. 2000 Jul. 15; 96(2):398-404.
Nishikori M, Maesako Y, Ueda C, et al. High-level expression of BCL3 differentiates t(2;5)(p23;q35)-positive anaplastic large cell lymphoma from Hodgkin disease. Blood. 2003; 101(7):2789-96.
American Society of Hematology. Newly identified genes may help predict outcome in childhood leukemia. August 2003 Press Release.
Golub T R, Slonim D K, Tamayo P, Huard C, Gaasenbeek M, Mesirov J P, Coller H, Loh M L, Downing J R, Caligiuri M A, Bloomfield C D and E S Lander. Molecular classification of cancer: class discovery and class prediction by gene expression monitoring. Science 1999 Oct. 15; 286(5439): 531-537
Bhojwani D, Min D J, Raetz E and W L Carroll. NEW APPROACHES TO RISK-ADAPTED THERAPY FOR CHILDHOOD ACUTE LYMPHOBLASTIC LEUKEMIA. Hematology 2003: 102-131
National Cancer Institute Website. http://www.cancer.gov/cancerinformation/cancertype/leukemia
Downing J R. Expression Profiling In Acute Leukemia. Hematology 2003:102-131
Brunet J P, Tamayo P, Golub T R and J P Mesirov. Metagenes and molecular pattern discovery using matrix factorization. Proc Natl Acad Sci USA. 2004 Mar. 23; 101(12): 4164-4169
Ferrando A A, Neuberg D S, Staunton J, Loh M L, Huard C, Raimondi S C, Behm F G, Pui C H, Downing J R, Gilliland D G, Lander E S, Golub T R and A T Look. Gene expression signatures define novel oncogenic pathways in T cell acute lymphoblastic leukemia. Cancer Cell 2002 February; 1(1): 75-87
Reed J C. Pediatric Acute Lymphoblastic Leukemia. Hematology 2003: 102-131
Sanz L, Garcia-Marco J A, Casanova B, de La Fuente M T, Garcia-Gila M, Garcia-Pardo A and A Silva. Bcl-2 family gene modulation during spontaneous apoptosis of B-chronic lymphocytic leukemia cells. Biochem Biophys Res Commun. 2004 Mar. 12; 315(3): 562-567
Mamani-Matsuda M, Moynet D, Molimard M, Ferry-Dumazet H, Marit G, Reiffers J and M D Mossalayi. Long-acting beta2-adrenergic formoterol and salmeterol induce the apoptosis of B-chronic lymphocytic leukaemia cells. Br J Haematol. 2004 January; 124(2): 141-150
Bueso-Ramos C E, Rocha F C, Shishodia S, Medeiros L J, Kantarjian H M, Vadhan-Raj S, Estrov Z, Smith T L, Nguyeni M H and B B Aggarwal. Expression of constitutively active nuclear-kappa B RelA transcription factor in blasts of acute myeloid leukemia. Hum Pathol. 2004 February; 35(2): 246-253
Floros K V, Thomadaki H, Lallas G, Katsaros N, Talieri M and A Scorilas. Cisplatin-induced apoptosis in HL-60 human promyelocytic leukemia cells: differential expression of BCL2 and novel apoptosis-related gene BCL2L12. Ann N Y Acad Sci. 2003 December; 1010: 153-158
Armstrong S A, Staunton J E, Silverman L B, Pieters R, den Boer M L, Minden M D, Sallan S E, Lander E S, Golub T R and S J Korsmeyer. MLL translocations specify a distinct gene expression profile that distinguishes a unique leukemia. Nature Genetics 2002 January; 30(1): 41-47
Bullinger L, Dohner K, Bair E, Frohling S, Schlenk R F, Tibshirani R, Dohner H and JR Pollack. Use of Gene-Expression Profiling to Identify Prognostic Subclasses in Adult Acute Myeloid Leukemia. 2004 Apr. 15; 350(16): 1605-1616
Valk P J, Verhaak R G, Beijen M A, Erpelinck C A, Barjesteh van Waalwijk van Doom-Khosrovani S, Boer J M, Beverloo H B, Moorhouse M J, van der Spek P J, Lowenberg B and R Delwel. Prognostically useful gene-expression profiles in acute myeloid leukemia. N Engl J Med 2004 Apr. 15; 350(16): 1617-1628
Carroll W L, Bhojwani D, Min D J, Raetz E, Relling M, Davies S, Downing J R, Willman C L and J C Reed. Pediatric Acute Lymphoblastic Leukemia. Hematology 2003: 102-131
Rozovskaia T, Ravid-Amir O, Tillib S, Getz G, Feinstein E, Agrawal H and Nagler. Expression profiles of acute lymphoblastic and myeloblastic leukemias with ALL-1 rearrangements. Proc Natl Acad Sci USA. 2003 Jun. 24; 100(13): 7853-7858
Ferrando A A, Neuberg D S, Dodge R K, Paietta E, Larson R A, Wiernik P H, Rowe J M, Caligiuri M A, Bloomfield C D and A T Look. Prognostic importance of TLX1 (HOX11) oncogene expression in adults with T-cell acute lymphoblastic leukaemia. Lancet. 2004 Feb. 14; 363(9408): 535-536.
Galimberti S, Guerrini F, Carulli G, Fazzi R, Palumbo G A, Morabito F and M Petrini. Significant co-expression of WT1 and MDR1 genes in acute myeloid leukemia patients at diagnosis. Eur J Haematol. 2004 January; 72(1): 45-51
Willman C L. Pediatric Acute Lymphoblastic Leukemia. Hematology 2003: 102-131 Ross M E, Zhou X, Song G, Shurtleff S A, Girtman K, Williams W K, Liu H C, Mahfouz R, Raimondi S C, Lenny N, Patel A and J R Downing. Classification of pediatric acute lymphoblastic leukemia by gene expression profiling. Blood 2003 Oct. 15; 102(8): 2951-2959
Moos P J, Raetz E A, Carlson M A, Szabo A, Smith F E, Willman C, Wei Q, Hunger S P and W L Carroll. Identification of gene expression profiles that segregate patients with childhood leukemia. Clin Cancer Res. 2002 October; 8(10): 3118-3130
Health A to Z website www.healthatoz.com/healthatoz/Atoz/ency/leukemias_chronic.jsp
BC Cancer Agency website
American Cancer Society website
Mackey J R, Galmarini C M, Graham K A, Joy A A, Delmer A, Dabbaugh L, Glubrecht D, Jewell L D, Lai R, Lang T, Young J D, Merle-Beral H, Binet J L, Cass C E and C Dumontet. Quantitative analysis of nucleoside transporter and metabolism gene expression in chronic lymphocytic leukemia (CLL)-identification of fludarabine-sensitive and insensitive populations. Blood 2005 Jan. 15; 105(2): 767-774
Vallat L, Magdelenat H, Merle-Beral H, Masdehors P, Potocki de Montalk G, Davi F, Kruhoffer M, Sabatier L, Orntoft T F and J Delic. The resistance of B-CLL cells to DNA damage-induced apoptosis defined by DNA microarrays. Blood 2003 Jun. 1; 101(11): 4598-4606
Chiaretti S, Li X, Gentleman R, Vitale A, Vignetti M, Mandelli F, Ritz J and R Foa. Gene expression profile of adult T-cell acute lymphocytic leukemia identifies distinct subsets of patients with different response to therapy and survival. Blood. 2004 Apr. 1; 103(7): 2771-2778
Baldus C D, Liyanarachchi S, Mrozek K, Auer H, Tanner S M, Guimond M, Ruppert A S, Mohamed N, Davuluri R V, Caligiuri M A, Bloomfield C D and A de la Chapelle. Acute myeloid leukemia with complex karyotypes and abnormal chromosome 21: Amplification discloses overexpression of APP, ETS2, and ERG genes. Proc Natl Acad Sci USA. 2004 Mar. 16; 101(11): 3915-3920
Cario G, Stanulla M, Fine B M, Teuffel O, V Neuhoff N, Schrauder A, Flohr T, Schafer B W, Bartram C R, Welte K, Schlegelberger B and M Schrappe. Distinct gene expression profiles determine molecular treatment response in childhood acute lymphoblastic leukemia. Blood. 2004 Sep. 23; [Epub ahead of print]
Dohner H, Stilgenbauer S, Benner A, Leupolt E, Krober A, Bullinger L, Dohner K, Bentz M and P Lichter. Genomic aberrations and survival in chronic lymphocytic leukemia. N Engl J Med. 2000 Dec. 28; 343(26): 1910-1916
Ferrer A, Ollila J, Tobin G, Nagy B, Thunberg U, Aalto Y, Vihinen M, Vilpo J, Rosenquist R and S Knuutila. Different gene expression in immunoglobulin-mutated and immunoglobulin-unmutated forms of chronic lymphocytic leukemia. Cancer Genet Cytogenet. 2004 August; 153(1): 69-72
Ghobrial I M, Bone N D, Stenson M J, Novak A, Hedin K E, Kay N E and S M Ansell. Expression of the chemokine receptors CXCR4 and CCR7 and disease progression in B-cell chronic lymphocytic leukemia/small lymphocytic lymphoma. Mayo Clin Proc. 2004 March; 79(3): 318-25
Galimberti S, Guerrini F, Palumbo G A, Consoli U, Fazzi R, Morabito F, Santini V and M Petrini. Evaluation of BCRP and MDR-1 co-expression by quantitative molecular assessment in AML patients. Leuk Res. 2004 April; 28(4): 367-372
Gery S, Park D J, Vuong P T, Chih D Y, Lemp N and H P Koeffier. Expression of C/EBP Homologous Protein (CHOP) is Regulated by Retinoic Acid, and CHOP Negatively Regulates Myeloid Target Genes.
Graux C, Cools J, Melotte C, Quentmeier H, Ferrando A, Levine R, Vermeesch J R, Stul M, Dutta B, Boeckx N, Bosly A, Heimann P, Uyttebroeck A, Mentens N, Somers R, MacLeod R A, Drexler H G, Look A T, Gilliland D G, Michaux L, Vandenberghe P, Wlodarska I, Marynen P and A Hagemeijer. Fusion of NUP214 to ABL1 on amplified episomes in T-cell acute lymphoblastic leukemia. Nat Genet. 2004 October; 36(10): 1084-9
Gruszka-Westwood A M, Horsley S W, Martinez-Ramirez A, Harrison C J, Kempski H, Moorman A V, Ross F M, Griffiths M, Greaves M F and L Kearney. Comparative expressed sequence hybridization studies of high-hyperdiploid childhood acute lymphoblastic leukemia. Genes Chromosomes Cancer. 2004 November; 41(3): 191-202
Holleman A, Cheok M H, den Boer M L, Yang W, Veerman A Y, Kazemier K M, Pei D, Cheng C, Pui C H, Relling M V, Janka-Schaub G E, Pieters R and W E Evans. Gene-expression patterns in drug-resistant acute lymphoblastic leukemia cells and response to treatment. N Engl J Med. 2004 Aug. 5; 351(6): 533-542
Jelinek D F, Tschumper R C, Stolovitzky G A, Iturria S J, Tu Y, Lepre J, Shah N and N E Kay. Identification of a global gene expression signature of B-chronic lymphocytic leukemia. Mol Cancer Res. 2003 March; 1(5): 346-361
Kawagoe H, Potter M, Ellis J and G C Grosveld. TEL2, an ETS factor expressed in human leukemia, regulates monocytic differentiation of U937 Cells and blocks the inhibitory effect of TELL on ras-induced cellular transformation. Cancer Res. 2004 Sep. 1; 64(17): 6091-6100
Leupin N, Luthi A, Novak U, Grob T J, Hugli B, Graber H, Fey M F and A Tobler. P73 status in B-cell chronic lymphocytic leukaemia. Leuk Lymphoma 2004 June; 45(6): 1205-1207
Lin J T, Wu M S, Wang W S, Yen C C, Chiou T J, Liu J H, Yang M H, Chao T C, Chou S C and P M Chen. All-trans retinoid acid increases Notch1 transcript expression in acute promyelocytic leukemia. Adv Ther. 2003 November-December; 20(6): 337-43
Lu D, Zhao Y, Tawatao R, Cottam H B, Sen M, Leoni L M, Kipps T J, Corr M and D A Carson. Activation of the Wnt signaling pathway in chronic lymphocytic leukemia. Proc Natl Acad Sci USA. 2004 Mar. 2; 101(9): 3118
Mackey J R, Galmarini C M, Graham K A, Joy A A, Delmer A, Dabbaugh L, Glubrecht D, Jewell L D, Lai R, Lang T, Young J D, Merle-Beral H, Binet J L, Cass C E and C Dumontet. Quantitative analysis of nucleoside transporter and metabolism gene expression in chronic lymphocytic leukemia (CLL)-identification of fludarabine-sensitive and insensitive populations. Blood. 2004 Sep. 28; [Epub ahead of print]
Mauvieux L, Leymarie V, Helias C, Perrusson N, Falkenrodt A, Lioure B, Lutz P and M Lessard. High incidence of Hox11L2 expression in children with T-ALL. Leukemia. 2002 December; 16(12): 2417-2422
Mayr C, Bund D, Schlee M, Moosmann A, Kofler D M, Hallek M and C M Wendtner. Fibromodulin as a novel tumor-associated antigen (TAA) in chronic lymphocytic leukemia (CLL) which allows expansion of specific CD8+ autologous T lymphocytes.
Müller-Tidow C, Metzelder S K, Buerger H, Packeisen J, Ganser A, Heil G, Kügler K, Adigüzel G, Schwäble J, Steffen B, Ludwig W D, Heinecke A, Büchner T, Berdel W E and H Serve. Expression of the p14^{ARF} tumor suppressor predicts survival in acute myeloid leukemia. Leukemia 2004 April; 18(4): 720-726
Nagy B, Ferrer A, Larramendy M L, Galimberti S, Aalto Y, Casas S, Vilpo J, Ruutu T, Vettenranta K, Franssila K and S Knuutila. Lymphotoxin beta expression is high in chronic lymphocytic leukemia but low in small lymphocytic lymphoma: a quantitative real-time reverse transcriptase polymerase chain reaction analysis. Haematologica. 2003 June; 88(6): 654-658
Nowicki M O, Pawlowski P, Fischer T, Hess G, Pawlowski T and T Skorski. Chronic myelogenous leukemia molecular signature. Oncogene. 2003 Jun. 19; 22(25): 3952-3963
Pui, C, Relling M V and J R Downing. Mechanisms of Disease: Acute Lymphoblastic Leukemia. 2004 Apr. 8; 350(15): 1535-1548
Rizzo M G, Giombini E, Diverio D, Vignetti M, Sacchi A, Testa U, Lo-Coco F and G Blandino. Analysis of p73 expression pattern in acute myeloid leukemias: lack of DeltaN-p73 expression is a frequent feature of acute promyelocytic leukemia. Leukemia. 2004 November; 18(11):1804-1809
Safley A M, Sebastian S, Collins T S, Tirado C A, Stenzel T T, Gong J Z and B K Goodman. Molecular and cytogenetic characterization of a novel translocation t(4;22) involving the breakpoint cluster region and platelet-derived growth factor receptor-alpha genes in a patient with atypical chronic myeloid leukemia. Genes Chromosomes Cancer 2004 May; 40(1): 44-50
Sarsotti E, Marugan I, Benet I, Terol M J, Sanchez-Izquierdo D, Tormo M, Rubio-Moscardo F, Martinez-Climent J A and J García-Conde. Bcl-6 mutation status provides clinically valuable information in early-stage B-cell chronic lymphocytic leukemia. Leukemia 2004 April; 18(4): 743-746
Sauerbrey A, Sell W, Steinbach D, Voigt A and F Zintl. Expression of the BCRP gene (ABCG2/MXR/ABCP) in childhood acute lymphoblastic leukaemia. Br J Haematol. 2002 July; 118(1): 147-150
Serinsoz E, Neusch M, Busche G, Wasielewski R, Kreipe H and O Bock. Aberrant expression of beta-catenin discriminates acute myeloid leukaemia from acute lymphoblastic leukaemia. Br J Haematol. 2004 August; 126(3): 313-319
Sun Y, Dong L J, Tian F, Wang S Q, Jia Z L, Huang J, Chen Z J, Li W J, Chen X L and P Zhu. Identification of Acute Leukemia-Specific Genes from Leukemia Recipient/Sibling Donor Pairs by Distinguishing Study with Oligonucleotide Microarrays. Zhongguo Shi Yan Xue Ye Xue Za Zhi 2004 August; 12(4): 450-454
Tallman M S, Lefebvre P, Baine R M, Shoji M, Cohen I, Green D, Kwaan H C, Paietta E and F R Rickles. Effects of all-trans retinoic acid or chemotherapy on the molecular regulation of systemic blood coagulation and fibrinolysis in patients with acute promyelocytic leukemia. J Thromb Haemost. 2004 August; 2(8): 1341-1350
Tsutsumi C, Ueda M, Miyazaki Y, Yamashita Y, Choi Y L, Ota J, Kaneda R, Koinuma K, Fujiwara S, Kisanuki H, Ishikawa M, Ozawa K, Tomonaga M and H Mano. DNA microarray analysis of dysplastic morphology associated with acute myeloid leukemia. Exp Hematol. 2004 September; 32(9): 828-835
van der Burg M, Poulsen T S, Hunger S P, Beverloo H B, Smit E M E, Vang-Nielsen K, Langerak A W and J J M van Dongen. Split-signal FISH for detection of chromosome aberrations in acute lymphoblastic leukemia. Leukemia 2004 May; 18(5): 895-908
Vialle-Castellano A, Gaugler B, Mohty M, Isnardon D, van Baren N and D Olive. Abundant expression of fibronectin is a major feature of leukemic dendritic cells differentiated from patients with acute myeloid leukemia. Leukemia. 2004 March; 18(3): 426-433
Vialle-Castellano A, Laduron S, De Plaen E, Jost E, Dupont S, Ameye G, Michaux L, Coulie P, Olive D, Boon T and N van Baren. A gene expressed exclusively in acute B lymphoblastic leukemias. Genomics 2004 January; 83(1): 85-94
von Bergh A R M, Wijers P M, Groot A J, van Zelderen-Bhola S, Falkenburg J H F, Kluin P M and E Schuuring. Identification of a novel RAS GTPase-activating protein (RASGAP) gene at 9q34 as an MLL fusion partner in a patient with de novo acute myeloid leukemia. Genes Chromosomes Cancer 2004 April; 39(4): 324-34
Yeoh E J, Ross M E, Shurtleff S A, Williams W K, Patel D, Mahfouz R, Behm F G, Raimondi S C, Relling M V, Patel A, Cheng C, Campana D, Wilkins D, Zhou X, Li J, Liu H, Pui C H, Evans W E, Naeve C, Wong L and J R Downing. Classification, subtype discovery, and prediction of outcome in pediatric acute lymphoblastic leukemia by gene expression profiling. Cancer Cell 2002 March; 1(2): 133-143
Zhu N, Gu L, Findley H W, Li F and M Zhou. An alternatively spliced survivin variant is positively regulated by p53 and sensitizes leukemia cells to chemotherapy. Oncogene 2004 Sep. 30; 23(45): 7545-7551

Points were awarded to genes of interest based on the number of independent researchers who found modulations within lymphoma and/or leukemia, as well as the number of different methods used. For example, four scientists found CD22 to be differentially expressed between subtypes using three different techniques (microarray analysis, FISH and PCR); therefore CD22 was assigned 4 points for independent research and 3 points for different methods for a total of 7 points. Another gene, CD3D, was shown to be differentially expressed by three researchers; however they all used microarray analysis as their method. Consequently, CD3D was assigned 3 points for independent research but only one for different methods, resulting in a total score of 4 points. The identified genes were ranked and the 1154 with the highest point values were selected for inclusion in one or more HCP sets. A list of these genes is shown in Table 1.

### Example 15

### Identification of Polynucleotide Probes for Detecting Expression Levels of Genes in HCP Sets

Nucleotide sequences suitable for the preparation of polynucleotide probes for detecting expression levels of genes in HCP sets were determined as described in Example 7. The nucleotide sequences of the polynucleotide probes selected in this manner are shown in Table 20.

### Example 16

### Preparation of an Hematological Cancer Profiling (HCP) Array

The HCP array was manufactured using CodeLink technology, by GE Healthcare (USA). Briefly, the probes are manufactured and then tethered onto the array glass slide containing CodeLink's proprietary 3-D aqueous gel. This tethering step was performed in a class 1000 clean room to avoid contamination of the array slide surface. Manufactured array slides were then subjected to quality control before usage.

The HCP array slide contained 1192 probes targeting human mRNA sequences and 62 control (30 positive control and 32 negative control) probes which target bacterial sequences that are not found in humans. These control target genes are shown in Tables 29 and 30.

### Example 17

### Preparation of Test Samples from Lymphoma and Leukemia Tissues for Profiling Using an HCP Array

Test samples from 23 human lymphoma tissues, 2 leukemia tissues, and 2 control tissues were prepared. Lymphoma samples were taken from lymph nodes, leukemia samples were prepared from blood samples, and control tissues consisted of human white blood cells. The 23 lymphoma samples consisted of 5 DLBCL, 4 FL, 4 HL, 4 MCL, 2 MZL, 2 SLL and 2 peripheral TCL samples as shown in Table 42. Both the leukemia RNA samples were from CLL patients, and the 2 cell lines used were Jurkat (T-ALL) and K-562 (AML), as shown in Table 43.

TABLE 42
Lymphoma samples

| Lymphoma | Location or Cell type |
|---|---|
| DLBCL-1 | Stomach |
| DLBCL-2 | Lymph node |
| DLBCL-3 | Spleen |
| DLBCL-4 | Spleen |
| DLBCL-5 | Groin |
| FL-1 | Lymph node |
| FL-3 | Small intestine |
| FL-4 | Lymph node |
| FL-5 | Lymph node |
| HL-1 | Lymph node |
| HL-2 | Lymph node |
| HL-3 | Lymph node |
| HL-4 | Lymph node |
| MCL-1 | Lymph node |
| MCL-2 | Ileum |
| MCL-3 | Lacrimal gland |
| MCL-4 | Spleen |
| MZL-2 | Lymph node |
| MZL-4 | Lymph node |
| SLL-1 | Lymph node |
| SLL-3 | Lymph node |
| TCL-1 | Lymph node |
| TCL-2 | Testis |

TABLE 43
Leukemia samples

| | Patient sample or |
|---|---|
| Leukemia | Cell line |
| CLL-1 | Patient sample |
| CLL-2 | Patient sample |
| AML | K-562 (Cell line) |
| T-ALL | Jurkat (Cell line) |

Total RNA samples from leukemia, and control tissues were prepared using the Paxgene Blood RNA kit (Qiagen), according to the protocol supplied with the kit. Briefly, total RNA was isolated form 2.5 mL of blood. First, blood RNA tubes were thawed and incubated at room temperature for 2 hours. Next, the tubes were spun for 10 minutes and the supernatant removed. 5 mL of RNase-free water was added, followed by vortexing to resuspend pellet. The RNA tube was again spun for 10 minutes and supernatant removed. Buffers BR1 (360 μL), BR2 (300 μL) and 40 μL Proteinase K were added to the sample, vortexed and incubated for 10 min in 55° C. shaker-incubator. After incubation, the sample was spun for 3 min and supernatant was transferred to a new 1.5 mL tube. 350 μL ethanol was added, and the solution was loaded onto PAXgene column. Column was centrifuged for 1 minute and flow-through discarded. Buffers (700 μL BR3 and 2×500 μL BR4, respectively) were added to the column, spun and flow-through discarded each time. Column was transferred to new 1.5 mL tube, 40 μL Buffer BR5 added and column was spun for 1 minute. Another 40 μL Buffer BR5 was added to column and spun for 1 min. Eluate was incubated for 5 min at 65° C., chilled on ice and stored at −70° C.

Lymphoma samples were prepared from lymph node samples using the Qiagen RNeasy kit according to the protocol supplied with the kit.

Poly-A mRNA was isolated from the cell lines using the Ambion Poly(A)Purist Kit for mRNA isolation, according to the protocol supplied with the kit. Sample RNA quality was assessed using an Agilent 2100 Bioanalyzer.

Sample RNA quality from all samples was assessed using an Agilent 2100 Bioanalyzer.

Biotin-labeled cRNA was prepared from the RNA samples as follows. cRNA synthesis was performed essentially as described by Shippy et al. BMC Genomics. 2004 Sep. 2; 5(1):61. Briefly, cRNA was prepared by in vitro transcription using a single, labeled nucleotide, biotin-11-UTP in the IVT reaction at a concentration of 1.25 mM. Unlabeled UTP was present at 3.75 mM, while GTP, ATP, and CTP were at 5 mM. The mixture was incubated at 37° C. overnight for 14 hours. The labeled cRNA was then purified using an RNeasy® mini kit (Qiagen) according to the manufacturer's protocol. The concentration and quality of the cRNA were confirmed using an Agilent 2100 Bioanalyzer.

### Example 18

### Hybridization of Test Samples to HCP Array and Detection of Hybridization

Each sample was run in duplicate (and in some cases triplicate) on the HCP array. The replicates were compared to ensure the consistency of the array. Sample replicates were hybridized with the HCP array described in Example 16, using a method similar to that described by Davidson et al. Cancer Res. 2004 Sep. 15; 64(18):6797-6804. Briefly, the purified cRNA was fragmented in 5× fragmentation buffer at 94° C. for 20 minutes. 6 μg of fragmented cRNA in 60 μl of hybridization solution was added to each array chamber and incubated for 18 hours at 37° C., while shaking at 225 r.p.m. After hybridization, the arrays were washed in 0.75×TNT (0.1M Tris-Hcl, pH 7.6, 0.15M NaCl, 0.05% Tween 20) buffer at 46° C. for 1 hour followed by incubation with Cy5™-streptavidin at room temperature for 30 minutes in the dark. Arrays were then washed in 1×TNT four times for 5 minutes each followed by a rinse in 0.05% Tween 20/SSC buffer. The arrays were then dried by centrifugation, chamber units were removed and slides were kept in the dark until scanning.

Slides were scanned using the GenePix 4000B (Axon Instruments) and image analysis was carried out with the CodeLink Expression v4 software (Amersham Biosciences).

### Example 19

### Analysis of Data Generated from Hybridization of Lymphoma Test Samples to HCP Array

The gene expression profiles resulting from the hybridization of lymphoma test sample cRNA with the HCP array were analyzed using microarray analysis software to identify differentially expressed genes and establish distinct genetic profiles for lymphoma subtypes. Clustering algorithms were also employed.

Lymphoma and leukemia experiments were performed at different times and, therefore, were analyzed separately. The data analysis methods varied for the two data sets due to the differing sample sizes and subtype representation of the lymphoma and leukemia studies. The analysis of data generated from leukemia samples is described in Example 20.

Before performing analyses, expression values were normalized using the quantile normalization method so that the gene expression between samples could be compared. Technical replicates were merged by taking the average of expression values.

### Lymphoma Data Analysis

To determine the unique gene signatures for each subtype, two methods were used to generate two independent lists. Both methods used the entire discovery gene list as shown in Table 1 as a starting point and imposed a two-fold differential expression limit; however, one list was compiled manually by evaluating differences in fold change relative to the control, while the other list was generated using statistical analyses to determine genes that were significantly differentially expressed between the subtypes. For each subtype, the two lists were compared and overlapped to produce a subtype-specific gene signature.

Genes that were common to all subtypes of lymphoma, either always overexpressed or always underexpressed vs. control, were removed from subtype signatures. These genes are listed in Table 31, and may be of interest when comparing subtypes to each other, however they do not aid in distinguishing subtypes in relation to the control.

The size of the gene signatures ranged from 33 genes for HL, to 159 genes for MZL (See Tables 32-38). Because they were based on fewer samples, the signatures for MZL, SLL and TCL are larger, yet less reliable than the other subtypes. The total number of genes that make up the seven lymphoma subtype-specific signatures was determined to be 296. These genes are shown in Table 2:

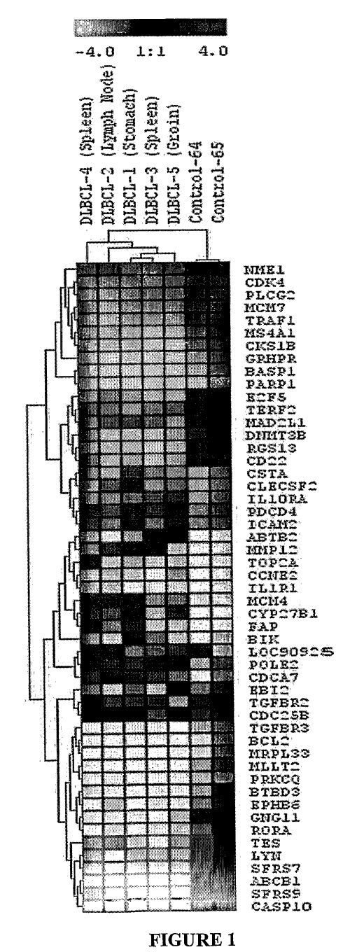

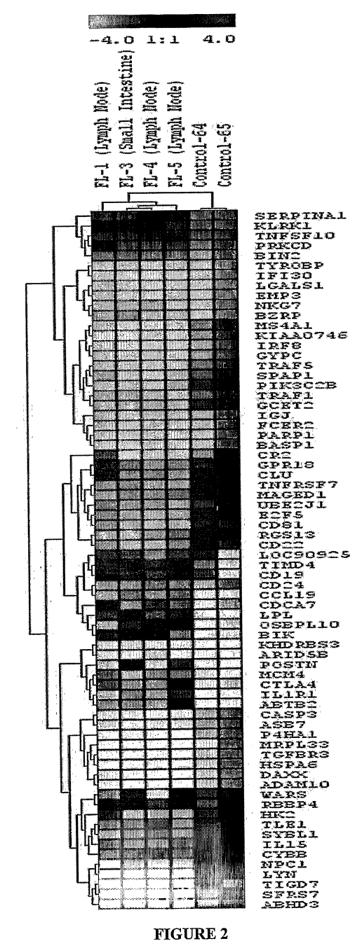

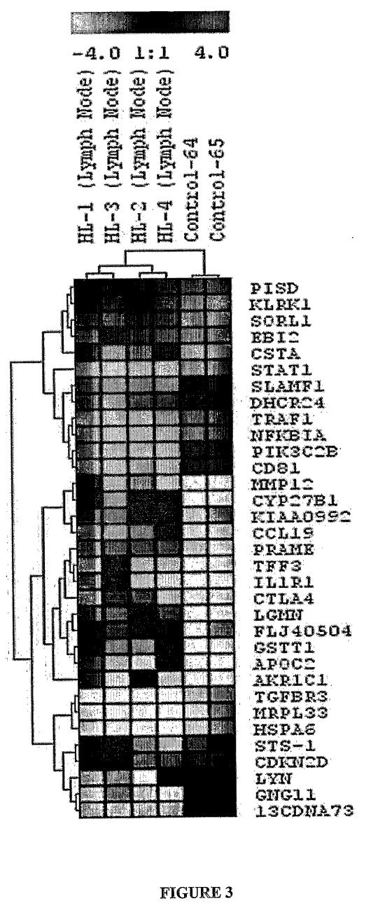

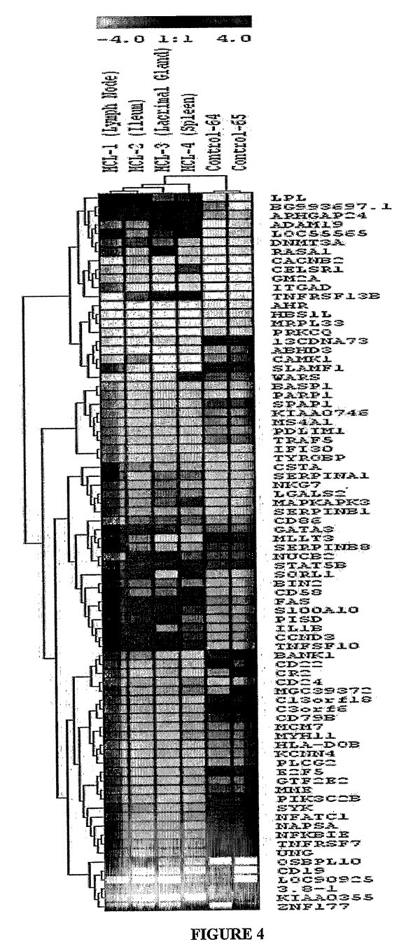

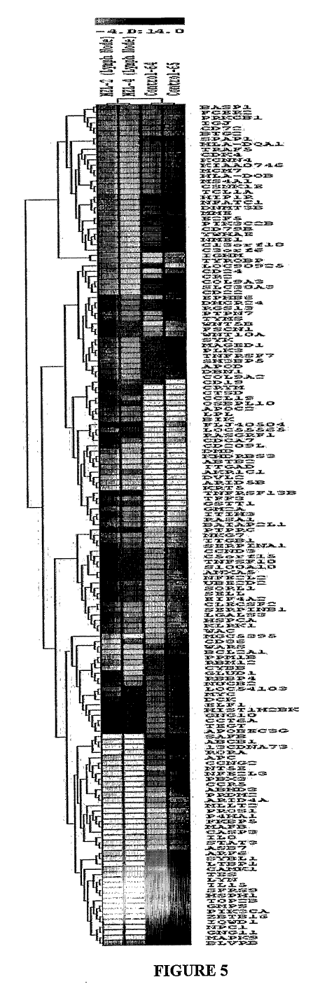

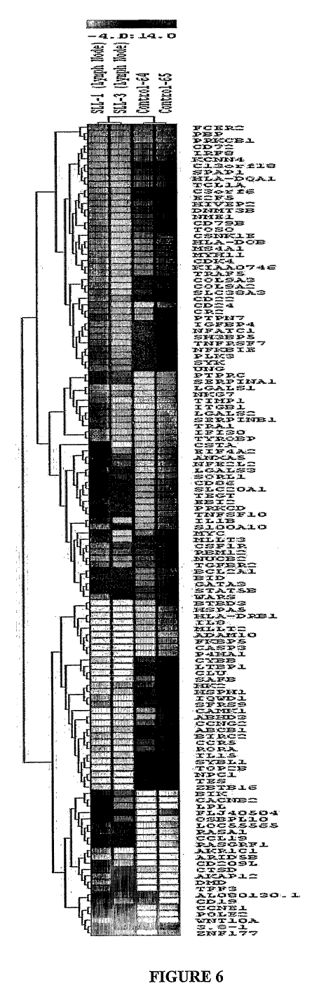

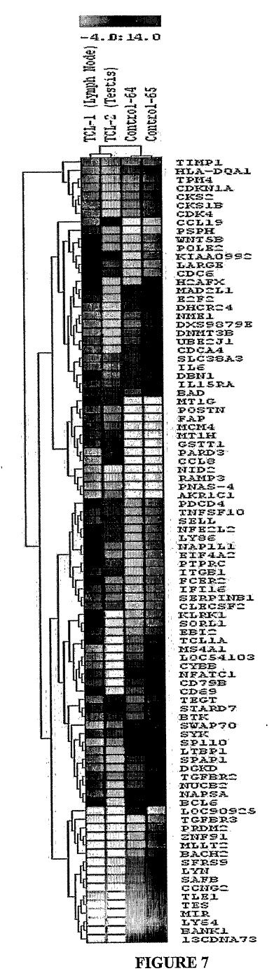

Signature genes for each lymphoma subtype are listed in Tables 32-38 and shown as hierarchical clustering images in FIGS. 1-7.

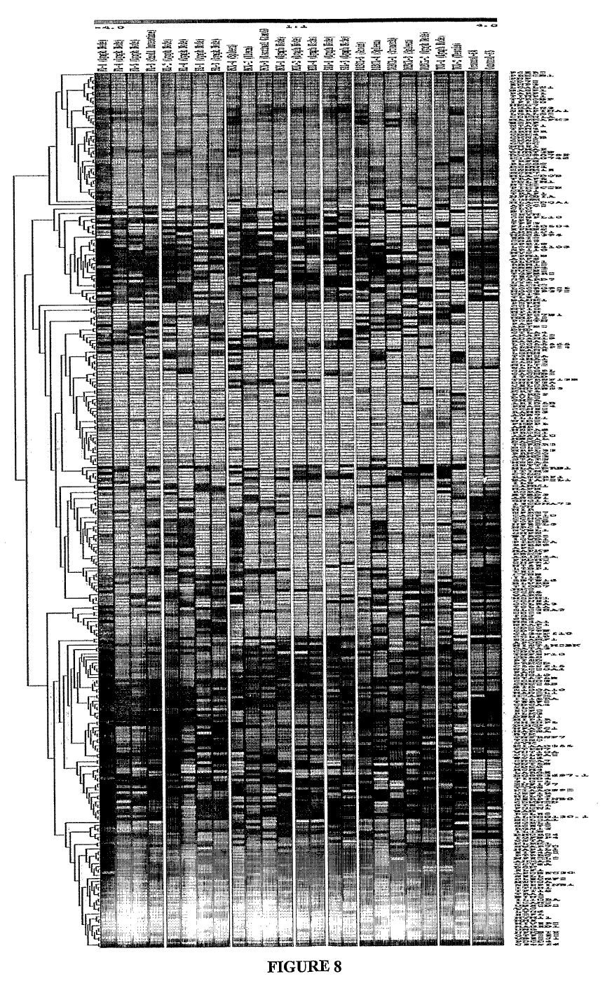

The 296 genes found to be differentially expressed between subtypes, relative to the control, were clustered using hierarchical clustering with average linkage using the Euclidian distance metrics, shown in FIG. 8. Differential gene expression is seen between lymphoma and controls as well as between the various lymphoma subtypes.

### Results

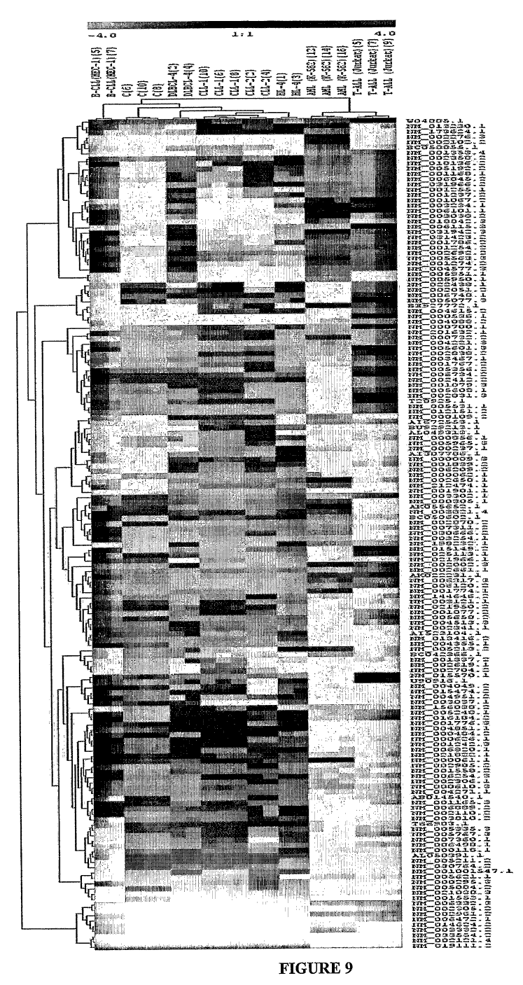

The HCP array was able to detect genes in the lymphoma and control samples. Correlation coefficient and other statistical analyses revealed that sample replicates were highly similar to one another, demonstrating the consistency of the HCP array. Statistical analyses also revealed a high dissimilarity between the lymphoma and leukemia subtypes (see Example 20) and controls, indicating that the HCP array is able to detect differential expression profiles (see FIGS. 8 and 9). As well as distinguishing between “diseased” and “healthy” samples, the HCP array identified distinct gene expression profiles that were produced by the different lymphoma and subtypes. These profiles, seen in FIGS. 1-8, provide information useful for accurate diagnosis and risk assessment.

The HCP array identified unique gene expression profiles, referred to as signatures, for each of the lymphoma subtypes tested. The following genetic signatures distinguished specific subtypes, and are listed in relation to the expression level in control samples.

### Diffuse Large B-Cell Lymphoma

Diffuse Large B-Cell Lymphoma (DLBCL) is the most common subtype of non-Hodgkin's lymphoma, accounting for 40% of annual NHL cases. DLBCL is an aggressive malignancy, with less than half of patients achieving remission. DLCBL signature genes are shown in Table 32

### Follicular Lymphoma

Follicular lymphoma (FL) is an indolent or slow growing cancer that accounts for about 25% of all non-Hodgkin's lymphomas. Due to the slow onset of FL, many patients exhibit wide-spread disease by the time they are diagnosed. Another complication of FL is that it undergoes a transformation into a more aggressive form of DLBCL in 25-60% of patients. FL signature genes are shown in Table 33.

### Hodgkin's Lymphoma

Hodgkin's Lymphoma (HL) has a fairly good prognosis, with a 20 percent mortality rate. HL is characterized by the presence of a particular type of cell morphology, known as Reed-Sternberg Cells. While HL is easier to distinguish than many subtypes, its similarity to T-Cell lymphomas, such as ALCL, still proves an obstacle to diagnosis. HL signature genes are shown in Table 34.

### Mantle Cell Lymphoma

Mantle Cell Lymphoma (MCL) accounts for only 6% of NHL's but claims for a disproportionately larger number of lives because it is an aggressive, incurable disease. Due to delayed diagnosis, many patients already have bone marrow involvement by the time MCL is identified. Some patients succumb to MCL in under a year while others survive for longer; the median survival for MCL is 3 years. MCL signature genes are shown in Table 35.

### Marginal Zone B-Cell Lymphoma

Marginal Zone B-Cell Lymphoma (MZL) is similar to MCL in appearance but progresses much more slowly. MZL can be difficult to detect, because, unlike many other lymphomas, it is not uncommon for marginal zone tumors to occur “extranodally” in areas outside the lymph nodes, such as the stomach, thyroid or bladder. MZL signature genes are shown in Table 36.

### Small Lymphocytic Lymphoma

Small Lymphocytic Lymphoma (SLL) is closely related to B-Cell Chronic Lymphocytic Leukemia (B-CLL) and tends to follow a less severe disease course than many lymphomas. However, in approximately 15% of cases, SLL transforms into aggressive DLCBL. Recent research has shown the potential of gene expression profiling to help predict these deadly transformations. SLL signature genes are shown in Table 37.

### T-Cell Lymphoma

T-Cell Lymphomas (TCL) account for only 15% of NHL, but tend to have a poor prognosis. TCL often strikes immuno-compromised individuals and is resistant to therapy. TCL signature genes are shown in Table 38.

The results of this study indicated that the above gene signatures can be used to distinguish between lymphoma subtypes thereby facilitating accurate diagnosis and timely treatment for patients.

The generated lymphoma subtype gene signatures were tested by using each signature as a reference and comparing it to the gene expression for all 23 lymphoma samples. For each sample, points were assigned for every gene that matched the reference signature, then totaled and expressed as a percentage of the total number of genes for that signature. The results of this test confirmed that the signatures were effective in classifying lymphoma subtypes. For example, the four FL samples matched the expression of the 70 genes in the FL signature for an average of 97.5%. The next closest sample, DLBCL-5, registered an 88.5% match, with most other samples showing much lower correlations. The results of this testing procedure are shown in Table 44 below.

An example of partial raw test data for HL signature genes in shown below in Table 45. Some columns were removed in order to fit onto page; this table is meant to explain how percentage scores were reached, rather than show complete data. Score columns were added for each sample, and the total score was divided by the number of genes in the signature (33 in the case of HL) to arrive at a percentage match value.

Summary of test results for subtype signatures (percentage match values for all signatures and samples)

| | | Samples | | | | | | | | | | | | |
|---|---|---|---|---|---|---|---|---|---|---|---|---|---|---|
| | | DLBCL-1 | DLBCL-2 | DLBCL-3 | DLBCL-4 | DLBCL-5 | FL-1 | FL-3 | FL-4 | FL-5 | HL-1 | HL-2 | HL-3 | HL-4 |
| Signatures | DLBCL | 98.04 | 90.20 | 98.04 | 92.16 | 94.12 | 72.55 | 54.90 | 50.98 | 52.94 | 50.98 | 50.98 | 50.98 | 33.33 |
| | FL | 68.57 | 54.29 | 61.43 | 41.43 | 88.57 | 94.29 | 98.57 | 98.57 | 98.57 | 41.43 | 62.86 | 42.86 | 55.71 |
| | HL | 42.42 | 69.70 | 72.73 | 75.76 | 78.79 | 69.70 | 72.73 | 69.70 | 66.67 | 100.00 | 100.00 | 100.00 | 90.91 |
| | MCL | 51.25 | 36.25 | 53.75 | 33.75 | 60.00 | 56.25 | 58.75 | 66.25 | 50.00 | 25.00 | 38.75 | 22.50 | 55.00 |
| | MZL | 71.07 | 63.52 | 69.81 | 32.70 | 64.15 | 77.36 | 46.54 | 62.26 | 49.69 | 51.57 | 41.51 | 46.54 | 37.74 |
| | SLL | 64.06 | 53.91 | 59.38 | 31.25 | 66.41 | 68.75 | 49.22 | 65.63 | 50.00 | 40.63 | 39.06 | 38.28 | 39.06 |
| | TCL | 60.87 | 71.74 | 70.65 | 44.57 | 52.17 | 58.70 | 27.17 | 35.87 | 38.04 | 68.48 | 38.04 | 69.57 | 16.30 |

Samples

| | | MCL-1 | MCL-2 | MCL-3 | MCL-4 | MZL-2 | MZL-4 | SLL-1 | SLL-3 | TCL-1 | TCL-2 |
|---|---|---|---|---|---|---|---|---|---|---|---|
| Signatures | DLBCL | 58.82 | 62.75 | 54.90 | 31.37 | 58.82 | 58.82 | 50.98 | 52.94 | 60.78 | 54.90 |
| | FL | 67.14 | 71.43 | 68.57 | 47.14 | 75.71 | 78.57 | 61.43 | 71.43 | 45.71 | 38.57 |
| | HL | 54.55 | 63.64 | 48.48 | 51.52 | 57.58 | 66.67 | 54.55 | 45.45 | 72.73 | 57.58 |
| | MCL | 91.25 | 97.50 | 97.50 | 92.50 | 76.25 | 78.75 | 81.25 | 78.75 | 21.25 | 37.50 |
| | MZL | 84.28 | 74.84 | 63.52 | 40.88 | 100.00 | 100.00 | 78.62 | 73.58 | 57.23 | 39.62 |
| | SLL | 84.38 | 76.56 | 74.22 | 48.44 | 82.03 | 86.72 | 100.00 | 98.44 | 47.66 | 43.75 |
| | TCL | 53.26 | 50.00 | 39.13 | 17.39 | 51.09 | 51.09 | 42.39 | 47.83 | 100.00 | 98.91 |

TABLE 45
Raw test data for HL signature genes

| Reference | | | | | | | | | | | | | | | | | |
|---|---|---|---|---|---|---|---|---|---|---|---|---|---|---|---|---|---|
| HL | | DLBCL-1 | | DLBCL-2 | | FL-4 | | FL-5 | | HL-1 | | HL-2 | | HL-3 | | HL-4 | |
| Genes | vsZ | vsZ | Score | vsZ | Score | vsZ | Score | vsZ | Score | vsZ | Score | vsZ | Score | vsZ | Score | vsZ | Score |
| CCL19 | up | up | 1 | up | 1 | up | 1 | up | 1 | up | 1 | up | 1 | up | 1 | up | 1 |
| CYP27B1 | up | up | 1 | up | 1 | null | 0 | up | 1 | up | 1 | up | 1 | up | 1 | up | 1 |
| PISD | down | null | 0 | null | 0 | null | 0 | null | 0 | down | 1 | down | 1 | down | 1 | null | 0 |
| DHCR24 | up | up | 1 | up | 1 | up | 1 | null | 0 | up | 1 | up | 1 | up | 1 | up | 1 |
| LGMN | up | null | 0 | null | 0 | up | 1 | up | 1 | up | 1 | up | 1 | up | 1 | up | 1 |
| TGFBR3 | down | down | 1 | down | 1 | down | 1 | down | 1 | down | 1 | down | 1 | down | 1 | down | 1 |
| EBI2 | down | down | 1 | down | 1 | null | 0 | null | 0 | down | 1 | down | 1 | down | 1 | null | 0 |
| TFF3 | up | null | 0 | up | 1 | up | 1 | null | 0 | up | 1 | up | 1 | up | 1 | up | 1 |
| MMP12 | up | up | 1 | up | 1 | null | 0 | null | 0 | up | 1 | up | 1 | up | 1 | up | 1 |
| LYN | down | down | 1 | down | 1 | down | 1 | down | 1 | down | 1 | down | 1 | down | 1 | null | 0 |
| IL1R1 | up | null | 0 | up | 1 | up | 1 | up | 1 | up | 1 | up | 1 | up | 1 | up | 1 |
| CD81 | up | null | 0 | null | 0 | up | 1 | up | 1 | up | 1 | up | 1 | up | 1 | up | 1 |
| APOC2 | up | null | 0 | up | 1 | null | 0 | up | 1 | up | 1 | up | 1 | up | 1 | up | 1 |
| TRAF1 | up | up | 1 | up | 1 | up | 1 | up | 1 | up | 1 | up | 1 | up | 1 | up | 1 |
| SLAMF1 | up | down | 0 | up | 1 | up | 1 | up | 1 | up | 1 | up | 1 | up | 1 | up | 1 |
| CDKN2D | down | null | 0 | down | 1 | down | 1 | down | 1 | down | 1 | down | 1 | down | 1 | down | 1 |
| PRAME | up | up | 1 | null | 0 | null | 0 | null | 0 | up | 1 | up | 1 | up | 1 | up | 1 |
| SORL1 | down | null | 0 | down | 1 | null | 0 | null | 0 | down | 1 | down | 1 | down | 1 | down | 1 |
| CSTA | down | down | 1 | null | 0 | down | 1 | null | 0 | down | 1 | down | 1 | down | 1 | down | 1 |
| HSPA6 | down | null | 0 | down | 1 | down | 1 | down | 1 | down | 1 | down | 1 | down | 1 | down | 1 |
| GSTT1 | up | null | 0 | up | 1 | up | 1 | null | 0 | up | 1 | up | 1 | up | 1 | up | 1 |
| PIK3C2B | up | up | 1 | null | 0 | up | 1 | up | 1 | up | 1 | up | 1 | up | 1 | up | 1 |
| KIAA0992 | up | null | 0 | null | 0 | null | 0 | null | 0 | up | 1 | up | 1 | up | 1 | up | 1 |
| GNG11 | down | down | 1 | down | 1 | null | 0 | down | 1 | down | 1 | down | 1 | down | 1 | down | 1 |
| 13CDNA73 | down | down | 1 | down | 1 | down | 1 | down | 1 | down | 1 | down | 1 | down | 1 | down | 1 |
| CTLA4 | up | null | 0 | up | 1 | up | 1 | up | 1 | up | 1 | up | 1 | up | 1 | up | 1 |
| MRPL33 | down | down | 1 | down | 1 | down | 1 | down | 1 | down | 1 | down | 1 | down | 1 | down | 1 |
| STAT1 | up | down | 0 | null | 0 | null | 0 | up | 1 | up | 1 | up | 1 | up | 1 | up | 1 |
| FLJ40504 | up | down | 0 | up | 1 | up | 1 | null | 0 | up | 1 | up | 1 | up | 1 | up | 1 |
| AKR1C1 | up | null | 0 | null | 0 | up | 1 | up | 1 | up | 1 | up | 1 | up | 1 | up | 1 |
| KLRK1 | down | null | 0 | down | 1 | down | 1 | down | 1 | down | 1 | down | 1 | down | 1 | down | 1 |
| NFKBIA | up | null | 0 | null | 0 | up | 1 | up | 1 | up | 1 | up | 1 | up | 1 | up | 1 |
| STS-1 | down | null | 0 | down | 1 | down | 1 | down | 1 | down | 1 | down | 1 | down | 1 | down | 1 |
| | | | 42.4 | | 69.7 | | 69.7 | | 66.7 | | 100 | | 100 | | 100 | | 90.9 |

Numerous genes on the HCP array found to be differentially expressed in certain subtypes are consistent with recent publications. For example, Rosenwald et al. showed that both BANK1 and SPAP1 were overexpressed in MCL, in agreement with the hematological cancer profiling signatures as shown in Table 35. As well, several of the DLBCL signature genes identified using the HCP array, namely the overexpression of NME1, MCM7 and BIK, have been confirmed by multiple sources.

### Example 20

### Analysis of Data Generated from Hybridization of Leukemia Test Samples to HCP Array

The gene expression profiles resulting from the hybridization of test sample cRNA with the HCP array were analyzed using microarray analysis software to identify differentially expressed genes and establish distinct genetic profiles for lymphoma subtypes. Clustering algorithms were also employed.

Lymphoma and leukemia experiments were performed at different times and, therefore, were analyzed separately. The data analysis methods varied for the two data sets due to the differing sample sizes and subtype representation of the lymphoma and leukemia studies.

Before performing analyses, expression values were normalized using the quantile normalization method so that the gene expression between samples could be compared. Technical replicates were merged by taking the average of expression values.

### Leukemia Data Analysis

SAM analysis was performed to identify genes with significant differential expression. From the resulting set of genes, a one-way ANOVA, with Stepdown Westfall-Young adjustment to p-values, was used to select genes with p<0.01. These analyses yielded a list of 157 genes that were differentially expressed between leukemia subtypes.

The 157 genes found to be differentially expressed were clustered using hierarchical clustering with average linkage using the Euclidian distance metrics, shown in FIG. 9. A list of these genes is found in Table 3 above. Differential gene expression is seen between leukemia and controls as well as between the various leukemia subtypes.

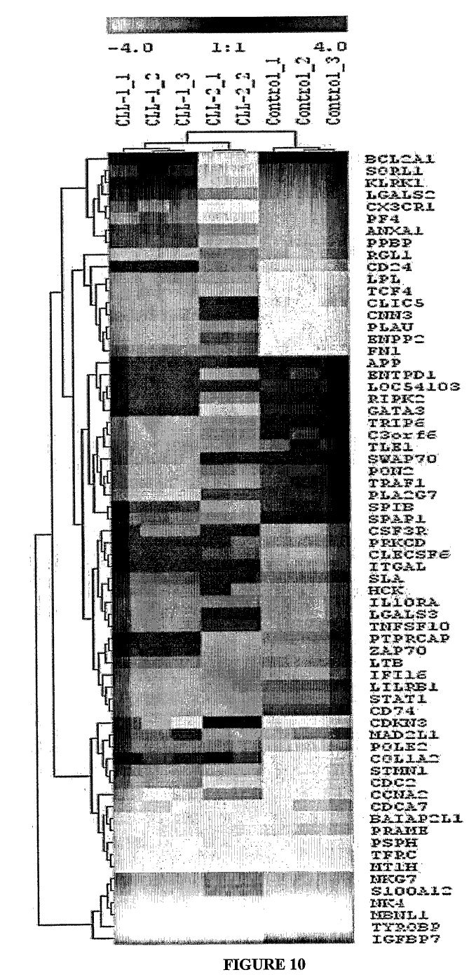

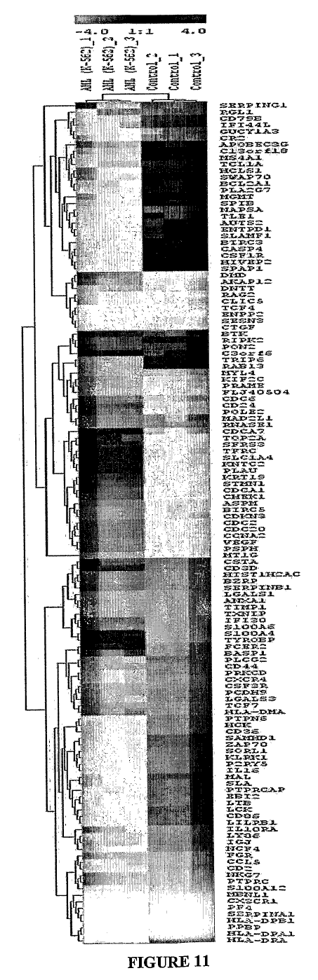

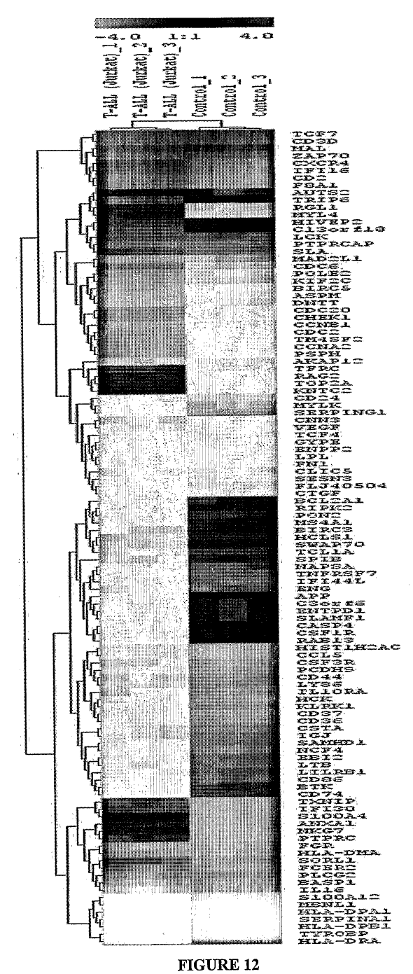

To determine the unique gene signatures for each leukemia subtype, a 5 fold filter was imposed on the 157 significant genes. Only genes that showed a fold change of greater than 5 or less than −5 in a subtype were considered to be “signature” genes. Gene lists for CLL, AML and T-ALL were each filtered to meet these criteria, thereby producing subtype-specific gene signatures. Signature genes for individual leukemia subtypes are listed in Tables 39-41 and shown as hierarchical clustering images in FIGS. 10-12.

### Results

The HCP array was able to detect genes in the leukemia and control samples. Correlation coefficient and other statistical analyses revealed that sample replicates were highly similar to one another, demonstrating the consistency of the HCP array. Statistical analyses also revealed a high dissimilarity between the lymphoma and leukemia subtypes and controls, indicating that the HCP array is able to detect differential expression profiles (see FIG. 9). As well as distinguishing between “diseased” and “healthy” samples, the HCP array identified distinct gene expression profiles that were produced by the different leukemia subtypes. These profiles, seen in FIGS. 9-12, provide information useful for accurate diagnosis and risk assessment.

The HCP array identified unique gene expression profiles, referred to as signatures, for each of the leukemia subtypes tested. The following genetic signatures distinguished specific leukemia subtypes, and are listed in relation to the expression level in control samples.

### Chronic Lymphocytic Leukemia

Chronic Lymphocytic Leukemia (CLL) is the most common adult leukemia. CLL is a cancer of the B- or T-lymphocytes; B-CLL is the prevalent form of CLL, T-lymphocyte abnormalities, while more severe, account for less than 5% of CLL cases. CLL can be difficult to diagnose, due to its slow onset and vague symptoms. The Flu-like nature of some of the common CLL symptoms, such as fever and fatigue, are often a cause of delayed or misdiagnosis.

CLL has become a topic of particular interest to physicians due to its inconsistent response to treatment. Many CLL patients respond well to chemotherapy and/or radiation, while others show virtually no improvement. Recent research by Mackey et al. Blood 2005 Jan. 15; 105(2): 767-774, and Vallat et al. Blood 2003 Jun. 1; 101(11): 4598-4606, has suggested the existence of distinct CLL subtypes that are resistant to chemotherapy and radiation treatment, respectively. Identification of new CLL subtypes is the next step in understanding and improving the prognosis of CLL. CLL signature genes are shown in Table 39.

### Acute Myelogenous Leukemia

Acute Myelogenous Leukemia (AML) patients worsen quickly, making fast and accurate diagnosis a must. Unfortunately, AML can easily be misdiagnosed using common (aforementioned) methods. Most AML patients respond well to initial treatment, however, AML has a high rate of relapse. Improved understanding of gene expression within AML will lead to efficient diagnostic tools as well as outcome prediction. AML signature genes are shown in Table 40.

### Acute T-Cell Lymphocytic Leukemia

Acute T-Cell Lymphocytic Leukemia (T-ALL) is the most common leukemia among children and adolescents. Less understood than its B-Cell counterpart, T-ALL proves difficult to classify. T-ALL does not display distinct subtypes, making risk assessment challenging. However, a recent study by Chiaretti et al. Blood 2004 Apr. 1; 103(7): 2771-2778, showed that gene expression profiles can provide insight into prognosis of T-ALL, with certain genes indicating a favorable outcome and others a high risk of relapse. T-ALL signature genes are shown in Table 41.

The results of this study indicate that the above gene signatures can be used to distinguish between leukemia subtypes thereby facilitating accurate diagnosis and timely treatment for patients.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

Although the invention has been described with reference to certain specific embodiments, various modifications thereof will be apparent to those skilled in the art without departing from the spirit and scope of the invention as outlined in the claims appended hereto.

1. A system for profiling a hematological cancer comprising at least ten polynucleotide probes, each of said probes being between about 15 and about 500 nucleotides in length and comprising a sequence corresponding to, or complementary to, an mRNA transcribed from a gene selected from the group of genes set forth in Table 1, wherein the level of expression of said gene is indicative of one or more features of said hematological cancer.

2. The system according to claim 1, wherein said one or more features are selected from the group of: presence, absence, type, subtype, stage, progression, grade, aggressivity, outcome, survival and drug-responsiveness.

3. The system according to claim 1, wherein each of said probes comprises a sequence corresponding to, or complementary to, an mRNA transcribed from a gene selected from the group of genes set forth in Tables 2-19.

4. The system according to claim 1, wherein said at least ten polynucleotide probes are selected from:
(a) at least ten polynucleotide probes comprising a sequence corresponding to, or complementary to, an mRNA transcribed from a gene from a set of genes comprising one or more genes as set forth in Table 2;
(b) at least ten polynucleotide probes comprising a sequence corresponding to, or complementary to, an mRNA transcribed from a gene from a set of genes comprising one or more genes as set forth in Table 3;
(c) at least ten polynucleotide probes comprising a sequence corresponding to, or complementary to, an mRNA transcribed from a gene from a set of genes comprising one or more genes as set forth in Table 4;
(d) at least ten polynucleotide probes comprising a sequence corresponding to, or complementary to, an mRNA transcribed from a gene from a set of genes comprising one or more genes as set forth in Table 5;
(e) at least ten polynucleotide probes comprising a sequence corresponding to, or complementary to, an mRNA transcribed from a gene from a set of genes comprising one or more genes as set forth in Table 6;
(f) at least ten polynucleotide probes comprising a sequence corresponding to, or complementary to, an mRNA transcribed from a gene from a set of genes comprising one or more genes as set forth in Table 7;
(g) at least ten polynucleotide probes comprising a sequence corresponding to, or complementary to, an mRNA transcribed from a gene from a set of genes comprising one or more genes as set forth in Table 8;
(h) at least ten polynucleotide probes comprising a sequence corresponding to, or complementary to, an mRNA transcribed from a gene from a set of genes comprising one or more genes as set forth in Table 9;
(i) at least ten polynucleotide probes comprising a sequence corresponding to, or complementary to, an mRNA transcribed from a gene from a set of genes comprising one or more genes as set forth in Table 10;
(j) at least ten polynucleotide probes comprising a sequence corresponding to, or complementary to, an mRNA transcribed from a gene from a set of genes comprising one or more genes as set forth in Table 11;
(k) at least ten polynucleotide probes comprising a sequence corresponding to, or complementary to, an mRNA transcribed from a gene from a set of genes comprising one or more genes as set forth in Table 12;
(l) at least ten polynucleotide probes comprising a sequence corresponding to, or complementary to, an mRNA transcribed from a gene from a set of genes comprising one or more genes as set forth in Table 13;
(m) at least ten polynucleotide probes comprising a sequence corresponding to, or complementary to, an mRNA transcribed from a gene from a set of genes comprising one or more genes as set forth in Table 14;
(n) at least ten polynucleotide probes comprising a sequence corresponding to, or complementary to, an mRNA transcribed from a gene from a set of genes comprising one or more genes as set forth in Table 15;
(o) at least ten polynucleotide probes comprising a sequence corresponding to, or complementary to, an mRNA transcribed from a gene from a set of genes comprising one or more genes as set forth in Table 16;
(p) at least ten polynucleotide probes comprising a sequence corresponding to, or complementary to, an mRNA transcribed from a gene from a set of genes comprising one or more genes as set forth in Table 17;
(q) at least ten polynucleotide probes comprising a sequence corresponding to, or complementary to, an mRNA transcribed from a gene from a set of genes comprising one or more genes as set forth in Table 18, and
(r) at least ten polynucleotide probes comprising a sequence corresponding to, or complementary to, an mRNA transcribed from a gene from a set of genes comprising one or more genes as set forth in Table 19.

5. The system according to claim 1, wherein each of said probes comprises at least 15 nucleotides of a sequence as set forth in any one of SEQ ID NOs: 1-4530.

6. The system according to claim 1, wherein each of said probes comprises a sequence as set forth in any one of SEQ ID NOs: 1-4530.

7. The system according to claim 1, wherein said system comprises at least 50 polynucleotide probes.

8. The system according to claim 1, wherein said system comprises at least 100 polynucleotide probes.

9. The system according to claim 1, wherein said hematological cancer is selected from the group of lymphoma and leukemia.

10. The system according to claim 1, wherein said hematological cancer is a lymphoma selected from the group of: B-cell chronic lymphocytic leukemia/small lymphocytic lymphoma (CLL/SLL), B-cell prolymphocytic leukemia, lymphoplamacytic lymphoma, splenic marginal zone B-cell lymphoma, nodal marginal zone B-cell lymphoma, hairy cell leukemia, plasma cell myeloma/plasmacytoma, follicular lymphoma (FL), mantle cell lymphoma (MCL), Burkitt's lymphoma, diffuse large cell B-cell lymphoma (DLBCL) Hodgkin's lymphoma, lymphoblastic lymphoma, anaplastic large cell lymphoma (ALCL), cutaneous T-cell lymphoma, mycosis fungoids/Sézary's syndrome, peripheral T-cell lymphomas, angioimmunoblastic lymphoma, angiocentric lymphoma (nasal T-cell lymphoma), intestinal T-cell lymphoma, and adult T-cell lymphoma/leukemia.

11. The system according to claim 1, wherein said hematological cancer is a leukemia selected from the group of: acute myelogenous leukemia, acute lymphocytic leukemia, chronic myelogenous leukemia, and chronic lymphocytic leukemia.

12. Use of the system according to claim 1, for the preparation of a nucleic acid array.

13. A method of profiling a hematological cancer in a subject comprising:
(a) providing one or more gene sets, each gene set comprising at least five genes selected from the genes set forth in Table 1, wherein the expression level of each gene in said one or more gene sets is indicative of a feature of a hematological cancer;
(b) determining the expression level of each gene in said one or more gene sets in a test sample obtained from said subject to provide an expression pattern profile, and
(c) comparing said expression pattern profile with a reference expression pattern profile.

14. The method according to claim 13, wherein said feature is selected from the group of: presence, absence, type, subtype, stage, progression, grade, aggressivity, outcome, survival and drug-responsiveness.

15. The method according to claim 13, wherein the expression level of each gene is determined in step (b) by contacting the test sample with a plurality of polynucleotide probes, each of said probes being between about 15 and about 500 nucleotides in length and comprising a sequence corresponding to, or complementary to, a gene from said one or more gene sets.

16. The method according to claim 15, wherein each of said probes comprises at least 15 nucleotides of a sequence as set forth in any one of SEQ ID NOs: 1-4530.

17. The method according to claim 13, wherein said hematological cancer is selected from the group of lymphoma and leukemia.

18. The method according to claim 13, wherein said hematological cancer is a lymphoma selected from the group of: B-cell chronic lymphocytic leukemia/small lymphocytic lymphoma (CLL/SLL), B-cell prolymphocytic leukemia, lymphoplamacytic lymphoma, splenic marginal zone B-cell lymphoma, nodal marginal zone B-cell lymphoma, hairy cell leukemia, plasma cell myeloma/plasmacytoma, follicular lymphoma (FL), mantle cell lymphoma (MCL), Burkitt's lymphoma, diffuse large cell B-cell lymphoma (DLBCL) Hodgkin's lymphoma, lymphoblastic lymphoma, anaplastic large cell lymphoma (ALCL), cutaneous T-cell lymphoma, mycosis fungoids/Sézary's syndrome, peripheral T-cell lymphomas, angioimmunoblastic lymphoma, angiocentric lymphoma (nasal T-cell lymphoma), intestinal T-cell lymphoma, and adult T-cell lymphoma/leukemia.

19. The method according to claim 13, wherein said hematological cancer is a leukemia selected from the group of: acute myelogenous leukemia, acute lymphocytic leukemia, chronic myelogenous leukemia, and chronic lymphocytic leukemia.

20. The method according to claim 13, wherein each of said gene sets comprises at least ten genes and said one or more gene sets are selected from the group of:
(a) a gene set comprising at least ten genes selected from the genes set forth in Table 32;
(b) a gene set comprising at least ten genes selected from the genes set forth in Table 33;
(c) a gene set comprising at least ten genes selected from the genes set forth in Table 34;
(d) a gene set comprising at least ten genes selected from the genes set forth in Table 35;
(e) a gene set comprising at least ten genes selected from the genes set forth in Table 36;
(f) a gene set comprising at least ten genes selected from the genes set forth in Table 37;
(g) a gene set comprising at least ten genes selected from the genes set forth in Table 38;
(h) a gene set comprising at least ten genes selected from the genes set forth in Table 39;
(i) a gene set comprising at least ten genes selected from the genes set forth in Table 40, and
(j) a gene set comprising at least ten genes selected from the genes set forth in Table 41.

21. A nucleic acid array comprising at least ten polynucleotide probes immobilized on a solid support, each of said probes being between about 15 and about 500 nucleotides in length and comprising a sequence corresponding to, or complementary to, an mRNA transcribed from a gene selected from the group of genes set forth in Table 1, wherein the level of expression of said gene is indicative of one or more features of said hematological cancer.

22. The nucleic acid array according to claim 21, wherein said one or more features are selected from the group of: presence, absence, type, subtype, stage, progression, grade, aggressivity, outcome, survival and drug-responsiveness.

23. The nucleic acid array according to claim 21, wherein each of said probes comprises at least 15 nucleotides of a sequence as set forth in any one of SEQ ID NOs: 1-4530.

24. The nucleic acid array according to claim 21, wherein each of said probes comprises a sequence as set forth in any one of SEQ ID NOs: 1-4530.

25. The nucleic acid array according to claim 21, wherein each of said probes comprises a sequence as set forth in any one of SEQ ID NOs: 1-1153.

26. The nucleic acid array according to claim 21, wherein each of said probes comprises a sequence as set forth in any one of SEQ ID NOs: 1154-2299.

27. The nucleic acid array according to claim 21, wherein each of said probes comprises a sequence as set forth in any one of SEQ ID NOs: 2300-3426.

28. The nucleic acid array according to claim 21, wherein each of said probes comprises a sequence as set forth in any one of SEQ ID NOs: 3427-4530.

29. The nucleic acid array according to claim 21, wherein said nucleic acid array comprises at least 50 polynucleotide probes.

30. The nucleic acid array according to claim 21, wherein said nucleic acid array comprises at least 100 polynucleotide probes.

31. The nucleic acid array according to claim 21, wherein each of said probes comprises a sequence corresponding to, or complementary to, an mRNA transcribed from a gene selected from the group of genes set forth in Table 2.

32. The nucleic acid array according to claim 21, wherein each of said probes comprises a sequence corresponding to, or complementary to, an mRNA transcribed from a gene selected from the group of genes set forth in Table 3.

33. The nucleic acid array according to claim 21, wherein said at least ten polynucleotide probes are selected from:
(a) at least ten polynucleotide probes comprising a sequence corresponding to, or complementary to, an mRNA transcribed from a gene from a set of genes comprising one or more genes as set forth in Table 32;
(b) at least ten polynucleotide probes comprising a sequence corresponding to, or complementary to, an mRNA transcribed from a gene from a set of genes comprising one or more genes as set forth in Table 33;
(c) at least ten polynucleotide probes comprising a sequence corresponding to, or complementary to, an mRNA transcribed from a gene from a set of genes comprising one or more genes as set forth in Table 34;
(d) at least ten polynucleotide probes comprising a sequence corresponding to, or complementary to, an mRNA transcribed from a gene from a set of genes comprising one or more genes as set forth in Table 35;
(e) at least ten polynucleotide probes comprising a sequence corresponding to, or complementary to, an mRNA transcribed from a gene from a set of genes comprising one or more genes as set forth in Table 36;
(f) at least ten polynucleotide probes comprising a sequence corresponding to, or complementary to, an mRNA transcribed from a gene from a set of genes comprising one or more genes as set forth in Table 37;
(g) at least ten polynucleotide probes comprising a sequence corresponding to, or complementary to, an mRNA transcribed from a gene from a set of genes comprising one or more genes as set forth in Table 38;
(h) at least ten polynucleotide probes comprising a sequence corresponding to, or complementary to, an mRNA transcribed from a gene from a set of genes comprising one or more genes as set forth in Table 39;
(i) at least ten polynucleotide probes comprising a sequence corresponding to, or complementary to, an mRNA transcribed from a gene from a set of genes comprising one or more genes as set forth in Table 40, and
(j) at least ten polynucleotide probes comprising a sequence corresponding to, or complementary to, an mRNA transcribed from a gene from a set of genes comprising one or more genes as set forth in Table 41.

34. The nucleic acid array according to claim 21, further comprising one or more control probes.

35. A polynucleotide probe between about 15 and about 500 nucleotides in length and comprising a sequence corresponding to, or complementary to, an mRNA transcribed from a gene selected from the group of genes set forth in Table 1, wherein said probe comprises at least 15 consecutive nucleotides of a sequence as set forth in any one of SEQ ID NOs: 1-4530.

36. The polynucleotide probe according to claim 35, wherein said polynucleotide probe comprises a sequence as set forth in as set forth in SEQ ID NOs: 1-4530.

37. The polynucleotide probe according to claim 35, wherein said polynucleotide probe consists of a sequence as set forth in any one of SEQ ID NOs: 1-4530.

38. A set of genes having an expression pattern representative of one or more features of a hematological cancer and comprising at least ten genes selected from:
(a) at least ten genes selected from the genes set forth in Table 32;
(b) at least ten genes selected from the genes set forth in Table 33;
(c) at least ten genes selected from the genes set forth in Table 34;
(d) at least ten genes selected from the genes set forth in Table 35;
(e) at least ten genes selected from the genes set forth in Table 36;
(f) at least ten genes selected from the genes set forth in Table 37;
(g) at least ten genes selected from the genes set forth in Table 38;
(h) at least ten genes selected from the genes set forth in Table 39;
(i) at least ten genes selected from the genes set forth in Table 40, and
(j) at least ten genes selected from the genes set forth in Table 41.

39. The set of genes according to claim 38, wherein said one or more features are selected from the group of: presence, absence, type, subtype, stage, progression, grade, aggressivity, outcome, survival and drug-responsiveness.

40. The set of genes according to claim 38, wherein said hematological cancer is a lymphoma and said at least ten genes are selected from:
(a) at least ten genes selected from the genes set forth in Table 32;
(b) at least ten genes selected from the genes set forth in Table 33;
(c) at least ten genes selected from the genes set forth in Table 34;
(d) at least ten genes selected from the genes set forth in Table 35;
(e) at least ten genes selected from the genes set forth in Table 36;
(f) at least ten genes selected from the genes set forth in Table 37; and
(g) at least ten genes selected from the genes set forth in Table 38.

41. The set of genes according to claim 38, wherein said hematological cancer is a leukemia and said at least ten genes are selected from:
(a) at least ten genes selected from the genes set forth in Table 39;
(b) at least ten genes selected from the genes set forth in Table 40, and
(c) at least ten genes selected from the genes set forth in Table 41.

42. A library of genes for profiling a hematological cancer, comprising the genes as set forth in Table 1.

43. A computer-readable medium comprising one or more digitally-encoded expression pattern profiles representative of the set of genes according to claim 38, each of said one or more expression pattern profiles being associated with one or more values wherein each of said one or more values is correlated with one of said one or more features of a hematological cancer.
